(19) United States
(12) Patent Application Publication
(10) Pub. No.: US 2024/0105279 A1
(43) Pub. Date: Mar. 28, 2024

(54) METHODS AND SYSTEMS EMPLOYING TARGETED NEXT GENERATION SEQUENCING FOR CLASSIFYING A TUMOR SAMPLE AS HAVING A LEVEL OF HOMOLOGOUS RECOMBINATION DEFICIENCY SIMILAR TO THAT ASSOCIATED WITH MUTATIONS IN BRCA1 OR BRCA2 GENES

(71) Applicant: GENOMIC TESTING COOPERATIVE, LCA, Irvine, CA (US)

(72) Inventors: Maher Albitar, Valley Center, CA (US); Hong Zhang, Savannah, GA (US)

(73) Assignee: GENOMIC TESTING COOPERATIVE, LCA, Irvine, CA (US)

(21) Appl. No.: 18/368,320

(22) Filed: Sep. 14, 2023

Related U.S. Application Data
(60) Provisional application No. 63/407,030, filed on Sep. 15, 2022.

(51) Int. Cl. G16B 20/10; G16B 20/20; G16B 40/20

(52) U.S. Cl. CPC G16B 20/10; G16B 20/20; G16B 40/20

(57) ABSTRACT

Methods and systems for classifying a patient sample as having a mutation in a BRCA1 or BRCA2 gene or as having genomic structural abnormalities indicating a similar level of homologous recombination deficiency (HRD) as that caused by a mutation in a BRCA1 or BRCA2 gene based on copy number variation determined from next generation sequencing (NGS).

### CROSS-REFERENCE TO RELATED APPLICATIONS

This application claims the benefit of priority to U.S. Provisional Application No. 63/407,030 (filed Sep. 15, 2022), which is incorporated herein by reference in its entirety.

### FIELD OF THE DISCLOSURE

The present disclosure relates to systems and method for identifying a patient sample as having a mutation in a BRCA1 or BRCA2 gene or demonstrating a level of homologous recombination deficiency similar to that of a patient sample having a mutation in a BRCA1 or BRCA2 gene.

### BACKGROUND OF THE INVENTION

Homologous recombination deficiency (HRD) is the hallmark of BRCA1/2-mutated tumors and the unique biomarker for predicting response to double strand break (DSB)-inducing drugs. The demonstration of HRD in tumors with mutations in genes other than BRCA1/2 is considered the best biomarker of potential response to these DSB-inducer drugs. Unfortunately, most of the tests for the presence of HRD require extensive and complicated genomic evaluation.

As noted above, the presence of homologous recombination deficiency (HRD) due to DNA double strand break (DSB) repair deficiency is the hallmark of cancers that carry abnormalities in BRCA1/2 genes [Tarsounas M, Sung P. The antitumorigenic roles of BRCA1-BARD1 in DNA repair and replication. Nat Rev Mol Cell Biol. 2020; 21(5):284-299. doi: 10.1038/s41580-020-0218-z; Sharma R, Lewis S, Wlodarski M W. DNA Repair Syndromes and Cancer: Insights Into Genetics and Phenotype Patterns. Front Pediatr. 2020; 8:570084. doi: 10.3389/fped.2020.570084. eCollection 2020; Jensen R B, Rothenberg E.

Preserving genome integrity in human cells via DNA double-strand break repair. Mol Biol Cell. 2020 Apr. 15; 31(9):859-865. doi: 10.1091/mbc.E18-10-0668; Iliakis G. et al. Mechanisms of DNA double strand break repair and chromosome aberration formation. Cytogenet Genome Res. 2004; 104(1-4):14-20. doi: 10.1159/000077461]. The demonstration of HRD has been accepted as a biomarker for response to DSB-inducing drugs, including platinum salts and poly ADP-ribose polymerase inhibitors (PARPi) [Hodgson D R, et al. Candidate biomarkers of PARP inhibitor sensitivity in ovarian cancer beyond the BRCA genes. Br J Cancer. 2018 November; 119(11):1401-1409. doi: 10.1038/s41416-018-0274-8. Epub 2018 Oct 24. PMID: 30353044; Ray Chaudhuri A, Nussenzweig A. The multifaceted roles of PARP1 in DNA repair and chromatin remodeling. Nat Rev Mol Cell Biol. 2017 October; 18(10):610-621. doi: 10.1038/nrm.2017.53. Epub 2017 Jul 5.].

Typically, the presence of a germline mutation in BRCA1/2 is considered the gold standard biomarker for response to PARPi. [Abkevich V, et al. Patterns of genomic loss of heterozygosity predict homologous recombination repair defects in epithelial ovarian cancer. Br J Cancer. 2012 Nov. 6; 107(10):1776-82. doi: 10.1038/bjc.2012.451. Epub 2012 Oct 9. PMID: 23047548; Kadouri L, et al. Homologous recombination in lung cancer, germline and somatic mutations, clinical and phenotype characterization. Lung Cancer. 2019 November; 137:48-51. doi: 10.1016/j.lungcan.2019.09.008. Epub 2019 Sep 12. PMID: 31542568; Foote J R, et al. Targeted composite value-based endpoints in platinum-sensitive recurrent ovarian cancer. Gynecol Oncol. 2019 March; 152(3):445-451. doi: 10.1016/j.ygyno.2018.11.028. PMID: 30876487; Li Y, et al. Development of a Genomic Signatures-Based Predictor of Initial Platinum-Resistance in Advanced High-Grade Serous Ovarian Cancer Patients. Front Oncol. 2021 Mar. 5; 10:625866. doi: 10.3389/fonc.2020.625866. eCollection 2020; da Costa AABA, et al. Presented homologous recombination deficiency and copy number imbalances of CCNE1 and RB1 genes. BMC Cancer. 2019 May 6; 19(1):422. doi: 10.1186/s12885-019-5622-4; Wong W, et al. BRCA Mutations in Pancreas Cancer: Spectrum, Current Management, Challenges and Future Prospects. Cancer Manag Res. 2020 Apr. 23; 12:2731-2742. doi: 10.2147/CMAR.S211151. eCollection 2020].

Most of the testing for the presence of HRD is based on the presence of BRCA1/BRCA2 mutations or the presence of mutations in genes involved in the DSB, such as PALB2 or RAD5.

Alternatively, it has been suggested that the effects of HRD can be demonstrated by the so-called genomic scars detected in the genome of a cancer resulting from the oncogenesis driven by HRD. This approach is also believed to be specifically helpful in cases where the gene involved in the DSB repair is inactivated by a mechanism other than mutations such as methylation or deletion.

The evaluation of these genomic scars is based on assessing chromosomal structural alterations that are typically detected when BRCA1/2 genes are mutated and driving oncogenesis. This approach allows for the detection of BRCA-like tumors that may be responsive to DSB-inducing drugs.

Genomic scars characteristic for homologous recombination repair deficiency (HRD) include TAI, LOH and LSTs. Most of the studies addressing the detection of BRCA-like effects in cancers are based on evaluating loss of heterozygosity (LOH) as well as structural rearrangements. It has been documented that the level of chromosomal aberrations correlates with HRD status. In an early study [Coleman, R L, et al. Rucaparib maintenance treatment for recurrent ovarian carcinoma after response to platinum therapy (ARIEL3): a randomised, double-blind, placebo-controlled, phase 3 trial. Lancet 2017; 390 (10106), 1949-1961], patients with high LOH (>16%) showed some improved response to PARP inhibitor (rucaparib) as compared with placebo control, but not as good as that seen in the BRCA-mutated group. This suggested that LOH is associated with a higher likelihood of response to DSB-inducing agents, but is not an adequate biomarker for selecting patients for such therapy. Subsequent studies added to the level of LOH deletion of stretches larger than 15 Mb but smaller than the whole chromosome [Abkevich V, et al. Patterns of genomic loss of heterozygosity predict homologous recombination repair defects in epithelial ovarian cancer. Br J Cancer. 2012 Nov. 6; 107(10):1776-82. doi: 10.1038/bjc.2012.451. Epub 2012 Oct 9. PMID], telomeric allelic imbalance (TAI), and large-scale transitions (LST). The addition of these additional abnormalities significantly improved the prediction of the presence of BRCA1/2-associated tumors [Gonzalez-Martin, A., et al. Niraparib in Patients with Newly Diagnosed Advanced Ovarian Cancer. N. Engl. J. Med. 2019; 381: 2391-2402; Stronach, E. A., et al., Biomarker Assessment of HR Deficiency, Tumor BRCA1/2 Mutations, and CCNE] Copy Number in Ovarian Cancer: Associations with Clinical Outcome Following Platinum Monotherapy. Mol. Cancer Res. 2018; 16(7): 1103-1111].

Telomeric allelic imbalance (TAI) evaluates if the paternal and maternal alleles are equal; Large-scale state transitions (LST) evaluates chromosomal aberrations involving large chromosomal regions more than 10 Mb apart [Gonzalez-Martin, A., et al. Niraparib in Patients with Newly Diagnosed Advanced Ovarian Cancer. N. Engl. J. Med. 2019; 381: 2391-2402; Stronach, E. A., et al., Biomarker Assessment of HR Deficiency, Tumor BRCA1/2 Mutations, and CCNE] Copy Number in Ovarian Cancer: Associations with Clinical Outcome Following Platinum Monotherapy. Mol. Cancer Res. 2018; 16(7): 1103-1111]. This combination of abnormalities generates a score that is currently used for selecting patients for therapy with DSB-inducer drugs. In a retrospective study of chemotherapy in breast and ovarian cancer, patients with known BRCA1/2 status were used as control. A score of 42 showed good prediction of BRCA1/2 mutation, and HRD was a significant predictor of residual cancer burden and pathologic complete response when BRCA1/2 were included, but it was borderline statistically relevant when BRCA1/2 nonmutated cases were considered [Telli, M. L., et al. Homologous Recombination Deficiency (HRD) Score Predicts Response to Platinum-Containing Neoadjuvant Chemotherapy in Patients with Triple-Negative Breast Cancer. Clinical Cancer Research. 2016; 22(15): 3764-3773].

Multiple subsequent studies in breast and ovarian cancer have also studied such scores and demonstrated that the presence of BRCA mutation is the best predictor of response to SB-inducers; having a low HRD score in a non-BRCA1/2 tumor can be used as an indicator of poor response to PARPi.

A more recent study (SWOG S9313 phase 3 study) [Sharma, P., et al. Impact of homologous recombination deficiency biomarkers on outcomes in patients with triple-negative breast cancer treated with adjuvant doxorubicin and cyclophosphamide (SWOG S9313). Annals of Oncology 2018 Mar 1, 29(3), 654-660] has demonstrated that disease-free survival (DFS) was better for triple-negative breast cancer patients with HRD in general as compared to patients without HRD. Unfortunately, the design of this study did not allow for determining the predictive value of the HRD score by itself.

Overall, in most of the subsequent studies in both high-grade serous ovarian or endometrial cancers and triple-negative breast cancer it appeared that value of the HRD score was of limited clinical value, and current data did not justify using it in routine clinical practice. This most likely reflects poor reliability of the scoring system and possible technical problems in evaluating TAI, LST, and LOH.

Different approaches have been explored to evaluate HRD including whole-genome sequencing (WGS), comparative genomic hybridization (CGH), and expression profiling and functional assays. Most of these approaches are cumbersome and introduce numerous new variables that complicate the potential of reproducibility and reliability of such assays.

### SUMMARY OF THE INVENTION

Some embodiments herein provide methods, systems and computer-readable media for: classifying a sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved; classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene; or classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene. Some embodiments herein provide methods for treating a subject with cancer. Some embodiments herein provide methods, systems and computer-readable media for identifying a subject as as a candidate for treatment with a double strand break-inducing agent.

According to one aspect, the described invention provides a method comprising: a) accessing or obtaining a sample from a subject who has a cancer; b) determining sequences of segments and copy number for the sequences of a plurality of target genes in the sample using next generation sequencing; c) determining copy number variation for each of a plurality of target segments from the determined sequences and copy number for the sequences of the plurality of target genes; and d) at least one of: (1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or (2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or (3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene by applying the trained classifier and using the copy number variation for the plurality of target segments as input attributes for the trained classifier.

In some embodiments, the method also comprises e) at least one of: (1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or (2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; or (3) where the sample is classified as having a mutation of the BRCA1 or the BRCA2 gene or a genomic structural abnormality biologically similar to a mutation of the BRCA1 or the BRCA2 gene indicating a similar level of HRD as that caused by a mutation of the BRCA1 or the BRCA2 gene irrespective of the mutated gene involved, identifying the subject as a candidate for treatment with a double strand break-inducing agent.

In some embodiments, the method further comprises f) at least one of (1) where the sample is classified as not having a high level of HRD and therefore not having the same or similar structural abnormality as a level of HRD associated with a mutation of the BRCA1 or the BRCA2 gene irrespective of the mutated gene involved, identifying the subject as not a candidate for treatment with a double strand break-inducing agent; or (2) where the sample is classified as not having a mutation of the BRCA1 or the BRCA2 gene or a level of HRD similar to that caused by a mutation of the BRCA1 or the BRCA2 gene irrespective of the mutated gene involved, identifying the subject as not a candidate for treatment with a double strand break-inducing agent; or (3) where the sample is classified as not having a mutation of the BRCA1 or the BRCA2 gene or a genomic structural abnormality biologically similar to a mutation of the BRCA1 gene or the BRCA2 gene indicating a similar level of HRD as that 6 caused by a mutation of the BRCA1 gene or the BRCA2 gene irrespective of the mutated gene involved, identifying the subject as not a candidate for treatment with a double strand break-inducing agent.

According to another aspect, the described invention provides a method for treating a subject with a cancer. The method comprises: a) accessing or obtaining a sample from the subject; b) determining sequences of segments and copy number for the sequences for a plurality of target genes in the sample using next generation sequencing; c) determining copy number variation for each of a plurality of target segments from the determined copy number for the sequences for the plurality of target genes; and d) at least one of: (1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or (2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or (3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 gene or the BRCA2 gene by applying the trained classifier and using the copy number variation for the plurality of target segments as input attributes for the trained classifier; e) at least one of: (1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or (2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; (3) where the sample is classified as having a mutation of the BRCA1 gene or the BRCA2 gene or a genomic structural abnormality biologically similar to a mutation of the BRCA1 gene or the BRCA2 gene indicating a similar level of HRD as that caused by a mutation of the BRCA1 gene or the BRCA2 gene irrespective of the mutated gene involved, identifying the subject as a candidate for treatment with a double strand break-inducing agent; and f) administering a therapeutic amount of a double strand break-inducing agent to the subject identified as a candidate for treatment with a double strand break-inducing agent.

In some embodiments, identifying the subject as a candidate for treatment with a double strand break-inducing agent comprises one or more of: displaying on a graphical user interface an identification of the subject as a candidate for treatment with a double strand break-inducing agent; storing data identifying the subject as a candidate for treatment with a double strand break-inducing agent; sending an electronic communication including an identification of the subject as a candidate for treatment with a double strand break-inducing agent; displaying on a graphical user interface a recommendation of treatment with a double strand break-inducing agent chemotherapy or immunotherapy for the subject; storing data including a recommendation of treatment with a double strand break-inducing agent chemotherapy or immunotherapy for the subject; or sending an electronic communication including a recommendation of treatment with a double strand break-inducing agent chemotherapy or immunotherapy for the subject.

In some embodiments, the method further comprises training a classifier to produce the trained classifier.

In some embodiments, the method further comprises determining the plurality of target segments whose copy number variation is used for classification by steps including: (A) accessing or obtaining training samples including a first group of samples with confirmed mutations in the BRCA1 gene and/or the BRCA2 gene, and a second group of samples that are confirmed negative for mutations in the BRCA1 gene and the BRCA2 gene and confirmed negative for mutations in any double strand break repair genes; (B) for each training sample, (i) determining sequences and copy number of a plurality of candidate genes in the training sample using next generation sequencing; and (ii) determining copy number variation for each of a plurality of candidate segments from the determined sequences and copy number of the plurality of candidate genes; (C) dividing the copy number variation data from all training samples for all candidate segments into k subgroups, where k is a preselected number of folds; (D) for each candidate segment, determining a mean classification error for the candidate segment, the determining including: (i) for each of the k folds: (1) designating a new one of the k-subgroups as an excluded testing subgroup and designate the remaining k-1 subgroups as training subgroups; (2) training a naïve Bayesian (NB) classifier for the fold using the copy number variation data for the candidate segment in the k-1 training subgroups and testing the trained NB classifier using the copy number variation data for the one testing subgroup; and (3) determining a classification error for the fold based on the results of testing; (ii) determining the mean classification error for the candidate segment across the folds based on the classification error for each fold; (E) selecting a current most relevant subset of the candidate segments based on the mean classification error for each candidate segment with the lowest mean classification error corresponding to the most relevant candidate segment; (F) dividing the copy number variation data from all training samples for the selected most current relevant subset of the candidate segments subset of top scoring candidate segments into m subgroups, where m is a preselected number of folds, or using the same k folds and k subgroups as above for subsequent steps regarding m subgroups and m folds; (G) training a geometric mean naïve Bayesian (GMNB) classifier based on the current most relevant subset of the candidate segments, and determining a mean measure of effectiveness based on an Area under the ROC curve (AUC) for the trained GMNB classifier across the m folds for the current most relevant subset of the candidate segments, including: (i) for each of the m folds: (1) designating a new one of the m-subgroups as an excluded testing subgroup and designating the remaining m-1 subgroups as training subgroups; (2) training a GMNB classifier for the fold using the copy number variation data for the candidate segment in the m-1 training subgroups; and (3) testing the trained GMNB classifier for the fold using the copy number variation data for the excluded testing subgroup resulting in a measure of effectiveness of the trained GMNB classifier for the fold; and (ii) determining a mean measure of effectiveness of the trained GMNB classifier across the folds for the current most relevant subset of the candidate segments, which is referred to as the current measure of effectiveness for the current most relevant subset of the candidate segments; (H) removing one or more of the least relevant candidate segments from the current most relevant subset of the candidate segments changing it into an immediately prior most relevant subset of candidate segments and forming a new current most relevant subset of the candidate segments, and labeling the current measure of effectiveness as the immediately prior measure of effectiveness for the immediately prior most relevant set of candidate segments; and (I) repeating (G) for the new current most relevant subset of the candidate segments to determine a current measure of effectiveness for the current most relevant subset of the candidate segments; where the current measure of effectiveness for the current most relevant subset of the candidate segments is statistically worse than the immediately prior measure of effectiveness for the immediately prior most relevant set of candidate segments, select the immediately prior most relevant set of candidate segments as the plurality of target segments; where the current measure of effectiveness for the current most relevant subset of the candidate segments is better than or statistically the same as the immediately prior measure of effectiveness for the immediately prior most relevant set of candidate segments, performing (H) and (I) until the current measure of effectiveness for the current most relevant subset of the candidate segments is worse than the immediately prior measure of effectiveness for the immediately prior most relevant set of candidate segments.

In some embodiments, the method further comprises training a GMNB classifier on the plurality of target segments for some of the training data, for all of the training data, or for new training data to produce the trained classifier.

According to another aspect, the invention provides a non-transitory computer-readable medium storing instructions that when executed by one or more processors of a computing system, perform at least some of the steps or all of the steps of any the methods disclosed, described, or claimed herein.

According to another aspect, the invention provides a system comprising: storage; one or more processors in communication with the storage and configured to execute instructions from the storage, that, when executed, provide one or more modules including: a sequencing and copy number variation (CNV) module configured to determine sequences and copy number for the sequences for a plurality of target genes in a sample from a subject who has a cancer using next generation sequencing; and a classification module configured to: obtain CNV data for a plurality of target sequences from the sequencing and CNV module; and at least one of: (1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or (2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or (3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene by applying the trained classifier and using the copy number variation for the plurality of target segments as input attributes for the trained classifier.

In some embodiments, the classification module is further configured to perform one or more of: (1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or (2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; or (3) where the sample is classified as having a mutation of the BRCA1 or the BRCA2 gene or a genomic structural abnormality biologically similar to a mutation of the BRCA1 or the BRCA2 gene indicating a similar level of HRD as that caused by a mutation of the BRCA1 or the BRCA2 gene irrespective of the mutated gene involved, identifying the subject as a candidate for treatment with a double strand break-inducing agent.

In some embodiments, the classification module is further configured to perform at least one of: (1) where the sample is classified as not having a high level of HRD and therefore not having the same or similar structural abnormality as a level of HRD associated with a mutation of the BRCA1 or the BRCA2 gene irrespective of the mutated gene involved, identifying the subject as not a candidate for treatment with a double strand break-inducing agent; or (2) where the sample is classified as not having a mutation of the BRCA1 or the BRCA2 gene or a level of HRD similar to that caused by a mutation of the BRCA1 or the BRCA2 gene irrespective of the mutated gene involved, identifying the subject as not a candidate for treatment with a double strand break-inducing agent; or (3) where the sample is classified as not having a mutation of the BRCA1 or the BRCA2 gene or a genomic structural abnormality biologically similar to a mutation of the BRCA1 gene or the BRCA2 gene indicating a similar level of HRD as that caused by a mutation of the BRCA1 gene or the BRCA2 gene irrespective of the mutated gene involved, identifying the subject as not a candidate for treatment with a double strand break-inducing agent.

In some embodiments, the system further comprises a classifier training module configured to produce the trained classifier. In some embodiments, producing the trained classifier comprises determining the plurality of target segments whose copy number variation is used for classification by steps including: (A) accessing or obtaining training samples including a first group of samples with confirmed mutations in the BRCA1 and/or the BRCA2 gene, and a second group of samples that are confirmed negative for mutations in the BRCA1 and the BRCA2 gene and confirmed negative for mutations in any double strand break repair genes; (B) for each training sample, (i) determining sequences and copy number of a plurality of candidate genes in the training sample using next generation sequencing; and (ii) determining copy number variation for each of a plurality of candidate segments from the determined sequences and copy number of the plurality of candidate genes; (C) dividing the copy number variation data from all training samples for all candidate segments into k subgroups, where k is a preselected number of folds; (D) for each candidate segment, determining a mean classification error for the candidate segment, the determining including: (i) for each of the k folds: (1) designating a new one of the k-subgroups as an excluded testing subgroup and designate the remaining k-1 subgroups as training subgroups; (2) training a naïve Bayesian (NB) classifier for the fold using the copy number variation data for the candidate segment in the k-1 training subgroups and testing the trained NB classifier using the copy number variation data for the one testing subgroup; and (3) determining a classification error for the fold based on the results of testing; (ii) determining the mean classification error for the candidate segment across the folds based on the classification error for each fold; (E) selecting a current most relevant subset of the candidate segments based on the mean classification error for each candidate segment with the lowest mean classification error corresponding to the most relevant candidate segment; (F) dividing the copy number variation data from all training samples for the selected most current relevant subset of the candidate segments subset of top scoring candidate segments into m subgroups, where m is a preselected number of folds, or using the same k folds and k subgroups as above for subsequent steps regarding m subgroups and m folds; (G) training a geometric mean naïve Bayesian (GMNB) classifier based on the current most relevant subset of the candidate segments, and determining a mean measure of effectiveness based on an Area under the ROC curve (AUC) for the trained GMNB classifier across the m folds for the current most relevant subset of the candidate segments, including: (i) for each of the m folds: (1) designating a new one of the m-subgroups as an excluded testing subgroup and designating the remaining m-1 subgroups as training subgroups; (2) training a GMNB classifier for the fold using the copy number variation data for the candidate segment in the m-1 training subgroups; and (3) testing the trained GMNB classifier for the fold using the copy number variation data for the excluded testing subgroup resulting in a measure of effectiveness of the trained GMNB classifier for the fold; and (ii) determining a mean measure of effectiveness of the trained GMNB classifier across the folds for the current most relevant subset of the candidate segments, which is referred to as the current measure of effectiveness for the current most relevant subset of the candidate segments; (H) removing one or more of the least relevant candidate segments from the current most relevant subset of the candidate segments changing it into an immediately prior most relevant subset of candidate segments and forming a new current most relevant subset of the candidate segments, and labeling the current measure of effectiveness as the immediately prior measure of effectiveness for the immediately prior most relevant set of candidate segments; and (I) repeating (G) for the new current most relevant subset of the candidate segments to determine a current measure of effectiveness for the current most relevant subset of the candidate segments; where the current measure of effectiveness for the current most relevant subset of the candidate segments is statistically worse than the immediately prior measure of effectiveness for the immediately prior most relevant set of candidate segments, select the immediately prior most relevant set of candidate segments as the plurality of target segments; where the current measure of effectiveness for the current most relevant subset of the candidate segments is statistically better than or statistically the same as the immediately prior measure of effectiveness for the immediately prior most relevant set of candidate segments, performing (H) and (I) until the current measure of effectiveness for the current most relevant subset of the candidate segments is worse than the immediately prior measure of effectiveness for the immediately prior most relevant set of candidate segments.

In some embodiments, the system further comprises one or more of: a graphical user interface configured to display an identification of the subject as a candidate for treatment with a double strand break-inducing agent, to display a recommendation of treatment with a double strand break-inducing agent chemotherapy or immunotherapy for the subject, or both; storage configured to store data identifying the subject as a candidate for treatment with a double strand break-inducing agent, to store data including a recommendation of treatment with a double strand break-inducing agent chemotherapy or immunotherapy for the subject, or both; or a communication module configured to send an electronic communication including an identification of the subject as a candidate for treatment with a double strand break-inducing agent, configured to send an electronic communication including a recommendation of treatment with a double strand break-inducing agent chemotherapy or immunotherapy for the subject, or both.

In some embodiments, the training data for the trained classifier comprises a first group of samples with confirmed mutations in the BRCA1 gene and/or the BRCA2 gene, and a second group of samples that are confirmed negative for mutations in the BRCA1 gene and the BRCA2 gene and confirmed negative for mutations in any double strand break repair genes. In some embodiments, the training data for the trained classifier further comprises a third group of samples with mutations in one double strand break repair gene.

In some embodiments, the trained classifier is a geometric mean naïve Bayesian classifier.

In some embodiments, the plurality of target genes are selected from Table 2. In some embodiments, at least some of the plurality of genes are selected from Table 2.

In some embodiments, the sample is a tumor sample. In some embodiments, the sample is a solid tumor sample. In some embodiments, the sample is a tissue biopsy of the cancer or a liquid biopsy.

In some embodiments, the sample comprises one or more of a tissue sample, a body fluid, or cell-free DNA. In some embodiments, the tissue sample includes surgical resection tissue or biopsy tissue from a tumor. In some embodiments, the sample comprises a body fluid and the body fluid includes one or more of amniotic fluid, aqueous humor, bile, blood, blood plasma, a component of blood, cerebrospinal fluid, cerumen, earwax, cowper's fluid, pre-ejaculatory fluid, chyle, chyme, stool, female ejaculate, interstitial fluid, intracellular fluid, lymph, menses, breast milk, mucus, pleural fluid, peritoneal fluid, pus, saliva, sebum, semen, serum, sweat, synovial fluid, tears, urine, vaginal lubrication, vitreous humor, or vomit. In some embodiments, the sample comprises bone marrow cells or peripheral blood cells.

In some embodiments, the cancer is a breast cancer. In some embodiments, the cancer is an ovarian cancer. In some embodiments, the cancer is one or more of lymphoma, leukemia, or a solid tumor.

### BRIEF DESCRIPTION OF THE DRAWINGS

FIG. 1 is a flow chart of a method for classifying a sample as having homologous recombination deficiency (HRD) or DNA double strand break repair deficiency (DSB repair deficiency) or as not having HRD or DSB repair deficiency, or for classifying a sample as having a BRCA1/2 mutation or not having a BRCA1/2 mutation, in accordance with some embodiments.

FIG. 2A is a flow chart of a method for determining target segments and a trained classifier for methods in accordance with some embodiments.

FIG. 2B is a continuation of the flow chart of FIG. 2A.

FIG. 3 is a graph of three probability estimates for d=10 and r=0.5 demonstrating how Geometric Mean Naïve Bayesian probability estimates as described herein closely approximate the true probability for an example probability distribution in contrast with the simple Naïve Bayesian probability estimates, which change too steeply near the boundary due to the independence assumption.

FIG. 4A schematically depicts a networked computing system for implementing some aspects in accordance with some embodiments.

FIG. 4B schematically depicts a computing system including modules for implementing aspects in accordance with some embodiments.

FIG. 5 schematically depicts a computing system or computing device for implementing some aspects in accordance with some embodiments.

FIG. 6 depicts a receiver operating characteristic (ROC) curve for prediction of HRD in samples with BRCA1/2 mutations. The area under the curve (AUC) of 0.984 is obtained using 31 samples with pathogenic BRCA1/2 mutations and 84 samples with no evidence of mutations in any of the DSB genes. TPF, true positive fraction (sensitivity); FPF, false positive fraction (specificity).

FIG. 7 includes representative example plots of log 2 copy ratio of sequence segments (bins) in one example of breast cancer with BRCA1 mutation (Panel A) and an example of cancer without BRCA mutation (panel B).

FIG. 8 includes box plots showing the HRD scores obtained by machine learning for samples with BRCA1/2 mutations, with DSB mutations other than BRCA1/2, and with no mutation in any DSB genes (DSB-null). There was significant difference (P<0.0001) between the BRCA1/2 mutant group and the other two groups, but there was no significant difference between DSB-mutant group (other than BRCA1/2) and the DSB-null group.

### DETAILED DESCRIPTION OF THE INVENTION

As noted above, different approaches have been explored to evaluate HRD including whole-genome sequencing (WGS), comparative genomic hybridization (CGH), and expression profiling and functional assays. Most of these approaches are cumbersome and introduce numerous new variables that complicate the potential of reproducibility and reliability of such assays.

In contrast, some methods and system described herein employ copy number variation (CNV) generated from routine targeted Next Generation Sequencing (NGS) for selected gene sequences along with machine learning for the prediction of or classification of a sample as having a high level of HRD and therefore having a same biological abnormality as a level of HRD associated with a mutation of a BRCA1 or BRCA2 gene (BRCA1/2 gene) irrespective of the gene involved. Some methods and systems described herein employ CNV generated from routine targeted NGS for selected gene sequences along with machine learning for the prediction of or classification of a sample as having a mutation of a BRCA1/2 gene or a level of HRD similar to that caused by a mutation of the BRCA1/2 gene irrespective of the mutated gene involved. Some methods and systems described herein employ CNV generated from routine targeted NGS for selected gene sequences along with machine learning for prediction of or classification of a sample as having a mutation of the BRCA1/2 gene or a genomic structural abnormality biologically similar to a mutation of the BRCA1/2 gene indicating a similar level of HRD as that caused by a mutation of the BRCA1 or the BRCA2 gene irrespective of the mutated gene involved. These methods and systems do not require whole-genome sequencing, comparative genomic hybridization, or expression profiling and functional assays, which reduces complexity and can increase reliability relative to prior methods and systems.

Some methods and systems identify a patient likely to benefit from a treatment with a therapeutic agent directed to addressing DNA double strand break (DSB) repair deficiency. Some methods and systems identify a patient likely to benefit from a treatment with a double strand break-inducing agent.

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

The term “about” is used herein to mean within the typical ranges of tolerances in the art. For example, “about” can be understood as about 2 standard deviations from the mean.

According to certain embodiments, about means+10%. According to certain embodiments, about means+5%. When about is present before a series of numbers or a range, it is understood that “about” can modify each of the numbers in the series or range.

The term “at least” prior to a number or series of numbers (e.g. “at least two”) is understood to include the number adjacent to the term “at least”, and all subsequent numbers or integers that could logically be included, as clear from context. When at least is present before a series of numbers or a range, it is understood that “at least” can modify each of the numbers in the series or range.

As used herein, “up to” as in “up to 10” is understood as up to and including 10, i.e., 0, 1, 2, 3, 4, 5, 6, 7, 8, 9, or 10.

Ranges provided herein are understood to include all individual integer values and all subranges within the ranges.

The term “administer” as used herein means to give or to apply. The term “administering” as used herein includes in vivo administration, as well as administration directly to tissue ex vivo. “Administering” may be accomplished by any route as disclosed below.

The term “allele” as used herein refers to any of one or more alternative forms of a given gene. Generally the alleles of a given gene are concerned with the same trait or characteristic, but the product or function coded for by a particular allele may differ, quantitatively and/or qualitatively, from that coded for by other alleles of that gene. A “wild-type allele” is one which codes for a particular phenotypic characteristic found in the wild type strain of a given organism.

The term “allelic imbalance” or “Al” as used herein refers to an imbalance in paternal and maternal alleles with or without changes in the overall copy number of that region.

Characteristic for HRD is Al at the telomeric end of a chromosome (TAI).

The term “aneuploidy” herein refers to an imbalance of genetic material caused by a loss or gain of a whole chromosome, or part of a chromosome.

The terms “chromosomal aneuploidy” and “complete chromosomal aneuploidy” herein refer to an imbalance of genetic material caused by a loss or gain of a whole chromosome, and includes germline aneuploidy and mosaic aneuploidy.

The terms “partial aneuploidy” and “partial chromosomal aneuploidy” herein are used interchangeably to refer to an imbalance of genetic material caused by a loss or gain of part of a chromosome, e.g., partial monosomy and partial trisomy, and encompass imbalances resulting from translocations, deletions and insertions.

As used herein, the term “base pair” or “bp” refers to a unit consisting of two nucleobases bound to each other by hydrogen bonds. Generally, the size of an organism's genome is measured in base pairs because DNA is typically double stranded. However, some viruses have single-stranded DNA or RNA genomes.

The term “biomarker” (or “biosignature”) as used herein refers to peptides, proteins, nucleic acids, antibodies, genes, metabolites, or any other substances used as indicators of a biologic state. In some embodiments, the biomarker(s) is the copy number of genes, which is (are) altered in diseased samples compared to a reference sample. A biomarker or biosignature is a characteristic that is measured objectively and evaluated as a cellular or molecular indicator of normal biologic processes, pathogenic processes, or pharmacologic responses to a therapeutic intervention.

As used herein, the terms “cell-free DNA” and “cfDNA” interchangeably refer to DNA fragments that circulate in a subject's body (e.g., bloodstream) and originate from one or more healthy cells and/or from one or more cancer cells. These DNA molecules are found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject, and are believed to be fragments of genomic DNA expelled from healthy and/or cancerous cells, e.g., upon apoptosis and lysis of the cellular envelope. The cell-free DNA can be in the form of microvesicles, exosomes, apoptotic bodies, or DNA-protein complexes.

The term “copy number” as used herein refers to the number of copies of a given gene product per copy of that gene or the number of copies of a given gene product per cell.

The abbreviation “CNV” denotes Copy Number Variation.

The term “decrease” or “reduce” and their various grammatical forms is used herein to refer to a diminution, a reduction, an attenuation or abatement of the degree, intensity, extent, size, amount, density or number of occurrences, events or characteristics. As used herein, the term “increase” and its various grammatical forms refers to becoming or making greater in size, amount, intensity, or degree, such as a n increase in the number of occurrences, events or characteristics.

As used herein, the term “derived from” refers to any method for receiving, obtaining, or modifying something from a source of origin.

The term “DNA homology” as used herein refers to the degree of similarity (“relatedness”) between base sequences in different DNA molecules (or in different parts of the same DNA molecule; two DNA molecules which are 100% homologous have identical sequences of nucleotides.

The abbreviation “DSB” denotes DNA Double Stranded Break.

The term “effective amount,” is used herein to include the amount of an agent that, when administered to a patient for treating a subject having a disease, e.g., cancer, is sufficient to effect treatment of the disease (e.g., by diminishing, ameliorating or maintaining the existing disease or one or more symptoms of disease or its related comorbidities). The “effective amount” may vary depending on the agent, how it is administered, the disease and its severity and the history, age, weight, family history, genetic makeup, stage of pathological processes, the types of preceding or concomitant treatments, if any, and other individual characteristics of the patient to be treated. An effective amount includes an amount that results in a clinically relevant change or stabilization, as appropriate, of an indicator of a disease or condition. The term includes prophylactic or preventative amounts of the compositions of the described invention. In prophylactic or preventative applications of the described invention, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

As used herein, the term “exome sequencing” or grammatical variations thereof refers to sequencing of all or select regions of protein-coding regions of genes, e.g., exons. Exome DNA is enriched by isolation of exon DNA by one or more exon-specific markers, e.g., histone modifications, e.g., H3K9ac and H3K4me3.

The term “gene” as used herein refers to a region of DNA that controls a discrete hereditary characteristic, usually corresponding to a single protein or RNA. This definition includes the entire functional unit, encompassing coding DNA sequences, noncoding regulatory DNA sequences, and introns.

As used herein, the term “gene fusion” refers to the product of large scale chromosomal aberrations resulting in the creation of a chimeric protein. These expressed products can be non-functional, or they can be highly over or underactive. This can cause deleterious effects in cancer such as hyper-proliferative or anti-apoptotic phenotypes

As used herein, the terms “genomic alteration,” “mutation,” and “variant” refer to a detectable change in the genetic material of one or more cells. A genomic alteration, mutation, or variant can refer to various type of changes in the genetic material of a cell, including changes in the primary genome sequence at single or multiple nucleotide positions, e.g., a single nucleotide variant (SNV), a multi-nucleotide variant (MNV), an indel (e.g., an insertion or deletion of nucleotides), a DNA rearrangement (e.g., an inversion or translocation of a portion of a chromosome or chromosomes), a variation in the copy number of a locus (e.g., an exon, gene, or a large span of a chromosome) (e.g., copy number variation “CNV”), a partial or complete change in the ploidy of the cell, as well as in changes in the epigenetic information of a genome, such as altered DNA methylation patterns. In some embodiments, a mutation is a change in the genetic information of the cell relative to a particular reference genome, or one or more ‘normal’ alleles found in the population of the species of the subject.

For instance, mutations can be found in both germline cells (e.g., non-cancerous, ‘normal’ cells) of a subject and in abnormal cells (e.g., pre-cancerous or cancerous cells) of the subject. As such, a mutation in a germline of the subject (e.g., which is found in substantially all ‘normal cells’ in the subject) is identified relative to a reference genome for the species of the subject. However, many loci of a reference genome of a species are associated with several variant alleles that are significantly represented in the population of the subject and are not associated with a diseased state, e.g., such that they would not be considered ‘disease-causing.’ By contrast, in some embodiments, a mutation in a cancerous cell of a subject can be identified relative to either a reference genome of the subject or to the subject's own germline genome. In certain instances, identification of both types of variants can be informative. For instance, in some instances, a mutation that is present in both the cancer genome of the subject and the germline of the subject is informative for precision oncology when the mutation is a so-called ‘driver mutation,’ which contributes to the initiation and/or development of a cancer. However, in other instances, a mutation that is present in both the cancer genome of the subject and the germline of the subject is not informative for precision oncology, e.g., when the mutation is a so-called ‘passenger mutation,’ which does not contribute to the initiation and/or development of the cancer. Likewise, in some instances, a mutation that is present in the cancer genome of the subject but not the germline of the subject is informative for precision oncology, e.g., where the mutation is a driver mutation and/or the mutation facilitates a therapeutic approach, e.g., by differentiating cancer cells from normal cells in a therapeutically actionable way. However, in some instances, a mutation that is present in the cancer genome but not the germline of a subject is not informative for precision oncology, e.g., where the mutation is a passenger mutation and/or where the mutation fails to differentiate the cancer cell from a germline cell in a therapeutically actionable way.

As used herein, the term “in combination with,” is not intended to imply that the therapy or the therapeutic agents must be administered at the same time and/or formulated for delivery together, although these methods of delivery are within the scope described herein.

The therapeutic agents can be administered concurrently with, prior to, or subsequent to, one or more other additional therapies or therapeutic agents.

The abbreviation “HRD” denotes Homologous Recombination Deficiency.

The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (e.g., choices of drug treatment or administration regimes). In some embodiments, the biomarker, meaning one or more genes, has/have an altered copy number, e.g., indicator, compared to a reference sample. In some embodiments, the indicator is that the copy number of one or more genes is decreased compared to a reference sample. In some embodiments, the indicator is that the copy number of one or more genes is increased compared to a reference sample. In some embodiments, the reference sample may be a pooled reference sample

The term “inhibitor” as used herein refers to a molecule that reduces the amount or rate of a process, stops the process entirely, or that decreases, limits, or blocks the action or function thereof. Enzyme inhibitors are molecules that bind to enzymes thereby decreasing enzyme activity. Inhibitors may be evaluated by their specificity and potency.

As used herein, the term “insertions and deletions” or “indels” refers to a variant resulting from the gain or loss of DNA base pairs within an analyzed region.

The term “large-scale state transitions or “LSTs” as used herein refers to chromobomal breaks between adjacent regions of at least 10 mb.

As used herein, the term “liquid biopsy” sample refers to any sample taken from a subject, which can reflect a biological state associated with the subject, and that includes cell-free DNA. Examples of sources of liquid biopsy samples include, but are not limited to, blood, whole blood, plasma, serum, urine, cerebrospinal fluid, fecal, saliva, sweat, tears, pleural fluid, pericardial fluid, or peritoneal fluid of the subject. A liquid biopsy sample can include any tissue or material derived from a living or dead subject. A liquid biopsy sample can be a cell-free sample. A liquid biopsy sample can comprise a nucleic acid (e.g., DNA or RNA) or a fragment thereof.

As used herein, the term “locus” refers to a position (e.g., a site) within a genome, e.g., on a particular chromosome. In some embodiments, a locus refers to a single nucleotide position, on a particular chromosome, within a genome. In some embodiments, a locus refers to a group of nucleotide positions within a genome. In some instances, a locus is defined by a mutation (e.g., substitution, insertion, deletion, inversion, or translocation) of consecutive nucleotides within a cancer genome. In some instances, a locus is defined by a gene, a sub-genic structure (e.g., a regulatory element, exon, intron, or combination thereof), or a predefined span of a chromosome. Because normal mammalian cells have diploid genomes, a normal mammalian genome (e.g., a human genome) will generally have two copies of every locus in the genome, or at least two copies of every locus located on the autosomal chromosomes, e.g., one copy on the maternal autosomal chromosome and one copy on the paternal autosomal chromosome. As used herein, the term “allele” refers to a particular sequence of one or more nucleotides at a chromosomal locus. In a haploid organism, the subject has one allele at every chromosomal locus. In a diploid organism, the subject has two alleles at every chromosomal locus.

“Loss of heterozygocity” or “LOH” refers to a situation where one of the two alleles that was originally present in the cell is lost.

The term “nucleic acid” as used herein refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group.

The term “nucleoside” as used herein refers to a compound consisting of a purine or pyridine base covalently linked to a pentose—usually ribose (in ribonucleosides) or 2-deoxyribose (in deoxyribonucleosides. Ribonucleotides containing the bases adenine, guanine, cytosine, uracil, tymine and hyoxanthine are called, respectively, adenosine, guanosine, cytidine, uridine, thymidine, and inosine. The corresponding deoxyribonucleodies are called doxyadenosine, deoxyguanosine, etc.

The term “nucleotide” as used herein refers to a nucleoside in which the sugar carries one or more phosphate groups; nucleotides are the subunits of nucleic acids.

The term “pharmaceutical composition” is used herein to refer to a composition that is employed to prevent, reduce in intensity, cure or otherwise treat a target condition or disease.

The terms “formulation” and “composition” are used interchangeably herein to refer to a product of the described invention that comprises all active and inert ingredients.

The term “pharmaceutically acceptable,” is used to refer to the carrier, diluent or excipient being compatible with the other ingredients of the formulation or composition and not deleterious to the recipient thereof. The carrier must be of sufficiently high purity and of sufficiently low toxicity to render it suitable for administration to the subject being treated.

The carrier further should maintain the stability and bioavailability of an active agent. For example, the term “pharmaceutically acceptable” can mean approved by a regulatory agency of the Federal or a state government or listed in the U.S. Pharmacopeia or other generally recognized pharmacopeia for use in animals, and more particularly in humans.

The term “quantitative PCR” (or “qPCR”), also called “real time-PCR” or “quantitative real-time PCR” refers to a polymerase chain reaction-based technique that couples amplification of a target DNA sequence with quantification of the concentration of that DNA species in the reaction.

The term “recombination” as used herein refers to a process in which one or more nucleic acid molecules are re-arranged to generate new combinations or sequences of genes, alleles, or other nucleotide sequences. It may involve, e.g., the physical exchange of material between two molecules, the integration of two molecules to form a single molecule, the inversion of a segment within a molecule, etc. There are several categories of recombination, including general recombination (“homologous recombination”), site-specific recombination, and transpositional recombination. General recombination (homologous recombination) occurs only between two sequences which have fairly extensive regions of homology; the sequences may be in different molecules or in different regions of the same molecule. In addition to homology, another requirement for homologous recombination is the availability of single-stranded DNA in at least one of the molecules.

The term “recombination repair” as used herein refers to a DNA repair process which can repair a DNA lesion, e.g., thymine dimers or double-strand breaks. In E. coli, during replication of a double-stranded DNA molecule containing a thymine dimer, the DNA polymerase stops at the site of the dimer; DNA synthesis may be re-initiated by primer synthesis at a site beyond the lesion, leaving a gap in the daughter strand opposite the dimer in the damaged parent strand. This gap can be filled by recombination between the gapped daughter strand and an undamaged homologous region of the strand complementary to the damaged parent strand. The resulting gap in the homologous parent strand can then be filled by repair synthesis, its daughter strand acting as template. According to one model (Meselson, M S and Radding, CM, PNAS (1975) 72: 358-61), recombination is initiated by a nick in one strand of a donor duplex. The 3′ end generated by the nick serves as a primer for DNA synthesis and is extended, displacing the 5′ end which then invades a homologous region of the recipient duplex to generate a D loop. The donor strand is ligated in place, and the D loop is degraded. The heteroduplex region can be extended by further nuclease action on the recipient duplex and DNA polymerase action on the donor duplex. Ligation completes the structure. Genes that have been described in daughter-strand gap repair include recA, ruv, lexA and recF. In E. coli, the inducible double-strand break repair system requires the presence of at least one other dsDNA molecule homologous to the damaged molecule. This system requires, e.g., recA and recN(SOS genes) (See also Szostak, J W et al. Cell (1983) 33 (1): 25-35).

The term “ROC” denotes a receiver operating characteristic curve. A ROC curve is a graph showing the performance of a classification model at all classification thresholds. It plots two parameters: true positive versus false positive. The area under the ROC curve is the AUC value. An AUC value of 0.5 is equivalent to a random prediction, and a value of 1.0 is equivalent to a perfect prediction. In general, an AUC of 0.7-0.8 is considered acceptable, 0.8 to 0.9 is considered excellent, and more than 0.9 is considered outstanding.

As used herein, the term “sample” refers to a tissue sample (e.g., cancer biopsy) such as a small intestine, colon sample, or surgical resection tissue comprising cancerous issue or a body fluid, e.g. whole blood. plasma, serum saliva, urine, stool (feces) tears, and any other bodily fluid. The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle. chyme, stool female ejaculate, Interstitial fluid, intracellular fluid, lymph, menses. breast milk, mucus, pleural fluid, peritoneal fluid, pus, saliva, sebum, semen, serum, sweat, synovial fluid, tears, urine, vaginal lubrication, vitreous humor, vomit). I some embodiments, the sample includes bone marrow cells or peripheral blood cells. In some embodiments, the sample is a biopsy from a tumor (e.g, breast, ovary, endometrial, lung, colorectal, and pancreas). In some embodiments, the sample is cell-free DNA.

As used herein, the term “sequence reads” or “reads” refers to nucleotide sequences produced by any nucleic acid sequencing process described herein or known in the art. Reads can be generated from one end of nucleic acid fragments (“single-end reads”) or from both ends of nucleic acid fragments (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore® sequencing methods and associated devices provided by Oxford Nanopore Technology PLC of Oxford, UK, for example, can provide sequence reads that can vary in size from tens to hundreds to thousands of base pairs.

Illumina® parallel sequencing methods and associated devices provided by Illumina Inc. of San Diego, CA, for example, can provide sequence reads that do not vary as much, for example, most of the sequence reads can be smaller than 200 bp. A sequence read (or sequencing read) can refer to sequence information corresponding to a nucleic acid molecule (e.g., a string of nucleotides). For example, a sequence read can correspond to a string of nucleotides (e.g., about 20 to about 150) from part of a nucleic acid fragment, can correspond to a string of nucleotides at one or both ends of a nucleic acid fragment, or can correspond to nucleotides of the entire nucleic acid fragment. A sequence read can be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

As used herein, the term “single nucleotide variant” or “SNV” refers to a substitution of one nucleotide to a different nucleotide at a position (e.g., site) of a nucleotide sequence, e.g., a sequence read from an individual. A substitution from a first nucleobase X to a second nucleobase Y may be denoted as “X>Y.” For example, a cytosine to thymine SNV may be denoted as “C>T.”

The term “split gene” as used herein refers to a structural gene (encoding e.g., a protein, rRNA or tRNA) that contains one, several or many specific sequences of nucleotides (intervening sequences; introns) which, although represented in the primary RNA transcript of the gene, are absent from the mature RNA molecule (mRNA, tRNA, etc.) and hence do not encode any part of the gene product. Thus, maturation of the primary RNA transcript of a split gene must involve a process of splicing in which sequences corresponding to introns are deleted (“spliced out”) and the remaining sequences (“exons”) are joined together. In some cases (“alternative splicing”), a given gene can be spliced in different ways to yield different versions of the encoded product, i.e., splicing can give rise to different combinations of exons, producing correspondingly different coding sequences.

The term “signature” as used herein refers to a specific and complex combination of biomarkers that reflect a biological state. In some embodiments, the signature comprises a copy number variation indicative of disease or progression of a disease, e.g., cancer or the progression of the cancer.

The terms “subject”, “animal,” and “patient,” are used interchangeably herein to refer, for example, and without limitation, to humans and non-human vertebrates such as wild, domestic and farm animals. According to some embodiments, the terms “animal,” “patient,” and “subject” may refer to humans. According to some embodiments, the terms “animal,” “patient,” and “subject” may refer to non-human mammals. According to some embodiments, the terms “animal,” “patient,” and “subject” may refer to any or combination of: dogs, cats, pigs, cows, horses, goats, sheep or other domesticated non-human mammals.

The term a “subject in need” of treatment for a particular condition can be a subject having that condition, diagnosed as having that condition, or at risk of developing that condition.

The term “therapeutic agent” as used herein refers to a drug, molecule, composition or other substance that provides a therapeutic effect. The term “active agent” as used herein refers to the ingredient, component or constituent of the compositions of the present invention responsible for the intended therapeutic effect. The terms “therapeutic agent” and “active agent” are used interchangeably herein.

The term “DSB-inducing agent” refers to a therapeutic agent that induces DNA double strand breaks in the genome. In some embodiments, the DSB-inducing agent is high dose platinum-based alkvlating chemotherapy. platinum compounds, thiotepa, cyclophosphamide, iphosphamide, nitrosureas, nitrogen mustard derivatives, mitomycins, epipodophyllotoxins, camptothecins, anthracyclines, poly(ADP-ribose) polymerase (PARP) inhibitors, ionizing radiation, ABT-888, olaparib (AZT-2281), gemcitabine, CEP-9722, AG014699, AG014699 with Temozolomide, and BSI-201.

In some embodiments. the therapeutic agent is one or more platinum compounds (e.g., isplatin. carboplatin, oxaliplatin, nedaplatin, and lobapiatin); ethylenimines (e.g., thiotepa and hexamethylmelamine); alkylsulfonates (e.g., busulfan); nitrosureas (e.g. carmustine (BCNU), lomustine (CCNU), semustine (methyl-CCNU), nimustine (ACN U), fotemustine, and streptozotocin); nitrogen mustard derivatives (e.g., mechlorethamine, cyclophosphamide, chloranbucil, melphalan, and ifosfamide): mitomycin; hydrazines and triazines (e.g. altretamine, procarbazine, dacarbazine and temozolomide); epipodophyllotoxins (e.g. etoposide and teniposide); camptothecins (e.g., topotecan, irinotecan. belotecan, and trastuzumab deruxtecan); anthracyclines (e.g., daunorubicin, doxorubicin (adrianmycin), doxorubicin liposomal, epirubicin, idarubicin, and valrubicin); ionizing radiation: antimetabolites (cg. gemcitabine, hydroxyurea, methotrexate, mercaptopurine, Cladribine, pralatrexate, fludarabine, pemetrexed. thioguanine, cytarabine liposomal, decitabine, clofarabine, nelarabine, fluorouracil and capecitabine); vinca alkaloids(e.g., vinblastine (VBL), vinorelbine (VRL) vincristine (VCR) and vindesine (VDS)); taxanes (e.g., docetaxel, oraxol, paclitaxel, paclitaxel/encequidar, taxol, and taxotere); and poly(ADP-ribose) polymerase (PARP) inhibitors, including PARP-1 and PARP-2 inhibitors (e.g., 5-aminoisoquinoline; 3-methyl-5-aminoisoquinoline; 3-aminobenzamide; 5-iodo-6-amino-1,2-benzopyrone; 3,4-dihydro-5[4-(1-piperidinyl)butoxy]-1(2H)-isoquinoline; 1,5-dihydroxyisoquinoline;-aza-5[H]-phenanthridin-6-ones; 6(5H)-phenanthridinone: 4-amino-1,8-naphthalimide; 8-hydroxy-2-methylquinazoline-4-one: N-(6-oxo-5,6-dihydrophenanthridin-2-yl)-N,N-dimethylacetamide; indeno-isoquinolinone; 5-chloro-2-[3-(4-phenyl-3,6-dihydro-1(2H)-pyridinyl)propyl]4(3H)-quinazolinone; 1-piperazineacetamide, 4-1-(6-amino-9H-purin-9-yl)-1-deoxy-β-D-ribofuranuronic-N-(2,3-dihydro-1H-isoindol-4-yl)-1-one: thieno[2,3-c]isoquinolin-5-one; 2-dimethylaminomethyl-4-1-thieno [2,3-c]isoquinolin-5-one: 4-hydroxyquinazoline: nicotinamide: minocycline: 2-methyl-3,5,7,8-tetrahydrothiopyrano[4,3-d]pyrimidine-4-one; 3-(4-chiorophenyl)quinoxaline-5-carboxamide; benzamide; N-(6-oxo-5,6-dihydrophenanthridin-2-L)-2-(N,N-dimethylaminoacetamido: AG014699; AG-14361; 2-[(R)-2-methylpyrrolidin-2-yl]-1H-benzimidazole-4-carboxamide; 4-[3-(4-cyclopropanecarbonylpiperazine-1-carbonyls)-4-fluorobenzyl-2H-phthalazin-1-one; BSI-401; BSI-201; CEP-8983; CEP-9722: GPI-21016; GPI 16346; GPI 18180; GPI 6150; GPI 18078: GPI 6000; 2-aminothiazole analogues: quinoline-8-carboxamides; 2—and 3-substituted quinoline-8-carboxamides; 2-methylquinoline-8-carboxamide; 2-(1-propylpiperidin-4-yl)-1H-benzonidazol-4-carboxamide: aminoethyl pyrrolo dihydroisoquinoline; imidazo quinolinone and derivatives thereof; imidazopyridine and derivatives thereof; isoquinolinedione and derivatives thereof; 2-f45-Methyl-1H-imidazol-4-yl)-piperidin-1-yl-4,5-dihydro-imidazo[4,5,1-i,j]quinolin-6-one; 2-(4-pyridin-2-yl-phenyl)-4,5-dihydro-imidazo[4,5,1-i,j]quinolin-6-one; 6-chloro-8-hydroxy-2,3˜dimethyl-imidazo-[1,2α]pyridine; 4-(1-methyl-1H-pyrrol-2-ylmethylene)-4H-isoquinolin-1,3-dione; E7016; 2-[methoxycarbonyl(4-methoxyphenyl)methylsulfanyl]-1-benzimidazole-4-carboxylic Acid Amide; 4-carboxamicdobenzirnidazole-2-ylpyrazine; (tetrahydropyridine nitroxides derivatives; N—-3(4-oxo-3,=4-dihydro-phthalazin-1-yl)phenyl]-4-(morpholin-yl) butanamide methanesulfonate monohydrate; phenanthridinone; 4iodo-3-nitrobenzamide; 2-(4-hydroxyphenyl)-1H-benzimidazole 4-carboxamide; 2-aryl-1H-benzimidazole-4-carboxamides; 2-phenyl benzimidazole-4-carboxamides; phthalazin-1(2H)-one; 3-substituted 4-benzyl-2H-phthalazin--1-ones, ABT-888, Olaparib, niraparib. rucaparib, CEP-9722, AG014699, AG014699 with Temozolomide, and BSI-201 and derivatives).

The term “therapeutic component” as used herein refers to a therapeutically effective dosage (i.e., dose and frequency of administration) that eliminates, reduces, or prevents the progression of a particular disease manifestation in a percentage of a population.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. The term “treat” or “treating” as used herein further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

Treatment includes eliciting a clinically significant response without excessive levels of side effects. Treatment also includes prolonging survival as compared to expected survival if not receiving treatment.

The term “unique molecule identifiers” or “UMIs” as used herein refers to a type of molecular barcoding that provides error correction and increased accuracy during sequencing.

These molecular barcodes are short sequences used to uniquely tag each molecule in a sample library.

Methods

In some embodiments, the methods disclosed herein classify a sample from a patient who has cancer as having a high level of homologous recombination deficiency (HRD) and therefore having a same biological abnormality as a level of HRD associated with a mutation of a BRCA1 or a BRCA2 gene (BRCA1/2 gene) irrespective of the mutated gene involved, or as not having a high level of HRD and therefore not having the same biological abnormality as the level of HRD associated with a mutation of the BRCA1/2 gene, irrespective of the mutated gene involved. In some embodiments, the methods disclosed herein classify a sample from a patient who has cancer as having a mutation of a BRCA1/2 gene or a level of HRD similar to that caused by a mutation of a BRCA1/2 gene, irrespective of the mutated gene involved, or as not having a mutation of the a BRCA1/2 gene or a level of HRD similar to that caused by a mutation of a BRCA1/2 gene, irrespective of the mutated gene involved.

In some embodiments, the methods disclosed herein classify a sample from a patient who has cancer as having a mutation of a BRCA1/2 gene or genomic structural abnormalities similar to a mutation of a BRCA1/2 gene, irrespective of the mutated gene involved, indicating a similar level of HRD as that caused by a mutation of the BRCA1/2 gene, or as not having a mutation of the BRCA1/2 gene or genomic structural abnormalities similar to a mutation of the BRCA1/2 gene.

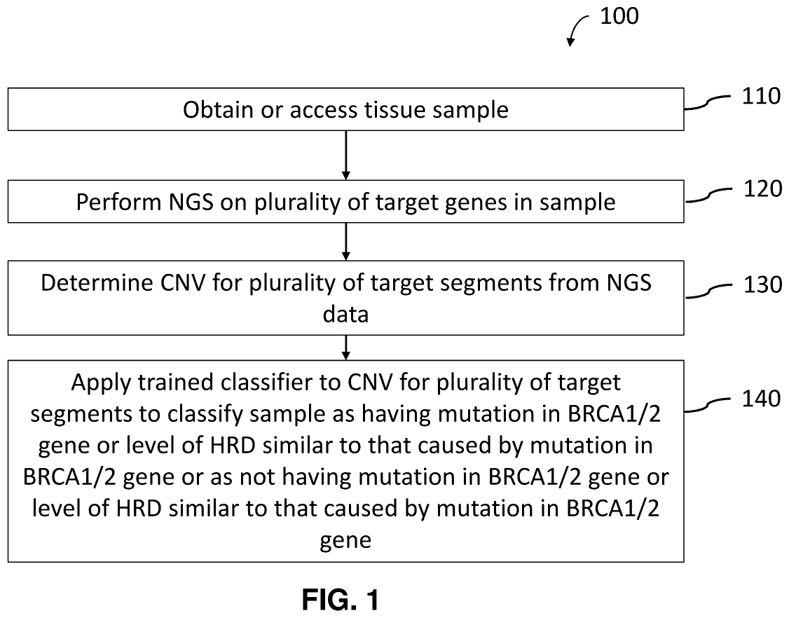

An embodiments of a method is illustrated in FIG. 1. Method 100 includes obtaining or accessing a sample from a patient 110. In some embodiments, the sample is a tissue sample. In some embodiments, the sample is a solid tissue sample. In some embodiments, the sample is a tissue sample from a tumor. In some embodiments, the sample is a solid tissue sample from a tumor. In some embodiments, the sample includes bone marrow cells, peripheral blood cells, lymph node cells, cfDNA or any combination of the aforementioned.

The method also includes performing Next Generation Sequencing (NGS) to obtain sequence information for a plurality of targeted genes 120. In some embodiments, the plurality of targeted genes includes at least 275 genes. In some embodiments, the plurality of targeted genes includes at least 300 genes. In some embodiments, the plurality of targeted genes includes at least 350 genes. In some embodiments, the plurality of targeted genes includes at least 400 genes. In some embodiments, the plurality of targeted genes includes at least 420 genes. In some embodiments, the plurality of targeted genes includes at least 500 genes. In some embodiments, the plurality of targeted genes includes at least 600 genes. In some embodiments, the plurality of targeted genes includes at least 700 genes. In some embodiments, the plurality of targeted genes includes at least 800 genes. In some embodiments, the plurality of targeted genes includes at least 900 genes. In some embodiments, the plurality of targeted genes includes at least 1000 genes, at least 2000 genes, at least 3000 genes, at least 4000 genes, at least 5000 genes, at least 6000 genes, at least 7000 genes, at least 8000 genes, at least 9000 genes, at least 10,000 genes, at least 11,000 genes, at least 12,000 genes, at least 13,000 genes, at least 14,000 genes, at least 15,000 genes, at least 16,000 genes, at least 17,000 genes, at least 18,000 genes, at least 19,000 genes, at least 20,000 genes, at least 21,000 genes, or at least 22,000 genes. In some embodiments, the plurality of number of targeted genes includes between 275 and 22,000 genes. In some embodiments, the plurality of targeted genes includes between 300 and 22,000 genes. In some embodiments, the plurality of number of targeted genes includes between 350 and 22,000 genes. In some embodiments, the plurality of number of targeted genes includes between 400 and 22,000 genes. In some embodiments, the plurality of number of targeted genes includes between 500 and 22,000 genes. In some embodiments, the plurality of number of targeted genes includes between 600 and 22,000 genes. In some embodiments, the plurality of number of targeted genes includes between 700 and 22,000 genes. In some embodiments, the plurality of number of targeted genes includes between 800 and 22,000 genes. In some embodiments, the plurality of number of targeted genes includes between 900 and 22,000 genes. In some embodiments, the plurality of number of targeted genes includes between 1,000 and 22,000 genes.

In some embodiments, the plurality of targeted genes are selected based on relevance to cancer in general, and/or include tumor repair genes, and/or include inherited double strand repair genes. In some embodiments, the plurality of targeted genes includes all of the 434 genes in Table 2 appearing in the Example below. The genes in Table 2 include genes relevant to cancer in general, tumor repair genes, and inherited double strand repair genes. In some embodiments, the plurality of targeted genes includes some of the genes in Table 2. In some embodiments, the plurality of targeted genes includes some of the genes in Table 2 and some genes not listed in Table 2.

As used herein, the term “Next Generation Sequencing” or “NGS” refers to a method of parallel sequencing. For instance, a nucleic acid (e.g., DNA) sample is obtained and prepared into a library (meaning a collection of nucleic acid fragments from the sample). The library is prepared by fragmenting the DNA or RNA sample. Fragmentation can be performed by physical (e.g., sheared by acoustics, nebulization, centrifugal force, needles, or hydrodynamics) or enzymatic (e.g., site-specific or non-specific nucleases) methods. In some embodiments, the fragments are about 200 bp, about 20 bp, about 300 bp, or about 350 bp in length. The DNA or RNA samples are repaired at the ends (e.g., blunt-ended) and then A-tailed (e.g., an adenosine is added to the 3′ end resulting in an overhang). Adapters are ligated to each end. Adapters include sequences, such as barcodes, restriction sites, and primer sequences.

As used herein, the term “coverage” in reference to NGS refers to the average number of reads that align to, or “cover” known reference basis. The sequencing coverage level determines whether variant discovery can be made with a certain degree of confidence at particular base positions. Coverage equals read count multiplied by the read length and divided by the total genome size. At a higher level of coverage, each base is covered by a greater number of aligned sequence reads, and mutations at the base level compared to a reference sample can be determined. In some embodiments, a reference sample may be a pooled reference sample. In some embodiments, the pooled reference sample may be a pooled normal reference sample.

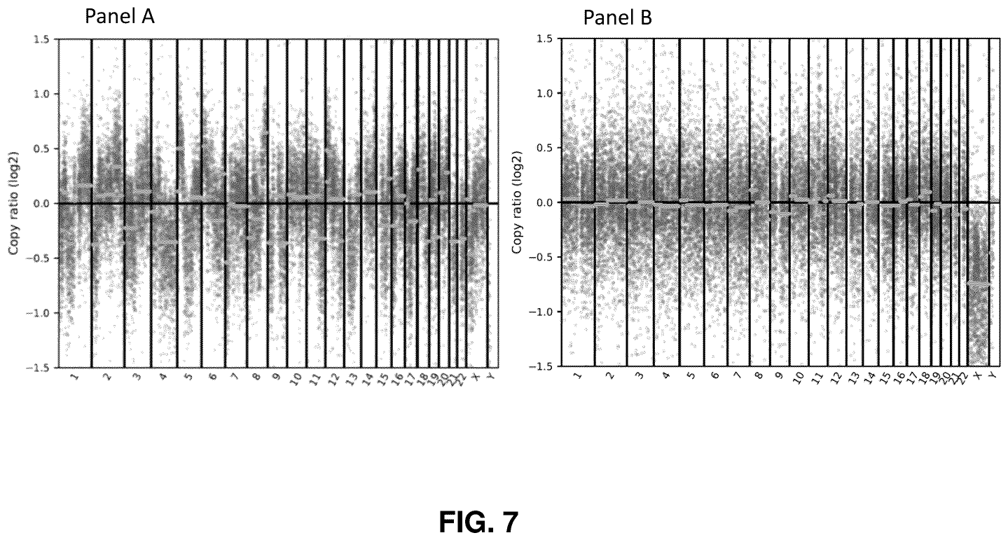

The method also includes determining copy number variation (e.g., bin-level sequence ratios, segment-level sequence ratios, and/or segment-level measures of dispersion) for a plurality of target segments in the NGS data 130. In some embodiments, each segment may be a gene or a fraction of a gene. In some embodiments, the number of segments includes between 5,000 and 100,000 segments. In some embodiments, the number of segments includes between 10,000 and 50,000 segments. In some embodiments, the number of segments includes between 5,000 and 50,000 segments. In some embodiments, the number of segments includes between 10,000 and 40,000 segments. In some embodiments, the number of segments includes between 12,000 and 22,000 segments. In some examples, the number of segments includes at least 5,000; at least 10,000; at least 11,000; at least 12,000; at least 13,000; at least 14,000; at least 15,000; at least 16,000; at least 17,000; at least 18,000; at least 19,000; at least 20,000; at least 21,000; at least 22,000; at least 23,000; at least 24,000; at least 25,000; at least 26,000; at least 27,000; at least 28,000; at least 29,000; at least 30,000; at least 31,000; at least 32,000; at least 33,000; at least 34,000; at least 35,000; at least 36,000; at least 37,000; at least 38,000; at least 39,000; at least 40,000; at least 41,000; at least 42,000; at least 43,000; at least 44,000; at least 45,000; at least 46,000; at least 47,000; at least 48,000; at least 49,000; or at least 50,000 segments. In the Example described below, performing CNV analysis on all available segments of the 434 genes using the CNVkit software package resulted in CNV analysis of 26,940 segments; however, use of only about 16,000 of these segments was sufficient to provide the best sensitivity and specificity. A description of a method for determining the segments to include for best sensitivity and specificity is included below in the section below entitled “Determining Target Segments and Training Classifier.” FIG. 7, which is described below with respect to the Example, includes plots of log 2 of the copy ratio of sequence segments (bins) for a sample from a breast cancer with a BRCA mutation (panel A) and for a sample from a cancer without a BRCA mutation (panel B).

As used herein, the term “copy number variation” or “CNV” refers to a variation in the number of copies of a nucleic acid sequence present in a test sample in comparison with the number of copies of the nucleic acid sequence present in a reference sample or a qualified sample. In certain embodiments, the copy number gain or loss can be covering a range of 100 bp to a significant portion of the chromosome or the entire chromosome. A “copy number variant” refers to a sequence of nucleic acid in which copy-number differences are found by comparison of a level a nucleic acid sequence of interest in a test sample with an expected level of the nucleic acid sequence of interest. For example, the level of the nucleic acid sequence of interest in the test sample is compared to that present in a qualified sample. In some embodiments, the reference sample may be a pooled normal sample. Copy number variants/variations include deletions, including microdeletions, insertions, including microinsertions, duplications, multiplications, and translocations. CNVs encompass chromosomal aneuploidies and partial aneuploidies.

De novo copy number variation can occur both in germline and in somatic cells and is generated most commonly through two mechanisms: (1) nonallelic homologous recombination (NAHR) and (2) nonhomologous end joining (NHEJ or microhomology-mediated end joining (MMEJ). NAHR results from incorrect pairing across large homologous regions resulting in gain or loss of intervening segments. Non homologous end joining commonly occurs during DNA repair or DNA recombination, wherein DNA ends are annealed without sequence homology.

As used herein, the term “germline variants” refers to genetic variants inherited from maternal and paternal DNA. Germline variants may be determined through a matched tumor-normal calling pipeline. A bioinformatics pipeline is a set of complex algorithms used to process sequence data in order to generate a list of variants or assemble a genome(s). As used herein, the term “somatic variants” refers to variants arising as a result of dysregulated cellular processes associated with neoplastic cells, e.g., a mutation. Somatic variants may be detected via subtraction from a matched normal sample.

In some embodiments, the CNV may be obtained as an output of a bioinformatics tool (such as the CNVkit software package, which was developed at the University of California, San Francisco and is downloadable from the github depository). A description of the CVkit software appears in the article “CNVkit: Genome-wide copy number detection and visualization from targeted sequencing” by Talevich et al., which is incorporated by reference herein in its entirety. [Talevich, E., Sham, A. H., Botton, T., & Bastian, B. C. (2014). CNVkit: Genome-wide copy number detection and visualization from targeted sequencing. PLOS Computational Biology; 12(4):e1004873]

As used herein, the term “bin” or grammatical variations thereof refers to a subset of a larger grouping, e.g., a genome. Next generation sequencing calculations are performed by first dividing the genome into small regions (bins), on which the calculations are actually performed. The genome is partitioned into bins with an expected equal number of mappable positions. In some embodiments, the bins divide the genome into small or large regions depending on target regions. For example, on-target and off-target regions may be partitioned into bins ranging as small as 100 bp to as large as 1000 s kb. On-target regions may be partitioned into smaller regions, while the off-target regions may be partitioned into larger regions. Copy number variation is determined by determining the read depth for each region (e.g., on- or off-target regions).

Once the genome is partitioned and the bins are ordered, the sequence reads in each bin can be analyzed to determine “bin-level sequence ratios”. Bin-level sequence ratios can be determined by comparing corresponding bins between the sequence reads in each bin from the sample (e.g., DNA from cancerous cells) and from a reference sample (e.g., DNA from healthy cells). For example, in some embodiments, each respective bin in the plurality of bins represents a corresponding region of a human reference genome, and each respective bin-level sequence ratio in the plurality of bin-level sequence ratios is determined from a sequencing of a sample from a tumor (e.g., a hematologic tumor, a solid tumor, etc.). For example, when the ratio between corresponding bins in the sample and reference is close to 1, the copy number between the sample and reference is similar. When the ratio between corresponding bins in the sample and reference is greater or less than 1, the copy number between the sample and reference is dissimilar.

In some embodiments, copy number variation can be determined at the segment-level by determining the segment-level sequence ratios. Each respective segment in the plurality of segments represents a corresponding region of the human reference genome encompassing a subset of adjacent bins in the plurality of bins, and each respective segment-level sequence ratio in the plurality of segment-level sequence ratios is determined from a measure of central tendency of the plurality of bin-level sequence ratios corresponding to the subset of adjacent bins encompassed by the respective segment.

In some embodiments a plurality of segment-level measures of dispersion are determined for a biological sample and/or a reference sample, where each respective segment-level measure of dispersion in the plurality of segment-level measures of dispersion (i) corresponds to a respective segment in the plurality of segments and (ii) is determined using the plurality of bin-level sequence ratios corresponding to the subset of adjacent bins encompassed by the respective segment. As used herein, the term “dispersion”, “variability”, “scatter”, or “spread” refers to how similar a set of scores (e.g., bin-level sequence ratios or segment-level sequence ratios) are to each other. For example, the more similar the scores are (e.g., the bin-level sequence ratio is close to 1) the lower the measure of dispersion will be.

For example, the less similar the scores are (e.g., the bin-level sequence ratio is greater or less than 1) to each other, the higher the measure of dispersion will be stretched or squeezed a distribution in a dataset is.

In some embodiments, the method includes obtaining a dataset including next generation sequencing data, and determining the copy number variation of one or more genes or gene segments. As used herein, the term “gene segment” refers to a region of the genome, more specifically a region of a gene. For instance, sequence reads are aligned and sequence read depth is determined compared to internal standard number of sequence reads (e.g., sequence reads of a genomic segment directly upstream or downstream of the genomic region of interest). As used herein, the term “read depth” or “depth of coverage” refers to the number of reads of a given nucleotide in an experiment. Most NGS protocols start with a random fragmentation of the genome into short random fragments. These fragments are then sequenced and aligned. This alignment creates a longer contiguous sequence. by tiling of the short sequences. In some embodiments, the CNV is determined by applying a copy number segmentation algorithm to the sequencing data.

In some embodiments, sequence reads are obtained from the sequencing dataset and aligned to a reference sample, generating a plurality of aligned reads and further processed using a copy number variation algorithm, which may be implemented in software (e.g., CNVkit). For instance, the copy number variation algorithm implemented in software (e.g., CNVkit) is used for genomic region binning (e.g., bin values, e.g., defining segment size while partitioning the genome), coverage calculation, bias correction, normalization to a reference pool, segmentation, and/or visualization. Bin values are used to determine bin-level sequence ratios between various genomic segments. In some embodiments, the reference sample is a pooled normal sample.

As used herein, the term “reference allele” refers to the sequence of one or more nucleotides at a chromosomal locus that is either the predominant allele represented at that chromosomal locus within the population of the species (e.g., the “wild-type” sequence), or an allele that is predefined within a reference genome for the species.

As used herein, the term “variant allele” refers to a sequence of one or more nucleotides at a chromosomal locus that is either not the predominant allele represented at that chromosomal locus within the population of the species (e.g., not the “wild-type” sequence), or not an allele that is predefined within a reference genome for the species. As used herein, the term “variant allele fraction,” “VAF,” “allelic fraction,” or “AF” refers to the number of times a variant or mutant allele was observed (e.g., a number of reads supporting a candidate variant allele) divided by the total number of times the position was sequenced (e.g., a total number of reads covering a candidate locus).

As used herein, the term “loss of heterozygosity” refers to the loss of one copy of a segment (e.g., including part or all of one or more genes) of the genome of a diploid subject (e.g., a human) or loss of one copy of a sequence encoding a functional gene product in the genome of the diploid subject, in a tissue, e.g., a cancerous tissue, of the subject. As used herein, when referring to a metric representing loss of heterozygosity across the entire genome of the subject, loss of heterozygosity is caused by the loss of one copy of various segments in the genome of the subject. Loss of heterozygosity across the entire genome may be estimated without sequencing the entire genome of a subject, and such methods for such estimations based on gene panel targeting-based sequencing methodologies are described in the art. Accordingly, in some embodiments, a metric representing loss of heterozygosity across the entire genome of a tissue of a subject is represented as a single value, e.g., a percentage or fraction of the genome. In some cases a tumor is composed of various sub-clonal populations, each of which may have a different degree of loss of heterozygosity across their respective genomes. Accordingly, in some embodiments, loss of heterozygosity across the entire genome of a cancerous tissue refers to an average loss of heterozygosity across a heterogeneous tumor population. As used herein, when referring to a metric for loss of heterozygosity in a particular gene, e.g., a DNA repair protein such as a protein involved in the homologous DNA recombination pathway (e.g., BRCA1 or BRCA2), loss of heterozygosity refers to complete or partial loss of one copy of the gene encoding the protein in the genome of the tissue and/or a mutation in one copy of the gene that prevents translation of a full-length gene product, e.g., a frameshift or truncating (creating a premature stop codon in the gene) mutation in the gene of interest. In some cases a tumor is composed of various sub-clonal populations, each of which may have a different mutational status in a gene of interest. Accordingly, in some embodiments, loss of heterozygosity for a particular gene of interest is represented by an average value for loss of heterozygosity for the gene across all sequenced sub-clonal populations of the cancerous tissue. In other embodiments, loss of heterozygosity for a particular gene of interest is represented by a count of the number of unique incidences of loss of heterozygosity in the gene of interest across all sequenced sub-clonal populations of the cancerous tissue (e.g., the number of unique frame-shift and/or truncating mutations in the gene identified in the sequencing data).

Next Generation Sequencing (NGS) has the potential to introduce several biases into the dataset. Bias in sequencing data can result from chromatin structure, enzymatic cleavage, nucleic acid isolation, PCR amplification, and read mapping effects. Both mechanical and enzymatic methods of fragmenting the genome can result in uneven-sized fragments. For example, heterochromatin (e.g., gene segments without coding regions) is more resistant to shearing by sonication than euchromatin (e.g., gene segments under active transcription) because of the more open configuration of euchromatin making it more vulnerable to shearing. Enzymatic digestion can introduce biases depending on the cleavage enzyme used.

For example, MNase has a preference of digesting AT rich regions. Nucleic acid isolation can be incomplete when some DNA is bound by various polypeptides. PCR amplification can introduce bias because the PCR cycle can prefer some segments over other depending on denature and annealing temperatures, polymerase and buffer. Lastly, the sample data set is mapped on to a reference sample, which can introduce a bias toward the specific sequence of the reference sample. [See Meyer C A, Liu X S. Identifying and mitigating bias in next-generation sequencing methods for chromatin biology. Nat Rev Genet. 2014; 15(11):709-721.]

Bias correction for varied GC content can be determined by fitting a rolling median, then subtracted from the original read depths in a sample to yield corrected estimates.

As used herein, the term “normalization” in next-generation sequencing (NGS) is the process of equalizing the concentration of DNA libraries for multiplexing (e.g., annealing individual barcode sequences to individual fragments.

For instance, CNVkit, uses both on-target reads (e.g., genomic segment of interest) and off-target-reads (e.g., genomic region included in the sequencing dataset not specifically sequenced) to calculate log 2 copy ratios across the genome or select segments of the genome.

On- and off-target locations are separately determined and used to calculate the mean read depth within each segment of interest. On- and off-target depth reads are combined, normalized to a reference sample, corrected for systemic biases to result in final log 2 copy ratios. [See Talevich E, Shain A H, Botton T, Bastian B C. CNVkit: Genome-Wide Copy Number Detection and Visualization from Targeted DNA Sequencing. PLoS Comput Biol. 2016 Apr. 21; 12(4):e1004873. doi: 10.1371/journal.pcbi.1004873]. As used herein, “off-target intervals” refers to nonspecifically captured off-target reads.

In some embodiments, a copy number variation algorithm or software (e.g., CNVkit) corrects biases, e.g., genomic GC content and sequence repeats. Genomic G C rich regions are less accessible to hybridization and are less amenable to amplification during sample preparation. For instance, CNVkit applies a rolling median correction to GC values in both on- and off-target bins. Id.

Some methods include applying a machine-learning trained classifier to the copy number variation results for the plurality of target segments to classify the sample as having a mutation of the BRCA1 or the BRCA2 gene or a level of HRD similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved 140. In some embodiments, a method includes applying a machine-learning trained classifier to the copy number variation results for the plurality of target segments to classify the sample as having a high level of HRD and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved. In some embodiments, a method includes applying a machine-learning trained classifier to the copy number variation results for the plurality of target segments to classify the sample as having a mutation of the BRCA1 or the BRCA2 gene or genomic structural abnormalities similar to a mutation of the BRCA1 or the BRCA2 gene, irrespective of the mutated gene involved, indicating a similar level of HRD as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene.

In some embodiments, the machine-learning trained classifier is a geometric mean naïve Bayesian (GMNB) classifier as described herein.

Determining Target Segments and Training Classifier

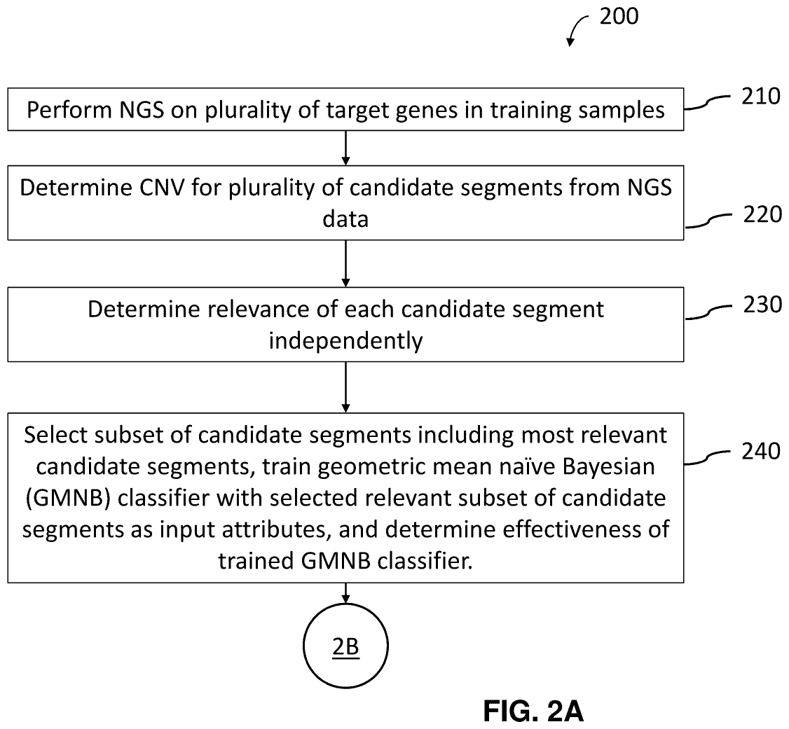

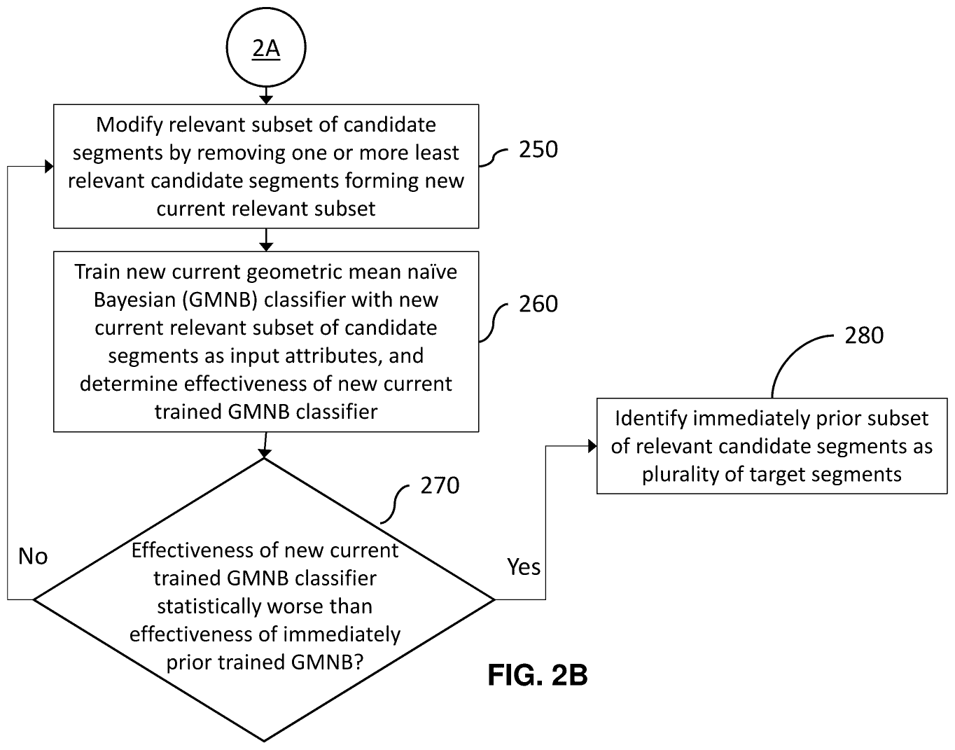

In some embodiments, the classifier is pre-trained and the target segments for which CRV data is used in the classifier have already been identified or pre-selected. In other embodiments, some methods include determining or selecting the target segments for which CRV data will be used in the classifier or to train the classifier. A method 200 for determining target segments in accordance with some embodiments is depicted in FIGS. 2A and 2B.

In some embodiments, training samples, or sequence data from training samples, are employed for training the classifier. In some embodiments, training samples, or sequence data from training samples, are also employed for determining or selecting the target segments to be used in the classifier or for training the classifier. In some embodiments, the training samples include samples known to exhibit HRD (such as samples with mutations in a BRCA1 or BRCA2 gene) and samples that do not exhibit HRD (such as samples with no mutations in a BRCA1 or a BRCA2 gene and confirmed negative for mutations in any of the genes implicated in DSB repair). A description of example training samples is provided below in the Examples.

In some embodiments, NGS is used to obtain sequence data for a plurality of candidate segments in the plurality of target genes in the training samples (210). The candidate segments are candidates for inclusion in the final classifier. Including all of the candidate segments as input to the classifier can lead to problems with noise and overfitting.

In some embodiments, a cross-validation procedure (e.g., a k-fold or leave-one-out cross-validation procedure) is used for selection to determine which of the candidate segments may be most relevant for inclusion in the final classifier. A 10-fold cross-validation procedure is described below for illustrative purposes; however, one of ordinary skill in the art will appreciate that using more or fewer than 10-folds for cross-validation validation could also or alternatively be employed. Another example of k-fold cross-validation is also described below in the Examples.

In some embodiments, a relevance of each candidate gene for classification is determined independently (230). This may be described as scoring each candidate gene independently for relevance in classification. In some embodiments, determining a relevance for each candidate gene for classification includes determining an error of classification, or determining a mean error of classification, for each candidate segment independently. In some embodiments, determining a relevance for each candidate gene for classification includes determining a mean error of classification for each candidate segment independently based on k-fold cross validation using a naïve Bayesian (NB) classifier with the candidate segment as the input attribute. In some embodiments, the training data is divided into multiple subsets, which may be described as k subsets or k folds (e.g., k=10) for cross-validation. For each individual candidate segment of a gene i, a classifier (e.g., a naïve Bayesian (NB) classifier) is constructed on the training of k-1 (e.g., 9) subsets using the CNV data from the individual candidate segment as the input attribute. The performance of the classifier constructed on the training of k-1 subsets is then tested using the other subset that wasn't used for training to determine a classification error for the fold j:

$$classification {error}_{j} = \frac{incorrect preaictions}{total {preaictions}_{j}} ,$$

which may be expressed as a ratio or as a percentage.

The training and testing subsets are then rotated for each of the k folds, and the average or mean of the classification errors across the folds is used to rank the relative relevance of the candidate segment of a gene i. The average or mean classification error across the folds for candidate segment i can be calculated as follows

$$classification {error}_{a v g} = \frac{{{Σ}_{j = 1}^{k} ( \frac{incorrect predictions}{total predictions} )}_{i}}{k} .$$

The procedure is repeated for each candidate segment i. The average or mean classification error for the candidate segments are used to rank the candidate segments from lowest average classification error to the highest average classification error, where the lowest average classification error corresponds to the best assigned rank and the highest relative relevance for the candidate segment.

A first most relevant subset of the candidate segments, referred to herein as a first relevant subset or a current relevant subset, is selected based on the subset of the candidate segments having the best assigned rank with respect to the average or mean classification error for each candidate segment, a GMNB classifier is traited with the selected relevant subset of candidate segments as input attributes, and effectiveness for the trained GMNB classifier is determined (240). In some embodiments, the first relevant subset may include all of the candidate segments sequenced from the target genes. In some embodiments, the first relevant subset may only include a subset of the most relevant candidate segments based on the rank for each segment (e.g., top ranked 95% of candidate segments, top ranked 90% of candidate segments, top ranked 85% of candidate segments, top ranked 80% of candidate segments, top ranked 80% of candidate segments, top ranked 75% of candidate segments, top ranked 70% of candidate segments, top ranked 65% of candidate segments, top ranked 60% of candidate segments, top ranked 65% of candidate segments, top ranked 60% of candidate segments, top ranked 55% of candidate segments, top ranked 50% of candidate segments, top ranked 45% of candidate segments, top ranked 40% of candidate segments, top ranked 35% of candidate segments, top ranked 30% of candidate segments, top ranked 25% of candidate segments, top ranked 20% of candidate segments, top ranked 25% of candidate segments, top ranked 20% of candidate segments, top ranked 15% of candidate segments, or top ranked 10% of candidate segments, top ranked 5% of candidate segments).

CRV data for the first relevant subset of the candidate segments is then employed for training a first Geometric Mean Naïve Bayesian (GMNB) classifier. The effectiveness of the first trained GMNB classifier or current trained GMNB classifier is then measured. In some embodiments, the training of the first GMNB classifier and measurement of the effectiveness of the first trained GMNB classifier is conducted using cross-validation, where test data is divided into subgroups or folds. All but one of the subgroups are used for training the first trained GMNB classifier, and the subgroup not used for training the GMNB classifier is used for testing the GMNB classifier for that fold. This is repeated with each of the subgroups serving as the test subgroups, and the measurement of the effectiveness of the first trained GMNB classifier is averaged across the folds. The Area under the ROC curve (AUC) is used as the measurement of effectiveness, which may also be referred to as the effectiveness score, in some embodiments.

In some embodiments, one or more of the least relevant candidate segments are removed from the first relevant subset to create a modified or second relevant subset, which is now the current relevant subset with the first relevant subset now being referred to as the immediately prior relevant subset (250). CRV data for the modified second relevant subset is then employed for training a second Geometric Mean Naïve Bayesian (GMNB) classifier, which may be referred to as a modified trained GMNB classifier or a current trained GMNB classifier. The effectiveness of the second trained GMNB classifier/modified trained GMNB classifier/current trained GMNB classifier is then measured (260). A cross-fold validation procedure may be used to train the second GMNB classifier/modified GMNB classifier/current GMNB classifier and to measure the effectiveness for each fold, and an average effectiveness across all the folds may be determined for the second trained GMNB classifier/modified trained GMNB classifier/current trained GMNB classifier.

The average measured effectiveness of the second trained GMNB classifier/modified trained GMNB classifier/current trained GMNB classifier across all folds is compared to the that of the first trained GMNB classifier/immediately prior trained GMNB classifier to determine whether the removal of one or more of the least relevant candidate segments in the subset of relevant candidate segments had a positive impact on effectiveness, had a negative impact on effectiveness, or had no statistically significant impact on effectiveness (270). In some embodiments, a paired T-test two sample is performed between the average measured effectiveness of the second trained GMNB classifier/modified trained GMNB classifier/current trained GMNB classifier across all folds and that of the first trained GMNB classifier/immediately prior trained GMNB classifier across all folds to determine whether the removal of one or more of the least relevant candidate segments had a statistically significant impact on effectiveness.

If the effectiveness of the second trained GMNB classifier/modified trained GMNB classifier/current trained GMNB classifier is worse than that of the first trained GMNB classifier/immediately prior trained GMNB classifier, the first relevant subset of candidate segments, which is the immediately prior subset of relevant candidate segments is selected as the plurality of target segments for training the GMNB classifier. Training data for this plurality of target segments is used to generate trained GMNB classifier for use (280). In some embodiments, all of the prior training data for the plurality of target segments is used to generate the trained GMNB classifier for use on new data. In some embodiments, new training data for the plurality of target segments is used to generate the trained GMNB classifier for use on new data.

If the effectiveness of the second trained GMNB classifier/modified trained GMNB classifier/current trained GMNB classifier is better or statistically the same as that of the first trained GMNB classifier/immediately prior trained GMNB classifier, then another one or more of the least relevant candidate segments are removed from the second relevant subset of candidate segments forming a modified subset, which is now the current relevant subset of candidate segments (250), and the current relevant subset is used to train a new current GMNB classifier, an effectiveness score is measured for the new current trained GMNB classifier (260), and the effectiveness score for the new current trained GMNB classifier is compared to the effectiveness score for the immediately prior trained GMNB classifier (270).

This modification of the subset of relevant candidate segments resulting in a new current subset, training of the new current GMNB classifier based on the new current subset, measurement of effectiveness of the new current GMNB classifier, and comparison of effectiveness of the new current trained GMNB classifier to that of the immediately prior trained GMNB classifier may be repeated, as needed, in an iterative process until the effectiveness score for the new current trained GMNB classifier is worse than the effectiveness score for the immediately prior trained GMNB classifier, at which point the immediately prior relevant subset of candidate segments is selected or identified as the plurality of target segments for training the GMNB classifier. In some embodiments, a new trained GMNB classifier may be generated based on all the prior training data for the plurality of target segments to be the trained classifier for use in classifying new samples from subjects. In some embodiments, a new trained GMNB classifier may be generated based on new training data for the plurality of final segments to be the trained classifier for use in classifying new samples from subjects.

One of ordinary skill in the art in view of the present disclosure will appreciate the above-described method for identifying the plurality of target segments may alternatively involve initially selecting a smaller subset of most relevant candidate segments, training a GMNB classifier on the subset of most relevant candidate segments, evaluating an effectiveness of the trained GMNB classifier, and then modifying the subset of most relevant candidate segments by adding one or more of the next most relevant candidate segments to create a new current most relevant subset of candidate segments. The new current most relevant subset of candidate segments would be used to train a new GMNB classifier, and an effectiveness of new current trained GMNB classifier would be determined. If the effectiveness of the new current trained GMNB classifier is statistically better than that of the immediately prior trained GMNB classifier, the subset of the most relevant candidate segments would be modified by adding one or more of the most relevant candidate segments to create a new current most relevant subset and the cycle repeated. If the effectiveness of the new current trained GMNB classifier is the statistically the same as or worse than that of the immediately prior trained GMNB classifier, the immediately prior relevant subset of candidate segments is identified or selected as the plurality of target segments that are used to train the new GMNB classifier for use to evaluate new samples.

One of ordinary skill in the art in view of the present disclosure will appreciate that the method of selecting the plurality of target segments may include a procedure that involves one or more steps of adding one or more most relevant segments to a subset of relevant candidate segments to form a new current relevant subset of candidate segments and one or more steps of removing one or more least relevant segments from a subset of relevant candidate segments to form a new current relevant subset of candidate segments.

Naïve Bayesian Classifier and Geometric Mean Naïve Bayesian (GMNB) Classifier

The naïve Bayesian classifier is a simple but often effective machine learning algorithm. It is based on Bayes' theorem and the assumption that all attributes are conditionally independent.

Let (x₁, x₂, . . . , x_{d}) be the input attribute vector and (C₁, C₂, . . . , Cₖ) be the classes.

According to Bayes Theorem,

$$P ( {C}_{j} \vert {x}_{1} , {x}_{2} , … {x}_{d} ) = \frac{P ( {c}_{j} ) P ( {x}_{1} , {x}_{2} , … {x}_{d} \vert {C}_{j} )}{{Σ}_{i = 1}^{k} P ( {C}_{i} ) P ( {x}_{1} , {x}_{2} , … , {x}_{d} \vert {C}_{i} )} .$$

With the assumption of conditional independence,

P(x₁,x₂, . . . x_{d}|Cⱼ)=P(x₁|Cⱼ)P(x₂|Cⱼ) . . . P(x_{d}|Cⱼ)

The probabilities P(xᵢ|Cⱼ) can be easily estimated from training data. However, when the dimension d is large, the products of the probabilities (likelihood) becomes extremely small, causing underflows. If each probability value has an average of 1/2, the likelihood will have a mean

$$E [ P ( {x}_{1} \vert {C}_{j} ) P ( {x}_{2} \vert {C}_{j} ) … P ( {x}_{d} \vert {C}_{j} ) ] = \frac{1}{{2}^{d}}$$

which approaches 0 quickly when d is large.

One typical method to avoid numerical underflow is to scale all the values using the largest probability product during the computations. However, this method often produces one value that dominates the probability products. As a result, one class will have the predicted probability of 1.0 while all other classes will have a prediction probability of 0.0.

This effect is disadvantageous for most applications because it is an artifact of the naïve Bayesian assumption and usually does not reflect the real probability.

The inventors propose a generalization to the standard naïve Bayesian algorithm to address the underflow problem. Let h(x) be a positive increasing function. Applying the function to the likelihood produces a new probability estimate:

P(x₁,x₂, . . . x_{d}|Cⱼ)=h[P(x₁|Cⱼ)P(x₂|Cⱼ) . . . P(x_{d}|Cⱼ)].

In particular, the inventors propose to use the function

h(x,d)=x1/d,

which increases monotonically with d and prevents underflow for any dimension d. This modified probability estimate employing the positive increasing function h(x, d) is termed the Geometric Mean Naïve Bayesian (GMNB) classifier herein.

Lemma. Let x be a uniform random value over the interval [0, 1]; the expected value of x h(x, d)=x 1/dfor a constant d is

$$\frac{1}{( 1 + 1 / d )} .$$

Proof Because x is uniform, the expected value of X^{1/d} is

$${{\int }_{0}^{1} {x}^{1 / d} dx = \frac{{x}^{1 + 1 / d}}{1 + 1 / d} \vert}_{0}^{1} = \frac{1}{( 1 + 1 / d )} .$$

Theorem. Assume that the probabilities in the likelihood are independent, uniformly distributed random variables. Then, the expected value of the likelihood is

$$E [ {( P ( {x}_{1} \vert {C}_{j} ) P ( {x}_{2} \vert {C}_{j} ) … P ( {x}_{d} \vert {C}_{j} ) )}^{\frac{1}{d}} ] = \frac{1}{{( 1 + 1 / d )}^{d}} .$$

Proof By the previous lemma and the independence of the random variables,

$$\begin{matrix}E [ {( P ( {x}_{1} \vert {C}_{j} ) P ( {x}_{2} \vert {C}_{j} ) … P ( {x}_{d} \vert {C}_{j} ) )}^{\frac{1}{d}} ] = E [ {( P ( {x}_{1} \vert {C}_{j} ) )}^{\frac{1}{d}} ] \\ E [ {( P ( {x}_{2} \vert {C}_{j} ) )}^{\frac{1}{d}} ] \\ … E [ {( P ( {x}_{d} \vert {C}_{j} ) )}^{\frac{1}{d}} ] \\ = \frac{1}{{( 1 + 1 / d )}^{d}}\end{matrix}$$

The limit of the expected value is

$${lim}_{d \to ∞} \frac{1}{{( 1 + 1 / d )}^{d}} = \frac{1}{e} .$$

Therefore, as the dimension increases, the likelihood will never approach 0 uniformly. Applying the function h to the likelihood does not change the relative order of the probability estimates of the classes. However, the probabilities will have more reasonable values than 0 and 1.

The inventors can also show that the function h(x, d)=x^{1/d} is unique under certain conditions.

Lemma. Let ƒ(x) be a positive continuous function of positive real numbers. If ƒ is multiplicative, ƒ(xy)=ƒ(x)ƒ(y), then ƒ(x)=x^{α} for some constant α.

In the case of the functional transform on the likelihood, the assumption of the multiplicative property on the function h is a natural extension of the naïve Bayesian assumption. If it is required that the likelihood approaches a non-zero limit as d approaches infinity, then the function could have the form h(x, d)=x^{c/d} for a constant c.

Theorem. If h is multiplicative and

$${lim}_{d \to ∞} E [ ( P ( {x}_{1} \vert {C}_{j} ) P ( {x}_{2} \vert {C}_{j} ) ) … P ( {x}_{d} \vert {C}_{j} ) ] = L > 0 ,$$

$$then h ( x , d ) = {x}^{a ( d )} , where a ( d ) = d ( \frac{1}{d} ) + O ( \frac{1}{{d}^{2}} ) , c > 0 .$$

Proof The previous lemma shows that

h(x,d)=x^{a(d)}

Similar to the previous proof, the expectation is

$$\begin{matrix}E [ h ( P ( {x}_{1} \vert {C}_{j} ) P ( {x}_{2} \vert {C}_{j} ) … P ( {x}_{d} \vert {C}_{j} ) ) ] = E [ {( P ( {x}_{1} \vert {C}_{j} ) P ( {x}_{2} \vert {C}_{j} ) … P ( {x}_{d} \vert {C}_{j} ) )}^{a ( d )} ] \\ = E [ {( P ( {x}_{1} \vert {C}_{j} ) )}^{a ( d )} ] , \\ E [ {( P ( {x}_{2} \vert {C}_{j} ) )}^{a ( d )} ] … E [ {( P ( {x}_{d} \vert {C}_{j} ) )}^{a ( d )} ] \\ = \frac{1}{{( 1 + a ( d ) )}^{d}}\end{matrix} .$$

By the assumption, we have

$${lim}_{d \to ∞} \frac{1}{{( 1 + a ( d ) )}^{d}} = L > 0.$$

$$Letting t = 1 / d and f ( t ) = a ( 1 / t ) = a ( d ) ,$$

$$then {lim}_{d \to ∞} \frac{1}{{( 1 + a ( d ) )}^{d}} = {lim}_{t \to 0 +} \frac{1}{{( 1 + f ( t ) )}^{\frac{1}{t}}} = {lim}_{t \to 0 +} {e}^{\frac{- ln ( 1 + f ( t ) )}{t}} .$$

$$Furthermore , f ( 0 + ) = 0$$

$$and {lim}_{t \to 0} {e}^{\frac{- ln ( 1 + f ( t ) )}{t}} = {lim}_{t \to 0} {e}^{\frac{- {f}^{′} ( t )}{1 + f ( t )}} = {lim}_{t \to 0} {e}^{- {f}^{′} ( t )} = {e}^{- c} = L .$$

Therefore,

$$f ( t ) = ct + O ( {t}^{2} ) ,$$

$$a ( d ) = c ( \frac{1}{d} ) + O ( \frac{1}{{d}^{2}} ) , c > 0.$$

When the dimension d is high, the independence assumption of the naïve Bayesian classifier is unlikely to be true in most applications. Consequently, the probability estimates are unrealistic. The proposed extension can solve this problem.

As an example illustrating the accuracy of the proposed Geometric Mean Naïve Bayesian (GMNB) classifier as compared to the original Naïve Bayesian (NB) classifier, consider a two-class problem with d-dimensional Gaussian distributions, with means of (1, 1, . . . , 1 and (−1,−1, . . . , −1) and the same covariance matrix

$$[ \begin{matrix}1 & r & … & r \\ r & 1 & … & r \\ ⋮ & ⋮ & ⋱ & ⋮ \\ r & r & … & 1\end{matrix} ] = ( 1 - r ) I + r ;$$

the inverse matrix is

$$\frac{1}{1 - r} ( I - \frac{r}{1 - r + r d} J ) .$$

Consider the probability estimations for the point (t, t, . . . , t). The true probability for class 1 is

$$\frac{{e}^{- 0.5 {d ( t - 1 )}^{2} ( 1 - \frac{r d}{1 - r + r d} )}}{{e}^{- 0.5 {d ( t - 1 )}^{2} ( 1 - \frac{r d}{1 - r + r d} )} + {e}^{- 0.5 {d ( t + 1 )}^{2} ( 1 - \frac{r d}{1 - r + r d} )}} .$$

For the original NB classifier,

$$\frac{{e}^{- 0.5 {d ( t - 1 )}^{2}}}{{e}^{- 0.5 {d ( t - 1 )}^{2}} + {e}^{- 0.5 {d ( t + 1 )}^{2}}} ,$$

and for the proposed GMNB classifier,

$$\frac{{e}^{- 0.5 {( t - 1 )}^{2}}}{{e}^{- 0.5 {( t - 1 )}^{2}} + {e}^{- 0.5 {( t + 1 )}^{2}}} .$$

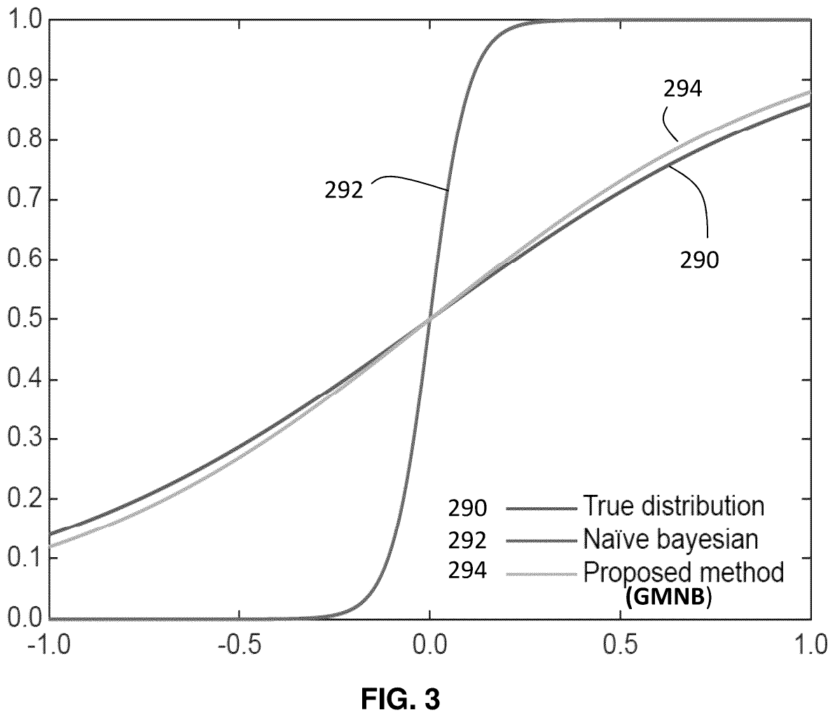

FIG. 3 shows the three probability estimates for d=10 and r=0.5. The NB classifier probability estimates 292 change steeply around the boundary owing to the independence assumption. In contrast, the proposed method with the GMNB classifier produces probability estimates 294 that closely approximate the true probability distribution 290.

BRCA1/2 DNA Repair Pathway

BReast CAncer genes, BRCA1 and BRCA2, are integral to the Fanconi anemia (FA)/BRCA pathway to regulate cellular responses to DNA damage, e.g., DNA interstrand crosslinks, and DNA double strand breaks. The FA/BRCA family of proteins consists of at least 22 FANC genes, description of which is presented in Table 1 below [adapted from Garcia-de-Teresa B, Rodriguez A, Frias S. Chromosome Instability in Fanconi Anemia: From Breaks to Phenotypic Consequences. Genes (Basel). 2020; 11(12):1528. Published 2020 Dec 21].

TABLE 1
Fanconi anemia (FA) and FA-like genes involved in FA/BRCA pathway

| | Cytogenetic | |
|---|---|---|
| FANC Gene/Alias | Location | Function of the FANC Protein |
| FANCA | 16q24.3 | FA core complex |
| FANCB | Xp22.2 | FA core complex |
| FANCC | 9q22.32 | FA core complex |
| FANCD1/BRCA2 | 13q13.1 | Homologous recombination. Enable RAD51 to displace |
| | | RPA from ssDNA. |
| FANCD2 | 3p25.3 | Monoubiquitinated ID complex recruits the downstream |
| | | repair proteins and facilitates repair of DNA ICLs |
| FANCE | 6p21.31 | FA core complex; bridge between the FA core complex |
| | | and FANCD2 |
| FANCF | 11p14.3 | FA core complex |
| FANCG/XRCC9 | 9p13.3 | FA core complex |
| FANCI | 15q26.1 | Monoubiquitinated ID complex recruits the downstream |
| | | repair proteins and facilitates repair of DNA ICLs |
| | | (intrastrand crosslink) |
| FANCJ/BRIP1 | 17q23.2 | FA core complex |
| FANCL | 2p16.1 | E3 ubiquitin-protein ligase, monoubiquitination of |
| | | FANCD2 |
| FANCM | 14q21.2 | FA core complex. Acts by sensing stalled fork by ICLs |
| | | and recruiting the core complex proteins to the site of ICL |
| FANCN/PALB2 | 16q12.2 | Homologous recombination |
| FANCO/RAD51 | 17q22 | Resolution of D-loop structures through Holliday |
| | | Junction Intermediates and Homologous DNA Pairing |
| | | and Strand Exchange. |
| FANCP/SLX4 | 16p13.3 | Cooperate with FANCQ-XPF to generate |
| | | endonucleolytic incisions to unhook the ICL |
| FANCQ/XPF | 16p13.12 | DNA endonuclease, involved in homologous |
| | | recombination; responsible for 50 incision to remove |
| | | ICLs |
| FANCR/RAD51 | 15q15.1 | Interact with the ssDNA-binding protein RPA, RAD52 |
| | | homologous pairing and strand transfer of DNA |
| FANCS/BRCA1 | 17q21.31 | Homologous recombination |
| FANCT/UBE2T | 1q32.1 | E2 ubiquitin-conjugating enzyme, associates with FA |
| | | core complex, catalyzes monoubiquitination of FANCD2 |
| | | in association with FANCL |
| FANCU/XRCC2 | 7q36.1 | Homologous recombination |
| FANCV/REV7 | 1p36.22 | Translesion DNA synthesis |
| FANCW/RFWD3 | 16q23.1 | RING-Type E3 Ubiquitin Transferase |

DNA double strand (dsDNA) breaks can occur because of external DNA damaging agents (e.g., crosslinking agents) or endogenously induced damage (e.g., stalled replication fork resulting double strand break) and repair of dsDNA breaks are detrimental to cell survival. Two pathways, homologous recombination (HR) and nonhomologous end joining (NHEJ), are used to repair dsDNA breaks. HR utilizes homologous regions in the intact chromosome or sister chromatid as the template to repair the broken strand resulting in error-free repair.

Homologous recombination is initiated by invasion of 3′ single strand DNA (ssDNA) tail formation by resection of the dsDNA break, into the homologous DNA template. Holliday junctions are formed after DNA synthesis and second strand invasion. Resolution of Holliday junctions result in crossover or non-cross-over products.

During S/G2 phase, dsDNA breaks signal the FA/BRCA pathway by initiating FA core complex formation (FANCA, B, C, E, F, G, L, M, and N) and formation of the FANCI/FANCD2 complex, which localize with BRCA1, BRCA2 and other DNA repair enzymes at DNA repair foci. BRCA2 (also known as FAND1) is involved in homologous recombination by enabling RAD51 (also known as FANCR, which is a recombinase) to displace RPA (replication protein A) from ssDNA during strand invasion. BRCA1 (also known as FANCS) is also involved in homologous recombination.

Homologous recombination deficiency (HRD) is a phenotype that is characterized by the inability of a cell to effectively repair DNA double-strand breaks using the homologous recombination pathway. For example, a mutated or deleted BRCA1 or BRCA2 gene decreases DSB repair efficiency in cells and makes the cells sensitive to DSB-inducing agents for cancer treatment. The level of HRD is dependent on which DNA repair gene(s) are mutated or deleted. For example, BRCA1/2 are critical to the pathway and mutations and/or deletions in either gene would result in greater sensitivity to DSB-inducing agents. Alternatively, mutations and/or deletions in genes which are redundant or less important to the homologous recombination pathway would result in lower sensitivity to DSB-inducing agents.

DNA double strand break (DSB) deficiency is a phenotype that is characterized by the inability of a cell to effectively repair DNA double-strand breaks using homologous recombination (HR) or non-homologous end joining (NHEJ). As in HRD, mutations and/or deletions in any HR and/or NHEJ genes can result in a level of DSB deficiency. Examples of NHEJ genes include, but are not limited to, KU70/80, DNA-PKcs, Mre11/Rad50/Nbs1, Artemis and XLF/XRCC4. A greater level of DSB deficiency in a cell makes the cell more sensitive to DSB-inducing agents for cancer treatment.

### Computing Systems, Networks and Devices for Implementing Some Embodiments

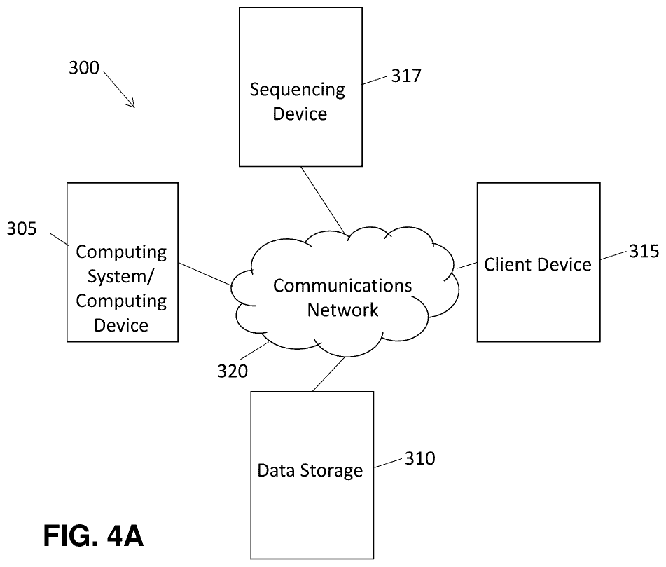

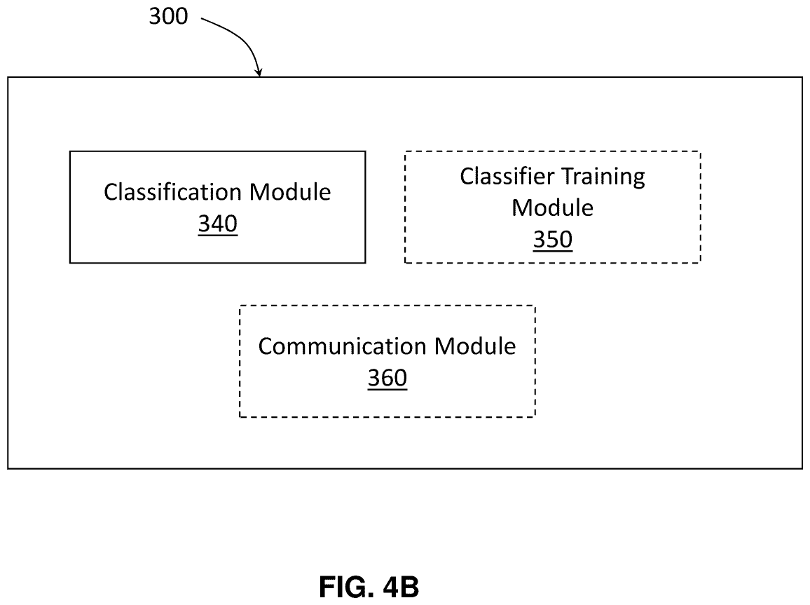

FIG. 4A schematically depicts a network 300, alternately described as a networked computing system, for implementing some aspects in accordance with some embodiments.

Network 300 may include at least one computing system 305, at least one client device 315, and data storage 310 that may be in the form of one or more databases. In some embodiments, network 300 may also include a sequencing device 317. In some embodiments, computing system 305, client device 315, sequencing device 317, and/or data storage 310 may be connected to network 320. However, in other embodiments, two or more of computing system 305, client device 315, sequencing device 317, and/or data storage 310 may be connected directly with each other, without network 320. While one computing system 305, one client device 315, one sequencing device 317, and one data storage 310 are shown in FIG. 4A, it should be appreciated that any number of computing systems, client devices, sequencing devices, and data storages could be used.

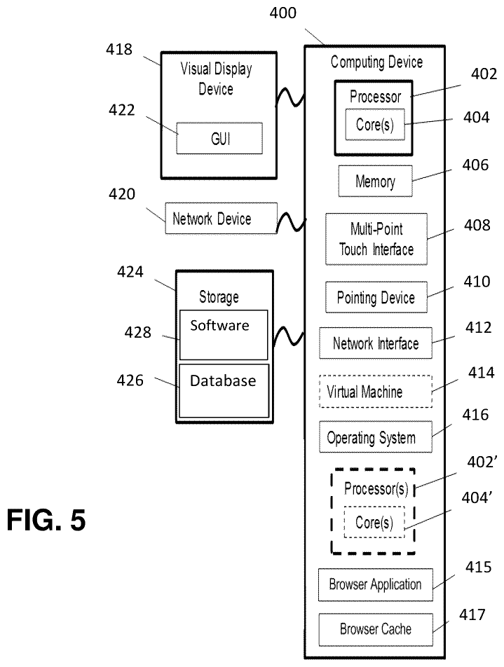

Computing system 305 may include one or more computing devices configured to perform one or more operations consistent with disclosed embodiments. Computing system 305 is further described in connection with FIG. 5. In some embodiments, computing system 305 may perform at least some aspects or steps of the described methods. In some embodiments, computing system 305, sequencing device 317, and/or client device 315 may perform at least some aspects or steps of the described methods in some embodiments. For example, in some embodiments, sequencing device 317 is employed for sequencing one or more target genes or fragments thereof (e.g., target sequences or candidate sequences) from a biological sample and/or a reference sample. For example, the sequencing device may be employed for sequencing the adaptor-ligated DNA fragments of one or more target genes or fragments thereof (e.g., target sequences or candidate sequences) in a biological sample and/or in a reference sample. In some embodiments, sequencing device 317 and computing system 305 employed for sequencing one or more target genes or fragments thereof (e.g., target sequences or candidate sequences) from a biological sample and/or a reference sample. For example sequencing device 317 and computing system 305 employed are employed for sequencing the adaptor-ligated DNA fragments of one or more target genes or fragments thereof (e.g., target sequences or candidate sequences) in a biological sample and/or in a reference sample.

In some embodiments, computing system 305 is employed for determining a copy number of one or more target genes or fragments (e.g., target sequences or candidate sequences) of a biological sample and/or of a reference sample. In some embodiments, computing system 305 and/or client device 315 are employed for determining a copy number of one or more target genes or fragments (e.g., target sequences or candidate sequences) of a sample and/or of a reference sample from the sequences of the adaptor-ligated DNA fragments for the biological sample and/or for the reference sample.

In some embodiments, computing system 305 classifies the sample by applying a trained classifier using the copy number variation for the plurality of target segments as attributes for the trained classifier. In some embodiments, computing system 305 and/or client device 315 classify the sample by applying a trained classifier using the copy number variation for the plurality of target segments as attributes for the trained classifier.

In some embodiments, computing system 305 identifies the subject from which the sample was obtained as a candidate for treatment with a double strand break-inducing agent based on the classification of the sample. In some embodiments, computing system 305 and/or client device 315 identify the subject from which the sample was obtained as a candidate for treatment with a double strand break-inducing agent based on the classification of the sample.

In some embodiments, computing system 305 and/or client device 315 display (e.g., in a graphical user interface) an identification of the subject as a candidate for treatment with a double strand break-inducing agent. In some embodiments, computing system 305 and/or client device 315 display (e.g., in a graphical user interface) a recommendation for treatment of the subject with a double strand break-inducing agent. In some embodiments, computing system 305 and/or client device 315, transmit an identification of or information regarding an identification of the subject as a candidate for treatment with a double strand break-inducing agent. In some embodiments, computing system 305 and/or client device 315 transmit a recommendation for treatment of the subject with a double strand break-inducing agent.

In some embodiments, computing system 305, client device 315, data storage 310, or a combination of the aforementioned store an identification of or information regarding an identification of the subject as a candidate for treatment with a double strand break-inducing agent. In some embodiments, computing system 305, client device 315, data storage 310, or a combination of the aforementioned store a recommendation for treatment of the subject with a double strand break-inducing agent.

Data storage 305 may include one or more computing devices configured with appropriate software to perform operations consistent with storing and providing data. Data storage 305 may include, for example, Oracle™ databases, Sybase™ databases, or other relational databases or non-relational databases, such as Hadoop™ sequence files, HBase™, or Cassandra™. Data storage 305 may include computing components (e.g., database management system, database server, etc.) configured to receive and process requests for data stored in memory devices of data storage 305 and to provide data from data storage 305. In some embodiments, data storage 305 may be configured to store the dataset including cell-free DNA sequencing data used by computing system 305. In some embodiments, data storage 305 may be configured to store CNVkit software used by computing system 305.

While data storage 305 is shown separately, in some embodiments, data storage 305 may be included in or otherwise related to computing system 305 and/or client device 315.

Client device 315 may include a desktop computer, a laptop, a server, a mobile device (e.g., tablet, smart phone, etc.), a wearable computing device, or other type of computing device. Client device 315 may include one or more processors configured to execute software instructions stored in memory, such as memory included in client device 315. In some embodiments, client device 315 may include software that when executed by a processor performs known Internet-related communication and content display processes. For instance, client device 315 may execute browser software that generates and displays interfaces including content on a display device included in, or connected to, client device 315. Client device 315 may execute applications that allows client device 315 to communicate with components over network 170 and generate and display content in interfaces via display devices included in client device 315. For example, client device 315 may display results produced by computing system 305, such as qualified subjects for chemotherapy or immunotherapy. Computing system 305 may communicate the results to the client device 315.

Computing system 305, client device 315, and database 315 are shown as a different components. However, computing system 305, client device 315, and/or database 315 may be implemented in the same computing system or device. For example, computing system 305, client device 315, and/or database 315 may be embodied in a single computing device.

Network 320 may be any type of network configured to provide communications between components of network 320. For example, network 320 may be any type of network (including infrastructure) that provides communications, exchanges information, and/or facilitates the exchange of information, such as the Internet, a Local Area Network, near field communication (NFC), optical code scanner, or other suitable connection(s) that enables the sending and receiving of information between the components of network 320. In other embodiments, one or more components of network 320 may communicate directly through a dedicated communication link(s).

FIG. 4B is a block diagram showing a system 300 implemented, at least in part, in one or more module according to an example embodiment. In some embodiments, the one or more modules include a classification module 340. In some embodiments, the one or more modules also include a classifier training module 350. In some embodiments, the one or more modules also include a communication module 360. In some embodiments, the modules 340, 350 and 360 are included in the same system. In an example embodiment, the modules 340, 350 and 360 are included in computing system 305. In another example embodiment, at least one of modules 340, 350 and 360 is implemented, at least in part, in computing system 305 and at least one other of modules 340, 350 and 360 is implemented, at least in part, in the client device 315 and/or the sequencing device 317.

In some embodiments, the modules 340, 350 and 360 may be implemented, at least in part, in any of computing system 305, client device 315, and sequencing device 317. The modules 340, 350 and 360 may include one or more software components, programs, applications, apps or other units of code base or instructions configured to be executed by one or more processors included in devices 305 and 315.

Although modules 340, 350, and 360 are shown as distinct modules in FIG. 4B, it should be understood that modules 340, 350, and 360 may be implemented as fewer or more modules than illustrated. It should be understood that any of modules 340, 350, and 360 may communicate with one or more external components such as databases, servers, database server, or other devices.

In some embodiments, the classification module 340 is a software-implemented module, or a module implemented in part in software and in part in hardware, and is configured to classify as sample. In some embodiments, the classification module 340 is further configured to identify a subject as a candidate for treatment.

In some embodiments, the classifier training module 350 is a software-implemented module, or a module implemented in part in software and in part in hardware, and is configured to generate the trained classifier. In some embodiments, classifier training module 350 is also configured to determine the plurality of target segments whose copy number variation is used for classification.

In some embodiments, the communication module 360 is a software-implemented module, or a module implemented in part in software and in part in hardware and configured to send an electronic communication including an identification of a subject as a candidate for treatment with a double strand break-inducing agent, configured to send an electronic communication including a recommendation of treatment with a double strand break-inducing agent chemotherapy or immunotherapy for a subject, or both.

Certain embodiments are described herein as including logic or a number of components, modules, or mechanisms. Modules may constitute either software modules (e.g., code embodied on a machine-readable medium or in a transmission signal) or hardware modules. A hardware module is a tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may include dedicated circuitry or logic that is permanently configured (e.g., as a special-purpose processor, such as a field programmable gate array (FPGA), an application-specific integrated circuit (ASIC), or a Graphics Processing Unit (GPU)) to perform certain operations. A hardware module may also include programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired) or temporarily configured (e.g., programmed) to operate in a certain manner and/or to perform certain operations described herein. Considering embodiments in which hardware modules are temporarily configured (e.g., programmed), each of the hardware modules need not be configured or instantiated at any one instance in time. For example, where the hardware modules include a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connect the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, include processor-implemented modules.

Similarly, the methods described herein may be at least partially processor-implemented. For example, at least some of the operations of a method may be performed by one or processors or processor-implemented modules. The performance of certain of the operations may be distributed among the one or more processors, not only residing within a single machine, but deployed across a number of machines. In some example embodiments, the processor or processors may be located in a single location (e.g., within a home environment, an office environment or as a server farm), while in other embodiments the processors may be distributed across a number of locations.

The one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., APIs).

Example embodiments may be implemented in digital electronic circuitry, or in computer hardware, firmware, software, or in combinations of them. Example embodiments may be implemented using a computer program product, for example, a computer program tangibly embodied in an information carrier, for example, in a machine-readable medium for execution by, or to control the operation of, data processing apparatus, for example, a programmable processor, a computer, or multiple computers.

For the purposes of this disclosure, a non-transitory computer readable medium stores computer programs and/or data in machine readable form. By way of example, and not limitation, a computer readable medium can include computer storage media and communication media. Computer storage media includes volatile and non-volatile, removable and non-removable media implemented in any method or technology for storage of information such as computer-readable instructions, data structures, program modules, and specific applications.

A computer program can be written in any form of programming language, including compiled or interpreted languages, and it can be deployed in any form, including as a stand-alone program or as a module, subroutine, or other unit suitable for use in a computing environment. A computer program can be deployed to be executed on one computer or on multiple computers at one site or distributed across multiple sites and interconnected by a communication network.

In example embodiments, operations may be performed by one or more programmable processors executing a computer program to perform functions by operating on input data and generating output. Method operations can also be performed by, and apparatus of example embodiments may be implemented as, special purpose logic circuitry (e.g., a FPGA or an ASIC).

The computing system can include clients and servers. A client and server are generally remote from each other and typically interact through a communication network.

The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., machine) and software architectures that may be deployed, in various example embodiments.

FIG. 5 schematically depicts a computing system 400 for implementing some aspects in accordance with some embodiments. In some embodiments, computing device 400 may be computing system 305 shown in FIG. 4. In some embodiments, computing device 400 may be client device 315 shown in FIG. 4. Computing device 400 includes one or more non-transitory computer-readable media for storing one or more computer-executable instructions or software for implementing exemplary embodiments. The non-transitory computer-readable media can include, but are not limited to, one or more types of hardware memory, non-transitory tangible media (for example, one or more magnetic storage disks, one or more optical disks, one or more USB flash drives), and the like. For example, memory 406 included in the computing device 400 can store computer-readable and computer-executable instructions or software for implementing exemplary embodiments. Computing device 400 also includes processor 402 and associated core 404, and optionally, one or more additional processor(s) 402′ and associated core(s) 404′ (for example, in the case of computer systems having multiple processors/cores), for executing computer-readable and computer-executable instructions or software stored in the memory 406 and other programs for controlling system hardware. Processor 402 and processor(s) 402′ can each be a single core processor or multiple core (404 and 404′) processor.

Virtualization can be employed in computing device 400 so that infrastructure and resources in the computing device can be shared dynamically. A virtual machine 414 can be provided to handle a process running on multiple processors so that the process appears to be using only one computing resource rather than multiple computing resources. Multiple virtual machines can also be used with one processor.

Memory 406 can include a computer system memory or random-access memory, such as DRAM, SRAM, EDO RAM, and the like. Memory 406 can include other types of memory as well, or combinations thereof. An individual can interact with the computing device 400 through a visual display device/graphical user interface (GUI) 418, such as a touch screen display or computer monitor, which can display one or more user interfaces 422 for displaying data to the individual. The visual display device 418 can also display other aspects, elements and/or information or data associated with exemplary embodiments. The computing device 400 can include other input devices and I/O devices for receiving input from an individual, for example, a keyboard, a scanner, or another suitable multi-point touch interface 408, a pointing device 410 (e.g., a pen, stylus, mouse, or trackpad). The keyboard 408 and the pointing device 410 can be coupled to the visual display device 418. The computing device 400 can include other suitable conventional I/O peripherals.

The computing device 400 can also include one or more storage devices 424, such as a hard-drive, CD-ROM, or other computer readable media, for storing data and computer-readable instructions and/or software that implements exemplary embodiments of the system as described herein, or portions thereof. Exemplary storage device 424 can also store one or more databases for storing suitable information required to implement exemplary embodiments. The databases can be updated by an individual or automatically at a suitable time to add, delete or update data in the databases. Exemplary storage device 424 can store datasets 426, software 428, and other data/information used to implement exemplary embodiments of the systems and methods described herein. In some embodiments, the storage includes instructions for a sequencing a copy number variation module and for a classification module.

The computing device 400 can include a network interface 412 configured to interface via one or more network devices 420 with one or more networks, for example, Local Area Network (LAN), Wide Area Network (WAN) or the Internet through a variety of connections including, but not limited to, standard telephone lines, LAN or WAN links (for example, 802.11, T1, T3,56kb, X.25), broadband connections (for example, ISDN, Frame Relay, ATM), wireless connections, processing device area network (CAN), or some combination of any or all of the above. The network interface 412 can include a built-in network adapter, network interface card, PCMCIA network card, card bus network adapter, wireless network adapter, USB network adapter, modem or another device suitable for interfacing the computing device 400 to a type of network capable of communication and performing the operations described herein. Moreover, the computing device 400 can be a computer system, such as a workstation, desktop computer, server, laptop, handheld computer, tablet computer (e.g., the iPad® tablet computer), mobile computing or communication device (e.g., the iPhone® communication device), or other form of computing or telecommunications device that is capable of communication and that has sufficient processor power and memory capacity to perform the operations described herein.

The computing device 400 can run an operating system 416, such as versions of the Microsoft® Windows® operating systems, the different releases of the Unix and Linux operating systems, a version of the MacOS® for Macintosh computers, an embedded operating system, a real-time operating system, an open source operating system, a proprietary operating system, an operating systems for mobile computing devices, or another operating system capable of running on the computing device and performing the operations described herein. In exemplary embodiments, the operating system 416 can be run in native mode or emulated mode. In an exemplary embodiment, the operating system 416 can be run on one or more cloud machine instances.

### Example—Prediction of Brca1/2 and Hrd Using Next Generation Sequencing and CNV

Copy number variation (CNV) generated from routine targeted Next Generation Sequencing (NGS) along with machine learning was used for the prediction of the presence of HRD (e.g., classification of tumors as having a BRCA1/2 mutation or genomic structural abnormalities similar to a BRCA1/2 mutation that causes HRD) in various types of tumors.

The inventors reasoned that the key for predicting the presence or absence of HRD was to compare genomic abnormalities of tumors with those BRCA1/2 mutation-positive tumors.

Copy number variation (CNV) abnormalities detected in BRCA1/2 mutation-positive cases were employed along with a machine learning method to build a model for predicting HRD and a BRCA1/2 mutation. The model demonstrated very high sensitivity in predicting cases with BRCA1/2 mutations and in predicting cases with similar abnormalities.

Methods

The CNV from NGS of 434 targeted genes was analyzed using CNVkit software to calculate the log 2 of CNV changes. The log 2 values of various sequencing reads (bins) were used in a machine learning algorithm to train the system on predicting tumors with BRCA1/2 mutations and tumors with structural or biological abnormalities similar to those detected in BRCA1/2 mutations.

Patient Samples

Formalin-fixed, paraffin-embedded (FFPE) cancer samples were sequenced using a targeted NGS panel of 434 genes. The panel of 434 genes was selected for relevance to cancer in general and includes tumor repair genes and inherited double strand repair genes.

The targeted 434 genes are listed in Table 2 below. The cancer samples included 31 samples from patients with confirmed BRCA1/BRCA2 mutations, 84 cancer samples with no evidence of mutations in BRCA1/2 or any DSB repair genes, 114 cancer samples with mutations in one of the genes involved in DSB repair, and 213 additional samples without mutations in any of the DSB genes (DSB-null). The tumor tissue was macrodissected from slides and only samples with tumor percentage at 30% or greater were included. The tumor sites included breast, ovary, endometrial, lung, colorectal, pancreas, and others. Study data was collected as approved by the IRB (WCG IRB #1-1476184-1).

TABLE 2
Target Genes Sequenced

| | | | | | | | | |
|---|---|---|---|---|---|---|---|---|
| ABCB7 | BTK | DDX41 | FGFR2 | IGF1R | MDM4 | PIK3C2B | RNF168 | TCF3 |
| ABL1 | C11orf30 | DICER1 | FGFR3 | IGF2 | MED12 | PIK3CA | RNF43 | TCIRG1 |
| ABL2 | C15ORF41 | DKC1 | FGFR4 | IKBKE | MEF2B | PIK3CB | ROS1 | TERC |
| ACD | CALR | DNM2 | FH | IKZF1 | MEFV | PIK3CG | RPTOR | TERF1 |
| ACVR1B | CARD11 | DNMT3A | FLCN | IKZF3 | MEN1 | PIK3R1 | RTEL1 | TERF2 |
| ADA | CBFB | DOT1L | FLI1 | IL2RG | MET | PIK3R2 | RUNX1 | TERF2IP |
| AK2 | CBL | EED | FLT1 | IL7R | MITF | PIM1 | RUNX1T1 | TERT |
| AKT1 | CBLB | EGFR | FLT3 | INHBA | MLH1 | PLCG1 | SAMD9L | TET2 |
| AKT2 | CBLC | EGLN1 | FLT4 | INPP4B | MPL | PLCG2 | SBDS | TGFBR2 |
| AKT3 | CCND1 | ELANE | FOXL2 | IRF2 | MRE11A | PMS1 | SBF2 | TNFAIP3 |
| ALK | CCND2 | EP300 | FOXP1 | IRF4 | MSH2 | PMS2 | SDHA | TNFRSF14 |
| AMER1 | CCND3 | EPAS1 | FRS2 | IRS2 | MSH6 | POLD1 | SDHB | TNFRSF1A |
| ANKRD26 | CCNE1 | EPCAM | FUBP1 | JAGN1 | MTOR | POLE | SDHC | TOP1 |
| APC | CD274 | EPHA3 | G6PC3 | JAK1 | MUTYH | POT1 | SDHD | TOP2A |
| AR | CD79A | EPHA5 | GABRA6 | JAK2 | MVK | PPM1D | SEC23B | TP53 |
| ARAF | CD79B | EPHA7 | GALNT12 | JAK3 | MYC | PPP2R1A | SETBP1 | TRAF3 |
| ARFRP1 | CDAN1 | EPHB1 | GATA1 | JUN | MYCL | PRDM1 | SETD2 | TSC1 |
| ARID1A | CDC73 | ERBB2 | GATA2 | KAT6A | MYCN | PREX2 | SF3B1 | TSC2 |
| ARID1B | CDH1 | ERBB3 | GATA3 | KDM5A | MYD88 | PRKAR1A | SLIT2 | TSHR |
| ARID2 | CDK12 | ERBB4 | GATA4 | KDM5C | NBN | PRKCI | SLX4 | U2AF1 |
| ASXL1 | CDK4 | ERCC4 | GATA6 | KDM6A | NF1 | PRKDC | SMAD2 | U2AF2 |
| ATG2B | CDK6 | ERG | GEN1 | KDR | NF2 | PRSS1 | SMAD3 | VEGFA |
| ATM | CDK8 | ERRFI1 | GFI1 | KEAP1 | NFE2L2 | PRSS8 | SMAD4 | VHL |
| ATR | CDKN1A | ESR1 | GFI1B | KEL | NFKBIA | PSTPIP1 | SMAD9 | WAS |
| ATRX | CDKN1B | ETV6 | GID4 | KIF23 | NHP2 | PTCH1 | SMARCA4 | WHSC1 |
| AURKA | CDKN2A | EXO1 | GLI1 | KIT | NKX2-1 | PTEN | SMARCB1 | WISP3 |
| AURKB | CDKN2B | EZH2 | GLI2 | KLF1 | NLRP3 | PTPN11 | SMC1A | WT1 |
| AURKC | CDKN2C | FAM175A | GNA11 | KLHL6 | NME1 | QKI | SMC3 | XPO1 |
| AXIN1 | CEBPA | FAM46C | GNA13 | KLLN | NOP10 | RAB27A | SMO | XRCC2 |
| AXIN2 | CHD2 | FANCA | GNAQ | KMT2A | NOTCH1 | RAC1 | SNCAIP | XRCC3 |
| AXL | CHD4 | FANCB | GNAS | KMT2B | NOTCH2 | RAD21 | SOCS1 | ZBTB2 |
| B2M | CHEK1 | FANCC | GPR124 | KMT2C | NOTCH3 | RAD50 | SOX10 | ZNF217 |
| BAP1 | CHEK2 | FANCD2 | GREM1 | KMT2D | NPM1 | RAD51 | SOX2 | ZNF703 |
| BARD1 | CIC | FANCE | GRIN2A | KRAS | NRAS | RAD51B | SOX9 | ZRSR2 |
| BCL2 | CREBBP | FANCF | GRM3 | LIG4 | NROB1 | RAD51C | SPEN | |
| BCL2L1 | CRKL | FANCG | GSK3B | LMO1 | NSD1 | RAD51D | SPOP | |
| BCL2L2 | CRLF2 | FANCI | GSKIP | LPIN2 | NTRK1 | RAD54L | SPTA1 | |
| BCL6 | CSF1R | FANCL | H3F3A | LRP1B | NTRK2 | RAF1 | SRC | |
| BCOR | CSF3R | FANCM | HAX1 | LYN | NTRK3 | RANBP2 | SRSF2 | |
| BCORL1 | CTC1 | FAS | HGF | LYST | NUP93 | RARA | STAG2 | |
| BCR | CTCF | FAT1 | HIST1H3B | LZTR1 | PAK3 | RB1 | STAT3 | |
| BIRC3 | CTNNA1 | FBXW7 | HNF1A | MAGI2 | PALB2 | RBBP6 | STAT4 | |
| BLM | CTNNB1 | FGF10 | HOXA11 | MAP2K1 | PARK2 | RBM10 | STAT6 | |
| BMPR1A | CUL3 | FGF14 | HOXB13 | MAP2K2 | PAX5 | REEP5 | STK11 | |
| BRAF | CUX1 | FGF19 | HRAS | MAP2K4 | PBRM1 | RBM8A | SUFU | |
| BRCA1 | CXCR4 | FGF23 | HSD3B1 | MAP3K1 | PDCD1LG2 | RET | SUZ12 | |
| BRCA2 | CYLD | FGF3 | HSP90AA1 | MAP3K14 | PDGFRA | RHEB | SYK | |
| BRD4 | DAXX | FGF4 | ID3 | MAPK1 | PDGFRB | RHOA | TAF1 | |
| BRIP1 | DDR2 | FGF6 | IDH1 | MCL1 | PDK1 | RICTOR | TAL1 | |
| BTG1 | DDX11 | FGFR1 | IDH2 | MDM2 | PHF6 | RIT1 | TBX3 | |

Next Generation Sequencing

DNA from FFPE was extracted using FormaPure and KingFisher Flex. The extracted DNA from FFPE was sequenced using 100 ng of DNA. A Library for the targeted 434 gene sequencing was based on Single Primer Extension (SPE) chemistry. The DNA sequencing included all coding exons of the 434 genes. For each exon, approximately 50 intronic nucleotides were also sequenced. Genomic DNA samples were end repaired and A-tailed, then unique molecule identifiers (UMIs) and sample index were added. Target enrichment was performed post-UMI assignment to ensure that DNA molecules containing targeted genes were sufficiently enriched in the sequenced library. For enrichment, ligated DNA molecules were subjected to several cycles of targeted PCR using one region-specific primer and one universal primer complementary to the adapter (meaning a synthetic oligonucleotide with known sequence attached to both or either end of DNA fragments for amplification and enrichment of the targeted genes. These synthetic oligonucleotides also incorporate a barcode for multiplexing different samples). A universal PCR was ultimately carried out to amplify the library and add platform-specific adapter sequences and additional sample indices.

The sequencing was conducted using the Illumina NovaSeq 6000 or NextSeq 550 instruments.

Copy Number Variation Evaluation

The CNVkit software was implemented to evaluate CNV in the analyzed samples. Briefly, the software takes advantage of both on- and off-target sequencing reads, compares binned read depths in on- and off-target regions to pooled normal reference, and estimates the copy number at various resolutions. Features of the CNVkit are described above. Additionally, a description of the CVkit software appears in the article “CNVkit: Genome-wide copy number detection and visualization from targeted sequencing” by Talevich et al., which is incorporated by reference herein in its entirety. [Talevich, E., Sham, A. H., Botton, T., & Bastian, B. C. (2014). CNVkit: Genome-wide copy number detection and visualization from targeted sequencing. PLOS Computational Biology 12(4):e1004873]

Using Machine Learning for Classifying Samples

The log 2 of the normalized data of various segments (bins) of the 434 sequenced genes generated by CNVkit (total 26,940 segments) was used in the machine learning approach for predicting the presence or absence of BRCA1/BRCA2 (BRCA1/2) mutations, or for classifying the sample as having a BRCA1/2 mutation or not having a BRCA1/2 mutation. The specific segments of the sequenced genes distinguishing between BRCA1/2 positive and negative cases, referred to herein as target sequences, were selected from candidate sequences based on a k-fold cross-validation procedure (with k=10). For an individual segment of a gene, a naïve Bayesian (NB) classifier was constructed on the training of k-1 subsets and tested on the other testing subset. For each candidate segment, the NB classifier was trained using the CNV data of that candidate segment as input. The training and testing subsets were then rotated, and the average of the classification errors was used to measure the relevancy of the candidate segment where a lower average of the classification errors corresponded to a more relevant candidate segment. A total of 26,940 candidate segments were evaluated. The evaluated candidate segments were ranked from most relevant (i.e., lowest average of the classification errors) to least relevant (i.e., highest average of the classification errors) Table 3 includes a ranked list of the top 16,383 most relevant candidate segments based on having the lowest mean classification error. The first column of Column 1 of Table 3 ranks the candidate segments, also referred to as “bins”, from the best rank (i.e., lowest rank number and lowest mean classification error) corresponding to the most relevant candidate segment to the worst rank (i.e., highest rank number and highest mean classifications error) corresponding to the least relevant candidate segment. The second column of Table 3 lists names of the bins, where a named gene associated with the bin is identified, or if the bin does not correspond to a particular named gene, the bin is labeled “I.S.” or “intervening sequence”. The third column of Table 3 lists the positive predictive value (PPV) for the bin when using the candidate segment or “bin” alone for classification.

TABLE 3
Ranked list of top 16,383 most relevant candidate segments

| Rank | Name | PPV | Rank | Name | PPV | Rank | Name | PPV |
|---|---|---|---|---|---|---|---|---|
| 1 | I.S. | 0.8111 | 2 | RNF168 | 0.80518 | 3 | KMT2C | 0.80423 |
| 4 | BRCA2 | 0.79654 | 5 | SMAD9 | 0.79535 | 6 | NSD1 | 0.79276 |
| 7 | LYST | 0.78653 | 8 | EGFR | 0.78626 | 9 | I.S. | 0.784 |
| 10 | FOXP1 | 0.78329 | 11 | I.S. | 0.78198 | 12 | NSD1 | 0.7794 |
| 13 | LRP1B | 0.77753 | 14 | ATM | 0.77672 | 15 | I.S. | 0.77586 |
| 16 | I.S. | 0.7747 | 17 | I.S. | 0.77396 | 18 | I.S. | 0.77289 |
| 19 | I.S. | 0.77256 | 20 | I.S. | 0.77256 | 21 | FAT1 | 0.7725 |
| 22 | I.S. | 0.77194 | 23 | MTOR | 0.77152 | 24 | QKI | 0.77054 |
| 25 | I.S. | 0.77042 | 26 | I.S. | 0.77042 | 27 | ASXL1 | 0.77037 |
| 28 | FAT1 | 0.77033 | 29 | FBXW7 | 0.76989 | 30 | ADGRA2 | 0.7698 |
| 31 | I.S. | 0.76942 | 32 | EPHB1 | 0.76906 | 33 | FLT3 | 0.76867 |
| 34 | I.S. | 0.76856 | 35 | I.S. | 0.7684 | 36 | FLI1 | 0.76835 |
| 37 | I.S. | 0.76829 | 38 | BRIP1 | 0.76806 | 39 | I.S. | 0.7679 |
| 40 | I.S. | 0.76758 | 41 | KMT2B | 0.76737 | 42 | I.S. | 0.76701 |
| 43 | CCND2 | 0.76695 | 44 | BRCA2 | 0.76686 | 45 | GATA4 | 0.76659 |
| 46 | ERG | 0.76621 | 47 | LYST | 0.76618 | 48 | SUFU | 0.76618 |
| 49 | RAD51C | 0.76618 | 50 | NTRK3 | 0.76614 | 51 | ARID1B | 0.76597 |
| 52 | I.S. | 0.76592 | 53 | I.S. | 0.76561 | 54 | FAT1 | 0.76552 |
| 55 | SRC | 0.76502 | 56 | I.S. | 0.76487 | 57 | CDK8 | 0.76457 |
| 58 | I.S. | 0.76454 | 59 | I.S. | 0.76454 | 60 | CDH1 | 0.76383 |
| 61 | KMT2C | 0.76362 | 62 | I.S. | 0.7635 | 63 | ARID1B | 0.76341 |
| 64 | FAT1 | 0.76327 | 65 | I.S. | 0.76318 | 66 | I.S. | 0.76309 |
| 67 | CDK8 | 0.76308 | 68 | I.S. | 0.76306 | 69 | IGF1R | 0.763 |
| 70 | I.S. | 0.76273 | 71 | NSD1 | 0.76255 | 72 | SLIT2 | 0.7625 |
| 73 | PRKN | 0.76222 | 74 | CHEK1 | 0.7621 | 75 | FAT1 | 0.76199 |
| 76 | I.S. | 0.76181 | 77 | I.S. | 0.76181 | 78 | I.S. | 0.76164 |
| 79 | ARID1B | 0.76158 | 80 | BRCA2 | 0.76157 | 81 | PIK3C2B | 0.76148 |
| 82 | BRCA2 | 0.76136 | 83 | CSF1R | 0.76062 | 84 | FAT1 | 0.7606 |
| 85 | I.S. | 0.7606 | 86 | I.S. | 0.76009 | 87 | I.S. | 0.75988 |
| 88 | I.S. | 0.75931 | 89 | I.S. | 0.75931 | 90 | I.S. | 0.75931 |
| 91 | I.S. | 0.75931 | 92 | FGF6 | 0.75926 | 93 | ERBB4 | 0.75905 |
| 94 | I.S. | 0.75899 | 95 | CCND2 | 0.75893 | 96 | I.S. | 0.75828 |
| 97 | FAT1 | 0.75791 | 98 | TSC1 | 0.75745 | 99 | PSTPIP1 | 0.75729 |
| 100 | STAT4 | 0.75721 | 101 | PRKN | 0.7572 | 102 | LZTR1 | 0.75713 |
| 103 | I.S. | 0.75697 | 104 | PLCG2 | 0.75691 | 105 | I.S. | 0.75685 |
| 106 | I.S. | 0.7567 | 107 | I.S. | 0.75647 | 108 | FGF14 | 0.75635 |
| 109 | INHBA | 0.75632 | 110 | FLI1 | 0.75616 | 111 | I.S. | 0.75614 |
| 112 | SNCAIP | 0.7561 | 113 | RAD21 | 0.75607 | 114 | I.S. | 0.75566 |
| 115 | CDK8 | 0.75559 | 116 | ERG | 0.75557 | 117 | SMC3 | 0.75545 |
| 118 | I.S. | 0.75516 | 119 | I.S. | 0.75504 | 120 | NSD1 | 0.75486 |
| 121 | I.S. | 0.75483 | 122 | LMO1 | 0.75466 | 123 | I.S. | 0.75445 |
| 124 | I.S. | 0.75433 | 125 | I.S. | 0.75408 | 126 | I.S. | 0.754 |
| 127 | I.S. | 0.75391 | 128 | I.S. | 0.75387 | 129 | I.S. | 0.75387 |
| 130 | I.S. | 0.75379 | 131 | I.S. | 0.75355 | 132 | I.S. | 0.75335 |
| 133 | I.S. | 0.75329 | 134 | LYST | 0.75316 | 135 | I.S. | 0.75316 |
| 136 | BRCA2 | 0.75304 | 137 | TSC1 | 0.75301 | 138 | BRCA2 | 0.75299 |
| 139 | AKT2 | 0.75293 | 140 | I.S. | 0.75272 | 141 | I.S. | 0.75272 |
| 142 | I.S. | 0.7526 | 143 | I.S. | 0.75236 | 144 | ARID1B | 0.75227 |
| 145 | FAT1 | 0.75206 | 146 | KMT2C | 0.75206 | 147 | I.S. | 0.75174 |
| 148 | I.S. | 0.75162 | 149 | I.S. | 0.75162 | 150 | I.S. | 0.75162 |
| 151 | I.S. | 0.75162 | 152 | I.S. | 0.75162 | 153 | KDM5A | 0.75156 |
| 154 | KIF23 | 0.75145 | 155 | I.S. | 0.75129 | 156 | I.S. | 0.75108 |
| 157 | BRCA2 | 0.75091 | 158 | KMT2B | 0.75091 | 159 | SBF2 | 0.75088 |
| 160 | I.S. | 0.75088 | 161 | ADGRA2 | 0.75076 | 162 | I.S. | 0.7507 |
| 163 | QKI | 0.75067 | 164 | EPHA7 | 0.75067 | 165 | I.S. | 0.75058 |
| 166 | RNF168 | 0.75058 | 167 | I.S. | 0.75041 | 168 | I.S. | 0.75034 |
| 169 | I.S. | 0.75034 | 170 | I.S. | 0.75026 | 171 | I.S. | 0.75001 |
| 172 | SETBP1 | 0.74993 | 173 | I.S. | 0.74993 | 174 | I.S. | 0.7499 |
| 175 | I.S. | 0.7499 | 176 | I.S. | 0.74984 | 177 | KMT2C | 0.74966 |
| 178 | I.S. | 0.7496 | 179 | KMT2A | 0.74954 | 180 | I.S. | 0.74918 |
| 181 | KMT2D | 0.74913 | 182 | I.S. | 0.7491 | 183 | SETBP1 | 0.7491 |
| 184 | FAT1 | 0.74907 | 185 | POLE | 0.74889 | 186 | I.S. | 0.74883 |
| 187 | FAT1 | 0.74877 | 188 | KMT2D | 0.74877 | 189 | ADGRA2 | 0.74874 |
| 190 | ARID1B | 0.74872 | 191 | CDK8 | 0.74859 | 192 | I.S. | 0.74856 |
| 193 | KAT6A | 0.74856 | 194 | I.S. | 0.74853 | 195 | I.S. | 0.74845 |
| 196 | I.S. | 0.74845 | 197 | I.S. | 0.74832 | 198 | FLI1 | 0.74832 |
| 199 | NTRK3 | 0.74823 | 200 | I.S. | 0.7482 | 201 | I.S. | 0.7482 |
| 202 | FGF23 | 0.74814 | 203 | I.S. | 0.74812 | 204 | CCND2 | 0.74806 |
| 205 | I.S. | 0.74771 | 206 | I.S. | 0.74755 | 207 | I.S. | 0.74749 |
| 208 | I.S. | 0.74749 | 209 | KMT2A | 0.74749 | 210 | I.S. | 0.74749 |
| 211 | I.S. | 0.74746 | 212 | I.S. | 0.74746 | 213 | I.S. | 0.74735 |
| 214 | I.S. | 0.74725 | 215 | MAP3K14 | 0.74713 | 216 | G6PC3 | 0.74705 |
| 217 | IKZF3 | 0.74684 | 218 | I.S. | 0.74675 | 219 | I.S. | 0.74675 |
| 220 | ROS1 | 0.74675 | 221 | JAK2 | 0.74675 | 222 | JAGN1 | 0.74651 |
| 223 | I.S. | 0.74648 | 224 | BIRC3 | 0.74643 | 225 | I.S. | 0.74639 |
| 226 | I.S. | 0.74639 | 227 | I.S. | 0.74639 | 228 | PSTPIP1 | 0.74639 |
| 229 | I.S. | 0.74625 | 230 | SPTA1 | 0.74624 | 231 | PRDM1 | 0.74612 |
| 232 | I.S. | 0.74607 | 233 | I.S. | 0.74607 | 234 | I.S. | 0.74577 |
| 235 | LIG4 | 0.74576 | 236 | I.S. | 0.74574 | 237 | I.S. | 0.74538 |
| 238 | I.S. | 0.74535 | 239 | I.S. | 0.74532 | 240 | FAT1 | 0.74524 |
| 241 | DDR2 | 0.74517 | 242 | SBF2 | 0.74509 | 243 | I.S. | 0.74503 |
| 244 | CCNE1 | 0.74503 | 245 | I.S. | 0.74503 | 246 | FAT1 | 0.74494 |
| 247 | I.S. | 0.74478 | 248 | I.S. | 0.74467 | 249 | FGF6 | 0.74464 |
| 250 | I.S. | 0.74441 | 251 | I.S. | 0.74437 | 252 | I.S. | 0.74429 |
| 253 | LIG4 | 0.74428 | 254 | NSD1 | 0.74405 | 255 | I.S. | 0.74393 |
| 256 | IGF1R | 0.74384 | 257 | I.S. | 0.74366 | 258 | I.S. | 0.74358 |
| 259 | I.S. | 0.74351 | 260 | SDHA | 0.74345 | 261 | KMT2C | 0.74333 |
| 262 | SLIT2 | 0.74327 | 263 | I.S. | 0.74322 | 264 | I.S. | 0.74322 |
| 265 | I.S. | 0.74322 | 266 | CDC73 | 0.74307 | 267 | ERBB2 | 0.74301 |
| 268 | I.S. | 0.74297 | 269 | SETBP1 | 0.74297 | 270 | I.S. | 0.74289 |
| 271 | I.S. | 0.74286 | 272 | I.S. | 0.74286 | 273 | I.S. | 0.74265 |
| 274 | I.S. | 0.74265 | 275 | MYC | 0.74253 | 276 | IRF2 | 0.74253 |
| 277 | I.S. | 0.74253 | 278 | FAT1 | 0.74247 | 279 | I.S. | 0.74226 |
| 280 | I.S. | 0.7422 | 281 | NPM1 | 0.74215 | 282 | IRF4 | 0.74212 |
| 283 | I.S. | 0.74199 | 284 | CARD11 | 0.74198 | 285 | I.S. | 0.74197 |
| 286 | NLRP3 | 0.74194 | 287 | I.S. | 0.74194 | 288 | I.S. | 0.74191 |
| 289 | I.S. | 0.74185 | 290 | LYST | 0.7418 | 291 | I.S. | 0.74179 |
| 292 | FGFR2 | 0.74167 | 293 | I.S. | 0.74164 | 294 | I.S. | 0.74161 |
| 295 | I.S. | 0.74161 | 296 | I.S. | 0.74161 | 297 | I.S. | 0.74149 |
| 298 | MEN1 | 0.74149 | 299 | I.S. | 0.74128 | 300 | I.S. | 0.7412 |
| 301 | I.S. | 0.7412 | 302 | I.S. | 0.74114 | 303 | I.S. | 0.74108 |
| 304 | I.S. | 0.74105 | 305 | BRCA1 | 0.74102 | 306 | I.S. | 0.74098 |
| 307 | I.S. | 0.74087 | 308 | I.S. | 0.74084 | 309 | I.S. | 0.74084 |
| 310 | JAK3 | 0.74081 | 311 | TET2 | 0.74078 | 312 | EGFR | 0.74078 |
| 313 | I.S. | 0.74075 | 314 | I.S. | 0.74072 | 315 | CRKL | 0.7407 |
| 316 | LYST | 0.74069 | 317 | NSD1 | 0.74055 | 318 | I.S. | 0.74051 |
| 319 | I.S. | 0.74051 | 320 | I.S. | 0.74051 | 321 | GATA2 | 0.74045 |
| 322 | NSD2 | 0.74037 | 323 | PRSS8 | 0.74037 | 324 | I.S. | 0.7403 |
| 325 | SETBP1 | 0.7403 | 326 | NSD2 | 0.74021 | 327 | I.S. | 0.74 |
| 328 | I.S. | 0.73998 | 329 | POLE | 0.73992 | 330 | RAD50 | 0.7398 |
| 331 | I.S. | 0.7398 | 332 | I.S. | 0.7398 | 333 | I.S. | 0.73957 |
| 334 | I.S. | 0.7395 | 335 | I.S. | 0.73947 | 336 | I.S. | 0.73947 |
| 337 | I.S. | 0.73947 | 338 | IRS2 | 0.73935 | 339 | I.S. | 0.73923 |
| 340 | I.S. | 0.73918 | 341 | FAT1 | 0.73917 | 342 | GEN1 | 0.73915 |
| 343 | I.S. | 0.73915 | 344 | KMT2D | 0.73915 | 345 | POLE | 0.73915 |
| 346 | RTEL1 | 0.73915 | 347 | LZTR1 | 0.73906 | 348 | RNF168 | 0.73905 |
| 349 | MDM4 | 0.7389 | 350 | TERF2 | 0.73882 | 351 | APC | 0.7387 |
| 352 | EPHB1 | 0.73869 | 353 | DOT1L | 0.73858 | 354 | NSD1 | 0.73858 |
| 355 | I.S. | 0.73852 | 356 | I.S. | 0.73849 | 357 | FLT4 | 0.73828 |
| 358 | CCND2 | 0.73807 | 359 | CDAN1 | 0.73802 | 360 | U2AF1, | 0.73802 |
| | | | | | | | U2AF1L5 | |
| 361 | NLRP3 | 0.73798 | 362 | I.S. | 0.73778 | 363 | I.S. | 0.73766 |
| 364 | PIK3CA | 0.73763 | 365 | PLCG2 | 0.7376 | 366 | I.S. | 0.73757 |
| 367 | KDM5A | 0.73757 | 368 | I.S. | 0.73745 | 369 | I.S. | 0.73745 |
| 370 | PRKN | 0.73745 | 371 | I.S. | 0.73745 | 372 | I.S. | 0.73742 |
| 373 | FGF14 | 0.73737 | 374 | BRCA2 | 0.73733 | 375 | I.S. | 0.73721 |
| 376 | I.S. | 0.73709 | 377 | KMT2D | 0.73709 | 378 | LYST | 0.73707 |
| 379 | I.S. | 0.73701 | 380 | AKT2 | 0.73701 | 381 | FGFR1 | 0.737 |
| 382 | FLI1 | 0.73695 | 383 | ADGRA2 | 0.73692 | 384 | I.S. | 0.73692 |
| 385 | I.S. | 0.73688 | 386 | I.S. | 0.73688 | 387 | I.S. | 0.73676 |
| 388 | I.S. | 0.73676 | 389 | PRKN | 0.73671 | 390 | JAK2 | 0.73671 |
| 391 | I.S. | 0.73665 | 392 | EPHB1 | 0.73662 | 393 | I.S. | 0.7366 |
| 394 | FLI1 | 0.7365 | 395 | NF1 | 0.73647 | 396 | I.S. | 0.73644 |
| 397 | I.S. | 0.73644 | 398 | I.S. | 0.73639 | 399 | I.S. | 0.73639 |
| 400 | I.S. | 0.73638 | 401 | LIG4 | 0.73638 | 402 | ZNF217 | 0.73632 |
| 403 | I.S. | 0.73632 | 404 | I.S. | 0.7363 | 405 | SLIT2 | 0.73627 |
| 406 | PLCG1 | 0.73626 | 407 | SMAD9 | 0.73621 | 408 | FANCM | 0.73618 |
| 409 | I.S. | 0.73597 | 410 | I.S. | 0.73594 | 411 | I.S. | 0.73569 |
| 412 | KMT2C | 0.73563 | 413 | I.S. | 0.73561 | 414 | I.S. | 0.73553 |
| 415 | ALK | 0.73544 | 416 | FLT1 | 0.73542 | 417 | LYST | 0.73532 |
| 418 | I.S. | 0.73531 | 419 | AKT2 | 0.73525 | 420 | I.S. | 0.7352 |
| 421 | I.S. | 0.7352 | 422 | I.S. | 0.73505 | 423 | PLCG2 | 0.73504 |
| 424 | FLT1 | 0.73501 | 425 | FLT3 | 0.73498 | 426 | I.S. | 0.73495 |
| 427 | I.S. | 0.73495 | 428 | I.S. | 0.73484 | 429 | CHEK1 | 0.73469 |
| 430 | FGF23 | 0.73465 | 431 | I.S. | 0.73463 | 432 | I.S. | 0.73457 |
| 433 | I.S. | 0.73454 | 434 | CDH1 | 0.73449 | 435 | FANCA | 0.73445 |
| 436 | MAP2K4 | 0.73445 | 437 | RET | 0.73439 | 438 | I.S. | 0.73428 |
| 439 | I.S. | 0.73428 | 440 | I.S. | 0.73424 | 441 | I.S. | 0.73416 |
| 442 | CHD4 | 0.73404 | 443 | GRIN2A | 0.73404 | 444 | I.S. | 0.73392 |
| 445 | I.S. | 0.73392 | 446 | I.S. | 0.73388 | 447 | FH | 0.73386 |
| 448 | I.S. | 0.73377 | 449 | RAD21 | 0.73366 | 450 | I.S. | 0.73359 |
| 451 | I.S. | 0.73359 | 452 | I.S. | 0.73359 | 453 | ZNF276, | 0.73357 |
| | | | | | | | FANCA | |
| 454 | I.S. | 0.73356 | 455 | JAK2 | 0.73353 | 456 | I.S. | 0.7335 |
| 457 | IKBKE | 0.73326 | 458 | RAC1 | 0.73326 | 459 | KDM5A | 0.7332 |
| 460 | LIG4 | 0.7332 | 461 | PIK3C2B | 0.73317 | 462 | CTCF | 0.73317 |
| 463 | I.S. | 0.73314 | 464 | I.S. | 0.73293 | 465 | I.S. | 0.73293 |
| 466 | ABL2 | 0.73288 | 467 | I.S. | 0.73282 | 468 | I.S. | 0.73261 |
| 469 | I.S. | 0.73252 | 470 | I.S. | 0.73252 | 471 | I.S. | 0.73249 |
| 472 | POLE | 0.7324 | 473 | ARID1B | 0.73238 | 474 | I.S. | 0.73216 |
| 475 | CUX1 | 0.73211 | 476 | I.S. | 0.73211 | 477 | I.S. | 0.73211 |
| 478 | TSC2 | 0.73211 | 479 | I.S. | 0.73202 | 480 | BRCA2 | 0.73198 |
| 481 | DICER1 | 0.73193 | 482 | FLT3 | 0.73192 | 483 | I.S. | 0.7319 |
| 484 | ESR1 | 0.73186 | 485 | I.S. | 0.73186 | 486 | LYST | 0.73184 |
| 487 | I.S. | 0.73178 | 488 | ERBB2 | 0.73178 | 489 | I.S. | 0.73178 |
| 490 | LYST | 0.73171 | 491 | I.S. | 0.73166 | 492 | I.S. | 0.73161 |
| 493 | KMT2B | 0.73161 | 494 | I.S. | 0.73157 | 495 | I.S. | 0.73154 |
| 496 | I.S. | 0.73154 | 497 | I.S. | 0.73149 | 498 | I.S. | 0.73149 |
| 499 | SLIT2 | 0.73148 | 500 | I.S. | 0.73145 | 501 | I.S. | 0.73145 |
| 502 | PLCG2 | 0.73143 | 503 | I.S. | 0.73142 | 504 | I.S. | 0.73142 |
| 505 | ARID1B | 0.73133 | 506 | I.S. | 0.73121 | 507 | I.S. | 0.73119 |
| 508 | I.S. | 0.73112 | 509 | I.S. | 0.7311 | 510 | EPHA7 | 0.73109 |
| 511 | I.S. | 0.73109 | 512 | LZTR1 | 0.73092 | 513 | I.S. | 0.73074 |
| 514 | ARID1B | 0.73074 | 515 | QKI | 0.73071 | 516 | ERBB2 | 0.73071 |
| 517 | NTRK3 | 0.73071 | 518 | NTRK3 | 0.73051 | 519 | I.S. | 0.7305 |
| 520 | I.S. | 0.73046 | 521 | I.S. | 0.73041 | 522 | I.S. | 0.73041 |
| 523 | FLT1 | 0.73041 | 524 | INPP4B | 0.73038 | 525 | I.S. | 0.73038 |
| 526 | INPP4B | 0.73038 | 527 | LRP1B | 0.7303 | 528 | IKZF3 | 0.7303 |
| 529 | I.S. | 0.73026 | 530 | I.S. | 0.73017 | 531 | DDX11 | 0.73017 |
| 532 | I.S. | 0.73017 | 533 | SETBP1 | 0.73014 | 534 | I.S. | 0.73014 |
| 535 | FAT1 | 0.73005 | 536 | I.S. | 0.73003 | 537 | BCL6 | 0.73 |
| 538 | I.S. | 0.72997 | 539 | LZTR1 | 0.72997 | 540 | EPHA3 | 0.72988 |
| 541 | I.S. | 0.72973 | 542 | I.S. | 0.72961 | 543 | I.S. | 0.72959 |
| 544 | I.S. | 0.72959 | 545 | FAT1 | 0.72956 | 546 | I.S. | 0.7294 |
| 547 | I.S. | 0.7294 | 548 | I.S. | 0.7294 | 549 | PIK3C2B | 0.72934 |
| 550 | KMT2B | 0.72934 | 551 | I.S. | 0.72932 | 552 | I.S. | 0.72932 |
| 553 | KLHL6 | 0.72931 | 554 | I.S. | 0.72919 | 555 | CCNE1 | 0.72919 |
| 556 | I.S. | 0.72907 | 557 | GSK3B | 0.72901 | 558 | EGFR | 0.72901 |
| 559 | NOTCH3 | 0.72893 | 560 | RPTOR | 0.72875 | 561 | I.S. | 0.72872 |
| 562 | I.S. | 0.72872 | 563 | I.S. | 0.72872 | 564 | I.S. | 0.72872 |
| 565 | I.S. | 0.72869 | 566 | SYK | 0.72869 | 567 | I.S. | 0.72869 |
| 568 | I.S. | 0.72863 | 569 | I.S. | 0.7286 | 570 | CSF1R | 0.7286 |
| 571 | I.S. | 0.72857 | 572 | LZTR1 | 0.72852 | 573 | KMT2C | 0.72839 |
| 574 | I.S. | 0.72836 | 575 | I.S. | 0.72836 | 576 | MAP3K1 | 0.72836 |
| 577 | I.S. | 0.72836 | 578 | I.S. | 0.72836 | 579 | TOP2A | 0.72833 |
| 580 | I.S. | 0.72833 | 581 | I.S. | 0.72828 | 582 | NTRK3 | 0.72828 |
| 583 | FRS2 | 0.72824 | 584 | I.S. | 0.72822 | 585 | I.S. | 0.72807 |
| 586 | I.S. | 0.72803 | 587 | I.S. | 0.72803 | 588 | I.S. | 0.72803 |
| 589 | I.S. | 0.728 | 590 | I.S. | 0.72797 | 591 | ATR | 0.72795 |
| 592 | I.S. | 0.72783 | 593 | QKI | 0.72783 | 594 | MET | 0.72782 |
| 595 | I.S. | 0.72779 | 596 | NF1 | 0.72779 | 597 | LYST | 0.72777 |
| 598 | DDX41 | 0.72773 | 599 | I.S. | 0.72771 | 600 | KMT2B | 0.72771 |
| 601 | I.S. | 0.72769 | 602 | I.S. | 0.72767 | 603 | SMAD9 | 0.72762 |
| 604 | I.S. | 0.72762 | 605 | — | 0.72759 | 606 | CTNNA1 | 0.72757 |
| 607 | ABL2 | 0.72751 | 608 | I.S. | 0.7275 | 609 | I.S. | 0.72745 |
| 610 | I.S. | 0.72741 | 611 | BRCA1 | 0.72738 | 612 | I.S. | 0.72738 |
| 613 | I.S. | 0.72732 | 614 | FAT1 | 0.72729 | 615 | I.S. | 0.72726 |
| 616 | FAT1 | 0.72714 | 617 | KMT2C | 0.72708 | 618 | I.S. | 0.72708 |
| 619 | KMT2C | 0.72705 | 620 | I.S. | 0.727 | 621 | I.S. | 0.72696 |
| 622 | I.S. | 0.72693 | 623 | I.S. | 0.72688 | 624 | I.S. | 0.72685 |
| 625 | RNF168 | 0.72682 | 626 | IKBKE | 0.72678 | 627 | I.S. | 0.72672 |
| 628 | ADA | 0.72663 | 629 | EGFR | 0.72655 | 630 | I.S. | 0.72655 |
| 631 | I.S. | 0.72643 | 632 | SUZ12 | 0.72634 | 633 | LZTR1 | 0.72634 |
| 634 | PREX2 | 0.72631 | 635 | I.S. | 0.72626 | 636 | I.S. | 0.72622 |
| 637 | I.S. | 0.72622 | 638 | I.S. | 0.72622 | 639 | I.S. | 0.72622 |
| 640 | RTEL1 | 0.72619 | 641 | BRD4 | 0.72617 | 642 | BCR | 0.72616 |
| 643 | I.S. | 0.72598 | 644 | MDM2 | 0.72598 | 645 | I.S. | 0.7259 |
| 646 | I.S. | 0.7259 | 647 | ZNF276, | 0.7259 | 648 | GATA3 | 0.72589 |
| | | | | FANCA | | | | |
| 649 | RNF168 | 0.72581 | 650 | I.S. | 0.72565 | 651 | MAP2K4 | 0.72565 |
| 652 | KMT2C | 0.7256 | 653 | FGFR2 | 0.72557 | 654 | I.S. | 0.72551 |
| 655 | I.S. | 0.72551 | 656 | I.S. | 0.72551 | 657 | SPEN | 0.7253 |
| 658 | LIG4 | 0.72527 | 659 | BRCA2 | 0.72523 | 660 | I.S. | 0.72519 |
| 661 | I.S. | 0.72515 | 662 | I.S. | 0.72515 | 663 | I.S. | 0.72513 |
| 664 | I.S. | 0.72494 | 665 | I.S. | 0.72494 | 666 | CHEK1 | 0.72491 |
| 667 | LZTR1 | 0.72486 | 668 | LZTR1 | 0.72486 | 669 | ERBB3 | 0.7248 |
| 670 | KMT2D | 0.72471 | 671 | HNF1A | 0.72465 | 672 | LIG4 | 0.72461 |
| 673 | I.S. | 0.72453 | 674 | NSD1 | 0.72453 | 675 | POLE | 0.72453 |
| 676 | IGF1R | 0.72453 | 677 | KDM5A | 0.72452 | 678 | TBX3 | 0.7245 |
| 679 | I.S. | 0.72445 | 680 | I.S. | 0.72441 | 681 | I.S. | 0.72438 |
| 682 | ARFRP1 | 0.72433 | 683 | APC | 0.72432 | 684 | BRCA2 | 0.72426 |
| 685 | ERBB2 | 0.7242 | 686 | LZTR1 | 0.7242 | 687 | ERBB3 | 0.72417 |
| 688 | SETBP1 | 0.72417 | 689 | AKT2 | 0.72412 | 690 | I.S. | 0.72409 |
| 691 | BCR | 0.72409 | 692 | I.S. | 0.72405 | 693 | I.S. | 0.72403 |
| 694 | I.S. | 0.72403 | 695 | I.S. | 0.72403 | 696 | I.S. | 0.72403 |
| 697 | I.S. | 0.72399 | 698 | KMT2A | 0.72397 | 699 | FANCM | 0.7239 |
| 700 | I.S. | 0.72388 | 701 | FGF6 | 0.72387 | 702 | I.S. | 0.72384 |
| 703 | BCL2L1, | 0.72382 | 704 | KMT2D | 0.72381 | 705 | I.S. | 0.72379 |
| | ABALON | | | | | | | |
| 706 | I.S. | 0.72379 | 707 | JAK2 | 0.72379 | 708 | I.S. | 0.72376 |
| 709 | I.S. | 0.72373 | 710 | FLT1 | 0.7237 | 711 | U2AF2 | 0.7237 |
| 712 | SPEN | 0.72355 | 713 | I.S. | 0.72349 | 714 | NOTCH3 | 0.72344 |
| 715 | PLCG1 | 0.72343 | 716 | I.S. | 0.72341 | 717 | I.S. | 0.72334 |
| 718 | FAT1 | 0.72334 | 719 | I.S. | 0.72334 | 720 | FANCI | 0.72334 |
| 721 | I.S. | 0.72328 | 722 | I.S. | 0.72325 | 723 | FBXW7 | 0.72319 |
| 724 | I.S. | 0.72313 | 725 | FAT1 | 0.72313 | 726 | FAT1 | 0.7231 |
| 727 | SMAD4 | 0.72305 | 728 | CSF1R | 0.72304 | 729 | ERBB2 | 0.72302 |
| 730 | I.S. | 0.72302 | 731 | ZNF217 | 0.72296 | 732 | FAT1 | 0.72289 |
| 733 | FRS2 | 0.72284 | 734 | NSD2 | 0.72283 | 735 | RAD51C | 0.72281 |
| 736 | I.S. | 0.72281 | 737 | FANCM | 0.72272 | 738 | I.S. | 0.72272 |
| 739 | PSTPIP1 | 0.72267 | 740 | I.S. | 0.72263 | 741 | SETBP1 | 0.72259 |
| 742 | ERG | 0.72257 | 743 | SLIT2 | 0.72248 | 744 | I.S. | 0.72248 |
| 745 | I.S. | 0.72248 | 746 | I.S. | 0.72244 | 747 | CIC | 0.72243 |
| 748 | ERBB2 | 0.72242 | 749 | GSK3B | 0.72239 | 750 | ALK | 0.72236 |
| 751 | IGF1R | 0.72229 | 752 | I.S. | 0.72228 | 753 | I.S. | 0.72223 |
| 754 | I.S. | 0.72222 | 755 | PIK3C2B | 0.72218 | 756 | PDGFRA | 0.72215 |
| 757 | CUX1 | 0.72215 | 758 | NBN | 0.72215 | 759 | FLT1 | 0.72215 |
| 760 | FANCI | 0.72215 | 761 | TOP2A | 0.72215 | 762 | CCNE1 | 0.72213 |
| 763 | PLCG2 | 0.72209 | 764 | ATM | 0.72209 | 765 | KEL | 0.72206 |
| 766 | PIK3CB | 0.72201 | 767 | FGFR4 | 0.72198 | 768 | I.S. | 0.72198 |
| 769 | TBX3 | 0.72185 | 770 | I.S. | 0.72177 | 771 | I.S. | 0.72177 |
| 772 | I.S. | 0.72177 | 773 | I.S. | 0.72174 | 774 | I.S. | 0.72171 |
| 775 | I.S. | 0.72171 | 776 | I.S. | 0.72171 | 777 | KAT6A | 0.72158 |
| 778 | PRKN, | 0.72157 | 779 | ERG | 0.72152 | 780 | PALB2 | 0.7215 |
| | PACRG | | | | | | | |
| 781 | I.S. | 0.72144 | 782 | I.S. | 0.72141 | 783 | I.S. | 0.72138 |
| 784 | I.S. | 0.72136 | 785 | ATG2B | 0.72135 | 786 | PIK3C2B | 0.72132 |
| 787 | I.S. | 0.72132 | 788 | NTRK3 | 0.72132 | 789 | FLCN | 0.72132 |
| 790 | I.S. | 0.72129 | 791 | RTEL1 | 0.72129 | 792 | CIC | 0.72124 |
| 793 | PDGFRA | 0.7212 | 794 | I.S. | 0.72119 | 795 | I.S. | 0.72108 |
| 796 | POLE | 0.72108 | 797 | I.S. | 0.72103 | 798 | MYCL | 0.721 |
| 799 | BRCA2 | 0.721 | 800 | I.S. | 0.72091 | 801 | I.S. | 0.72088 |
| 802 | KMT2C | 0.72087 | 803 | I.S. | 0.72078 | 804 | I.S. | 0.72075 |
| 805 | I.S. | 0.72075 | 806 | I.S. | 0.72073 | 807 | I.S. | 0.72067 |
| 808 | I.S. | 0.72067 | 809 | FAT1 | 0.72067 | 810 | I.S. | 0.72067 |
| 811 | I.S. | 0.72067 | 812 | FAT1 | 0.72063 | 813 | I.S. | 0.72063 |
| 814 | I.S. | 0.72059 | 815 | RARA | 0.72058 | 816 | I.S. | 0.72054 |
| 817 | I.S. | 0.72053 | 818 | I.S. | 0.72049 | 819 | I.S. | 0.72046 |
| 820 | PTCH1 | 0.72042 | 821 | I.S. | 0.72042 | 822 | CUL3 | 0.72037 |
| 823 | ATM | 0.72037 | 824 | FLI1 | 0.72037 | 825 | PLCG2 | 0.72037 |
| 826 | CDAN1 | 0.72034 | 827 | LZTR1 | 0.72028 | 828 | SOCS1 | 0.72028 |
| 829 | LYST | 0.72025 | 830 | ZNF217 | 0.72025 | 831 | I.S. | 0.7202 |
| 832 | PRKCI | 0.72012 | 833 | I.S. | 0.72001 | 834 | I.S. | 0.71998 |
| 835 | I.S. | 0.71996 | 836 | I.S. | 0.71996 | 837 | I.S. | 0.71996 |
| 838 | I.S. | 0.71996 | 839 | I.S. | 0.7199 | 840 | I.S. | 0.71989 |
| 841 | I.S. | 0.71987 | 842 | I.S. | 0.71987 | 843 | PIK3C2B | 0.71982 |
| 844 | I.S. | 0.71977 | 845 | I.S. | 0.71977 | 846 | I.S. | 0.71971 |
| 847 | CDK8 | 0.71971 | 848 | BLM | 0.71971 | 849 | I.S. | 0.71971 |
| 850 | APC | 0.71969 | 851 | CDK4, | 0.71969 | 852 | I.S. | 0.71967 |
| | | | | MIR6759 | | | | |
| 853 | I.S. | 0.71963 | 854 | I.S. | 0.71963 | 855 | I.S. | 0.71951 |
| 856 | ERBB3 | 0.71951 | 857 | I.S. | 0.71943 | 858 | SETBP1 | 0.71942 |
| 859 | I.S. | 0.71939 | 860 | I.S. | 0.71939 | 861 | SMAD4 | 0.71939 |
| 862 | AURKB | 0.71937 | 863 | I.S. | 0.71936 | 864 | ATM | 0.71935 |
| 865 | DDX11 | 0.7193 | 866 | KMT2D | 0.7193 | 867 | I.S. | 0.71928 |
| 868 | I.S. | 0.71927 | 869 | PAX5 | 0.71927 | 870 | ZBTB2 | 0.71924 |
| 871 | I.S. | 0.71924 | 872 | CCNE1 | 0.71922 | 873 | ASXL1 | 0.71915 |
| 874 | BRCA2 | 0.71906 | 875 | ADGRA2 | 0.71903 | 876 | I.S. | 0.71903 |
| 877 | I.S. | 0.71897 | 878 | I.S. | 0.71897 | 879 | I.S. | 0.71895 |
| 880 | — | 0.71895 | 881 | I.S. | 0.71894 | 882 | I.S. | 0.71894 |
| 883 | I.S. | 0.71886 | 884 | CCND2 | 0.71886 | 885 | ERBB2 | 0.71885 |
| 886 | FAT1 | 0.71874 | 887 | I.S. | 0.71873 | 888 | I.S. | 0.71865 |
| 889 | I.S. | 0.71865 | 890 | MYCL | 0.71856 | 891 | I.S. | 0.71853 |
| 892 | KMT2C | 0.71852 | 893 | KDM5A | 0.71852 | 894 | XPO1 | 0.71841 |
| 895 | I.S. | 0.7184 | 896 | LYST | 0.71838 | 897 | FANCF | 0.71836 |
| 898 | I.S. | 0.71832 | 899 | PIK3C2B | 0.71826 | 900 | NSD2 | 0.71826 |
| 901 | I.S. | 0.71815 | 902 | I.S. | 0.71803 | 903 | I.S. | 0.71796 |
| 904 | ASXL1 | 0.71794 | 905 | I.S. | 0.71791 | 906 | SDHA | 0.71785 |
| 907 | I.S. | 0.71782 | 908 | PIK3C2B | 0.71778 | 909 | PIK3C2B | 0.71773 |
| 910 | I.S. | 0.7177 | 911 | BRCA2 | 0.71769 | 912 | PIK3CA | 0.71758 |
| 913 | CHEK1 | 0.71758 | 914 | I.S. | 0.71749 | 915 | I.S. | 0.71749 |
| 916 | I.S. | 0.71746 | 917 | I.S. | 0.71746 | 918 | GLI1 | 0.71741 |
| 919 | LYST | 0.71734 | 920 | I.S. | 0.71728 | 921 | DNMT3A | 0.71725 |
| 922 | I.S. | 0.71725 | 923 | I.S. | 0.71725 | 924 | NTRK3 | 0.7172 |
| 925 | CSF3R | 0.71717 | 926 | I.S. | 0.71717 | 927 | I.S. | 0.71713 |
| 928 | I.S. | 0.71713 | 929 | I.S. | 0.71713 | 930 | FLT3 | 0.71704 |
| 931 | I.S. | 0.71699 | 932 | I.S. | 0.71692 | 933 | I.S. | 0.71692 |
| 934 | FRS2 | 0.71692 | 935 | AXL | 0.71687 | 936 | CSF3R | 0.71684 |
| 937 | I.S. | 0.71681 | 938 | I.S. | 0.71675 | 939 | ARID1B | 0.71675 |
| 940 | I.S. | 0.71668 | 941 | PLCG1 | 0.71666 | 942 | EMSY | 0.71663 |
| 943 | I.S. | 0.71656 | 944 | I.S. | 0.71656 | 945 | KMT2C | 0.71651 |
| 946 | I.S. | 0.71651 | 947 | I.S. | 0.71651 | 948 | POLE | 0.7165 |
| 949 | I.S. | 0.71648 | 950 | I.S. | 0.71646 | 951 | I.S. | 0.71642 |
| 952 | GNAS | 0.71642 | 953 | I.S. | 0.71639 | 954 | NTRK2 | 0.71639 |
| 955 | I.S. | 0.71634 | 956 | MPL | 0.71627 | 957 | CUX1 | 0.71627 |
| 958 | SBF2 | 0.71627 | 959 | KEL | 0.71627 | 960 | I.S. | 0.71621 |
| 961 | DDR2 | 0.71619 | 962 | I.S. | 0.71618 | 963 | PRKN | 0.71618 |
| 964 | PRKCI | 0.71618 | 965 | LYST | 0.71616 | 966 | PLCG2 | 0.71615 |
| 967 | I.S. | 0.71615 | 968 | FLT1 | 0.71614 | 969 | EGFR | 0.71609 |
| 970 | PLCG1 | 0.71607 | 971 | RAF1 | 0.71606 | 972 | IDH2 | 0.71594 |
| 973 | DDX11 | 0.71593 | 974 | GATA4 | 0.71585 | 975 | EXO1 | 0.7158 |
| 976 | BCL2L1, | 0.7158 | 977 | I.S. | 0.71577 | 978 | FRS2 | 0.71576 |
| | ABALON | | | | | | | |
| 979 | TERT | 0.71573 | 980 | RAD51B | 0.71573 | 981 | LYST | 0.7157 |
| 982 | EGLN1 | 0.71568 | 983 | I.S. | 0.71565 | 984 | TOP2A | 0.71565 |
| 985 | I.S. | 0.71564 | 986 | EPHB1 | 0.71562 | 987 | RPTOR | 0.71544 |
| 988 | LPIN2 | 0.71544 | 989 | I.S. | 0.71536 | 990 | NF1 | 0.71535 |
| 991 | I.S. | 0.71532 | 992 | I.S. | 0.71521 | 993 | CRKL | 0.71515 |
| 994 | BIRC3 | 0.71515 | 995 | I.S. | 0.71511 | 996 | DKC1 | 0.71506 |
| 997 | GATA6 | 0.71502 | 998 | MAGI2 | 0.71502 | 999 | I.S. | 0.715 |
| 1000 | SOCS1 | 0.715 | 1001 | I.S. | 0.71497 | 1002 | PREX2 | 0.71493 |
| 1003 | KMT2A | 0.71487 | 1004 | ERBB2 | 0.71485 | 1005 | PIK3C2B | 0.71481 |
| 1006 | PRKN | 0.71466 | 1007 | I.S. | 0.71465 | 1008 | SLIT2 | 0.71464 |
| 1009 | LYST | 0.71448 | 1010 | FLCN | 0.71448 | 1011 | SDHC | 0.71446 |
| 1012 | TET2 | 0.71445 | 1013 | DDR2 | 0.71443 | 1014 | KMT2D | 0.7144 |
| 1015 | LIG4 | 0.71434 | 1016 | KDM5A | 0.71433 | 1017 | I.S. | 0.71431 |
| 1018 | I.S. | 0.71425 | 1019 | I.S. | 0.71422 | 1020 | I.S. | 0.71416 |
| 1021 | I.S. | 0.71415 | 1022 | LYST | 0.7141 | 1023 | I.S. | 0.71402 |
| 1024 | SLIT2 | 0.71401 | 1025 | I.S. | 0.71401 | 1026 | I.S. | 0.71383 |
| 1027 | I.S. | 0.7138 | 1028 | I.S. | 0.71375 | 1029 | KMT2C | 0.71375 |
| 1030 | NTRK3 | 0.71375 | 1031 | PLCG2 | 0.71369 | 1032 | DDX41 | 0.71366 |
| 1033 | RAD21 | 0.71366 | 1034 | I.S. | 0.7136 | 1035 | I.S. | 0.7136 |
| 1036 | FLT3 | 0.7136 | 1037 | FGFR2 | 0.71359 | 1038 | ARFRP1 | 0.71351 |
| 1039 | CTNNB1 | 0.71342 | 1040 | FGFR3 | 0.71339 | 1041 | KAT6A | 0.71339 |
| 1042 | SETBP1 | 0.71335 | 1043 | I.S. | 0.7133 | 1044 | FAT1 | 0.7133 |
| 1045 | I.S. | 0.71327 | 1046 | I.S. | 0.71319 | 1047 | EGFR | 0.71312 |
| 1048 | ACTA2, FAS | 0.71309 | 1049 | I.S. | 0.71306 | 1050 | PMS2 | 0.71306 |
| 1051 | POLE | 0.71306 | 1052 | I.S. | 0.71306 | 1053 | I.S. | 0.71306 |
| 1054 | I.S. | 0.713 | 1055 | I.S. | 0.71298 | 1056 | FAT1 | 0.71297 |
| 1057 | ERG | 0.71295 | 1058 | FGF23 | 0.71292 | 1059 | I.S. | 0.71292 |
| 1060 | FAT1 | 0.71288 | 1061 | GLI2 | 0.71288 | 1062 | DAXX | 0.7128 |
| 1063 | SPTA1 | 0.71279 | 1064 | STAT6 | 0.71279 | 1065 | DOT1L | 0.71273 |
| 1066 | I.S. | 0.71273 | 1067 | I.S. | 0.71273 | 1068 | I.S. | 0.71271 |
| 1069 | ARID1B | 0.71271 | 1070 | BRCA2 | 0.71271 | 1071 | KMT2C | 0.71267 |
| 1072 | I.S. | 0.71265 | 1073 | EGLN1 | 0.71248 | 1074 | NSD1 | 0.71244 |
| 1075 | I.S. | 0.71241 | 1076 | EGLN1 | 0.71238 | 1077 | I.S. | 0.71235 |
| 1078 | FGFR4 | 0.71235 | 1079 | CIC | 0.71235 | 1080 | I.S. | 0.71229 |
| 1081 | I.S. | 0.71229 | 1082 | WT1 | 0.71223 | 1083 | — | 0.71214 |
| 1084 | I.S. | 0.71205 | 1085 | FAT1 | 0.71202 | 1086 | I.S. | 0.71202 |
| 1087 | PLCG1, | 0.71199 | 1088 | LYST | 0.71197 | 1089 | I.S. | 0.71194 |
| | MIR6871 | | | | | | | |
| 1090 | JAURKC | 0.71191 | 1091 | MRE11 | 0.7119 | 1092 | POLE | 0.7119 |
| 1093 | I.S. | 0.71182 | 1094 | I.S. | 0.71178 | 1095 | I.S. | 0.71169 |
| 1096 | I.S. | 0.71167 | 1097 | I.S. | 0.71161 | 1098 | I.S. | 0.71159 |
| 1099 | SBF2 | 0.71158 | 1100 | PLCG1 | 0.71158 | 1101 | FLT1 | 0.71158 |
| 1102 | PLCG2 | 0.71155 | 1103 | CRLF2 | 0.71149 | 1104 | BRIP1 | 0.7114 |
| 1105 | I.S. | 0.71137 | 1106 | I.S. | 0.71137 | 1107 | I.S. | 0.71133 |
| 1108 | I.S. | 0.71133 | 1109 | CTNNB1 | 0.71128 | 1110 | CEBPA | 0.71126 |
| 1111 | I.S. | 0.71125 | 1112 | I.S. | 0.71125 | 1113 | CCND2 | 0.71119 |
| 1114 | KMT2B | 0.71119 | 1115 | VEGFA | 0.71119 | 1116 | I.S. | 0.71117 |
| 1117 | CTNNA1 | 0.71113 | 1118 | HRAS | 0.71113 | 1119 | I.S. | 0.71104 |
| 1120 | I.S. | 0.71104 | 1121 | KMT2D | 0.711 | 1122 | LYST | 0.71098 |
| 1123 | SLIT2 | 0.71095 | 1124 | FGF6 | 0.71095 | 1125 | PIM1 | 0.71092 |
| 1126 | MCL1 | 0.7109 | 1127 | TET2 | 0.71083 | 1128 | BRCA2 | 0.7108 |
| 1129 | I.S. | 0.71077 | 1130 | I.S. | 0.71075 | 1131 | I.S. | 0.71066 |
| 1132 | I.S. | 0.71062 | 1133 | GNAS | 0.71061 | 1134 | BRCA2 | 0.7106 |
| 1135 | RPTOR | 0.71056 | 1136 | ASXL1 | 0.71054 | 1137 | FGFR4 | 0.71051 |
| 1138 | BRCA2 | 0.7105 | 1139 | I.S. | 0.71046 | 1140 | EPHB1 | 0.71045 |
| 1141 | I.S. | 0.71041 | 1142 | ATG2B | 0.71041 | 1143 | FANCE | 0.71039 |
| 1144 | SFTA3, | 0.71039 | 1145 | PLCG1 | 0.71033 | 1146 | NTRK3 | 0.7103 |
| | NKX2-1 | | | | | | | |
| 1147 | CHD2 | 0.7103 | 1148 | I.S. | 0.71027 | 1149 | LZTR1 | 0.71024 |
| 1150 | I.S. | 0.71016 | 1151 | I.S. | 0.71015 | 1152 | SPEN | 0.71013 |
| 1153 | CCNE1 | 0.71013 | 1154 | PMS1 | 0.71009 | 1155 | SNCAIP | 0.71009 |
| 1156 | I.S. | 0.71006 | 1157 | PRKN | 0.71005 | 1158 | RAD50 | 0.71 |
| 1159 | IGF1R | 0.71 | 1160 | I.S. | 0.71 | 1161 | ERBB2 | 0.70997 |
| 1162 | GLI2 | 0.70997 | 1163 | I.S. | 0.70993 | 1164 | CD79A | 0.70992 |
| 1165 | JAK2 | 0.70988 | 1166 | I.S. | 0.7098 | 1167 | I.S. | 0.7098 |
| 1168 | FAT1 | 0.70977 | 1169 | GATA4 | 0.70977 | 1170 | I.S. | 0.70977 |
| 1171 | I.S. | 0.70977 | 1172 | ABL1 | 0.70973 | 1173 | I.S. | 0.70966 |
| 1174 | I.S. | 0.70956 | 1175 | FLI1 | 0.7095 | 1176 | GNA13 | 0.70947 |
| 1177 | EXO1 | 0.70944 | 1178 | I.S. | 0.70944 | 1179 | I.S. | 0.70944 |
| 1180 | I.S. | 0.70942 | 1181 | FAT1 | 0.70941 | 1182 | I.S. | 0.70938 |
| 1183 | I.S. | 0.70923 | 1184 | I.S. | 0.70923 | 1185 | I.S. | 0.70923 |
| 1186 | I.S. | 0.70923 | 1187 | SMAD2 | 0.70923 | 1188 | CTCF | 0.70917 |
| 1189 | SLIT2 | 0.70915 | 1190 | I.S. | 0.70911 | 1191 | I.S. | 0.70909 |
| 1192 | KLLN | 0.70909 | 1193 | I.S. | 0.70908 | 1194 | RTEL1 | 0.70906 |
| 1195 | KDM5A | 0.70905 | 1196 | I.S. | 0.709 | 1197 | I.S. | 0.70899 |
| 1198 | GATA4 | 0.70899 | 1199 | TSC2 | 0.70897 | 1200 | I.S. | 0.7089 |
| 1201 | BRCA1 | 0.7089 | 1202 | CCNE1 | 0.70885 | 1203 | STAT6 | 0.70884 |
| 1204 | I.S. | 0.70882 | 1205 | I.S. | 0.70879 | 1206 | LPIN2 | 0.70873 |
| 1207 | KMT2B | 0.70866 | 1208 | I.S. | 0.70864 | 1209 | GLI2 | 0.70861 |
| 1210 | I.S. | 0.7086 | 1211 | GATA4 | 0.7086 | 1212 | KAT6A | 0.7086 |
| 1213 | IRF4 | 0.70858 | 1214 | NUP93 | 0.70858 | 1215 | I.S. | 0.70857 |
| 1216 | NSD2 | 0.70852 | 1217 | I.S. | 0.70852 | 1218 | INPP4B | 0.70852 |
| 1219 | SLIT2 | 0.70849 | 1220 | I.S. | 0.70849 | 1221 | SETBP1 | 0.70849 |
| 1222 | LIG4 | 0.70848 | 1223 | I.S. | 0.70837 | 1224 | LZTR1 | 0.70828 |
| 1225 | — | 0.70828 | 1226 | I.S. | 0.70825 | 1227 | SMAD4 | 0.70822 |
| 1228 | CUX1 | 0.70819 | 1229 | I.S. | 0.70819 | 1230 | ABL1 | 0.70816 |
| 1231 | I.S. | 0.70816 | 1232 | I.S. | 0.70816 | 1233 | I.S. | 0.70811 |
| 1234 | I.S. | 0.70808 | 1235 | I.S. | 0.70807 | 1236 | I.S. | 0.70804 |
| 1237 | SPTA1 | 0.70802 | 1238 | PRKN | 0.70795 | 1239 | I.S. | 0.70793 |
| 1240 | SMAD4 | 0.70787 | 1241 | PIK3CB | 0.70786 | 1242 | RICTOR | 0.70786 |
| 1243 | I.S. | 0.70786 | 1244 | I.S. | 0.70778 | 1245 | MPL | 0.70775 |
| 1246 | CDK12 | 0.70769 | 1247 | IGF1R | 0.70763 | 1248 | KMT2B | 0.70763 |
| 1249 | I.S. | 0.70759 | 1250 | I.S. | 0.70757 | 1251 | FLT3 | 0.70756 |
| 1252 | TNFAIP3 | 0.70754 | 1253 | I.S. | 0.70754 | 1254 | I.S. | 0.70754 |
| 1255 | MDM4 | 0.70751 | 1256 | I.S. | 0.70751 | 1257 | I.S. | 0.7075 |
| 1258 | I.S. | 0.70748 | 1259 | KMT2C | 0.70748 | 1260 | I.S. | 0.70742 |
| 1261 | I.S. | 0.70742 | 1262 | BAP1 | 0.70737 | 1263 | TET2 | 0.70736 |
| 1264 | SBF2 | 0.70733 | 1265 | FLI1 | 0.70733 | 1266 | I.S. | 0.7073 |
| 1267 | I.S. | 0.7073 | 1268 | MTOR | 0.70729 | 1269 | I.S. | 0.70729 |
| 1270 | I.S. | 0.70724 | 1271 | CTCF | 0.70721 | 1272 | I.S. | 0.70721 |
| 1273 | ADGRA2 | 0.7072 | 1274 | PRKCI | 0.70718 | 1275 | I.S. | 0.70707 |
| 1276 | I.S. | 0.70704 | 1277 | FANCA | 0.70704 | 1278 | I.S. | 0.707 |
| 1279 | I.S. | 0.70699 | 1280 | NF1 | 0.70697 | 1281 | LMO1 | 0.70692 |
| 1282 | TBX3 | 0.70691 | 1283 | I.S. | 0.70689 | 1284 | LYST | 0.70688 |
| 1285 | I.S. | 0.70685 | 1286 | CIC | 0.70684 | 1287 | BLM | 0.7068 |
| 1288 | I.S. | 0.70677 | 1289 | CTC1 | 0.70677 | 1290 | I.S. | 0.70671 |
| 1291 | I.S. | 0.70671 | 1292 | LRP1B | 0.70668 | 1293 | I.S. | 0.70668 |
| 1294 | RTEL1 | 0.70668 | 1295 | ALK | 0.70656 | 1296 | TBX3 | 0.7065 |
| 1297 | EPHB1 | 0.70647 | 1298 | I.S. | 0.70647 | 1299 | LYST | 0.70641 |
| 1300 | I.S. | 0.70638 | 1301 | PLCG2 | 0.70638 | 1302 | I.S. | 0.70638 |
| 1303 | I.S. | 0.70636 | 1304 | I.S. | 0.70635 | 1305 | CDK12 | 0.70632 |
| 1306 | NTRK2 | 0.70622 | 1307 | I.S. | 0.70614 | 1308 | ERBB4 | 0.70614 |
| 1309 | I.S. | 0.70614 | 1310 | I.S. | 0.70614 | 1311 | I.S. | 0.70614 |
| 1312 | NF2 | 0.70612 | 1313 | KLC1, | 0.7061 | 1314 | RTEL1 | 0.7061 |
| | | | | XRCC3 | | | | |
| 1315 | I.S. | 0.70606 | 1316 | I.S. | 0.70602 | 1317 | I.S. | 0.70602 |
| 1318 | IKBKE | 0.706 | 1319 | SLIT2 | 0.706 | 1320 | FANCM | 0.706 |
| 1321 | I.S. | 0.70594 | 1322 | I.S. | 0.70582 | 1323 | I.S. | 0.70581 |
| 1324 | PBRM1 | 0.70581 | 1325 | ZBTB2 | 0.70581 | 1326 | I.S. | 0.70581 |
| 1327 | I.S. | 0.70581 | 1328 | BCL2L1 | 0.70579 | 1329 | CSF1R | 0.70578 |
| 1330 | I.S. | 0.70571 | 1331 | I.S. | 0.7057 | 1332 | I.S. | 0.7057 |
| 1333 | FAT1 | 0.70569 | 1334 | TGFBR2 | 0.70566 | 1335 | I.S. | 0.70548 |
| 1336 | I.S. | 0.70548 | 1337 | ADA | 0.70546 | 1338 | RAD51B | 0.7054 |
| 1339 | CBL | 0.70537 | 1340 | I.S. | 0.70534 | 1341 | AKT2 | 0.70534 |
| 1342 | I.S. | 0.70531 | 1343 | PIK3CA | 0.70531 | 1344 | I.S. | 0.70531 |
| 1345 | I.S. | 0.70531 | 1346 | DDR2 | 0.70529 | 1347 | I.S. | 0.70528 |
| 1348 | I.S. | 0.70528 | 1349 | I.S. | 0.70528 | 1350 | KLHL6 | 0.70525 |
| 1351 | CIC | 0.70523 | 1352 | KMT2B | 0.7052 | 1353 | FAT1 | 0.70518 |
| 1354 | I.S. | 0.70518 | 1355 | SOX2-OT, | 0.70513 | 1356 | I.S. | 0.7051 |
| | | | | SOX2 | | | | |
| 1357 | I.S. | 0.7051 | 1358 | I.S. | 0.70507 | 1359 | I.S. | 0.70507 |
| 1360 | I.S. | 0.70502 | 1361 | GLI2 | 0.7049 | 1362 | NSD1 | 0.7049 |
| 1363 | I.S. | 0.7049 | 1364 | I.S. | 0.70486 | 1365 | APC | 0.70483 |
| 1366 | TSHR | 0.70477 | 1367 | SETBP1 | 0.70477 | 1368 | FANCM | 0.70474 |
| 1369 | I.S. | 0.70474 | 1370 | LYST | 0.70472 | 1371 | I.S. | 0.70469 |
| 1372 | FAT1 | 0.70468 | 1373 | ZNF703 | 0.70466 | 1374 | SMAD9 | 0.70466 |
| 1375 | ATG2B | 0.70465 | 1376 | I.S. | 0.70463 | 1377 | SUFU | 0.70463 |
| 1378 | SF3B1 | 0.70462 | 1379 | I.S. | 0.70455 | 1380 | APC | 0.7045 |
| 1381 | I.S. | 0.7045 | 1382 | WT1 | 0.70441 | 1383 | LYST | 0.70439 |
| 1384 | I.S. | 0.70433 | 1385 | I.S. | 0.70433 | 1386 | I.S. | 0.7043 |
| 1387 | I.S. | 0.70427 | 1388 | PLCG1 | 0.70423 | 1389 | ZNF276, | 0.7042 |
| | | | | | | | FANCA | |
| 1390 | SBF2 | 0.70418 | 1391 | IRS2 | 0.70418 | 1392 | ASXL1 | 0.70412 |
| 1393 | BRIP1 | 0.70409 | 1394 | I.S. | 0.70409 | 1395 | STAT6 | 0.70408 |
| 1396 | LYST | 0.70406 | 1397 | AKT3 | 0.70404 | 1398 | I.S. | 0.70403 |
| 1399 | BTG1 | 0.704 | 1400 | I.S. | 0.704 | 1401 | FOXP1 | 0.704 |
| 1402 | I.S. | 0.704 | 1403 | BRCA2 | 0.704 | 1404 | HNF1A | 0.704 |
| 1405 | NHP2 | 0.70398 | 1406 | NSD1 | 0.70397 | 1407 | RIT1 | 0.70395 |
| 1408 | I.S. | 0.70395 | 1409 | JAK2 | 0.70394 | 1410 | I.S. | 0.70394 |
| 1411 | I.S. | 0.70392 | 1412 | I.S. | 0.70392 | 1413 | PLCG2 | 0.70391 |
| 1414 | I.S. | 0.70386 | 1415 | I.S. | 0.7038 | 1416 | I.S. | 0.70379 |
| 1417 | LYST | 0.70377 | 1418 | PPM1D | 0.70371 | 1419 | I.S. | 0.7037 |
| 1420 | DDX11 | 0.70368 | 1421 | I.S. | 0.70367 | 1422 | KMT2A | 0.70367 |
| 1423 | I.S. | 0.70365 | 1424 | ERBB2 | 0.70364 | 1425 | SPTA1 | 0.70358 |
| 1426 | FLCN | 0.70358 | 1427 | FANCA | 0.70347 | 1428 | I.S. | 0.70347 |
| 1429 | KMT2B | 0.70347 | 1430 | MLH1 | 0.70346 | 1431 | SETD2 | 0.70346 |
| 1432 | ERBB3 | 0.70343 | 1433 | POLE | 0.7034 | 1434 | I.S. | 0.70338 |
| 1435 | PRKAR1A | 0.70335 | 1436 | I.S. | 0.70332 | 1437 | I.S. | 0.70331 |
| 1438 | MTOR | 0.70329 | 1439 | BRCA2 | 0.70329 | 1440 | FAT1 | 0.70326 |
| 1441 | I.S. | 0.70323 | 1442 | I.S. | 0.70322 | 1443 | EP300 | 0.7032 |
| 1444 | FAT1 | 0.70317 | 1445 | I.S. | 0.70317 | 1446 | INPP4B | 0.70317 |
| 1447 | STAT6 | 0.70317 | 1448 | I.S. | 0.70313 | 1449 | I.S. | 0.7031 |
| 1450 | I.S. | 0.7031 | 1451 | KMT2C | 0.70305 | 1452 | I.S. | 0.70305 |
| 1453 | CDK8 | 0.70305 | 1454 | I.S. | 0.70296 | 1455 | I.S. | 0.70296 |
| 1456 | FAT1 | 0.70296 | 1457 | — | 0.70296 | 1458 | RBBP6 | 0.70296 |
| 1459 | I.S. | 0.70296 | 1460 | MED12 | 0.70294 | 1461 | GID4 | 0.70293 |
| 1462 | TERF2IP | 0.70293 | 1463 | TOP2A | 0.7029 | 1464 | BARD1 | 0.70288 |
| 1465 | I.S. | 0.70288 | 1466 | KDM5A | 0.70287 | 1467 | I.S. | 0.70281 |
| 1468 | BRCA1 | 0.70276 | 1469 | ZNF217 | 0.70276 | 1470 | ALK | 0.70272 |
| 1471 | I.S. | 0.70272 | 1472 | I.S. | 0.70272 | 1473 | I.S. | 0.7027 |
| 1474 | I.S. | 0.70264 | 1475 | I.S. | 0.7026 | 1476 | CCND3 | 0.7026 |
| 1477 | I.S. | 0.7026 | 1478 | I.S. | 0.70253 | 1479 | RUNX1T1 | 0.70252 |
| 1480 | I.S. | 0.7025 | 1481 | I.S. | 0.7025 | 1482 | I.S. | 0.7025 |
| 1483 | I.S. | 0.70249 | 1484 | SBF2 | 0.7024 | 1485 | KDM5A | 0.70239 |
| 1486 | — | 0.70238 | 1487 | FLT1 | 0.70236 | 1488 | I.S. | 0.70234 |
| 1489 | TSC1 | 0.70233 | 1490 | TOP1 | 0.70232 | 1491 | I.S. | 0.70231 |
| 1492 | ERBB2 | 0.70231 | 1493 | I.S. | 0.70228 | 1494 | ATR | 0.70227 |
| 1495 | SLIT2 | 0.70227 | 1496 | FGF14 | 0.70227 | 1497 | FGFR2 | 0.70222 |
| 1498 | HAX1 | 0.70222 | 1499 | PSTPIP1 | 0.70222 | 1500 | TGFBR2 | 0.70219 |
| 1501 | I.S. | 0.70208 | 1502 | NOTCH3 | 0.70203 | 1503 | CARD11 | 0.70198 |
| 1504 | I.S. | 0.70193 | 1505 | JAK2 | 0.70189 | 1506 | I.S. | 0.70186 |
| 1507 | I.S. | 0.70186 | 1508 | I.S. | 0.70186 | 1509 | I.S. | 0.70186 |
| 1510 | MEFV | 0.70186 | 1511 | FOXP1 | 0.70183 | 1512 | FLT4 | 0.70181 |
| 1513 | MAP2K4 | 0.70181 | 1514 | LZTR1 | 0.70178 | 1515 | NOTCH2 | 0.70174 |
| 1516 | NF1 | 0.70174 | 1517 | I.S. | 0.70171 | 1518 | I.S. | 0.70165 |
| 1519 | SMO | 0.70165 | 1520 | I.S. | 0.70165 | 1521 | I.S. | 0.70162 |
| 1522 | BRCA2 | 0.70162 | 1523 | I.S. | 0.7016 | 1524 | PSTPIP1 | 0.70159 |
| 1525 | I.S. | 0.70154 | 1526 | PIK3C2B | 0.70148 | 1527 | FAT1 | 0.70148 |
| 1528 | I.S. | 0.70146 | 1529 | I.S. | 0.70142 | 1530 | PLCG1 | 0.70141 |
| 1531 | I.S. | 0.70141 | 1532 | MAP3K14 | 0.7014 | 1533 | IGF1R | 0.70137 |
| 1534 | ADA | 0.70136 | 1535 | I.S. | 0.70135 | 1536 | BRCA2 | 0.70133 |
| 1537 | I.S. | 0.70124 | 1538 | I.S. | 0.70121 | 1539 | CCNE1 | 0.70115 |
| 1540 | CCNE1 | 0.70115 | 1541 | I.S. | 0.70113 | 1542 | I.S. | 0.70107 |
| 1543 | STAT4 | 0.70104 | 1544 | KMT2A | 0.70094 | 1545 | FAT1 | 0.70091 |
| 1546 | NSD1 | 0.70091 | 1547 | I.S. | 0.70091 | 1548 | I.S. | 0.70088 |
| 1549 | KAT6A | 0.70088 | 1550 | ARID1A | 0.70085 | 1551 | I.S. | 0.70085 |
| 1552 | I.S. | 0.70083 | 1553 | SMO | 0.70083 | 1554 | I.S. | 0.70083 |
| 1555 | POLE | 0.70079 | 1556 | KMT2A | 0.70071 | 1557 | SMAD2 | 0.70071 |
| 1558 | LPIN2 | 0.70071 | 1559 | ADGRA2 | 0.70068 | 1560 | RAD21 | 0.70065 |
| 1561 | BRCA1 | 0.70061 | 1562 | I.S. | 0.70056 | 1563 | I.S. | 0.70053 |
| 1564 | I.S. | 0.7005 | 1565 | ATM | 0.70049 | 1566 | BRCA2 | 0.70049 |
| 1567 | I.S. | 0.70047 | 1568 | SETBP1 | 0.70047 | 1569 | SPTA1 | 0.70044 |
| 1570 | SMO | 0.70043 | 1571 | KEL | 0.70035 | 1572 | I.S. | 0.70026 |
| 1573 | I.S. | 0.70026 | 1574 | I.S. | 0.70026 | 1575 | I.S. | 0.70026 |
| 1576 | I.S. | 0.70023 | 1577 | I.S. | 0.70022 | 1578 | I.S. | 0.70017 |
| 1579 | IGF1R | 0.70017 | 1580 | AURKB | 0.70017 | 1581 | I.S. | 0.70015 |
| 1582 | POLE | 0.70006 | 1583 | SETBP1 | 0.70005 | 1584 | I.S. | 0.70003 |
| 1585 | KMT2B | 0.70003 | 1586 | FH | 0.7 | 1587 | ASXL1 | 0.69997 |
| 1588 | BRCA1 | 0.69996 | 1589 | PRKDC | 0.69993 | 1590 | POLE | 0.69993 |
| 1591 | I.S. | 0.6999 | 1592 | I.S. | 0.69984 | 1593 | I.S. | 0.69979 |
| 1594 | I.S. | 0.69977 | 1595 | GABRA6 | 0.69975 | 1596 | I.S. | 0.69973 |
| 1597 | I.S. | 0.69973 | 1598 | KMT2A | 0.69972 | 1599 | I.S. | 0.69967 |
| 1600 | I.S. | 0.69965 | 1601 | I.S. | 0.69965 | 1602 | CUX1 | 0.6996 |
| 1603 | I.S. | 0.6996 | 1604 | I.S. | 0.69957 | 1605 | KMT2D | 0.69955 |
| 1606 | I.S. | 0.69955 | 1607 | RNF43 | 0.69949 | 1608 | I.S. | 0.69948 |
| 1609 | IRF2 | 0.69948 | 1610 | I.S. | 0.69948 | 1611 | EZH2 | 0.69946 |
| 1612 | RUNX1T1 | 0.69943 | 1613 | I.S. | 0.6994 | 1614 | STAT6 | 0.69939 |
| 1615 | I.S. | 0.6993 | 1616 | GRIN2A | 0.69928 | 1617 | MUTYH | 0.69928 |
| 1618 | RAD51B | 0.69926 | 1619 | RNF43 | 0.69919 | 1620 | SETBP1 | 0.69919 |
| 1621 | FANCA | 0.69918 | 1622 | EMSY | 0.69916 | 1623 | BRCA2 | 0.69915 |
| 1624 | I.S. | 0.69913 | 1625 | I.S. | 0.6991 | 1626 | I.S. | 0.6991 |
| 1627 | ERBB2 | 0.6991 | 1628 | I.S. | 0.6991 | 1629 | MRE11 | 0.69908 |
| 1630 | LZTR1 | 0.69907 | 1631 | I.S. | 0.69902 | 1632 | I.S. | 0.69889 |
| 1633 | EGLN1 | 0.6988 | 1634 | ATM | 0.69878 | 1635 | I.S. | 0.69877 |
| 1636 | I.S. | 0.69877 | 1637 | RTEL1 | 0.69877 | 1638 | SDHC | 0.69875 |
| 1639 | NSD1 | 0.69874 | 1640 | STAT3 | 0.69871 | 1641 | RUNX1T1 | 0.69866 |
| 1642 | RAD51B | 0.69866 | 1643 | I.S. | 0.69866 | 1644 | CTC1 | 0.69862 |
| 1645 | SETD2 | 0.69861 | 1646 | KMT2B | 0.69857 | 1647 | EPAS1 | 0.69856 |
| 1648 | I.S. | 0.69856 | 1649 | FGFR2 | 0.69853 | 1650 | LMO1 | 0.69853 |
| 1651 | I.S. | 0.69847 | 1652 | I.S. | 0.69845 | 1653 | I.S. | 0.69845 |
| 1654 | FAT1 | 0.69842 | 1655 | I.S. | 0.69839 | 1656 | I.S. | 0.69836 |
| 1657 | PREX2 | 0.69836 | 1658 | I.S. | 0.69834 | 1659 | LYST | 0.69827 |
| 1660 | SRC | 0.69827 | 1661 | SETD2 | 0.69825 | 1662 | I.S. | 0.69812 |
| 1663 | I.S. | 0.69812 | 1664 | I.S. | 0.69812 | 1665 | NKX2-1 | 0.69812 |
| 1666 | FANCC | 0.69812 | 1667 | I.S. | 0.69803 | 1668 | IKBKE | 0.69798 |
| 1669 | KLC1, | 0.69795 | 1670 | I.S. | 0.69792 | 1671 | FANCE | 0.69788 |
| | XRCC3 | | | | | | | |
| 1672 | I.S. | 0.69788 | 1673 | I.S. | 0.69788 | 1674 | I.S. | 0.69786 |
| 1675 | IKBKE | 0.69785 | 1676 | I.S. | 0.69782 | 1677 | I.S. | 0.69782 |
| 1678 | I.S. | 0.69779 | 1679 | FANCI | 0.69779 | 1680 | I.S. | 0.69771 |
| 1681 | I.S. | 0.6977 | 1682 | RAD21 | 0.6977 | 1683 | I.S. | 0.69765 |
| 1684 | I.S. | 0.69765 | 1685 | RTEL1 | 0.69762 | 1686 | ESR1 | 0.69762 |
| 1687 | NOTCH2 | 0.69759 | 1688 | I.S. | 0.69759 | 1689 | NHP2 | 0.69756 |
| 1690 | I.S. | 0.69755 | 1691 | I.S. | 0.69749 | 1692 | MAP3K14 | 0.69747 |
| 1693 | I.S. | 0.69747 | 1694 | CIC | 0.69746 | 1695 | I.S. | 0.69743 |
| 1696 | I.S. | 0.69738 | 1697 | I.S. | 0.69737 | 1698 | I.S. | 0.69737 |
| 1699 | TET2 | 0.69737 | 1700 | KIF23 | 0.69737 | 1701 | I.S. | 0.69733 |
| 1702 | I.S. | 0.69729 | 1703 | RUNX1T1 | 0.69729 | 1704 | SMAD4 | 0.69726 |
| 1705 | I.S. | 0.69726 | 1706 | ROS1 | 0.69726 | 1707 | I.S. | 0.69724 |
| 1708 | I.S. | 0.69716 | 1709 | I.S. | 0.69716 | 1710 | FLI1 | 0.69716 |
| 1711 | FGFR1 | 0.69716 | 1712 | RAD51B | 0.69712 | 1713 | PIK3C2B | 0.69711 |
| 1714 | I.S. | 0.69708 | 1715 | I.S. | 0.69706 | 1716 | SPEN | 0.69705 |
| 1717 | I.S. | 0.69705 | 1718 | PIK3CA | 0.69705 | 1719 | I.S. | 0.69705 |
| 1720 | I.S. | 0.69705 | 1721 | I.S. | 0.69702 | 1722 | QKI | 0.697 |
| 1723 | PLCG1 | 0.697 | 1724 | I.S. | 0.69696 | 1725 | I.S. | 0.69696 |
| 1726 | RARA | 0.69696 | 1727 | ARID1B | 0.69695 | 1728 | I.S. | 0.69694 |
| 1729 | LRP1B | 0.69693 | 1730 | PMS2 | 0.69687 | 1731 | CCND3 | 0.69685 |
| 1732 | SPTA1 | 0.69684 | 1733 | I.S. | 0.69684 | 1734 | I.S. | 0.69682 |
| 1735 | PABPN1 | 0.69682 | 1736 | I.S. | 0.69679 | 1737 | I.S. | 0.69673 |
| 1738 | FAT1 | 0.69666 | 1739 | ID3 | 0.69664 | 1740 | I.S. | 0.69664 |
| 1741 | FRS2 | 0.69664 | 1742 | GNA13 | 0.69664 | 1743 | I.S. | 0.69661 |
| 1744 | I.S. | 0.69661 | 1745 | I.S. | 0.6966 | 1746 | RET | 0.69657 |
| 1747 | I.S. | 0.69655 | 1748 | CRKL | 0.69655 | 1749 | KMT2C | 0.69651 |
| 1750 | DAXX | 0.69646 | 1751 | SBF2 | 0.69643 | 1752 | TP53 | 0.69639 |
| 1753 | I.S. | 0.69639 | 1754 | I.S. | 0.69637 | 1755 | BCL6 | 0.69637 |
| 1756 | KMT2C | 0.69637 | 1757 | KMT2D | 0.69636 | 1758 | EZH2 | 0.69634 |
| 1759 | RAD51D | 0.69634 | 1760 | I.S. | 0.69631 | 1761 | I.S. | 0.69631 |
| 1762 | I.S. | 0.69629 | 1763 | I.S. | 0.69627 | 1764 | DICER1 | 0.69623 |
| 1765 | PRSS8 | 0.6962 | 1766 | I.S. | 0.69619 | 1767 | ERBB4 | 0.69618 |
| 1768 | CTC1 | 0.69616 | 1769 | CTNNB1 | 0.69614 | 1770 | PRKN | 0.69614 |
| 1771 | GNAS | 0.6961 | 1772 | SDHA | 0.69607 | 1773 | I.S. | 0.69607 |
| 1774 | I.S. | 0.69607 | 1775 | RICTOR | 0.69603 | 1776 | I.S. | 0.69602 |
| 1777 | ERBB2 | 0.69598 | 1778 | PLCG2 | 0.69593 | 1779 | I.S. | 0.6959 |
| 1780 | NOTCH2 | 0.69589 | 1781 | GNAS | 0.69586 | 1782 | KMT2D | 0.69586 |
| 1783 | CBL | 0.69584 | 1784 | I.S. | 0.69581 | 1785 | FAT1 | 0.69581 |
| 1786 | I.S. | 0.69578 | 1787 | DDX41 | 0.69577 | 1788 | FANCM | 0.69577 |
| 1789 | TRAF3 | 0.69575 | 1790 | I.S. | 0.69574 | 1791 | I.S. | 0.69572 |
| 1792 | PRSS8 | 0.69569 | 1793 | SMARCA4 | 0.69566 | 1794 | I.S. | 0.69565 |
| 1795 | RICTOR | 0.69565 | 1796 | ROS1 | 0.69565 | 1797 | I.S. | 0.69563 |
| 1798 | IKBKE | 0.69563 | 1799 | I.S. | 0.6956 | 1800 | SRC | 0.6956 |
| 1801 | I.S. | 0.69558 | 1802 | I.S. | 0.69558 | 1803 | I.S. | 0.69556 |
| 1804 | I.S. | 0.69535 | 1805 | I.S. | 0.69533 | 1806 | I.S. | 0.69527 |
| 1807 | PRDM1 | 0.69527 | 1808 | I.S. | 0.69525 | 1809 | ADGRA2 | 0.69524 |
| 1810 | ATG2B | 0.69524 | 1811 | GRIN2A | 0.69524 | 1812 | LPIN2 | 0.69524 |
| 1813 | RPTOR | 0.69522 | 1814 | I.S. | 0.69522 | 1815 | I.S. | 0.69519 |
| 1816 | I.S. | 0.69517 | 1817 | I.S. | 0.69516 | 1818 | FLI1, SENCR | 0.69516 |
| 1819 | MAP3K14 | 0.69514 | 1820 | CHD4 | 0.69514 | 1821 | IDH2 | 0.69512 |
| 1822 | SPTA1 | 0.69511 | 1823 | BRCA2 | 0.69508 | 1824 | ABL2 | 0.69506 |
| 1825 | I.S. | 0.69503 | 1826 | I.S. | 0.69503 | 1827 | — | 0.69503 |
| 1828 | I.S. | 0.69503 | 1829 | I.S. | 0.69503 | 1830 | I.S. | 0.69503 |
| 1831 | I.S. | 0.69503 | 1832 | I.S. | 0.69503 | 1833 | PTCH1 | 0.69503 |
| 1834 | I.S. | 0.69503 | 1835 | I.S. | 0.69503 | 1836 | PRKCI | 0.69503 |
| 1837 | I.S. | 0.695 | 1838 | CIC | 0.69498 | 1839 | MDM4 | 0.69497 |
| 1840 | PSTPIP1 | 0.69494 | 1841 | I.S. | 0.69491 | 1842 | FLT3 | 0.69491 |
| 1843 | FOXP1 | 0.69487 | 1844 | I.S. | 0.69483 | 1845 | ERBB2 | 0.69483 |
| 1846 | I.S. | 0.69482 | 1847 | I.S. | 0.6948 | 1848 | KMT2C | 0.69476 |
| 1849 | FGFR2 | 0.69474 | 1850 | I.S. | 0.69472 | 1851 | I.S. | 0.6947 |
| 1852 | I.S. | 0.6947 | 1853 | I.S. | 0.6947 | 1854 | GATA3 | 0.69467 |
| 1855 | FLT3 | 0.69462 | 1856 | I.S. | 0.69458 | 1857 | ALK | 0.69458 |
| 1858 | I.S. | 0.69456 | 1859 | I.S. | 0.69453 | 1860 | I.S. | 0.6945 |
| 1861 | I.S. | 0.69449 | 1862 | I.S. | 0.69448 | 1863 | I.S. | 0.69447 |
| 1864 | I.S. | 0.69444 | 1865 | RAD51D | 0.69444 | 1866 | I.S. | 0.69441 |
| 1867 | I.S. | 0.69441 | 1868 | I.S. | 0.69439 | 1869 | FANCA | 0.69438 |
| 1870 | I.S. | 0.69437 | 1871 | I.S. | 0.69437 | 1872 | I.S. | 0.69435 |
| 1873 | BCR | 0.69434 | 1874 | MEN1 | 0.69433 | 1875 | I.S. | 0.69432 |
| 1876 | I.S. | 0.69426 | 1877 | I.S. | 0.69423 | 1878 | I.S. | 0.69422 |
| 1879 | I.S. | 0.6942 | 1880 | FH | 0.69418 | 1881 | GATA2 | 0.69417 |
| 1882 | NSD2 | 0.69416 | 1883 | I.S. | 0.69408 | 1884 | FANCD2 | 0.69405 |
| 1885 | ROS1 | 0.69405 | 1886 | EP300 | 0.69405 | 1887 | NTRK2 | 0.694 |
| 1888 | I.S. | 0.69399 | 1889 | I.S. | 0.69399 | 1890 | PIK3CA | 0.69399 |
| 1891 | JAK2 | 0.69399 | 1892 | IRF2 | 0.69396 | 1893 | EMSY | 0.69396 |
| 1894 | I.S. | 0.69393 | 1895 | I.S. | 0.69384 | 1896 | KMT2C | 0.69369 |
| 1897 | IKBKE | 0.69367 | 1898 | DOT1L | 0.69367 | 1899 | RAD50 | 0.69366 |
| 1900 | GRIN2A | 0.69363 | 1901 | NBN | 0.69363 | 1902 | FLT4 | 0.6936 |
| 1903 | ESR1 | 0.6936 | 1904 | INHBA | 0.69355 | 1905 | I.S. | 0.69352 |
| 1906 | I.S. | 0.69351 | 1907 | HAX1 | 0.69346 | 1908 | NBN | 0.69343 |
| 1909 | I.S. | 0.69341 | 1910 | LYST | 0.6934 | 1911 | CSF1R | 0.69337 |
| 1912 | I.S. | 0.69335 | 1913 | I.S. | 0.69334 | 1914 | TSC1 | 0.69334 |
| 1915 | I.S. | 0.69332 | 1916 | CBL | 0.69325 | 1917 | MAP3K14 | 0.69322 |
| 1918 | I.S. | 0.69319 | 1919 | I.S. | 0.69313 | 1920 | I.S. | 0.69313 |
| 1921 | I.S. | 0.6931 | 1922 | I.S. | 0.6931 | 1923 | IRS2 | 0.69307 |
| 1924 | STAT6 | 0.69304 | 1925 | TOP2A | 0.69302 | 1926 | KMT2C | 0.69295 |
| 1927 | I.S. | 0.69293 | 1928 | HNF1A | 0.69292 | 1929 | I.S. | 0.69289 |
| 1930 | I.S. | 0.69289 | 1931 | IGF1R | 0.69289 | 1932 | I.S. | 0.69289 |
| 1933 | I.S. | 0.69289 | 1934 | IGF1R | 0.69289 | 1935 | SLIT2 | 0.69286 |
| 1936 | I.S. | 0.69286 | 1937 | GLI1 | 0.69275 | 1938 | TERF2 | 0.69275 |
| 1939 | PLCG1 | 0.69275 | 1940 | I.S. | 0.69273 | 1941 | CTC1 | 0.69272 |
| 1942 | TOP2A | 0.69269 | 1943 | AXL | 0.69269 | 1944 | I.S. | 0.69267 |
| 1945 | FGFR4 | 0.69258 | 1946 | DOT1L | 0.69256 | 1947 | I.S. | 0.69256 |
| 1948 | I.S. | 0.69256 | 1949 | SPTA1 | 0.69254 | 1950 | I.S. | 0.69254 |
| 1951 | KMT2B | 0.69254 | 1952 | FLI1 | 0.69252 | 1953 | PRDM1 | 0.69251 |
| 1954 | I.S. | 0.69248 | 1955 | I.S. | 0.69245 | 1956 | I.S. | 0.69243 |
| 1957 | ABL2 | 0.69242 | 1958 | I.S. | 0.69242 | 1959 | I.S. | 0.69242 |
| 1960 | BCR | 0.69239 | 1961 | PIK3C2B | 0.69237 | 1962 | FLT1 | 0.69235 |
| 1963 | ATG2B | 0.69227 | 1964 | DNMT3A | 0.69227 | 1965 | NSD1 | 0.69226 |
| 1966 | I.S. | 0.69225 | 1967 | ATM | 0.69224 | 1968 | IRS2 | 0.69224 |
| 1969 | SETBP1 | 0.69224 | 1970 | POLR2F, | 0.69223 | 1971 | I.S. | 0.69221 |
| | | | | SOX10 | | | | |
| 1972 | TRAF3 | 0.6922 | 1973 | KMT2D | 0.69218 | 1974 | LYST | 0.69212 |
| 1975 | I.S. | 0.69212 | 1976 | I.S. | 0.69206 | 1977 | NLRP3 | 0.69204 |
| 1978 | MDM2 | 0.69203 | 1979 | PTCH1 | 0.69201 | 1980 | MDM4 | 0.69198 |
| 1981 | I.S. | 0.69196 | 1982 | I.S. | 0.69194 | 1983 | JAK3 | 0.69185 |
| 1984 | CDK12 | 0.69185 | 1985 | NOTCH3 | 0.69185 | 1986 | I.S. | 0.69182 |
| 1987 | I.S. | 0.6918 | 1988 | FOXP1 | 0.69179 | 1989 | I.S. | 0.69177 |
| 1990 | I.S. | 0.69171 | 1991 | LYST | 0.6917 | 1992 | ROS1 | 0.69165 |
| 1993 | I.S. | 0.69164 | 1994 | I.S. | 0.69163 | 1995 | SOX2-OT, | 0.69162 |
| | | | | | | | SOX2 | |
| 1996 | CHEK1 | 0.69161 | 1997 | I.S. | 0.69156 | 1998 | I.S. | 0.69153 |
| 1999 | NUP93 | 0.6915 | 2000 | I.S. | 0.69149 | 2001 | I.S. | 0.69149 |
| 2002 | ERBB2 | 0.69146 | 2003 | I.S. | 0.69144 | 2004 | ACD | 0.69144 |
| 2005 | KMT2C | 0.69143 | 2006 | CXCR4 | 0.69141 | 2007 | SMC3 | 0.69141 |
| 2008 | I.S. | 0.69136 | 2009 | CTC1 | 0.69136 | 2010 | MYC | 0.69135 |
| 2011 | I.S. | 0.69135 | 2012 | ZNF217 | 0.69135 | 2013 | FGF14 | 0.69121 |
| 2014 | DNMT3A | 0.6912 | 2015 | MAP3K14 | 0.69117 | 2016 | I.S. | 0.69117 |
| 2017 | I.S. | 0.69117 | 2018 | I.S. | 0.69117 | 2019 | I.S. | 0.69113 |
| 2020 | I.S. | 0.69113 | 2021 | I.S. | 0.69111 | 2022 | SLIT2 | 0.69108 |
| 2023 | I.S. | 0.69108 | 2024 | I.S. | 0.69106 | 2025 | RET | 0.69103 |
| 2026 | ZNF217 | 0.69103 | 2027 | NTRK3 | 0.69099 | 2028 | BRCA2 | 0.69095 |
| 2029 | I.S. | 0.69095 | 2030 | KMT2C | 0.69095 | 2031 | RICTOR | 0.69094 |
| 2032 | FANCM | 0.69094 | 2033 | CSF1R | 0.69087 | 2034 | BRCA1 | 0.69087 |
| 2035 | I.S. | 0.69087 | 2036 | SDHA | 0.69087 | 2037 | TET2 | 0.69084 |
| 2038 | PTCH1 | 0.69084 | 2039 | I.S. | 0.69081 | 2040 | RTEL1 | 0.69079 |
| 2041 | DICER1 | 0.69076 | 2042 | I.S. | 0.69075 | 2043 | I.S. | 0.69073 |
| 2044 | I.S. | 0.69069 | 2045 | SDHA | 0.69066 | 2046 | POLE | 0.69066 |
| 2047 | CCND3 | 0.69058 | 2048 | TGFBR2 | 0.69058 | 2049 | AR | 0.69055 |
| 2050 | BCL2L1 | 0.69046 | 2051 | KMT2C | 0.69045 | 2052 | SLIT2 | 0.69045 |
| 2053 | I.S. | 0.69043 | 2054 | TBX3 | 0.6904 | 2055 | PIK3R2 | 0.6904 |
| 2056 | CDK4 | 0.69037 | 2057 | I.S. | 0.69037 | 2058 | SPTA1 | 0.69037 |
| 2059 | I.S. | 0.69035 | 2060 | PIK3CA | 0.69034 | 2061 | CDH1 | 0.69034 |
| 2062 | ASXL1 | 0.69034 | 2063 | ATM | 0.69033 | 2064 | I.S. | 0.69029 |
| 2065 | CHEK2 | 0.69025 | 2066 | I.S. | 0.69025 | 2067 | ASXL1 | 0.69018 |
| 2068 | I.S. | 0.69016 | 2069 | I.S. | 0.69013 | 2070 | I.S. | 0.69013 |
| 2071 | LYN | 0.69013 | 2072 | I.S. | 0.6901 | 2073 | I.S. | 0.6901 |
| 2074 | FANCL | 0.69004 | 2075 | I.S. | 0.69004 | 2076 | I.S. | 0.69004 |
| 2077 | I.S. | 0.69004 | 2078 | I.S. | 0.69002 | 2079 | IL7R | 0.68998 |
| 2080 | I.S. | 0.68998 | 2081 | ASXL1 | 0.68995 | 2082 | I.S. | 0.68993 |
| 2083 | ETV6 | 0.68993 | 2084 | FANCI | 0.68989 | 2085 | I.S. | 0.68972 |
| 2086 | I.S. | 0.68972 | 2087 | I.S. | 0.68972 | 2088 | I.S. | 0.68969 |
| 2089 | I.S. | 0.68968 | 2090 | I.S. | 0.6896 | 2091 | CHD2 | 0.68956 |
| 2092 | MYC | 0.68954 | 2093 | ERBB4 | 0.68949 | 2094 | SLIT2 | 0.68948 |
| 2095 | I.S. | 0.68948 | 2096 | I.S. | 0.68947 | 2097 | I.S. | 0.68947 |
| 2098 | I.S. | 0.68947 | 2099 | I.S. | 0.68947 | 2100 | I.S. | 0.68947 |
| 2101 | I.S. | 0.68939 | 2102 | I.S. | 0.68939 | 2103 | CSF1R | 0.68939 |
| 2104 | I.S. | 0.68936 | 2105 | MET | 0.68936 | 2106 | I.S. | 0.68936 |
| 2107 | HNF1A | 0.68933 | 2108 | PIK3R2 | 0.68933 | 2109 | I.S. | 0.68932 |
| 2110 | IKBKE | 0.6893 | 2111 | SMARCA4 | 0.6893 | 2112 | I.S. | 0.68928 |
| 2113 | I.S. | 0.68927 | 2114 | KMT2D | 0.68927 | 2115 | DAXX | 0.68924 |
| 2116 | I.S. | 0.68924 | 2117 | CIC | 0.68919 | 2118 | I.S. | 0.68915 |
| 2119 | NOTCH3 | 0.68915 | 2120 | I.S. | 0.68915 | 2121 | KMT2C | 0.68912 |
| 2122 | I.S. | 0.68909 | 2123 | ATG2B | 0.68909 | 2124 | AXL | 0.68904 |
| 2125 | SLIT2 | 0.689 | 2126 | ZBTB2 | 0.68897 | 2127 | PRKCI | 0.68897 |
| 2128 | ERBB3 | 0.68897 | 2129 | SMAD9 | 0.68894 | 2130 | I.S. | 0.68894 |
| 2131 | I.S. | 0.68894 | 2132 | I.S. | 0.68894 | 2133 | I.S. | 0.68892 |
| 2134 | PIK3R1 | 0.68889 | 2135 | CREBBP | 0.68885 | 2136 | NF1 | 0.68884 |
| 2137 | ZNF217 | 0.68877 | 2138 | I.S. | 0.68873 | 2139 | I.S. | 0.68864 |
| 2140 | RBBP6 | 0.68864 | 2141 | PLCG1 | 0.68864 | 2142 | NSD1 | 0.68856 |
| 2143 | NSD1 | 0.68855 | 2144 | PRKCI | 0.68853 | 2145 | I.S. | 0.6885 |
| 2146 | BRCA2 | 0.68849 | 2147 | JAK2 | 0.68849 | 2148 | CHD2 | 0.68849 |
| 2149 | GLI1 | 0.68849 | 2150 | AKT2 | 0.68847 | 2151 | I.S. | 0.68843 |
| 2152 | PIK3C2B | 0.68843 | 2153 | I.S. | 0.68839 | 2154 | I.S. | 0.68835 |
| 2155 | I.S. | 0.68832 | 2156 | I.S. | 0.68832 | 2157 | I.S. | 0.6883 |
| 2158 | I.S. | 0.68827 | 2159 | APC | 0.68826 | 2160 | FGFR3 | 0.68823 |
| 2161 | I.S. | 0.68823 | 2162 | I.S. | 0.68821 | 2163 | NFE2L2 | 0.68816 |
| 2164 | CXCR4 | 0.68814 | 2165 | I.S. | 0.68812 | 2166 | I.S. | 0.68811 |
| 2167 | CTCF | 0.68811 | 2168 | I.S. | 0.68811 | 2169 | STAT4 | 0.68809 |
| 2170 | MEFV | 0.68805 | 2171 | ATR | 0.68802 | 2172 | I.S. | 0.688 |
| 2173 | EGFR | 0.688 | 2174 | SLIT2 | 0.68799 | 2175 | KMT2C | 0.68799 |
| 2176 | I.S. | 0.68796 | 2177 | I.S. | 0.68796 | 2178 | ADGRA2 | 0.68796 |
| 2179 | I.S. | 0.68796 | 2180 | I.S. | 0.68795 | 2181 | 1I.S. | 0.68793 |
| 2182 | I.S. | 0.68792 | 2183 | ALK | 0.68791 | 2184 | I.S. | 0.68788 |
| 2185 | KMT2B | 0.68788 | 2186 | IGF1R | 0.68788 | 2187 | ABL1 | 0.68786 |
| 2188 | I.S. | 0.68782 | 2189 | CDC73 | 0.68782 | 2190 | ATR | 0.68778 |
| 2191 | I.S. | 0.6877 | 2192 | I.S. | 0.6877 | 2193 | TOP2A | 0.6877 |
| 2194 | CCN6 | 0.68766 | 2195 | I.S. | 0.68766 | 2196 | I.S. | 0.68761 |
| 2197 | I.S. | 0.68761 | 2198 | I.S. | 0.6876 | 2199 | I.S. | 0.68755 |
| 2200 | NF1 | 0.68754 | 2201 | I.S. | 0.68753 | 2202 | I.S. | 0.68751 |
| 2203 | KMT2C | 0.68749 | 2204 | I.S. | 0.68745 | 2205 | SRC | 0.68742 |
| 2206 | ERBB4 | 0.68742 | 2207 | PTCH1 | 0.68739 | 2208 | IDH1 | 0.68737 |
| 2209 | AXL | 0.68737 | 2210 | PLCG1 | 0.68737 | 2211 | I.S. | 0.68735 |
| 2212 | I.S. | 0.68734 | 2213 | I.S. | 0.68731 | 2214 | KMT2A | 0.6873 |
| 2215 | PAX5 | 0.6873 | 2216 | LYST | 0.68728 | 2217 | LZTR1 | 0.68725 |
| 2218 | ABL2 | 0.68725 | 2219 | — | 0.68722 | 2220 | FGFR2 | 0.68722 |
| 2221 | I.S. | 0.68722 | 2222 | DDR2 | 0.6872 | 2223 | I.S. | 0.68719 |
| 2224 | ROS1 | 0.68716 | 2225 | XRCC3 | 0.6871 | 2226 | I.S. | 0.68709 |
| 2227 | I.S. | 0.68709 | 2228 | I.S. | 0.68708 | 2229 | KMT2C | 0.68707 |
| 2230 | NSD1 | 0.68706 | 2231 | I.S. | 0.68701 | 2232 | I.S. | 0.68697 |
| 2233 | LRP1B | 0.68693 | 2234 | CCND3 | 0.68692 | 2235 | I.S. | 0.68689 |
| 2236 | EPHA7 | 0.68689 | 2237 | I.S. | 0.68687 | 2238 | ABL1 | 0.68683 |
| 2239 | I.S. | 0.68683 | 2240 | I.S. | 0.6868 | 2241 | I.S. | 0.68679 |
| 2242 | I.S. | 0.68679 | 2243 | FANCI, | 0.68677 | 2244 | I.S. | 0.68671 |
| | | | | POLG | | | | |
| 2245 | RAB27A | 0.68668 | 2246 | PTPN11 | 0.68667 | 2247 | PRSS1 | 0.68666 |
| 2248 | I.S. | 0.68663 | 2249 | CARMIL2, | 0.68661 | 2250 | I.S. | 0.6866 |
| | | | | ACD | | | | |
| 2251 | I.S. | 0.6866 | 2252 | KMT2C | 0.68659 | 2253 | LRP1B | 0.68656 |
| 2254 | FLT1 | 0.68654 | 2255 | I.S. | 0.68654 | 2256 | I.S. | 0.68654 |
| 2257 | PLCG2 | 0.68654 | 2258 | I.S. | 0.68652 | 2259 | RHEB | 0.68651 |
| 2260 | I.S. | 0.68651 | 2261 | I.S. | 0.68651 | 2262 | I.S. | 0.68651 |
| 2263 | I.S. | 0.68649 | 2264 | ERBB4 | 0.68647 | 2265 | TOP2A | 0.68647 |
| 2266 | FH | 0.68645 | 2267 | ALK | 0.68642 | 2268 | I.S. | 0.68641 |
| 2269 | I.S. | 0.68638 | 2270 | I.S. | 0.68638 | 2271 | I.S. | 0.68636 |
| 2272 | I.S. | 0.68632 | 2273 | I.S. | 0.68631 | 2274 | I.S. | 0.6863 |
| 2275 | I.S. | 0.6863 | 2276 | RANBP2 | 0.68627 | 2277 | I.S. | 0.68626 |
| 2278 | I.S. | 0.68626 | 2279 | I.S. | 0.68624 | 2280 | I.S. | 0.68624 |
| 2281 | LYST | 0.68618 | 2282 | I.S. | 0.68618 | 2283 | TBX3 | 0.68618 |
| 2284 | RPTOR | 0.68618 | 2285 | I.S. | 0.68616 | 2286 | I.S. | 0.68615 |
| 2287 | I.S. | 0.68611 | 2288 | I.S. | 0.6861 | 2289 | SMO | 0.68606 |
| 2290 | CTC1 | 0.68606 | 2291 | NF1 | 0.68605 | 2292 | MTOR | 0.68603 |
| 2293 | FOXP1 | 0.68603 | 2294 | I.S. | 0.68602 | 2295 | I.S. | 0.68594 |
| 2296 | I.S. | 0.68594 | 2297 | I.S. | 0.68589 | 2298 | ATM | 0.68588 |
| 2299 | I.S. | 0.68586 | 2300 | I.S. | 0.68586 | 2301 | BRCA1 | 0.68586 |
| 2302 | HAX1 | 0.68583 | 2303 | IKBKE | 0.68583 | 2304 | I.S. | 0.68582 |
| 2305 | I.S. | 0.6858 | 2306 | RAD54L | 0.6858 | 2307 | LZTR1 | 0.68577 |
| 2308 | CCND3 | 0.68565 | 2309 | I.S. | 0.68558 | 2310 | I.S. | 0.68556 |
| 2311 | FGFR1 | 0.68555 | 2312 | PPM1D | 0.68553 | 2313 | MLH1 | 0.68553 |
| 2314 | I.S. | 0.68553 | 2315 | FANCA | 0.68553 | 2316 | I.S. | 0.6855 |
| 2317 | BRAF | 0.68544 | 2318 | I.S. | 0.68544 | 2319 | ERBB4 | 0.68544 |
| 2320 | I.S. | 0.68544 | 2321 | NTRK3 | 0.68544 | 2322 | I.S. | 0.68542 |
| 2323 | I.S. | 0.6854 | 2324 | I.S. | 0.68539 | 2325 | I.S. | 0.68535 |
| 2326 | PIK3CB | 0.68531 | 2327 | I.S. | 0.68531 | 2328 | I.S. | 0.68526 |
| 2329 | POLE | 0.68525 | 2330 | LYST | 0.6852 | 2331 | I.S. | 0.6852 |
| 2332 | STAT6 | 0.6852 | 2333 | PTPN11 | 0.6852 | 2334 | ATM | 0.68514 |
| 2335 | ABL1 | 0.68512 | 2336 | CHEK1 | 0.68511 | 2337 | PSTPIP1 | 0.68508 |
| 2338 | I.S. | 0.68508 | 2339 | ALK | 0.68508 | 2340 | PAK3 | 0.68508 |
| 2341 | I.S. | 0.68507 | 2342 | AURKC | 0.68499 | 2343 | FLT1 | 0.68498 |
| 2344 | KIF23 | 0.68496 | 2345 | I.S. | 0.68494 | 2346 | I.S. | 0.68493 |
| 2347 | I.S. | 0.68491 | 2348 | I.S. | 0.68487 | 2349 | I.S. | 0.68487 |
| 2350 | I.S. | 0.68487 | 2351 | CTCF | 0.68485 | 2352 | I.S. | 0.68485 |
| 2353 | I.S. | 0.68483 | 2354 | AXL | 0.68482 | 2355 | FLT1 | 0.68481 |
| 2356 | LZTR1 | 0.68481 | 2357 | I.S. | 0.68479 | 2358 | I.S. | 0.68478 |
| 2359 | I.S. | 0.68477 | 2360 | RB1 | 0.6847 | 2361 | I.S. | 0.68469 |
| 2362 | LYST | 0.68466 | 2363 | I.S. | 0.68463 | 2364 | TET2 | 0.68463 |
| 2365 | MAP3K1 | 0.68463 | 2366 | — | 0.6846 | 2367 | NSD2 | 0.68457 |
| 2368 | NTRK3 | 0.68457 | 2369 | KMT2D | 0.68455 | 2370 | I.S. | 0.68454 |
| 2371 | APC | 0.68454 | 2372 | DNM2 | 0.68452 | 2373 | I.S. | 0.68447 |
| 2374 | I.S. | 0.68447 | 2375 | MAP3K14 | 0.68446 | 2376 | I.S. | 0.68443 |
| 2377 | KMT2A | 0.68442 | 2378 | I.S. | 0.6844 | 2379 | I.S. | 0.6844 |
| 2380 | TP53 | 0.68437 | 2381 | POLR2F, | 0.68437 | 2382 | SPTA1 | 0.68436 |
| | | | | SOX10 | | | | |
| 2383 | IRS2 | 0.6843 | 2384 | CEBPA | 0.68429 | 2385 | NTRK3 | 0.68428 |
| 2386 | PLCG1 | 0.68428 | 2387 | I.S. | 0.68424 | 2388 | GSK3B | 0.68416 |
| 2389 | INPP4B | 0.68416 | 2390 | I.S. | 0.68414 | 2391 | CBLB | 0.68404 |
| 2392 | CSF1R | 0.684 | 2393 | BRCA1 | 0.68395 | 2394 | I.S. | 0.68394 |
| 2395 | I.S. | 0.68392 | 2396 | I.S. | 0.68392 | 2397 | I.S. | 0.68392 |
| 2398 | I.S. | 0.68392 | 2399 | I.S. | 0.68392 | 2400 | I.S. | 0.68392 |
| 2401 | I.S. | 0.68392 | 2402 | BLM | 0.68389 | 2403 | FGFR1 | 0.68389 |
| 2404 | I.S. | 0.68383 | 2405 | I.S. | 0.68381 | 2406 | I.S. | 0.68381 |
| 2407 | I.S. | 0.6838 | 2408 | FLT3 | 0.68378 | 2409 | GLI1 | 0.68375 |
| 2410 | GLI1 | 0.68375 | 2411 | I.S. | 0.68373 | 2412 | TERC | 0.68372 |
| 2413 | I.S. | 0.6837 | 2414 | I.S. | 0.68369 | 2415 | JAK3 | 0.68369 |
| 2416 | I.S. | 0.68368 | 2417 | GLI2 | 0.68368 | 2418 | I.S. | 0.68361 |
| 2419 | CDKN1A | 0.6836 | 2420 | ROS1 | 0.6836 | 2421 | I.S. | 0.68359 |
| 2422 | RICTOR | 0.68359 | 2423 | I.S. | 0.68359 | 2424 | EGFR | 0.68359 |
| 2425 | MTOR | 0.68356 | 2426 | KMT2D | 0.68351 | 2427 | I.S. | 0.68351 |
| 2428 | I.S. | 0.6835 | 2429 | VEGFA | 0.6835 | 2430 | GRM3 | 0.68347 |
| 2431 | I.S. | 0.68347 | 2432 | ALK | 0.68344 | 2433 | I.S. | 0.68343 |
| 2434 | ACD | 0.68342 | 2435 | FH | 0.68339 | 2436 | I.S. | 0.68339 |
| 2437 | I.S. | 0.68338 | 2438 | I.S. | 0.68337 | 2439 | LZTR1 | 0.68331 |
| 2440 | I.S. | 0.68326 | 2441 | I.S. | 0.68322 | 2442 | I.S. | 0.68322 |
| 2443 | RANBP2 | 0.68318 | 2444 | FGF14 | 0.68317 | 2445 | I.S. | 0.68312 |
| 2446 | ROS1 | 0.68311 | 2447 | I.S. | 0.6831 | 2448 | EXO1 | 0.68309 |
| 2449 | NSD1 | 0.68309 | 2450 | ACVR1B | 0.68309 | 2451 | POLE | 0.68309 |
| 2452 | I.S. | 0.68306 | 2453 | I.S. | 0.68306 | 2454 | I.S. | 0.68301 |
| 2455 | CDH1 | 0.683 | 2456 | CTC1 | 0.68297 | 2457 | SBF2 | 0.68297 |
| 2458 | I.S. | 0.68296 | 2459 | I.S. | 0.68295 | 2460 | TSC2 | 0.68295 |
| 2461 | ARID1B | 0.68291 | 2462 | I.S. | 0.6829 | 2463 | I.S. | 0.68288 |
| 2464 | ATM | 0.68284 | 2465 | PLCG1 | 0.6828 | 2466 | I.S. | 0.68276 |
| 2467 | RICTOR | 0.68276 | 2468 | I.S. | 0.68273 | 2469 | SMAD4 | 0.68273 |
| 2470 | PAX5 | 0.6827 | 2471 | KMT2C | 0.68269 | 2472 | I.S. | 0.68268 |
| 2473 | — | 0.68268 | 2474 | I.S. | 0.68268 | 2475 | I.S. | 0.68268 |
| 2476 | GLI2 | 0.68268 | 2477 | I.S. | 0.68266 | 2478 | I.S. | 0.68266 |
| 2479 | FAT1 | 0.68264 | 2480 | KDM5A | 0.68263 | 2481 | I.S. | 0.68257 |
| 2482 | CDKN2A | 0.68256 | 2483 | I.S. | 0.68255 | 2484 | I.S. | 0.68255 |
| 2485 | I.S. | 0.68249 | 2486 | NF2 | 0.68249 | 2487 | I.S. | 0.68249 |
| 2488 | I.S. | 0.68249 | 2489 | FANCA | 0.68244 | 2490 | I.S. | 0.68243 |
| 2491 | NTRK3 | 0.68243 | 2492 | CIC | 0.68242 | 2493 | IDH1 | 0.68241 |
| 2494 | NF1 | 0.68241 | 2495 | RBBP6 | 0.6824 | 2496 | PDGFRB | 0.68239 |
| 2497 | SF3B1 | 0.68235 | 2498 | I.S. | 0.68233 | 2499 | I.S. | 0.68233 |
| 2500 | I.S. | 0.68233 | 2501 | DDX41 | 0.68229 | 2502 | I.S. | 0.68227 |
| 2503 | I.S. | 0.68223 | 2504 | MLH1 | 0.68222 | 2505 | ATM | 0.68222 |
| 2506 | I.S. | 0.68221 | 2507 | ARID1B | 0.68219 | 2508 | I.S. | 0.68217 |
| 2509 | I.S. | 0.68214 | 2510 | I.S. | 0.68213 | 2511 | DDR2 | 0.68213 |
| 2512 | I.S. | 0.68211 | 2513 | I.S. | 0.68208 | 2514 | CHD2 | 0.68203 |
| 2515 | CCND1 | 0.68202 | 2516 | FGF23 | 0.68202 | 2517 | I.S. | 0.68201 |
| 2518 | I.S. | 0.682 | 2519 | I.S. | 0.682 | 2520 | I.S. | 0.68197 |
| 2521 | I.S. | 0.68197 | 2522 | I.S. | 0.68196 | 2523 | DAXX | 0.6819 |
| 2524 | MLH1 | 0.6819 | 2525 | STK11 | 0.68187 | 2526 | CDAN1 | 0.68182 |
| 2527 | I.S. | 0.68178 | 2528 | I.S. | 0.68178 | 2529 | I.S. | 0.68178 |
| 2530 | I.S. | 0.68178 | 2531 | I.S. | 0.68175 | 2532 | KMT2D | 0.68169 |
| 2533 | EZH2 | 0.68169 | 2534 | I.S. | 0.68166 | 2535 | ASXL1 | 0.68166 |
| 2536 | CTC1 | 0.68158 | 2537 | I.S. | 0.68157 | 2538 | I.S. | 0.68152 |
| 2539 | I.S. | 0.6815 | 2540 | GATA6 | 0.68149 | 2541 | I.S. | 0.68145 |
| 2542 | EMSY | 0.68145 | 2543 | I.S. | 0.68142 | 2544 | I.S. | 0.68137 |
| 2545 | GFI1B | 0.68131 | 2546 | XRCC2 | 0.68128 | 2547 | I.S. | 0.68124 |
| 2548 | TERF2 | 0.6812 | 2549 | GNAQ | 0.68119 | 2550 | CIC | 0.68117 |
| 2551 | DDX11 | 0.68116 | 2552 | CREBBP | 0.68116 | 2553 | RICTOR | 0.68115 |
| 2554 | I.S. | 0.68114 | 2555 | I.S. | 0.68113 | 2556 | I.S. | 0.6811 |
| 2557 | I.S. | 0.68107 | 2558 | I.S. | 0.68107 | 2559 | I.S. | 0.68107 |
| 2560 | MLH1 | 0.68104 | 2561 | DDX11 | 0.68104 | 2562 | GLI1 | 0.68101 |
| 2563 | CBLB | 0.68099 | 2564 | I.S. | 0.68099 | 2565 | I.S. | 0.68095 |
| 2566 | LRP1B | 0.68095 | 2567 | I.S. | 0.68095 | 2568 | I.S. | 0.68095 |
| 2569 | DICER1 | 0.68095 | 2570 | I.S. | 0.68092 | 2571 | I.S. | 0.68092 |
| 2572 | I.S. | 0.68092 | 2573 | I.S. | 0.68092 | 2574 | EGFR | 0.68092 |
| 2575 | I.S. | 0.6809 | 2576 | I.S. | 0.68088 | 2577 | NBPF20, | 0.68087 |
| | | | | | | | NBPF19, | |
| | | | | | | | NBPF10, | |
| | | | | | | | RBM8A | |
| 2578 | PLCG2 | 0.68086 | 2579 | SLX4 | 0.68084 | 2580 | PMS2 | 0.68084 |
| 2581 | I.S. | 0.68074 | 2582 | I.S. | 0.68074 | 2583 | I.S. | 0.68074 |
| 2584 | PRKN | 0.68074 | 2585 | DDR2 | 0.68072 | 2586 | I.S. | 0.68071 |
| 2587 | LRP1B | 0.68071 | 2588 | PTCH1 | 0.68071 | 2589 | I.S. | 0.68071 |
| 2590 | I.S. | 0.68068 | 2591 | I.S. | 0.68068 | 2592 | I.S. | 0.68063 |
| 2593 | RET | 0.68063 | 2594 | I.S. | 0.68063 | 2595 | I.S. | 0.68062 |
| 2596 | I.S. | 0.68059 | 2597 | I.S. | 0.68057 | 2598 | I.S. | 0.68054 |
| 2599 | AXL | 0.68054 | 2600 | PIK3C2B | 0.68054 | 2601 | LZTR1 | 0.68054 |
| 2602 | LRP1B | 0.68051 | 2603 | I.S. | 0.6805 | 2604 | I.S. | 0.6805 |
| 2605 | I.S. | 0.68048 | 2606 | I.S. | 0.68041 | 2607 | I.S. | 0.68038 |
| 2608 | BRIP1 | 0.68038 | 2609 | ATG2B | 0.68038 | 2610 | I.S. | 0.68032 |
| 2611 | KMT2A | 0.6803 | 2612 | LRP1B | 0.6803 | 2613 | I.S. | 0.68029 |
| 2614 | I.S. | 0.68027 | 2615 | I.S. | 0.68026 | 2616 | I.S. | 0.68025 |
| 2617 | I.S. | 0.68024 | 2618 | ARID1B | 0.68023 | 2619 | KMT2B | 0.68021 |
| 2620 | KMT2C | 0.68019 | 2621 | I.S. | 0.68018 | 2622 | I.S. | 0.68017 |
| 2623 | EED, | 0.68016 | 2624 | FLT1 | 0.68015 | 2625 | SYK | 0.68015 |
| | MIR6755 | | | | | | | |
| 2626 | DAXX | 0.6801 | 2627 | I.S. | 0.68009 | 2628 | I.S. | 0.68009 |
| 2629 | SLIT2 | 0.68009 | 2630 | I.S. | 0.68005 | 2631 | I.S. | 0.68003 |
| 2632 | CTNNA1 | 0.68 | 2633 | KEL | 0.68 | 2634 | FGFR4 | 0.67997 |
| 2635 | CUX1 | 0.67997 | 2636 | CCN6 | 0.67997 | 2637 | EPHB1 | 0.67996 |
| 2638 | I.S. | 0.67984 | 2639 | I.S. | 0.67983 | 2640 | I.S. | 0.67983 |
| 2641 | CUX1 | 0.67979 | 2642 | CDH1 | 0.67977 | 2643 | EXO1 | 0.67976 |
| 2644 | I.S. | 0.67976 | 2645 | FANCG | 0.67968 | 2646 | MCL1 | 0.67967 |
| 2647 | IKBKE | 0.67967 | 2648 | I.S. | 0.67964 | 2649 | I.S. | 0.67964 |
| 2650 | I.S. | 0.67962 | 2651 | I.S. | 0.67962 | 2652 | I.S. | 0.67962 |
| 2653 | I.S. | 0.6796 | 2654 | BCL2 | 0.67959 | 2655 | BLM | 0.67953 |
| 2656 | I.S. | 0.67953 | 2657 | I.S. | 0.6795 | 2658 | I.S. | 0.67944 |
| 2659 | NSD1 | 0.67944 | 2660 | I.S. | 0.67944 | 2661 | I.S. | 0.67941 |
| 2662 | CHEK1 | 0.6794 | 2663 | I.S. | 0.6794 | 2664 | I.S. | 0.67939 |
| 2665 | LYST | 0.67938 | 2666 | PIK3CA | 0.67938 | 2667 | I.S. | 0.67935 |
| 2668 | SLX4 | 0.67935 | 2669 | DDR2 | 0.67934 | 2670 | I.S. | 0.67933 |
| 2671 | I.S. | 0.67927 | 2672 | I.S. | 0.67923 | 2673 | NF1 | 0.6792 |
| 2674 | LYST | 0.67918 | 2675 | SOCS1 | 0.67918 | 2676 | I.S. | 0.67914 |
| 2677 | I.S. | 0.67909 | 2678 | LYST | 0.67905 | 2679 | IGF1R | 0.67902 |
| 2680 | I.S. | 0.67899 | 2681 | POT1 | 0.67899 | 2682 | I.S. | 0.67897 |
| 2683 | I.S. | 0.67897 | 2684 | TBX3 | 0.67893 | 2685 | I.S. | 0.67893 |
| 2686 | I.S. | 0.67893 | 2687 | I.S. | 0.67891 | 2688 | I.S. | 0.6789 |
| 2689 | ERBB3 | 0.6789 | 2690 | ARID1B | 0.6789 | 2691 | RBBP6 | 0.67887 |
| 2692 | I.S. | 0.67887 | 2693 | I.S. | 0.67885 | 2694 | FAT1 | 0.67882 |
| 2695 | FANCM | 0.67882 | 2696 | ACD | 0.67882 | 2697 | I.S. | 0.67878 |
| 2698 | SPTA1 | 0.67875 | 2699 | MCL1 | 0.67873 | 2700 | RNF43 | 0.67873 |
| 2701 | MET | 0.67872 | 2702 | MAP2K4 | 0.67869 | 2703 | I.S. | 0.67866 |
| 2704 | FLT1 | 0.6786 | 2705 | I.S. | 0.67858 | 2706 | I.S. | 0.67852 |
| 2707 | I.S. | 0.67852 | 2708 | CSF3R | 0.67852 | 2709 | TSC1 | 0.67852 |
| 2710 | I.S. | 0.6785 | 2711 | I.S. | 0.67849 | 2712 | I.S. | 0.67843 |
| 2713 | I.S. | 0.67842 | 2714 | RHEB | 0.67842 | 2715 | KMT2D | 0.6784 |
| 2716 | TRAF3 | 0.6784 | 2717 | SPEN | 0.67839 | 2718 | I.S. | 0.67838 |
| 2719 | I.S. | 0.67836 | 2720 | I.S. | 0.67836 | 2721 | I.S. | 0.67836 |
| 2722 | I.S. | 0.67836 | 2723 | I.S. | 0.67828 | 2724 | FAT1 | 0.67828 |
| 2725 | I.S. | 0.67828 | 2726 | FGFR2 | 0.67828 | 2727 | TBX3 | 0.67828 |
| 2728 | CREBBP | 0.67828 | 2729 | CDH1 | 0.67825 | 2730 | GRIN2A | 0.67824 |
| 2731 | I.S. | 0.67823 | 2732 | ABL2 | 0.67822 | 2733 | IGF1R | 0.67819 |
| 2734 | I.S. | 0.67817 | 2735 | EPHB1 | 0.67816 | 2736 | CHEK2 | 0.67816 |
| 2737 | I.S. | 0.67814 | 2738 | I.S. | 0.67812 | 2739 | I.S. | 0.67812 |
| 2740 | TET2 | 0.67807 | 2741 | NTRK1 | 0.67806 | 2742 | I.S. | 0.67806 |
| 2743 | IRF2 | 0.67795 | 2744 | I.S. | 0.67793 | 2745 | ARID1B | 0.67792 |
| 2746 | I.S. | 0.67789 | 2747 | I.S. | 0.67788 | 2748 | I.S. | 0.67787 |
| 2749 | PLCG1 | 0.67786 | 2750 | PMS2 | 0.67786 | 2751 | I.S. | 0.67785 |
| 2752 | I.S. | 0.67781 | 2753 | I.S. | 0.67781 | 2754 | I.S. | 0.67771 |
| 2755 | I.S. | 0.67768 | 2756 | I.S. | 0.67767 | 2757 | I.S. | 0.67765 |
| 2758 | I.S. | 0.67765 | 2759 | STAT6 | 0.67762 | 2760 | PDK1 | 0.67762 |
| 2761 | MYD88 | 0.67762 | 2762 | RICTOR | 0.67762 | 2763 | SF3B1 | 0.67759 |
| 2764 | I.S. | 0.67757 | 2765 | I.S. | 0.67757 | 2766 | TSC2 | 0.67753 |
| 2767 | I.S. | 0.67748 | 2768 | I.S. | 0.67748 | 2769 | I.S. | 0.67745 |
| 2770 | I.S. | 0.67743 | 2771 | I.S. | 0.67742 | 2772 | ARID2 | 0.67742 |
| 2773 | PIK3C2B | 0.67741 | 2774 | NTRK2 | 0.67738 | 2775 | I.S. | 0.67737 |
| 2776 | SOX9 | 0.67733 | 2777 | I.S. | 0.6773 | 2778 | I.S. | 0.67726 |
| 2779 | ASXL1 | 0.67724 | 2780 | I.S. | 0.67724 | 2781 | BRCA2 | 0.67717 |
| 2782 | DDX11 | 0.67716 | 2783 | I.S. | 0.67713 | 2784 | TOP1 | 0.67712 |
| 2785 | I.S. | 0.6771 | 2786 | CBL | 0.6771 | 2787 | ALK | 0.67709 |
| 2788 | GATA4 | 0.67709 | 2789 | KIF23 | 0.67709 | 2790 | I.S. | 0.67707 |
| 2791 | TP53 | 0.67703 | 2792 | I.S. | 0.67701 | 2793 | I.S. | 0.67698 |
| 2794 | I.S. | 0.67694 | 2795 | PIK3C2B | 0.67688 | 2796 | STAT4 | 0.67688 |
| 2797 | I.S. | 0.67688 | 2798 | WT1 | 0.67688 | 2799 | JAK2 | 0.67688 |
| 2800 | I.S. | 0.67685 | 2801 | ERBB3 | 0.67685 | 2802 | I.S. | 0.67683 |
| 2803 | I.S. | 0.67681 | 2804 | I.S. | 0.6768 | 2805 | RPTOR | 0.67679 |
| 2806 | I.S. | 0.67677 | 2807 | I.S. | 0.67677 | 2808 | FAT1 | 0.67673 |
| 2809 | I.S. | 0.67672 | 2810 | I.S. | 0.67671 | 2811 | I.S. | 0.67669 |
| 2812 | I.S. | 0.67668 | 2813 | I.S. | 0.67667 | 2814 | I.S. | 0.67664 |
| 2815 | I.S. | 0.67664 | 2816 | NSD1 | 0.67664 | 2817 | NSD2 | 0.67658 |
| 2818 | I.S. | 0.67658 | 2819 | I.S. | 0.67653 | 2820 | PREX2 | 0.67652 |
| 2821 | I.S. | 0.6765 | 2822 | I.S. | 0.67647 | 2823 | I.S. | 0.67647 |
| 2824 | I.S. | 0.67645 | 2825 | I.S. | 0.67645 | 2826 | I.S. | 0.67645 |
| 2827 | I.S. | 0.67645 | 2828 | I.S. | 0.67645 | 2829 | I.S. | 0.67643 |
| 2830 | IGF1R | 0.67643 | 2831 | I.S. | 0.67641 | 2832 | I.S. | 0.67636 |
| 2833 | I.S. | 0.67634 | 2834 | I.S. | 0.67632 | 2835 | I.S. | 0.67628 |
| 2836 | I.S. | 0.67627 | 2837 | BRD4 | 0.67626 | 2838 | I.S. | 0.67624 |
| 2839 | I.S. | 0.67624 | 2840 | I.S. | 0.67623 | 2841 | CDC73 | 0.67623 |
| 2842 | I.S. | 0.67622 | 2843 | I.S. | 0.67622 | 2844 | I.S. | 0.67614 |
| 2845 | I.S. | 0.67613 | 2846 | I.S. | 0.67612 | 2847 | I.S. | 0.67611 |
| 2848 | I.S. | 0.67604 | 2849 | GATA6 | 0.67602 | 2850 | NSD2 | 0.67602 |
| 2851 | I.S. | 0.67602 | 2852 | BCR | 0.67598 | 2853 | I.S. | 0.67598 |
| 2854 | I.S. | 0.67598 | 2855 | I.S. | 0.67597 | 2856 | SPTA1 | 0.67596 |
| 2857 | I.S. | 0.67592 | 2858 | TERF2 | 0.6759 | 2859 | ERBB3 | 0.6759 |
| 2860 | NBN | 0.67589 | 2861 | GLI1 | 0.67587 | 2862 | MSH2 | 0.67585 |
| 2863 | I.S. | 0.67581 | 2864 | I.S. | 0.67579 | 2865 | ARID1A | 0.67578 |
| 2866 | PIK3C2B | 0.67578 | 2867 | I.S. | 0.67578 | 2868 | I.S. | 0.67578 |
| 2869 | CARD11 | 0.67576 | 2870 | I.S. | 0.67576 | 2871 | I.S. | 0.67576 |
| 2872 | PSTPIP1 | 0.67576 | 2873 | I.S. | 0.67574 | 2874 | I.S. | 0.67571 |
| 2875 | I.S. | 0.67567 | 2876 | I.S. | 0.67565 | 2877 | LPIN2 | 0.67565 |
| 2878 | CIC | 0.67561 | 2879 | I.S. | 0.67551 | 2880 | RANBP2 | 0.67549 |
| 2881 | ERCC4 | 0.67548 | 2882 | TERF2 | 0.67546 | 2883 | I.S. | 0.67545 |
| 2884 | PTEN | 0.67545 | 2885 | I.S. | 0.67545 | 2886 | LRP1B | 0.67545 |
| 2887 | RICTOR | 0.67545 | 2888 | I.S. | 0.67541 | 2889 | SOX2-OT, | 0.6754 |
| | | | | | | | SOX2 | |
| 2890 | I.S. | 0.6754 | 2891 | SPTA1 | 0.6754 | 2892 | I.S. | 0.67537 |
| 2893 | I.S. | 0.67537 | 2894 | KMT2A | 0.67536 | 2895 | — | 0.67536 |
| 2896 | I.S. | 0.67535 | 2897 | ROS1 | 0.6753 | 2898 | I.S. | 0.67527 |
| 2899 | I.S. | 0.67519 | 2900 | SPTA1 | 0.67519 | 2901 | I.S. | 0.67519 |
| 2902 | MVK | 0.67513 | 2903 | BLM | 0.67513 | 2904 | BRCA2 | 0.67512 |
| 2905 | LPIN2 | 0.67507 | 2906 | RAD51D | 0.67498 | 2907 | RTEL1 | 0.67492 |
| 2908 | EMSY | 0.67492 | 2909 | I.S. | 0.67486 | 2910 | I.S. | 0.67486 |
| 2911 | I.S. | 0.67486 | 2912 | BCL2L2 | 0.67486 | 2913 | I.S. | 0.67483 |
| 2914 | I.S. | 0.67479 | 2915 | AXL | 0.67478 | 2916 | I.S. | 0.67474 |
| 2917 | I.S. | 0.67474 | 2918 | PPM1D | 0.67474 | 2919 | I.S. | 0.67472 |
| 2920 | I.S. | 0.67472 | 2921 | POLE | 0.67466 | 2922 | I.S. | 0.67464 |
| 2923 | I.S. | 0.67464 | 2924 | I.S. | 0.67463 | 2925 | ACVR1B | 0.67462 |
| 2926 | I.S. | 0.67462 | 2927 | SBF2 | 0.67453 | 2928 | CIC | 0.67453 |
| 2929 | I.S. | 0.67443 | 2930 | CUX1 | 0.67442 | 2931 | I.S. | 0.67439 |
| 2932 | I.S. | 0.67439 | 2933 | STK11 | 0.67439 | 2934 | FGFR4 | 0.67436 |
| 2935 | I.S. | 0.67433 | 2936 | I.S. | 0.67433 | 2937 | I.S. | 0.67433 |
| 2938 | I.S. | 0.67431 | 2939 | I.S. | 0.6743 | 2940 | NME1 | 0.6743 |
| 2941 | BARD1 | 0.67429 | 2942 | TERC | 0.67424 | 2943 | DICER1 | 0.67424 |
| 2944 | ERBB2 | 0.67421 | 2945 | I.S. | 0.67421 | 2946 | SMARCB1 | 0.67417 |
| 2947 | KMT2A | 0.67415 | 2948 | I.S. | 0.67409 | 2949 | RICTOR | 0.67408 |
| 2950 | TP53 | 0.67408 | 2951 | I.S. | 0.67406 | 2952 | I.S. | 0.67403 |
| 2953 | SETD2 | 0.67397 | 2954 | I.S. | 0.67397 | 2955 | I.S. | 0.67396 |
| 2956 | BARD1 | 0.67395 | 2957 | GLI2 | 0.67392 | 2958 | ERCC4 | 0.67391 |
| 2959 | I.S. | 0.6739 | 2960 | PIK3R2 | 0.67389 | 2961 | GLI1 | 0.67388 |
| 2962 | I.S. | 0.67388 | 2963 | I.S. | 0.67384 | 2964 | I.S. | 0.67379 |
| 2965 | I.S. | 0.67379 | 2966 | SRC | 0.67376 | 2967 | PIK3CB | 0.67376 |
| 2968 | AXIN2 | 0.67376 | 2969 | I.S. | 0.67376 | 2970 | I.S. | 0.67373 |
| 2971 | FLT1 | 0.6737 | 2972 | I.S. | 0.67368 | 2973 | MYCNOS, | 0.67368 |
| | | | | | | | MYCN, MYCN | |
| 2974 | I.S. | 0.67365 | 2975 | MEF2B | 0.67362 | 2976 | IKZF3 | 0.67361 |
| 2977 | I.S. | 0.6736 | 2978 | I.S. | 0.67358 | 2979 | I.S. | 0.67355 |
| 2980 | I.S. | 0.67354 | 2981 | PIK3C2B | 0.67349 | 2982 | TSC2 | 0.67347 |
| 2983 | I.S. | 0.67346 | 2984 | I.S. | 0.67343 | 2985 | CDC73 | 0.67341 |
| 2986 | EPHA7 | 0.67338 | 2987 | SBF2 | 0.67337 | 2988 | KMT2D | 0.67334 |
| 2989 | MTOR | 0.67331 | 2990 | I.S. | 0.67328 | 2991 | I.S. | 0.67327 |
| 2992 | I.S. | 0.67325 | 2993 | I.S. | 0.67324 | 2994 | I.S. | 0.67323 |
| 2995 | FLT4 | 0.67323 | 2996 | EPHB1 | 0.67323 | 2997 | I.S. | 0.67313 |
| 2998 | CARD11 | 0.67311 | 2999 | I.S. | 0.67311 | 3000 | I.S. | 0.67308 |
| 3001 | BRCA2 | 0.67307 | 3002 | I.S. | 0.67306 | 3003 | FGFR3 | 0.67305 |
| 3004 | EGLN1 | 0.67305 | 3005 | I.S. | 0.67305 | 3006 | JAK3 | 0.67305 |
| 3007 | I.S. | 0.67303 | 3008 | I.S. | 0.67303 | 3009 | I.S. | 0.67299 |
| 3010 | CTC1 | 0.67299 | 3011 | I.S. | 0.67297 | 3012 | HOXA11 | 0.67296 |
| 3013 | I.S. | 0.67293 | 3014 | NSD1 | 0.67293 | 3015 | I.S. | 0.67293 |
| 3016 | I.S. | 0.67281 | 3017 | I.S. | 0.67281 | 3018 | I.S. | 0.67281 |
| 3019 | I.S. | 0.67281 | 3020 | I.S. | 0.67281 | 3021 | I.S. | 0.67281 |
| 3022 | CDK8 | 0.67281 | 3023 | ATM | 0.67277 | 3024 | LYST | 0.67275 |
| 3025 | I.S. | 0.67272 | 3026 | NSD1 | 0.67272 | 3027 | I.S. | 0.67269 |
| 3028 | I.S. | 0.67261 | 3029 | I.S. | 0.6726 | 3030 | I.S. | 0.67257 |
| 3031 | I.S. | 0.67257 | 3032 | I.S. | 0.67255 | 3033 | I.S. | 0.6725 |
| 3034 | I.S. | 0.6725 | 3035 | I.S. | 0.67247 | 3036 | CDKN2A | 0.67243 |
| 3037 | I.S. | 0.67243 | 3038 | LYST | 0.6724 | 3039 | I.S. | 0.6724 |
| 3040 | EXO1 | 0.67237 | 3041 | I.S. | 0.67236 | 3042 | PLCG2 | 0.6723 |
| 3043 | ESR1 | 0.67228 | 3044 | I.S. | 0.67228 | 3045 | I.S. | 0.67228 |
| 3046 | I.S. | 0.67226 | 3047 | ERBB3 | 0.67225 | 3048 | I.S. | 0.67225 |
| 3049 | TSC2 | 0.67225 | 3050 | I.S. | 0.67224 | 3051 | I.S. | 0.67219 |
| 3052 | QKI | 0.67219 | 3053 | TAL1 | 0.67219 | 3054 | PMS1 | 0.67219 |
| 3055 | BRIP1 | 0.67215 | 3056 | ADA | 0.67215 | 3057 | BRCA2 | 0.67209 |
| 3058 | I.S. | 0.67207 | 3059 | I.S. | 0.67207 | 3060 | I.S. | 0.67205 |
| 3061 | ERBB2 | 0.67204 | 3062 | TSC2 | 0.67204 | 3063 | MAP3K14 | 0.67204 |
| 3064 | FANCA | 0.67199 | 3065 | JAK1 | 0.67198 | 3066 | I.S. | 0.67198 |
| 3067 | I.S. | 0.67195 | 3068 | I.S. | 0.67193 | 3069 | I.S. | 0.67193 |
| 3070 | I.S. | 0.67189 | 3071 | I.S. | 0.67188 | 3072 | DDX41 | 0.67186 |
| 3073 | I.S. | 0.6718 | 3074 | I.S. | 0.67179 | 3075 | FBXW7 | 0.67177 |
| 3076 | CIC | 0.67175 | 3077 | ARID1B | 0.67174 | 3078 | I.S. | 0.67173 |
| 3079 | I.S. | 0.67173 | 3080 | NLRP3 | 0.67171 | 3081 | I.S. | 0.67171 |
| 3082 | AKT2 | 0.67168 | 3083 | I.S. | 0.67166 | 3084 | I.S. | 0.67165 |
| 3085 | I.S. | 0.67165 | 3086 | RIT1 | 0.67163 | 3087 | EXO1 | 0.67163 |
| 3088 | I.S. | 0.6716 | 3089 | FLT3 | 0.6716 | 3090 | RUNX1T1 | 0.67159 |
| 3091 | CBFB | 0.67157 | 3092 | GRIN2A | 0.6715 | 3093 | I.S. | 0.67148 |
| 3094 | GLI2 | 0.67148 | 3095 | I.S. | 0.67141 | 3096 | I.S. | 0.67139 |
| 3097 | FLT3 | 0.67136 | 3098 | I.S. | 0.67127 | 3099 | ABL2 | 0.67127 |
| 3100 | I.S. | 0.67124 | 3101 | I.S. | 0.67122 | 3102 | I.S. | 0.67122 |
| 3103 | I.S. | 0.67122 | 3104 | I.S. | 0.67122 | 3105 | CDAN1 | 0.67121 |
| 3106 | — | 0.67121 | 3107 | RAD50, | 0.67117 | 3108 | HAX1 | 0.67116 |
| | | | | TH2LCRR | | | | |
| 3109 | FANCF | 0.67116 | 3110 | I.S. | 0.67114 | 3111 | I.S. | 0.67112 |
| 3112 | I.S. | 0.6711 | 3113 | DICER1 | 0.67108 | 3114 | I.S. | 0.67106 |
| 3115 | SPTA1 | 0.67106 | 3116 | ATM | 0.67106 | 3117 | NOTCH1 | 0.67101 |
| 3118 | I.S. | 0.671 | 3119 | I.S. | 0.671 | 3120 | I.S. | 0.67097 |
| 3121 | LYST | 0.67097 | 3122 | I.S. | 0.67096 | 3123 | KDM5A | 0.67093 |
| 3124 | IRS2 | 0.67091 | 3125 | FGFR1 | 0.67088 | 3126 | POLE | 0.67088 |
| 3127 | I.S. | 0.67086 | 3128 | I.S. | 0.67086 | 3129 | I.S. | 0.67086 |
| 3130 | I.S. | 0.67077 | 3131 | I.S. | 0.67077 | 3132 | NUP93 | 0.67076 |
| 3133 | KMT2C | 0.67075 | 3134 | I.S. | 0.67075 | 3135 | NSD2 | 0.67074 |
| 3136 | KMT2D | 0.67067 | 3137 | I.S. | 0.67067 | 3138 | I.S. | 0.67067 |
| 3139 | I.S. | 0.67067 | 3140 | NTRK1 | 0.67064 | 3141 | NF1 | 0.67062 |
| 3142 | I.S. | 0.67061 | 3143 | I.S. | 0.67061 | 3144 | KDR | 0.67059 |
| 3145 | ALK | 0.67055 | 3146 | FAT1 | 0.67055 | 3147 | I.S. | 0.67055 |
| 3148 | I.S. | 0.67054 | 3149 | NLRP3 | 0.67052 | 3150 | STAT4 | 0.67052 |
| 3151 | BRCA2 | 0.67052 | 3152 | I.S. | 0.67049 | 3153 | I.S. | 0.67048 |
| 3154 | DOT1L | 0.67047 | 3155 | I.S. | 0.67047 | 3156 | I.S. | 0.67042 |
| 3157 | ARID1B | 0.67041 | 3158 | TOP1 | 0.67041 | 3159 | I.S. | 0.67041 |
| 3160 | I.S. | 0.67039 | 3161 | I.S. | 0.67038 | 3162 | I.S. | 0.67036 |
| 3163 | LYST | 0.67035 | 3164 | I.S. | 0.67034 | 3165 | I.S. | 0.67034 |
| 3166 | I.S. | 0.67034 | 3167 | FBXW7 | 0.67031 | 3168 | I.S. | 0.6703 |
| 3169 | EP300 | 0.67023 | 3170 | TP53 | 0.67023 | 3171 | ELANE | 0.67023 |
| 3172 | I.S. | 0.67018 | 3173 | I.S. | 0.67014 | 3174 | I.S. | 0.67014 |
| 3175 | I.S. | 0.67012 | 3176 | I.S. | 0.67012 | 3177 | BLM | 0.6701 |
| 3178 | KMT2D | 0.67001 | 3179 | I.S. | 0.67001 | 3180 | PTCH1 | 0.66998 |
| 3181 | I.S. | 0.66996 | 3182 | I.S. | 0.66993 | 3183 | I.S. | 0.66991 |
| 3184 | I.S. | 0.66987 | 3185 | I.S. | 0.66987 | 3186 | I.S. | 0.66987 |
| 3187 | I.S. | 0.66986 | 3188 | I.S. | 0.66985 | 3189 | I.S. | 0.66985 |
| 3190 | I.S. | 0.66981 | 3191 | TSC2 | 0.66981 | 3192 | I.S. | 0.66981 |
| 3193 | I.S. | 0.66978 | 3194 | I.S. | 0.66977 | 3195 | EPHA7 | 0.66972 |
| 3196 | CDK8 | 0.66972 | 3197 | ANKRD26 | 0.66969 | 3198 | ARID2 | 0.66969 |
| 3199 | JAK2 | 0.66966 | 3200 | ARID2 | 0.66965 | 3201 | I.S. | 0.66963 |
| 3202 | I.S. | 0.66963 | 3203 | I.S. | 0.66963 | 3204 | I.S. | 0.66963 |
| 3205 | KDM5A | 0.66957 | 3206 | I.S. | 0.66955 | 3207 | I.S. | 0.66955 |
| 3208 | I.S. | 0.66955 | 3209 | I.S. | 0.66955 | 3210 | I.S. | 0.66952 |
| 3211 | — | 0.66951 | 3212 | I.S. | 0.66951 | 3213 | I.S. | 0.66951 |
| 3214 | KDM5A | 0.66951 | 3215 | KMT2B | 0.66951 | 3216 | CARD11 | 0.6695 |
| 3217 | LYST | 0.66949 | 3218 | KMT2D | 0.66948 | 3219 | KMT2C | 0.66942 |
| 3220 | I.S. | 0.66941 | 3221 | I.S. | 0.66939 | 3222 | I.S. | 0.66937 |
| 3223 | I.S. | 0.66936 | 3224 | I.S. | 0.66935 | 3225 | I.S. | 0.66934 |
| 3226 | I.S. | 0.66933 | 3227 | I.S. | 0.66927 | 3228 | I.S. | 0.66927 |
| 3229 | CARD11 | 0.66924 | 3230 | I.S. | 0.66923 | 3231 | I.S. | 0.66922 |
| 3232 | SPTA1 | 0.66922 | 3233 | I.S. | 0.66919 | 3234 | I.S. | 0.66915 |
| 3235 | SUFU | 0.66913 | 3236 | I.S. | 0.66908 | 3237 | I.S. | 0.66908 |
| 3238 | AURKC | 0.66907 | 3239 | I.S. | 0.66906 | 3240 | APC | 0.66903 |
| 3241 | I.S. | 0.66903 | 3242 | CSF3R | 0.66902 | 3243 | I.S. | 0.669 |
| 3244 | I.S. | 0.66898 | 3245 | LYST | 0.66895 | 3246 | ERBB3 | 0.66895 |
| 3247 | I.S. | 0.66894 | 3248 | NTRK1 | 0.66889 | 3249 | TSC2 | 0.66887 |
| 3250 | I.S. | 0.66887 | 3251 | I.S. | 0.66887 | 3252 | I.S. | 0.66886 |
| 3253 | I.S. | 0.66886 | 3254 | SMC3 | 0.66883 | 3255 | NTRK1 | 0.66881 |
| 3256 | ERBB3 | 0.6688 | 3257 | GNAQ | 0.66878 | 3258 | I.S. | 0.66875 |
| 3259 | LYST | 0.66875 | 3260 | LRP1B | 0.66874 | 3261 | I.S. | 0.66871 |
| 3262 | I.S. | 0.66869 | 3263 | FGFR4 | 0.66869 | 3264 | CDKN2B | 0.66869 |
| 3265 | PRKAR1A | 0.66869 | 3266 | BRCA2 | 0.66862 | 3267 | I.S. | 0.66857 |
| 3268 | I.S. | 0.66854 | 3269 | I.S. | 0.66853 | 3270 | I.S. | 0.66853 |
| 3271 | ZNF276, | 0.66849 | 3272 | I.S. | 0.66844 | 3273 | TSC2 | 0.66842 |
| | FANCA | | | | | | | |
| 3274 | I.S. | 0.66837 | 3275 | LPIN2 | 0.66836 | 3276 | FGFR2 | 0.66833 |
| 3277 | FLT3 | 0.66832 | 3278 | I.S. | 0.66831 | 3279 | I.S. | 0.66829 |
| 3280 | I.S. | 0.66829 | 3281 | I.S. | 0.66829 | 3282 | I.S. | 0.66829 |
| 3283 | I.S. | 0.66823 | 3284 | ERBB4 | 0.6682 | 3285 | I.S. | 0.6682 |
| 3286 | I.S. | 0.66819 | 3287 | I.S. | 0.66818 | 3288 | LRP1B | 0.66815 |
| 3289 | I.S. | 0.66813 | 3290 | GNAS | 0.66812 | 3291 | I.S. | 0.66812 |
| 3292 | I.S. | 0.66809 | 3293 | I.S. | 0.66809 | 3294 | I.S. | 0.66808 |
| 3295 | I.S. | 0.66804 | 3296 | I.S. | 0.66804 | 3297 | I.S. | 0.66804 |
| 3298 | I.S. | 0.66804 | 3299 | ERG | 0.66803 | 3300 | GATA2 | 0.66796 |
| 3301 | I.S. | 0.66796 | 3302 | I.S. | 0.66796 | 3303 | I.S. | 0.66792 |
| 3304 | I.S. | 0.66791 | 3305 | I.S. | 0.6679 | 3306 | I.S. | 0.66788 |
| 3307 | FGFR4 | 0.6678 | 3308 | FGFR4 | 0.66777 | 3309 | I.S. | 0.66776 |
| 3310 | FLT1 | 0.66776 | 3311 | JAK1 | 0.66774 | 3312 | I.S. | 0.66774 |
| 3313 | I.S. | 0.66772 | 3314 | NLRP3 | 0.66771 | 3315 | I.S. | 0.6677 |
| 3316 | I.S. | 0.66768 | 3317 | NF1 | 0.66763 | 3318 | I.S. | 0.66759 |
| 3319 | I.S. | 0.66758 | 3320 | I.S. | 0.66756 | 3321 | I.S. | 0.66756 |
| 3322 | I.S. | 0.66749 | 3323 | I.S. | 0.66749 | 3324 | I.S. | 0.66747 |
| 3325 | LRP1B | 0.66746 | 3326 | I.S. | 0.66746 | 3327 | SMARCA4 | 0.66744 |
| 3328 | BRAF | 0.66738 | 3329 | PREX2 | 0.66729 | 3330 | I.S. | 0.66727 |
| 3331 | I.S. | 0.66727 | 3332 | I.S. | 0.66727 | 3333 | I.S. | 0.66725 |
| 3334 | I.S. | 0.66725 | 3335 | I.S. | 0.66725 | 3336 | I.S. | 0.66725 |
| 3337 | I.S. | 0.66725 | 3338 | I.S. | 0.66725 | 3339 | I.S. | 0.66725 |
| 3340 | NSD1 | 0.66725 | 3341 | I.S. | 0.66725 | 3342 | I.S. | 0.66719 |
| 3343 | PPM1D | 0.66717 | 3344 | I.S. | 0.66717 | 3345 | TSC2 | 0.66717 |
| 3346 | I.S. | 0.66717 | 3347 | ACVR1B | 0.66717 | 3348 | I.S. | 0.66715 |
| 3349 | I.S. | 0.66713 | 3350 | I.S. | 0.66713 | 3351 | I.S. | 0.66713 |
| 3352 | I.S. | 0.66713 | 3353 | FLT1 | 0.66711 | 3354 | TERF1 | 0.66705 |
| 3355 | I.S. | 0.66705 | 3356 | MSH6 | 0.66701 | 3357 | I.S. | 0.66701 |
| 3358 | EED | 0.66699 | 3359 | I.S. | 0.66698 | 3360 | I.S. | 0.66697 |
| 3361 | CD79B | 0.66697 | 3362 | SEC23B | 0.66697 | 3363 | U2AF1, | 0.66697 |
| | | | | | | | U2AF1L5 | |
| 3364 | I.S. | 0.66694 | 3365 | I.S. | 0.66694 | 3366 | I.S. | 0.66694 |
| 3367 | I.S. | 0.66693 | 3368 | I.S. | 0.66692 | 3369 | I.S. | 0.66692 |
| 3370 | I.S. | 0.66691 | 3371 | I.S. | 0.66691 | 3372 | I.S. | 0.66687 |
| 3373 | I.S. | 0.66685 | 3374 | I.S. | 0.66684 | 3375 | SETBP1 | 0.66681 |
| 3376 | I.S. | 0.66676 | 3377 | TOP1 | 0.66676 | 3378 | GNAS | 0.66672 |
| 3379 | I.S. | 0.66672 | 3380 | AXL | 0.66672 | 3381 | I.S. | 0.6667 |
| 3382 | I.S. | 0.66669 | 3383 | I.S. | 0.66669 | 3384 | FGF10 | 0.66669 |
| 3385 | TSC1 | 0.66664 | 3386 | AXL | 0.66664 | 3387 | I.S. | 0.66661 |
| 3388 | I.S. | 0.66658 | 3389 | CDK4 | 0.66651 | 3390 | I.S. | 0.66651 |
| 3391 | I.S. | 0.66651 | 3392 | KDM5A | 0.6665 | 3393 | MAP3K14 | 0.66649 |
| 3394 | IRF2 | 0.66648 | 3395 | I.S. | 0.66637 | 3396 | RAD21 | 0.66636 |
| 3397 | I.S. | 0.66636 | 3398 | I.S. | 0.66635 | 3399 | I.S. | 0.66631 |
| 3400 | ARID1B | 0.66631 | 3401 | I.S. | 0.66631 | 3402 | RBBP6 | 0.66628 |
| 3403 | LIG4 | 0.66627 | 3404 | CTNNA1 | 0.66625 | 3405 | I.S. | 0.66623 |
| 3406 | I.S. | 0.66623 | 3407 | I.S. | 0.66623 | 3408 | CIC | 0.66619 |
| 3409 | DDR2 | 0.66619 | 3410 | I.S. | 0.66615 | 3411 | I.S. | 0.66614 |
| 3412 | I.S. | 0.66613 | 3413 | I.S. | 0.6661 | 3414 | NBPF20, | 0.6661 |
| | | | | | | | NBPF19, | |
| | | | | | | | NBPF10, | |
| | | | | | | | RBM8A | |
| 3415 | I.S. | 0.6661 | 3416 | I.S. | 0.6661 | 3417 | CREBBP | 0.66608 |
| 3418 | I.S. | 0.66603 | 3419 | I.S. | 0.66602 | 3420 | I.S. | 0.66601 |
| 3421 | I.S. | 0.66599 | 3422 | I.S. | 0.66599 | 3423 | NSD1 | 0.66598 |
| 3424 | I.S. | 0.66594 | 3425 | I.S. | 0.66593 | 3426 | KMT2B | 0.66593 |
| 3427 | I.S. | 0.66592 | 3428 | I.S. | 0.66592 | 3429 | I.S. | 0.66591 |
| 3430 | I.S. | 0.66591 | 3431 | I.S. | 0.66591 | 3432 | I.S. | 0.6659 |
| 3433 | I.S. | 0.66587 | 3434 | CALR | 0.66583 | 3435 | KDM5C | 0.66578 |
| 3436 | I.S. | 0.66574 | 3437 | RAD50, | 0.66574 | 3438 | I.S. | 0.66574 |
| | | | | TH2LCRR | | | | |
| 3439 | I.S. | 0.66572 | 3440 | BCORL1 | 0.66571 | 3441 | I.S. | 0.6657 |
| 3442 | KAT6A | 0.66568 | 3443 | I.S. | 0.66566 | 3444 | I.S. | 0.66565 |
| 3445 | I.S. | 0.66563 | 3446 | FANCA | 0.66562 | 3447 | EPHA7 | 0.66556 |
| 3448 | HRAS | 0.66553 | 3449 | I.S. | 0.66552 | 3450 | I.S. | 0.66544 |
| 3451 | GEN1 | 0.66539 | 3452 | SLX4 | 0.66539 | 3453 | LRP1B | 0.66538 |
| 3454 | BRCA1 | 0.66536 | 3455 | I.S. | 0.66534 | 3456 | I.S. | 0.66534 |
| 3457 | I.S. | 0.66534 | 3458 | I.S. | 0.66534 | 3459 | I.S. | 0.66533 |
| 3460 | I.S. | 0.66532 | 3461 | CDC73 | 0.6653 | 3462 | KMT2B | 0.6653 |
| 3463 | ACD | 0.66528 | 3464 | EGLN1 | 0.66527 | 3465 | I.S. | 0.66525 |
| 3466 | CDKN1A | 0.66524 | 3467 | PTCH1 | 0.66524 | 3468 | I.S. | 0.66524 |
| 3469 | I.S. | 0.66523 | 3470 | I.S. | 0.66523 | 3471 | I.S. | 0.66523 |
| 3472 | GATA3 | 0.66521 | 3473 | I.S. | 0.6652 | 3474 | CARD11 | 0.66515 |
| 3475 | MAGI2 | 0.66515 | 3476 | I.S. | 0.66514 | 3477 | I.S. | 0.66511 |
| 3478 | I.S. | 0.66511 | 3479 | I.S. | 0.66511 | 3480 | I.S. | 0.66508 |
| 3481 | I.S. | 0.66506 | 3482 | I.S. | 0.66497 | 3483 | GSK3B | 0.66495 |
| 3484 | I.S. | 0.66492 | 3485 | I.S. | 0.66487 | 3486 | I.S. | 0.66487 |
| 3487 | I.S. | 0.66487 | 3488 | I.S. | 0.66483 | 3489 | I.S. | 0.66479 |
| 3490 | I.S. | 0.66476 | 3491 | PLCG1 | 0.66465 | 3492 | I.S. | 0.66462 |
| 3493 | I.S. | 0.66462 | 3494 | I.S. | 0.66461 | 3495 | I.S. | 0.66459 |
| 3496 | BLM | 0.66458 | 3497 | I.S. | 0.66458 | 3498 | I.S. | 0.66451 |
| 3499 | CDK12 | 0.6645 | 3500 | SRC | 0.6645 | 3501 | ERG | 0.66449 |
| 3502 | I.S. | 0.66448 | 3503 | EED | 0.66447 | 3504 | I.S. | 0.66446 |
| 3505 | I.S. | 0.66444 | 3506 | I.S. | 0.6644 | 3507 | LYN | 0.6644 |
| 3508 | EGFR | 0.66437 | 3509 | NSD1 | 0.66434 | 3510 | I.S. | 0.66429 |
| 3511 | POLE | 0.66429 | 3512 | I.S. | 0.66426 | 3513 | I.S. | 0.66426 |
| 3514 | I.S. | 0.66426 | 3515 | I.S. | 0.66424 | 3516 | I.S. | 0.66421 |
| 3517 | I.S. | 0.66417 | 3518 | I.S. | 0.66415 | 3519 | I.S. | 0.66414 |
| 3520 | TOP1 | 0.66412 | 3521 | ZNF217 | 0.66408 | 3522 | SF3B1 | 0.66407 |
| 3523 | I.S. | 0.66407 | 3524 | I.S. | 0.66407 | 3525 | I.S. | 0.66407 |
| 3526 | I.S. | 0.66405 | 3527 | STAT6 | 0.66405 | 3528 | NBN | 0.66405 |
| 3529 | I.S. | 0.66404 | 3530 | ERBB4 | 0.66402 | 3531 | CIC | 0.664 |
| 3532 | I.S. | 0.66399 | 3533 | I.S. | 0.66397 | 3534 | GABRA6 | 0.66396 |
| 3535 | RAD21 | 0.66396 | 3536 | ETV6 | 0.66396 | 3537 | ROS1 | 0.66393 |
| 3538 | MYD88 | 0.66392 | 3539 | I.S. | 0.66389 | 3540 | FANCM | 0.66385 |
| 3541 | I.S. | 0.66383 | 3542 | DDX41 | 0.66383 | 3543 | KMT2C | 0.66378 |
| 3544 | RPTOR | 0.66377 | 3545 | I.S. | 0.66375 | 3546 | I.S. | 0.66375 |
| 3547 | I.S. | 0.66375 | 3548 | I.S. | 0.66374 | 3549 | BRIP1 | 0.66373 |
| 3550 | I.S. | 0.66373 | 3551 | I.S. | 0.66372 | 3552 | I.S. | 0.66371 |
| 3553 | I.S. | 0.66371 | 3554 | DICER1 | 0.66367 | 3555 | I.S. | 0.66364 |
| 3556 | I.S. | 0.66364 | 3557 | I.S. | 0.66364 | 3558 | I.S. | 0.66363 |
| 3559 | CDH1 | 0.66363 | 3560 | I.S. | 0.6636 | 3561 | I.S. | 0.6636 |
| 3562 | NF1 | 0.66359 | 3563 | FAT1 | 0.66357 | 3564 | MYC | 0.66357 |
| 3565 | ROS1 | 0.66355 | 3566 | I.S. | 0.66353 | 3567 | I.S. | 0.66353 |
| 3568 | I.S. | 0.66353 | 3569 | I.S. | 0.66353 | 3570 | I.S. | 0.66353 |
| 3571 | I.S. | 0.66353 | 3572 | TRAF3 | 0.66342 | 3573 | FLT3 | 0.66342 |
| 3574 | I.S. | 0.66339 | 3575 | STAT4 | 0.66336 | 3576 | I.S. | 0.66336 |
| 3577 | LRP1B | 0.66334 | 3578 | KMT2C | 0.66333 | 3579 | I.S. | 0.66331 |
| 3580 | I.S. | 0.66331 | 3581 | FANCG | 0.6633 | 3582 | I.S. | 0.66328 |
| 3583 | DOT1L | 0.66325 | 3584 | EXO1 | 0.66325 | 3585 | CEBPA | 0.66325 |
| 3586 | I.S. | 0.66322 | 3587 | DNM2 | 0.66322 | 3588 | SPTA1 | 0.6632 |
| 3589 | I.S. | 0.6632 | 3590 | I.S. | 0.6632 | 3591 | I.S. | 0.6632 |
| 3592 | I.S. | 0.6632 | 3593 | I.S. | 0.6632 | 3594 | I.S. | 0.66316 |
| 3595 | MYC | 0.66316 | 3596 | I.S. | 0.66314 | 3597 | I.S. | 0.66309 |
| 3598 | FLT1 | 0.66309 | 3599 | AK2 | 0.66307 | 3600 | I.S. | 0.66306 |
| 3601 | I.S. | 0.66305 | 3602 | PLCG2 | 0.66304 | 3603 | RICTOR | 0.663 |
| 3604 | KMT2C | 0.663 | 3605 | I.S. | 0.663 | 3606 | ALK | 0.66298 |
| 3607 | I.S. | 0.66298 | 3608 | I.S. | 0.66298 | 3609 | I.S. | 0.66298 |
| 3610 | I.S. | 0.66298 | 3611 | INPP4B | 0.66294 | 3612 | I.S. | 0.66287 |
| 3613 | I.S. | 0.66286 | 3614 | I.S. | 0.66286 | 3615 | I.S. | 0.66285 |
| 3616 | I.S. | 0.66284 | 3617 | BMPR1A | 0.66282 | 3618 | I.S. | 0.66281 |
| 3619 | I.S. | 0.66281 | 3620 | I.S. | 0.66281 | 3621 | I.S. | 0.66281 |
| 3622 | I.S. | 0.66281 | 3623 | I.S. | 0.66279 | 3624 | FANCG | 0.66278 |
| 3625 | I.S. | 0.66277 | 3626 | I.S. | 0.66275 | 3627 | I.S. | 0.66273 |
| 3628 | I.S. | 0.66273 | 3629 | CTNNA1 | 0.66272 | 3630 | FLT3 | 0.66271 |
| 3631 | GID4 | 0.66271 | 3632 | NTRK1 | 0.66265 | 3633 | EXO1 | 0.66265 |
| 3634 | DNMT3A | 0.66265 | 3635 | I.S. | 0.66265 | 3636 | I.S. | 0.66265 |
| 3637 | I.S. | 0.66263 | 3638 | FANCM | 0.66263 | 3639 | SUFU | 0.66259 |
| 3640 | I.S. | 0.66259 | 3641 | DNMT3A | 0.66257 | 3642 | I.S. | 0.66253 |
| 3643 | I.S. | 0.66249 | 3644 | I.S. | 0.66249 | 3645 | I.S. | 0.66249 |
| 3646 | I.S. | 0.66249 | 3647 | FGFR2 | 0.66248 | 3648 | RPTOR | 0.66247 |
| 3649 | NFE2L2 | 0.66245 | 3650 | I.S. | 0.66243 | 3651 | I.S. | 0.66242 |
| 3652 | I.S. | 0.66241 | 3653 | PRDM1 | 0.66239 | 3654 | I.S. | 0.66232 |
| 3655 | I.S. | 0.66232 | 3656 | ACD | 0.6623 | 3657 | FLT3 | 0.66227 |
| 3658 | PRDM1 | 0.66227 | 3659 | I.S. | 0.66227 | 3660 | I.S. | 0.66224 |
| 3661 | LYST | 0.66224 | 3662 | PRKN | 0.66223 | 3663 | PBRM1 | 0.66223 |
| 3664 | SLIT2 | 0.66223 | 3665 | I.S. | 0.66222 | 3666 | FLT4 | 0.66221 |
| 3667 | FANCM | 0.66218 | 3668 | MEF2B | 0.66218 | 3669 | DDR2 | 0.66215 |
| 3670 | I.S. | 0.66213 | 3671 | I.S. | 0.6621 | 3672 | I.S. | 0.66209 |
| 3673 | ACD | 0.66209 | 3674 | I.S. | 0.66205 | 3675 | I.S. | 0.66205 |
| 3676 | ARID1A | 0.66203 | 3677 | POLE | 0.66202 | 3678 | I.S. | 0.66202 |
| 3679 | I.S. | 0.66202 | 3680 | I.S. | 0.66202 | 3681 | I.S. | 0.66201 |
| 3682 | I.S. | 0.662 | 3683 | EPHB1 | 0.662 | 3684 | BRIP1 | 0.662 |
| 3685 | I.S. | 0.66196 | 3686 | I.S. | 0.66196 | 3687 | I.S. | 0.66194 |
| 3688 | I.S. | 0.66194 | 3689 | ERBB3 | 0.66194 | 3690 | I.S. | 0.66194 |
| 3691 | I.S. | 0.66194 | 3692 | CTNNA1 | 0.66194 | 3693 | I.S. | 0.66194 |
| 3694 | I.S. | 0.66193 | 3695 | EXO1 | 0.66191 | 3696 | I.S. | 0.6619 |
| 3697 | I.S. | 0.6619 | 3698 | I.S. | 0.66186 | 3699 | I.S. | 0.66186 |
| 3700 | I.S. | 0.66185 | 3701 | I.S. | 0.66183 | 3702 | I.S. | 0.66183 |
| 3703 | I.S. | 0.66182 | 3704 | I.S. | 0.66182 | 3705 | I.S. | 0.66182 |
| 3706 | I.S. | 0.66182 | 3707 | I.S. | 0.66182 | 3708 | I.S. | 0.66181 |
| 3709 | LZTR1 | 0.66174 | 3710 | I.S. | 0.66173 | 3711 | MEFV | 0.66171 |
| 3712 | CHD4 | 0.66171 | 3713 | I.S. | 0.66169 | 3714 | I.S. | 0.66169 |
| 3715 | I.S. | 0.66169 | 3716 | SLIT2 | 0.66168 | 3717 | I.S. | 0.66165 |
| 3718 | TSHR | 0.66163 | 3719 | I.S. | 0.66161 | 3720 | KIF23 | 0.66161 |
| 3721 | FLT4 | 0.66158 | 3722 | SLX4 | 0.66155 | 3723 | FGFR3 | 0.66153 |
| 3724 | I.S. | 0.66151 | 3725 | — | 0.66149 | 3726 | FANCE | 0.66149 |
| 3727 | SRC | 0.66149 | 3728 | I.S. | 0.66148 | 3729 | CIC | 0.66147 |
| 3730 | AR | 0.66147 | 3731 | I.S. | 0.66145 | 3732 | MCL1 | 0.66141 |
| 3733 | DDX41 | 0.66141 | 3734 | I.S. | 0.66139 | 3735 | I.S. | 0.66137 |
| 3736 | KMT2A | 0.66132 | 3737 | CHD4 | 0.66125 | 3738 | I.S. | 0.66125 |
| 3739 | I.S. | 0.6612 | 3740 | I.S. | 0.66117 | 3741 | I.S. | 0.66117 |
| 3742 | I.S. | 0.66114 | 3743 | DAXX | 0.66105 | 3744 | I.S. | 0.66105 |
| 3745 | WDR20 | 0.66105 | 3746 | I.S. | 0.66104 | 3747 | I.S. | 0.66097 |
| 3748 | I.S. | 0.66096 | 3749 | KMT2C | 0.66096 | 3750 | TNFAIP3 | 0.66093 |
| 3751 | FH | 0.66093 | 3752 | I.S. | 0.66092 | 3753 | I.S. | 0.6609 |
| 3754 | RIT1 | 0.66084 | 3755 | I.S. | 0.66082 | 3756 | IKBKE | 0.66082 |
| 3757 | PSTPIP1 | 0.66078 | 3758 | I.S. | 0.66078 | 3759 | I.S. | 0.66076 |
| 3760 | I.S. | 0.66076 | 3761 | FANCD2 | 0.66075 | 3762 | TSC2 | 0.66072 |
| 3763 | I.S. | 0.6607 | 3764 | I.S. | 0.66066 | 3765 | I.S. | 0.66066 |
| 3766 | GLI1 | 0.66066 | 3767 | I.S. | 0.66063 | 3768 | INPP4B | 0.66063 |
| 3769 | ABL1 | 0.66063 | 3770 | ARID1B | 0.6606 | 3771 | I.S. | 0.66057 |
| 3772 | PRDM1 | 0.66057 | 3773 | ERBB2 | 0.66057 | 3774 | I.S. | 0.66054 |
| 3775 | I.S. | 0.66051 | 3776 | I.S. | 0.66049 | 3777 | RUNX1 | 0.66046 |
| 3778 | PDGFRA | 0.66042 | 3779 | SBF2 | 0.6604 | 3780 | I.S. | 0.66035 |
| 3781 | I.S. | 0.66035 | 3782 | I.S. | 0.66034 | 3783 | ARID1B | 0.66034 |
| 3784 | SRSF2, | 0.66034 | 3785 | I.S. | 0.6603 | 3786 | I.S. | 0.66027 |
| | MFSD11 | | | | | | | |
| 3787 | BRCA2 | 0.66027 | 3788 | SLIT2 | 0.66027 | 3789 | PDGFRB | 0.66025 |
| 3790 | CUX1 | 0.66025 | 3791 | I.S. | 0.66023 | 3792 | SEC23B | 0.66019 |
| 3793 | KMT2A | 0.66016 | 3794 | PBRM1 | 0.66013 | 3795 | I.S. | 0.66011 |
| 3796 | I.S. | 0.66011 | 3797 | I.S. | 0.66011 | 3798 | ABL1 | 0.66008 |
| 3799 | I.S. | 0.66007 | 3800 | I.S. | 0.66006 | 3801 | I.S. | 0.66002 |
| 3802 | I.S. | 0.66002 | 3803 | FUBP1 | 0.66001 | 3804 | ARID1B | 0.66001 |
| 3805 | STAT6 | 0.66001 | 3806 | GNA13 | 0.66001 | 3807 | I.S. | 0.65999 |
| 3808 | I.S. | 0.65999 | 3809 | I.S. | 0.65999 | 3810 | I.S. | 0.65999 |
| 3811 | I.S. | 0.65999 | 3812 | LYST | 0.65998 | 3813 | MTOR | 0.65998 |
| 3814 | NSD2 | 0.65995 | 3815 | I.S. | 0.65988 | 3816 | KMT2C | 0.65983 |
| 3817 | DDR2 | 0.65983 | 3818 | RICTOR | 0.65983 | 3819 | SUFU | 0.6598 |
| 3820 | I.S. | 0.6598 | 3821 | I.S. | 0.65978 | 3822 | I.S. | 0.65978 |
| 3823 | RUNX1 | 0.65977 | 3824 | EGFR | 0.65977 | 3825 | POLE | 0.65977 |
| 3826 | ACD | 0.65975 | 3827 | I.S. | 0.65972 | 3828 | I.S. | 0.65972 |
| 3829 | RTEL1 | 0.65972 | 3830 | VEGFA | 0.65968 | 3831 | I.S. | 0.65967 |
| 3832 | I.S. | 0.65966 | 3833 | I.S. | 0.65964 | 3834 | HNF1A | 0.65961 |
| 3835 | I.S. | 0.6596 | 3836 | I.S. | 0.65956 | 3837 | I.S. | 0.65956 |
| 3838 | I.S. | 0.65956 | 3839 | I.S. | 0.65954 | 3840 | I.S. | 0.65951 |
| 3841 | I.S. | 0.6595 | 3842 | NUP93 | 0.6595 | 3843 | BLM | 0.65947 |
| 3844 | I.S. | 0.65945 | 3845 | GABRA6 | 0.65944 | 3846 | FGFR1 | 0.65944 |
| 3847 | NUP93 | 0.65942 | 3848 | SPTA1 | 0.65939 | 3849 | FANCI | 0.65933 |
| 3850 | I.S. | 0.65931 | 3851 | I.S. | 0.65931 | 3852 | I.S. | 0.65931 |
| 3853 | RPTOR | 0.6593 | 3854 | KMT2D | 0.65929 | 3855 | FANCI | 0.65929 |
| 3856 | I.S. | 0.65927 | 3857 | ZNF703 | 0.65927 | 3858 | I.S. | 0.65924 |
| 3859 | RANBP2 | 0.65923 | 3860 | I.S. | 0.65923 | 3861 | I.S. | 0.65923 |
| 3862 | SOX2-OT, | 0.65923 | 3863 | I.S. | 0.65923 | 3864 | FLT1 | 0.65923 |
| | SOX2 | | | | | | | |
| 3865 | PLCG2 | 0.65922 | 3866 | CRKL | 0.65917 | 3867 | I.S. | 0.65915 |
| 3868 | CIC | 0.65912 | 3869 | I.S. | 0.65911 | 3870 | ZNF276, | 0.65911 |
| | | | | | | | FANCA | |
| 3871 | I.S. | 0.65907 | 3872 | DDR2 | 0.65907 | 3873 | SNCAIP | 0.65907 |
| 3874 | EGFR | 0.65906 | 3875 | FGFR3 | 0.65901 | 3876 | I.S. | 0.65899 |
| 3877 | I.S. | 0.65896 | 3878 | I.S. | 0.65895 | 3879 | I.S. | 0.65895 |
| 3880 | I.S. | 0.65893 | 3881 | I.S. | 0.65887 | 3882 | PIK3R1 | 0.65886 |
| 3883 | I.S. | 0.65885 | 3884 | KDR | 0.65879 | 3885 | I.S. | 0.65879 |
| 3886 | I.S. | 0.65873 | 3887 | I.S. | 0.65869 | 3888 | I.S. | 0.65868 |
| 3889 | BMPR1A | 0.65867 | 3890 | EP300 | 0.65867 | 3891 | I.S. | 0.65865 |
| 3892 | I.S. | 0.65862 | 3893 | NOTCH1 | 0.65861 | 3894 | I.S. | 0.65852 |
| 3895 | I.S. | 0.65852 | 3896 | SDHD | 0.65835 | 3897 | I.S. | 0.65833 |
| 3898 | FGFR3 | 0.65832 | 3899 | DICER1 | 0.65832 | 3900 | I.S. | 0.6583 |
| 3901 | I.S. | 0.6583 | 3902 | I.S. | 0.65828 | 3903 | I.S. | 0.65828 |
| 3904 | I.S. | 0.65827 | 3905 | FGFR3 | 0.65826 | 3906 | SDHA | 0.65825 |
| 3907 | I.S. | 0.65825 | 3908 | I.S. | 0.65821 | 3909 | I.S. | 0.6582 |
| 3910 | I.S. | 0.65819 | 3911 | TOP2A | 0.65819 | 3912 | I.S. | 0.65818 |
| 3913 | NTRK2 | 0.65817 | 3914 | MDM2 | 0.65816 | 3915 | RTEL1 | 0.65814 |
| 3916 | I.S. | 0.65811 | 3917 | JAK1 | 0.65811 | 3918 | I.S. | 0.658 |
| 3919 | PIK3R2 | 0.65799 | 3920 | RTEL1 | 0.65798 | 3921 | I.S. | 0.65797 |
| 3922 | I.S. | 0.65797 | 3923 | I.S. | 0.65795 | 3924 | I.S. | 0.65795 |
| 3925 | SRC | 0.65791 | 3926 | TET2 | 0.6579 | 3927 | I.S. | 0.65787 |
| 3928 | I.S. | 0.65787 | 3929 | PLCG2 | 0.65785 | 3930 | KMT2A | 0.65784 |
| 3931 | I.S. | 0.65784 | 3932 | RTEL1 | 0.65784 | 3933 | I.S. | 0.65783 |
| 3934 | CDC73 | 0.65781 | 3935 | I.S. | 0.65778 | 3936 | I.S. | 0.65778 |
| 3937 | ERCC4 | 0.65775 | 3938 | ERBB3 | 0.65775 | 3939 | ROS1 | 0.65774 |
| 3940 | KDR | 0.65772 | 3941 | PIK3C2B | 0.65769 | 3942 | I.S. | 0.65769 |
| 3943 | CDH1 | 0.65769 | 3944 | I.S. | 0.65768 | 3945 | FANCE | 0.65767 |
| 3946 | I.S. | 0.65764 | 3947 | ADGRA2 | 0.65763 | 3948 | EGFR | 0.65762 |
| 3949 | I.S. | 0.65761 | 3950 | I.S. | 0.65761 | 3951 | CHD4 | 0.6576 |
| 3952 | ROS1 | 0.6576 | 3953 | TERF2 | 0.65758 | 3954 | JAK1 | 0.65758 |
| 3955 | FANCD2 | 0.65758 | 3956 | I.S. | 0.65757 | 3957 | I.S. | 0.65751 |
| 3958 | I.S. | 0.6575 | 3959 | SMARCB1 | 0.65749 | 3960 | I.S. | 0.65745 |
| 3961 | I.S. | 0.65745 | 3962 | I.S. | 0.65742 | 3963 | DAXX | 0.65733 |
| 3964 | I.S. | 0.65729 | 3965 | I.S. | 0.65729 | 3966 | I.S. | 0.65728 |
| 3967 | I.S. | 0.65726 | 3968 | SMC3 | 0.65725 | 3969 | I.S. | 0.65722 |
| 3970 | I.S. | 0.6572 | 3971 | KDM5A | 0.65718 | 3972 | I.S. | 0.65714 |
| 3973 | I.S. | 0.65713 | 3974 | I.S. | 0.65712 | 3975 | EPHB1 | 0.6571 |
| 3976 | FANCI | 0.6571 | 3977 | I.S. | 0.65709 | 3978 | I.S. | 0.65706 |
| 3979 | I.S. | 0.65704 | 3980 | I.S. | 0.65704 | 3981 | I.S. | 0.65704 |
| 3982 | PSTPIP1 | 0.65704 | 3983 | I.S. | 0.65704 | 3984 | I.S. | 0.65702 |
| 3985 | EMSY | 0.657 | 3986 | I.S. | 0.65699 | 3987 | GLI1 | 0.65698 |
| 3988 | PDGFRB | 0.65695 | 3989 | PIK3C2B | 0.65695 | 3990 | I.S. | 0.65693 |
| 3991 | I.S. | 0.65693 | 3992 | I.S. | 0.65693 | 3993 | I.S. | 0.65692 |
| 3994 | I.S. | 0.65692 | 3995 | CTCF | 0.65692 | 3996 | NLRP3 | 0.6569 |
| 3997 | I.S. | 0.65687 | 3998 | I.S. | 0.65685 | 3999 | I.S. | 0.65682 |
| 4000 | I.S. | 0.65682 | 4001 | SLIT2 | 0.6568 | 4002 | I.S. | 0.65679 |
| 4003 | MLH1 | 0.65679 | 4004 | FANCA | 0.65679 | 4005 | I.S. | 0.65678 |
| 4006 | EPHB1 | 0.65671 | 4007 | PREX2 | 0.65669 | 4008 | I.S. | 0.65669 |
| 4009 | I.S. | 0.65669 | 4010 | I.S. | 0.65668 | 4011 | FGFR4 | 0.65668 |
| 4012 | ATM | 0.65668 | 4013 | FANCL | 0.65665 | 4014 | I.S. | 0.65662 |
| 4015 | I.S. | 0.65661 | 4016 | I.S. | 0.65661 | 4017 | I.S. | 0.65661 |
| 4018 | I.S. | 0.65661 | 4019 | I.S. | 0.65661 | 4020 | PIM1 | 0.6566 |
| 4021 | ARID2 | 0.65659 | 4022 | EGFR | 0.65659 | 4023 | GRIN2A | 0.65659 |
| 4024 | I.S. | 0.65658 | 4025 | I.S. | 0.65657 | 4026 | I.S. | 0.65657 |
| 4027 | CUL3 | 0.65657 | 4028 | I.S. | 0.65656 | 4029 | I.S. | 0.65654 |
| 4030 | I.S. | 0.65645 | 4031 | I.S. | 0.65638 | 4032 | I.S. | 0.65636 |
| 4033 | TP53 | 0.65636 | 4034 | I.S. | 0.65635 | 4035 | I.S. | 0.65633 |
| 4036 | I.S. | 0.65633 | 4037 | IKBKE | 0.65633 | 4038 | CSF1R | 0.65633 |
| 4039 | KDR | 0.65632 | 4040 | I.S. | 0.65629 | 4041 | I.S. | 0.65628 |
| 4042 | KMT2C | 0.65628 | 4043 | FH | 0.65627 | 4044 | FH | 0.65624 |
| 4045 | I.S. | 0.65622 | 4046 | I.S. | 0.65621 | 4047 | ERBB4 | 0.65617 |
| 4048 | KMT2D | 0.65617 | 4049 | JAK3 | 0.65615 | 4050 | I.S. | 0.65614 |
| 4051 | I.S. | 0.65614 | 4052 | I.S. | 0.65614 | 4053 | I.S. | 0.65614 |
| 4054 | I.S. | 0.65614 | 4055 | I.S. | 0.65614 | 4056 | I.S. | 0.65614 |
| 4057 | ARID1B | 0.65609 | 4058 | FANCM | 0.65606 | 4059 | KAT6A | 0.65597 |
| 4060 | I.S. | 0.65595 | 4061 | DAXX | 0.65594 | 4062 | I.S. | 0.65594 |
| 4063 | I.S. | 0.65592 | 4064 | I.S. | 0.65592 | 4065 | CHD4 | 0.6559 |
| 4066 | I.S. | 0.65589 | 4067 | I.S. | 0.65587 | 4068 | RICTOR | 0.65584 |
| 4069 | I.S. | 0.65583 | 4070 | I.S. | 0.65581 | 4071 | I.S. | 0.65581 |
| 4072 | I.S. | 0.65581 | 4073 | I.S. | 0.65581 | 4074 | TET2 | 0.65579 |
| 4075 | DDX11 | 0.65573 | 4076 | GLI2 | 0.65573 | 4077 | INSD1 | 0.65572 |
| 4078 | I.S. | 0.6557 | 4079 | I.S. | 0.65568 | 4080 | FLT4 | 0.65561 |
| 4081 | I.S. | 0.65561 | 4082 | I.S. | 0.65561 | 4083 | I.S. | 0.65561 |
| 4084 | CTC1 | 0.65559 | 4085 | NSD2 | 0.65556 | 4086 | POLE | 0.65556 |
| 4087 | I.S. | 0.65553 | 4088 | PDGFRA | 0.65553 | 4089 | EMSY | 0.65552 |
| 4090 | KMT2B | 0.65552 | 4091 | I.S. | 0.6555 | 4092 | I.S. | 0.65547 |
| 4093 | I.S. | 0.65547 | 4094 | I.S. | 0.65545 | 4095 | NSD2 | 0.65544 |
| 4096 | SDHD | 0.65544 | 4097 | RPTOR | 0.65544 | 4098 | I.S. | 0.6554 |
| 4099 | I.S. | 0.65537 | 4100 | I.S. | 0.65537 | 4101 | DNM2 | 0.65532 |
| 4102 | I.S. | 0.65531 | 4103 | GALNT12 | 0.65529 | 4104 | I.S. | 0.65528 |
| 4105 | TOP2A | 0.65526 | 4106 | BRD4 | 0.65526 | 4107 | NBN | 0.65525 |
| 4108 | EPHA7 | 0.65525 | 4109 | MET | 0.65525 | 4110 | RAD54L | 0.65523 |
| 4111 | NSD1 | 0.65523 | 4112 | I.S. | 0.6552 | 4113 | I.S. | 0.65519 |
| 4114 | I.S. | 0.65518 | 4115 | I.S. | 0.65517 | 4116 | EPHB1 | 0.65517 |
| 4117 | I.S. | 0.6551 | 4118 | FUBP1 | 0.65508 | 4119 | FANCC | 0.65507 |
| 4120 | I.S. | 0.65505 | 4121 | I.S. | 0.65505 | 4122 | I.S. | 0.65505 |
| 4123 | I.S. | 0.65504 | 4124 | IKZF3 | 0.65504 | 4125 | RUNX1 | 0.65503 |
| 4126 | I.S. | 0.65502 | 4127 | FAT1 | 0.65502 | 4128 | I.S. | 0.65502 |
| 4129 | I.S. | 0.65501 | 4130 | I.S. | 0.65498 | 4131 | KLHL6 | 0.65496 |
| 4132 | I.S. | 0.65496 | 4133 | I.S. | 0.65494 | 4134 | KMT2D | 0.6549 |
| 4135 | PBRM1 | 0.6549 | 4136 | TERF2IP | 0.6549 | 4137 | I.S. | 0.65488 |
| 4138 | RAD51B | 0.65487 | 4139 | FGFR1 | 0.65487 | 4140 | CTNNA1 | 0.65486 |
| 4141 | I.S. | 0.6548 | 4142 | I.S. | 0.6548 | 4143 | I.S. | 0.6548 |
| 4144 | I.S. | 0.6548 | 4145 | I.S. | 0.6548 | 4146 | I.S. | 0.6548 |
| 4147 | I.S. | 0.6548 | 4148 | I.S. | 0.65479 | 4149 | I.S. | 0.65478 |
| 4150 | I.S. | 0.65476 | 4151 | I.S. | 0.65475 | 4152 | I.S. | 0.65475 |
| 4153 | SYK | 0.65471 | 4154 | I.S. | 0.65471 | 4155 | I.S. | 0.65469 |
| 4156 | I.S. | 0.65467 | 4157 | I.S. | 0.65466 | 4158 | DICER1 | 0.65463 |
| 4159 | AXL | 0.65463 | 4160 | NSD1 | 0.6546 | 4161 | I.S. | 0.65455 |
| 4162 | I.S. | 0.65455 | 4163 | I.S. | 0.65455 | 4164 | I.S. | 0.65455 |
| 4165 | I.S. | 0.65455 | 4166 | I.S. | 0.65455 | 4167 | ABL2 | 0.65455 |
| 4168 | FGF14 | 0.65454 | 4169 | KMT2B | 0.65452 | 4170 | ANKRD26 | 0.65449 |
| 4171 | I.S. | 0.65447 | 4172 | PDGFRA | 0.65442 | 4173 | SDHA | 0.65442 |
| 4174 | I.S. | 0.65441 | 4175 | DICER1 | 0.65437 | 4176 | I.S. | 0.65434 |
| 4177 | I.S. | 0.65433 | 4178 | I.S. | 0.65433 | 4179 | I.S. | 0.65433 |
| 4180 | I.S. | 0.65422 | 4181 | GALNT12 | 0.65421 | 4182 | KMT2D | 0.65421 |
| 4183 | I.S. | 0.65421 | 4184 | SEC23B | 0.65419 | 4185 | GFI1 | 0.65418 |
| 4186 | CREBBP | 0.65414 | 4187 | I.S. | 0.65411 | 4188 | I.S. | 0.65408 |
| 4189 | I.S. | 0.65404 | 4190 | FLT3 | 0.65404 | 4191 | AKT3 | 0.65401 |
| 4192 | I.S. | 0.654 | 4193 | I.S. | 0.654 | 4194 | I.S. | 0.654 |
| 4195 | I.S. | 0.654 | 4196 | I.S. | 0.654 | 4197 | I.S. | 0.65398 |
| 4198 | GREM1 | 0.65396 | 4199 | IRF4 | 0.65394 | 4200 | I.S. | 0.65392 |
| 4201 | I.S. | 0.65392 | 4202 | I.S. | 0.6539 | 4203 | KDM5A | 0.65385 |
| 4204 | — | 0.65384 | 4205 | I.S. | 0.65383 | 4206 | FLT1 | 0.65383 |
| 4207 | PMS2 | 0.6538 | 4208 | I.S. | 0.6538 | 4209 | I.S. | 0.6538 |
| 4210 | I.S. | 0.65378 | 4211 | I.S. | 0.65376 | 4212 | I.S. | 0.65375 |
| 4213 | I.S. | 0.65372 | 4214 | I.S. | 0.65371 | 4215 | — | 0.65371 |
| 4216 | MAP3K14 | 0.65368 | 4217 | BARD1 | 0.65368 | 4218 | SPEN | 0.65367 |
| 4219 | I.S. | 0.65367 | 4220 | I.S. | 0.65367 | 4221 | I.S. | 0.65362 |
| 4222 | I.S. | 0.6536 | 4223 | ADA | 0.65359 | 4224 | I.S. | 0.65357 |
| 4225 | TSHR | 0.65356 | 4226 | I.S. | 0.65354 | 4227 | I.S. | 0.65353 |
| 4228 | I.S. | 0.65351 | 4229 | I.S. | 0.65351 | 4230 | POLE | 0.65347 |
| 4231 | IKBKE | 0.65345 | 4232 | FLT4 | 0.65344 | 4233 | I.S. | 0.6534 |
| 4234 | I.S. | 0.65337 | 4235 | I.S. | 0.65337 | 4236 | NF1 | 0.65335 |
| 4237 | I.S. | 0.6533 | 4238 | I.S. | 0.65327 | 4239 | I.S. | 0.65327 |
| 4240 | EMSY | 0.65323 | 4241 | I.S. | 0.65321 | 4242 | I.S. | 0.65319 |
| 4243 | I.S. | 0.65319 | 4244 | PAX5 | 0.65317 | 4245 | BRCA2 | 0.65314 |
| 4246 | I.S. | 0.65312 | 4247 | I.S. | 0.65307 | 4248 | AK2 | 0.65306 |
| 4249 | SRC | 0.65302 | 4250 | I.S. | 0.65301 | 4251 | I.S. | 0.653 |
| 4252 | I.S. | 0.65299 | 4253 | I.S. | 0.65296 | 4254 | I.S. | 0.65296 |
| 4255 | PIK3R2 | 0.65291 | 4256 | I.S. | 0.65288 | 4257 | BRAF | 0.65285 |
| 4258 | I.S. | 0.65285 | 4259 | I.S. | 0.6528 | 4260 | SDHA | 0.65279 |
| 4261 | I.S. | 0.65278 | 4262 | ARID1B | 0.65276 | 4263 | I.S. | 0.65274 |
| 4264 | GLI2 | 0.65273 | 4265 | I.S. | 0.65272 | 4266 | I.S. | 0.65272 |
| 4267 | I.S. | 0.65272 | 4268 | I.S. | 0.65272 | 4269 | I.S. | 0.65272 |
| 4270 | I.S. | 0.65272 | 4271 | I.S. | 0.65272 | 4272 | CDC73 | 0.65266 |
| 4273 | SLX4 | 0.65265 | 4274 | NF1 | 0.65264 | 4275 | FAT1 | 0.65262 |
| 4276 | I.S. | 0.6526 | 4277 | I.S. | 0.6526 | 4278 | I.S. | 0.6526 |
| 4279 | PRKAR1A | 0.65259 | 4280 | TCIRG1 | 0.65258 | 4281 | POLR2F, | 0.65255 |
| | | | | | | | SOX10 | |
| 4282 | I.S. | 0.65253 | 4283 | I.S. | 0.65252 | 4284 | EPHA3 | 0.65247 |
| 4285 | LYST | 0.65247 | 4286 | I.S. | 0.65241 | 4287 | I.S. | 0.65241 |
| 4288 | I.S. | 0.65241 | 4289 | I.S. | 0.65241 | 4290 | I.S. | 0.65241 |
| 4291 | I.S. | 0.65239 | 4292 | I.S. | 0.65239 | 4293 | I.S. | 0.65233 |
| 4294 | I.S. | 0.6523 | 4295 | I.S. | 0.65228 | 4296 | I.S. | 0.65226 |
| 4297 | I.S. | 0.65223 | 4298 | PLCG1 | 0.6522 | 4299 | I.S. | 0.65219 |
| 4300 | I.S. | 0.65213 | 4301 | CDKN1A, | 0.65211 | 4302 | VEGFA | 0.65211 |
| | | | | DINOL | | | | |
| 4303 | I.S. | 0.65211 | 4304 | I.S. | 0.65209 | 4305 | I.S. | 0.65208 |
| 4306 | IKZF1 | 0.65208 | 4307 | GNAS | 0.65208 | 4308 | I.S. | 0.65206 |
| 4309 | PIK3C2B | 0.65205 | 4310 | I.S. | 0.65204 | 4311 | I.S. | 0.65203 |
| 4312 | I.S. | 0.65202 | 4313 | I.S. | 0.65202 | 4314 | I.S. | 0.65201 |
| 4315 | I.S. | 0.65199 | 4316 | I.S. | 0.65197 | 4317 | RICTOR | 0.65195 |
| 4318 | I.S. | 0.65195 | 4319 | FGFR4 | 0.65194 | 4320 | I.S. | 0.6519 |
| 4321 | SMC3 | 0.6519 | 4322 | RNF168 | 0.65189 | 4323 | LRP1B | 0.65187 |
| 4324 | KDM5A | 0.65187 | 4325 | I.S. | 0.65186 | 4326 | I.S. | 0.65184 |
| 4327 | I.S. | 0.65184 | 4328 | I.S. | 0.65182 | 4329 | I.S. | 0.65181 |
| 4330 | I.S. | 0.65175 | 4331 | KMT2C | 0.65171 | 4332 | I.S. | 0.65171 |
| 4333 | I.S. | 0.6517 | 4334 | ERG | 0.65166 | 4335 | I.S. | 0.65165 |
| 4336 | I.S. | 0.65164 | 4337 | CDK12 | 0.65163 | 4338 | MLH1 | 0.65163 |
| 4339 | I.S. | 0.65163 | 4340 | I.S. | 0.65162 | 4341 | I.S. | 0.65162 |
| 4342 | I.S. | 0.65162 | 4343 | I.S. | 0.65162 | 4344 | — | 0.65161 |
| 4345 | I.S. | 0.65154 | 4346 | I.S. | 0.65154 | 4347 | CREBBP | 0.65152 |
| 4348 | I.S. | 0.65146 | 4349 | I.S. | 0.65145 | 4350 | LRP1B | 0.65145 |
| 4351 | PIK3C2B | 0.65143 | 4352 | I.S. | 0.65142 | 4353 | PIK3R2 | 0.65142 |
| 4354 | I.S. | 0.65138 | 4355 | I.S. | 0.65138 | 4356 | I.S. | 0.65138 |
| 4357 | I.S. | 0.65138 | 4358 | I.S. | 0.65137 | 4359 | I.S. | 0.65136 |
| 4360 | TSC2 | 0.65136 | 4361 | I.S. | 0.65134 | 4362 | I.S. | 0.65134 |
| 4363 | STAT3 | 0.65133 | 4364 | VEGFA | 0.65128 | 4365 | I.S. | 0.65128 |
| 4366 | PIK3C2B | 0.65127 | 4367 | ARID2 | 0.65127 | 4368 | I.S. | 0.65126 |
| 4369 | IKBKE | 0.65119 | 4370 | I.S. | 0.65116 | 4371 | I.S. | 0.65115 |
| 4372 | I.S. | 0.65113 | 4373 | I.S. | 0.6511 | 4374 | JAK2 | 0.6511 |
| 4375 | VEGFA | 0.65108 | 4376 | SOX9 | 0.65107 | 4377 | I.S. | 0.65105 |
| 4378 | I.S. | 0.65105 | 4379 | I.S. | 0.65105 | 4380 | I.S. | 0.65105 |
| 4381 | I.S. | 0.65105 | 4382 | I.S. | 0.65105 | 4383 | PIK3C2B | 0.65104 |
| 4384 | CBLC | 0.65104 | 4385 | I.S. | 0.65103 | 4386 | I.S. | 0.65102 |
| 4387 | I.S. | 0.65102 | 4388 | I.S. | 0.651 | 4389 | RICTOR | 0.65098 |
| 4390 | I.S. | 0.65097 | 4391 | I.S. | 0.65097 | 4392 | I.S. | 0.65097 |
| 4393 | AXL | 0.65095 | 4394 | I.S. | 0.65095 | 4395 | I.S. | 0.65094 |
| 4396 | I.S. | 0.65091 | 4397 | I.S. | 0.65091 | 4398 | I.S. | 0.65091 |
| 4399 | I.S. | 0.65091 | 4400 | IGF1R | 0.65089 | 4401 | I.S. | 0.65083 |
| 4402 | I.S. | 0.65083 | 4403 | I.S. | 0.65083 | 4404 | CDAN1 | 0.65083 |
| 4405 | I.S. | 0.65081 | 4406 | RAD50 | 0.65079 | 4407 | I.S. | 0.65074 |
| 4408 | I.S. | 0.65072 | 4409 | SMC3 | 0.65071 | 4410 | RAD51B | 0.65071 |
| 4411 | I.S. | 0.6507 | 4412 | I.S. | 0.65069 | 4413 | I.S. | 0.65064 |
| 4414 | I.S. | 0.65063 | 4415 | I.S. | 0.65062 | 4416 | RB1 | 0.65059 |
| 4417 | PTPN11 | 0.65058 | 4418 | I.S. | 0.65058 | 4419 | I.S. | 0.65058 |
| 4420 | I.S. | 0.65058 | 4421 | I.S. | 0.65058 | 4422 | I.S. | 0.65058 |
| 4423 | I.S. | 0.65058 | 4424 | I.S. | 0.65058 | 4425 | I.S. | 0.65058 |
| 4426 | CIC | 0.65057 | 4427 | EPHA3 | 0.6505 | 4428 | I.S. | 0.65048 |
| 4429 | PRKN | 0.65047 | 4430 | I.S. | 0.65047 | 4431 | I.S. | 0.65039 |
| 4432 | I.S. | 0.65039 | 4433 | I.S. | 0.65036 | 4434 | I.S. | 0.65034 |
| 4435 | I.S. | 0.65034 | 4436 | I.S. | 0.65034 | 4437 | I.S. | 0.65031 |
| 4438 | I.S. | 0.6503 | 4439 | I.S. | 0.65028 | 4440 | I.S. | 0.65028 |
| 4441 | I.S. | 0.65027 | 4442 | CSF3R | 0.65027 | 4443 | CHEK1 | 0.65027 |
| 4444 | I.S. | 0.65026 | 4445 | I.S. | 0.65026 | 4446 | I.S. | 0.65026 |
| 4447 | I.S. | 0.65026 | 4448 | I.S. | 0.65026 | 4449 | I.S. | 0.65022 |
| 4450 | ERG | 0.65021 | 4451 | I.S. | 0.65021 | 4452 | I.S. | 0.65018 |
| 4453 | STK11 | 0.65018 | 4454 | I.S. | 0.65017 | 4455 | I.S. | 0.65017 |
| 4456 | FANCG | 0.65014 | 4457 | PRKCI | 0.65011 | 4458 | — | 0.6501 |
| 4459 | JAK3 | 0.65009 | 4460 | BARD1 | 0.65006 | 4461 | CREBBP | 0.65005 |
| 4462 | I.S. | 0.65003 | 4463 | I.S. | 0.65003 | 4464 | I.S. | 0.65002 |
| 4465 | I.S. | 0.65001 | 4466 | I.S. | 0.64998 | 4467 | I.S. | 0.64995 |
| 4468 | ROS1 | 0.64995 | 4469 | LRP1B | 0.64994 | 4470 | I.S. | 0.64993 |
| 4471 | I.S. | 0.6499 | 4472 | I.S. | 0.64989 | 4473 | TET2 | 0.64988 |
| 4474 | MAP2K4 | 0.64987 | 4475 | I.S. | 0.64986 | 4476 | SDHA | 0.64984 |
| 4477 | TSC1 | 0.64984 | 4478 | I.S. | 0.64984 | 4479 | NTRK1 | 0.64982 |
| 4480 | CDK8 | 0.64981 | 4481 | I.S. | 0.64981 | 4482 | I.S. | 0.64979 |
| 4483 | I.S. | 0.64979 | 4484 | I.S. | 0.64979 | 4485 | BRCA2 | 0.64979 |
| 4486 | I.S. | 0.64979 | 4487 | I.S. | 0.64976 | 4488 | LYST | 0.64973 |
| 4489 | TSHR | 0.64973 | 4490 | I.S. | 0.64969 | 4491 | TBX3 | 0.64966 |
| 4492 | I.S. | 0.64964 | 4493 | SLIT2 | 0.64964 | 4494 | I.S. | 0.64961 |
| 4495 | I.S. | 0.64961 | 4496 | KDM5A | 0.6496 | 4497 | I.S. | 0.6496 |
| 4498 | I.S. | 0.6496 | 4499 | I.S. | 0.64959 | 4500 | I.S. | 0.64957 |
| 4501 | I.S. | 0.64955 | 4502 | I.S. | 0.64955 | 4503 | JUN | 0.64952 |
| 4504 | I.S. | 0.64951 | 4505 | I.S. | 0.64948 | 4506 | I.S. | 0.64946 |
| 4507 | I.S. | 0.64945 | 4508 | I.S. | 0.64943 | 4509 | I.S. | 0.64943 |
| 4510 | BRCA1 | 0.64938 | 4511 | SYK | 0.64937 | 4512 | I.S. | 0.64936 |
| 4513 | NOTCH2 | 0.64931 | 4514 | I.S. | 0.64924 | 4515 | I.S. | 0.64924 |
| 4516 | I.S. | 0.64924 | 4517 | I.S. | 0.64924 | 4518 | I.S. | 0.64923 |
| 4519 | DAXX | 0.64923 | 4520 | FGF10 | 0.64922 | 4521 | DNMT3A | 0.6492 |
| 4522 | APC | 0.64919 | 4523 | I.S. | 0.64916 | 4524 | I.S. | 0.64914 |
| 4525 | I.S. | 0.64912 | 4526 | I.S. | 0.6491 | 4527 | GATA4 | 0.6491 |
| 4528 | PDGFRB | 0.64907 | 4529 | IKZF1 | 0.64902 | 4530 | I.S. | 0.64902 |
| 4531 | I.S. | 0.64893 | 4532 | I.S. | 0.64891 | 4533 | I.S. | 0.64891 |
| 4534 | NFKBIA | 0.6489 | 4535 | I.S. | 0.64889 | 4536 | BRCA2 | 0.64889 |
| 4537 | I.S. | 0.64888 | 4538 | I.S. | 0.64888 | 4539 | TSHR | 0.64887 |
| 4540 | I.S. | 0.64881 | 4541 | I.S. | 0.64877 | 4542 | I.S. | 0.64877 |
| 4543 | I.S. | 0.64877 | 4544 | I.S. | 0.64872 | 4545 | I.S. | 0.6487 |
| 4546 | KLHL6 | 0.64869 | 4547 | I.S. | 0.64869 | 4548 | NTRK2 | 0.64868 |
| 4549 | I.S. | 0.64867 | 4550 | I.S. | 0.64867 | 4551 | I.S. | 0.64867 |
| 4552 | I.S. | 0.64865 | 4553 | I.S. | 0.64864 | 4554 | NTRK1 | 0.64863 |
| 4555 | I.S. | 0.64863 | 4556 | I.S. | 0.64861 | 4557 | SDHD | 0.64861 |
| 4558 | I.S. | 0.64861 | 4559 | IRF2 | 0.6486 | 4560 | I.S. | 0.64857 |
| 4561 | I.S. | 0.64855 | 4562 | I.S. | 0.64855 | 4563 | I.S. | 0.64853 |
| 4564 | I.S. | 0.6485 | 4565 | I.S. | 0.64849 | 4566 | MEFV | 0.64848 |
| 4567 | I.S. | 0.64848 | 4568 | I.S. | 0.64847 | 4569 | I.S. | 0.64845 |
| 4570 | I.S. | 0.64845 | 4571 | I.S. | 0.64845 | 4572 | I.S. | 0.64845 |
| 4573 | I.S. | 0.64845 | 4574 | I.S. | 0.64845 | 4575 | I.S. | 0.64845 |
| 4576 | I.S. | 0.64845 | 4577 | I.S. | 0.64845 | 4578 | I.S. | 0.64845 |
| 4579 | I.S. | 0.64841 | 4580 | ERBB3 | 0.64837 | 4581 | I.S. | 0.64836 |
| 4582 | PRKCI | 0.64835 | 4583 | BRD4 | 0.64835 | 4584 | I.S. | 0.64833 |
| 4585 | TBX3 | 0.64833 | 4586 | CTCF | 0.64828 | 4587 | I.S. | 0.64826 |
| 4588 | I.S. | 0.64824 | 4589 | I.S. | 0.64824 | 4590 | I.S. | 0.64822 |
| 4591 | I.S. | 0.64822 | 4592 | SMAD9 | 0.64821 | 4593 | I.S. | 0.64821 |
| 4594 | TOP2A | 0.64821 | 4595 | I.S. | 0.6482 | 4596 | I.S. | 0.6482 |
| 4597 | I.S. | 0.6482 | 4598 | I.S. | 0.64819 | 4599 | TERF2 | 0.64819 |
| 4600 | I.S. | 0.64818 | 4601 | JAK3 | 0.64816 | 4602 | FLT1 | 0.64815 |
| 4603 | I.S. | 0.64814 | 4604 | CDKN1B | 0.64813 | 4605 | I.S. | 0.64812 |
| 4606 | I.S. | 0.64812 | 4607 | MYC | 0.64812 | 4608 | I.S. | 0.64812 |
| 4609 | I.S. | 0.64811 | 4610 | I.S. | 0.64809 | 4611 | ATM | 0.64806 |
| 4612 | CTC1 | 0.64804 | 4613 | I.S. | 0.64801 | 4614 | CSF3R | 0.648 |
| 4615 | SETBP1 | 0.64797 | 4616 | I.S. | 0.64796 | 4617 | I.S. | 0.64796 |
| 4618 | I.S. | 0.64796 | 4619 | SDHB | 0.64795 | 4620 | KMT2D | 0.64795 |
| 4621 | I.S. | 0.64788 | 4622 | I.S. | 0.64788 | 4623 | I.S. | 0.64788 |
| 4624 | I.S. | 0.64785 | 4625 | I.S. | 0.64785 | 4626 | I.S. | 0.64784 |
| 4627 | I.S. | 0.64784 | 4628 | I.S. | 0.64783 | 4629 | FOXP1 | 0.64783 |
| 4630 | I.S. | 0.64781 | 4631 | PMS1 | 0.64774 | 4632 | I.S. | 0.64772 |
| 4633 | I.S. | 0.64757 | 4634 | I.S. | 0.6475 | 4635 | TOP1 | 0.6475 |
| 4636 | FANCI | 0.6475 | 4637 | I.S. | 0.64745 | 4638 | FAT1 | 0.64745 |
| 4639 | I.S. | 0.64744 | 4640 | I.S. | 0.64743 | 4641 | I.S. | 0.64743 |
| 4642 | I.S. | 0.64741 | 4643 | I.S. | 0.64741 | 4644 | I.S. | 0.64741 |
| 4645 | I.S. | 0.64741 | 4646 | I.S. | 0.64741 | 4647 | TOP1 | 0.64739 |
| 4648 | I.S. | 0.64738 | 4649 | I.S. | 0.64732 | 4650 | TRAF3 | 0.64726 |
| 4651 | I.S. | 0.64723 | 4652 | I.S. | 0.64723 | 4653 | AXIN1 | 0.64723 |
| 4654 | I.S. | 0.64722 | 4655 | I.S. | 0.64721 | 4656 | FLCN | 0.64721 |
| 4657 | FAT1 | 0.64718 | 4658 | I.S. | 0.64716 | 4659 | PIK3CA | 0.64716 |
| 4660 | FANCA | 0.64716 | 4661 | I.S. | 0.64715 | 4662 | PLCG2 | 0.64715 |
| 4663 | I.S. | 0.64715 | 4664 | I.S. | 0.64715 | 4665 | CBL | 0.64712 |
| 4666 | I.S. | 0.64712 | 4667 | I.S. | 0.6471 | 4668 | I.S. | 0.6471 |
| 4669 | I.S. | 0.6471 | 4670 | I.S. | 0.64708 | 4671 | I.S. | 0.64708 |
| 4672 | CTNNA1 | 0.64705 | 4673 | I.S. | 0.64705 | 4674 | I.S. | 0.64705 |
| 4675 | I.S. | 0.64705 | 4676 | FLT1 | 0.64702 | 4677 | I.S. | 0.647 |
| 4678 | SLX4 | 0.64697 | 4679 | ARID1A | 0.64696 | 4680 | NSD2 | 0.64696 |
| 4681 | NSD2 | 0.64696 | 4682 | I.S. | 0.64696 | 4683 | I.S. | 0.64694 |
| 4684 | I.S. | 0.64686 | 4685 | I.S. | 0.64686 | 4686 | I.S. | 0.64686 |
| 4687 | I.S. | 0.64684 | 4688 | I.S. | 0.64684 | 4689 | I.S. | 0.64684 |
| 4690 | I.S. | 0.64684 | 4691 | I.S. | 0.64684 | 4692 | I.S. | 0.64679 |
| 4693 | FAT1 | 0.64676 | 4694 | I.S. | 0.64676 | 4695 | FANCF | 0.64676 |
| 4696 | I.S. | 0.64674 | 4697 | I.S. | 0.64674 | 4698 | EXO1 | 0.64674 |
| 4699 | I.S. | 0.64672 | 4700 | I.S. | 0.64672 | 4701 | I.S. | 0.6467 |
| 4702 | I.S. | 0.64667 | 4703 | I.S. | 0.64667 | 4704 | I.S. | 0.64664 |
| 4705 | I.S. | 0.64664 | 4706 | — | 0.64664 | 4707 | ASXL1 | 0.64664 |
| 4708 | FANCM | 0.64659 | 4709 | LRP1B | 0.64655 | 4710 | I.S. | 0.64655 |
| 4711 | I.S. | 0.64653 | 4712 | I.S. | 0.64653 | 4713 | I.S. | 0.6465 |
| 4714 | KMT2C | 0.64647 | 4715 | I.S. | 0.64647 | 4716 | FANCI | 0.64646 |
| 4717 | BRCA2 | 0.64646 | 4718 | I.S. | 0.64645 | 4719 | I.S. | 0.64645 |
| 4720 | I.S. | 0.64645 | 4721 | MSH6 | 0.64644 | 4722 | I.S. | 0.64644 |
| 4723 | I.S. | 0.64643 | 4724 | I.S. | 0.64641 | 4725 | I.S. | 0.64639 |
| 4726 | I.S. | 0.64637 | 4727 | I.S. | 0.64634 | 4728 | KDM5A | 0.64631 |
| 4729 | I.S. | 0.64631 | 4730 | I.S. | 0.64631 | 4731 | I.S. | 0.64631 |
| 4732 | I.S. | 0.64631 | 4733 | I.S. | 0.64631 | 4734 | I.S. | 0.64631 |
| 4735 | I.S. | 0.64631 | 4736 | I.S. | 0.64629 | 4737 | G6PC3 | 0.64628 |
| 4738 | I.S. | 0.64628 | 4739 | I.S. | 0.64619 | 4740 | I.S. | 0.64618 |
| 4741 | I.S. | 0.64618 | 4742 | I.S. | 0.64618 | 4743 | I.S. | 0.64615 |
| 4744 | I.S. | 0.64614 | 4745 | PLCG2 | 0.64614 | 4746 | I.S. | 0.64612 |
| 4747 | BRCA1 | 0.6461 | 4748 | RUNX1T1 | 0.64608 | 4749 | STAT6 | 0.64607 |
| 4750 | I.S. | 0.64603 | 4751 | I.S. | 0.64601 | 4752 | I.S. | 0.64601 |
| 4753 | I.S. | 0.646 | 4754 | I.S. | 0.64598 | 4755 | I.S. | 0.64596 |
| 4756 | VEGFA | 0.64595 | 4757 | I.S. | 0.64595 | 4758 | WT1 | 0.64593 |
| 4759 | BRAF | 0.64593 | 4760 | I.S. | 0.64591 | 4761 | I.S. | 0.6459 |
| 4762 | CBL | 0.6459 | 4763 | I.S. | 0.64589 | 4764 | I.S. | 0.64589 |
| 4765 | I.S. | 0.64587 | 4766 | I.S. | 0.64585 | 4767 | I.S. | 0.64582 |
| 4768 | I.S. | 0.64579 | 4769 | CDAN1 | 0.64576 | 4770 | RNF43 | 0.64573 |
| 4771 | PDGFRA | 0.64573 | 4772 | I.S. | 0.64571 | 4773 | I.S. | 0.6457 |
| 4774 | I.S. | 0.6457 | 4775 | I.S. | 0.64569 | 4776 | PDGFRB | 0.64569 |
| 4777 | I.S. | 0.64558 | 4778 | I.S. | 0.64557 | 4779 | ARID1A | 0.64551 |
| 4780 | I.S. | 0.64549 | 4781 | I.S. | 0.64549 | 4782 | I.S. | 0.64549 |
| 4783 | I.S. | 0.64549 | 4784 | I.S. | 0.64548 | 4785 | FLT1 | 0.64548 |
| 4786 | I.S. | 0.64546 | 4787 | TOP2A | 0.64545 | 4788 | RIT1 | 0.6454 |
| 4789 | CDK12 | 0.64539 | 4790 | I.S. | 0.64535 | 4791 | I.S. | 0.64535 |
| 4792 | I.S. | 0.64534 | 4793 | I.S. | 0.64533 | 4794 | I.S. | 0.64533 |
| 4795 | I.S. | 0.64529 | 4796 | I.S. | 0.64527 | 4797 | I.S. | 0.64527 |
| 4798 | I.S. | 0.64524 | 4799 | I.S. | 0.64524 | 4800 | I.S. | 0.64519 |
| 4801 | I.S. | 0.64518 | 4802 | KMT2A | 0.64516 | 4803 | I.S. | 0.64513 |
| 4804 | I.S. | 0.64513 | 4805 | I.S. | 0.64513 | 4806 | I.S. | 0.64513 |
| 4807 | SLIT2 | 0.64512 | 4808 | I.S. | 0.64511 | 4809 | I.S. | 0.6451 |
| 4810 | SNCAIP | 0.64509 | 4811 | I.S. | 0.64509 | 4812 | I.S. | 0.64508 |
| 4813 | SLX4 | 0.64507 | 4814 | TOP2A | 0.64507 | 4815 | CD274 | 0.64507 |
| 4816 | LRP1B | 0.64503 | 4817 | I.S. | 0.64503 | 4818 | I.S. | 0.64503 |
| 4819 | I.S. | 0.64503 | 4820 | I.S. | 0.64503 | 4821 | I.S. | 0.64503 |
| 4822 | I.S. | 0.64503 | 4823 | I.S. | 0.64503 | 4824 | I.S. | 0.64503 |
| 4825 | I.S. | 0.64503 | 4826 | I.S. | 0.64503 | 4827 | I.S. | 0.64503 |
| 4828 | I.S. | 0.64503 | 4829 | I.S. | 0.64503 | 4830 | I.S. | 0.64503 |
| 4831 | I.S. | 0.64503 | 4832 | I.S. | 0.64503 | 4833 | I.S. | 0.64503 |
| 4834 | I.S. | 0.64503 | 4835 | I.S. | 0.64501 | 4836 | MAGI2 | 0.645 |
| 4837 | GATA2 | 0.645 | 4838 | I.S. | 0.64499 | 4839 | I.S. | 0.64497 |
| 4840 | I.S. | 0.64496 | 4841 | I.S. | 0.64494 | 4842 | I.S. | 0.64494 |
| 4843 | I.S. | 0.64491 | 4844 | I.S. | 0.64491 | 4845 | I.S. | 0.64489 |
| 4846 | I.S. | 0.64489 | 4847 | SPTA1 | 0.64489 | 4848 | EMSY | 0.64486 |
| 4849 | I.S. | 0.6448 | 4850 | FANCE | 0.64479 | 4851 | MAP2K2 | 0.64479 |
| 4852 | I.S. | 0.64479 | 4853 | I.S. | 0.64478 | 4854 | I.S. | 0.64478 |
| 4855 | I.S. | 0.64476 | 4856 | KIT | 0.64475 | 4857 | I.S. | 0.64472 |
| 4858 | I.S. | 0.64472 | 4859 | I.S. | 0.6447 | 4860 | I.S. | 0.6447 |
| 4861 | I.S. | 0.6447 | 4862 | I.S. | 0.6447 | 4863 | ARID1A | 0.64468 |
| 4864 | KMT2B | 0.64468 | 4865 | CDH1 | 0.64465 | 4866 | I.S. | 0.64464 |
| 4867 | I.S. | 0.64461 | 4868 | I.S. | 0.64458 | 4869 | CTNNB1 | 0.64456 |
| 4870 | I.S. | 0.64456 | 4871 | STAT6 | 0.64453 | 4872 | I.S. | 0.6445 |
| 4873 | I.S. | 0.6445 | 4874 | EP300 | 0.64447 | 4875 | I.S. | 0.64444 |
| 4876 | NTRK3 | 0.64443 | 4877 | BLM | 0.64442 | 4878 | I.S. | 0.6444 |
| 4879 | I.S. | 0.6444 | 4880 | DNMT3A | 0.64438 | 4881 | I.S. | 0.64436 |
| 4882 | I.S. | 0.64434 | 4883 | I.S. | 0.64434 | 4884 | KAT6A | 0.64424 |
| 4885 | BRD4 | 0.64421 | 4886 | I.S. | 0.64417 | 4887 | I.S. | 0.64417 |
| 4888 | TCF3 | 0.64417 | 4889 | TSC2 | 0.64415 | 4890 | I.S. | 0.6441 |
| 4891 | — | 0.64409 | 4892 | I.S. | 0.64409 | 4893 | I.S. | 0.64407 |
| 4894 | I.S. | 0.64407 | 4895 | IGF1R | 0.64403 | 4896 | I.S. | 0.64401 |
| 4897 | I.S. | 0.64401 | 4898 | I.S. | 0.644 | 4899 | I.S. | 0.64399 |
| 4900 | I.S. | 0.64399 | 4901 | I.S. | 0.64397 | 4902 | GLI2 | 0.64396 |
| 4903 | LYST | 0.64393 | 4904 | I.S. | 0.64393 | 4905 | | 0.64392 |
| 4906 | ERBB3 | 0.64388 | 4907 | I.S. | 0.64387 | 4908 | I.S. | 0.64387 |
| 4909 | IKBKE | 0.64385 | 4910 | I.S. | 0.64382 | 4911 | STAT6 | 0.64381 |
| 4912 | I.S. | 0.6438 | 4913 | NF1 | 0.6438 | 4914 | CREBBP | 0.64376 |
| 4915 | TSC2 | 0.64373 | 4916 | RAD51B | 0.64373 | 4917 | I.S. | 0.64369 |
| 4918 | I.S. | 0.64368 | 4919 | I.S. | 0.64368 | 4920 | I.S. | 0.64368 |
| 4921 | I.S. | 0.64368 | 4922 | I.S. | 0.64368 | 4923 | I.S. | 0.64368 |
| 4924 | I.S. | 0.64368 | 4925 | I.S. | 0.64363 | 4926 | I.S. | 0.64363 |
| 4927 | I.S. | 0.64363 | 4928 | I.S. | 0.64358 | 4929 | I.S. | 0.64355 |
| 4930 | I.S. | 0.64352 | 4931 | RAD51 | 0.64352 | 4932 | SPTA1 | 0.6435 |
| 4933 | WT1 | 0.64346 | 4934 | I.S. | 0.64344 | 4935 | I.S. | 0.64344 |
| 4936 | I.S. | 0.64344 | 4937 | I.S. | 0.64344 | 4938 | SPEN | 0.64338 |
| 4939 | AURKA | 0.64338 | 4940 | I.S. | 0.64336 | 4941 | I.S. | 0.64336 |
| 4942 | I.S. | 0.64336 | 4943 | I.S. | 0.64336 | 4944 | I.S. | 0.64336 |
| 4945 | I.S. | 0.64336 | 4946 | I.S. | 0.64332 | 4947 | I.S. | 0.64326 |
| 4948 | CDAN1 | 0.64326 | 4949 | I.S. | 0.64325 | 4950 | STAT4 | 0.64322 |
| 4951 | I.S. | 0.64322 | 4952 | I.S. | 0.64322 | 4953 | I.S. | 0.64322 |
| 4954 | HSD3B1 | 0.64319 | 4955 | I.S. | 0.64317 | 4956 | IKBKE | 0.64316 |
| 4957 | I.S. | 0.64313 | 4958 | I.S. | 0.64313 | 4959 | I.S. | 0.64311 |
| 4960 | I.S. | 0.6431 | 4961 | BRCA1 | 0.6431 | 4962 | NOTCH2 | 0.64308 |
| 4963 | KLHL6 | 0.64308 | 4964 | I.S. | 0.64307 | 4965 | KMT2A | 0.64307 |
| 4966 | I.S. | 0.64305 | 4967 | I.S. | 0.64303 | 4968 | I.S. | 0.64301 |
| 4969 | I.S. | 0.643 | 4970 | I.S. | 0.643 | 4971 | — | 0.64299 |
| 4972 | I.S. | 0.64297 | 4973 | KAT6A | 0.64292 | 4974 | I.S. | 0.64291 |
| 4975 | I.S. | 0.64291 | 4976 | I.S. | 0.64291 | 4977 | I.S. | 0.6429 |
| 4978 | I.S. | 0.64289 | 4979 | I.S. | 0.64289 | 4980 | I.S. | 0.64288 |
| 4981 | RBBP6 | 0.64281 | 4982 | CSF1R | 0.64277 | 4983 | I.S. | 0.64275 |
| 4984 | KEL | 0.64275 | 4985 | JAK1 | 0.64274 | 4986 | I.S. | 0.64272 |
| 4987 | I.S. | 0.6427 | 4988 | SDHA | 0.64266 | 4989 | I.S. | 0.64265 |
| 4990 | I.S. | 0.64265 | 4991 | I.S. | 0.64265 | 4992 | G6PC3 | 0.64263 |
| 4993 | I.S. | 0.64259 | 4994 | I.S. | 0.64259 | 4995 | I.S. | 0.64256 |
| 4996 | I.S. | 0.64256 | 4997 | I.S. | 0.64255 | 4998 | I.S. | 0.64251 |
| 4999 | I.S. | 0.64244 | 5000 | I.S. | 0.64243 | 5001 | AXL | 0.64243 |
| 5002 | KMT2D | 0.64242 | 5003 | I.S. | 0.6424 | 5004 | KMT2D | 0.64239 |
| 5005 | BIRC3 | 0.64236 | 5006 | ACVR1B | 0.64236 | 5007 | GLI2 | 0.64236 |
| 5008 | I.S. | 0.64236 | 5009 | I.S. | 0.64234 | 5010 | SDHC | 0.64233 |
| 5011 | I.S. | 0.64232 | 5012 | I.S. | 0.64232 | 5013 | I.S. | 0.64232 |
| 5014 | I.S. | 0.64232 | 5015 | I.S. | 0.64231 | 5016 | I.S. | 0.64231 |
| 5017 | I.S. | 0.64229 | 5018 | I.S. | 0.64229 | 5019 | I.S. | 0.64229 |
| 5020 | KMT2D | 0.64227 | 5021 | I.S. | 0.64226 | 5022 | I.S. | 0.64226 |
| 5023 | FANCI | 0.64224 | 5024 | MITF | 0.64222 | 5025 | TSC2 | 0.64221 |
| 5026 | I.S. | 0.6422 | 5027 | LPIN2 | 0.64218 | 5028 | I.S. | 0.64218 |
| 5029 | I.S. | 0.64218 | 5030 | WT1 | 0.64218 | 5031 | ATM | 0.64215 |
| 5032 | I.S. | 0.64214 | 5033 | ATG2B | 0.64212 | 5034 | QKI | 0.64212 |
| 5035 | I.S. | 0.6421 | 5036 | TCIRG1 | 0.6421 | 5037 | I.S. | 0.64207 |
| 5038 | RAC1 | 0.64206 | 5039 | CDAN1 | 0.64206 | 5040 | I.S. | 0.64205 |
| 5041 | FGFR1 | 0.642 | 5042 | I.S. | 0.64199 | 5043 | IRF4 | 0.64197 |
| 5044 | SLX4 | 0.64195 | 5045 | I.S. | 0.64193 | 5046 | — | 0.64192 |
| 5047 | I.S. | 0.64191 | 5048 | STAT6 | 0.64187 | 5049 | I.S. | 0.64186 |
| 5050 | I.S. | 0.64185 | 5051 | I.S. | 0.64185 | 5052 | I.S. | 0.64185 |
| 5053 | I.S. | 0.64185 | 5054 | I.S. | 0.64185 | 5055 | I.S. | 0.64185 |
| 5056 | I.S. | 0.64181 | 5057 | I.S. | 0.6418 | 5058 | LRP1B | 0.64179 |
| 5059 | I.S. | 0.64179 | 5060 | I.S. | 0.64179 | 5061 | I.S. | 0.64179 |
| 5062 | FANCG | 0.64178 | 5063 | LYN | 0.64177 | 5064 | CARD11 | 0.64175 |
| 5065 | SETBP1 | 0.64174 | 5066 | I.S. | 0.64174 | 5067 | BLM | 0.64174 |
| 5068 | TCIRG1 | 0.64172 | 5069 | I.S. | 0.64166 | 5070 | ERBB3 | 0.64165 |
| 5071 | FANCA | 0.64163 | 5072 | BRIP1 | 0.64162 | 5073 | RTEL1 | 0.64162 |
| 5074 | RAD50 | 0.64162 | 5075 | I.S. | 0.64161 | 5076 | FBXW7 | 0.64159 |
| 5077 | I.S. | 0.64155 | 5078 | I.S. | 0.64155 | 5079 | I.S. | 0.64155 |
| 5080 | I.S. | 0.64155 | 5081 | CCND1 | 0.64153 | 5082 | GSK3B | 0.64153 |
| 5083 | I.S. | 0.64153 | 5084 | I.S. | 0.64149 | 5085 | I.S. | 0.64149 |
| 5086 | I.S. | 0.64149 | 5087 | NUP93 | 0.64145 | 5088 | I.S. | 0.64144 |
| 5089 | I.S. | 0.64143 | 5090 | I.S. | 0.64141 | 5091 | I.S. | 0.64139 |
| 5092 | I.S. | 0.64134 | 5093 | I.S. | 0.64134 | 5094 | KDR | 0.64133 |
| 5095 | I.S. | 0.64132 | 5096 | I.S. | 0.6413 | 5097 | I.S. | 0.6413 |
| 5098 | I.S. | 0.64128 | 5099 | I.S. | 0.64128 | 5100 | I.S. | 0.64125 |
| 5101 | I.S. | 0.64124 | 5102 | PBRM1 | 0.64123 | 5103 | TSC2 | 0.64121 |
| 5104 | I.S. | 0.6412 | 5105 | I.S. | 0.64117 | 5106 | I.S. | 0.64117 |
| 5107 | I.S. | 0.64116 | 5108 | I.S. | 0.64114 | 5109 | I.S. | 0.64111 |
| 5110 | I.S. | 0.64108 | 5111 | I.S. | 0.64107 | 5112 | QKI | 0.64103 |
| 5113 | ZBTB2 | 0.641 | 5114 | KMT2C | 0.64096 | 5115 | I.S. | 0.64096 |
| 5116 | I.S. | 0.64095 | 5117 | I.S. | 0.64095 | 5118 | DDX11 | 0.64088 |
| 5119 | EPAS1 | 0.64087 | 5120 | I.S. | 0.64086 | 5121 | CTCF | 0.64086 |
| 5122 | AKT2 | 0.64084 | 5123 | I.S. | 0.64084 | 5124 | I.S. | 0.64075 |
| 5125 | FGFR1 | 0.64075 | 5126 | I.S. | 0.64075 | 5127 | AR | 0.64073 |
| 5128 | I.S. | 0.64066 | 5129 | DDX41 | 0.64064 | 5130 | I.S. | 0.64064 |
| 5131 | I.S. | 0.64064 | 5132 | I.S. | 0.64063 | 5133 | I.S. | 0.64063 |
| 5134 | PIK3CB | 0.64061 | 5135 | I.S. | 0.64061 | 5136 | I.S. | 0.64059 |
| 5137 | I.S. | 0.64059 | 5138 | RAF1 | 0.64058 | 5139 | I.S. | 0.64058 |
| 5140 | NOTCH2 | 0.64058 | 5141 | I.S. | 0.64057 | 5142 | RUNX1T1 | 0.64055 |
| 5143 | I.S. | 0.64051 | 5144 | I.S. | 0.64051 | 5145 | I.S. | 0.64051 |
| 5146 | I.S. | 0.64051 | 5147 | I.S. | 0.64051 | 5148 | PAX5 | 0.64049 |
| 5149 | I.S. | 0.64048 | 5150 | I.S. | 0.64046 | 5151 | I.S. | 0.64045 |
| 5152 | I.S. | 0.64043 | 5153 | I.S. | 0.64043 | 5154 | IKBKE | 0.64041 |
| 5155 | I.S. | 0.64039 | 5156 | FGFR3 | 0.64037 | 5157 | I.S. | 0.64037 |
| 5158 | I.S. | 0.64035 | 5159 | I.S. | 0.64035 | 5160 | I.S. | 0.64034 |
| 5161 | I.S. | 0.64032 | 5162 | I.S. | 0.64031 | 5163 | I.S. | 0.6403 |
| 5164 | I.S. | 0.64027 | 5165 | I.S. | 0.64027 | 5166 | I.S. | 0.64023 |
| 5167 | KMT2D | 0.64022 | 5168 | I.S. | 0.64018 | 5169 | I.S. | 0.64018 |
| 5170 | I.S. | 0.64015 | 5171 | I.S. | 0.64015 | 5172 | I.S. | 0.64015 |
| 5173 | MUTYH | 0.64015 | 5174 | CCN6 | 0.6401 | 5175 | I.S. | 0.6401 |
| 5176 | MLH1 | 0.64004 | 5177 | I.S. | 0.64004 | 5178 | I.S. | 0.64004 |
| 5179 | I.S. | 0.64003 | 5180 | FLT3 | 0.64001 | 5181 | ATG2B | 0.63998 |
| 5182 | I.S. | 0.63998 | 5183 | ERBB4 | 0.63996 | 5184 | AKT2 | 0.63996 |
| 5185 | I.S. | 0.63996 | 5186 | I.S. | 0.63994 | 5187 | I.S. | 0.63994 |
| 5188 | I.S. | 0.63994 | 5189 | I.S. | 0.63993 | 5190 | GLI2 | 0.63993 |
| 5191 | ERCC4 | 0.6399 | 5192 | I.S. | 0.63987 | 5193 | ATG2B | 0.63984 |
| 5194 | PREX2 | 0.63984 | 5195 | I.S. | 0.63983 | 5196 | I.S. | 0.63982 |
| 5197 | I.S. | 0.63982 | 5198 | I.S. | 0.63982 | 5199 | CIC | 0.63981 |
| 5200 | I.S. | 0.63979 | 5201 | I.S. | 0.63978 | 5202 | FANCI | 0.63977 |
| 5203 | I.S. | 0.63977 | 5204 | I.S. | 0.63976 | 5205 | RPTOR | 0.63975 |
| 5206 | BCR | 0.63972 | 5207 | I.S. | 0.63972 | 5208 | I.S. | 0.63972 |
| 5209 | I.S. | 0.63967 | 5210 | I.S. | 0.63963 | 5211 | ABL2 | 0.63963 |
| 5212 | I.S. | 0.63961 | 5213 | ZRSR2 | 0.63961 | 5214 | LRP1B | 0.6396 |
| 5215 | VEGFA | 0.63959 | 5216 | I.S. | 0.63958 | 5217 | I.S. | 0.63957 |
| 5218 | MTOR | 0.63955 | 5219 | NF1 | 0.63954 | 5220 | I.S. | 0.63953 |
| 5221 | I.S. | 0.63947 | 5222 | I.S. | 0.63947 | 5223 | I.S. | 0.63947 |
| 5224 | I.S. | 0.63944 | 5225 | I.S. | 0.63941 | 5226 | FGFR3 | 0.6394 |
| 5227 | SF3B1 | 0.6394 | 5228 | I.S. | 0.63939 | 5229 | I.S. | 0.63937 |
| 5230 | I.S. | 0.63936 | 5231 | I.S. | 0.63936 | 5232 | FGFR1 | 0.63936 |
| 5233 | I.S. | 0.63936 | 5234 | I.S. | 0.63936 | 5235 | CHEK2 | 0.63932 |
| 5236 | I.S. | 0.63931 | 5237 | I.S. | 0.63931 | 5238 | EZH2 | 0.63927 |
| 5239 | I.S. | 0.63924 | 5240 | I.S. | 0.63924 | 5241 | I.S. | 0.63922 |
| 5242 | I.S. | 0.6392 | 5243 | TGFBR2 | 0.63918 | 5244 | I.S. | 0.63917 |
| 5245 | CREBBP | 0.63916 | 5246 | I.S. | 0.63915 | 5247 | I.S. | 0.63915 |
| 5248 | I.S. | 0.63915 | 5249 | I.S. | 0.63915 | 5250 | PDGFRB | 0.63912 |
| 5251 | I.S. | 0.63911 | 5252 | PRDM1 | 0.6391 | 5253 | I.S. | 0.6391 |
| 5254 | I.S. | 0.63906 | 5255 | I.S. | 0.63904 | 5256 | EGFR | 0.63903 |
| 5257 | XPO1 | 0.639 | 5258 | I.S. | 0.63896 | 5259 | I.S. | 0.63896 |
| 5260 | I.S. | 0.63894 | 5261 | I.S. | 0.63891 | 5262 | INPP4B | 0.63891 |
| 5263 | I.S. | 0.63889 | 5264 | I.S. | 0.63886 | 5265 | I.S. | 0.63884 |
| 5266 | I.S. | 0.63884 | 5267 | I.S. | 0.63884 | 5268 | SEC23B | 0.63883 |
| 5269 | BCL6 | 0.63882 | 5270 | I.S. | 0.63881 | 5271 | ERBB2, | 0.63879 |
| | | | | | | | MIR4728 | |
| 5272 | I.S. | 0.63878 | 5273 | I.S. | 0.63878 | 5274 | I.S. | 0.63876 |
| 5275 | I.S. | 0.63876 | 5276 | RAF1 | 0.63874 | 5277 | CHD2 | 0.63871 |
| 5278 | I.S. | 0.63869 | 5279 | LYST | 0.63868 | 5280 | PRKDC | 0.63868 |
| 5281 | I.S. | 0.63865 | 5282 | PIK3CA | 0.63862 | 5283 | I.S. | 0.63858 |
| 5284 | I.S. | 0.63854 | 5285 | NSD2 | 0.63853 | 5286 | SUFU | 0.63853 |
| 5287 | I.S. | 0.63853 | 5288 | I.S. | 0.63846 | 5289 | I.S. | 0.63843 |
| 5290 | I.S. | 0.63843 | 5291 | I.S. | 0.63841 | 5292 | I.S. | 0.6384 |
| 5293 | DNMT3A | 0.63836 | 5294 | I.S. | 0.63833 | 5295 | I.S. | 0.63832 |
| 5296 | I.S. | 0.63832 | 5297 | AK2 | 0.63827 | 5298 | I.S. | 0.63827 |
| 5299 | I.S. | 0.63821 | 5300 | I.S. | 0.63816 | 5301 | I.S. | 0.63813 |
| 5302 | I.S. | 0.63813 | 5303 | I.S. | 0.63813 | 5304 | I.S. | 0.63811 |
| 5305 | I.S. | 0.63807 | 5306 | I.S. | 0.63807 | 5307 | I.S. | 0.63807 |
| 5308 | HGF | 0.63805 | 5309 | I.S. | 0.63805 | 5310 | I.S. | 0.63802 |
| 5311 | ABL1 | 0.638 | 5312 | I.S. | 0.63797 | 5313 | AXIN2 | 0.63793 |
| 5314 | I.S. | 0.63791 | 5315 | LRP1B | 0.6379 | 5316 | I.S. | 0.63788 |
| 5317 | I.S. | 0.63788 | 5318 | I.S. | 0.63788 | 5319 | I.S. | 0.63787 |
| 5320 | I.S. | 0.63786 | 5321 | I.S. | 0.63786 | 5322 | NF2 | 0.63785 |
| 5323 | I.S. | 0.6378 | 5324 | I.S. | 0.6378 | 5325 | I.S. | 0.63778 |
| 5326 | I.S. | 0.63775 | 5327 | PIK3C2B | 0.6377 | 5328 | RAD50 | 0.6377 |
| 5329 | BRCA2 | 0.6377 | 5330 | PIK3CB | 0.63769 | 5331 | HNF1A | 0.63767 |
| 5332 | I.S. | 0.63766 | 5333 | I.S. | 0.63765 | 5334 | PBRM1 | 0.63764 |
| 5335 | I.S. | 0.63763 | 5336 | SMC3 | 0.6376 | 5337 | I.S. | 0.63758 |
| 5338 | I.S. | 0.63756 | 5339 | I.S. | 0.63756 | 5340 | I.S. | 0.63756 |
| 5341 | FGFR1 | 0.63755 | 5342 | KLF1 | 0.63755 | 5343 | LPIN2 | 0.63746 |
| 5344 | I.S. | 0.63745 | 5345 | I.S. | 0.63745 | 5346 | I.S. | 0.63744 |
| 5347 | I.S. | 0.6374 | 5348 | I.S. | 0.63739 | 5349 | SF3B1 | 0.63738 |
| 5350 | IDH2 | 0.63738 | 5351 | FGFR4 | 0.63737 | 5352 | GNAS | 0.63736 |
| 5353 | MTOR | 0.63734 | 5354 | I.S. | 0.63734 | 5355 | I.S. | 0.63734 |
| 5356 | I.S. | 0.63734 | 5357 | I.S. | 0.63734 | 5358 | I.S. | 0.63734 |
| 5359 | I.S. | 0.63734 | 5360 | I.S. | 0.63734 | 5361 | I.S. | 0.63734 |
| 5362 | I.S. | 0.63734 | 5363 | I.S. | 0.63734 | 5364 | I.S. | 0.63734 |
| 5365 | I.S. | 0.6373 | 5366 | I.S. | 0.63725 | 5367 | I.S. | 0.63725 |
| 5368 | GRIN2A | 0.63725 | 5369 | I.S. | 0.63723 | 5370 | NSD2 | 0.63722 |
| 5371 | I.S. | 0.63722 | 5372 | SETD2 | 0.6372 | 5373 | ATM | 0.6372 |
| 5374 | TSC2 | 0.63717 | 5375 | I.S. | 0.63717 | 5376 | — | 0.63716 |
| 5377 | I.S. | 0.63711 | 5378 | I.S. | 0.6371 | 5379 | I.S. | 0.63709 |
| 5380 | I.S. | 0.63709 | 5381 | I.S. | 0.63709 | 5382 | I.S. | 0.63708 |
| 5383 | FAT1 | 0.63704 | 5384 | I.S. | 0.63703 | 5385 | RPTOR | 0.63702 |
| 5386 | ARFRP1 | 0.63701 | 5387 | JAK1 | 0.637 | 5388 | I.S. | 0.63699 |
| 5389 | I.S. | 0.63698 | 5390 | I.S. | 0.63697 | 5391 | I.S. | 0.63697 |
| 5392 | NF1 | 0.63696 | 5393 | U2AF1, | 0.63693 | 5394 | I.S. | 0.63689 |
| | | | | U2AF1L5 | | | | |
| 5395 | CHEK2 | 0.63689 | 5396 | BCL6 | 0.63688 | 5397 | I.S. | 0.63688 |
| 5398 | H3F3A, | 0.63687 | 5399 | TSC1 | 0.6368 | 5400 | TERF2 | 0.63674 |
| | H3F3AP4 | | | | | | | |
| 5401 | I.S. | 0.63674 | 5402 | I.S. | 0.63673 | 5403 | KMT2D | 0.63672 |
| 5404 | KDM5A | 0.63671 | 5405 | I.S. | 0.6367 | 5406 | KDM5A | 0.63668 |
| 5407 | I.S. | 0.63667 | 5408 | I.S. | 0.63665 | 5409 | ADGRA2 | 0.63665 |
| 5410 | I.S. | 0.63665 | 5411 | RAB27A | 0.63663 | 5412 | I.S. | 0.63659 |
| 5413 | SLIT2 | 0.63657 | 5414 | TOP2A | 0.63652 | 5415 | — | 0.63652 |
| 5416 | GSKIP | 0.63651 | 5417 | I.S. | 0.63649 | 5418 | I.S. | 0.63644 |
| 5419 | I.S. | 0.63644 | 5420 | I.S. | 0.63644 | 5421 | SUFU | 0.63637 |
| 5422 | I.S. | 0.63636 | 5423 | I.S. | 0.63635 | 5424 | I.S. | 0.6363 |
| 5425 | I.S. | 0.6363 | 5426 | I.S. | 0.6363 | 5427 | I.S. | 0.63628 |
| 5428 | I.S. | 0.63626 | 5429 | I.S. | 0.63624 | 5430 | NUP93 | 0.63622 |
| 5431 | CHD4 | 0.6362 | 5432 | FBXW7 | 0.63618 | 5433 | I.S. | 0.63618 |
| 5434 | I.S. | 0.63615 | 5435 | I.S. | 0.63607 | 5436 | I.S. | 0.63607 |
| 5437 | I.S. | 0.63599 | 5438 | I.S. | 0.63599 | 5439 | I.S. | 0.63599 |
| 5440 | I.S. | 0.63599 | 5441 | I.S. | 0.63599 | 5442 | FANCD2 | 0.63598 |
| 5443 | IRF4 | 0.63598 | 5444 | TSC2 | 0.63598 | 5445 | I.S. | 0.63597 |
| 5446 | I.S. | 0.63597 | 5447 | FANCI | 0.63595 | 5448 | I.S. | 0.63594 |
| 5449 | I.S. | 0.63594 | 5450 | I.S. | 0.63594 | 5451 | I.S. | 0.63593 |
| 5452 | I.S. | 0.63593 | 5453 | CSF3R | 0.63592 | 5454 | I.S. | 0.6359 |
| 5455 | I.S. | 0.63589 | 5456 | I.S. | 0.63585 | 5457 | I.S. | 0.63584 |
| 5458 | I.S. | 0.63583 | 5459 | KIT | 0.63582 | 5460 | I.S. | 0.63582 |
| 5461 | STK11 | 0.6358 | 5462 | KMT2D | 0.63577 | 5463 | CUX1 | 0.63576 |
| 5464 | I.S. | 0.63575 | 5465 | I.S. | 0.63575 | 5466 | I.S. | 0.63573 |
| 5467 | I.S. | 0.63573 | 5468 | I.S. | 0.63569 | 5469 | PDGFRB | 0.63569 |
| 5470 | NTRK1 | 0.63568 | 5471 | I.S. | 0.63567 | 5472 | PIK3C2B | 0.63565 |
| 5473 | I.S. | 0.63565 | 5474 | I.S. | 0.63563 | 5475 | I.S. | 0.63562 |
| 5476 | I.S. | 0.63561 | 5477 | I.S. | 0.63558 | 5478 | CTNNB1 | 0.63556 |
| 5479 | I.S. | 0.63556 | 5480 | I.S. | 0.63556 | 5481 | I.S. | 0.63555 |
| 5482 | MPL | 0.63553 | 5483 | RAD50 | 0.63553 | 5484 | I.S. | 0.63553 |
| 5485 | I.S. | 0.63553 | 5486 | I.S. | 0.63549 | 5487 | DDX11 | 0.63544 |
| 5488 | I.S. | 0.63542 | 5489 | I.S. | 0.63542 | 5490 | I.S. | 0.63542 |
| 5491 | FLT4 | 0.63541 | 5492 | ARID1A | 0.63541 | 5493 | FLT3 | 0.63541 |
| 5494 | I.S. | 0.63537 | 5495 | I.S. | 0.63535 | 5496 | I.S. | 0.63535 |
| 5497 | I.S. | 0.63532 | 5498 | BCL2L1 | 0.63529 | 5499 | I.S. | 0.63528 |
| 5500 | I.S. | 0.63528 | 5501 | I.S. | 0.63528 | 5502 | CBL | 0.63527 |
| 5503 | I.S. | 0.63523 | 5504 | GLI1 | 0.63523 | 5505 | MITF | 0.63522 |
| 5506 | MET | 0.63521 | 5507 | I.S. | 0.6352 | 5508 | I.S. | 0.63519 |
| 5509 | ERBB4 | 0.63515 | 5510 | MAGI2 | 0.63508 | 5511 | CTCF | 0.63508 |
| 5512 | I.S. | 0.63508 | 5513 | I.S. | 0.63507 | 5514 | I.S. | 0.63507 |
| 5515 | I.S. | 0.63506 | 5516 | ABL2 | 0.63506 | 5517 | FLCN | 0.63505 |
| 5518 | I.S. | 0.63504 | 5519 | I.S. | 0.63504 | 5520 | PIK3C2B | 0.63502 |
| 5521 | FGFR3 | 0.635 | 5522 | I.S. | 0.63498 | 5523 | I.S. | 0.63497 |
| 5524 | LRP1B | 0.63496 | 5525 | I.S. | 0.63495 | 5526 | I.S. | 0.63495 |
| 5527 | I.S. | 0.63495 | 5528 | I.S. | 0.63495 | 5529 | I.S. | 0.63495 |
| 5530 | I.S. | 0.63495 | 5531 | I.S. | 0.63494 | 5532 | I.S. | 0.63492 |
| 5533 | ACVR1B | 0.63487 | 5534 | I.S. | 0.63486 | 5535 | I.S. | 0.63484 |
| 5536 | CDK6 | 0.63481 | 5537 | KLHL6 | 0.63476 | 5538 | TOP2A | 0.63475 |
| 5539 | SMARCA4 | 0.63474 | 5540 | RAF1 | 0.63472 | 5541 | I.S. | 0.63471 |
| 5542 | I.S. | 0.63471 | 5543 | SLIT2 | 0.63471 | 5544 | STAT3 | 0.6347 |
| 5545 | PIM1 | 0.63467 | 5546 | SRSF2, | 0.63467 | 5547 | I.S. | 0.63467 |
| | | | | MIR636 | | | | |
| 5548 | SFTA3, | 0.63464 | 5549 | AXIN1 | 0.63464 | 5550 | I.S. | 0.63463 |
| | NKX2-1 | | | | | | | |
| 5551 | I.S. | 0.63463 | 5552 | I.S. | 0.63463 | 5553 | I.S. | 0.63463 |
| 5554 | I.S. | 0.63459 | 5555 | ERBB3 | 0.63459 | 5556 | I.S. | 0.63457 |
| 5557 | I.S. | 0.63453 | 5558 | DNMT3A | 0.63449 | 5559 | I.S. | 0.63449 |
| 5560 | TSC2 | 0.63443 | 5561 | I.S. | 0.63438 | 5562 | I.S. | 0.63438 |
| 5563 | I.S. | 0.63438 | 5564 | I.S. | 0.63438 | 5565 | I.S. | 0.63435 |
| 5566 | I.S. | 0.63435 | 5567 | SUFU | 0.63431 | 5568 | I.S. | 0.63428 |
| 5569 | I.S. | 0.63428 | 5570 | I.S. | 0.63425 | 5571 | I.S. | 0.63424 |
| 5572 | I.S. | 0.63423 | 5573 | PIK3C2B | 0.63423 | 5574 | ASXL1 | 0.63423 |
| 5575 | TENT5C | 0.63416 | 5576 | I.S. | 0.63416 | 5577 | I.S. | 0.63416 |
| 5578 | DNM2, | 0.63416 | 5579 | I.S. | 0.63413 | 5580 | ATM | 0.63409 |
| | MIR638 | | | | | | | |
| 5581 | I.S. | 0.63405 | 5582 | PRKCI | 0.63404 | 5583 | I.S. | 0.63404 |
| 5584 | I.S. | 0.63404 | 5585 | I.S. | 0.63402 | 5586 | TNFAIP3 | 0.634 |
| 5587 | I.S. | 0.63399 | 5588 | I.S. | 0.63398 | 5589 | I.S. | 0.63397 |
| 5590 | KDR | 0.63396 | 5591 | I.S. | 0.63395 | 5592 | I.S. | 0.63394 |
| 5593 | I.S. | 0.63392 | 5594 | I.S. | 0.63392 | 5595 | I.S. | 0.63392 |
| 5596 | I.S. | 0.63392 | 5597 | I.S. | 0.63392 | 5598 | I.S. | 0.63392 |
| 5599 | I.S. | 0.63392 | 5600 | I.S. | 0.63392 | 5601 | I.S. | 0.63392 |
| 5602 | I.S. | 0.63392 | 5603 | I.S. | 0.63392 | 5604 | I.S. | 0.63388 |
| 5605 | ACD | 0.63388 | 5606 | LPIN2 | 0.63388 | 5607 | I.S. | 0.63386 |
| 5608 | I.S. | 0.63386 | 5609 | I.S. | 0.63386 | 5610 | NFE2L2 | 0.63384 |
| 5611 | I.S. | 0.63384 | 5612 | RAF1 | 0.63383 | 5613 | I.S. | 0.63383 |
| 5614 | BRIP1 | 0.63383 | 5615 | I.S. | 0.6338 | 5616 | I.S. | 0.6338 |
| 5617 | I.S. | 0.6338 | 5618 | I.S. | 0.63373 | 5619 | I.S. | 0.63372 |
| 5620 | MYCL | 0.63371 | 5621 | I.S. | 0.6337 | 5622 | ADGRA2 | 0.63369 |
| 5623 | I.S. | 0.63368 | 5624 | I.S. | 0.63368 | 5625 | I.S. | 0.63366 |
| 5626 | I.S. | 0.63363 | 5627 | KEL | 0.6336 | 5628 | PDGFRB | 0.6336 |
| 5629 | I.S. | 0.63359 | 5630 | I.S. | 0.63359 | 5631 | I.S. | 0.63358 |
| 5632 | RTEL1 | 0.63358 | 5633 | I.S. | 0.63356 | 5634 | I.S. | 0.63356 |
| 5635 | EPHA7 | 0.6335 | 5636 | I.S. | 0.63349 | 5637 | I.S. | 0.63347 |
| 5638 | I.S. | 0.63346 | 5639 | CHD4 | 0.63342 | 5640 | LYN | 0.63336 |
| 5641 | I.S. | 0.63334 | 5642 | I.S. | 0.63328 | 5643 | FANCM | 0.63328 |
| 5644 | I.S. | 0.63328 | 5645 | I.S. | 0.63326 | 5646 | I.S. | 0.63326 |
| 5647 | I.S. | 0.63325 | 5648 | I.S. | 0.63325 | 5649 | I.S. | 0.63323 |
| 5650 | I.S. | 0.63321 | 5651 | I.S. | 0.63319 | 5652 | JAGN1 | 0.63319 |
| 5653 | I.S. | 0.63319 | 5654 | I.S. | 0.63318 | 5655 | — | 0.63318 |
| 5656 | I.S. | 0.63318 | 5657 | I.S. | 0.63318 | 5658 | I.S. | 0.63314 |
| 5659 | IKBKE | 0.63313 | 5660 | I.S. | 0.6331 | 5661 | I.S. | 0.63309 |
| 5662 | PIK3CG | 0.63306 | 5663 | I.S. | 0.63306 | 5664 | KDM5A | 0.63306 |
| 5665 | PBRM1 | 0.63302 | 5666 | I.S. | 0.63299 | 5667 | TERT | 0.63298 |
| 5668 | MTOR | 0.63298 | 5669 | TSC1 | 0.63298 | 5670 | BCL6 | 0.63297 |
| 5671 | FANCI | 0.63295 | 5672 | CDK4 | 0.63294 | 5673 | I.S. | 0.63293 |
| 5674 | I.S. | 0.63292 | 5675 | I.S. | 0.6329 | 5676 | I.S. | 0.6329 |
| 5677 | IKZF1 | 0.63289 | 5678 | I.S. | 0.63288 | 5679 | I.S. | 0.63288 |
| 5680 | I.S. | 0.63288 | 5681 | I.S. | 0.63288 | 5682 | I.S. | 0.63287 |
| 5683 | KMT2B | 0.63286 | 5684 | I.S. | 0.63281 | 5685 | I.S. | 0.6328 |
| 5686 | SPTA1 | 0.6328 | 5687 | EPHA7 | 0.63279 | 5688 | GEN1 | 0.63277 |
| 5689 | I.S. | 0.63276 | 5690 | I.S. | 0.63276 | 5691 | PTCH1 | 0.63274 |
| 5692 | AURKC | 0.63274 | 5693 | RHOA | 0.63273 | 5694 | I.S. | 0.63273 |
| 5695 | HAX1 | 0.63272 | 5696 | IDH2 | 0.6327 | 5697 | AKT1 | 0.63269 |
| 5698 | I.S. | 0.63269 | 5699 | I.S. | 0.63269 | 5700 | MLH1 | 0.63268 |
| 5701 | I.S. | 0.63266 | 5702 | SUZ12 | 0.63265 | 5703 | POLE | 0.63265 |
| 5704 | I.S. | 0.63263 | 5705 | RAD51D | 0.63262 | 5706 | ROS1 | 0.63261 |
| 5707 | BMPR1A | 0.63261 | 5708 | TERF2 | 0.63259 | 5709 | I.S. | 0.63257 |
| 5710 | I.S. | 0.63256 | 5711 | ATG2B | 0.63256 | 5712 | PDGFRA | 0.63256 |
| 5713 | I.S. | 0.63256 | 5714 | AURKA | 0.63253 | 5715 | I.S. | 0.63252 |
| 5716 | SMAD3 | 0.6325 | 5717 | I.S. | 0.63246 | 5718 | EZH2 | 0.63245 |
| 5719 | I.S. | 0.63244 | 5720 | I.S. | 0.63243 | 5721 | I.S. | 0.63241 |
| 5722 | I.S. | 0.6324 | 5723 | SNCAIP | 0.63238 | 5724 | I.S. | 0.63236 |
| 5725 | MTOR | 0.63229 | 5726 | RAD50 | 0.63228 | 5727 | I.S. | 0.63227 |
| 5728 | BRCA2 | 0.63226 | 5729 | I.S. | 0.6322 | 5730 | I.S. | 0.63219 |
| 5731 | NOTCH2 | 0.63215 | 5732 | ADA | 0.63215 | 5733 | CHD4 | 0.63214 |
| 5734 | ARID1B | 0.63214 | 5735 | I.S. | 0.63213 | 5736 | I.S. | 0.63213 |
| 5737 | PRDM1 | 0.63212 | 5738 | FANCD2 | 0.63211 | 5739 | I.S. | 0.63211 |
| 5740 | I.S. | 0.6321 | 5741 | HNF1A | 0.63208 | 5742 | I.S. | 0.63207 |
| 5743 | AKT3 | 0.63205 | 5744 | I.S. | 0.63205 | 5745 | I.S. | 0.63202 |
| 5746 | I.S. | 0.63199 | 5747 | I.S. | 0.63192 | 5748 | ASXL1 | 0.6319 |
| 5749 | I.S. | 0.6319 | 5750 | RPTOR | 0.63188 | 5751 | I.S. | 0.63186 |
| 5752 | ATR | 0.63185 | 5753 | RTEL1 | 0.63185 | 5754 | PMS2 | 0.63181 |
| 5755 | I.S. | 0.63178 | 5756 | I.S. | 0.63178 | 5757 | I.S. | 0.63178 |
| 5758 | I.S. | 0.63178 | 5759 | I.S. | 0.63178 | 5760 | I.S. | 0.63178 |
| 5761 | ADGRA2 | 0.63172 | 5762 | I.S. | 0.6317 | 5763 | I.S. | 0.63166 |
| 5764 | I.S. | 0.63164 | 5765 | I.S. | 0.63161 | 5766 | FLT1 | 0.63161 |
| 5767 | CSF3R | 0.63161 | 5768 | I.S. | 0.63159 | 5769 | I.S. | 0.63154 |
| 5770 | I.S. | 0.63154 | 5771 | I.S. | 0.63154 | 5772 | I.S. | 0.63152 |
| 5773 | I.S. | 0.6315 | 5774 | I.S. | 0.63145 | 5775 | I.S. | 0.63145 |
| 5776 | I.S. | 0.63145 | 5777 | NTRK3 | 0.63145 | 5778 | I.S. | 0.63142 |
| 5779 | POLE | 0.63138 | 5780 | I.S. | 0.63137 | 5781 | I.S. | 0.63136 |
| 5782 | BRCA1 | 0.63136 | 5783 | CHD4 | 0.63134 | 5784 | CARD11 | 0.63134 |
| 5785 | I.S. | 0.63131 | 5786 | I.S. | 0.63128 | 5787 | I.S. | 0.63124 |
| 5788 | I.S. | 0.63121 | 5789 | I.S. | 0.63121 | 5790 | I.S. | 0.63119 |
| 5791 | I.S. | 0.63117 | 5792 | I.S. | 0.63113 | 5793 | I.S. | 0.63113 |
| 5794 | I.S. | 0.63113 | 5795 | I.S. | 0.63113 | 5796 | I.S. | 0.6311 |
| 5797 | I.S. | 0.63108 | 5798 | TERF2 | 0.63108 | 5799 | I.S. | 0.63107 |
| 5800 | RB1 | 0.63105 | 5801 | PDGFRB | 0.63104 | 5802 | KMT2A | 0.63102 |
| 5803 | SMC3 | 0.63101 | 5804 | I.S. | 0.63099 | 5805 | SMAD4 | 0.63098 |
| 5806 | I.S. | 0.63097 | 5807 | I.S. | 0.63095 | 5808 | PIK3CG | 0.63093 |
| 5809 | BRCA1 | 0.63092 | 5810 | I.S. | 0.63088 | 5811 | I.S. | 0.63087 |
| 5812 | CALR | 0.63087 | 5813 | ATG2B | 0.63086 | 5814 | I.S. | 0.63085 |
| 5815 | I.S. | 0.63085 | 5816 | I.S. | 0.63085 | 5817 | I.S. | 0.63085 |
| 5818 | SRP19, | 0.63084 | 5819 | FANCM | 0.63084 | 5820 | CBFB | 0.63082 |
| | REEP5 | | | | | | | |
| 5821 | I.S. | 0.63082 | 5822 | I.S. | 0.63079 | 5823 | I.S. | 0.63076 |
| 5824 | I.S. | 0.63076 | 5825 | I.S. | 0.63074 | 5826 | I.S. | 0.63074 |
| 5827 | I.S. | 0.63074 | 5828 | I.S. | 0.63074 | 5829 | I.S. | 0.63074 |
| 5830 | I.S. | 0.63071 | 5831 | I.S. | 0.63068 | 5832 | ALK | 0.63064 |
| 5833 | I.S. | 0.63063 | 5834 | RAC1 | 0.63062 | 5835 | I.S. | 0.63057 |
| 5836 | CBL | 0.63057 | 5837 | TCF3 | 0.63057 | 5838 | I.S. | 0.63056 |
| 5839 | MCL1 | 0.63054 | 5840 | FLT1 | 0.63054 | 5841 | FANCD2 | 0.63045 |
| 5842 | PRKCI | 0.63041 | 5843 | BLM | 0.63039 | 5844 | I.S. | 0.63038 |
| 5845 | I.S. | 0.63038 | 5846 | I.S. | 0.63038 | 5847 | I.S. | 0.63035 |
| 5848 | I.S. | 0.63033 | 5849 | I.S. | 0.63031 | 5850 | I.S. | 0.63031 |
| 5851 | I.S. | 0.6303 | 5852 | I.S. | 0.6303 | 5853 | PLCG1 | 0.63026 |
| 5854 | NBPF20, | 0.63026 | 5855 | APC | 0.63024 | 5856 | APC | 0.63024 |
| | NBPF19, | | | | | | | |
| | NBPF10, | | | | | | | |
| | RBM8A | | | | | | | |
| 5857 | I.S. | 0.63018 | 5858 | I.S. | 0.63017 | 5859 | I.S. | 0.63017 |
| 5860 | I.S. | 0.63015 | 5861 | PTCH1 | 0.63015 | 5862 | I.S. | 0.63013 |
| 5863 | I.S. | 0.63011 | 5864 | I.S. | 0.63011 | 5865 | I.S. | 0.6301 |
| 5866 | I.S. | 0.63009 | 5867 | I.S. | 0.63008 | 5868 | I.S. | 0.63005 |
| 5869 | PSTPIP1 | 0.63003 | 5870 | I.S. | 0.62997 | 5871 | I.S. | 0.62994 |
| 5872 | I.S. | 0.62994 | 5873 | RPTOR | 0.62992 | 5874 | I.S. | 0.6299 |
| 5875 | CCN6 | 0.62988 | 5876 | I.S. | 0.62987 | 5877 | DAXX | 0.62985 |
| 5878 | BRD4 | 0.62982 | 5879 | AKT2 | 0.6298 | 5880 | ATM | 0.6298 |
| 5881 | I.S. | 0.62978 | 5882 | DDX41 | 0.62977 | 5883 | CDAN1 | 0.62976 |
| 5884 | I.S. | 0.62976 | 5885 | BRD4 | 0.62974 | 5886 | I.S. | 0.62968 |
| 5887 | I.S. | 0.62967 | 5888 | ATG2B | 0.62965 | 5889 | I.S. | 0.62964 |
| 5890 | I.S. | 0.62964 | 5891 | I.S. | 0.62964 | 5892 | I.S. | 0.62964 |
| 5893 | I.S. | 0.62961 | 5894 | I.S. | 0.62959 | 5895 | I.S. | 0.62957 |
| 5896 | I.S. | 0.62955 | 5897 | I.S. | 0.62954 | 5898 | I.S. | 0.6295 |
| 5899 | I.S. | 0.62948 | 5900 | NF1 | 0.62944 | 5901 | I.S. | 0.62944 |
| 5902 | ARID1B | 0.62943 | 5903 | I.S. | 0.6294 | 5904 | I.S. | 0.6294 |
| 5905 | I.S. | 0.6294 | 5906 | I.S. | 0.6294 | 5907 | SPOP | 0.62936 |
| 5908 | KMT2D | 0.62936 | 5909 | AK2 | 0.62935 | 5910 | PIK3CA | 0.62935 |
| 5911 | I.S. | 0.62934 | 5912 | I.S. | 0.62934 | 5913 | ATR | 0.62933 |
| 5914 | I.S. | 0.62932 | 5915 | I.S. | 0.62932 | 5916 | I.S. | 0.62932 |
| 5917 | I.S. | 0.62932 | 5918 | I.S. | 0.6293 | 5919 | KMT2D | 0.62929 |
| 5920 | I.S. | 0.62927 | 5921 | I.S. | 0.62926 | 5922 | PLCG2 | 0.6292 |
| 5923 | I.S. | 0.6292 | 5924 | I.S. | 0.62918 | 5925 | I.S. | 0.62915 |
| 5926 | I.S. | 0.62915 | 5927 | I.S. | 0.62915 | 5928 | INHBA | 0.62915 |
| 5929 | I.S. | 0.62915 | 5930 | I.S. | 0.62914 | 5931 | ERCC4 | 0.62914 |
| 5932 | IGF1R | 0.62914 | 5933 | ARID1A | 0.62911 | 5934 | ATR | 0.62911 |
| 5935 | I.S. | 0.62907 | 5936 | I.S. | 0.62907 | 5937 | I.S. | 0.62906 |
| 5938 | TOP2A | 0.62906 | 5939 | I.S. | 0.62906 | 5940 | I.S. | 0.62904 |
| 5941 | I.S. | 0.62902 | 5942 | I.S. | 0.62899 | 5943 | RNF168 | 0.62897 |
| 5944 | I.S. | 0.62895 | 5945 | AK2 | 0.62893 | 5946 | I.S. | 0.62893 |
| 5947 | I.S. | 0.62892 | 5948 | INPP4B | 0.62891 | 5949 | I.S. | 0.62886 |
| 5950 | APC | 0.62885 | 5951 | TOP2A | 0.62885 | 5952 | I.S. | 0.62885 |
| 5953 | I.S. | 0.62883 | 5954 | I.S. | 0.62879 | 5955 | I.S. | 0.62879 |
| 5956 | I.S. | 0.62878 | 5957 | I.S. | 0.62872 | 5958 | I.S. | 0.62871 |
| 5959 | I.S. | 0.62871 | 5960 | CHEK1 | 0.62871 | 5961 | I.S. | 0.6287 |
| 5962 | DOT1L | 0.6287 | 5963 | I.S. | 0.62869 | 5964 | DDR2 | 0.62868 |
| 5965 | I.S. | 0.62863 | 5966 | I.S. | 0.62863 | 5967 | I.S. | 0.6286 |
| 5968 | MTOR | 0.6286 | 5969 | I.S. | 0.62859 | 5970 | CREBBP | 0.62858 |
| 5971 | CTC1 | 0.62858 | 5972 | PDK1 | 0.62856 | 5973 | MYCNOS, | 0.62855 |
| | | | | | | | MYCN, MYCN | |
| 5974 | TET2 | 0.62855 | 5975 | I.S. | 0.62854 | 5976 | I.S. | 0.62849 |
| 5977 | PDGFRA | 0.62849 | 5978 | MDM4 | 0.62846 | 5979 | FGFR3 | 0.6284 |
| 5980 | I.S. | 0.62838 | 5981 | I.S. | 0.62836 | 5982 | I.S. | 0.62836 |
| 5983 | I.S. | 0.62836 | 5984 | I.S. | 0.62836 | 5985 | EP300 | 0.62834 |
| 5986 | I.S. | 0.62832 | 5987 | I.S. | 0.62831 | 5988 | I.S. | 0.62828 |
| 5989 | BRIP1 | 0.62828 | 5990 | I.S. | 0.62828 | 5991 | I.S. | 0.62827 |
| 5992 | I.S. | 0.62827 | 5993 | I.S. | 0.62826 | 5994 | I.S. | 0.62824 |
| 5995 | I.S. | 0.62824 | 5996 | I.S. | 0.62824 | 5997 | I.S. | 0.62824 |
| 5998 | I.S. | 0.62822 | 5999 | LRP1B | 0.6282 | 6000 | I.S. | 0.62816 |
| 6001 | I.S. | 0.62812 | 6002 | I.S. | 0.62812 | 6003 | I.S. | 0.62811 |
| 6004 | I.S. | 0.62808 | 6005 | SUFU | 0.62808 | 6006 | DDX11 | 0.62808 |
| 6007 | I.S. | 0.62806 | 6008 | FGFR1 | 0.62804 | 6009 | I.S. | 0.62803 |
| 6010 | SMC1A | 0.62802 | 6011 | I.S. | 0.628 | 6012 | I.S. | 0.628 |
| 6013 | I.S. | 0.62799 | 6014 | I.S. | 0.62799 | 6015 | STAT4 | 0.62799 |
| 6016 | CHD4 | 0.62799 | 6017 | I.S. | 0.62799 | 6018 | I.S. | 0.62797 |
| 6019 | TOP2A | 0.62796 | 6020 | SRC | 0.62796 | 6021 | I.S. | 0.62795 |
| 6022 | I.S. | 0.6279 | 6023 | I.S. | 0.62789 | 6024 | I.S. | 0.62787 |
| 6025 | I.S. | 0.62783 | 6026 | I.S. | 0.62783 | 6027 | I.S. | 0.62781 |
| 6028 | I.S. | 0.62779 | 6029 | CBLB | 0.62778 | 6030 | DDR2 | 0.62778 |
| 6031 | I.S. | 0.62776 | 6032 | I.S. | 0.62774 | 6033 | STAT3 | 0.62774 |
| 6034 | I.S. | 0.62773 | 6035 | SMAD3 | 0.62772 | 6036 | FAT1 | 0.62772 |
| 6037 | I.S. | 0.62772 | 6038 | I.S. | 0.62771 | 6039 | I.S. | 0.62771 |
| 6040 | SAMD9L | 0.62769 | 6041 | I.S. | 0.62767 | 6042 | I.S. | 0.62765 |
| 6043 | I.S. | 0.62765 | 6044 | ARID1A | 0.62764 | 6045 | I.S. | 0.62763 |
| 6046 | I.S. | 0.62761 | 6047 | I.S. | 0.6276 | 6048 | MRE11 | 0.62759 |
| 6049 | I.S. | 0.62757 | 6050 | I.S. | 0.62757 | 6051 | I.S. | 0.62757 |
| 6052 | I.S. | 0.62747 | 6053 | I.S. | 0.62747 | 6054 | I.S. | 0.62745 |
| 6055 | CHD4 | 0.62745 | 6056 | I.S. | 0.62745 | 6057 | GSK3B | 0.62739 |
| 6058 | I.S. | 0.62738 | 6059 | TSC1 | 0.62734 | 6060 | I.S. | 0.62732 |
| 6061 | I.S. | 0.62732 | 6062 | TSC2 | 0.62731 | 6063 | I.S. | 0.62729 |
| 6064 | LYST | 0.62727 | 6065 | I.S. | 0.62727 | 6066 | I.S. | 0.62726 |
| 6067 | I.S. | 0.62726 | 6068 | I.S. | 0.62724 | 6069 | NOTCH3 | 0.62721 |
| 6070 | I.S. | 0.62719 | 6071 | I.S. | 0.62717 | 6072 | ERBB2 | 0.62709 |
| 6073 | ERBB3 | 0.62709 | 6074 | CDH1 | 0.62706 | 6075 | I.S. | 0.62704 |
| 6076 | I.S. | 0.62702 | 6077 | I.S. | 0.62702 | 6078 | I.S. | 0.62702 |
| 6079 | ARID2 | 0.62701 | 6080 | I.S. | 0.62694 | 6081 | FANCD2 | 0.62694 |
| 6082 | I.S. | 0.62689 | 6083 | I.S. | 0.62688 | 6084 | I.S. | 0.62688 |
| 6085 | I.S. | 0.62685 | 6086 | I.S. | 0.62679 | 6087 | SMAD9 | 0.62679 |
| 6088 | EGLN1 | 0.62668 | 6089 | KDM5A | 0.62665 | 6090 | RARA | 0.62665 |
| 6091 | I.S. | 0.62664 | 6092 | I.S. | 0.62664 | 6093 | MYCL | 0.62659 |
| 6094 | NOTCH3 | 0.62655 | 6095 | ABCB7 | 0.62653 | 6096 | KAT6A | 0.62653 |
| 6097 | I.S. | 0.6265 | 6098 | I.S. | 0.62647 | 6099 | I.S. | 0.62647 |
| 6100 | I.S. | 0.62645 | 6101 | I.S. | 0.62643 | 6102 | KMT2B | 0.62641 |
| 6103 | ANKRD26 | 0.62639 | 6104 | I.S. | 0.62636 | 6105 | I.S. | 0.62636 |
| 6106 | AXIN1 | 0.62632 | 6107 | I.S. | 0.62629 | 6108 | FOXP1 | 0.62628 |
| 6109 | FANCL | 0.62627 | 6110 | FANCB | 0.62627 | 6111 | I.S. | 0.62626 |
| 6112 | I.S. | 0.62622 | 6113 | I.S. | 0.62622 | 6114 | I.S. | 0.62622 |
| 6115 | I.S. | 0.62622 | 6116 | I.S. | 0.62622 | 6117 | I.S. | 0.62622 |
| 6118 | I.S. | 0.62622 | 6119 | I.S. | 0.62614 | 6120 | I.S. | 0.62614 |
| 6121 | SNCAIP | 0.62612 | 6122 | I.S. | 0.6261 | 6123 | NROB1 | 0.62607 |
| 6124 | I.S. | 0.62606 | 6125 | PIK3CG | 0.62606 | 6126 | I.S. | 0.62602 |
| 6127 | I.S. | 0.626 | 6128 | I.S. | 0.62598 | 6129 | I.S. | 0.62598 |
| 6130 | I.S. | 0.62598 | 6131 | I.S. | 0.62598 | 6132 | I.S. | 0.62598 |
| 6133 | I.S. | 0.62598 | 6134 | I.S. | 0.62597 | 6135 | I.S. | 0.62591 |
| 6136 | I.S. | 0.6259 | 6137 | I.S. | 0.6259 | 6138 | I.S. | 0.6259 |
| 6139 | MSH6 | 0.62586 | 6140 | ZBTB2 | 0.62575 | 6141 | AKT3 | 0.62575 |
| 6142 | TET2 | 0.62575 | 6143 | SYK | 0.62573 | 6144 | JAK1 | 0.62572 |
| 6145 | RAC1 | 0.62572 | 6146 | I.S. | 0.62568 | 6147 | ADA | 0.62567 |
| 6148 | I.S. | 0.62565 | 6149 | I.S. | 0.62564 | 6150 | I.S. | 0.62562 |
| 6151 | I.S. | 0.62562 | 6152 | I.S. | 0.62561 | 6153 | SMC3 | 0.62555 |
| 6154 | I.S. | 0.62555 | 6155 | I.S. | 0.62555 | 6156 | RIT1 | 0.62551 |
| 6157 | I.S. | 0.62551 | 6158 | I.S. | 0.6255 | 6159 | DNMT3A | 0.62549 |
| 6160 | AXIN2 | 0.62549 | 6161 | I.S. | 0.62549 | 6162 | I.S. | 0.62546 |
| 6163 | VEGFA | 0.62546 | 6164 | I.S. | 0.62543 | 6165 | I.S. | 0.62543 |
| 6166 | I.S. | 0.62543 | 6167 | DNM2 | 0.62541 | 6168 | TNFAIP3 | 0.6254 |
| 6169 | CTC1 | 0.62537 | 6170 | I.S. | 0.62535 | 6171 | I.S. | 0.62533 |
| 6172 | FANCD2 | 0.62532 | 6173 | I.S. | 0.62529 | 6174 | ACD | 0.62528 |
| 6175 | I.S. | 0.62525 | 6176 | I.S. | 0.62524 | 6177 | BLM | 0.6252 |
| 6178 | I.S. | 0.62519 | 6179 | I.S. | 0.62519 | 6180 | SMAD4 | 0.62515 |
| 6181 | I.S. | 0.62514 | 6182 | FANCM | 0.62513 | 6183 | I.S. | 0.62513 |
| 6184 | I.S. | 0.62511 | 6185 | I.S. | 0.6251 | 6186 | I.S. | 0.6251 |
| 6187 | I.S. | 0.62508 | 6188 | I.S. | 0.62508 | 6189 | I.S. | 0.62507 |
| 6190 | I.S. | 0.62507 | 6191 | ATR | 0.62507 | 6192 | IKBKE | 0.62505 |
| 6193 | I.S. | 0.62501 | 6194 | AXIN1 | 0.62499 | 6195 | CARD11 | 0.62496 |
| 6196 | I.S. | 0.62494 | 6197 | TSC2 | 0.62493 | 6198 | I.S. | 0.62488 |
| 6199 | I.S. | 0.62488 | 6200 | I.S. | 0.62488 | 6201 | I.S. | 0.62488 |
| 6202 | NUP93 | 0.62487 | 6203 | I.S. | 0.62486 | 6204 | LRP1B | 0.62484 |
| 6205 | BCL2 | 0.62484 | 6206 | I.S. | 0.62484 | 6207 | I.S. | 0.62483 |
| 6208 | I.S. | 0.62483 | 6209 | SPTA1 | 0.6248 | 6210 | I.S. | 0.6248 |
| 6211 | AKT3 | 0.62477 | 6212 | I.S. | 0.62476 | 6213 | I.S. | 0.62475 |
| 6214 | SBF2 | 0.62472 | 6215 | CDAN1 | 0.62472 | 6216 | TCIRG1 | 0.62469 |
| 6217 | I.S. | 0.62458 | 6218 | I.S. | 0.62455 | 6219 | I.S. | 0.62454 |
| 6220 | I.S. | 0.62453 | 6221 | NOTCH3 | 0.62451 | 6222 | I.S. | 0.6245 |
| 6223 | I.S. | 0.6245 | 6224 | EZH2 | 0.62443 | 6225 | NSD2 | 0.62442 |
| 6226 | I.S. | 0.62441 | 6227 | I.S. | 0.62437 | 6228 | MEFV | 0.62437 |
| 6229 | KMT2C | 0.62436 | 6230 | I.S. | 0.62433 | 6231 | STAT6 | 0.6243 |
| 6232 | ERBB3 | 0.6243 | 6233 | I.S. | 0.62429 | 6234 | SBF2 | 0.62425 |
| 6235 | RTEL1 | 0.62424 | 6236 | I.S. | 0.62419 | 6237 | I.S. | 0.62417 |
| 6238 | RPTOR | 0.6241 | 6239 | BRCA2 | 0.62409 | 6240 | NLRP3 | 0.62409 |
| 6241 | I.S. | 0.62409 | 6242 | MLH1 | 0.62404 | 6243 | I.S. | 0.62404 |
| 6244 | I.S. | 0.624 | 6245 | FLT3 | 0.624 | 6246 | TAL1 | 0.624 |
| 6247 | I.S. | 0.624 | 6248 | I.S. | 0.62397 | 6249 | PRDM1 | 0.62388 |
| 6250 | I.S. | 0.62384 | 6251 | I.S. | 0.62384 | 6252 | I.S. | 0.62384 |
| 6253 | I.S. | 0.62384 | 6254 | CEBPA | 0.62383 | 6255 | GLI2 | 0.62383 |
| 6256 | I.S. | 0.62382 | 6257 | I.S. | 0.62381 | 6258 | LYN | 0.6238 |
| 6259 | BCR | 0.6238 | 6260 | I.S. | 0.62379 | 6261 | I.S. | 0.62376 |
| 6262 | I.S. | 0.62376 | 6263 | I.S. | 0.62376 | 6264 | BARD1 | 0.62374 |
| 6265 | I.S. | 0.62374 | 6266 | I.S. | 0.62373 | 6267 | I.S. | 0.62372 |
| 6268 | MAP3K14 | 0.62371 | 6269 | I.S. | 0.62371 | 6270 | I.S. | 0.62364 |
| 6271 | CHD4 | 0.62359 | 6272 | TCF3 | 0.62359 | 6273 | I.S. | 0.62354 |
| 6274 | I.S. | 0.62352 | 6275 | I.S. | 0.62347 | 6276 | NFE2L2 | 0.62347 |
| 6277 | DOT1L | 0.62347 | 6278 | NSD1 | 0.62347 | 6279 | I.S. | 0.62347 |
| 6280 | I.S. | 0.62345 | 6281 | I.S. | 0.62344 | 6282 | I.S. | 0.62343 |
| 6283 | I.S. | 0.62342 | 6284 | I.S. | 0.6234 | 6285 | POLE | 0.62338 |
| 6286 | I.S. | 0.62338 | 6287 | I.S. | 0.62338 | 6288 | CHD4 | 0.62334 |
| 6289 | TCIRG1 | 0.62332 | 6290 | I.S. | 0.62329 | 6291 | PIM1 | 0.62327 |
| 6292 | MTOR | 0.62319 | 6293 | I.S. | 0.62318 | 6294 | I.S. | 0.62316 |
| 6295 | I.S. | 0.62315 | 6296 | I.S. | 0.62313 | 6297 | I.S. | 0.6231 |
| 6298 | I.S. | 0.62307 | 6299 | I.S. | 0.62306 | 6300 | I.S. | 0.62305 |
| 6301 | I.S. | 0.62305 | 6302 | I.S. | 0.62305 | 6303 | I.S. | 0.62304 |
| 6304 | TOP1 | 0.62303 | 6305 | I.S. | 0.62302 | 6306 | TOP2A | 0.62301 |
| 6307 | I.S. | 0.623 | 6308 | I.S. | 0.62299 | 6309 | I.S. | 0.62297 |
| 6310 | I.S. | 0.62296 | 6311 | I.S. | 0.62294 | 6312 | I.S. | 0.62292 |
| 6313 | ESR1 | 0.62291 | 6314 | RPTOR | 0.62291 | 6315 | SMARCA4 | 0.62288 |
| 6316 | I.S. | 0.62288 | 6317 | SNCAIP | 0.62285 | 6318 | RNF43 | 0.62285 |
| 6319 | PREX2 | 0.62284 | 6320 | NOTCH2 | 0.62282 | 6321 | I.S. | 0.62282 |
| 6322 | POT1 | 0.62282 | 6323 | I.S. | 0.62281 | 6324 | I.S. | 0.62281 |
| 6325 | SNCAIP | 0.62278 | 6326 | ALK | 0.62269 | 6327 | I.S. | 0.62261 |
| 6328 | EP300 | 0.62261 | 6329 | CSF1R | 0.62258 | 6330 | I.S. | 0.62255 |
| 6331 | I.S. | 0.62253 | 6332 | I.S. | 0.62252 | 6333 | — | 0.62249 |
| 6334 | RARA | 0.62249 | 6335 | I.S. | 0.62248 | 6336 | I.S. | 0.62248 |
| 6337 | I.S. | 0.62248 | 6338 | I.S. | 0.62248 | 6339 | I.S. | 0.62243 |
| 6340 | NTRK2 | 0.6224 | 6341 | SDHA | 0.62239 | 6342 | ADGRA2 | 0.62236 |
| 6343 | I.S. | 0.62236 | 6344 | I.S. | 0.62236 | 6345 | TSC1 | 0.62234 |
| 6346 | I.S. | 0.62225 | 6347 | RAD51D | 0.62223 | 6348 | I.S. | 0.62222 |
| 6349 | RICTOR | 0.6222 | 6350 | I.S. | 0.62216 | 6351 | KDM5A | 0.62216 |
| 6352 | I.S. | 0.62215 | 6353 | FGF3 | 0.62213 | 6354 | ERBB3 | 0.6221 |
| 6355 | I.S. | 0.62208 | 6356 | APC | 0.62207 | 6357 | I.S. | 0.62207 |
| 6358 | I.S. | 0.62207 | 6359 | I.S. | 0.62201 | 6360 | I.S. | 0.62196 |
| 6361 | I.S. | 0.62195 | 6362 | I.S. | 0.62189 | 6363 | I.S. | 0.62183 |
| 6364 | I.S. | 0.62181 | 6365 | I.S. | 0.62181 | 6366 | BRIP1 | 0.62181 |
| 6367 | RTEL1 | 0.62181 | 6368 | I.S. | 0.62179 | 6369 | I.S. | 0.62178 |
| 6370 | SPTA1 | 0.62178 | 6371 | I.S. | 0.62177 | 6372 | I.S. | 0.62177 |
| 6373 | I.S. | 0.62177 | 6374 | I.S. | 0.62174 | 6375 | I.S. | 0.62171 |
| 6376 | I.S. | 0.62171 | 6377 | I.S. | 0.62168 | 6378 | I.S. | 0.62167 |
| 6379 | I.S. | 0.62167 | 6380 | BCR | 0.62166 | 6381 | I.S. | 0.62165 |
| 6382 | I.S. | 0.62164 | 6383 | FLT3 | 0.62163 | 6384 | I.S. | 0.62162 |
| 6385 | CDAN1 | 0.62162 | 6386 | KMT2D | 0.6216 | 6387 | PIK3C2B | 0.62157 |
| 6388 | NOTCH1 | 0.6215 | 6389 | I.S. | 0.6215 | 6390 | I.S. | 0.62146 |
| 6391 | I.S. | 0.62146 | 6392 | I.S. | 0.62146 | 6393 | I.S. | 0.62138 |
| 6394 | I.S. | 0.62135 | 6395 | I.S. | 0.62135 | 6396 | ATR | 0.62134 |
| 6397 | XRCC3 | 0.62134 | 6398 | BRIP1 | 0.62128 | 6399 | MITF | 0.62126 |
| 6400 | I.S. | 0.62124 | 6401 | AXIN1 | 0.62119 | 6402 | CHD4 | 0.62116 |
| 6403 | NTRK3 | 0.62116 | 6404 | I.S. | 0.62114 | 6405 | FGFR2 | 0.62112 |
| 6406 | SEC23B | 0.62109 | 6407 | APC | 0.62107 | 6408 | I.S. | 0.62107 |
| 6409 | I.S. | 0.62105 | 6410 | TSHR | 0.62104 | 6411 | NLRP3 | 0.62092 |
| 6412 | CHEK1 | 0.62091 | 6413 | I.S. | 0.62091 | 6414 | PDGFRB | 0.62088 |
| 6415 | IGF2 | 0.62088 | 6416 | CD79A | 0.62088 | 6417 | I.S. | 0.62087 |
| 6418 | I.S. | 0.62083 | 6419 | I.S. | 0.62083 | 6420 | I.S. | 0.62081 |
| 6421 | RNF168 | 0.62077 | 6422 | I.S. | 0.62071 | 6423 | PRSS8 | 0.62071 |
| 6424 | I.S. | 0.62071 | 6425 | I.S. | 0.6207 | 6426 | I.S. | 0.62069 |
| 6427 | I.S. | 0.62069 | 6428 | RPTOR | 0.62067 | 6429 | I.S. | 0.62066 |
| 6430 | I.S. | 0.62065 | 6431 | I.S. | 0.62064 | 6432 | I.S. | 0.62059 |
| 6433 | I.S. | 0.62058 | 6434 | TSC2 | 0.62056 | 6435 | I.S. | 0.62055 |
| 6436 | I.S. | 0.62055 | 6437 | PIK3C2B | 0.62053 | 6438 | I.S. | 0.62049 |
| 6439 | PSTPIP1 | 0.62047 | 6440 | I.S. | 0.62046 | 6441 | I.S. | 0.62044 |
| 6442 | I.S. | 0.62042 | 6443 | I.S. | 0.62042 | 6444 | I.S. | 0.6204 |
| 6445 | I.S. | 0.6204 | 6446 | CREBBP | 0.62034 | 6447 | I.S. | 0.62034 |
| 6448 | I.S. | 0.62034 | 6449 | I.S. | 0.62034 | 6450 | I.S. | 0.62031 |
| 6451 | I.S. | 0.62031 | 6452 | I.S. | 0.62031 | 6453 | I.S. | 0.6203 |
| 6454 | PAX5 | 0.62029 | 6455 | I.S. | 0.62028 | 6456 | I.S. | 0.62028 |
| 6457 | I.S. | 0.62027 | 6458 | PTCH1 | 0.62027 | 6459 | I.S. | 0.62026 |
| 6460 | I.S. | 0.6202 | 6461 | MAGI2 | 0.6202 | 6462 | CDK12 | 0.62018 |
| 6463 | I.S. | 0.62017 | 6464 | I.S. | 0.62014 | 6465 | I.S. | 0.62013 |
| 6466 | I.S. | 0.6201 | 6467 | I.S. | 0.6201 | 6468 | I.S. | 0.6201 |
| 6469 | I.S. | 0.6201 | 6470 | KLHL6 | 0.62009 | 6471 | I.S. | 0.62009 |
| 6472 | EPHA3 | 0.62006 | 6473 | PRSS8 | 0.62001 | 6474 | I.S. | 0.61999 |
| 6475 | I.S. | 0.61999 | 6476 | SMC3 | 0.61993 | 6477 | SBF2 | 0.61993 |
| 6478 | I.S. | 0.61993 | 6479 | I.S. | 0.61991 | 6480 | I.S. | 0.61991 |
| 6481 | I.S. | 0.61985 | 6482 | I.S. | 0.61984 | 6483 | RICTOR | 0.61984 |
| 6484 | I.S. | 0.61981 | 6485 | MAGI2 | 0.61978 | 6486 | JAK2 | 0.61976 |
| 6487 | I.S. | 0.61974 | 6488 | BAP1 | 0.61973 | 6489 | I.S. | 0.61969 |
| 6490 | I.S. | 0.61968 | 6491 | SF3B1 | 0.61967 | 6492 | I.S. | 0.61963 |
| 6493 | NME1 | 0.61961 | 6494 | I.S. | 0.61961 | 6495 | PDGFRA | 0.61961 |
| 6496 | I.S. | 0.61955 | 6497 | I.S. | 0.61954 | 6498 | I.S. | 0.61951 |
| 6499 | I.S. | 0.61951 | 6500 | I.S. | 0.61949 | 6501 | I.S. | 0.61943 |
| 6502 | I.S. | 0.61934 | 6503 | I.S. | 0.61931 | 6504 | I.S. | 0.61931 |
| 6505 | I.S. | 0.61931 | 6506 | I.S. | 0.6193 | 6507 | I.S. | 0.61929 |
| 6508 | I.S. | 0.61929 | 6509 | I.S. | 0.61927 | 6510 | I.S. | 0.61924 |
| 6511 | NROB1 | 0.61923 | 6512 | I.S. | 0.61923 | 6513 | XRCC2 | 0.61921 |
| 6514 | I.S. | 0.61921 | 6515 | I.S. | 0.6192 | 6516 | I.S. | 0.61919 |
| 6517 | SMAD2 | 0.61919 | 6518 | HAX1 | 0.61917 | 6519 | I.S. | 0.61915 |
| 6520 | INHBA | 0.61913 | 6521 | PTCH1 | 0.61908 | 6522 | CCN6 | 0.61907 |
| 6523 | I.S. | 0.61906 | 6524 | I.S. | 0.61906 | 6525 | I.S. | 0.61905 |
| 6526 | KDM6A | 0.61904 | 6527 | ATR | 0.61904 | 6528 | I.S. | 0.61902 |
| 6529 | I.S. | 0.61901 | 6530 | I.S. | 0.619 | 6531 | I.S. | 0.619 |
| 6532 | I.S. | 0.619 | 6533 | I.S. | 0.61899 | 6534 | RTEL1 | 0.61898 |
| 6535 | ARID1B | 0.61898 | 6536 | NME1 | 0.61895 | 6537 | NTRK1 | 0.61893 |
| 6538 | I.S. | 0.61888 | 6539 | I.S. | 0.61888 | 6540 | CD79B | 0.61887 |
| 6541 | I.S. | 0.61886 | 6542 | GNAS | 0.61884 | 6543 | I.S. | 0.61883 |
| 6544 | I.S. | 0.61878 | 6545 | I.S. | 0.61878 | 6546 | I.S. | 0.61875 |
| 6547 | FH | 0.61875 | 6548 | I.S. | 0.61874 | 6549 | I.S. | 0.61872 |
| 6550 | I.S. | 0.61869 | 6551 | KEAP1 | 0.61869 | 6552 | AKT2 | 0.61869 |
| 6553 | AXIN1 | 0.61867 | 6554 | I.S. | 0.61864 | 6555 | I.S. | 0.61861 |
| 6556 | I.S. | 0.61857 | 6557 | GSK3B | 0.61857 | 6558 | I.S. | 0.61856 |
| 6559 | KMT2C | 0.61853 | 6560 | I.S. | 0.61853 | 6561 | I.S. | 0.6185 |
| 6562 | I.S. | 0.6185 | 6563 | I.S. | 0.61845 | 6564 | ATM | 0.61844 |
| 6565 | I.S. | 0.61843 | 6566 | I.S. | 0.61842 | 6567 | SPTA1 | 0.61835 |
| 6568 | MAGI2 | 0.61833 | 6569 | FANCA | 0.61833 | 6570 | I.S. | 0.61833 |
| 6571 | I.S. | 0.61829 | 6572 | I.S. | 0.61829 | 6573 | I.S. | 0.61828 |
| 6574 | PTCH1 | 0.61825 | 6575 | FANCA | 0.61825 | 6576 | NTRK1 | 0.61822 |
| 6577 | AKT1 | 0.61822 | 6578 | I.S. | 0.6182 | 6579 | EPCAM | 0.6182 |
| 6580 | I.S. | 0.6182 | 6581 | I.S. | 0.61806 | 6582 | I.S. | 0.61804 |
| 6583 | TET2 | 0.61803 | 6584 | TOP2A | 0.61798 | 6585 | SBF2 | 0.61797 |
| 6586 | I.S. | 0.61796 | 6587 | I.S. | 0.61796 | 6588 | I.S. | 0.61796 |
| 6589 | I.S. | 0.61796 | 6590 | I.S. | 0.61788 | 6591 | EED | 0.61788 |
| 6592 | I.S. | 0.61788 | 6593 | CDC73 | 0.61786 | 6594 | I.S. | 0.61782 |
| 6595 | I.S. | 0.61782 | 6596 | BCOR | 0.61779 | 6597 | POLE | 0.61779 |
| 6598 | I.S. | 0.61778 | 6599 | I.S. | 0.61777 | 6600 | ESR1 | 0.61776 |
| 6601 | I.S. | 0.61772 | 6602 | I.S. | 0.6177 | 6603 | I.S. | 0.61768 |
| 6604 | NTRK1 | 0.61765 | 6605 | I.S. | 0.6176 | 6606 | AURKC | 0.61759 |
| 6607 | SPTA1 | 0.61758 | 6608 | I.S. | 0.61754 | 6609 | FGFR2 | 0.6175 |
| 6610 | I.S. | 0.6175 | 6611 | I.S. | 0.61749 | 6612 | I.S. | 0.61749 |
| 6613 | I.S. | 0.61746 | 6614 | I.S. | 0.61741 | 6615 | BCL2 | 0.6174 |
| 6616 | I.S. | 0.61738 | 6617 | FGFR3 | 0.61735 | 6618 | I.S. | 0.6173 |
| 6619 | I.S. | 0.61729 | 6620 | I.S. | 0.61729 | 6621 | I.S. | 0.61724 |
| 6622 | CALR, | 0.61724 | 6623 | EGFR | 0.6172 | 6624 | I.S. | 0.61719 |
| | MIR6515 | | | | | | | |
| 6625 | I.S. | 0.61717 | 6626 | I.S. | 0.61717 | 6627 | I.S. | 0.61717 |
| 6628 | I.S. | 0.61717 | 6629 | I.S. | 0.61717 | 6630 | I.S. | 0.61713 |
| 6631 | I.S. | 0.61713 | 6632 | I.S. | 0.61713 | 6633 | TCF3 | 0.61711 |
| 6634 | I.S. | 0.61707 | 6635 | I.S. | 0.61705 | 6636 | I.S. | 0.61703 |
| 6637 | I.S. | 0.61702 | 6638 | I.S. | 0.61702 | 6639 | SDHB | 0.61699 |
| 6640 | RTEL1 | 0.61693 | 6641 | NOTCH2 | 0.61693 | 6642 | I.S. | 0.61692 |
| 6643 | I.S. | 0.61689 | 6644 | I.S. | 0.61688 | 6645 | I.S. | 0.61684 |
| 6646 | BLM | 0.61682 | 6647 | BRD4 | 0.61681 | 6648 | I.S. | 0.61681 |
| 6649 | I.S. | 0.61678 | 6650 | I.S. | 0.61675 | 6651 | I.S. | 0.61672 |
| 6652 | DICER1 | 0.61668 | 6653 | I.S. | 0.61667 | 6654 | TSC2 | 0.61664 |
| 6655 | I.S. | 0.61662 | 6656 | MPL | 0.61661 | 6657 | I.S. | 0.6166 |
| 6658 | I.S. | 0.61656 | 6659 | I.S. | 0.61652 | 6660 | I.S. | 0.61643 |
| 6661 | RAC1 | 0.61642 | 6662 | I.S. | 0.61639 | 6663 | MRE11 | 0.61638 |
| 6664 | I.S. | 0.61632 | 6665 | I.S. | 0.61631 | 6666 | KMT2B | 0.61629 |
| 6667 | RPTOR | 0.61628 | 6668 | I.S. | 0.61627 | 6669 | CHD2 | 0.61626 |
| 6670 | SF3B1 | 0.61625 | 6671 | I.S. | 0.61623 | 6672 | I.S. | 0.61623 |
| 6673 | I.S. | 0.61623 | 6674 | I.S. | 0.61621 | 6675 | I.S. | 0.61621 |
| 6676 | I.S. | 0.61615 | 6677 | I.S. | 0.61615 | 6678 | GNA11 | 0.6161 |
| 6679 | I.S. | 0.6161 | 6680 | I.S. | 0.61608 | 6681 | I.S. | 0.61606 |
| 6682 | CHD2 | 0.61604 | 6683 | I.S. | 0.61599 | 6684 | I.S. | 0.61598 |
| 6685 | RAD54L | 0.61597 | 6686 | MEF2B | 0.61593 | 6687 | GSKIP | 0.61592 |
| 6688 | I.S. | 0.61591 | 6689 | I.S. | 0.61591 | 6690 | I.S. | 0.61591 |
| 6691 | ERCC4 | 0.61589 | 6692 | IRS2 | 0.61584 | 6693 | I.S. | 0.61582 |
| 6694 | I.S. | 0.61579 | 6695 | KMT2C | 0.61576 | 6696 | I.S. | 0.61574 |
| 6697 | I.S. | 0.61573 | 6698 | I.S. | 0.61568 | 6699 | FLT1 | 0.61566 |
| 6700 | ROS1 | 0.61566 | 6701 | SETD2 | 0.61563 | 6702 | DDX41 | 0.6156 |
| 6703 | I.S. | 0.61558 | 6704 | LYST | 0.61556 | 6705 | AXL | 0.61552 |
| 6706 | MITF | 0.61549 | 6707 | RTEL1 | 0.61548 | 6708 | I.S. | 0.61539 |
| 6709 | I.S. | 0.61538 | 6710 | CHD2 | 0.61536 | 6711 | I.S. | 0.61536 |
| 6712 | I.S. | 0.61534 | 6713 | I.S. | 0.6153 | 6714 | SPTA1 | 0.61523 |
| 6715 | I.S. | 0.61522 | 6716 | — | 0.61521 | 6717 | I.S. | 0.6152 |
| 6718 | SMARCA4 | 0.61518 | 6719 | I.S. | 0.61518 | 6720 | I.S. | 0.61517 |
| 6721 | I.S. | 0.61511 | 6722 | I.S. | 0.61511 | 6723 | I.S. | 0.61507 |
| 6724 | MEFV | 0.61504 | 6725 | I.S. | 0.61503 | 6726 | I.S. | 0.615 |
| 6727 | IDH1 | 0.61489 | 6728 | I.S. | 0.61487 | 6729 | MSH6 | 0.61486 |
| 6730 | GLI1 | 0.61482 | 6731 | I.S. | 0.61481 | 6732 | PTCH1 | 0.6148 |
| 6733 | CIC | 0.6148 | 6734 | I.S. | 0.61479 | 6735 | I.S. | 0.61479 |
| 6736 | I.S. | 0.61476 | 6737 | I.S. | 0.61476 | 6738 | I.S. | 0.61475 |
| 6739 | I.S. | 0.61475 | 6740 | I.S. | 0.61473 | 6741 | I.S. | 0.61472 |
| 6742 | MTOR | 0.61467 | 6743 | I.S. | 0.61467 | 6744 | NF1 | 0.61467 |
| 6745 | I.S. | 0.61465 | 6746 | I.S. | 0.61464 | 6747 | NOTCH3 | 0.61458 |
| 6748 | I.S. | 0.61455 | 6749 | I.S. | 0.61455 | 6750 | I.S. | 0.61454 |
| 6751 | I.S. | 0.61448 | 6752 | STAT4 | 0.61447 | 6753 | DOT1L | 0.61447 |
| 6754 | I.S. | 0.61442 | 6755 | I.S. | 0.61438 | 6756 | IRS2 | 0.61438 |
| 6757 | I.S. | 0.61437 | 6758 | I.S. | 0.61435 | 6759 | I.S. | 0.61434 |
| 6760 | PRSS1 | 0.61423 | 6761 | I.S. | 0.61423 | 6762 | TCF3 | 0.6142 |
| 6763 | ABL2 | 0.6142 | 6764 | SPEN | 0.61414 | 6765 | I.S. | 0.61412 |
| 6766 | NF1 | 0.61408 | 6767 | I.S. | 0.61407 | 6768 | I.S. | 0.61406 |
| 6769 | I.S. | 0.61399 | 6770 | I.S. | 0.61399 | 6771 | FLT1 | 0.61399 |
| 6772 | VEGFA | 0.61398 | 6773 | — | 0.61397 | 6774 | I.S. | 0.61396 |
| 6775 | I.S. | 0.61396 | 6776 | TCF3 | 0.61394 | 6777 | NF1 | 0.61391 |
| 6778 | I.S. | 0.61389 | 6779 | I.S. | 0.61388 | 6780 | I.S. | 0.61385 |
| 6781 | I.S. | 0.61377 | 6782 | I.S. | 0.61375 | 6783 | I.S. | 0.61369 |
| 6784 | I.S. | 0.61368 | 6785 | KMT2D | 0.61364 | 6786 | JAK3 | 0.61361 |
| 6787 | CHD4 | 0.61361 | 6788 | I.S. | 0.6136 | 6789 | I.S. | 0.61356 |
| 6790 | DICER1 | 0.61352 | 6791 | PIK3R2 | 0.61346 | 6792 | I.S. | 0.61344 |
| 6793 | PALB2 | 0.61342 | 6794 | I.S. | 0.61341 | 6795 | I.S. | 0.61339 |
| 6796 | I.S. | 0.61339 | 6797 | I.S. | 0.61337 | 6798 | I.S. | 0.61336 |
| 6799 | I.S. | 0.61334 | 6800 | BLM | 0.61331 | 6801 | I.S. | 0.61327 |
| 6802 | I.S. | 0.61323 | 6803 | KDR | 0.61323 | 6804 | I.S. | 0.61322 |
| 6805 | I.S. | 0.61319 | 6806 | AKT3 | 0.61316 | 6807 | I.S. | 0.61316 |
| 6808 | I.S. | 0.61307 | 6809 | I.S. | 0.61305 | 6810 | SPTA1 | 0.61302 |
| 6811 | I.S. | 0.61301 | 6812 | I.S. | 0.613 | 6813 | I.S. | 0.61299 |
| 6814 | I.S. | 0.61298 | 6815 | DDX41 | 0.61296 | 6816 | I.S. | 0.61292 |
| 6817 | I.S. | 0.61289 | 6818 | I.S. | 0.61287 | 6819 | HNF1A | 0.61283 |
| 6820 | CTC1 | 0.61278 | 6821 | NOTCH1 | 0.61277 | 6822 | I.S. | 0.61273 |
| 6823 | CDKN2C | 0.61273 | 6824 | I.S. | 0.61269 | 6825 | I.S. | 0.61268 |
| 6826 | I.S. | 0.61267 | 6827 | I.S. | 0.61265 | 6828 | I.S. | 0.61265 |
| 6829 | I.S. | 0.61265 | 6830 | RICTOR | 0.61262 | 6831 | I.S. | 0.61262 |
| 6832 | I.S. | 0.61262 | 6833 | I.S. | 0.6126 | 6834 | LYST | 0.61259 |
| 6835 | ARFRP1 | 0.61259 | 6836 | EP300 | 0.61257 | 6837 | I.S. | 0.61256 |
| 6838 | I.S. | 0.61255 | 6839 | KDR | 0.61253 | 6840 | I.S. | 0.61243 |
| 6841 | I.S. | 0.61241 | 6842 | I.S. | 0.61239 | 6843 | I.S. | 0.61237 |
| 6844 | XPO1 | 0.6123 | 6845 | I.S. | 0.61227 | 6846 | I.S. | 0.61227 |
| 6847 | I.S. | 0.61227 | 6848 | PTPN11 | 0.61224 | 6849 | RICTOR | 0.61224 |
| 6850 | I.S. | 0.61218 | 6851 | I.S. | 0.61216 | 6852 | PRDM1 | 0.61215 |
| 6853 | POLE | 0.61215 | 6854 | ERCC4 | 0.61211 | 6855 | I.S. | 0.61206 |
| 6856 | I.S. | 0.61204 | 6857 | I.S. | 0.61204 | 6858 | I.S. | 0.61203 |
| 6859 | KEL | 0.61203 | 6860 | I.S. | 0.61199 | 6861 | I.S. | 0.61199 |
| 6862 | ZNF217 | 0.61194 | 6863 | I.S. | 0.61194 | 6864 | I.S. | 0.61194 |
| 6865 | MRE11 | 0.61194 | 6866 | I.S. | 0.61191 | 6867 | I.S. | 0.61189 |
| 6868 | SNCAIP | 0.61188 | 6869 | I.S. | 0.61186 | 6870 | I.S. | 0.61183 |
| 6871 | FLT4 | 0.6118 | 6872 | CDKN1A | 0.6118 | 6873 | NBN | 0.61174 |
| 6874 | RET | 0.61174 | 6875 | POLE | 0.61171 | 6876 | KMT2C | 0.6117 |
| 6877 | MLH1 | 0.6117 | 6878 | I.S. | 0.61169 | 6879 | I.S. | 0.61169 |
| 6880 | I.S. | 0.61166 | 6881 | I.S. | 0.61162 | 6882 | — | 0.61159 |
| 6883 | NF2 | 0.61159 | 6884 | I.S. | 0.61158 | 6885 | I.S. | 0.61158 |
| 6886 | I.S. | 0.61153 | 6887 | TOP2A | 0.6115 | 6888 | I.S. | 0.61149 |
| 6889 | ZNF217 | 0.61141 | 6890 | CD79A | 0.61141 | 6891 | I.S. | 0.61139 |
| 6892 | I.S. | 0.61137 | 6893 | I.S. | 0.61137 | 6894 | I.S. | 0.61137 |
| 6895 | I.S. | 0.61136 | 6896 | I.S. | 0.61133 | 6897 | NPM1 | 0.61133 |
| 6898 | I.S. | 0.61132 | 6899 | I.S. | 0.61127 | 6900 | I.S. | 0.61126 |
| 6901 | I.S. | 0.61125 | 6902 | CDH1 | 0.61125 | 6903 | I.S. | 0.61124 |
| 6904 | I.S. | 0.61124 | 6905 | I.S. | 0.61122 | 6906 | ZNF217 | 0.61118 |
| 6907 | CHD4 | 0.61117 | 6908 | LYST | 0.61111 | 6909 | CUX1 | 0.61109 |
| 6910 | FANCA | 0.61108 | 6911 | I.S. | 0.61104 | 6912 | I.S. | 0.61104 |
| 6913 | SNCAIP | 0.61102 | 6914 | I.S. | 0.61101 | 6915 | I.S. | 0.61099 |
| 6916 | I.S. | 0.61097 | 6917 | I.S. | 0.61097 | 6918 | I.S. | 0.61093 |
| 6919 | I.S. | 0.61093 | 6920 | TOP2A | 0.61093 | 6921 | I.S. | 0.6109 |
| 6922 | I.S. | 0.61089 | 6923 | TP53 | 0.61088 | 6924 | I.S. | 0.61085 |
| 6925 | FGFR4 | 0.61082 | 6926 | TNFAIP3 | 0.61078 | 6927 | I.S. | 0.61072 |
| 6928 | I.S. | 0.61071 | 6929 | I.S. | 0.61068 | 6930 | I.S. | 0.61068 |
| 6931 | I.S. | 0.61068 | 6932 | LRP1B | 0.61067 | 6933 | FANCG | 0.61064 |
| 6934 | I.S. | 0.61064 | 6935 | I.S. | 0.61061 | 6936 | DDX41 | 0.61057 |
| 6937 | I.S. | 0.61054 | 6938 | I.S. | 0.61054 | 6939 | FANCI | 0.6105 |
| 6940 | NTRK1 | 0.61048 | 6941 | I.S. | 0.61044 | 6942 | PIK3CG | 0.6104 |
| 6943 | I.S. | 0.61038 | 6944 | I.S. | 0.61035 | 6945 | ATR | 0.61034 |
| 6946 | PRKCI | 0.61033 | 6947 | PRKDC | 0.61026 | 6948 | I.S. | 0.61019 |
| 6949 | I.S. | 0.61019 | 6950 | I.S. | 0.61016 | 6951 | I.S. | 0.61016 |
| 6952 | I.S. | 0.61009 | 6953 | ERBB4 | 0.61006 | 6954 | SETD2 | 0.61006 |
| 6955 | I.S. | 0.61002 | 6956 | I.S. | 0.61002 | 6957 | KDM5C | 0.61001 |
| 6958 | I.S. | 0.60998 | 6959 | I.S. | 0.60998 | 6960 | SMAD9 | 0.60996 |
| 6961 | CHD2 | 0.60994 | 6962 | BRCA1 | 0.60993 | 6963 | RTEL1 | 0.60989 |
| 6964 | I.S. | 0.60987 | 6965 | I.S. | 0.60985 | 6966 | I.S. | 0.60985 |
| 6967 | I.S. | 0.6098 | 6968 | I.S. | 0.6098 | 6969 | AKT3 | 0.60978 |
| 6970 | APC | 0.60975 | 6971 | CDAN1 | 0.60974 | 6972 | KIT | 0.60974 |
| 6973 | DDX11 | 0.60971 | 6974 | I.S. | 0.60963 | 6975 | KLLN | 0.6096 |
| 6976 | I.S. | 0.6096 | 6977 | I.S. | 0.60956 | 6978 | I.S. | 0.60954 |
| 6979 | I.S. | 0.60949 | 6980 | I.S. | 0.60947 | 6981 | I.S. | 0.60944 |
| 6982 | I.S. | 0.60939 | 6983 | I.S. | 0.60939 | 6984 | SNCAIP | 0.60938 |
| 6985 | I.S. | 0.60936 | 6986 | RBBP6 | 0.60932 | 6987 | I.S. | 0.6093 |
| 6988 | I.S. | 0.60927 | 6989 | KDR | 0.60924 | 6990 | I.S. | 0.60923 |
| 6991 | I.S. | 0.60923 | 6992 | I.S. | 0.6092 | 6993 | I.S. | 0.6092 |
| 6994 | I.S. | 0.60917 | 6995 | I.S. | 0.60917 | 6996 | I.S. | 0.60916 |
| 6997 | KMT2A | 0.60916 | 6998 | I.S. | 0.60911 | 6999 | AKT3 | 0.60909 |
| 7000 | SUZ12 | 0.60906 | 7001 | SOX9 | 0.60906 | 7002 | I.S. | 0.60901 |
| 7003 | PRKDC | 0.60901 | 7004 | I.S. | 0.609 | 7005 | I.S. | 0.60899 |
| 7006 | I.S. | 0.60899 | 7007 | I.S. | 0.60899 | 7008 | I.S. | 0.60897 |
| 7009 | PREX2 | 0.60895 | 7010 | DDX11 | 0.60893 | 7011 | STK11 | 0.60891 |
| 7012 | I.S. | 0.6089 | 7013 | I.S. | 0.60888 | 7014 | I.S. | 0.60887 |
| 7015 | FGFR4 | 0.60886 | 7016 | I.S. | 0.60886 | 7017 | MYCL | 0.6088 |
| 7018 | NOTCH2 | 0.60875 | 7019 | SUZ12 | 0.60874 | 7020 | MAPK1 | 0.60873 |
| 7021 | I.S. | 0.60873 | 7022 | I.S. | 0.60873 | 7023 | I.S. | 0.6087 |
| 7024 | I.S. | 0.6087 | 7025 | I.S. | 0.60867 | 7026 | I.S. | 0.60863 |
| 7027 | CIC | 0.60862 | 7028 | CSF3R | 0.6086 | 7029 | I.S. | 0.6086 |
| 7030 | ATM | 0.60859 | 7031 | I.S. | 0.60859 | 7032 | JAK3 | 0.60856 |
| 7033 | — | 0.60853 | 7034 | AURKB | 0.6085 | 7035 | MAP2K2 | 0.60846 |
| 7036 | I.S. | 0.60844 | 7037 | I.S. | 0.6084 | 7038 | I.S. | 0.6084 |
| 7039 | I.S. | 0.60835 | 7040 | LMO1 | 0.60835 | 7041 | EPAS1 | 0.60833 |
| 7042 | I.S. | 0.60829 | 7043 | I.S. | 0.60828 | 7044 | I.S. | 0.6082 |
| 7045 | I.S. | 0.60816 | 7046 | ATM | 0.60816 | 7047 | PIK3C2B | 0.60815 |
| 7048 | RICTOR | 0.60814 | 7049 | I.S. | 0.60813 | 7050 | I.S. | 0.60813 |
| 7051 | I.S. | 0.60813 | 7052 | I.S. | 0.60812 | 7053 | I.S. | 0.60811 |
| 7054 | I.S. | 0.60811 | 7055 | I.S. | 0.6081 | 7056 | IGF1R | 0.60808 |
| 7057 | GNAS | 0.60808 | 7058 | SDHB | 0.60808 | 7059 | CREBBP | 0.60808 |
| 7060 | RICTOR | 0.60799 | 7061 | I.S. | 0.60798 | 7062 | IKZF3 | 0.60796 |
| 7063 | I.S. | 0.60794 | 7064 | TERF2 | 0.60791 | 7065 | I.S. | 0.6079 |
| 7066 | I.S. | 0.60789 | 7067 | ETV6 | 0.60787 | 7068 | I.S. | 0.60766 |
| 7069 | LMO1 | 0.60763 | 7070 | I.S. | 0.6076 | 7071 | I.S. | 0.60758 |
| 7072 | I.S. | 0.60758 | 7073 | I.S. | 0.60758 | 7074 | I.S. | 0.60756 |
| 7075 | IDH2 | 0.60754 | 7076 | PBRM1 | 0.60752 | 7077 | CHD2 | 0.60749 |
| 7078 | I.S. | 0.60747 | 7079 | AURKB | 0.60746 | 7080 | I.S. | 0.60742 |
| 7081 | I.S. | 0.60741 | 7082 | CDK8 | 0.6074 | 7083 | PSTPIP1 | 0.6074 |
| 7084 | SETD2 | 0.60739 | 7085 | I.S. | 0.60734 | 7086 | EZH2 | 0.60731 |
| 7087 | I.S. | 0.60728 | 7088 | CUX1 | 0.60722 | 7089 | I.S. | 0.60722 |
| 7090 | I.S. | 0.60718 | 7091 | I.S. | 0.60717 | 7092 | CSF3R | 0.60717 |
| 7093 | I.S. | 0.60714 | 7094 | LRP1B | 0.60713 | 7095 | DICER1 | 0.60713 |
| 7096 | I.S. | 0.60712 | 7097 | I.S. | 0.60712 | 7098 | I.S. | 0.60709 |
| 7099 | I.S. | 0.60709 | 7100 | IKZF1 | 0.60707 | 7101 | I.S. | 0.60701 |
| 7102 | GLI1 | 0.60693 | 7103 | I.S. | 0.60692 | 7104 | I.S. | 0.60691 |
| 7105 | NF1 | 0.60689 | 7106 | ATG2B | 0.60689 | 7107 | ROS1 | 0.60684 |
| 7108 | I.S. | 0.60682 | 7109 | I.S. | 0.60675 | 7110 | SMAD4 | 0.60675 |
| 7111 | I.S. | 0.60674 | 7112 | I.S. | 0.60672 | 7113 | I.S. | 0.60671 |
| 7114 | I.S. | 0.6067 | 7115 | ARID1A | 0.60669 | 7116 | I.S. | 0.60668 |
| 7117 | I.S. | 0.60665 | 7118 | I.S. | 0.60664 | 7119 | I.S. | 0.60663 |
| 7120 | SUFU | 0.6066 | 7121 | I.S. | 0.60659 | 7122 | ZNF703 | 0.60659 |
| 7123 | MYD88 | 0.60659 | 7124 | GRIN2A | 0.60659 | 7125 | MAP2K1 | 0.60657 |
| 7126 | I.S. | 0.60652 | 7127 | I.S. | 0.60651 | 7128 | I.S. | 0.60651 |
| 7129 | SEC23B | 0.60649 | 7130 | HGF | 0.60648 | 7131 | TSC2 | 0.60647 |
| 7132 | I.S. | 0.60645 | 7133 | ALK | 0.60642 | 7134 | I.S. | 0.60642 |
| 7135 | I.S. | 0.60641 | 7136 | CHD2 | 0.6064 | 7137 | KMT2D | 0.60639 |
| 7138 | I.S. | 0.60636 | 7139 | CSF1R | 0.6063 | 7140 | I.S. | 0.60627 |
| 7141 | POLE | 0.60624 | 7142 | I.S. | 0.60611 | 7143 | AKT1 | 0.6061 |
| 7144 | SPEN | 0.60609 | 7145 | I.S. | 0.60608 | 7146 | BARD1 | 0.60606 |
| 7147 | I.S. | 0.60605 | 7148 | I.S. | 0.60602 | 7149 | GLI1 | 0.60602 |
| 7150 | I.S. | 0.60601 | 7151 | I.S. | 0.606 | 7152 | I.S. | 0.60599 |
| 7153 | RAD50 | 0.60597 | 7154 | NSD1 | 0.60595 | 7155 | SPTA1 | 0.60594 |
| 7156 | I.S. | 0.60594 | 7157 | I.S. | 0.60585 | 7158 | FOXP1 | 0.60582 |
| 7159 | I.S. | 0.60579 | 7160 | I.S. | 0.60578 | 7161 | I.S. | 0.60577 |
| 7162 | I.S. | 0.60575 | 7163 | AKT1 | 0.60574 | 7164 | I.S. | 0.6057 |
| 7165 | I.S. | 0.6057 | 7166 | I.S. | 0.60568 | 7167 | I.S. | 0.60566 |
| 7168 | I.S. | 0.60563 | 7169 | EPHA7 | 0.60562 | 7170 | I.S. | 0.60561 |
| 7171 | I.S. | 0.60555 | 7172 | I.S. | 0.60553 | 7173 | GABRA6 | 0.60553 |
| 7174 | I.S. | 0.6055 | 7175 | I.S. | 0.60549 | 7176 | I.S. | 0.60547 |
| 7177 | ARID1A | 0.60546 | 7178 | I.S. | 0.60543 | 7179 | I.S. | 0.60542 |
| 7180 | I.S. | 0.60537 | 7181 | I.S. | 0.60537 | 7182 | I.S. | 0.60534 |
| 7183 | EGFR | 0.60529 | 7184 | I.S. | 0.60528 | 7185 | I.S. | 0.60523 |
| 7186 | I.S. | 0.6052 | 7187 | CCNE1 | 0.60518 | 7188 | MVK | 0.60505 |
| 7189 | ALK | 0.605 | 7190 | BCL2 | 0.60497 | 7191 | CTC1 | 0.60496 |
| 7192 | PLCG1 | 0.60496 | 7193 | I.S. | 0.60494 | 7194 | I.S. | 0.60493 |
| 7195 | I.S. | 0.60492 | 7196 | I.S. | 0.60487 | 7197 | ZNF217 | 0.60479 |
| 7198 | CDK4 | 0.60478 | 7199 | RUNX1 | 0.60476 | 7200 | ATM | 0.60473 |
| 7201 | I.S. | 0.60471 | 7202 | I.S. | 0.60468 | 7203 | TET2 | 0.60466 |
| 7204 | I.S. | 0.60466 | 7205 | I.S. | 0.60457 | 7206 | I.S. | 0.60454 |
| 7207 | I.S. | 0.60448 | 7208 | ABL2 | 0.60442 | 7209 | I.S. | 0.60442 |
| 7210 | CTNNB1 | 0.60442 | 7211 | CBL | 0.60441 | 7212 | KIT | 0.60435 |
| 7213 | I.S. | 0.60434 | 7214 | I.S. | 0.60434 | 7215 | MAP3K14 | 0.60431 |
| 7216 | LPIN2 | 0.60431 | 7217 | I.S. | 0.60428 | 7218 | RAD51C | 0.60426 |
| 7219 | ADGRA2 | 0.60425 | 7220 | I.S. | 0.60425 | 7221 | I.S. | 0.60425 |
| 7222 | APC | 0.60422 | 7223 | I.S. | 0.60421 | 7224 | PRDM1 | 0.60421 |
| 7225 | I.S. | 0.6042 | 7226 | I.S. | 0.60414 | 7227 | ATR | 0.60413 |
| 7228 | I.S. | 0.60412 | 7229 | I.S. | 0.6041 | 7230 | JAK1 | 0.60404 |
| 7231 | PDGFRB | 0.60402 | 7232 | FUBP1 | 0.60402 | 7233 | BCL6 | 0.60401 |
| 7234 | I.S. | 0.604 | 7235 | I.S. | 0.604 | 7236 | I.S. | 0.604 |
| 7237 | I.S. | 0.60399 | 7238 | PAX5 | 0.60399 | 7239 | SFTA3, | 0.60396 |
| | | | | | | | NKX2-1 | |
| 7240 | SETBP1 | 0.60396 | 7241 | CHD2 | 0.60394 | 7242 | I.S. | 0.60393 |
| 7243 | DICER1 | 0.60393 | 7244 | MTOR | 0.60389 | 7245 | I.S. | 0.60389 |
| 7246 | CHD2 | 0.60385 | 7247 | I.S. | 0.60381 | 7248 | FANCM | 0.6038 |
| 7249 | I.S. | 0.60372 | 7250 | I.S. | 0.60367 | 7251 | I.S. | 0.6036 |
| 7252 | WT1 | 0.60345 | 7253 | EPHA3 | 0.60345 | 7254 | TCIRG1 | 0.60344 |
| 7255 | I.S. | 0.60342 | 7256 | ATM | 0.60342 | 7257 | I.S. | 0.60339 |
| 7258 | LYST | 0.60336 | 7259 | SMARCB1 | 0.60328 | 7260 | I.S. | 0.60326 |
| 7261 | I.S. | 0.60321 | 7262 | NSD1 | 0.60316 | 7263 | I.S. | 0.60313 |
| 7264 | I.S. | 0.60309 | 7265 | I.S. | 0.60309 | 7266 | I.S. | 0.60297 |
| 7267 | BRD4 | 0.60297 | 7268 | TET2 | 0.60297 | 7269 | I.S. | 0.60296 |
| 7270 | I.S. | 0.60296 | 7271 | PRSS8 | 0.60295 | 7272 | RICTOR | 0.60285 |
| 7273 | I.S. | 0.60276 | 7274 | I.S. | 0.60273 | 7275 | I.S. | 0.60272 |
| 7276 | RET | 0.60272 | 7277 | I.S. | 0.60268 | 7278 | I.S. | 0.60266 |
| 7279 | I.S. | 0.60265 | 7280 | I.S. | 0.60264 | 7281 | I.S. | 0.60264 |
| 7282 | KLF1 | 0.60262 | 7283 | KMT2B | 0.60255 | 7284 | I.S. | 0.6025 |
| 7285 | I.S. | 0.60249 | 7286 | I.S. | 0.60247 | 7287 | I.S. | 0.60247 |
| 7288 | FANCM | 0.6024 | 7289 | I.S. | 0.60231 | 7290 | I.S. | 0.60231 |
| 7291 | I.S. | 0.60222 | 7292 | I.S. | 0.60222 | 7293 | GLI2 | 0.6022 |
| 7294 | RPTOR | 0.6022 | 7295 | I.S. | 0.60219 | 7296 | I.S. | 0.60213 |
| 7297 | I.S. | 0.60211 | 7298 | RB1 | 0.60211 | 7299 | MAP3K1 | 0.60208 |
| 7300 | I.S. | 0.60207 | 7301 | I.S. | 0.60202 | 7302 | I.S. | 0.60201 |
| 7303 | I.S. | 0.60195 | 7304 | TGFBR2 | 0.60188 | 7305 | TNFAIP3 | 0.60188 |
| 7306 | I.S. | 0.60186 | 7307 | GABRA6 | 0.60184 | 7308 | ERBB4 | 0.60182 |
| 7309 | I.S. | 0.60175 | 7310 | I.S. | 0.60171 | 7311 | I.S. | 0.60167 |
| 7312 | CDK12 | 0.60163 | 7313 | NROB1 | 0.60162 | 7314 | I.S. | 0.6016 |
| 7315 | I.S. | 0.6016 | 7316 | PRDM1 | 0.60157 | 7317 | I.S. | 0.60154 |
| 7318 | KMT2B | 0.60154 | 7319 | ATG2B | 0.60152 | 7320 | I.S. | 0.6015 |
| 7321 | I.S. | 0.60149 | 7322 | SBDS | 0.60147 | 7323 | BRCA1 | 0.60146 |
| 7324 | ANKRD26 | 0.60139 | 7325 | I.S. | 0.60137 | 7326 | SLIT2 | 0.60137 |
| 7327 | I.S. | 0.60132 | 7328 | I.S. | 0.60132 | 7329 | PDGFRB | 0.60128 |
| 7330 | EPAS1 | 0.60128 | 7331 | WT1 | 0.60121 | 7332 | ADGRA2 | 0.6012 |
| 7333 | KAT6A | 0.60119 | 7334 | I.S. | 0.60115 | 7335 | CTC1 | 0.60115 |
| 7336 | SF3B1 | 0.60113 | 7337 | I.S. | 0.60113 | 7338 | I.S. | 0.60112 |
| 7339 | NOTCH3 | 0.60112 | 7340 | I.S. | 0.60105 | 7341 | I.S. | 0.60104 |
| 7342 | FGFR3 | 0.60104 | 7343 | KIT | 0.60104 | 7344 | CDH1 | 0.60104 |
| 7345 | I.S. | 0.60104 | 7346 | I.S. | 0.60101 | 7347 | I.S. | 0.60099 |
| 7348 | CSF3R | 0.60098 | 7349 | I.S. | 0.60096 | 7350 | I.S. | 0.60095 |
| 7351 | I.S. | 0.60083 | 7352 | I.S. | 0.60083 | 7353 | I.S. | 0.60078 |
| 7354 | I.S. | 0.60075 | 7355 | I.S. | 0.60074 | 7356 | I.S. | 0.60071 |
| 7357 | I.S. | 0.60066 | 7358 | JAK2 | 0.60063 | 7359 | I.S. | 0.60063 |
| 7360 | NOTCH1, | 0.60059 | 7361 | I.S. | 0.60056 | 7362 | I.S. | 0.60049 |
| | MIR4673 | | | | | | | |
| 7363 | I.S. | 0.60048 | 7364 | I.S. | 0.60047 | 7365 | I.S. | 0.60038 |
| 7366 | SPTA1 | 0.60036 | 7367 | CHD2 | 0.60036 | 7368 | I.S. | 0.60033 |
| 7369 | SMAD4 | 0.6003 | 7370 | I.S. | 0.6003 | 7371 | HGF | 0.60029 |
| 7372 | I.S. | 0.60027 | 7373 | I.S. | 0.60021 | 7374 | GABRA6 | 0.60016 |
| 7375 | I.S. | 0.60014 | 7376 | I.S. | 0.60014 | 7377 | I.S. | 0.60013 |
| 7378 | PREX2 | 0.6 | 7379 | GLI2 | 0.6 | 7380 | I.S. | 0.59997 |
| 7381 | I.S. | 0.59997 | 7382 | I.S. | 0.59993 | 7383 | ZBTB2 | 0.59989 |
| 7384 | JAK1 | 0.59988 | 7385 | I.S. | 0.59974 | 7386 | SMARCB1 | 0.59973 |
| 7387 | I.S. | 0.59969 | 7388 | I.S. | 0.59967 | 7389 | I.S. | 0.59959 |
| 7390 | I.S. | 0.59956 | 7391 | I.S. | 0.59955 | 7392 | I.S. | 0.59954 |
| 7393 | MSH6 | 0.59953 | 7394 | I.S. | 0.59951 | 7395 | I.S. | 0.5995 |
| 7396 | I.S. | 0.59947 | 7397 | FANCE | 0.59944 | 7398 | I.S. | 0.59943 |
| 7399 | I.S. | 0.59933 | 7400 | I.S. | 0.59932 | 7401 | I.S. | 0.59931 |
| 7402 | I.S. | 0.5993 | 7403 | I.S. | 0.59928 | 7404 | I.S. | 0.59921 |
| 7405 | PIM1 | 0.5992 | 7406 | I.S. | 0.59917 | 7407 | I.S. | 0.59916 |
| 7408 | I.S. | 0.59916 | 7409 | I.S. | 0.59914 | 7410 | JAK3 | 0.59914 |
| 7411 | KMT2A | 0.59912 | 7412 | I.S. | 0.59909 | 7413 | MAGI2 | 0.59907 |
| 7414 | BCORL1 | 0.59906 | 7415 | APC | 0.59904 | 7416 | FGF19 | 0.59904 |
| 7417 | I.S. | 0.59902 | 7418 | I.S. | 0.59902 | 7419 | I.S. | 0.59899 |
| 7420 | I.S. | 0.59899 | 7421 | BRCA1 | 0.59899 | 7422 | KAT6A | 0.59897 |
| 7423 | JAK2 | 0.59893 | 7424 | I.S. | 0.5989 | 7425 | GLI2 | 0.5989 |
| 7426 | I.S. | 0.59888 | 7427 | FANCB | 0.59887 | 7428 | I.S. | 0.59886 |
| 7429 | I.S. | 0.59884 | 7430 | MYD88 | 0.59878 | 7431 | I.S. | 0.59878 |
| 7432 | I.S. | 0.5987 | 7433 | I.S. | 0.59869 | 7434 | GID4 | 0.59867 |
| 7435 | KMT2A | 0.59866 | 7436 | KMT2D | 0.59866 | 7437 | I.S. | 0.59864 |
| 7438 | I.S. | 0.59864 | 7439 | I.S. | 0.59864 | 7440 | I.S. | 0.59863 |
| 7441 | I.S. | 0.59862 | 7442 | I.S. | 0.59861 | 7443 | ZNF703 | 0.59861 |
| 7444 | I.S. | 0.59858 | 7445 | I.S. | 0.59856 | 7446 | CREBBP | 0.59851 |
| 7447 | I.S. | 0.5985 | 7448 | LYST | 0.59846 | 7449 | I.S. | 0.59846 |
| 7450 | I.S. | 0.59843 | 7451 | I.S. | 0.5984 | 7452 | SPTA1 | 0.59836 |
| 7453 | I.S. | 0.59835 | 7454 | I.S. | 0.59835 | 7455 | PBRM1 | 0.59835 |
| 7456 | I.S. | 0.59825 | 7457 | IGF1R | 0.59825 | 7458 | I.S. | 0.5982 |
| 7459 | I.S. | 0.5982 | 7460 | I.S. | 0.59814 | 7461 | TNFAIP3 | 0.59813 |
| 7462 | I.S. | 0.59809 | 7463 | CREBBP | 0.59808 | 7464 | CD79B | 0.59808 |
| 7465 | MET | 0.59804 | 7466 | GNAS | 0.59804 | 7467 | ERCC4 | 0.59801 |
| 7468 | — | 0.59798 | 7469 | I.S. | 0.59795 | 7470 | KMT2D | 0.59792 |
| 7471 | POLE | 0.59784 | 7472 | I.S. | 0.59784 | 7473 | ATM | 0.59778 |
| 7474 | I.S. | 0.59777 | 7475 | I.S. | 0.59776 | 7476 | FLT4 | 0.59775 |
| 7477 | I.S. | 0.59774 | 7478 | I.S. | 0.59774 | 7479 | FANCI | 0.59768 |
| 7480 | ERBB2 | 0.59768 | 7481 | I.S. | 0.59767 | 7482 | I.S. | 0.59763 |
| 7483 | ALK | 0.59762 | 7484 | ERBB3 | 0.59761 | 7485 | I.S. | 0.59757 |
| 7486 | JAK3 | 0.59755 | 7487 | I.S. | 0.59754 | 7488 | I.S. | 0.59753 |
| 7489 | I.S. | 0.59751 | 7490 | IRS2 | 0.59743 | 7491 | CHEK2 | 0.59742 |
| 7492 | I.S. | 0.59741 | 7493 | I.S. | 0.5974 | 7494 | RET | 0.59735 |
| 7495 | SPEN | 0.59734 | 7496 | BCL6 | 0.5973 | 7497 | KMT2A | 0.59728 |
| 7498 | I.S. | 0.59725 | 7499 | I.S. | 0.59721 | 7500 | TP53 | 0.59718 |
| 7501 | NUP93 | 0.59715 | 7502 | I.S. | 0.59715 | 7503 | I.S. | 0.59715 |
| 7504 | I.S. | 0.59715 | 7505 | PIK3CA | 0.59712 | 7506 | I.S. | 0.59712 |
| 7507 | I.S. | 0.59704 | 7508 | I.S. | 0.59702 | 7509 | I.S. | 0.59698 |
| 7510 | I.S. | 0.59697 | 7511 | I.S. | 0.59692 | 7512 | I.S. | 0.59688 |
| 7513 | I.S. | 0.59688 | 7514 | I.S. | 0.59687 | 7515 | I.S. | 0.59683 |
| 7516 | I.S. | 0.59682 | 7517 | I.S. | 0.5968 | 7518 | GID4 | 0.59676 |
| 7519 | I.S. | 0.59674 | 7520 | ARID1A | 0.59673 | 7521 | RUNX1T1 | 0.59667 |
| 7522 | I.S. | 0.59667 | 7523 | I.S. | 0.59667 | 7524 | MDM2 | 0.59667 |
| 7525 | I.S. | 0.59666 | 7526 | I.S. | 0.59665 | 7527 | FRS2 | 0.59655 |
| 7528 | CHD4 | 0.59652 | 7529 | GNA13 | 0.5965 | 7530 | JAK3 | 0.59647 |
| 7531 | I.S. | 0.59646 | 7532 | KMT2B | 0.59645 | 7533 | SETD2 | 0.59634 |
| 7534 | BRCA1 | 0.59631 | 7535 | CARD11 | 0.59624 | 7536 | RPTOR | 0.59623 |
| 7537 | CTNNB1 | 0.59618 | 7538 | DNM2 | 0.59618 | 7539 | FLT1 | 0.59614 |
| 7540 | I.S. | 0.59609 | 7541 | PIK3R1 | 0.59608 | 7542 | TSC2 | 0.59608 |
| 7543 | I.S. | 0.596 | 7544 | I.S. | 0.596 | 7545 | I.S. | 0.59598 |
| 7546 | I.S. | 0.59598 | 7547 | I.S. | 0.59597 | 7548 | SMAD2 | 0.59594 |
| 7549 | I.S. | 0.59591 | 7550 | SMO | 0.5959 | 7551 | NSD2 | 0.59589 |
| 7552 | I.S. | 0.59584 | 7553 | I.S. | 0.59582 | 7554 | I.S. | 0.59578 |
| 7555 | STK11 | 0.59578 | 7556 | GNAS | 0.59573 | 7557 | I.S. | 0.5957 |
| 7558 | I.S. | 0.5957 | 7559 | RICTOR | 0.59566 | 7560 | I.S. | 0.59564 |
| 7561 | DDX11 | 0.59558 | 7562 | SLX4 | 0.59549 | 7563 | I.S. | 0.59545 |
| 7564 | I.S. | 0.59538 | 7565 | KMT2A | 0.59532 | 7566 | I.S. | 0.59527 |
| 7567 | KDM5C, | 0.59527 | 7568 | I.S. | 0.59525 | 7569 | MDM4 | 0.59519 |
| | MIR6894, | | | | | | | |
| | KDM5C | | | | | | | |
| 7570 | TBX3 | 0.59519 | 7571 | I.S. | 0.59516 | 7572 | I.S. | 0.59516 |
| 7573 | I.S. | 0.59513 | 7574 | I.S. | 0.59498 | 7575 | I.S. | 0.59492 |
| 7576 | MAP3K14 | 0.59487 | 7577 | FRS2 | 0.59483 | 7578 | I.S. | 0.5948 |
| 7579 | CUX1 | 0.59475 | 7580 | MAP3K1 | 0.59466 | 7581 | I.S. | 0.59458 |
| 7582 | I.S. | 0.59455 | 7583 | JAK1 | 0.59454 | 7584 | I.S. | 0.59451 |
| 7585 | I.S. | 0.59444 | 7586 | KIT | 0.59443 | 7587 | BCR | 0.59442 |
| 7588 | FAT1 | 0.59439 | 7589 | LRP1B | 0.59438 | 7590 | I.S. | 0.59438 |
| 7591 | RARA | 0.59434 | 7592 | TRAF3 | 0.5943 | 7593 | I.S. | 0.59426 |
| 7594 | I.S. | 0.59424 | 7595 | SAMD9L | 0.59424 | 7596 | I.S. | 0.59422 |
| 7597 | KDR | 0.59417 | 7598 | I.S. | 0.59417 | 7599 | CHD4 | 0.59412 |
| 7600 | RAD50, | 0.59408 | 7601 | GNAS | 0.59406 | 7602 | I.S. | 0.59386 |
| | TH2LCRR | | | | | | | |
| 7603 | I.S. | 0.59386 | 7604 | EZH2 | 0.59383 | 7605 | I.S. | 0.59379 |
| 7606 | LYN | 0.59379 | 7607 | I.S. | 0.59375 | 7608 | LRP1B | 0.59373 |
| 7609 | EGFR | 0.59373 | 7610 | I.S. | 0.59373 | 7611 | FLT4 | 0.59365 |
| 7612 | MEN1 | 0.59365 | 7613 | I.S. | 0.59364 | 7614 | I.S. | 0.59364 |
| 7615 | I.S. | 0.59364 | 7616 | I.S. | 0.59362 | 7617 | RET | 0.59361 |
| 7618 | RHEB | 0.59359 | 7619 | KDM5C | 0.59359 | 7620 | I.S. | 0.59359 |
| 7621 | GABRA6 | 0.59358 | 7622 | I.S. | 0.59352 | 7623 | MET | 0.5935 |
| 7624 | I.S. | 0.5935 | 7625 | MTOR | 0.5935 | 7626 | I.S. | 0.5935 |
| 7627 | I.S. | 0.59349 | 7628 | KMT2D | 0.59346 | 7629 | I.S. | 0.59345 |
| 7630 | I.S. | 0.59332 | 7631 | I.S. | 0.5933 | 7632 | JAK3 | 0.5933 |
| 7633 | I.S. | 0.59329 | 7634 | SLX4 | 0.59329 | 7635 | I.S. | 0.59325 |
| 7636 | I.S. | 0.59324 | 7637 | I.S. | 0.59321 | 7638 | KAT6A | 0.59321 |
| 7639 | I.S. | 0.5932 | 7640 | DDX11 | 0.59318 | 7641 | RAD21 | 0.59316 |
| 7642 | BRAF | 0.59314 | 7643 | STAT6 | 0.59314 | 7644 | I.S. | 0.59313 |
| 7645 | EGFR | 0.5931 | 7646 | I.S. | 0.59305 | 7647 | I.S. | 0.59302 |
| 7648 | I.S. | 0.59294 | 7649 | XRCC3 | 0.59293 | 7650 | CTNNB1 | 0.59291 |
| 7651 | DNM2 | 0.59285 | 7652 | I.S. | 0.59281 | 7653 | PRKCI | 0.59272 |
| 7654 | STAT6 | 0.59261 | 7655 | I.S. | 0.59253 | 7656 | I.S. | 0.59249 |
| 7657 | I.S. | 0.59247 | 7658 | I.S. | 0.59246 | 7659 | I.S. | 0.59244 |
| 7660 | — | 0.59242 | 7661 | I.S. | 0.5924 | 7662 | I.S. | 0.5924 |
| 7663 | BRCA1 | 0.59236 | 7664 | I.S. | 0.59234 | 7665 | SETD2 | 0.59234 |
| 7666 | I.S. | 0.59233 | 7667 | I.S. | 0.59229 | 7668 | EP300 | 0.59228 |
| 7669 | I.S. | 0.59226 | 7670 | TRAF3 | 0.59225 | 7671 | GEN1 | 0.59216 |
| 7672 | MAPK1 | 0.59216 | 7673 | I.S. | 0.5921 | 7674 | I.S. | 0.5921 |
| 7675 | NTRK2 | 0.59208 | 7676 | I.S. | 0.59192 | 7677 | I.S. | 0.59186 |
| 7678 | I.S. | 0.59186 | 7679 | ABCB7 | 0.59186 | 7680 | CHD4 | 0.59184 |
| 7681 | I.S. | 0.59184 | 7682 | I.S. | 0.59181 | 7683 | MRE11 | 0.59181 |
| 7684 | KMT2B | 0.59181 | 7685 | PLCG2 | 0.59179 | 7686 | I.S. | 0.59178 |
| 7687 | I.S. | 0.59174 | 7688 | KLHL6 | 0.59171 | 7689 | I.S. | 0.59169 |
| 7690 | KAT6A | 0.59166 | 7691 | PDGFRA | 0.5916 | 7692 | I.S. | 0.59158 |
| 7693 | I.S. | 0.59157 | 7694 | I.S. | 0.59157 | 7695 | EPHA5 | 0.59154 |
| 7696 | TCIRG1 | 0.59154 | 7697 | I.S. | 0.59153 | 7698 | I.S. | 0.59151 |
| 7699 | I.S. | 0.59151 | 7700 | PLCG1 | 0.59148 | 7701 | I.S. | 0.59146 |
| 7702 | I.S. | 0.59144 | 7703 | TCF3 | 0.59136 | 7704 | MLH1 | 0.59136 |
| 7705 | I.S. | 0.59135 | 7706 | I.S. | 0.59134 | 7707 | I.S. | 0.59132 |
| 7708 | I.S. | 0.5913 | 7709 | I.S. | 0.59129 | 7710 | KMT2B | 0.59126 |
| 7711 | I.S. | 0.59119 | 7712 | I.S. | 0.59108 | 7713 | I.S. | 0.59104 |
| 7714 | PLCG2 | 0.59103 | 7715 | I.S. | 0.591 | 7716 | I.S. | 0.59097 |
| 7717 | I.S. | 0.59095 | 7718 | I.S. | 0.59092 | 7719 | I.S. | 0.59091 |
| 7720 | I.S. | 0.59089 | 7721 | DNM2 | 0.59088 | 7722 | MED12 | 0.59085 |
| 7723 | TSC1 | 0.59083 | 7724 | I.S. | 0.59074 | 7725 | XRCC2 | 0.59073 |
| 7726 | PAK3 | 0.59068 | 7727 | RICTOR | 0.59067 | 7728 | I.S. | 0.59064 |
| 7729 | CREBBP | 0.59058 | 7730 | SMARCA4 | 0.59055 | 7731 | AXL | 0.59053 |
| 7732 | AKT1 | 0.59052 | 7733 | I.S. | 0.59051 | 7734 | I.S. | 0.59048 |
| 7735 | I.S. | 0.59047 | 7736 | ANKRD26 | 0.59044 | 7737 | PIK3CA | 0.59044 |
| 7738 | I.S. | 0.59044 | 7739 | PDGFRB | 0.59043 | 7740 | I.S. | 0.59043 |
| 7741 | KDM5A | 0.5904 | 7742 | I.S. | 0.59034 | 7743 | I.S. | 0.59032 |
| 7744 | I.S. | 0.59031 | 7745 | PMS2 | 0.59029 | 7746 | SMAD2 | 0.59026 |
| 7747 | I.S. | 0.59017 | 7748 | I.S. | 0.59017 | 7749 | I.S. | 0.59015 |
| 7750 | I.S. | 0.59007 | 7751 | I.S. | 0.59002 | 7752 | I.S. | 0.58996 |
| 7753 | I.S. | 0.58996 | 7754 | TCIRG1 | 0.58994 | 7755 | PDK1 | 0.5899 |
| 7756 | I.S. | 0.58988 | 7757 | I.S. | 0.58985 | 7758 | STAT4 | 0.58985 |
| 7759 | I.S. | 0.58984 | 7760 | NTRK2 | 0.58982 | 7761 | SPEN | 0.58982 |
| 7762 | I.S. | 0.58981 | 7763 | NOTCH3 | 0.58979 | 7764 | I.S. | 0.58975 |
| 7765 | I.S. | 0.58971 | 7766 | TSHR | 0.58967 | 7767 | I.S. | 0.58966 |
| 7768 | I.S. | 0.58961 | 7769 | I.S. | 0.5896 | 7770 | NBPF20, | 0.58954 |
| | | | | | | | NBPF19, | |
| | | | | | | | NBPF10, | |
| | | | | | | | RBM8A | |
| 7771 | CHEK2 | 0.58952 | 7772 | ATG2B | 0.58949 | 7773 | I.S. | 0.58945 |
| 7774 | ERBB4 | 0.58941 | 7775 | DDX11 | 0.5894 | 7776 | I.S. | 0.58939 |
| 7777 | FGFR4 | 0.58938 | 7778 | NSD2 | 0.58937 | 7779 | I.S. | 0.58936 |
| 7780 | I.S. | 0.58931 | 7781 | I.S. | 0.58927 | 7782 | I.S. | 0.58927 |
| 7783 | POLE | 0.58925 | 7784 | I.S. | 0.58925 | 7785 | I.S. | 0.58922 |
| 7786 | I.S. | 0.58919 | 7787 | TERT | 0.58916 | 7788 | NOTCH1 | 0.58916 |
| 7789 | FLCN | 0.58916 | 7790 | FLT4 | 0.58907 | 7791 | BCL6 | 0.58904 |
| 7792 | I.S. | 0.58903 | 7793 | I.S. | 0.589 | 7794 | TBX3 | 0.58896 |
| 7795 | AKT2 | 0.58896 | 7796 | I.S. | 0.58895 | 7797 | I.S. | 0.58889 |
| 7798 | LRP1B | 0.58883 | 7799 | LYST | 0.5888 | 7800 | ASXL1 | 0.5888 |
| 7801 | I.S. | 0.58876 | 7802 | CDK12 | 0.58874 | 7803 | TSC2 | 0.58866 |
| 7804 | I.S. | 0.5886 | 7805 | IL7R | 0.5885 | 7806 | TCF3 | 0.58849 |
| 7807 | I.S. | 0.58846 | 7808 | STAT6 | 0.58839 | 7809 | I.S. | 0.58837 |
| 7810 | I.S. | 0.58836 | 7811 | H3F3A, | 0.58827 | 7812 | I.S. | 0.58827 |
| | | | | H3F3AP4 | | | | |
| 7813 | I.S. | 0.58823 | 7814 | CDKN2A | 0.58821 | 7815 | RBBP6 | 0.58821 |
| 7816 | I.S. | 0.5882 | 7817 | POLE | 0.58818 | 7818 | ERBB3 | 0.5881 |
| 7819 | SMARCA4 | 0.58807 | 7820 | CALR | 0.58806 | 7821 | TGFBR2 | 0.58802 |
| 7822 | I.S. | 0.58799 | 7823 | I.S. | 0.58792 | 7824 | TOP2A | 0.58788 |
| 7825 | I.S. | 0.58787 | 7826 | TSHR | 0.58785 | 7827 | LYN | 0.5878 |
| 7828 | I.S. | 0.58757 | 7829 | I.S. | 0.5875 | 7830 | AURKA | 0.58749 |
| 7831 | I.S. | 0.58745 | 7832 | FANCE | 0.58736 | 7833 | I.S. | 0.5873 |
| 7834 | IRF2 | 0.5873 | 7835 | I.S. | 0.5873 | 7836 | I.S. | 0.58726 |
| 7837 | I.S. | 0.58723 | 7838 | BRCA1 | 0.58722 | 7839 | I.S. | 0.58722 |
| 7840 | FLT4 | 0.58721 | 7841 | I.S. | 0.58715 | 7842 | MAP3K14 | 0.5871 |
| 7843 | PREX2 | 0.58702 | 7844 | I.S. | 0.58702 | 7845 | I.S. | 0.587 |
| 7846 | SETD2 | 0.58699 | 7847 | FLT1 | 0.58698 | 7848 | ROS1 | 0.58697 |
| 7849 | PRKAR1A | 0.58696 | 7850 | I.S. | 0.58693 | 7851 | MTOR | 0.58683 |
| 7852 | I.S. | 0.58676 | 7853 | I.S. | 0.58675 | 7854 | I.S. | 0.58674 |
| 7855 | ERBB4 | 0.58672 | 7856 | EGFR | 0.58672 | 7857 | BRCA2 | 0.58666 |
| 7858 | I.S. | 0.58662 | 7859 | JAK1 | 0.58658 | 7860 | TNFAIP3 | 0.58655 |
| 7861 | PRKCI | 0.58652 | 7862 | I.S. | 0.5865 | 7863 | I.S. | 0.58649 |
| 7864 | SNCAIP | 0.58648 | 7865 | ASXL1 | 0.58648 | 7866 | I.S. | 0.58635 |
| 7867 | I.S. | 0.58634 | 7868 | CXCR4 | 0.58626 | 7869 | RAD21 | 0.58626 |
| 7870 | I.S. | 0.58624 | 7871 | I.S. | 0.58623 | 7872 | I.S. | 0.58622 |
| 7873 | I.S. | 0.58621 | 7874 | NOTCH2 | 0.5862 | 7875 | SEC23B | 0.58619 |
| 7876 | I.S. | 0.58618 | 7877 | JAK1 | 0.58616 | 7878 | I.S. | 0.58614 |
| 7879 | IRS2 | 0.58612 | 7880 | I.S. | 0.58612 | 7881 | LRP1B | 0.58607 |
| 7882 | MDM4 | 0.58606 | 7883 | I.S. | 0.58605 | 7884 | CBLB | 0.58604 |
| 7885 | I.S. | 0.58601 | 7886 | I.S. | 0.58599 | 7887 | I.S. | 0.58598 |
| 7888 | AXL | 0.58595 | 7889 | PSTPIP1 | 0.58584 | 7890 | I.S. | 0.58572 |
| 7891 | I.S. | 0.5857 | 7892 | STAT3 | 0.58557 | 7893 | I.S. | 0.58544 |
| 7894 | I.S. | 0.58544 | 7895 | I.S. | 0.58541 | 7896 | TOP2A | 0.58539 |
| 7897 | IKBKE | 0.58539 | 7898 | IL7R | 0.58538 | 7899 | TOP1 | 0.58537 |
| 7900 | I.S. | 0.58531 | 7901 | I.S. | 0.5853 | 7902 | I.S. | 0.58523 |
| 7903 | APC | 0.58518 | 7904 | NOTCH1 | 0.58518 | 7905 | BRD4 | 0.58515 |
| 7906 | I.S. | 0.58515 | 7907 | PDGFRB | 0.58512 | 7908 | MAP2K4 | 0.58506 |
| 7909 | ATG2B | 0.58505 | 7910 | MAP2K2 | 0.58503 | 7911 | I.S. | 0.58503 |
| 7912 | CHD4 | 0.58503 | 7913 | I.S. | 0.58488 | 7914 | DOT1L | 0.58488 |
| 7915 | I.S. | 0.58487 | 7916 | CSF3R | 0.58482 | 7917 | MAGI2 | 0.58482 |
| 7918 | RAD21 | 0.58477 | 7919 | KLC1, | 0.58465 | 7920 | I.S. | 0.58461 |
| | | | | XRCC3 | | | | |
| 7921 | KIF23 | 0.58453 | 7922 | I.S. | 0.5844 | 7923 | KEL | 0.58432 |
| 7924 | I.S. | 0.5843 | 7925 | I.S. | 0.58429 | 7926 | LRP1B | 0.58427 |
| 7927 | GALNT12 | 0.58425 | 7928 | FANCA | 0.58421 | 7929 | I.S. | 0.58417 |
| 7930 | SMARCA4 | 0.58417 | 7931 | PRKN | 0.58413 | 7932 | DICER1 | 0.5841 |
| 7933 | RICTOR | 0.5841 | 7934 | SLIT2 | 0.58409 | 7935 | I.S. | 0.58408 |
| 7936 | I.S. | 0.58404 | 7937 | I.S. | 0.58403 | 7938 | PREX2 | 0.58397 |
| 7939 | I.S. | 0.58396 | 7940 | I.S. | 0.58394 | 7941 | I.S. | 0.5839 |
| 7942 | I.S. | 0.58388 | 7943 | I.S. | 0.58387 | 7944 | CHD2 | 0.58384 |
| 7945 | ACVR1B | 0.58384 | 7946 | MEFV | 0.58369 | 7947 | I.S. | 0.58369 |
| 7948 | KMT2D | 0.58367 | 7949 | RIT1 | 0.58363 | 7950 | I.S. | 0.58363 |
| 7951 | EGFR | 0.58359 | 7952 | JAK1 | 0.58358 | 7953 | I.S. | 0.58355 |
| 7954 | IKZF3 | 0.58352 | 7955 | PIK3CA | 0.58349 | 7956 | I.S. | 0.58348 |
| 7957 | MAP3K1 | 0.58346 | 7958 | HOXB13 | 0.58343 | 7959 | ALK | 0.58332 |
| 7960 | TOP2A | 0.58325 | 7961 | RAD54L | 0.58313 | 7962 | I.S. | 0.58313 |
| 7963 | I.S. | 0.58312 | 7964 | FANCI | 0.58304 | 7965 | I.S. | 0.58302 |
| 7966 | GLI2 | 0.58301 | 7967 | I.S. | 0.58298 | 7968 | I.S. | 0.58295 |
| 7969 | I.S. | 0.58295 | 7970 | I.S. | 0.58292 | 7971 | PBRM1 | 0.58286 |
| 7972 | I.S. | 0.58286 | 7973 | STAT3 | 0.58284 | 7974 | PREX2 | 0.58281 |
| 7975 | NHP2 | 0.58277 | 7976 | CHD4 | 0.58275 | 7977 | ARID1B | 0.58275 |
| 7978 | TSC2 | 0.58274 | 7979 | NBN | 0.58272 | 7980 | EGFR | 0.58271 |
| 7981 | I.S. | 0.58266 | 7982 | I.S. | 0.58265 | 7983 | I.S. | 0.58262 |
| 7984 | CD79B | 0.58262 | 7985 | CCNE1 | 0.58257 | 7986 | I.S. | 0.58256 |
| 7987 | CD274 | 0.58254 | 7988 | I.S. | 0.58251 | 7989 | I.S. | 0.58248 |
| 7990 | PIK3CB | 0.58241 | 7991 | KMT2D | 0.5824 | 7992 | BRIP1 | 0.58237 |
| 7993 | TSC1 | 0.58231 | 7994 | LRP1B | 0.58229 | 7995 | CTCF | 0.58229 |
| 7996 | FLT3 | 0.58227 | 7997 | EP300 | 0.58225 | 7998 | I.S. | 0.58218 |
| 7999 | MAP2K1 | 0.58208 | 8000 | I.S. | 0.58205 | 8001 | FANCG | 0.58182 |
| 8002 | KMT2B | 0.58177 | 8003 | I.S. | 0.58175 | 8004 | I.S. | 0.58168 |
| 8005 | I.S. | 0.58162 | 8006 | LRP1B | 0.58161 | 8007 | I.S. | 0.58161 |
| 8008 | NTRK3 | 0.58152 | 8009 | I.S. | 0.58152 | 8010 | I.S. | 0.5815 |
| 8011 | I.S. | 0.58146 | 8012 | RAF1 | 0.58143 | 8013 | CEBPA | 0.58141 |
| 8014 | I.S. | 0.58139 | 8015 | I.S. | 0.58136 | 8016 | I.S. | 0.58134 |
| 8017 | KMT2A | 0.58132 | 8018 | I.S. | 0.58131 | 8019 | FANCM | 0.58129 |
| 8020 | CTNNA1 | 0.58126 | 8021 | I.S. | 0.5812 | 8022 | I.S. | 0.5812 |
| 8023 | I.S. | 0.58117 | 8024 | NLRP3 | 0.58111 | 8025 | I.S. | 0.58105 |
| 8026 | RB1 | 0.58102 | 8027 | I.S. | 0.58092 | 8028 | I.S. | 0.58092 |
| 8029 | I.S. | 0.58092 | 8030 | I.S. | 0.58088 | 8031 | ANKRD26 | 0.58088 |
| 8032 | ATG2B | 0.58087 | 8033 | GLI1 | 0.58087 | 8034 | I.S. | 0.58084 |
| 8035 | I.S. | 0.58074 | 8036 | I.S. | 0.58071 | 8037 | I.S. | 0.5807 |
| 8038 | AKT1 | 0.58067 | 8039 | CDK4 | 0.58066 | 8040 | FANCD2 | 0.58064 |
| 8041 | I.S. | 0.58064 | 8042 | BLM | 0.58058 | 8043 | ARFRP1 | 0.58053 |
| 8044 | I.S. | 0.58052 | 8045 | BRCA1 | 0.58046 | 8046 | CXCR4 | 0.58043 |
| 8047 | FLT4 | 0.58043 | 8048 | LRP1B | 0.5804 | 8049 | I.S. | 0.58039 |
| 8050 | DNM2 | 0.58038 | 8051 | GRM3 | 0.58027 | 8052 | RARA | 0.58023 |
| 8053 | I.S. | 0.58023 | 8054 | I.S. | 0.58023 | 8055 | TCF3 | 0.58022 |
| 8056 | I.S. | 0.58022 | 8057 | PRSS1 | 0.58021 | 8058 | TSC2 | 0.58019 |
| 8059 | I.S. | 0.58007 | 8060 | RUNX1 | 0.58001 | 8061 | I.S. | 0.57988 |
| 8062 | BCL2L2 | 0.57984 | 8063 | I.S. | 0.57969 | 8064 | ARID2 | 0.57963 |
| 8065 | I.S. | 0.57962 | 8066 | FANCM | 0.5796 | 8067 | MET | 0.5796 |
| 8068 | SDHD | 0.57957 | 8069 | I.S. | 0.5795 | 8070 | I.S. | 0.57947 |
| 8071 | BRIP1 | 0.57937 | 8072 | I.S. | 0.57924 | 8073 | ALK | 0.57915 |
| 8074 | I.S. | 0.57915 | 8075 | I.S. | 0.57904 | 8076 | MEN1 | 0.579 |
| 8077 | NTRK3 | 0.57897 | 8078 | I.S. | 0.57896 | 8079 | I.S. | 0.57894 |
| 8080 | I.S. | 0.5789 | 8081 | I.S. | 0.5788 | 8082 | NOTCH1 | 0.57878 |
| 8083 | DICER1 | 0.57871 | 8084 | CDH1 | 0.57864 | 8085 | CUL3 | 0.57862 |
| 8086 | CCN6 | 0.57861 | 8087 | I.S. | 0.57859 | 8088 | RB1 | 0.57859 |
| 8089 | I.S. | 0.57855 | 8090 | I.S. | 0.57853 | 8091 | NSD1 | 0.57853 |
| 8092 | I.S. | 0.57845 | 8093 | I.S. | 0.57841 | 8094 | MRE11 | 0.57837 |
| 8095 | PRKCI | 0.57831 | 8096 | I.S. | 0.57828 | 8097 | I.S. | 0.57821 |
| 8098 | RICTOR | 0.5782 | 8099 | I.S. | 0.5782 | 8100 | BRCA1 | 0.57816 |
| 8101 | I.S. | 0.57815 | 8102 | I.S. | 0.57814 | 8103 | SMARCA4 | 0.57811 |
| 8104 | I.S. | 0.57811 | 8105 | PTCH1 | 0.57808 | 8106 | I.S. | 0.57801 |
| 8107 | I.S. | 0.57794 | 8108 | NF1 | 0.57789 | 8109 | I.S. | 0.57785 |
| 8110 | RARA | 0.57784 | 8111 | I.S. | 0.57783 | 8112 | I.S. | 0.57781 |
| 8113 | CREBBP | 0.57772 | 8114 | PRSS1 | 0.57769 | 8115 | I.S. | 0.57763 |
| 8116 | NPM1 | 0.57759 | 8117 | I.S. | 0.57749 | 8118 | I.S. | 0.57749 |
| 8119 | FGFR4 | 0.57743 | 8120 | I.S. | 0.57731 | 8121 | I.S. | 0.5773 |
| 8122 | I.S. | 0.57724 | 8123 | I.S. | 0.57724 | 8124 | I.S. | 0.57724 |
| 8125 | I.S. | 0.5772 | 8126 | DDX11 | 0.57716 | 8127 | I.S. | 0.57709 |
| 8128 | I.S. | 0.57709 | 8129 | ADGRA2 | 0.57706 | 8130 | I.S. | 0.57701 |
| 8131 | I.S. | 0.57693 | 8132 | I.S. | 0.57692 | 8133 | NBN | 0.57691 |
| 8134 | SMAD2 | 0.57687 | 8135 | I.S. | 0.57685 | 8136 | I.S. | 0.57682 |
| 8137 | I.S. | 0.57671 | 8138 | I.S. | 0.57668 | 8139 | PRKCI | 0.57668 |
| 8140 | PRKCI | 0.57648 | 8141 | I.S. | 0.57648 | 8142 | MYCNOS, | 0.57647 |
| | | | | | | | MYCN, MYCN | |
| 8143 | LRP1B | 0.57636 | 8144 | I.S. | 0.57636 | 8145 | HAX1 | 0.57635 |
| 8146 | I.S. | 0.57633 | 8147 | I.S. | 0.57631 | 8148 | EP300 | 0.57629 |
| 8149 | CD79A | 0.57627 | 8150 | I.S. | 0.57621 | 8151 | JAK3 | 0.57618 |
| 8152 | I.S. | 0.57616 | 8153 | IL7R | 0.57615 | 8154 | I.S. | 0.57603 |
| 8155 | CDH1 | 0.57603 | 8156 | I.S. | 0.57602 | 8157 | AKT1 | 0.57602 |
| 8158 | CDK12 | 0.57581 | 8159 | I.S. | 0.5758 | 8160 | SDHD | 0.57577 |
| 8161 | PDGFRB | 0.57574 | 8162 | KMT2B | 0.57567 | 8163 | I.S. | 0.57564 |
| 8164 | AXIN1 | 0.57562 | 8165 | I.S. | 0.57549 | 8166 | ERBB2 | 0.57547 |
| 8167 | I.S. | 0.57543 | 8168 | I.S. | 0.57541 | 8169 | I.S. | 0.57541 |
| 8170 | I.S. | 0.57538 | 8171 | EZH2 | 0.57537 | 8172 | I.S. | 0.57536 |
| 8173 | I.S. | 0.57531 | 8174 | ARID1A | 0.57522 | 8175 | I.S. | 0.57521 |
| 8176 | MEF2B | 0.57514 | 8177 | KMT2A | 0.57513 | 8178 | NROB1 | 0.57506 |
| 8179 | I.S. | 0.57504 | 8180 | I.S. | 0.57503 | 8181 | I.S. | 0.57499 |
| 8182 | LRP1B | 0.57498 | 8183 | GNAS | 0.57496 | 8184 | KIF23 | 0.5749 |
| 8185 | I.S. | 0.57483 | 8186 | MYCL | 0.57479 | 8187 | CARD11 | 0.57475 |
| 8188 | I.S. | 0.57474 | 8189 | MYCL | 0.57474 | 8190 | BRIP1 | 0.57473 |
| 8191 | I.S. | 0.5747 | 8192 | EP300 | 0.57464 | 8193 | INPP4B | 0.57464 |
| 8194 | I.S. | 0.57453 | 8195 | INPP4B | 0.57452 | 8196 | I.S. | 0.57452 |
| 8197 | MTOR | 0.57451 | 8198 | I.S. | 0.57443 | 8199 | NOTCH3 | 0.5744 |
| 8200 | ARID1B | 0.57434 | 8201 | ARID2 | 0.57432 | 8202 | HNF1A | 0.57431 |
| 8203 | MAP2K2 | 0.57424 | 8204 | I.S. | 0.57421 | 8205 | I.S. | 0.57419 |
| 8206 | FANCA | 0.5741 | 8207 | CHD2 | 0.57405 | 8208 | FAS | 0.57401 |
| 8209 | NTRK3 | 0.57389 | 8210 | I.S. | 0.57388 | 8211 | MAP3K14 | 0.57386 |
| 8212 | PLCG2 | 0.57386 | 8213 | I.S. | 0.57385 | 8214 | I.S. | 0.57384 |
| 8215 | I.S. | 0.57384 | 8216 | ARID2 | 0.5738 | 8217 | CARD11 | 0.5738 |
| 8218 | BLM | 0.5738 | 8219 | I.S. | 0.57378 | 8220 | I.S. | 0.57376 |
| 8221 | MSH6 | 0.57375 | 8222 | I.S. | 0.57371 | 8223 | I.S. | 0.57369 |
| 8224 | I.S. | 0.57365 | 8225 | ATR | 0.57354 | 8226 | I.S. | 0.57352 |
| 8227 | I.S. | 0.57348 | 8228 | FANCD2 | 0.57341 | 8229 | CTNNA1 | 0.57336 |
| 8230 | FANCB | 0.57333 | 8231 | PIK3R1 | 0.5733 | 8232 | KMT2C | 0.57326 |
| 8233 | ROS1 | 0.57323 | 8234 | I.S. | 0.57321 | 8235 | PALB2 | 0.57318 |
| 8236 | MEFV | 0.57311 | 8237 | MUTYH | 0.57309 | 8238 | I.S. | 0.57307 |
| 8239 | I.S. | 0.57307 | 8240 | DOT1L | 0.57294 | 8241 | I.S. | 0.57293 |
| 8242 | I.S. | 0.57285 | 8243 | I.S. | 0.57285 | 8244 | I.S. | 0.57278 |
| 8245 | APC | 0.57271 | 8246 | SLX4 | 0.5727 | 8247 | PBRM1 | 0.57262 |
| 8248 | I.S. | 0.57261 | 8249 | KMT2D | 0.57256 | 8250 | I.S. | 0.57243 |
| 8251 | I.S. | 0.57229 | 8252 | I.S. | 0.57223 | 8253 | SMARCA4 | 0.57222 |
| 8254 | I.S. | 0.57221 | 8255 | I.S. | 0.57221 | 8256 | I.S. | 0.57219 |
| 8257 | NOTCH1 | 0.57214 | 8258 | I.S. | 0.57208 | 8259 | DICER1 | 0.57208 |
| 8260 | FANCM | 0.57208 | 8261 | I.S. | 0.57202 | 8262 | AURKA | 0.57191 |
| 8263 | FGFR1 | 0.5719 | 8264 | I.S. | 0.57183 | 8265 | MEN1 | 0.5717 |
| 8266 | PPM1D | 0.57167 | 8267 | I.S. | 0.57166 | 8268 | I.S. | 0.57163 |
| 8269 | RB1 | 0.57161 | 8270 | I.S. | 0.5716 | 8271 | I.S. | 0.57155 |
| 8272 | I.S. | 0.57145 | 8273 | I.S. | 0.57145 | 8274 | I.S. | 0.57142 |
| 8275 | I.S. | 0.5714 | 8276 | I.S. | 0.57138 | 8277 | I.S. | 0.57137 |
| 8278 | CDAN1 | 0.57137 | 8279 | CDK12 | 0.57134 | 8280 | RBBP6 | 0.57131 |
| 8281 | I.S. | 0.57124 | 8282 | ATG2B | 0.57119 | 8283 | NLRP3 | 0.57114 |
| 8284 | SLX4 | 0.57113 | 8285 | I.S. | 0.57113 | 8286 | I.S. | 0.57112 |
| 8287 | I.S. | 0.5711 | 8288 | I.S. | 0.57105 | 8289 | I.S. | 0.57101 |
| 8290 | I.S. | 0.57092 | 8291 | I.S. | 0.57091 | 8292 | CSF3R | 0.57089 |
| 8293 | PRKAR1A | 0.57083 | 8294 | I.S. | 0.57082 | 8295 | GALNT12 | 0.57077 |
| 8296 | ATG2B | 0.57077 | 8297 | I.S. | 0.57071 | 8298 | I.S. | 0.57069 |
| 8299 | NUP93 | 0.57068 | 8300 | JAK1 | 0.5706 | 8301 | I.S. | 0.57057 |
| 8302 | I.S. | 0.57049 | 8303 | RTEL1 | 0.57048 | 8304 | I.S. | 0.57047 |
| 8305 | I.S. | 0.57047 | 8306 | I.S. | 0.57045 | 8307 | I.S. | 0.57042 |
| 8308 | TGFBR2 | 0.57035 | 8309 | CDK8 | 0.57034 | 8310 | I.S. | 0.57032 |
| 8311 | LRP1B | 0.57032 | 8312 | RUNX1T1 | 0.5703 | 8313 | I.S. | 0.5703 |
| 8314 | I.S. | 0.57029 | 8315 | I.S. | 0.57024 | 8316 | SBDS | 0.57021 |
| 8317 | I.S. | 0.5702 | 8318 | I.S. | 0.57011 | 8319 | ATR | 0.57009 |
| 8320 | I.S. | 0.57008 | 8321 | ABL1 | 0.57008 | 8322 | SMO | 0.57006 |
| 8323 | AR | 0.57003 | 8324 | PMS2 | 0.57003 | 8325 | I.S. | 0.57 |
| 8326 | SLIT2 | 0.56999 | 8327 | I.S. | 0.56989 | 8328 | I.S. | 0.56985 |
| 8329 | DOT1L | 0.56982 | 8330 | I.S. | 0.56974 | 8331 | IKZF3 | 0.56967 |
| 8332 | I.S. | 0.56957 | 8333 | I.S. | 0.56953 | 8334 | I.S. | 0.56949 |
| 8335 | CARD11 | 0.56938 | 8336 | SPTA1 | 0.56935 | 8337 | GLI2 | 0.56931 |
| 8338 | I.S. | 0.56929 | 8339 | I.S. | 0.56923 | 8340 | I.S. | 0.56903 |
| 8341 | KMT2D | 0.56902 | 8342 | RBBP6 | 0.56902 | 8343 | PTCH1 | 0.569 |
| 8344 | I.S. | 0.56897 | 8345 | I.S. | 0.56896 | 8346 | I.S. | 0.56896 |
| 8347 | I.S. | 0.56894 | 8348 | I.S. | 0.56891 | 8349 | I.S. | 0.56885 |
| 8350 | I.S. | 0.56879 | 8351 | I.S. | 0.56875 | 8352 | I.S. | 0.56874 |
| 8353 | JAK1 | 0.5687 | 8354 | I.S. | 0.56866 | 8355 | I.S. | 0.56866 |
| 8356 | I.S. | 0.56859 | 8357 | I.S. | 0.56858 | 8358 | RTEL1 | 0.56858 |
| 8359 | KMT2D | 0.56852 | 8360 | LRP1B | 0.56843 | 8361 | I.S. | 0.56837 |
| 8362 | TCF3 | 0.56837 | 8363 | RPTOR | 0.56836 | 8364 | RPTOR | 0.56828 |
| 8365 | I.S. | 0.56826 | 8366 | WT1 | 0.56813 | 8367 | I.S. | 0.568 |
| 8368 | I.S. | 0.56791 | 8369 | I.S. | 0.56789 | 8370 | I.S. | 0.56787 |
| 8371 | I.S. | 0.56784 | 8372 | I.S. | 0.56783 | 8373 | I.S. | 0.5678 |
| 8374 | ERBB3 | 0.56772 | 8375 | MTOR | 0.56765 | 8376 | I.S. | 0.56764 |
| 8377 | I.S. | 0.56759 | 8378 | I.S. | 0.56757 | 8379 | I.S. | 0.56751 |
| 8380 | I.S. | 0.5675 | 8381 | I.S. | 0.56735 | 8382 | I.S. | 0.56735 |
| 8383 | I.S. | 0.56729 | 8384 | VHL | 0.56718 | 8385 | I.S. | 0.56715 |
| 8386 | PLCG2 | 0.56713 | 8387 | I.S. | 0.56703 | 8388 | I.S. | 0.56694 |
| 8389 | MAP2K2 | 0.56691 | 8390 | PBRM1 | 0.56688 | 8391 | RAD54L | 0.56683 |
| 8392 | I.S. | 0.56677 | 8393 | I.S. | 0.56673 | 8394 | CARD11 | 0.56671 |
| 8395 | I.S. | 0.5667 | 8396 | GSK3B | 0.56669 | 8397 | PIK3R2 | 0.56659 |
| 8398 | I.S. | 0.56657 | 8399 | I.S. | 0.56652 | 8400 | I.S. | 0.5665 |
| 8401 | BARD1 | 0.56647 | 8402 | I.S. | 0.56647 | 8403 | ATM | 0.56647 |
| 8404 | I.S. | 0.56645 | 8405 | RPTOR | 0.56643 | 8406 | I.S. | 0.56641 |
| 8407 | I.S. | 0.56641 | 8408 | I.S. | 0.5663 | 8409 | I.S. | 0.56628 |
| 8410 | I.S. | 0.56623 | 8411 | I.S. | 0.56622 | 8412 | POLE | 0.5662 |
| 8413 | I.S. | 0.56618 | 8414 | I.S. | 0.56617 | 8415 | I.S. | 0.56606 |
| 8416 | NOTCH1 | 0.56602 | 8417 | I.S. | 0.56601 | 8418 | TOP1 | 0.56599 |
| 8419 | I.S. | 0.56595 | 8420 | CDAN1 | 0.56585 | 8421 | PREX2 | 0.56584 |
| 8422 | AK2 | 0.56578 | 8423 | BCORL1 | 0.56568 | 8424 | I.S. | 0.56557 |
| 8425 | TSC2 | 0.56554 | 8426 | RAD51C | 0.56548 | 8427 | GATA4 | 0.56543 |
| 8428 | RUNX1T1 | 0.56537 | 8429 | I.S. | 0.56533 | 8430 | MVK | 0.56526 |
| 8431 | HGF | 0.56504 | 8432 | NFKBIA | 0.56501 | 8433 | SPTA1 | 0.56498 |
| 8434 | RB1 | 0.56485 | 8435 | I.S. | 0.56481 | 8436 | RAB27A | 0.56479 |
| 8437 | I.S. | 0.56478 | 8438 | PRKAR1A | 0.56471 | 8439 | I.S. | 0.56468 |
| 8440 | I.S. | 0.56456 | 8441 | I.S. | 0.56456 | 8442 | LRP1B | 0.56455 |
| 8443 | RPTOR | 0.56453 | 8444 | I.S. | 0.56445 | 8445 | ATR | 0.56442 |
| 8446 | I.S. | 0.56439 | 8447 | I.S. | 0.56438 | 8448 | KLF1 | 0.56433 |
| 8449 | NOTCH1 | 0.5643 | 8450 | SETD2 | 0.56427 | 8451 | EPHA5 | 0.56424 |
| 8452 | I.S. | 0.56418 | 8453 | KEAP1 | 0.56417 | 8454 | PMS2 | 0.56412 |
| 8455 | I.S. | 0.5641 | 8456 | I.S. | 0.56402 | 8457 | POLE | 0.56394 |
| 8458 | MEN1 | 0.56385 | 8459 | RET | 0.56384 | 8460 | I.S. | 0.56379 |
| 8461 | I.S. | 0.56376 | 8462 | PRKDC | 0.56373 | 8463 | PIK3C2B | 0.56367 |
| 8464 | I.S. | 0.56364 | 8465 | MAP2K1, | 0.5636 | 8466 | I.S. | 0.56352 |
| | | | | SNAPC5 | | | | |
| 8467 | CALR, | 0.56347 | 8468 | SMARCA4 | 0.56335 | 8469 | BRCA2 | 0.56328 |
| | MIR6515 | | | | | | | |
| 8470 | I.S. | 0.56324 | 8471 | ATR | 0.56311 | 8472 | I.S. | 0.56307 |
| 8473 | CBLB | 0.56303 | 8474 | SPTA1 | 0.56301 | 8475 | I.S. | 0.56299 |
| 8476 | RAD51B | 0.56297 | 8477 | NF1 | 0.56295 | 8478 | I.S. | 0.56293 |
| 8479 | LPIN2 | 0.56284 | 8480 | I.S. | 0.56278 | 8481 | MDM2 | 0.56278 |
| 8482 | CTCF | 0.56274 | 8483 | I.S. | 0.56266 | 8484 | I.S. | 0.56261 |
| 8485 | MPL | 0.5626 | 8486 | KMT2C | 0.56258 | 8487 | I.S. | 0.56257 |
| 8488 | NSD1 | 0.56256 | 8489 | I.S. | 0.56252 | 8490 | SLX4 | 0.56246 |
| 8491 | STAT4 | 0.56245 | 8492 | I.S. | 0.56245 | 8493 | I.S. | 0.56244 |
| 8494 | MAP2K2 | 0.56237 | 8495 | CCND3 | 0.56232 | 8496 | I.S. | 0.56218 |
| 8497 | PLCG1 | 0.56216 | 8498 | I.S. | 0.56215 | 8499 | ATR | 0.56212 |
| 8500 | I.S. | 0.56211 | 8501 | LRP1B | 0.56207 | 8502 | PIK3CA | 0.56203 |
| 8503 | KLF1 | 0.56198 | 8504 | ASXL1 | 0.56197 | 8505 | KMT2B | 0.5619 |
| 8506 | FANCD2 | 0.56184 | 8507 | I.S. | 0.56183 | 8508 | RAD51B | 0.56182 |
| 8509 | I.S. | 0.56176 | 8510 | I.S. | 0.56174 | 8511 | STAT4 | 0.56174 |
| 8512 | APC | 0.56171 | 8513 | TERT | 0.56161 | 8514 | I.S. | 0.56154 |
| 8515 | AK2 | 0.56153 | 8516 | KAT6A | 0.56151 | 8517 | I.S. | 0.56148 |
| 8518 | AURKB | 0.56144 | 8519 | BLM | 0.56139 | 8520 | I.S. | 0.56133 |
| 8521 | ASXL1 | 0.56133 | 8522 | SLIT2 | 0.56121 | 8523 | ERBB4 | 0.5612 |
| 8524 | KMT2C | 0.5611 | 8525 | I.S. | 0.56105 | 8526 | I.S. | 0.56103 |
| 8527 | PREX2 | 0.56097 | 8528 | BRD4 | 0.56091 | 8529 | IGF1R | 0.56088 |
| 8530 | KMT2D | 0.56085 | 8531 | PLCG2 | 0.56084 | 8532 | I.S. | 0.56083 |
| 8533 | I.S. | 0.56078 | 8534 | NF1 | 0.56074 | 8535 | I.S. | 0.5607 |
| 8536 | I.S. | 0.56066 | 8537 | KMT2B | 0.56059 | 8538 | I.S. | 0.56058 |
| 8539 | SOX9 | 0.56053 | 8540 | I.S. | 0.56052 | 8541 | SPTA1 | 0.5605 |
| 8542 | SEC23B | 0.5605 | 8543 | I.S. | 0.56047 | 8544 | GEN1 | 0.56047 |
| 8545 | I.S. | 0.56044 | 8546 | STAT6 | 0.56043 | 8547 | I.S. | 0.56043 |
| 8548 | I.S. | 0.56037 | 8549 | KDR | 0.56029 | 8550 | I.S. | 0.56029 |
| 8551 | I.S. | 0.56029 | 8552 | SPTA1 | 0.56028 | 8553 | I.S. | 0.56017 |
| 8554 | AR | 0.56009 | 8555 | I.S. | 0.56008 | 8556 | I.S. | 0.56007 |
| 8557 | I.S. | 0.56003 | 8558 | BRIP1 | 0.55999 | 8559 | I.S. | 0.55985 |
| 8560 | I.S. | 0.55984 | 8561 | I.S. | 0.55983 | 8562 | I.S. | 0.55981 |
| 8563 | I.S. | 0.55981 | 8564 | I.S. | 0.55979 | 8565 | CDC73 | 0.55979 |
| 8566 | SPEN | 0.55978 | 8567 | BTK | 0.55976 | 8568 | ACVR1B | 0.55973 |
| 8569 | I.S. | 0.5597 | 8570 | I.S. | 0.55968 | 8571 | I.S. | 0.55968 |
| 8572 | I.S. | 0.55967 | 8573 | GREM1 | 0.55963 | 8574 | I.S. | 0.55953 |
| 8575 | AMER1 | 0.5595 | 8576 | ERBB3 | 0.55949 | 8577 | PIK3CB | 0.55948 |
| 8578 | I.S. | 0.55942 | 8579 | HOXA11 | 0.55942 | 8580 | SF3B1 | 0.5594 |
| 8581 | I.S. | 0.55937 | 8582 | I.S. | 0.55936 | 8583 | I.S. | 0.55935 |
| 8584 | I.S. | 0.55932 | 8585 | I.S. | 0.5593 | 8586 | CREBBP | 0.55924 |
| 8587 | I.S. | 0.55921 | 8588 | I.S. | 0.5592 | 8589 | ATM | 0.55919 |
| 8590 | ATG2B | 0.55912 | 8591 | I.S. | 0.55903 | 8592 | MSH6 | 0.55898 |
| 8593 | I.S. | 0.55891 | 8594 | MDM4 | 0.55889 | 8595 | I.S. | 0.55884 |
| 8596 | I.S. | 0.55863 | 8597 | CUL3 | 0.55862 | 8598 | I.S. | 0.5586 |
| 8599 | I.S. | 0.55859 | 8600 | I.S. | 0.55855 | 8601 | KMT2A | 0.55851 |
| 8602 | RUNX1T1 | 0.55848 | 8603 | I.S. | 0.55835 | 8604 | BRCA2 | 0.55833 |
| 8605 | MTOR | 0.55829 | 8606 | AKT1 | 0.55824 | 8607 | MITF | 0.5582 |
| 8608 | I.S. | 0.55813 | 8609 | ADA | 0.55813 | 8610 | FANCD2 | 0.55806 |
| 8611 | SETD2 | 0.558 | 8612 | I.S. | 0.558 | 8613 | I.S. | 0.55797 |
| 8614 | I.S. | 0.55796 | 8615 | SPTA1 | 0.5579 | 8616 | FOXP1 | 0.55788 |
| 8617 | RTEL1 | 0.55788 | 8618 | I.S. | 0.55779 | 8619 | I.S. | 0.55779 |
| 8620 | I.S. | 0.5577 | 8621 | I.S. | 0.55764 | 8622 | I.S. | 0.55758 |
| 8623 | I.S. | 0.55755 | 8624 | I.S. | 0.55749 | 8625 | I.S. | 0.55746 |
| 8626 | I.S. | 0.5574 | 8627 | SAMD9L | 0.55738 | 8628 | I.S. | 0.55737 |
| 8629 | I.S. | 0.55726 | 8630 | I.S. | 0.55722 | 8631 | STAT4 | 0.55722 |
| 8632 | AXL | 0.55717 | 8633 | SETD2 | 0.55713 | 8634 | I.S. | 0.5571 |
| 8635 | BCR | 0.55707 | 8636 | CHEK2 | 0.55703 | 8637 | I.S. | 0.55702 |
| 8638 | GNAQ | 0.55702 | 8639 | I.S. | 0.55701 | 8640 | I.S. | 0.55684 |
| 8641 | FAS | 0.55684 | 8642 | I.S. | 0.55678 | 8643 | I.S. | 0.55672 |
| 8644 | I.S. | 0.55669 | 8645 | POT1 | 0.55656 | 8646 | I.S. | 0.55653 |
| 8647 | I.S. | 0.55653 | 8648 | I.S. | 0.55651 | 8649 | I.S. | 0.55649 |
| 8650 | I.S. | 0.55644 | 8651 | I.S. | 0.55642 | 8652 | I.S. | 0.55632 |
| 8653 | I.S. | 0.5563 | 8654 | ATR | 0.55627 | 8655 | I.S. | 0.55625 |
| 8656 | TSHR | 0.55622 | 8657 | BRCA1 | 0.55613 | 8658 | I.S. | 0.55612 |
| 8659 | I.S. | 0.55607 | 8660 | CDK6 | 0.55606 | 8661 | I.S. | 0.55602 |
| 8662 | I.S. | 0.55601 | 8663 | I.S. | 0.55592 | 8664 | LRP1B | 0.55591 |
| 8665 | I.S. | 0.55589 | 8666 | — | 0.5558 | 8667 | I.S. | 0.55566 |
| 8668 | I.S. | 0.55563 | 8669 | I.S. | 0.55553 | 8670 | FANCI | 0.55545 |
| 8671 | I.S. | 0.55545 | 8672 | I.S. | 0.55539 | 8673 | FANCM | 0.55538 |
| 8674 | EP300 | 0.55536 | 8675 | DNM2 | 0.55536 | 8676 | TOP2A | 0.55533 |
| 8677 | SPTA1 | 0.55532 | 8678 | AMER1 | 0.55531 | 8679 | KMT2C | 0.55527 |
| 8680 | I.S. | 0.5552 | 8681 | I.S. | 0.5552 | 8682 | ASXL1 | 0.55518 |
| 8683 | KEAP1 | 0.55512 | 8684 | LYST | 0.55509 | 8685 | ATR | 0.55494 |
| 8686 | I.S. | 0.55491 | 8687 | I.S. | 0.55491 | 8688 | AXL | 0.55485 |
| 8689 | I.S. | 0.55485 | 8690 | I.S. | 0.55481 | 8691 | I.S. | 0.55478 |
| 8692 | KEL | 0.55477 | 8693 | I.S. | 0.55477 | 8694 | I.S. | 0.55476 |
| 8695 | I.S. | 0.55473 | 8696 | I.S. | 0.55473 | 8697 | I.S. | 0.55472 |
| 8698 | SLX4 | 0.55471 | 8699 | CDKN2A | 0.5547 | 8700 | RUNX1 | 0.55468 |
| 8701 | ARID1A | 0.55463 | 8702 | I.S. | 0.55449 | 8703 | I.S. | 0.55447 |
| 8704 | RANBP2 | 0.55446 | 8705 | I.S. | 0.55445 | 8706 | ATG2B | 0.55443 |
| 8707 | I.S. | 0.55441 | 8708 | I.S. | 0.55426 | 8709 | MET | 0.55419 |
| 8710 | LPIN2 | 0.55417 | 8711 | I.S. | 0.55416 | 8712 | KDR | 0.55415 |
| 8713 | I.S. | 0.55409 | 8714 | SLX4 | 0.55407 | 8715 | EED | 0.55406 |
| 8716 | I.S. | 0.55405 | 8717 | IRS2 | 0.55401 | 8718 | EPHB1 | 0.554 |
| 8719 | SMARCA4 | 0.55399 | 8720 | KMT2A | 0.55396 | 8721 | I.S. | 0.55392 |
| 8722 | LRP1B | 0.5539 | 8723 | MEFV | 0.5539 | 8724 | I.S. | 0.5537 |
| 8725 | TSC2 | 0.55369 | 8726 | ANKRD26 | 0.55365 | 8727 | CBLB | 0.5536 |
| 8728 | I.S. | 0.55352 | 8729 | MDM2 | 0.55351 | 8730 | ADGRA2 | 0.55348 |
| 8731 | I.S. | 0.55346 | 8732 | PTPN11 | 0.55344 | 8733 | ERBB3 | 0.55341 |
| 8734 | I.S. | 0.55337 | 8735 | BCR | 0.55336 | 8736 | I.S. | 0.55333 |
| 8737 | I.S. | 0.55327 | 8738 | I.S. | 0.55324 | 8739 | KMT2C | 0.55321 |
| 8740 | I.S. | 0.55319 | 8741 | JAK1 | 0.55313 | 8742 | I.S. | 0.55311 |
| 8743 | I.S. | 0.55305 | 8744 | PMS1 | 0.55304 | 8745 | PREX2 | 0.55301 |
| 8746 | I.S. | 0.55295 | 8747 | I.S. | 0.55294 | 8748 | I.S. | 0.55292 |
| 8749 | CDAN1 | 0.55292 | 8750 | I.S. | 0.55287 | 8751 | RANBP2 | 0.55287 |
| 8752 | SLX4 | 0.55286 | 8753 | SPEN | 0.55282 | 8754 | I.S. | 0.5528 |
| 8755 | POLE | 0.55271 | 8756 | I.S. | 0.55271 | 8757 | JAK1 | 0.55269 |
| 8758 | I.S. | 0.55254 | 8759 | I.S. | 0.55252 | 8760 | NOTCH1 | 0.55247 |
| 8761 | I.S. | 0.55247 | 8762 | I.S. | 0.55247 | 8763 | I.S. | 0.55245 |
| 8764 | I.S. | 0.55244 | 8765 | I.S. | 0.55242 | 8766 | I.S. | 0.55236 |
| 8767 | KEL | 0.5523 | 8768 | CDKN2B | 0.5523 | 8769 | I.S. | 0.55218 |
| 8770 | I.S. | 0.55217 | 8771 | SEC23B | 0.55215 | 8772 | I.S. | 0.55208 |
| 8773 | I.S. | 0.55206 | 8774 | FAT1 | 0.55204 | 8775 | I.S. | 0.55195 |
| 8776 | I.S. | 0.55187 | 8777 | KDR | 0.55185 | 8778 | STK11 | 0.55184 |
| 8779 | KMT2C | 0.55183 | 8780 | PLCG1 | 0.55182 | 8781 | I.S. | 0.55173 |
| 8782 | I.S. | 0.55171 | 8783 | I.S. | 0.5517 | 8784 | SPOP | 0.55159 |
| 8785 | RUNX1 | 0.55158 | 8786 | I.S. | 0.55155 | 8787 | MUTYH | 0.55155 |
| 8788 | BARD1 | 0.55152 | 8789 | SPEN | 0.55143 | 8790 | I.S. | 0.5514 |
| 8791 | I.S. | 0.55138 | 8792 | MDM4 | 0.55138 | 8793 | ZRSR2 | 0.55134 |
| 8794 | I.S. | 0.55134 | 8795 | ABCB7 | 0.55129 | 8796 | PLCG1 | 0.55128 |
| 8797 | I.S. | 0.55126 | 8798 | SF3B1 | 0.55117 | 8799 | I.S. | 0.55112 |
| 8800 | I.S. | 0.5511 | 8801 | KIT | 0.55106 | 8802 | KMT2C | 0.55102 |
| 8803 | I.S. | 0.55098 | 8804 | I.S. | 0.55095 | 8805 | I.S. | 0.55093 |
| 8806 | AXIN1 | 0.55087 | 8807 | I.S. | 0.55075 | 8808 | RBM10 | 0.55073 |
| 8809 | ABL2 | 0.55073 | 8810 | I.S. | 0.55073 | 8811 | I.S. | 0.55067 |
| 8812 | I.S. | 0.55057 | 8813 | I.S. | 0.55055 | 8814 | NOTCH1 | 0.55048 |
| 8815 | I.S. | 0.55034 | 8816 | KMT2B | 0.55033 | 8817 | I.S. | 0.55031 |
| 8818 | I.S. | 0.55027 | 8819 | EP300 | 0.55024 | 8820 | CARD11 | 0.55021 |
| 8821 | I.S. | 0.55019 | 8822 | I.S. | 0.55018 | 8823 | BRAF | 0.5501 |
| 8824 | LYST | 0.55005 | 8825 | I.S. | 0.55004 | 8826 | TRAF3 | 0.54995 |
| 8827 | NF1 | 0.54987 | 8828 | I.S. | 0.54987 | 8829 | I.S. | 0.54986 |
| 8830 | NBN | 0.54982 | 8831 | I.S. | 0.54982 | 8832 | I.S. | 0.54977 |
| 8833 | NUP93 | 0.54977 | 8834 | I.S. | 0.54972 | 8835 | I.S. | 0.54971 |
| 8836 | I.S. | 0.54967 | 8837 | I.S. | 0.54963 | 8838 | CUX1 | 0.5496 |
| 8839 | I.S. | 0.54959 | 8840 | TERT | 0.54953 | 8841 | DOT1L | 0.54948 |
| 8842 | I.S. | 0.54947 | 8843 | GNA13 | 0.54945 | 8844 | I.S. | 0.54938 |
| 8845 | I.S. | 0.54938 | 8846 | TSC2 | 0.54936 | 8847 | PTEN | 0.54927 |
| 8848 | I.S. | 0.54925 | 8849 | SDHA | 0.54923 | 8850 | I.S. | 0.54923 |
| 8851 | I.S. | 0.54923 | 8852 | TP53 | 0.54912 | 8853 | I.S. | 0.54907 |
| 8854 | ARID1A | 0.54902 | 8855 | DNM2 | 0.549 | 8856 | I.S. | 0.54899 |
| 8857 | DOT1L | 0.54897 | 8858 | NOTCH1 | 0.54891 | 8859 | PLCG2 | 0.54891 |
| 8860 | HOXA11 | 0.54891 | 8861 | PLCG1 | 0.54889 | 8862 | PREX2 | 0.54888 |
| 8863 | MET | 0.54883 | 8864 | I.S. | 0.54873 | 8865 | PBRM1 | 0.54864 |
| 8866 | DDX11 | 0.54855 | 8867 | PIK3CB | 0.54854 | 8868 | BAP1 | 0.54852 |
| 8869 | I.S. | 0.5485 | 8870 | DICER1 | 0.54849 | 8871 | TERT | 0.54849 |
| 8872 | ERBB2 | 0.54849 | 8873 | I.S. | 0.54846 | 8874 | KMT2A | 0.54846 |
| 8875 | I.S. | 0.54841 | 8876 | I.S. | 0.54836 | 8877 | ATG2B | 0.54834 |
| 8878 | I.S. | 0.54832 | 8879 | ATR | 0.54829 | 8880 | FLCN | 0.54828 |
| 8881 | STAT4 | 0.54817 | 8882 | FANCM | 0.54807 | 8883 | I.S. | 0.54806 |
| 8884 | I.S. | 0.54803 | 8885 | I.S. | 0.54802 | 8886 | PRKDC | 0.548 |
| 8887 | DICER1 | 0.54793 | 8888 | SMC3 | 0.54792 | 8889 | | 0.5479 |
| 8890 | I.S. | 0.5479 | 8891 | I.S. | 0.54786 | 8892 | I.S. | 0.54785 |
| 8893 | I.S. | 0.5478 | 8894 | I.S. | 0.54779 | 8895 | KDM5C | 0.54773 |
| 8896 | HOXA11 | 0.54772 | 8897 | I.S. | 0.54766 | 8898 | I.S. | 0.54763 |
| 8899 | I.S. | 0.54762 | 8900 | SPTA1 | 0.54761 | 8901 | ARID2 | 0.54755 |
| 8902 | I.S. | 0.54747 | 8903 | I.S. | 0.54746 | 8904 | I.S. | 0.54739 |
| 8905 | I.S. | 0.54727 | 8906 | I.S. | 0.54724 | 8907 | BRD4 | 0.54715 |
| 8908 | I.S. | 0.5471 | 8909 | CIC | 0.54701 | 8910 | NOTCH1 | 0.54701 |
| 8911 | KMT2D | 0.54692 | 8912 | LYN | 0.54689 | 8913 | PREX2 | 0.54688 |
| 8914 | KMT2A | 0.54687 | 8915 | CREBBP | 0.54683 | 8916 | I.S. | 0.54676 |
| 8917 | SBF2 | 0.54665 | 8918 | ERRFI1 | 0.54662 | 8919 | I.S. | 0.54653 |
| 8920 | I.S. | 0.54653 | 8921 | I.S. | 0.54652 | 8922 | I.S. | 0.54645 |
| 8923 | I.S. | 0.54644 | 8924 | GNAS | 0.54635 | 8925 | I.S. | 0.54633 |
| 8926 | AMER1 | 0.54625 | 8927 | ATM | 0.54621 | 8928 | IDH2, | 0.54619 |
| | | | | | | | IDH2-DT | |
| 8929 | I.S. | 0.54619 | 8930 | ZNF217 | 0.54618 | 8931 | I.S. | 0.54614 |
| 8932 | I.S. | 0.5461 | 8933 | I.S. | 0.5461 | 8934 | ERBB4 | 0.54609 |
| 8935 | I.S. | 0.54606 | 8936 | I.S. | 0.54605 | 8937 | GATA2 | 0.546 |
| 8938 | AKT3 | 0.54597 | 8939 | DICER1 | 0.54593 | 8940 | I.S. | 0.54589 |
| 8941 | AURKA | 0.54583 | 8942 | RBBP6 | 0.54573 | 8943 | I.S. | 0.54571 |
| 8944 | HGF | 0.54567 | 8945 | MEN1 | 0.54567 | 8946 | PIK3R2 | 0.54564 |
| 8947 | I.S. | 0.54559 | 8948 | I.S. | 0.54546 | 8949 | I.S. | 0.54538 |
| 8950 | KIF23 | 0.54535 | 8951 | I.S. | 0.54535 | 8952 | DICER1 | 0.54534 |
| 8953 | I.S. | 0.54532 | 8954 | MUTYH | 0.54529 | 8955 | FGFR2 | 0.54529 |
| 8956 | LYST | 0.54529 | 8957 | I.S. | 0.54529 | 8958 | I.S. | 0.54523 |
| 8959 | I.S. | 0.54523 | 8960 | RTEL1 | 0.54523 | 8961 | I.S. | 0.54522 |
| 8962 | I.S. | 0.54516 | 8963 | I.S. | 0.54515 | 8964 | SAMD9L | 0.54515 |
| 8965 | DNM2 | 0.54505 | 8966 | I.S. | 0.54501 | 8967 | I.S. | 0.54499 |
| 8968 | PDK1 | 0.54499 | 8969 | I.S. | 0.54494 | 8970 | SMARCA4 | 0.54488 |
| 8971 | I.S. | 0.54484 | 8972 | I.S. | 0.54482 | 8973 | CBLB | 0.54473 |
| 8974 | KIT | 0.54461 | 8975 | I.S. | 0.5445 | 8976 | FLT4 | 0.54445 |
| 8977 | I.S. | 0.54443 | 8978 | ETV6 | 0.54431 | 8979 | PIK3C2B | 0.54428 |
| 8980 | RPTOR | 0.54425 | 8981 | CIC | 0.54425 | 8982 | I.S. | 0.54424 |
| 8983 | GID4 | 0.54422 | 8984 | DOT1L | 0.54419 | 8985 | I.S. | 0.54419 |
| 8986 | SBF2 | 0.54419 | 8987 | I.S. | 0.54416 | 8988 | I.S. | 0.54413 |
| 8989 | LPIN2 | 0.5441 | 8990 | RAD54L | 0.54408 | 8991 | MAGI2 | 0.54407 |
| 8992 | HGF | 0.54407 | 8993 | I.S. | 0.54395 | 8994 | I.S. | 0.54391 |
| 8995 | ATG2B | 0.54389 | 8996 | I.S. | 0.54386 | 8997 | I.S. | 0.54381 |
| 8998 | I.S. | 0.5438 | 8999 | FBXW7 | 0.5438 | 9000 | I.S. | 0.54377 |
| 9001 | SUZ12 | 0.54372 | 9002 | I.S. | 0.54371 | 9003 | FUBP1 | 0.54363 |
| 9004 | I.S. | 0.54362 | 9005 | CSF3R | 0.5436 | 9006 | I.S. | 0.54356 |
| 9007 | CALR | 0.54348 | 9008 | RANBP2 | 0.54342 | 9009 | I.S. | 0.54341 |
| 9010 | I.S. | 0.54336 | 9011 | DNM2 | 0.5433 | 9012 | I.S. | 0.54327 |
| 9013 | AMER1 | 0.54326 | 9014 | SAMD9L | 0.54309 | 9015 | PSTPIP1 | 0.54308 |
| 9016 | I.S. | 0.54303 | 9017 | I.S. | 0.54298 | 9018 | I.S. | 0.54295 |
| 9019 | I.S. | 0.54295 | 9020 | I.S. | 0.54294 | 9021 | I.S. | 0.54294 |
| 9022 | FANCM | 0.54293 | 9023 | IKZF3 | 0.54282 | 9024 | NOP10 | 0.5427 |
| 9025 | I.S. | 0.54269 | 9026 | I.S. | 0.54265 | 9027 | ATR | 0.54257 |
| 9028 | I.S. | 0.54252 | 9029 | I.S. | 0.54249 | 9030 | I.S. | 0.54244 |
| 9031 | I.S. | 0.54241 | 9032 | I.S. | 0.54239 | 9033 | I.S. | 0.54234 |
| 9034 | I.S. | 0.54229 | 9035 | TSC2 | 0.54225 | 9036 | MET | 0.54221 |
| 9037 | PIK3R1 | 0.5422 | 9038 | I.S. | 0.54216 | 9039 | I.S. | 0.54213 |
| 9040 | IKZF1 | 0.54212 | 9041 | I.S. | 0.54211 | 9042 | INPP4B | 0.54208 |
| 9043 | NROB1 | 0.54206 | 9044 | I.S. | 0.54205 | 9045 | I.S. | 0.54183 |
| 9046 | I.S. | 0.54182 | 9047 | I.S. | 0.54179 | 9048 | I.S. | 0.54179 |
| 9049 | — | 0.54178 | 9050 | ALK | 0.54176 | 9051 | I.S. | 0.54175 |
| 9052 | LRP1B | 0.54173 | 9053 | SMO | 0.54166 | 9054 | I.S. | 0.54164 |
| 9055 | I.S. | 0.54157 | 9056 | — | 0.54152 | 9057 | I.S. | 0.54151 |
| 9058 | I.S. | 0.54151 | 9059 | I.S. | 0.54145 | 9060 | I.S. | 0.54144 |
| 9061 | I.S. | 0.54141 | 9062 | AXIN1 | 0.5413 | 9063 | I.S. | 0.54124 |
| 9064 | I.S. | 0.5411 | 9065 | I.S. | 0.54099 | 9066 | ANKRD26 | 0.54094 |
| 9067 | I.S. | 0.54092 | 9068 | PDGFRA | 0.5409 | 9069 | ARFRP1 | 0.54078 |
| 9070 | I.S. | 0.54078 | 9071 | MET | 0.54068 | 9072 | I.S. | 0.54066 |
| 9073 | ATR | 0.54063 | 9074 | I.S. | 0.54062 | 9075 | I.S. | 0.54062 |
| 9076 | I.S. | 0.54056 | 9077 | RANBP2 | 0.54051 | 9078 | I.S. | 0.54039 |
| 9079 | NOTCH1 | 0.54035 | 9080 | I.S. | 0.5403 | 9081 | I.S. | 0.5403 |
| 9082 | I.S. | 0.54027 | 9083 | I.S. | 0.54023 | 9084 | I.S. | 0.54011 |
| 9085 | PDGFRA | 0.5401 | 9086 | GNA11 | 0.54 | 9087 | KMT2A | 0.53991 |
| 9088 | I.S. | 0.5399 | 9089 | I.S. | 0.5397 | 9090 | I.S. | 0.53966 |
| 9091 | SPTA1 | 0.53956 | 9092 | I.S. | 0.5394 | 9093 | I.S. | 0.53934 |
| 9094 | SPTA1 | 0.53932 | 9095 | I.S. | 0.53932 | 9096 | NTRK2 | 0.53923 |
| 9097 | MTOR | 0.53923 | 9098 | I.S. | 0.53923 | 9099 | SOX9 | 0.53923 |
| 9100 | EPHA3 | 0.53922 | 9101 | I.S. | 0.53922 | 9102 | LYST | 0.53914 |
| 9103 | I.S. | 0.53907 | 9104 | I.S. | 0.53904 | 9105 | I.S. | 0.53899 |
| 9106 | I.S. | 0.53896 | 9107 | KDM5C | 0.53893 | 9108 | I.S. | 0.5389 |
| 9109 | BCR | 0.53884 | 9110 | I.S. | 0.53883 | 9111 | I.S. | 0.53871 |
| 9112 | I.S. | 0.53868 | 9113 | I.S. | 0.53865 | 9114 | CALR | 0.53864 |
| 9115 | I.S. | 0.53864 | 9116 | I.S. | 0.53854 | 9117 | WAS | 0.53852 |
| 9118 | I.S. | 0.53852 | 9119 | NLRP3 | 0.53839 | 9120 | SPTA1 | 0.53836 |
| 9121 | FGFR2 | 0.53828 | 9122 | PPM1D | 0.53825 | 9123 | TET2 | 0.53811 |
| 9124 | NFE2L2 | 0.538 | 9125 | I.S. | 0.53798 | 9126 | JUN | 0.53792 |
| 9127 | I.S. | 0.53789 | 9128 | MSH2 | 0.53783 | 9129 | I.S. | 0.53772 |
| 9130 | CD79B | 0.53768 | 9131 | FLT4 | 0.53758 | 9132 | SF3B1 | 0.53753 |
| 9133 | SDHA | 0.53746 | 9134 | RBBP6 | 0.53733 | 9135 | FGFR1 | 0.53732 |
| 9136 | I.S. | 0.5373 | 9137 | I.S. | 0.5373 | 9138 | I.S. | 0.5373 |
| 9139 | I.S. | 0.53729 | 9140 | RAD54L | 0.53726 | 9141 | BCR | 0.53721 |
| 9142 | I.S. | 0.53721 | 9143 | I.S. | 0.53718 | 9144 | I.S. | 0.53712 |
| 9145 | I.S. | 0.53709 | 9146 | FOXP1 | 0.53702 | 9147 | I.S. | 0.53702 |
| 9148 | CARD11 | 0.537 | 9149 | I.S. | 0.537 | 9150 | APC | 0.53697 |
| 9151 | I.S. | 0.53689 | 9152 | JATR | 0.53682 | 9153 | NOTCH2 | 0.53678 |
| 9154 | I.S. | 0.53674 | 9155 | I.S. | 0.53672 | 9156 | ATM | 0.53672 |
| 9157 | I.S. | 0.53661 | 9158 | I.S. | 0.53661 | 9159 | TOP1 | 0.53656 |
| 9160 | I.S. | 0.53643 | 9161 | I.S. | 0.5364 | 9162 | I.S. | 0.53632 |
| 9163 | I.S. | 0.53628 | 9164 | I.S. | 0.53625 | 9165 | RNF43 | 0.5362 |
| 9166 | LRP1B | 0.53619 | 9167 | I.S. | 0.53613 | 9168 | I.S. | 0.53611 |
| 9169 | I.S. | 0.53607 | 9170 | WAS | 0.53606 | 9171 | I.S. | 0.53602 |
| 9172 | KMT2D | 0.53602 | 9173 | AXL | 0.53597 | 9174 | I.S. | 0.53593 |
| 9175 | I.S. | 0.53592 | 9176 | FANCI | 0.5359 | 9177 | I.S. | 0.5359 |
| 9178 | I.S. | 0.53587 | 9179 | I.S. | 0.5358 | 9180 | RTEL1 | 0.53576 |
| 9181 | PDK1 | 0.53569 | 9182 | I.S. | 0.53567 | 9183 | I.S. | 0.53562 |
| 9184 | I.S. | 0.53559 | 9185 | I.S. | 0.53551 | 9186 | GALNT12 | 0.53549 |
| 9187 | PAX5 | 0.53545 | 9188 | I.S. | 0.5354 | 9189 | I.S. | 0.53537 |
| 9190 | I.S. | 0.53537 | 9191 | CTNNB1 | 0.53536 | 9192 | STAT3 | 0.53536 |
| 9193 | I.S. | 0.53527 | 9194 | I.S. | 0.53522 | 9195 | SMO | 0.53519 |
| 9196 | ATM | 0.53513 | 9197 | LYST | 0.5351 | 9198 | RPTOR | 0.53509 |
| 9199 | NTRK2 | 0.53504 | 9200 | I.S. | 0.53503 | 9201 | I.S. | 0.53501 |
| 9202 | I.S. | 0.535 | 9203 | ATM | 0.53494 | 9204 | I.S. | 0.53492 |
| 9205 | I.S. | 0.53492 | 9206 | I.S. | 0.53476 | 9207 | I.S. | 0.5347 |
| 9208 | I.S. | 0.5347 | 9209 | FUBP1 | 0.53468 | 9210 | I.S. | 0.53468 |
| 9211 | NOTCH1 | 0.53463 | 9212 | CRLF2 | 0.53461 | 9213 | SMC1A | 0.53458 |
| 9214 | SBF2 | 0.53456 | 9215 | — | 0.53454 | 9216 | I.S. | 0.53451 |
| 9217 | I.S. | 0.53451 | 9218 | PRKDC | 0.53445 | 9219 | ABL2 | 0.5343 |
| 9220 | I.S. | 0.53426 | 9221 | NLRP3 | 0.53425 | 9222 | MET | 0.53418 |
| 9223 | GSK3B | 0.53415 | 9224 | RAF1 | 0.53415 | 9225 | I.S. | 0.53414 |
| 9226 | I.S. | 0.53403 | 9227 | CBLB | 0.53403 | 9228 | GRM3 | 0.53403 |
| 9229 | I.S. | 0.53401 | 9230 | PIK3CA | 0.53398 | 9231 | I.S. | 0.53389 |
| 9232 | I.S. | 0.53386 | 9233 | SLIT2 | 0.53381 | 9234 | I.S. | 0.53377 |
| 9235 | ARAF | 0.53374 | 9236 | SMARCA4 | 0.53353 | 9237 | I.S. | 0.5335 |
| 9238 | NSD2 | 0.53343 | 9239 | I.S. | 0.53343 | 9240 | PALB2 | 0.53343 |
| 9241 | PTPN11 | 0.5334 | 9242 | I.S. | 0.53329 | 9243 | I.S. | 0.53328 |
| 9244 | GNAS | 0.53326 | 9245 | I.S. | 0.53325 | 9246 | BCR | 0.53322 |
| 9247 | I.S. | 0.53322 | 9248 | I.S. | 0.53318 | 9249 | I.S. | 0.53306 |
| 9250 | ARID1B | 0.53306 | 9251 | DOT1L | 0.53294 | 9252 | NTRK2 | 0.53294 |
| 9253 | I.S. | 0.53292 | 9254 | RAD50 | 0.5329 | 9255 | DICER1 | 0.53286 |
| 9256 | CREBBP | 0.53286 | 9257 | FANCA | 0.53286 | 9258 | I.S. | 0.53282 |
| 9259 | I.S. | 0.53279 | 9260 | I.S. | 0.53277 | 9261 | I.S. | 0.53275 |
| 9262 | I.S. | 0.53274 | 9263 | I.S. | 0.53272 | 9264 | ERBB3 | 0.53266 |
| 9265 | RICTOR | 0.53263 | 9266 | ERBB2 | 0.53261 | 9267 | SMAD3 | 0.5326 |
| 9268 | NOTCH2 | 0.53255 | 9269 | I.S. | 0.53254 | 9270 | ARID1A | 0.53254 |
| 9271 | I.S. | 0.53249 | 9272 | I.S. | 0.53246 | 9273 | RANBP2 | 0.53233 |
| 9274 | HRAS | 0.53233 | 9275 | SBDS | 0.53229 | 9276 | SETD2 | 0.53228 |
| 9277 | I.S. | 0.53227 | 9278 | NBPF20, | 0.53223 | 9279 | FANCC | 0.5322 |
| | | | | NBPF19, | | | | |
| | | | | NBPF10, | | | | |
| | | | | RBM8A | | | | |
| 9280 | SMARCA4 | 0.53216 | 9281 | I.S. | 0.53213 | 9282 | SDHA | 0.53209 |
| 9283 | I.S. | 0.53209 | 9284 | MUTYH | 0.53208 | 9285 | I.S. | 0.53205 |
| 9286 | CTNNA1 | 0.53203 | 9287 | I.S. | 0.53203 | 9288 | TOP1 | 0.53196 |
| 9289 | PBRM1 | 0.53195 | 9290 | KDR | 0.53192 | 9291 | I.S. | 0.53189 |
| 9292 | I.S. | 0.53189 | 9293 | G6PC3 | 0.53189 | 9294 | I.S. | 0.53189 |
| 9295 | MEFV | 0.53181 | 9296 | I.S. | 0.5318 | 9297 | NPM1 | 0.5318 |
| 9298 | I.S. | 0.53172 | 9299 | CDC73 | 0.5317 | 9300 | I.S. | 0.53167 |
| 9301 | SBF2 | 0.53159 | 9302 | I.S. | 0.53157 | 9303 | EGFR | 0.53155 |
| 9304 | I.S. | 0.53151 | 9305 | FLT3 | 0.53151 | 9306 | JAK3 | 0.5315 |
| 9307 | I.S. | 0.53149 | 9308 | I.S. | 0.53143 | 9309 | I.S. | 0.53141 |
| 9310 | I.S. | 0.53137 | 9311 | STAT3 | 0.53136 | 9312 | I.S. | 0.53129 |
| 9313 | MTOR | 0.53127 | 9314 | I.S. | 0.53126 | 9315 | TOP1 | 0.53124 |
| 9316 | I.S. | 0.53117 | 9317 | I.S. | 0.53117 | 9318 | I.S. | 0.53108 |
| 9319 | I.S. | 0.531 | 9320 | I.S. | 0.53096 | 9321 | SMC1A | 0.53091 |
| 9322 | I.S. | 0.53088 | 9323 | I.S. | 0.53082 | 9324 | PREX2 | 0.53076 |
| 9325 | FANCD2 | 0.53074 | 9326 | JAK3 | 0.53064 | 9327 | I.S. | 0.53053 |
| 9328 | I.S. | 0.53045 | 9329 | I.S. | 0.53036 | 9330 | 1 | 0.53034 |
| 9331 | I.S. | 0.53034 | 9332 | I.S. | 0.53031 | 9333 | ANKRD26 | 0.53023 |
| 9334 | DDX41 | 0.53019 | 9335 | IL2RG | 0.53017 | 9336 | I.S. | 0.53011 |
| 9337 | MET | 0.53011 | 9338 | RB1 | 0.52999 | 9339 | I.S. | 0.52997 |
| 9340 | I.S. | 0.52996 | 9341 | POLE | 0.52994 | 9342 | I.S. | 0.5299 |
| 9343 | I.S. | 0.52987 | 9344 | I.S. | 0.52986 | 9345 | CDH1 | 0.52986 |
| 9346 | SMC1A | 0.52985 | 9347 | I.S. | 0.5298 | 9348 | JUN | 0.52978 |
| 9349 | AKT1 | 0.52978 | 9350 | I.S. | 0.52964 | 9351 | NBN | 0.5296 |
| 9352 | I.S. | 0.52954 | 9353 | I.S. | 0.52952 | 9354 | I.S. | 0.5295 |
| 9355 | I.S. | 0.52948 | 9356 | I.S. | 0.52942 | 9357 | I.S. | 0.52935 |
| 9358 | TERT | 0.52933 | 9359 | I.S. | 0.52919 | 9360 | MEFV | 0.5291 |
| 9361 | I.S. | 0.52908 | 9362 | I.S. | 0.52907 | 9363 | I.S. | 0.52903 |
| 9364 | STAT3 | 0.52899 | 9365 | I.S. | 0.52894 | 9366 | ARID1A | 0.52891 |
| 9367 | I.S. | 0.52873 | 9368 | LRP1B | 0.52862 | 9369 | I.S. | 0.52859 |
| 9370 | I.S. | 0.52854 | 9371 | I.S. | 0.5285 | 9372 | CRLF2 | 0.52842 |
| 9373 | SPEN | 0.52839 | 9374 | RAD50, | 0.52838 | 9375 | I.S. | 0.52836 |
| | | | | TH2LCRR | | | | |
| 9376 | I.S. | 0.52836 | 9377 | BRD4 | 0.52831 | 9378 | SMC1A | 0.52824 |
| 9379 | I.S. | 0.5282 | 9380 | ATG2B | 0.5282 | 9381 | MAPK1 | 0.52818 |
| 9382 | PAX5 | 0.52817 | 9383 | PAK3 | 0.5281 | 9384 | NOTCH2 | 0.52806 |
| 9385 | I.S. | 0.52797 | 9386 | I.S. | 0.52796 | 9387 | I.S. | 0.52792 |
| 9388 | I.S. | 0.5279 | 9389 | I.S. | 0.52781 | 9390 | MED12 | 0.52774 |
| 9391 | RPTOR | 0.52761 | 9392 | I.S. | 0.52757 | 9393 | BCL2L1 | 0.52757 |
| 9394 | KDM5C | 0.52754 | 9395 | KMT2D | 0.52739 | 9396 | I.S. | 0.52717 |
| 9397 | I.S. | 0.52715 | 9398 | I.S. | 0.52711 | 9399 | BCORL1 | 0.527 |
| 9400 | I.S. | 0.52699 | 9401 | FBXW7 | 0.52696 | 9402 | I.S. | 0.52695 |
| 9403 | FANCE | 0.52693 | 9404 | I.S. | 0.52692 | 9405 | I.S. | 0.52681 |
| 9406 | MET | 0.52681 | 9407 | I.S. | 0.52677 | 9408 | EGFR | 0.52677 |
| 9409 | SEC23B | 0.52675 | 9410 | I.S. | 0.52672 | 9411 | I.S. | 0.52669 |
| 9412 | I.S. | 0.52666 | 9413 | I.S. | 0.52661 | 9414 | I.S. | 0.52658 |
| 9415 | I.S. | 0.52648 | 9416 | I.S. | 0.52643 | 9417 | I.S. | 0.52632 |
| 9418 | I.S. | 0.52613 | 9419 | I.S. | 0.52602 | 9420 | I.S. | 0.52602 |
| 9421 | ATR | 0.52599 | 9422 | DKC1 | 0.52596 | 9423 | I.S. | 0.52592 |
| 9424 | I.S. | 0.52591 | 9425 | RANBP2 | 0.52586 | 9426 | I.S. | 0.52583 |
| 9427 | EPHA3 | 0.52583 | 9428 | NOTCH2 | 0.52574 | 9429 | PREX2 | 0.52574 |
| 9430 | SMARCA4 | 0.52574 | 9431 | I.S. | 0.52571 | 9432 | GEN1 | 0.52571 |
| 9433 | KDR | 0.52568 | 9434 | FANCI | 0.52564 | 9435 | LYN | 0.52562 |
| 9436 | I.S. | 0.5255 | 9437 | I.S. | 0.52548 | 9438 | I.S. | 0.52542 |
| 9439 | BRCA1 | 0.52535 | 9440 | IRS2 | 0.52534 | 9441 | MAGI2 | 0.52533 |
| 9442 | I.S. | 0.5253 | 9443 | I.S. | 0.5253 | 9444 | FANCC | 0.52529 |
| 9445 | I.S. | 0.52519 | 9446 | I.S. | 0.52516 | 9447 | CHD2 | 0.52514 |
| 9448 | I.S. | 0.52507 | 9449 | FANCB | 0.52503 | 9450 | I.S. | 0.52501 |
| 9451 | I.S. | 0.525 | 9452 | FANCB | 0.52495 | 9453 | SF3B1 | 0.52494 |
| 9454 | I.S. | 0.52494 | 9455 | CUX1 | 0.52493 | 9456 | PDK1 | 0.52491 |
| 9457 | I.S. | 0.52491 | 9458 | FRS2 | 0.5249 | 9459 | I.S. | 0.52488 |
| 9460 | I.S. | 0.52486 | 9461 | I.S. | 0.52484 | 9462 | I.S. | 0.52479 |
| 9463 | I.S. | 0.52474 | 9464 | NOTCH2 | 0.5247 | 9465 | NTRK2 | 0.52462 |
| 9466 | I.S. | 0.52461 | 9467 | FOXP1 | 0.52452 | 9468 | ASXL1 | 0.52449 |
| 9469 | BCYRN1, | 0.52438 | 9470 | KMT2D | 0.52437 | 9471 | I.S. | 0.52435 |
| | TAF1 | | | | | | | |
| 9472 | HGF | 0.52417 | 9473 | SMARCB1 | 0.52413 | 9474 | I.S. | 0.52412 |
| 9475 | SF3B1 | 0.52411 | 9476 | ADA | 0.52409 | 9477 | I.S. | 0.52407 |
| 9478 | I.S. | 0.52406 | 9479 | IDH2 | 0.52401 | 9480 | FANCA | 0.52398 |
| 9481 | PRSS1 | 0.52393 | 9482 | GEN1 | 0.52393 | 9483 | MAP2K1 | 0.52388 |
| 9484 | I.S. | 0.52381 | 9485 | I.S. | 0.5238 | 9486 | I.S. | 0.52373 |
| 9487 | I.S. | 0.52371 | 9488 | MDM4 | 0.52371 | 9489 | I.S. | 0.52367 |
| 9490 | I.S. | 0.52366 | 9491 | I.S. | 0.52364 | 9492 | I.S. | 0.5236 |
| 9493 | I.S. | 0.52356 | 9494 | GABRA6 | 0.52354 | 9495 | I.S. | 0.52351 |
| 9496 | I.S. | 0.52351 | 9497 | I.S. | 0.52345 | 9498 | I.S. | 0.52343 |
| 9499 | RAD51C | 0.52341 | 9500 | ROS1 | 0.5234 | 9501 | I.S. | 0.5234 |
| 9502 | I.S. | 0.52338 | 9503 | MET | 0.5233 | 9504 | CDK12 | 0.52326 |
| 9505 | I.S. | 0.52325 | 9506 | I.S. | 0.52317 | 9507 | I.S. | 0.52315 |
| 9508 | SBF2 | 0.52312 | 9509 | I.S. | 0.52308 | 9510 | I.S. | 0.52308 |
| 9511 | I.S. | 0.52302 | 9512 | I.S. | 0.52299 | 9513 | I.S. | 0.52298 |
| 9514 | HGF | 0.52283 | 9515 | I.S. | 0.52276 | 9516 | I.S. | 0.52272 |
| 9517 | I.S. | 0.5227 | 9518 | RANBP2 | 0.52267 | 9519 | I.S. | 0.52266 |
| 9520 | RTEL1 | 0.52266 | 9521 | I.S. | 0.52262 | 9522 | I.S. | 0.52262 |
| 9523 | I.S. | 0.52256 | 9524 | MTOR | 0.52244 | 9525 | I.S. | 0.52237 |
| 9526 | I.S. | 0.52237 | 9527 | DICER1 | 0.52227 | 9528 | I.S. | 0.52227 |
| 9529 | TAL1 | 0.52223 | 9530 | I.S. | 0.52218 | 9531 | NF1 | 0.52215 |
| 9532 | I.S. | 0.52214 | 9533 | NOTCH2 | 0.52213 | 9534 | FANCA | 0.52212 |
| 9535 | ATM | 0.52194 | 9536 | I.S. | 0.52188 | 9537 | ATM | 0.52186 |
| 9538 | I.S. | 0.52183 | 9539 | I.S. | 0.52158 | 9540 | I.S. | 0.52158 |
| 9541 | CTC1 | 0.52154 | 9542 | I.S. | 0.52152 | 9543 | I.S. | 0.52141 |
| 9544 | I.S. | 0.52138 | 9545 | I.S. | 0.52138 | 9546 | I.S. | 0.52134 |
| 9547 | I.S. | 0.52132 | 9548 | DNMT3A | 0.52128 | 9549 | I.S. | 0.5212 |
| 9550 | I.S. | 0.52116 | 9551 | I.S. | 0.52105 | 9552 | I.S. | 0.52104 |
| 9553 | DICER1 | 0.52096 | 9554 | SPEN | 0.52096 | 9555 | I.S. | 0.52081 |
| 9556 | PIK3CA | 0.52081 | 9557 | I.S. | 0.52081 | 9558 | I.S. | 0.52076 |
| 9559 | I.S. | 0.52065 | 9560 | I.S. | 0.52064 | 9561 | I.S. | 0.52052 |
| 9562 | I.S. | 0.52051 | 9563 | SMARCA4 | 0.52051 | 9564 | I.S. | 0.52044 |
| 9565 | FANCI | 0.5204 | 9566 | I.S. | 0.52031 | 9567 | I.S. | 0.52031 |
| 9568 | I.S. | 0.52027 | 9569 | TET2 | 0.52007 | 9570 | I.S. | 0.52006 |
| 9571 | I.S. | 0.52004 | 9572 | MSH2 | 0.51995 | 9573 | I.S. | 0.51989 |
| 9574 | I.S. | 0.51979 | 9575 | I.S. | 0.51973 | 9576 | I.S. | 0.51971 |
| 9577 | FANCD2 | 0.51966 | 9578 | PIK3CA | 0.51959 | 9579 | BCR | 0.51956 |
| 9580 | KMT2B | 0.51954 | 9581 | I.S. | 0.51947 | 9582 | BRIP1 | 0.51941 |
| 9583 | I.S. | 0.51941 | 9584 | I.S. | 0.51941 | 9585 | PIK3R2 | 0.51935 |
| 9586 | CDK6 | 0.51931 | 9587 | I.S. | 0.5193 | 9588 | I.S. | 0.51927 |
| 9589 | I.S. | 0.51918 | 9590 | MRE11 | 0.51912 | 9591 | SETD2 | 0.51909 |
| 9592 | I.S. | 0.51909 | 9593 | I.S. | 0.51907 | 9594 | — | 0.51906 |
| 9595 | I.S. | 0.51903 | 9596 | KMT2B | 0.51901 | 9597 | I.S. | 0.5189 |
| 9598 | I.S. | 0.5189 | 9599 | NOTCH3 | 0.51885 | 9600 | SRSF2, | 0.51884 |
| | | | | | | | MFSD11 | |
| 9601 | I.S. | 0.51884 | 9602 | I.S. | 0.51877 | 9603 | I.S. | 0.51876 |
| 9604 | I.S. | 0.51873 | 9605 | CHD2 | 0.51855 | 9606 | I.S. | 0.51854 |
| 9607 | I.S. | 0.51849 | 9608 | I.S. | 0.51849 | 9609 | I.S. | 0.51846 |
| 9610 | I.S. | 0.51818 | 9611 | I.S. | 0.51817 | 9612 | FANCI | 0.51808 |
| 9613 | FLCN | 0.51805 | 9614 | I.S. | 0.51802 | 9615 | CARD11 | 0.5178 |
| 9616 | I.S. | 0.51775 | 9617 | I.S. | 0.51771 | 9618 | RAD51 | 0.51762 |
| 9619 | ARID1A | 0.51754 | 9620 | I.S. | 0.51751 | 9621 | FGFR2 | 0.5174 |
| 9622 | GRIN2A | 0.5174 | 9623 | I.S. | 0.5173 | 9624 | KEL | 0.51728 |
| 9625 | FGFR3 | 0.51727 | 9626 | I.S. | 0.51715 | 9627 | I.S. | 0.51715 |
| 9628 | FLT1 | 0.51712 | 9629 | I.S. | 0.51709 | 9630 | I.S. | 0.51698 |
| 9631 | I.S. | 0.51686 | 9632 | ATG2B | 0.51685 | 9633 | I.S. | 0.51683 |
| 9634 | I.S. | 0.51678 | 9635 | ERG | 0.51677 | 9636 | I.S. | 0.51673 |
| 9637 | GATA2 | 0.51673 | 9638 | I.S. | 0.51668 | 9639 | I.S. | 0.51664 |
| 9640 | I.S. | 0.51664 | 9641 | ATM | 0.51663 | 9642 | DNMT3A | 0.51657 |
| 9643 | KDM5C | 0.51657 | 9644 | I.S. | 0.51653 | 9645 | B2M | 0.51653 |
| 9646 | MPL | 0.51651 | 9647 | I.S. | 0.51641 | 9648 | I.S. | 0.51641 |
| 9649 | I.S. | 0.5164 | 9650 | I.S. | 0.51639 | 9651 | I.S. | 0.51638 |
| 9652 | GRM3 | 0.51638 | 9653 | ADA | 0.51638 | 9654 | I.S. | 0.51638 |
| 9655 | I.S. | 0.51627 | 9656 | I.S. | 0.51626 | 9657 | I.S. | 0.51617 |
| 9658 | I.S. | 0.51616 | 9659 | I.S. | 0.51616 | 9660 | FLT1 | 0.51611 |
| 9661 | NOTCH1 | 0.5161 | 9662 | BIRC3 | 0.51606 | 9663 | I.S. | 0.51603 |
| 9664 | I.S. | 0.51601 | 9665 | RAD51B | 0.516 | 9666 | I.S. | 0.51598 |
| 9667 | I.S. | 0.51591 | 9668 | I.S. | 0.51588 | 9669 | GFI1 | 0.51588 |
| 9670 | SAMD9L | 0.51585 | 9671 | ATG2B | 0.51583 | 9672 | I.S. | 0.51582 |
| 9673 | I.S. | 0.5158 | 9674 | BRIP1 | 0.51569 | 9675 | PPM1D | 0.51567 |
| 9676 | I.S. | 0.51564 | 9677 | KDR | 0.51562 | 9678 | I.S. | 0.51558 |
| 9679 | RAD51C | 0.51556 | 9680 | RTEL1 | 0.51552 | 9681 | TCIRG1 | 0.51552 |
| 9682 | NOTCH1 | 0.51543 | 9683 | I.S. | 0.5154 | 9684 | I.S. | 0.51531 |
| 9685 | I.S. | 0.51529 | 9686 | I.S. | 0.51525 | 9687 | I.S. | 0.51519 |
| 9688 | BARD1 | 0.51513 | 9689 | I.S. | 0.51504 | 9690 | LPIN2 | 0.51504 |
| 9691 | I.S. | 0.51498 | 9692 | I.S. | 0.51487 | 9693 | I.S. | 0.51484 |
| 9694 | RBBP6 | 0.51477 | 9695 | KDR | 0.51469 | 9696 | MET | 0.51467 |
| 9697 | I.S. | 0.51466 | 9698 | I.S. | 0.51466 | 9699 | XPO1 | 0.51455 |
| 9700 | BRCA1 | 0.51455 | 9701 | NF2 | 0.51447 | 9702 | JATR | 0.51439 |
| 9703 | MPL | 0.51431 | 9704 | I.S. | 0.51421 | 9705 | I.S. | 0.51419 |
| 9706 | FANCI | 0.51397 | 9707 | FGFR1 | 0.51392 | 9708 | I.S. | 0.5139 |
| 9709 | LRP1B | 0.5138 | 9710 | I.S. | 0.51379 | 9711 | KMT2A | 0.51374 |
| 9712 | I.S. | 0.51374 | 9713 | I.S. | 0.51367 | 9714 | I.S. | 0.51359 |
| 9715 | PRKDC | 0.51357 | 9716 | PIK3CA | 0.5135 | 9717 | AKT1 | 0.51348 |
| 9718 | RANBP2 | 0.51344 | 9719 | I.S. | 0.51343 | 9720 | RAC1 | 0.51338 |
| 9721 | CDC73 | 0.51338 | 9722 | I.S. | 0.51338 | 9723 | I.S. | 0.51338 |
| 9724 | I.S. | 0.51337 | 9725 | PMS1 | 0.51335 | 9726 | I.S. | 0.51326 |
| 9727 | I.S. | 0.51319 | 9728 | I.S. | 0.51318 | 9729 | I.S. | 0.51318 |
| 9730 | I.S. | 0.51317 | 9731 | I.S. | 0.51311 | 9732 | I.S. | 0.51309 |
| 9733 | KIT | 0.51307 | 9734 | KEL | 0.51303 | 9735 | I.S. | 0.51302 |
| 9736 | TOP2A | 0.51302 | 9737 | I.S. | 0.5129 | 9738 | TGFBR2 | 0.51287 |
| 9739 | KMT2A | 0.51285 | 9740 | KDR | 0.51282 | 9741 | FUBP1 | 0.51271 |
| 9742 | PDGFRA | 0.51258 | 9743 | I.S. | 0.51254 | 9744 | I.S. | 0.51252 |
| 9745 | ERRFI1 | 0.51246 | 9746 | SMARCA4 | 0.51243 | 9747 | TOP1 | 0.51233 |
| 9748 | BRAF | 0.5123 | 9749 | I.S. | 0.51228 | 9750 | ATM | 0.51228 |
| 9751 | NUP93 | 0.51215 | 9752 | I.S. | 0.51208 | 9753 | I.S. | 0.51205 |
| 9754 | I.S. | 0.51202 | 9755 | JAK3 | 0.51199 | 9756 | CDK12 | 0.51198 |
| 9757 | FOXL2 | 0.51198 | 9758 | RICTOR | 0.51192 | 9759 | I.S. | 0.51183 |
| 9760 | XPO1 | 0.51181 | 9761 | I.S. | 0.51176 | 9762 | I.S. | 0.51165 |
| 9763 | JAK1 | 0.51163 | 9764 | I.S. | 0.51162 | 9765 | WAS | 0.51161 |
| 9766 | NSD1 | 0.51157 | 9767 | I.S. | 0.51157 | 9768 | I.S. | 0.51157 |
| 9769 | NOTCH3 | 0.51146 | 9770 | DKC1 | 0.51143 | 9771 | TERT | 0.51142 |
| 9772 | I.S. | 0.51135 | 9773 | I.S. | 0.51132 | 9774 | SBF2 | 0.51128 |
| 9775 | FGF10 | 0.51124 | 9776 | I.S. | 0.51122 | 9777 | KMT2C | 0.51116 |
| 9778 | BCYRN1, | 0.51116 | 9779 | I.S. | 0.51108 | 9780 | PIK3R2 | 0.51103 |
| | TAF1 | | | | | | | |
| 9781 | I.S. | 0.51103 | 9782 | SPOP | 0.51101 | 9783 | I.S. | 0.51099 |
| 9784 | I.S. | 0.51083 | 9785 | I.S. | 0.51078 | 9786 | I.S. | 0.51077 |
| 9787 | ERBB3 | 0.51072 | 9788 | ADGRA2 | 0.51071 | 9789 | RNF43 | 0.51068 |
| 9790 | I.S. | 0.51067 | 9791 | I.S. | 0.51056 | 9792 | I.S. | 0.51052 |
| 9793 | NHP2 | 0.51044 | 9794 | I.S. | 0.51038 | 9795 | I.S. | 0.51034 |
| 9796 | I.S. | 0.5103 | 9797 | FANCI | 0.51029 | 9798 | I.S. | 0.51029 |
| 9799 | I.S. | 0.51018 | 9800 | I.S. | 0.51013 | 9801 | I.S. | 0.51008 |
| 9802 | FANCD2 | 0.50999 | 9803 | I.S. | 0.5099 | 9804 | AK2 | 0.50987 |
| 9805 | SPTA1 | 0.50982 | 9806 | I.S. | 0.50974 | 9807 | I.S. | 0.50973 |
| 9808 | I.S. | 0.50961 | 9809 | I.S. | 0.5096 | 9810 | I.S. | 0.50956 |
| 9811 | I.S. | 0.50949 | 9812 | I.S. | 0.50941 | 9813 | MAP3K1 | 0.5094 |
| 9814 | I.S. | 0.50936 | 9815 | I.S. | 0.50934 | 9816 | FAT1 | 0.50926 |
| 9817 | GSK3B | 0.50924 | 9818 | I.S. | 0.50923 | 9819 | KMT2B | 0.5092 |
| 9820 | EP300 | 0.5092 | 9821 | PIK3CA | 0.50916 | 9822 | I.S. | 0.50912 |
| 9823 | I.S. | 0.50908 | 9824 | KMT2D | 0.50907 | 9825 | I.S. | 0.50904 |
| 9826 | I.S. | 0.50902 | 9827 | SPEN | 0.50901 | 9828 | I.S. | 0.50893 |
| 9829 | I.S. | 0.50887 | 9830 | I.S. | 0.50884 | 9831 | NOTCH2 | 0.50878 |
| 9832 | I.S. | 0.50877 | 9833 | I.S. | 0.50873 | 9834 | TERT | 0.50872 |
| 9835 | I.S. | 0.50867 | 9836 | NOTCH2 | 0.5086 | 9837 | I.S. | 0.50845 |
| 9838 | I.S. | 0.50845 | 9839 | I.S. | 0.50845 | 9840 | I.S. | 0.50841 |
| 9841 | TCF3 | 0.50833 | 9842 | I.S. | 0.50828 | 9843 | RAD51 | 0.50816 |
| 9844 | SETD2 | 0.50813 | 9845 | I.S. | 0.50803 | 9846 | I.S. | 0.50801 |
| 9847 | I.S. | 0.50791 | 9848 | RANBP2 | 0.50784 | 9849 | GLI1 | 0.50783 |
| 9850 | KDM5A | 0.5078 | 9851 | I.S. | 0.50777 | 9852 | SMARCA4 | 0.50775 |
| 9853 | I.S. | 0.50771 | 9854 | I.S. | 0.50771 | 9855 | SOX9 | 0.50768 |
| 9856 | FLT3 | 0.50765 | 9857 | I.S. | 0.50763 | 9858 | I.S. | 0.50759 |
| 9859 | I.S. | 0.50758 | 9860 | KEAP1 | 0.5075 | 9861 | MTOR | 0.50732 |
| 9862 | LYST | 0.50732 | 9863 | EPHA3 | 0.50724 | 9864 | PIM1 | 0.50722 |
| 9865 | I.S. | 0.50717 | 9866 | MAGI2 | 0.50717 | 9867 | FLT1 | 0.50709 |
| 9868 | I.S. | 0.50706 | 9869 | I.S. | 0.50694 | 9870 | MTOR | 0.50691 |
| 9871 | CD274 | 0.50691 | 9872 | I.S. | 0.50686 | 9873 | I.S. | 0.50679 |
| 9874 | I.S. | 0.50679 | 9875 | I.S. | 0.50676 | 9876 | PDGFRA | 0.50674 |
| 9877 | I.S. | 0.50666 | 9878 | I.S. | 0.50666 | 9879 | I.S. | 0.5066 |
| 9880 | AR | 0.50656 | 9881 | I.S. | 0.50652 | 9882 | BIRC3 | 0.50638 |
| 9883 | I.S. | 0.50631 | 9884 | NOTCH2 | 0.50628 | 9885 | I.S. | 0.50626 |
| 9886 | I.S. | 0.50624 | 9887 | I.S. | 0.50622 | 9888 | RANBP2 | 0.5062 |
| 9889 | I.S. | 0.50619 | 9890 | KAT6A | 0.50617 | 9891 | I.S. | 0.50615 |
| 9892 | CDAN1 | 0.50611 | 9893 | I.S. | 0.50611 | 9894 | I.S. | 0.50609 |
| 9895 | I.S. | 0.50595 | 9896 | I.S. | 0.50588 | 9897 | CDKN2A | 0.50587 |
| 9898 | I.S. | 0.50587 | 9899 | NOTCH3 | 0.50584 | 9900 | PIK3CA | 0.50584 |
| 9901 | POLE | 0.50578 | 9902 | I.S. | 0.50578 | 9903 | I.S. | 0.50577 |
| 9904 | I.S. | 0.50575 | 9905 | I.S. | 0.50575 | 9906 | B2M | 0.50571 |
| 9907 | I.S. | 0.50569 | 9908 | AXIN1 | 0.5056 | 9909 | I.S. | 0.50557 |
| 9910 | I.S. | 0.50552 | 9911 | I.S. | 0.5055 | 9912 | RANBP2 | 0.50549 |
| 9913 | I.S. | 0.50549 | 9914 | I.S. | 0.50547 | 9915 | I.S. | 0.50546 |
| 9916 | I.S. | 0.50544 | 9917 | I.S. | 0.50541 | 9918 | NTRK3 | 0.50533 |
| 9919 | NF1 | 0.50532 | 9920 | AMER1 | 0.50525 | 9921 | STAG2 | 0.50522 |
| 9922 | I.S. | 0.50519 | 9923 | I.S. | 0.50518 | 9924 | I.S. | 0.50517 |
| 9925 | TET2 | 0.5051 | 9926 | I.S. | 0.50509 | 9927 | STAT4 | 0.50506 |
| 9928 | I.S. | 0.50501 | 9929 | CDKN1B | 0.505 | 9930 | DDX11 | 0.505 |
| 9931 | MET | 0.50492 | 9932 | TET2 | 0.50468 | 9933 | FLT4 | 0.50468 |
| 9934 | I.S. | 0.50452 | 9935 | I.S. | 0.50451 | 9936 | I.S. | 0.50447 |
| 9937 | I.S. | 0.50423 | 9938 | NOTCH1 | 0.50421 | 9939 | I.S. | 0.50415 |
| 9940 | RAD21 | 0.50409 | 9941 | ERRFI1 | 0.50403 | 9942 | I.S. | 0.50396 |
| 9943 | RUNX1 | 0.5039 | 9944 | PDGFRB | 0.50388 | 9945 | I.S. | 0.50388 |
| 9946 | CTNNA1 | 0.50387 | 9947 | RAD54L | 0.50368 | 9948 | I.S. | 0.50367 |
| 9949 | CDK8 | 0.50367 | 9950 | I.S. | 0.50364 | 9951 | BCL6 | 0.50349 |
| 9952 | I.S. | 0.50346 | 9953 | I.S. | 0.50343 | 9954 | I.S. | 0.5034 |
| 9955 | VHL | 0.50338 | 9956 | FGF4 | 0.50335 | 9957 | I.S. | 0.50335 |
| 9958 | I.S. | 0.50326 | 9959 | SBF2 | 0.50326 | 9960 | MPL | 0.5032 |
| 9961 | I.S. | 0.50319 | 9962 | CDKN1B | 0.50317 | 9963 | EED | 0.50316 |
| 9964 | CHD2 | 0.50316 | 9965 | I.S. | 0.50316 | 9966 | I.S. | 0.50302 |
| 9967 | POLE | 0.50299 | 9968 | FANCB | 0.50297 | 9969 | MEF2B | 0.50295 |
| 9970 | I.S. | 0.50295 | 9971 | I.S. | 0.50292 | 9972 | EPHA3 | 0.5029 |
| 9973 | I.S. | 0.50289 | 9974 | RAD54L | 0.50288 | 9975 | I.S. | 0.50287 |
| 9976 | KDM5C | 0.50284 | 9977 | I.S. | 0.50275 | 9978 | I.S. | 0.50273 |
| 9979 | PRKAR1A | 0.5027 | 9980 | ASXL1 | 0.50267 | 9981 | I.S. | 0.50261 |
| 9982 | ARID1B | 0.50257 | 9983 | DKC1 | 0.50234 | 9984 | SPEN | 0.50234 |
| 9985 | I.S. | 0.50234 | 9986 | PIK3CB | 0.50233 | 9987 | I.S. | 0.5023 |
| 9988 | KMT2D | 0.5023 | 9989 | STAT4 | 0.50221 | 9990 | I.S. | 0.50217 |
| 9991 | XPO1 | 0.5021 | 9992 | I.S. | 0.50207 | 9993 | I.S. | 0.50204 |
| 9994 | I.S. | 0.50201 | 9995 | I.S. | 0.502 | 9996 | MRE11 | 0.50198 |
| 9997 | RET | 0.50194 | 9998 | I.S. | 0.50188 | 9999 | SMC3 | 0.50188 |
| 10000 | RAD21 | 0.50187 | 10001 | U2AF1, | 0.50186 | 10002 | I.S. | 0.50174 |
| | | | | U2AF1L5 | | | | |
| 10003 | I.S. | 0.50174 | 10004 | I.S. | 0.50171 | 10005 | CRLF2 | 0.50163 |
| 10006 | I.S. | 0.50156 | 10007 | I.S. | 0.50155 | 10008 | I.S. | 0.50151 |
| 10009 | I.S. | 0.50149 | 10010 | AR | 0.50148 | 10011 | I.S. | 0.50147 |
| 10012 | STAG2 | 0.50144 | 10013 | I.S. | 0.50144 | 10014 | XRCC3 | 0.50141 |
| 10015 | I.S. | 0.50138 | 10016 | I.S. | 0.5012 | 10017 | I.S. | 0.5012 |
| 10018 | ERBB4 | 0.50097 | 10019 | I.S. | 0.50095 | 10020 | I.S. | 0.50076 |
| 10021 | I.S. | 0.50076 | 10022 | FLT4 | 0.50072 | 10023 | I.S. | 0.50071 |
| 10024 | I.S. | 0.50065 | 10025 | CARD11 | 0.50061 | 10026 | I.S. | 0.50058 |
| 10027 | I.S. | 0.50057 | 10028 | I.S. | 0.50056 | 10029 | HSD3B1 | 0.50053 |
| 10030 | I.S. | 0.50051 | 10031 | KRAS | 0.5005 | 10032 | TERT | 0.50049 |
| 10033 | I.S. | 0.50048 | 10034 | SETD2 | 0.50045 | 10035 | NOTCH1 | 0.50043 |
| 10036 | I.S. | 0.50041 | 10037 | I.S. | 0.5004 | 10038 | I.S. | 0.5004 |
| 10039 | I.S. | 0.50038 | 10040 | I.S. | 0.50033 | 10041 | I.S. | 0.50031 |
| 10042 | I.S. | 0.50028 | 10043 | PIK3CG | 0.50007 | 10044 | SBF2 | 0.50006 |
| 10045 | I.S. | 0.50005 | 10046 | PIK3R1 | 0.50005 | 10047 | I.S. | 0.50002 |
| 10048 | SMARCA4 | 0.50002 | 10049 | I.S. | 0.49998 | 10050 | I.S. | 0.49993 |
| 10051 | I.S. | 0.49985 | 10052 | ALK | 0.49978 | 10053 | SBF2 | 0.4997 |
| 10054 | I.S. | 0.49968 | 10055 | I.S. | 0.49967 | 10056 | KMT2C | 0.49966 |
| 10057 | CTC1 | 0.49965 | 10058 | I.S. | 0.49962 | 10059 | I.S. | 0.4996 |
| 10060 | I.S. | 0.4996 | 10061 | I.S. | 0.49954 | 10062 | WT1 | 0.49946 |
| 10063 | I.S. | 0.49941 | 10064 | SPTA1 | 0.49941 | 10065 | BCORL1 | 0.49936 |
| 10066 | HGF | 0.49934 | 10067 | SBF2 | 0.4993 | 10068 | I.S. | 0.49922 |
| 10069 | FAT1 | 0.49919 | 10070 | I.S. | 0.49919 | 10071 | I.S. | 0.49916 |
| 10072 | I.S. | 0.49907 | 10073 | CREBBP | 0.49898 | 10074 | APC | 0.49894 |
| 10075 | I.S. | 0.49887 | 10076 | I.S. | 0.49877 | 10077 | HGF | 0.49876 |
| 10078 | SMARCB1 | 0.49875 | 10079 | I.S. | 0.49875 | 10080 | I.S. | 0.49871 |
| 10081 | I.S. | 0.4987 | 10082 | I.S. | 0.49865 | 10083 | I.S. | 0.49858 |
| 10084 | I.S. | 0.49853 | 10085 | SBF2 | 0.49853 | 10086 | I.S. | 0.49851 |
| 10087 | I.S. | 0.4985 | 10088 | DKC1 | 0.49848 | 10089 | CUL3 | 0.49846 |
| 10090 | I.S. | 0.49842 | 10091 | KMT2D | 0.49835 | 10092 | AKT2 | 0.49832 |
| 10093 | RAD54L | 0.49816 | 10094 | I.S. | 0.49805 | 10095 | TP53 | 0.498 |
| 10096 | I.S. | 0.49793 | 10097 | I.S. | 0.49791 | 10098 | I.S. | 0.49788 |
| 10099 | ATRX | 0.49788 | 10100 | AXL | 0.49785 | 10101 | ANKRD26 | 0.49785 |
| 10102 | I.S. | 0.49785 | 10103 | ATG2B | 0.49773 | 10104 | FLCN | 0.49772 |
| 10105 | I.S. | 0.49771 | 10106 | TERF2 | 0.49768 | 10107 | I.S. | 0.49763 |
| 10108 | TCIRG1 | 0.49754 | 10109 | I.S. | 0.49754 | 10110 | I.S. | 0.49751 |
| 10111 | I.S. | 0.49747 | 10112 | TOP1 | 0.49746 | 10113 | FANCB | 0.49741 |
| 10114 | KDM5C | 0.49741 | 10115 | DNM2 | 0.49738 | 10116 | I.S. | 0.49729 |
| 10117 | I.S. | 0.49729 | 10118 | I.S. | 0.49727 | 10119 | I.S. | 0.49725 |
| 10120 | I.S. | 0.4971 | 10121 | I.S. | 0.49707 | 10122 | I.S. | 0.49705 |
| 10123 | I.S. | 0.49698 | 10124 | I.S. | 0.49694 | 10125 | I.S. | 0.49678 |
| 10126 | I.S. | 0.49677 | 10127 | I.S. | 0.49677 | 10128 | I.S. | 0.49664 |
| 10129 | I.S. | 0.49662 | 10130 | I.S. | 0.49648 | 10131 | I.S. | 0.49646 |
| 10132 | I.S. | 0.49645 | 10133 | CDAN1 | 0.49645 | 10134 | I.S. | 0.49642 |
| 10135 | I.S. | 0.49635 | 10136 | I.S. | 0.49628 | 10137 | FANCA | 0.49627 |
| 10138 | NTRK2 | 0.49613 | 10139 | I.S. | 0.49613 | 10140 | RUNX1T1 | 0.49613 |
| 10141 | I.S. | 0.49608 | 10142 | BRIP1 | 0.49608 | 10143 | I.S. | 0.49606 |
| 10144 | I.S. | 0.49601 | 10145 | SAMD9L | 0.49597 | 10146 | I.S. | 0.49587 |
| 10147 | KDR | 0.49583 | 10148 | I.S. | 0.49582 | 10149 | I.S. | 0.4958 |
| 10150 | I.S. | 0.49565 | 10151 | KAT6A | 0.49563 | 10152 | CREBBP | 0.49552 |
| 10153 | I.S. | 0.49546 | 10154 | I.S. | 0.49544 | 10155 | I.S. | 0.49529 |
| 10156 | I.S. | 0.49523 | 10157 | I.S. | 0.49516 | 10158 | I.S. | 0.49514 |
| 10159 | FANCM | 0.49514 | 10160 | I.S. | 0.49512 | 10161 | I.S. | 0.49512 |
| 10162 | I.S. | 0.49512 | 10163 | I.S. | 0.49512 | 10164 | I.S. | 0.49506 |
| 10165 | NSD1 | 0.49505 | 10166 | I.S. | 0.495 | 10167 | I.S. | 0.49492 |
| 10168 | I.S. | 0.49484 | 10169 | ERBB4 | 0.49482 | 10170 | I.S. | 0.49476 |
| 10171 | I.S. | 0.49465 | 10172 | I.S. | 0.49465 | 10173 | ASXL1 | 0.49465 |
| 10174 | I.S. | 0.49455 | 10175 | I.S. | 0.49449 | 10176 | I.S. | 0.49449 |
| 10177 | I.S. | 0.49447 | 10178 | AXIN1 | 0.49443 | 10179 | FGFR2 | 0.49438 |
| 10180 | I.S. | 0.49435 | 10181 | I.S. | 0.4943 | 10182 | I.S. | 0.49429 |
| 10183 | I.S. | 0.49428 | 10184 | I.S. | 0.4942 | 10185 | MTOR | 0.49419 |
| 10186 | I.S. | 0.49414 | 10187 | I.S. | 0.49413 | 10188 | TSC1 | 0.4941 |
| 10189 | I.S. | 0.4941 | 10190 | I.S. | 0.49407 | 10191 | PREX2 | 0.49405 |
| 10192 | I.S. | 0.49396 | 10193 | I.S. | 0.49384 | 10194 | AXL | 0.49382 |
| 10195 | NOTCH2 | 0.49381 | 10196 | BARD1 | 0.49381 | 10197 | I.S. | 0.49378 |
| 10198 | I.S. | 0.49378 | 10199 | I.S. | 0.49375 | 10200 | HGF | 0.49369 |
| 10201 | I.S. | 0.49364 | 10202 | I.S. | 0.49363 | 10203 | I.S. | 0.49361 |
| 10204 | I.S. | 0.49352 | 10205 | I.S. | 0.49349 | 10206 | MAP2K1 | 0.49348 |
| 10207 | I.S. | 0.49347 | 10208 | I.S. | 0.49345 | 10209 | IL7R | 0.49341 |
| 10210 | BRCA1 | 0.4934 | 10211 | I.S. | 0.49331 | 10212 | KMT2A | 0.49331 |
| 10213 | CDC73 | 0.49327 | 10214 | MSH6 | 0.49322 | 10215 | I.S. | 0.49308 |
| 10216 | I.S. | 0.49303 | 10217 | SAMD9L | 0.49303 | 10218 | KLF1 | 0.49298 |
| 10219 | EXO1 | 0.49298 | 10220 | I.S. | 0.49292 | 10221 | I.S. | 0.49278 |
| 10222 | NOTCH2 | 0.49268 | 10223 | SBF2 | 0.49262 | 10224 | I.S. | 0.49262 |
| 10225 | I.S. | 0.49262 | 10226 | I.S. | 0.49262 | 10227 | I.S. | 0.49261 |
| 10228 | I.S. | 0.4926 | 10229 | AR | 0.49249 | 10230 | MEN1 | 0.49247 |
| 10231 | TERF2IP | 0.49243 | 10232 | I.S. | 0.49243 | 10233 | I.S. | 0.49235 |
| 10234 | I.S. | 0.49234 | 10235 | I.S. | 0.49233 | 10236 | I.S. | 0.49229 |
| 10237 | I.S. | 0.49228 | 10238 | RANBP2 | 0.49224 | 10239 | GALNT12 | 0.49223 |
| 10240 | CTNNB1 | 0.49215 | 10241 | I.S. | 0.49214 | 10242 | DOT1L | 0.49213 |
| 10243 | I.S. | 0.49208 | 10244 | I.S. | 0.492 | 10245 | I.S. | 0.49199 |
| 10246 | I.S. | 0.49199 | 10247 | I.S. | 0.49196 | 10248 | ATR | 0.49185 |
| 10249 | MSH6 | 0.49182 | 10250 | I.S. | 0.49179 | 10251 | I.S. | 0.49178 |
| 10252 | I.S. | 0.49167 | 10253 | I.S. | 0.49167 | 10254 | I.S. | 0.49167 |
| 10255 | I.S. | 0.49166 | 10256 | PIK3CA | 0.49164 | 10257 | NOTCH3 | 0.49164 |
| 10258 | I.S. | 0.49159 | 10259 | I.S. | 0.49153 | 10260 | ANKRD26 | 0.49152 |
| 10261 | NBN | 0.49152 | 10262 | I.S. | 0.49149 | 10263 | I.S. | 0.49143 |
| 10264 | KMT2A | 0.49141 | 10265 | I.S. | 0.49137 | 10266 | AMER1 | 0.49134 |
| 10267 | I.S. | 0.49134 | 10268 | I.S. | 0.49131 | 10269 | KDM6A | 0.49129 |
| 10270 | GATA1 | 0.4912 | 10271 | I.S. | 0.49117 | 10272 | I.S. | 0.49114 |
| 10273 | I.S. | 0.49104 | 10274 | I.S. | 0.49104 | 10275 | I.S. | 0.49098 |
| 10276 | I.S. | 0.49098 | 10277 | I.S. | 0.49097 | 10278 | I.S. | 0.49095 |
| 10279 | ARAF | 0.49091 | 10280 | DKC1 | 0.4909 | 10281 | ARID1A | 0.49088 |
| 10282 | I.S. | 0.49087 | 10283 | I.S. | 0.49087 | 10284 | BRD4 | 0.49084 |
| 10285 | I.S. | 0.49083 | 10286 | I.S. | 0.49078 | 10287 | I.S. | 0.49078 |
| 10288 | I.S. | 0.49077 | 10289 | SF3B1 | 0.49074 | 10290 | ATR | 0.49071 |
| 10291 | FLT4 | 0.49068 | 10292 | I.S. | 0.49063 | 10293 | I.S. | 0.49062 |
| 10294 | I.S. | 0.4906 | 10295 | ABCB7 | 0.49058 | 10296 | PRKDC | 0.49057 |
| 10297 | I.S. | 0.49057 | 10298 | FANCD2 | 0.49057 | 10299 | ARID2 | 0.49057 |
| 10300 | I.S. | 0.49052 | 10301 | I.S. | 0.49048 | 10302 | I.S. | 0.49045 |
| 10303 | MVK | 0.49039 | 10304 | KMT2C | 0.49037 | 10305 | I.S. | 0.49036 |
| 10306 | TERF2 | 0.49032 | 10307 | I.S. | 0.4903 | 10308 | I.S. | 0.49025 |
| 10309 | I.S. | 0.49022 | 10310 | TSC2 | 0.49022 | 10311 | NOTCH1 | 0.49021 |
| 10312 | I.S. | 0.49019 | 10313 | I.S. | 0.49018 | 10314 | AKT1 | 0.49011 |
| 10315 | I.S. | 0.4901 | 10316 | I.S. | 0.4901 | 10317 | I.S. | 0.49009 |
| 10318 | I.S. | 0.49004 | 10319 | I.S. | 0.49001 | 10320 | I.S. | 0.48994 |
| 10321 | I.S. | 0.48992 | 10322 | I.S. | 0.48989 | 10323 | I.S. | 0.4898 |
| 10324 | I.S. | 0.48977 | 10325 | I.S. | 0.48964 | 10326 | FGFR1 | 0.48959 |
| 10327 | I.S. | 0.48959 | 10328 | I.S. | 0.48956 | 10329 | I.S. | 0.48953 |
| 10330 | FGF19 | 0.48952 | 10331 | I.S. | 0.48947 | 10332 | RPTOR | 0.48939 |
| 10333 | PPM1D | 0.48936 | 10334 | I.S. | 0.48933 | 10335 | I.S. | 0.4893 |
| 10336 | I.S. | 0.48929 | 10337 | I.S. | 0.48926 | 10338 | I.S. | 0.48926 |
| 10339 | I.S. | 0.48923 | 10340 | CDAN1 | 0.48923 | 10341 | I.S. | 0.48919 |
| 10342 | CHD4 | 0.48913 | 10343 | I.S. | 0.48912 | 10344 | MDM2 | 0.48911 |
| 10345 | JAK3 | 0.48905 | 10346 | I.S. | 0.48883 | 10347 | I.S. | 0.48878 |
| 10348 | MYCN | 0.48873 | 10349 | RAD51D | 0.48871 | 10350 | I.S. | 0.48867 |
| 10351 | NBN | 0.48867 | 10352 | I.S. | 0.48858 | 10353 | I.S. | 0.48854 |
| 10354 | NLRP3 | 0.48854 | 10355 | PRKDC | 0.48853 | 10356 | I.S. | 0.48852 |
| 10357 | I.S. | 0.48852 | 10358 | KMT2C | 0.48851 | 10359 | I.S. | 0.48849 |
| 10360 | I.S. | 0.48846 | 10361 | I.S. | 0.48844 | 10362 | I.S. | 0.48843 |
| 10363 | I.S. | 0.48843 | 10364 | ASXL1 | 0.48843 | 10365 | I.S. | 0.48832 |
| 10366 | ERRFI1 | 0.48831 | 10367 | I.S. | 0.48828 | 10368 | FANCD2 | 0.48825 |
| 10369 | DICER1 | 0.4882 | 10370 | I.S. | 0.4882 | 10371 | GATA1 | 0.4882 |
| 10372 | I.S. | 0.4882 | 10373 | BCOR | 0.48817 | 10374 | I.S. | 0.48808 |
| 10375 | I.S. | 0.48805 | 10376 | DNM2 | 0.48804 | 10377 | MET | 0.48793 |
| 10378 | I.S. | 0.48792 | 10379 | IL7R | 0.48791 | 10380 | I.S. | 0.4879 |
| 10381 | I.S. | 0.48786 | 10382 | I.S. | 0.4878 | 10383 | I.S. | 0.4878 |
| 10384 | FOXL2 | 0.48777 | 10385 | I.S. | 0.48775 | 10386 | I.S. | 0.48772 |
| 10387 | I.S. | 0.48769 | 10388 | EMSY | 0.48769 | 10389 | EPAS1 | 0.48757 |
| 10390 | I.S. | 0.48757 | 10391 | ERBB3 | 0.48752 | 10392 | I.S. | 0.48751 |
| 10393 | I.S. | 0.48748 | 10394 | GRIN2A | 0.48746 | 10395 | WAS | 0.48737 |
| 10396 | I.S. | 0.48736 | 10397 | I.S. | 0.4873 | 10398 | I.S. | 0.4873 |
| 10399 | KMT2C | 0.48728 | 10400 | I.S. | 0.48726 | 10401 | I.S. | 0.48726 |
| 10402 | I.S. | 0.48721 | 10403 | I.S. | 0.48719 | 10404 | DNM2 | 0.48718 |
| 10405 | I.S. | 0.48715 | 10406 | I.S. | 0.48711 | 10407 | KLHL6 | 0.4871 |
| 10408 | I.S. | 0.48706 | 10409 | I.S. | 0.48697 | 10410 | I.S. | 0.48695 |
| 10411 | I.S. | 0.48693 | 10412 | I.S. | 0.48692 | 10413 | PRKDC | 0.48685 |
| 10414 | XPO1 | 0.48677 | 10415 | ASXL1 | 0.48677 | 10416 | PIK3CG | 0.48674 |
| 10417 | FANCD2 | 0.48674 | 10418 | KEAP1 | 0.48671 | 10419 | I.S. | 0.48671 |
| 10420 | I.S. | 0.48665 | 10421 | RICTOR | 0.48662 | 10422 | I.S. | 0.48662 |
| 10423 | I.S. | 0.4866 | 10424 | I.S. | 0.48659 | 10425 | I.S. | 0.48658 |
| 10426 | I.S. | 0.48656 | 10427 | FANCC | 0.48656 | 10428 | I.S. | 0.4865 |
| 10429 | BCOR | 0.48644 | 10430 | I.S. | 0.48642 | 10431 | I.S. | 0.4863 |
| 10432 | MRE11 | 0.48629 | 10433 | I.S. | 0.48628 | 10434 | MSH2 | 0.48623 |
| 10435 | I.S. | 0.48619 | 10436 | I.S. | 0.48612 | 10437 | GALNT12 | 0.48612 |
| 10438 | I.S. | 0.48611 | 10439 | I.S. | 0.48611 | 10440 | CUX1 | 0.48606 |
| 10441 | I.S. | 0.48605 | 10442 | CHD4 | 0.48603 | 10443 | I.S. | 0.48586 |
| 10444 | I.S. | 0.48579 | 10445 | I.S. | 0.48578 | 10446 | I.S. | 0.48567 |
| 10447 | I.S. | 0.48564 | 10448 | I.S. | 0.48561 | 10449 | WT1 | 0.4856 |
| 10450 | I.S. | 0.4855 | 10451 | I.S. | 0.4854 | 10452 | KMT2C | 0.4853 |
| 10453 | ERBB4 | 0.48529 | 10454 | I.S. | 0.48508 | 10455 | I.S. | 0.48504 |
| 10456 | WAS | 0.48502 | 10457 | I.S. | 0.48498 | 10458 | KDM5C | 0.48496 |
| 10459 | ZNF703 | 0.48496 | 10460 | I.S. | 0.4849 | 10461 | I.S. | 0.48488 |
| 10462 | CDK4 | 0.48487 | 10463 | PTPN11 | 0.48483 | 10464 | I.S. | 0.48481 |
| 10465 | PIK3CG | 0.4848 | 10466 | I.S. | 0.4848 | 10467 | KMT2A | 0.4848 |
| 10468 | I.S. | 0.48477 | 10469 | I.S. | 0.48472 | 10470 | I.S. | 0.48472 |
| 10471 | FLCN | 0.48469 | 10472 | I.S. | 0.48464 | 10473 | RICTOR | 0.48459 |
| 10474 | BMPR1A | 0.48457 | 10475 | I.S. | 0.48451 | 10476 | I.S. | 0.48451 |
| 10477 | FANCB | 0.48437 | 10478 | I.S. | 0.48431 | 10479 | IKZF1 | 0.48426 |
| 10480 | KRAS | 0.48425 | 10481 | PTPN11 | 0.48424 | 10482 | I.S. | 0.48419 |
| 10483 | RUNX1 | 0.48419 | 10484 | I.S. | 0.48418 | 10485 | CREBBP | 0.48415 |
| 10486 | I.S. | 0.48406 | 10487 | I.S. | 0.48398 | 10488 | I.S. | 0.48395 |
| 10489 | I.S. | 0.48384 | 10490 | I.S. | 0.48384 | 10491 | I.S. | 0.4837 |
| 10492 | I.S. | 0.48367 | 10493 | I.S. | 0.48361 | 10494 | I.S. | 0.48341 |
| 10495 | I.S. | 0.48338 | 10496 | I.S. | 0.4833 | 10497 | I.S. | 0.4833 |
| 10498 | MSH6 | 0.48323 | 10499 | ATM | 0.48323 | 10500 | CHD4 | 0.48321 |
| 10501 | JAK3 | 0.48319 | 10502 | I.S. | 0.48312 | 10503 | AXIN2 | 0.48307 |
| 10504 | I.S. | 0.48302 | 10505 | VHL | 0.483 | 10506 | I.S. | 0.48299 |
| 10507 | I.S. | 0.48294 | 10508 | I.S. | 0.48291 | 10509 | CREBBP | 0.48288 |
| 10510 | RANBP2 | 0.48278 | 10511 | EPAS1 | 0.48267 | 10512 | NSD1 | 0.48267 |
| 10513 | I.S. | 0.48266 | 10514 | I.S. | 0.48266 | 10515 | HGF | 0.48263 |
| 10516 | I.S. | 0.48262 | 10517 | ABCB7 | 0.48259 | 10518 | I.S. | 0.48258 |
| 10519 | SMARCB1 | 0.48258 | 10520 | I.S. | 0.48257 | 10521 | ROS1 | 0.48257 |
| 10522 | RTEL1 | 0.48255 | 10523 | I.S. | 0.48255 | 10524 | I.S. | 0.48255 |
| 10525 | I.S. | 0.48255 | 10526 | I.S. | 0.48252 | 10527 | I.S. | 0.48238 |
| 10528 | TERT | 0.48237 | 10529 | MTOR | 0.48235 | 10530 | I.S. | 0.48233 |
| 10531 | I.S. | 0.48224 | 10532 | EMSY | 0.48216 | 10533 | RBBP6 | 0.48205 |
| 10534 | I.S. | 0.48203 | 10535 | I.S. | 0.48202 | 10536 | ADGRA2 | 0.4819 |
| 10537 | I.S. | 0.48189 | 10538 | I.S. | 0.48189 | 10539 | ANKRD26 | 0.48184 |
| 10540 | I.S. | 0.48184 | 10541 | TCIRG1 | 0.48177 | 10542 | CDK12 | 0.48175 |
| 10543 | I.S. | 0.48174 | 10544 | I.S. | 0.48172 | 10545 | I.S. | 0.48172 |
| 10546 | I.S. | 0.48171 | 10547 | I.S. | 0.4817 | 10548 | I.S. | 0.4817 |
| 10549 | FANCI | 0.48166 | 10550 | I.S. | 0.48163 | 10551 | ARID2 | 0.48163 |
| 10552 | ZRSR2 | 0.48163 | 10553 | I.S. | 0.48162 | 10554 | TRAF3 | 0.48157 |
| 10555 | RANBP2 | 0.48151 | 10556 | I.S. | 0.48141 | 10557 | I.S. | 0.4814 |
| 10558 | I.S. | 0.48137 | 10559 | I.S. | 0.48125 | 10560 | RAD51D | 0.48125 |
| 10561 | I.S. | 0.48119 | 10562 | I.S. | 0.48119 | 10563 | ATRX | 0.48116 |
| 10564 | I.S. | 0.48107 | 10565 | I.S. | 0.48106 | 10566 | I.S. | 0.48105 |
| 10567 | I.S. | 0.48098 | 10568 | CREBBP | 0.48098 | 10569 | I.S. | 0.48097 |
| 10570 | SMARCA4 | 0.48096 | 10571 | KDM6A | 0.48096 | 10572 | ERRFI1 | 0.48094 |
| 10573 | I.S. | 0.4809 | 10574 | I.S. | 0.48087 | 10575 | MED12 | 0.48083 |
| 10576 | LRP1B | 0.48075 | 10577 | KMT2D | 0.4807 | 10578 | I.S. | 0.48069 |
| 10579 | JAK2 | 0.48069 | 10580 | PDGFRA | 0.48065 | 10581 | DDX41 | 0.48062 |
| 10582 | KDM6A | 0.4805 | 10583 | I.S. | 0.48047 | 10584 | MSH2 | 0.48044 |
| 10585 | TSHR | 0.48044 | 10586 | AR | 0.48042 | 10587 | I.S. | 0.48038 |
| 10588 | BRCA2 | 0.48038 | 10589 | I.S. | 0.48038 | 10590 | I.S. | 0.48036 |
| 10591 | I.S. | 0.48028 | 10592 | LYST | 0.48025 | 10593 | PTPN11 | 0.48023 |
| 10594 | I.S. | 0.48022 | 10595 | I.S. | 0.48015 | 10596 | I.S. | 0.48014 |
| 10597 | I.S. | 0.48008 | 10598 | I.S. | 0.48004 | 10599 | CDAN1 | 0.48002 |
| 10600 | I.S. | 0.48 | 10601 | I.S. | 0.47985 | 10602 | I.S. | 0.47983 |
| 10603 | BAP1 | 0.47983 | 10604 | I.S. | 0.47982 | 10605 | DKC1 | 0.47979 |
| 10606 | I.S. | 0.4797 | 10607 | BRAF | 0.47967 | 10608 | I.S. | 0.47963 |
| 10609 | I.S. | 0.47963 | 10610 | EPCAM | 0.47961 | 10611 | BCOR | 0.47959 |
| 10612 | AURKA | 0.47959 | 10613 | ALK | 0.47952 | 10614 | TSC2 | 0.47952 |
| 10615 | I.S. | 0.47951 | 10616 | KMT2B | 0.47948 | 10617 | KDM6A | 0.47947 |
| | BCYRN1, | 0.4794 | 10619 | NOTCH2 | 0.4792 | 10620 | I.S. | 0.47919 |
| | TAF1 | | | | | | | |
| 10621 | NOTCH2 | 0.47913 | 10622 | PIK3R2 | 0.4791 | 10623 | I.S. | 0.47907 |
| 10624 | I.S. | 0.47904 | 10625 | GRM3 | 0.47899 | 10626 | I.S. | 0.47899 |
| 10627 | PRKDC | 0.47895 | 10628 | NF1 | 0.47893 | 10629 | I.S. | 0.47887 |
| 10630 | I.S. | 0.47885 | 10631 | PMS1 | 0.47872 | 10632 | IGF1R | 0.47871 |
| 10633 | EPAS1 | 0.47869 | 10634 | DKC1 | 0.47864 | 10635 | I.S. | 0.47863 |
| 10636 | I.S. | 0.47858 | 10637 | SUZ12 | 0.47857 | 10638 | GNAS | 0.47854 |
| 10639 | I.S. | 0.47851 | 10640 | I.S. | 0.47845 | 10641 | I.S. | 0.47837 |
| 10642 | I.S. | 0.47836 | 10643 | I.S. | 0.47827 | 10644 | I.S. | 0.47824 |
| 10645 | BCOR | 0.47821 | 10646 | BCYRN1, | 0.47821 | 10647 | SAMD9L | 0.47819 |
| | | | | TAF1 | | | | |
| 10648 | I.S. | 0.47812 | 10649 | I.S. | 0.47812 | 10650 | I.S. | 0.47809 |
| 10651 | GRIN2A | 0.47807 | 10652 | I.S. | 0.47807 | 10653 | RET | 0.47798 |
| 10654 | MSH6 | 0.47789 | 10655 | PIK3CB | 0.47789 | 10656 | CREBBP | 0.47786 |
| 10657 | FBXW7 | 0.47783 | 10658 | POLE | 0.4778 | 10659 | I.S. | 0.47775 |
| 10660 | FOXP1 | 0.47773 | 10661 | I.S. | 0.47768 | 10662 | I.S. | 0.47767 |
| 10663 | I.S. | 0.47764 | 10664 | CDAN1 | 0.47761 | 10665 | SAMD9L | 0.4776 |
| 10666 | SMARCA4 | 0.47759 | 10667 | KDR | 0.47759 | 10668 | I.S. | 0.47759 |
| 10669 | ATG2B | 0.47758 | 10670 | I.S. | 0.47756 | 10671 | CTC1 | 0.47756 |
| 10672 | DKC1 | 0.47754 | 10673 | SF3B1 | 0.47754 | 10674 | I.S. | 0.4775 |
| 10675 | I.S. | 0.47748 | 10676 | CBLB | 0.47747 | 10677 | ATRX | 0.47744 |
| 10678 | I.S. | 0.47744 | 10679 | I.S. | 0.47738 | 10680 | PBRM1 | 0.47735 |
| 10681 | I.S. | 0.47735 | 10682 | I.S. | 0.47733 | 10683 | I.S. | 0.47729 |
| 10684 | I.S. | 0.47728 | 10685 | LRP1B | 0.47727 | 10686 | GATA6 | 0.47727 |
| 10687 | I.S. | 0.47718 | 10688 | I.S. | 0.47715 | 10689 | I.S. | 0.47714 |
| 10690 | I.S. | 0.47714 | 10691 | I.S. | 0.47713 | 10692 | KDR | 0.47707 |
| 10693 | SPTA1 | 0.47705 | 10694 | I.S. | 0.47703 | 10695 | I.S. | 0.47693 |
| 10696 | KMT2C | 0.47684 | 10697 | I.S. | 0.47681 | 10698 | ARID2 | 0.47681 |
| 10699 | RANBP2 | 0.47678 | 10700 | KMT2D | 0.47676 | 10701 | I.S. | 0.47672 |
| 10702 | PRKAR1A | 0.4767 | 10703 | I.S. | 0.47667 | 10704 | I.S. | 0.47666 |
| 10705 | TERT | 0.47664 | 10706 | I.S. | 0.47663 | 10707 | I.S. | 0.47656 |
| 10708 | I.S. | 0.47653 | 10709 | BMPR1A | 0.4765 | 10710 | I.S. | 0.47637 |
| 10711 | STAT4 | 0.47637 | 10712 | EMSY | 0.47634 | 10713 | I.S. | 0.47634 |
| 10714 | I.S. | 0.47631 | 10715 | I.S. | 0.47631 | 10716 | I.S. | 0.47631 |
| 10717 | SMARCA4 | 0.47631 | 10718 | I.S. | 0.47628 | 10719 | I.S. | 0.47625 |
| 10720 | MSH6 | 0.47625 | 10721 | SMC1A | 0.47617 | 10722 | RAC1 | 0.47609 |
| 10723 | I.S. | 0.47607 | 10724 | I.S. | 0.47604 | 10725 | I.S. | 0.47601 |
| 10726 | I.S. | 0.47598 | 10727 | DKC1 | 0.47596 | 10728 | CDAN1 | 0.47593 |
| 10729 | I.S. | 0.4759 | 10730 | SBF2 | 0.47584 | 10731 | I.S. | 0.47581 |
| 10732 | I.S. | 0.4758 | 10733 | BRCA1 | 0.47574 | 10734 | EPHB1 | 0.47571 |
| 10735 | KMT2A | 0.47568 | 10736 | I.S. | 0.47566 | 10737 | I.S. | 0.47563 |
| 10738 | I.S. | 0.47559 | 10739 | I.S. | 0.47555 | 10740 | I.S. | 0.47553 |
| 10741 | I.S. | 0.47544 | 10742 | ANKRD26 | 0.47543 | 10743 | I.S. | 0.47533 |
| 10744 | I.S. | 0.47527 | 10745 | ATG2B | 0.47527 | 10746 | EED | 0.47525 |
| 10747 | I.S. | 0.47525 | 10748 | FAT1 | 0.47519 | 10749 | GNAS | 0.47518 |
| 10750 | I.S. | 0.47516 | 10751 | I.S. | 0.47516 | 10752 | I.S. | 0.47516 |
| 10753 | I.S. | 0.47516 | 10754 | PRKDC | 0.47513 | 10755 | I.S. | 0.47512 |
| 10756 | — | 0.47512 | 10757 | AKT1 | 0.47505 | 10758 | I.S. | 0.47503 |
| 10759 | I.S. | 0.47501 | 10760 | I.S. | 0.475 | 10761 | TET2 | 0.47497 |
| 10762 | I.S. | 0.47492 | 10763 | I.S. | 0.47492 | 10764 | I.S. | 0.47488 |
| 10765 | I.S. | 0.47485 | 10766 | I.S. | 0.47485 | 10767 | PMS2 | 0.47481 |
| 10768 | I.S. | 0.4748 | 10769 | — | 0.47476 | 10770 | NOTCH3 | 0.47473 |
| 10771 | I.S. | 0.47469 | 10772 | FGFR3 | 0.47467 | 10773 | I.S. | 0.47466 |
| 10774 | NOTCH1 | 0.47462 | 10775 | I.S. | 0.47459 | 10776 | I.S. | 0.47452 |
| 10777 | EXO1 | 0.4745 | 10778 | I.S. | 0.4745 | 10779 | I.S. | 0.47449 |
| 10780 | POT1 | 0.47448 | 10781 | SDHC | 0.47442 | 10782 | I.S. | 0.47442 |
| 10783 | ATR | 0.47441 | 10784 | ATM | 0.47434 | 10785 | I.S. | 0.47432 |
| 10786 | I.S. | 0.47429 | 10787 | I.S. | 0.47422 | 10788 | NOTCH3 | 0.47421 |
| 10789 | I.S. | 0.47413 | 10790 | I.S. | 0.47408 | 10791 | I.S. | 0.47406 |
| 10792 | I.S. | 0.47404 | 10793 | KDM6A | 0.47403 | 10794 | I.S. | 0.47401 |
| 10795 | I.S. | 0.47392 | 10796 | MUTYH | 0.47381 | 10797 | I.S. | 0.4738 |
| 10798 | I.S. | 0.47379 | 10799 | I.S. | 0.47373 | 10800 | I.S. | 0.47373 |
| 10801 | I.S. | 0.47372 | 10802 | I.S. | 0.4737 | 10803 | I.S. | 0.47365 |
| 10804 | FGFR1 | 0.47363 | 10805 | I.S. | 0.4736 | 10806 | I.S. | 0.47357 |
| 10807 | NSD1 | 0.47352 | 10808 | SPOP | 0.47349 | 10809 | I.S. | 0.47348 |
| 10810 | RANBP2 | 0.47346 | 10811 | I.S. | 0.47343 | 10812 | I.S. | 0.47341 |
| 10813 | I.S. | 0.47336 | 10814 | I.S. | 0.47327 | 10815 | I.S. | 0.47325 |
| 10816 | I.S. | 0.47319 | 10817 | I.S. | 0.47319 | 10818 | FANCD2 | 0.47314 |
| 10819 | RBM10 | 0.47305 | 10820 | I.S. | 0.47302 | 10821 | I.S. | 0.47302 |
| 10822 | I.S. | 0.47301 | 10823 | GNAQ | 0.47298 | 10824 | I.S. | 0.47298 |
| 10825 | I.S. | 0.47289 | 10826 | SBDS | 0.47289 | 10827 | I.S. | 0.47287 |
| 10828 | ATR | 0.47286 | 10829 | MAGI2 | 0.47284 | 10830 | TET2 | 0.47283 |
| 10831 | MSH2 | 0.47281 | 10832 | PIK3R1 | 0.47281 | 10833 | CALR | 0.47281 |
| 10834 | SUZ12 | 0.47275 | 10835 | I.S. | 0.47269 | 10836 | FANCD2 | 0.47265 |
| 10837 | I.S. | 0.4726 | 10838 | IGF2 | 0.47259 | 10839 | I.S. | 0.47252 |
| 10840 | I.S. | 0.47245 | 10841 | CDK6 | 0.47244 | 10842 | GRIN2A | 0.47241 |
| 10843 | ATRX | 0.47239 | 10844 | TSC1 | 0.47237 | 10845 | MAP2K1 | 0.47237 |
| 10846 | I.S. | 0.4723 | 10847 | I.S. | 0.47229 | 10848 | MAGI2 | 0.47222 |
| 10849 | I.S. | 0.47221 | 10850 | I.S. | 0.47221 | 10851 | TCF3 | 0.47221 |
| 10852 | I.S. | 0.47215 | 10853 | ATR | 0.47212 | 10854 | CHD4 | 0.47208 |
| 10855 | ATRX | 0.47207 | 10856 | I.S. | 0.472 | 10857 | I.S. | 0.47199 |
| 10858 | RB1 | 0.47198 | 10859 | I.S. | 0.47195 | 10860 | KEL | 0.47195 |
| 10861 | I.S. | 0.47194 | 10862 | SPEN | 0.47183 | 10863 | I.S. | 0.47182 |
| 10864 | ATRX | 0.47177 | 10865 | RAD54L | 0.47174 | 10866 | ALK | 0.47174 |
| 10867 | I.S. | 0.47174 | 10868 | I.S. | 0.47173 | 10869 | I.S. | 0.47168 |
| 10870 | ARAF | 0.47165 | 10871 | I.S. | 0.47162 | 10872 | I.S. | 0.4716 |
| 10873 | CDK12 | 0.47155 | 10874 | I.S. | 0.47153 | 10875 | I.S. | 0.47147 |
| 10876 | CUL3 | 0.47146 | 10877 | I.S. | 0.47143 | 10878 | I.S. | 0.47138 |
| 10879 | BTK | 0.47136 | 10880 | I.S. | 0.47132 | 10881 | I.S. | 0.47132 |
| 10882 | I.S. | 0.47127 | 10883 | SPOP | 0.47127 | 10884 | CHD2 | 0.47121 |
| 10885 | APC | 0.47121 | 10886 | I.S. | 0.4712 | 10887 | CREBBP | 0.47119 |
| 10888 | I.S. | 0.47115 | 10889 | ATG2B | 0.47114 | 10890 | PAK3 | 0.47112 |
| 10891 | LRP1B | 0.47105 | 10892 | I.S. | 0.47102 | 10893 | I.S. | 0.47102 |
| 10894 | RICTOR | 0.47102 | 10895 | BCOR | 0.47097 | 10896 | SAMD9L | 0.47097 |
| 10897 | I.S. | 0.47088 | 10898 | KMT2D | 0.47087 | 10899 | I.S. | 0.47079 |
| 10900 | I.S. | 0.47078 | 10901 | I.S. | 0.47076 | 10902 | ABL2 | 0.47072 |
| 10903 | I.S. | 0.47071 | 10904 | ARAF | 0.4707 | 10905 | I.S. | 0.47058 |
| 10906 | I.S. | 0.47056 | 10907 | I.S. | 0.47047 | 10908 | I.S. | 0.47046 |
| 10909 | MET | 0.47043 | 10910 | I.S. | 0.4704 | 10911 | PAK3 | 0.47035 |
| 10912 | PTPN11 | 0.47031 | 10913 | PLCG2 | 0.47029 | 10914 | RBBP6 | 0.47028 |
| 10915 | I.S. | 0.47028 | 10916 | I.S. | 0.47023 | 10917 | I.S. | 0.47022 |
| 10918 | I.S. | 0.47019 | 10919 | PIK3CG | 0.47011 | 10920 | CHD2 | 0.4701 |
| 10921 | I.S. | 0.47007 | 10922 | PIK3CB | 0.47002 | 10923 | I.S. | 0.47002 |
| 10924 | I.S. | 0.47 | 10925 | PIK3R1 | 0.46994 | 10926 | PLCG1 | 0.4699 |
| 10927 | ERBB3 | 0.46987 | 10928 | I.S. | 0.46987 | 10929 | I.S. | 0.46981 |
| 10930 | I.S. | 0.4698 | 10931 | I.S. | 0.46979 | 10932 | FUBP1 | 0.46978 |
| 10933 | I.S. | 0.46975 | 10934 | SLX4 | 0.46974 | 10935 | I.S. | 0.46972 |
| 10936 | I.S. | 0.4697 | 10937 | I.S. | 0.46968 | 10938 | BCORL1 | 0.46967 |
| 10939 | SMC1A | 0.46966 | 10940 | I.S. | 0.46964 | 10941 | I.S. | 0.46963 |
| 10942 | TET2 | 0.46961 | 10943 | BCR | 0.4696 | 10944 | I.S. | 0.46959 |
| 10945 | I.S. | 0.46959 | 10946 | I.S. | 0.46956 | 10947 | I.S. | 0.46951 |
| 10948 | I.S. | 0.46947 | 10949 | DKC1 | 0.46943 | 10950 | I.S. | 0.46942 |
| 10951 | I.S. | 0.46939 | 10952 | I.S. | 0.46939 | 10953 | GATA1 | 0.46934 |
| 10954 | I.S. | 0.46933 | 10955 | BRAF | 0.46933 | 10956 | I.S. | 0.4693 |
| 10957 | I.S. | 0.46924 | 10958 | I.S. | 0.46921 | 10959 | I.S. | 0.46921 |
| 10960 | I.S. | 0.46918 | 10961 | I.S. | 0.46917 | 10962 | I.S. | 0.46917 |
| 10963 | I.S. | 0.46912 | 10964 | I.S. | 0.46912 | 10965 | TERF1 | 0.4691 |
| 10966 | KMT2D | 0.46909 | 10967 | I.S. | 0.46904 | 10968 | NPM1 | 0.469 |
| 10969 | I.S. | 0.469 | 10970 | NOTCH2 | 0.46898 | 10971 | CUL3 | 0.46895 |
| 10972 | I.S. | 0.46892 | 10973 | I.S. | 0.4689 | 10974 | I.S. | 0.46885 |
| 10975 | I.S. | 0.46883 | 10976 | I.S. | 0.46883 | 10977 | I.S. | 0.46877 |
| 10978 | I.S. | 0.46875 | 10979 | I.S. | 0.46871 | 10980 | ARAF | 0.46868 |
| 10981 | I.S. | 0.46867 | 10982 | I.S. | 0.46865 | 10983 | I.S. | 0.46864 |
| 10984 | SLX4 | 0.46864 | 10985 | KMT2D | 0.46856 | 10986 | BCYRN1, | 0.46856 |
| | | | | | | | TAF1 | |
| 10987 | I.S. | 0.4685 | 10988 | I.S. | 0.46849 | 10989 | NOTCH2 | 0.46842 |
| 10990 | I.S. | 0.46835 | 10991 | ABCB7 | 0.46827 | 10992 | I.S. | 0.46826 |
| 10993 | TERT | 0.46825 | 10994 | I.S. | 0.46824 | 10995 | I.S. | 0.46818 |
| 10996 | FLCN | 0.46817 | 10997 | RICTOR | 0.46817 | 10998 | I.S. | 0.46809 |
| 10999 | SLIT2 | 0.46809 | 11000 | PLCG2 | 0.46808 | 11001 | I.S. | 0.46808 |
| 11002 | NTRK2 | 0.46791 | 11003 | I.S. | 0.46791 | 11004 | I.S. | 0.46789 |
| 11005 | I.S. | 0.46788 | 11006 | I.S. | 0.46786 | 11007 | CREBBP | 0.46785 |
| 11008 | I.S. | 0.46785 | 11009 | I.S. | 0.46785 | 11010 | I.S. | 0.4678 |
| 11011 | I.S. | 0.46776 | 11012 | I.S. | 0.46773 | 11013 | I.S. | 0.46761 |
| 11014 | I.S. | 0.46757 | 11015 | I.S. | 0.46748 | 11016 | SAMD9L | 0.46747 |
| 11017 | BCYRN1, | 0.46741 | 11018 | I.S. | 0.4674 | 11019 | I.S. | 0.46736 |
| | TAF1 | | | | | | | |
| 11020 | I.S. | 0.4673 | 11021 | ROS1 | 0.46728 | 11022 | I.S. | 0.46728 |
| 11023 | STAG2 | 0.46726 | 11024 | I.S. | 0.46726 | 11025 | I.S. | 0.46719 |
| 11026 | SMARCA4 | 0.46717 | 11027 | I.S. | 0.46715 | 11028 | I.S. | 0.46713 |
| 11029 | I.S. | 0.46707 | 11030 | I.S. | 0.46706 | 11031 | I.S. | 0.46698 |
| 11032 | I.S. | 0.46694 | 11033 | DNM2 | 0.46694 | 11034 | STAG2 | 0.4669 |
| 11035 | I.S. | 0.4669 | 11036 | SMARCA4 | 0.4669 | 11037 | I.S. | 0.46688 |
| 11038 | MTOR | 0.46687 | 11039 | ATRX | 0.46684 | 11040 | I.S. | 0.46681 |
| 11041 | I.S. | 0.4668 | 11042 | I.S. | 0.46677 | 11043 | FANCA | 0.46669 |
| 11044 | AMER1 | 0.46668 | 11045 | GRM3 | 0.46667 | 11046 | SRC | 0.46665 |
| 11047 | I.S. | 0.46662 | 11048 | I.S. | 0.4666 | 11049 | I.S. | 0.46657 |
| 11050 | I.S. | 0.46655 | 11051 | I.S. | 0.46651 | 11052 | CHD2 | 0.46651 |
| 11053 | PTCH1 | 0.46645 | 11054 | I.S. | 0.46641 | 11055 | RBBP6 | 0.46637 |
| 11056 | I.S. | 0.46633 | 11057 | NFE2L2 | 0.46633 | 11058 | I.S. | 0.46632 |
| 11059 | I.S. | 0.4662 | 11060 | I.S. | 0.46613 | 11061 | FANCE | 0.46612 |
| 11062 | SLIT2 | 0.4661 | 11063 | NTRK1 | 0.46609 | 11064 | PLCG1 | 0.46599 |
| 11065 | I.S. | 0.46597 | 11066 | I.S. | 0.46596 | 11067 | I.S. | 0.46593 |
| 11068 | SPEN | 0.46592 | 11069 | I.S. | 0.46591 | 11070 | I.S. | 0.46588 |
| 11071 | KMT2C | 0.46574 | 11072 | EMSY | 0.46572 | 11073 | JAGN1 | 0.46568 |
| 11074 | I.S. | 0.46566 | 11075 | I.S. | 0.46561 | 11076 | I.S. | 0.4655 |
| 11077 | I.S. | 0.46544 | 11078 | I.S. | 0.46539 | 11079 | RNF43 | 0.46523 |
| 11080 | EPHA5 | 0.46523 | 11081 | I.S. | 0.46518 | 11082 | I.S. | 0.46517 |
| 11083 | I.S. | 0.46508 | 11084 | — | 0.46506 | 11085 | BCR | 0.46502 |
| 11086 | I.S. | 0.46501 | 11087 | I.S. | 0.46499 | 11088 | ANKRD26 | 0.46496 |
| 11089 | I.S. | 0.46494 | 11090 | I.S. | 0.46492 | 11091 | I.S. | 0.46476 |
| 11092 | I.S. | 0.46473 | 11093 | I.S. | 0.46471 | 11094 | EPAS1 | 0.46471 |
| 11095 | I.S. | 0.46469 | 11096 | SBF2 | 0.46463 | 11097 | I.S. | 0.46461 |
| 11098 | I.S. | 0.46457 | 11099 | EGFR | 0.46457 | 11100 | I.S. | 0.46451 |
| 11101 | RBM10 | 0.46449 | 11102 | I.S. | 0.46446 | 11103 | I.S. | 0.46446 |
| 11104 | BRD4 | 0.46445 | 11105 | I.S. | 0.46437 | 11106 | I.S. | 0.46436 |
| 11107 | I.S. | 0.46435 | 11108 | I.S. | 0.46425 | 11109 | SMARCA4 | 0.46425 |
| 11110 | MUTYH | 0.46416 | 11111 | I.S. | 0.46413 | 11112 | I.S. | 0.4641 |
| 11113 | I.S. | 0.4641 | 11114 | I.S. | 0.46408 | 11115 | SPEN | 0.46404 |
| 11116 | I.S. | 0.46403 | 11117 | I.S. | 0.46397 | 11118 | I.S. | 0.46397 |
| 11119 | PMS1 | 0.46394 | 11120 | I.S. | 0.46393 | 11121 | KMT2D | 0.46393 |
| 11122 | NOTCH1 | 0.46392 | 11123 | EGLN1 | 0.46377 | 11124 | I.S. | 0.46377 |
| 11125 | I.S. | 0.46374 | 11126 | I.S. | 0.46372 | 11127 | I.S. | 0.46369 |
| 11128 | I.S. | 0.46366 | 11129 | CREBBP | 0.46365 | 11130 | I.S. | 0.46363 |
| 11131 | I.S. | 0.4636 | 11132 | I.S. | 0.46359 | 11133 | MYCN | 0.46359 |
| 11134 | I.S. | 0.46358 | 11135 | I.S. | 0.46357 | 11136 | BRIP1 | 0.4635 |
| 11137 | I.S. | 0.46346 | 11138 | I.S. | 0.46344 | 11139 | I.S. | 0.46342 |
| 11140 | I.S. | 0.46341 | 11141 | I.S. | 0.46336 | 11142 | I.S. | 0.46333 |
| 11143 | PREX2 | 0.4633 | 11144 | I.S. | 0.46327 | 11145 | MAGI2 | 0.46323 |
| 11146 | BCORL1 | 0.46322 | 11147 | I.S. | 0.46322 | 11148 | I.S. | 0.46322 |
| 11149 | RAD51 | 0.46319 | 11150 | KDR | 0.46315 | 11151 | NOTCH2 | 0.46312 |
| 11152 | I.S. | 0.46312 | 11153 | I.S. | 0.4631 | 11154 | IDH1 | 0.46307 |
| 11155 | I.S. | 0.46298 | 11156 | I.S. | 0.46293 | 11157 | BCYRN1, | 0.46289 |
| | | | | | | | TAF1 | |
| 11158 | I.S. | 0.46287 | 11159 | I.S. | 0.46283 | 11160 | I.S. | 0.46279 |
| 11161 | I.S. | 0.46274 | 11162 | I.S. | 0.46274 | 11163 | PAX5 | 0.46269 |
| 11164 | I.S. | 0.46262 | 11165 | I.S. | 0.46261 | 11166 | I.S. | 0.46261 |
| 11167 | I.S. | 0.46255 | 11168 | ATR | 0.46253 | 11169 | I.S. | 0.46252 |
| 11170 | I.S. | 0.46252 | 11171 | XPO1 | 0.46249 | 11172 | ATRX | 0.46247 |
| 11173 | I.S. | 0.46246 | 11174 | I.S. | 0.46235 | 11175 | I.S. | 0.46232 |
| 11176 | I.S. | 0.46229 | 11177 | I.S. | 0.46229 | 11178 | SLX4 | 0.46226 |
| 11179 | I.S. | 0.46225 | 11180 | EPHA3 | 0.46222 | 11181 | I.S. | 0.4622 |
| 11182 | I.S. | 0.46219 | 11183 | I.S. | 0.46208 | 11184 | NOTCH2 | 0.46205 |
| 11185 | I.S. | 0.46203 | 11186 | JAK2 | 0.46202 | 11187 | I.S. | 0.46202 |
| 11188 | I.S. | 0.462 | 11189 | I.S. | 0.462 | 11190 | NSD1 | 0.46199 |
| 11191 | PDK1 | 0.46197 | 11192 | I.S. | 0.46195 | 11193 | RB1 | 0.46195 |
| 11194 | PHF6 | 0.46194 | 11195 | I.S. | 0.46193 | 11196 | I.S. | 0.4619 |
| 11197 | ATRX | 0.46187 | 11198 | I.S. | 0.46185 | 11199 | I.S. | 0.46181 |
| 11200 | I.S. | 0.46181 | 11201 | I.S. | 0.46181 | 11202 | I.S. | 0.4618 |
| 11203 | CBLB | 0.46176 | 11204 | CYLD | 0.46173 | 11205 | I.S. | 0.46172 |
| 11206 | I.S. | 0.46171 | 11207 | MSH2 | 0.4617 | 11208 | CHD4 | 0.46167 |
| 11209 | I.S. | 0.46167 | 11210 | CTC1 | 0.46167 | 11211 | FBXW7 | 0.46164 |
| 11212 | NOTCH2 | 0.46161 | 11213 | I.S. | 0.46142 | 11214 | I.S. | 0.46142 |
| 11215 | I.S. | 0.4614 | 11216 | JAK1 | 0.4614 | 11217 | PIK3CB | 0.46137 |
| 11218 | XPO1 | 0.46135 | 11219 | EPHA7 | 0.46134 | 11220 | I.S. | 0.46133 |
| 11221 | ARID2 | 0.46126 | 11222 | I.S. | 0.46125 | 11223 | I.S. | 0.46125 |
| 11224 | ARAF | 0.46116 | 11225 | I.S. | 0.46116 | 11226 | I.S. | 0.46113 |
| 11227 | I.S. | 0.46111 | 11228 | ARID2 | 0.4611 | 11229 | NOTCH2 | 0.46107 |
| 11230 | I.S. | 0.46105 | 11231 | I.S. | 0.46104 | 11232 | IKZF1 | 0.46093 |
| 11233 | PIK3R1 | 0.46092 | 11234 | KMT2B | 0.46092 | 11235 | I.S. | 0.46084 |
| 11236 | I.S. | 0.46077 | 11237 | GFI1 | 0.46072 | 11238 | DICER1 | 0.46071 |
| 11239 | CYLD | 0.46069 | 11240 | I.S. | 0.46068 | 11241 | STAG2 | 0.46066 |
| 11242 | I.S. | 0.46065 | 11243 | I.S. | 0.46063 | 11244 | I.S. | 0.46063 |
| 11245 | CHEK2 | 0.46062 | 11246 | I.S. | 0.4606 | 11247 | KMT2A | 0.46058 |
| 11248 | FANCB | 0.46055 | 11249 | I.S. | 0.46054 | 11250 | I.S. | 0.46045 |
| 11251 | B2M | 0.46044 | 11252 | I.S. | 0.46041 | 11253 | CHD2 | 0.46041 |
| 11254 | I.S. | 0.4604 | 11255 | RANBP2 | 0.46036 | 11256 | I.S. | 0.46032 |
| 11257 | MSH2 | 0.4603 | 11258 | RANBP2 | 0.4603 | 11259 | I.S. | 0.46029 |
| 11260 | RANBP2 | 0.46028 | 11261 | I.S. | 0.46027 | 11262 | I.S. | 0.46027 |
| 11263 | RANBP2 | 0.46021 | 11264 | I.S. | 0.46019 | 11265 | I.S. | 0.46018 |
| 11266 | I.S. | 0.46015 | 11267 | SMC1A | 0.4601 | 11268 | SH2D2A, | 0.46004 |
| | | | | | | | NTRK1 | |
| 11269 | I.S. | 0.46002 | 11270 | PIM1 | 0.46 | 11271 | I.S. | 0.45997 |
| 11272 | BCYRN1, | 0.45997 | 11273 | DKC1 | 0.45995 | 11274 | LYN | 0.45995 |
| | TAF1 | | | | | | | |
| 11275 | CREBBP | 0.45994 | 11276 | SMC1A, | 0.45992 | 11277 | SF3B1 | 0.45991 |
| | | | | MIR6857 | | | | |
| 11278 | I.S. | 0.4599 | 11279 | I.S. | 0.45985 | 11280 | NTHL1, | 0.45984 |
| | | | | | | | TSC2 | |
| 11281 | I.S. | 0.45982 | 11282 | PDK1 | 0.4598 | 11283 | I.S. | 0.45976 |
| 11284 | I.S. | 0.4597 | 11285 | EMSY | 0.45967 | 11286 | I.S. | 0.45961 |
| 11287 | TCIRG1 | 0.45961 | 11288 | I.S. | 0.45953 | 11289 | I.S. | 0.45947 |
| 11290 | I.S. | 0.45937 | 11291 | MAP3K1 | 0.45928 | 11292 | I.S. | 0.45924 |
| 11293 | I.S. | 0.4592 | 11294 | ARAF | 0.45915 | 11295 | KMT2C | 0.45913 |
| 11296 | APC | 0.45911 | 11297 | I.S. | 0.45906 | 11298 | BIRC3 | 0.45903 |
| 11299 | I.S. | 0.45903 | 11300 | I.S. | 0.45899 | 11301 | RPTOR | 0.45899 |
| 11302 | I.S. | 0.45896 | 11303 | GEN1 | 0.45893 | 11304 | I.S. | 0.45893 |
| 11305 | I.S. | 0.45886 | 11306 | RBM10 | 0.45882 | 11307 | I.S. | 0.45881 |
| 11308 | I.S. | 0.45874 | 11309 | FANCD2 | 0.45873 | 11310 | I.S. | 0.45869 |
| 11311 | I.S. | 0.45867 | 11312 | BRIP1 | 0.45863 | 11313 | I.S. | 0.45862 |
| 11314 | I.S. | 0.45858 | 11315 | I.S. | 0.45854 | 11316 | I.S. | 0.45851 |
| 11317 | PMS1 | 0.45845 | 11318 | FANCD2 | 0.45841 | 11319 | I.S. | 0.4584 |
| 11320 | I.S. | 0.45838 | 11321 | TERT | 0.45837 | 11322 | I.S. | 0.45834 |
| 11323 | I.S. | 0.45834 | 11324 | I.S. | 0.45833 | 11325 | I.S. | 0.45822 |
| 11326 | I.S. | 0.4582 | 11327 | GID4 | 0.45819 | 11328 | IL7R | 0.45814 |
| 11329 | I.S. | 0.45814 | 11330 | PIK3R2 | 0.4581 | 11331 | I.S. | 0.4581 |
| 11332 | I.S. | 0.45808 | 11333 | — | 0.45805 | 11334 | MSH6 | 0.45802 |
| 11335 | PREX2 | 0.45798 | 11336 | EP300 | 0.45796 | 11337 | I.S. | 0.45793 |
| 11338 | I.S. | 0.45792 | 11339 | I.S. | 0.45787 | 11340 | I.S. | 0.45786 |
| 11341 | CBLB | 0.45784 | 11342 | I.S. | 0.4578 | 11343 | I.S. | 0.45777 |
| 11344 | I.S. | 0.45772 | 11345 | I.S. | 0.45768 | 11346 | PAK3 | 0.45766 |
| 11347 | I.S. | 0.45765 | 11348 | I.S. | 0.45765 | 11349 | I.S. | 0.45764 |
| 11350 | SDHC | 0.45764 | 11351 | I.S. | 0.45763 | 11352 | I.S. | 0.45763 |
| 11353 | CUX1 | 0.45762 | 11354 | CBLB | 0.4576 | 11355 | FANCA | 0.4576 |
| 11356 | I.S. | 0.45757 | 11357 | I.S. | 0.45757 | 11358 | MAP3K1 | 0.45756 |
| 11359 | RBM10 | 0.45754 | 11360 | KDM5C | 0.45754 | 11361 | EMSY | 0.45751 |
| 11362 | I.S. | 0.45749 | 11363 | FANCD2 | 0.45743 | 11364 | I.S. | 0.45739 |
| 11365 | I.S. | 0.45727 | 11366 | MAP3K1 | 0.45726 | 11367 | ATG2B | 0.45724 |
| 11368 | I.S. | 0.45723 | 11369 | I.S. | 0.45721 | 11370 | I.S. | 0.45718 |
| 11371 | PRKDC | 0.45709 | 11372 | I.S. | 0.45708 | 11373 | I.S. | 0.45707 |
| 11374 | I.S. | 0.45698 | 11375 | I.S. | 0.45697 | 11376 | I.S. | 0.45691 |
| 11377 | I.S. | 0.45689 | 11378 | SMO | 0.45687 | 11379 | FANCD2 | 0.45686 |
| 11380 | I.S. | 0.4568 | 11381 | I.S. | 0.45679 | 11382 | I.S. | 0.45677 |
| 11383 | I.S. | 0.45674 | 11384 | EP300 | 0.45668 | 11385 | I.S. | 0.45667 |
| 11386 | I.S. | 0.45661 | 11387 | I.S. | 0.45659 | 11388 | SETD2 | 0.45653 |
| 11389 | FLT4 | 0.45649 | 11390 | I.S. | 0.45644 | 11391 | I.S. | 0.45644 |
| 11392 | I.S. | 0.45644 | 11393 | I.S. | 0.45641 | 11394 | I.S. | 0.45638 |
| 11395 | I.S. | 0.45638 | 11396 | PRKAR1A, | 0.45629 | 11397 | SAMD9L | 0.45627 |
| | | | | FAM20A | | | | |
| 11398 | I.S. | 0.45619 | 11399 | I.S. | 0.45617 | 11400 | XRCC2 | 0.45617 |
| 11401 | SUZ12 | 0.45615 | 11402 | I.S. | 0.45614 | 11403 | KMT2D | 0.45612 |
| 11404 | I.S. | 0.45611 | 11405 | STAT3 | 0.45611 | 11406 | I.S. | 0.45611 |
| 11407 | NOTCH3 | 0.45606 | 11408 | ABL2 | 0.45599 | 11409 | MAP2K4 | 0.45597 |
| 11410 | CDK12 | 0.45596 | 11411 | I.S. | 0.45593 | 11412 | PIK3R1 | 0.4559 |
| 11413 | I.S. | 0.4559 | 11414 | SMAD3 | 0.45587 | 11415 | I.S. | 0.45587 |
| 11416 | I.S. | 0.45587 | 11417 | I.S. | 0.45583 | 11418 | I.S. | 0.45582 |
| 11419 | I.S. | 0.45579 | 11420 | I.S. | 0.45577 | 11421 | AMER1 | 0.45576 |
| 11422 | I.S. | 0.45573 | 11423 | I.S. | 0.45572 | 11424 | I.S. | 0.4557 |
| 11425 | CHD2 | 0.45569 | 11426 | I.S. | 0.45569 | 11427 | I.S. | 0.45567 |
| 11428 | I.S. | 0.45558 | 11429 | I.S. | 0.45558 | 11430 | I.S. | 0.45557 |
| 11431 | I.S. | 0.45555 | 11432 | I.S. | 0.45555 | 11433 | CDK6 | 0.45555 |
| 11434 | NFE2L2 | 0.4555 | 11435 | I.S. | 0.45549 | 11436 | I.S. | 0.45549 |
| 11437 | PRKAR1A | 0.45548 | 11438 | CUL3 | 0.45546 | 11439 | I.S. | 0.45546 |
| 11440 | I.S. | 0.45543 | 11441 | DOT1L | 0.45543 | 11442 | I.S. | 0.45541 |
| 11443 | KDM6A | 0.4554 | 11444 | I.S. | 0.45537 | 11445 | TCIRG1 | 0.45537 |
| 11446 | I.S. | 0.45537 | 11447 | I.S. | 0.45536 | 11448 | I.S. | 0.45532 |
| 11449 | I.S. | 0.45532 | 11450 | I.S. | 0.4553 | 11451 | I.S. | 0.45528 |
| 11452 | I.S. | 0.45528 | 11453 | I.S. | 0.45528 | 11454 | I.S. | 0.45527 |
| 11455 | I.S. | 0.45524 | 11456 | GSK3B | 0.45522 | 11457 | NFKBIA | 0.45521 |
| 11458 | NUP93 | 0.45515 | 11459 | I.S. | 0.45514 | 11460 | I.S. | 0.45513 |
| 11461 | I.S. | 0.45511 | 11462 | I.S. | 0.45495 | 11463 | MAGI2 | 0.4549 |
| 11464 | I.S. | 0.45489 | 11465 | I.S. | 0.45489 | 11466 | I.S. | 0.45487 |
| 11467 | I.S. | 0.45483 | 11468 | POT1 | 0.45481 | 11469 | I.S. | 0.45481 |
| 11470 | I.S. | 0.4548 | 11471 | I.S. | 0.45478 | 11472 | INPP4B | 0.45469 |
| 11473 | GATA1 | 0.45466 | 11474 | I.S. | 0.4546 | 11475 | I.S. | 0.45459 |
| 11476 | BCORL1 | 0.45457 | 11477 | NFE2L2 | 0.45451 | 11478 | I.S. | 0.45451 |
| 11479 | I.S. | 0.45443 | 11480 | I.S. | 0.45442 | 11481 | NOTCH2 | 0.4544 |
| 11482 | I.S. | 0.45439 | 11483 | I.S. | 0.45438 | 11484 | I.S. | 0.45438 |
| 11485 | I.S. | 0.45433 | 11486 | I.S. | 0.4543 | 11487 | I.S. | 0.4543 |
| 11488 | I.S. | 0.4543 | 11489 | I.S. | 0.45427 | 11490 | SETD2 | 0.45424 |
| 11491 | I.S. | 0.45422 | 11492 | I.S. | 0.45418 | 11493 | I.S. | 0.4541 |
| 11494 | ATR | 0.45406 | 11495 | IRS2 | 0.45405 | 11496 | I.S. | 0.45404 |
| 11497 | I.S. | 0.45401 | 11498 | I.S. | 0.45401 | 11499 | EPAS1 | 0.45396 |
| 11500 | I.S. | 0.45391 | 11501 | I.S. | 0.45388 | 11502 | I.S. | 0.45387 |
| 11503 | NLRP3 | 0.45386 | 11504 | I.S. | 0.45385 | 11505 | GNAS | 0.45382 |
| 11506 | I.S. | 0.45382 | 11507 | AR | 0.4538 | 11508 | I.S. | 0.4538 |
| 11509 | I.S. | 0.45376 | 11510 | I.S. | 0.45374 | 11511 | I.S. | 0.45371 |
| 11512 | AKT1 | 0.4537 | 11513 | MSH6 | 0.45368 | 11514 | I.S. | 0.45368 |
| 11515 | TERF1 | 0.45367 | 11516 | I.S. | 0.45367 | 11517 | RBM10 | 0.45362 |
| 11518 | I.S. | 0.45361 | 11519 | I.S. | 0.4536 | 11520 | I.S. | 0.45356 |
| 11521 | RANBP2 | 0.45355 | 11522 | I.S. | 0.45353 | 11523 | I.S. | 0.45346 |
| 11524 | I.S. | 0.45344 | 11525 | I.S. | 0.45343 | 11526 | KIT | 0.45336 |
| 11527 | I.S. | 0.45334 | 11528 | I.S. | 0.45332 | 11529 | I.S. | 0.45332 |
| 11530 | I.S. | 0.45332 | 11531 | AXIN1 | 0.45331 | 11532 | I.S. | 0.45326 |
| 11533 | I.S. | 0.45323 | 11534 | I.S. | 0.45323 | 11535 | I.S. | 0.45322 |
| 11536 | MEN1 | 0.45318 | 11537 | I.S. | 0.45315 | 11538 | I.S. | 0.45315 |
| 11539 | I.S. | 0.45314 | 11540 | I.S. | 0.45311 | 11541 | I.S. | 0.45309 |
| 11542 | FANCA | 0.45308 | 11543 | I.S. | 0.45305 | 11544 | I.S. | 0.45305 |
| 11545 | I.S. | 0.45305 | 11546 | I.S. | 0.45304 | 11547 | I.S. | 0.45302 |
| 11548 | I.S. | 0.45296 | 11549 | TERT | 0.45287 | 11550 | I.S. | 0.45287 |
| 11551 | I.S. | 0.45285 | 11552 | I.S. | 0.4528 | 11553 | I.S. | 0.45275 |
| 11554 | I.S. | 0.45271 | 11555 | PRKDC | 0.45268 | 11556 | DKC1 | 0.45264 |
| 11557 | I.S. | 0.45257 | 11558 | I.S. | 0.45257 | 11559 | I.S. | 0.45256 |
| 11560 | I.S. | 0.45254 | 11561 | I.S. | 0.45253 | 11562 | I.S. | 0.45252 |
| 11563 | I.S. | 0.45249 | 11564 | I.S. | 0.45242 | 11565 | I.S. | 0.45242 |
| 11566 | GRIN2A | 0.4524 | 11567 | NRAS | 0.45234 | 11568 | SEC23B | 0.45231 |
| 11569 | ABCB7 | 0.45228 | 11570 | PALB2 | 0.45228 | 11571 | BAP1 | 0.45221 |
| 11572 | I.S. | 0.4522 | 11573 | I.S. | 0.45219 | 11574 | I.S. | 0.45219 |
| 11575 | I.S. | 0.45216 | 11576 | I.S. | 0.45216 | 11577 | I.S. | 0.45214 |
| 11578 | ABL2 | 0.45208 | 11579 | I.S. | 0.45207 | 11580 | I.S. | 0.45205 |
| 11581 | RBM10 | 0.45198 | 11582 | CUX1 | 0.45195 | 11583 | I.S. | 0.45195 |
| 11584 | I.S. | 0.45195 | 11585 | I.S. | 0.45194 | 11586 | PMS2 | 0.45193 |
| 11587 | SPEN | 0.4519 | 11588 | I.S. | 0.4519 | 11589 | I.S. | 0.4519 |
| 11590 | DDX11 | 0.45187 | 11591 | FANCC | 0.45187 | 11592 | I.S. | 0.45184 |
| 11593 | I.S. | 0.4518 | 11594 | ZNF703 | 0.45178 | 11595 | RAD51 | 0.45175 |
| 11596 | PDGFRB | 0.45169 | 11597 | I.S. | 0.45168 | 11598 | I.S. | 0.45166 |
| 11599 | I.S. | 0.45164 | 11600 | ANKRD26 | 0.45163 | 11601 | I.S. | 0.45163 |
| 11602 | I.S. | 0.4516 | 11603 | I.S. | 0.45157 | 11604 | I.S. | 0.45156 |
| 11605 | RAF1 | 0.45153 | 11606 | I.S. | 0.4515 | 11607 | I.S. | 0.45149 |
| 11608 | ERCC4 | 0.45148 | 11609 | I.S. | 0.45141 | 11610 | I.S. | 0.4514 |
| 11611 | I.S. | 0.45135 | 11612 | I.S. | 0.45133 | 11613 | STAT3 | 0.4513 |
| 11614 | — | 0.4513 | 11615 | I.S. | 0.45129 | 11616 | I.S. | 0.45127 |
| 11617 | I.S. | 0.45124 | 11618 | MED12 | 0.45124 | 11619 | PIK3CG | 0.45121 |
| 11620 | I.S. | 0.45121 | 11621 | JAK2 | 0.45116 | 11622 | I.S. | 0.45115 |
| 11623 | I.S. | 0.45112 | 11624 | RAF1 | 0.4511 | 11625 | I.S. | 0.4511 |
| 11626 | I.S. | 0.45107 | 11627 | I.S. | 0.45107 | 11628 | I.S. | 0.45098 |
| 11629 | NF1 | 0.4509 | 11630 | I.S. | 0.4509 | 11631 | I.S. | 0.45082 |
| 11632 | DDX11 | 0.45082 | 11633 | I.S. | 0.45078 | 11634 | I.S. | 0.45077 |
| 11635 | MEN1 | 0.45077 | 11636 | I.S. | 0.45076 | 11637 | I.S. | 0.45066 |
| 11638 | PALB2 | 0.45065 | 11639 | KIF23 | 0.45056 | 11640 | I.S. | 0.45053 |
| 11641 | I.S. | 0.4505 | 11642 | I.S. | 0.45044 | 11643 | TERT | 0.45044 |
| 11644 | CYLD | 0.45035 | 11645 | I.S. | 0.45031 | 11646 | I.S. | 0.45029 |
| 11647 | I.S. | 0.45029 | 11648 | I.S. | 0.45026 | 11649 | PIK3CB | 0.45025 |
| 11650 | I.S. | 0.4502 | 11651 | I.S. | 0.45017 | 11652 | I.S. | 0.45017 |
| 11653 | I.S. | 0.45015 | 11654 | ARID1A | 0.45015 | 11655 | FGFR2 | 0.45013 |
| 11656 | I.S. | 0.45011 | 11657 | I.S. | 0.45009 | 11658 | I.S. | 0.44997 |
| 11659 | I.S. | 0.44996 | 11660 | FANCD2 | 0.44993 | 11661 | I.S. | 0.44993 |
| 11662 | — | 0.44988 | 11663 | KEL | 0.44985 | 11664 | I.S. | 0.44985 |
| 11665 | ANKRD26 | 0.44982 | 11666 | NOTCH3 | 0.44976 | 11667 | PRKDC | 0.44975 |
| 11668 | I.S. | 0.44972 | 11669 | FANCI | 0.44967 | 11670 | SETD2 | 0.44964 |
| 11671 | I.S. | 0.44962 | 11672 | PTCH1 | 0.44961 | 11673 | KDM5C | 0.44961 |
| 11674 | CBL | 0.44961 | 11675 | I.S. | 0.44956 | 11676 | I.S. | 0.44956 |
| 11677 | I.S. | 0.44955 | 11678 | BCYRN1, | 0.44955 | 11679 | I.S. | 0.44952 |
| | | | | TAF1 | | | | |
| 11680 | I.S. | 0.44951 | 11681 | RBBP6 | 0.44948 | 11682 | I.S. | 0.44948 |
| 11683 | I.S. | 0.44947 | 11684 | I.S. | 0.44946 | 11685 | I.S. | 0.44946 |
| 11686 | ATRX | 0.44943 | 11687 | I.S. | 0.44941 | 11688 | I.S. | 0.44936 |
| 11689 | I.S. | 0.44933 | 11690 | EPHA7 | 0.44932 | 11691 | EP300 | 0.44922 |
| 11692 | BCR | 0.44921 | 11693 | KEAP1 | 0.44919 | 11694 | I.S. | 0.44916 |
| 11695 | NOTCH3 | 0.44916 | 11696 | FANCG | 0.44915 | 11697 | BLM | 0.44915 |
| 11698 | PLCG2 | 0.44914 | 11699 | RANBP2 | 0.44913 | 11700 | I.S. | 0.44913 |
| 11701 | PTEN | 0.44911 | 11702 | MED12 | 0.44906 | 11703 | I.S. | 0.44904 |
| 11704 | I.S. | 0.44902 | 11705 | RET | 0.44901 | 11706 | RB1 | 0.44898 |
| 11707 | I.S. | 0.44896 | 11708 | I.S. | 0.44895 | 11709 | PBRM1 | 0.44891 |
| 11710 | I.S. | 0.44883 | 11711 | I.S. | 0.44881 | 11712 | I.S. | 0.44878 |
| 11713 | I.S. | 0.44871 | 11714 | PMS1 | 0.44867 | 11715 | I.S. | 0.44866 |
| 11716 | I.S. | 0.44863 | 11717 | STAG2 | 0.4486 | 11718 | I.S. | 0.44858 |
| 11719 | I.S. | 0.44853 | 11720 | I.S. | 0.44851 | 11721 | I.S. | 0.44849 |
| 11722 | I.S. | 0.44846 | 11723 | SPTA1 | 0.44845 | 11724 | MAPK1 | 0.44845 |
| 11725 | I.S. | 0.44844 | 11726 | I.S. | 0.44842 | 11727 | I.S. | 0.44842 |
| 11728 | I.S. | 0.44839 | 11729 | I.S. | 0.44836 | 11730 | I.S. | 0.44835 |
| 11731 | NTRK2 | 0.4483 | 11732 | I.S. | 0.4483 | 11733 | I.S. | 0.4483 |
| 11734 | I.S. | 0.44827 | 11735 | RUNX1 | 0.44826 | 11736 | I.S. | 0.44824 |
| 11737 | I.S. | 0.44822 | 11738 | PIK3R1 | 0.44821 | 11739 | I.S. | 0.44821 |
| 11740 | I.S. | 0.4482 | 11741 | I.S. | 0.44817 | 11742 | CTC1 | 0.44815 |
| 11743 | I.S. | 0.44815 | 11744 | I.S. | 0.44813 | 11745 | I.S. | 0.44812 |
| 11746 | TERT | 0.44806 | 11747 | I.S. | 0.44801 | 11748 | I.S. | 0.44797 |
| 11749 | CBLB | 0.44797 | 11750 | I.S. | 0.44795 | 11751 | I.S. | 0.44795 |
| 11752 | I.S. | 0.44791 | 11753 | ARID1A | 0.4479 | 11754 | I.S. | 0.44786 |
| 11755 | ARAF | 0.44783 | 11756 | CUL3 | 0.44781 | 11757 | I.S. | 0.4478 |
| 11758 | PRKDC | 0.4478 | 11759 | I.S. | 0.44776 | 11760 | I.S. | 0.44776 |
| 11761 | ARAF | 0.44771 | 11762 | I.S. | 0.44768 | 11763 | PTPN11 | 0.44768 |
| 11764 | I.S. | 0.44763 | 11765 | I.S. | 0.4476 | 11766 | I.S. | 0.4476 |
| 11767 | I.S. | 0.44759 | 11768 | I.S. | 0.44755 | 11769 | KMT2A | 0.44754 |
| 11770 | I.S. | 0.44751 | 11771 | I.S. | 0.4475 | 11772 | SAMD9L | 0.44746 |
| 11773 | I.S. | 0.44744 | 11774 | I.S. | 0.44744 | 11775 | BTK | 0.44741 |
| 11776 | RAD50 | 0.44735 | 11777 | NRAS | 0.44735 | 11778 | I.S. | 0.44734 |
| 11779 | I.S. | 0.44733 | 11780 | I.S. | 0.44732 | 11781 | I.S. | 0.44731 |
| 11782 | I.S. | 0.44729 | 11783 | I.S. | 0.44728 | 11784 | I.S. | 0.44727 |
| 11785 | I.S. | 0.44726 | 11786 | I.S. | 0.44712 | 11787 | PMS1 | 0.44711 |
| 11788 | I.S. | 0.4471 | 11789 | I.S. | 0.44708 | 11790 | RICTOR | 0.44703 |
| 11791 | I.S. | 0.4469 | 11792 | I.S. | 0.44688 | 11793 | LRP1B | 0.44687 |
| 11794 | MITF | 0.44682 | 11795 | I.S. | 0.44682 | 11796 | I.S. | 0.44681 |
| 11797 | I.S. | 0.44676 | 11798 | LPIN2 | 0.4467 | 11799 | BCYRN1, | 0.44664 |
| | | | | | | | TAF1 | |
| 11800 | I.S. | 0.44664 | 11801 | FGF19 | 0.4466 | 11802 | I.S. | 0.44658 |
| 11803 | AXIN2 | 0.44658 | 11804 | SYK | 0.44658 | 11805 | I.S. | 0.44653 |
| 11806 | I.S. | 0.44648 | 11807 | LRP1B | 0.44643 | 11808 | I.S. | 0.4464 |
| 11809 | I.S. | 0.44639 | 11810 | LRP1B | 0.44639 | 11811 | I.S. | 0.44637 |
| 11812 | ATR | 0.44637 | 11813 | I.S. | 0.44635 | 11814 | ATRX | 0.44634 |
| 11815 | I.S. | 0.44634 | 11816 | I.S. | 0.44634 | 11817 | I.S. | 0.44628 |
| 11818 | PRKDC | 0.44626 | 11819 | I.S. | 0.44624 | 11820 | I.S. | 0.44623 |
| 11821 | I.S. | 0.44616 | 11822 | ARID1A | 0.44616 | 11823 | NROB1 | 0.44613 |
| 11824 | I.S. | 0.44613 | 11825 | I.S. | 0.44611 | 11826 | RET | 0.4461 |
| 11827 | BAP1 | 0.44609 | 11828 | I.S. | 0.44609 | 11829 | I.S. | 0.44607 |
| 11830 | I.S. | 0.44606 | 11831 | I.S. | 0.44598 | 11832 | I.S. | 0.44595 |
| 11833 | I.S. | 0.44594 | 11834 | ERBB2 | 0.44592 | 11835 | I.S. | 0.4459 |
| 11836 | I.S. | 0.44588 | 11837 | EPAS1 | 0.44584 | 11838 | I.S. | 0.44584 |
| 11839 | I.S. | 0.44583 | 11840 | I.S. | 0.44578 | 11841 | I.S. | 0.44577 |
| 11842 | I.S. | 0.44574 | 11843 | I.S. | 0.44574 | 11844 | I.S. | 0.44572 |
| 11845 | I.S. | 0.44572 | 11846 | I.S. | 0.44571 | 11847 | TOP2A | 0.44568 |
| 11848 | I.S. | 0.44566 | 11849 | I.S. | 0.44565 | 11850 | I.S. | 0.44563 |
| 11851 | I.S. | 0.44563 | 11852 | MED12 | 0.44563 | 11853 | I.S. | 0.44559 |
| 11854 | I.S. | 0.44557 | 11855 | I.S. | 0.44554 | 11856 | I.S. | 0.44551 |
| 11857 | I.S. | 0.44548 | 11858 | I.S. | 0.44542 | 11859 | I.S. | 0.44542 |
| 11860 | I.S. | 0.44541 | 11861 | MDM4 | 0.44539 | 11862 | APC | 0.44538 |
| 11863 | ATRX | 0.44536 | 11864 | I.S. | 0.44536 | 11865 | I.S. | 0.44536 |
| 11866 | CUL3 | 0.44536 | 11867 | SAMD9L | 0.44533 | 11868 | I.S. | 0.44533 |
| 11869 | I.S. | 0.44533 | 11870 | I.S. | 0.4453 | 11871 | I.S. | 0.4453 |
| 11872 | I.S. | 0.44525 | 11873 | I.S. | 0.44522 | 11874 | RANBP2 | 0.44521 |
| 11875 | FUBP1 | 0.44515 | 11876 | ASXL1 | 0.44509 | 11877 | XPO1 | 0.44509 |
| 11878 | I.S. | 0.44507 | 11879 | SMAD3 | 0.44503 | 11880 | I.S. | 0.44501 |
| 11881 | FGF19 | 0.44501 | 11882 | I.S. | 0.445 | 11883 | U2AF1, | 0.44499 |
| | | | | | | | U2AF1L5 | |
| 11884 | I.S. | 0.44497 | 11885 | GEN1 | 0.44497 | 11886 | FANCG | 0.44497 |
| 11887 | I.S. | 0.44497 | 11888 | I.S. | 0.44497 | 11889 | I.S. | 0.44496 |
| 11890 | I.S. | 0.44494 | 11891 | KDR | 0.44492 | 11892 | BIRC3 | 0.44492 |
| 11893 | JAK2 | 0.44491 | 11894 | I.S. | 0.44486 | 11895 | I.S. | 0.44485 |
| 11896 | I.S. | 0.44484 | 11897 | KDM5C | 0.44483 | 11898 | LRP1B | 0.44481 |
| 11899 | I.S. | 0.44477 | 11900 | I.S. | 0.44474 | 11901 | I.S. | 0.44473 |
| 11902 | I.S. | 0.44471 | 11903 | EPHA3 | 0.44471 | 11904 | CARD11 | 0.44468 |
| 11905 | RHEB | 0.44464 | 11906 | ATRX | 0.44462 | 11907 | I.S. | 0.44459 |
| 11908 | I.S. | 0.44458 | 11909 | GRM3 | 0.44456 | 11910 | I.S. | 0.44456 |
| 11911 | PRKDC | 0.44451 | 11912 | AXIN2 | 0.44446 | 11913 | I.S. | 0.44445 |
| 11914 | RBBP6 | 0.44444 | 11915 | I.S. | 0.44443 | 11916 | I.S. | 0.44443 |
| 11917 | I.S. | 0.4444 | 11918 | I.S. | 0.44439 | 11919 | EZH2 | 0.44439 |
| 11920 | I.S. | 0.44438 | 11921 | I.S. | 0.44438 | 11922 | I.S. | 0.44437 |
| 11923 | RAD51D | 0.44429 | 11924 | I.S. | 0.44423 | 11925 | I.S. | 0.44423 |
| 11926 | BCYRN1, | 0.44421 | 11927 | BCORL1 | 0.4442 | 11928 | I.S. | 0.4442 |
| | TAF1 | | | | | | | |
| 11929 | PRKCI | 0.44414 | 11930 | I.S. | 0.44409 | 11931 | I.S. | 0.44407 |
| 11932 | I.S. | 0.44404 | 11933 | I.S. | 0.44402 | 11934 | JAK3 | 0.44397 |
| 11935 | I.S. | 0.44396 | 11936 | I.S. | 0.44396 | 11937 | I.S. | 0.44394 |
| 11938 | I.S. | 0.44389 | 11939 | I.S. | 0.44388 | 11940 | STAT4 | 0.44384 |
| 11941 | I.S. | 0.44375 | 11942 | I.S. | 0.44373 | 11943 | I.S. | 0.44372 |
| 11944 | SLX4 | 0.44366 | 11945 | I.S. | 0.4436 | 11946 | ROS1 | 0.44358 |
| 11947 | EP300 | 0.44358 | 11948 | I.S. | 0.44357 | 11949 | I.S. | 0.44357 |
| 11950 | I.S. | 0.44354 | 11951 | I.S. | 0.44352 | 11952 | I.S. | 0.44348 |
| 11953 | I.S. | 0.44342 | 11954 | BCOR | 0.44342 | 11955 | I.S. | 0.44342 |
| 11956 | I.S. | 0.44341 | 11957 | I.S. | 0.44331 | 11958 | I.S. | 0.44331 |
| 11959 | I.S. | 0.44325 | 11960 | MSH6 | 0.44322 | 11961 | I.S. | 0.4432 |
| 11962 | I.S. | 0.44319 | 11963 | BCOR | 0.44316 | 11964 | I.S. | 0.44315 |
| 11965 | I.S. | 0.44307 | 11966 | I.S. | 0.44304 | 11967 | I.S. | 0.44298 |
| 11968 | ERBB4 | 0.44295 | 11969 | I.S. | 0.44292 | 11970 | I.S. | 0.44289 |
| 11971 | BCORL1 | 0.44286 | 11972 | I.S. | 0.44286 | 11973 | I.S. | 0.44285 |
| 11974 | I.S. | 0.44284 | 11975 | I.S. | 0.44278 | 11976 | TCIRG1 | 0.44278 |
| 11977 | I.S. | 0.44275 | 11978 | TOP1 | 0.44275 | 11979 | LRP1B | 0.4427 |
| 11980 | I.S. | 0.44265 | 11981 | I.S. | 0.44265 | 11982 | SBF2 | 0.44256 |
| 11983 | I.S. | 0.44256 | 11984 | I.S. | 0.44254 | 11985 | I.S. | 0.44254 |
| 11986 | I.S. | 0.4425 | 11987 | I.S. | 0.4425 | 11988 | I.S. | 0.44246 |
| 11989 | PREX2 | 0.44245 | 11990 | I.S. | 0.44241 | 11991 | FANCG | 0.44238 |
| 11992 | RAF1 | 0.44236 | 11993 | PRKDC | 0.44233 | 11994 | I.S. | 0.44233 |
| 11995 | — | 0.44233 | 11996 | I.S. | 0.44232 | 11997 | I.S. | 0.44231 |
| 11998 | I.S. | 0.44229 | 11999 | TSC1 | 0.44224 | 12000 | I.S. | 0.44224 |
| 12001 | I.S. | 0.44221 | 12002 | I.S. | 0.44218 | 12003 | I.S. | 0.44216 |
| 12004 | MAP3K1 | 0.44215 | 12005 | I.S. | 0.44214 | 12006 | I.S. | 0.44209 |
| 12007 | MED12 | 0.44206 | 12008 | I.S. | 0.44203 | 12009 | MEFV | 0.44203 |
| 12010 | I.S. | 0.44197 | 12011 | I.S. | 0.44195 | 12012 | I.S. | 0.44193 |
| 12013 | I.S. | 0.44192 | 12014 | I.S. | 0.4419 | 12015 | BMPR1A | 0.44188 |
| 12016 | I.S. | 0.44187 | 12017 | PALB2 | 0.44185 | 12018 | I.S. | 0.44185 |
| 12019 | I.S. | 0.44182 | 12020 | I.S. | 0.44182 | 12021 | I.S. | 0.4418 |
| 12022 | BCL2L2 | 0.4418 | 12023 | I.S. | 0.44177 | 12024 | I.S. | 0.4417 |
| 12025 | SETD2 | 0.44168 | 12026 | I.S. | 0.44168 | 12027 | I.S. | 0.44165 |
| 12028 | I.S. | 0.44164 | 12029 | I.S. | 0.4416 | 12030 | I.S. | 0.44159 |
| 12031 | I.S. | 0.4415 | 12032 | I.S. | 0.4415 | 12033 | I.S. | 0.4415 |
| 12034 | POLE | 0.44147 | 12035 | I.S. | 0.44147 | 12036 | TOP1 | 0.44143 |
| 12037 | I.S. | 0.44139 | 12038 | I.S. | 0.44127 | 12039 | I.S. | 0.44112 |
| 12040 | I.S. | 0.44108 | 12041 | HGF | 0.44108 | 12042 | I.S. | 0.44105 |
| 12043 | I.S. | 0.44105 | 12044 | I.S. | 0.44103 | 12045 | I.S. | 0.441 |
| 12046 | I.S. | 0.44099 | 12047 | I.S. | 0.44097 | 12048 | I.S. | 0.4409 |
| 12049 | IL7R | 0.44088 | 12050 | I.S. | 0.44087 | 12051 | I.S. | 0.44082 |
| 12052 | I.S. | 0.44082 | 12053 | I.S. | 0.44081 | 12054 | BARD1 | 0.44078 |
| 12055 | I.S. | 0.44078 | 12056 | MUTYH | 0.44076 | 12057 | I.S. | 0.44076 |
| 12058 | FANCD2 | 0.44076 | 12059 | I.S. | 0.44073 | 12060 | I.S. | 0.44073 |
| 12061 | MED12 | 0.44067 | 12062 | I.S. | 0.44061 | 12063 | ZRSR2 | 0.44061 |
| 12064 | I.S. | 0.4406 | 12065 | I.S. | 0.44059 | 12066 | I.S. | 0.44052 |
| 12067 | I.S. | 0.44052 | 12068 | I.S. | 0.44052 | 12069 | I.S. | 0.44052 |
| 12070 | NOTCH1 | 0.44051 | 12071 | GSK3B | 0.44047 | 12072 | FANCB | 0.44046 |
| 12073 | I.S. | 0.44045 | 12074 | I.S. | 0.44045 | 12075 | I.S. | 0.44043 |
| 12076 | I.S. | 0.44043 | 12077 | I.S. | 0.4404 | 12078 | MED12 | 0.4404 |
| 12079 | I.S. | 0.44038 | 12080 | I.S. | 0.44036 | 12081 | I.S. | 0.44036 |
| 12082 | I.S. | 0.44035 | 12083 | I.S. | 0.44034 | 12084 | ANKRD26 | 0.4403 |
| 12085 | BRAF | 0.44027 | 12086 | PMS2 | 0.44027 | 12087 | I.S. | 0.44025 |
| 12088 | FBXW7 | 0.44023 | 12089 | TAL1 | 0.44019 | 12090 | ATRX | 0.44018 |
| 12091 | I.S. | 0.44015 | 12092 | ARAF | 0.44013 | 12093 | I.S. | 0.44013 |
| 12094 | I.S. | 0.44012 | 12095 | I.S. | 0.44011 | 12096 | S. | 0.4401 |
| 12097 | I.S. | 0.44006 | 12098 | I.S. | 0.44004 | 12099 | I.S. | 0.43999 |
| 12100 | BTK | 0.43998 | 12101 | I.S. | 0.43993 | 12102 | NOTCH3 | 0.43991 |
| 12103 | I.S. | 0.4399 | 12104 | I.S. | 0.4399 | 12105 | I.S. | 0.43989 |
| 12106 | I.S. | 0.43989 | 12107 | U2AF1, | 0.43987 | 12108 | STAT6 | 0.43987 |
| | | | | U2AF1L5 | | | | |
| 12109 | I.S. | 0.43984 | 12110 | KMT2A | 0.43983 | 12111 | I.S. | 0.43981 |
| 12112 | I.S. | 0.4398 | 12113 | I.S. | 0.4398 | 12114 | I.S. | 0.43974 |
| 12115 | I.S. | 0.4397 | 12116 | I.S. | 0.43969 | 12117 | I.S. | 0.43966 |
| 12118 | RAD54L | 0.43965 | 12119 | I.S. | 0.43965 | 12120 | I.S. | 0.43958 |
| 12121 | I.S. | 0.43957 | 12122 | I.S. | 0.43956 | 12123 | EZH2 | 0.43953 |
| 12124 | NFE2L2 | 0.43951 | 12125 | I.S. | 0.43944 | 12126 | ABL1 | 0.43941 |
| 12127 | GLI2 | 0.43941 | 12128 | I.S. | 0.43939 | 12129 | ATG2B | 0.43939 |
| 12130 | KDR | 0.43933 | 12131 | BRD4 | 0.43932 | 12132 | I.S. | 0.4393 |
| 12133 | ATR | 0.4393 | 12134 | I.S. | 0.43927 | 12135 | I.S. | 0.43925 |
| 12136 | I.S. | 0.43925 | 12137 | KMT2D | 0.43924 | 12138 | I.S. | 0.43918 |
| 12139 | U2AF1, | 0.43918 | 12140 | NUP93 | 0.43917 | 12141 | I.S. | 0.43917 |
| | U2AF1L5 | | | | | | | |
| 12142 | TOP2A | 0.43916 | 12143 | GNAQ | 0.43915 | 12144 | FANCL | 0.43912 |
| 12145 | BCORL1 | 0.43912 | 12146 | I.S. | 0.43906 | 12147 | I.S. | 0.43903 |
| 12148 | I.S. | 0.439 | 12149 | I.S. | 0.43899 | 12150 | RBM10 | 0.43898 |
| 12151 | CHD2 | 0.43897 | 12152 | CALR | 0.43891 | 12153 | CBL | 0.43887 |
| 12154 | I.S. | 0.43887 | 12155 | I.S. | 0.43885 | 12156 | GRIN2A | 0.4388 |
| 12157 | I.S. | 0.43879 | 12158 | I.S. | 0.43879 | 12159 | I.S. | 0.43879 |
| 12160 | I.S. | 0.4387 | 12161 | MED12 | 0.43868 | 12162 | I.S. | 0.43868 |
| 12163 | I.S. | 0.43867 | 12164 | I.S. | 0.43866 | 12165 | KDM6A | 0.43865 |
| 12166 | SMC1A | 0.43865 | 12167 | I.S. | 0.43862 | 12168 | I.S. | 0.43861 |
| 12169 | I.S. | 0.43859 | 12170 | I.S. | 0.43854 | 12171 | PRKDC | 0.43846 |
| 12172 | I.S. | 0.43841 | 12173 | EPHA7 | 0.43841 | 12174 | MTOR | 0.43839 |
| 12175 | I.S. | 0.43836 | 12176 | I.S. | 0.43835 | 12177 | I.S. | 0.43834 |
| 12178 | I.S. | 0.4383 | 12179 | I.S. | 0.43829 | 12180 | I.S. | 0.43829 |
| 12181 | CYLD | 0.43827 | 12182 | I.S. | 0.43826 | 12183 | I.S. | 0.43824 |
| 12184 | I.S. | 0.43822 | 12185 | I.S. | 0.43821 | 12186 | I.S. | 0.4382 |
| 12187 | I.S. | 0.43814 | 12188 | I.S. | 0.43812 | 12189 | I.S. | 0.43807 |
| 12190 | I.S. | 0.43805 | 12191 | I.S. | 0.43805 | 12192 | I.S. | 0.43804 |
| 12193 | FGFR2 | 0.43803 | 12194 | I.S. | 0.43802 | 12195 | I.S. | 0.43802 |
| 12196 | I.S. | 0.438 | 12197 | I.S. | 0.43797 | 12198 | I.S. | 0.43796 |
| 12199 | I.S. | 0.43794 | 12200 | RNF168 | 0.43787 | 12201 | I.S. | 0.43784 |
| 12202 | I.S. | 0.43782 | 12203 | I.S. | 0.43781 | 12204 | CREBBP | 0.4378 |
| 12205 | I.S. | 0.43779 | 12206 | I.S. | 0.43776 | 12207 | I.S. | 0.43772 |
| 12208 | I.S. | 0.4377 | 12209 | AXIN2 | 0.4377 | 12210 | CHD4 | 0.43769 |
| 12211 | CREBBP | 0.43769 | 12212 | VRK2, | 0.43767 | 12213 | KMT2D | 0.43767 |
| | | | | FANCL | | | | |
| 12214 | I.S. | 0.43764 | 12215 | KRAS | 0.43761 | 12216 | I.S. | 0.43757 |
| 12217 | I.S. | 0.43752 | 12218 | I.S. | 0.43752 | 12219 | I.S. | 0.43752 |
| 12220 | I.S. | 0.43751 | 12221 | I.S. | 0.4375 | 12222 | I.S. | 0.43748 |
| 12223 | I.S. | 0.43748 | 12224 | NOTCH2 | 0.43747 | 12225 | NF1 | 0.43746 |
| 12226 | I.S. | 0.43743 | 12227 | CBFB | 0.43739 | 12228 | PRKDC | 0.43737 |
| 12229 | I.S. | 0.43736 | 12230 | I.S. | 0.43736 | 12231 | SDHB | 0.43736 |
| 12232 | BCYRN1, | 0.43734 | 12233 | I.S. | 0.43725 | 12234 | I.S. | 0.43723 |
| | TAF1 | | | | | | | |
| 12235 | CSF1R | 0.43722 | 12236 | GLI1 | 0.4371 | 12237 | I.S. | 0.43708 |
| 12238 | EPHA5 | 0.43707 | 12239 | I.S. | 0.43706 | 12240 | I.S. | 0.43704 |
| 12241 | I.S. | 0.43701 | 12242 | I.S. | 0.43699 | 12243 | RANBP2 | 0.43698 |
| 12244 | PIK3CB | 0.43698 | 12245 | I.S. | 0.43698 | 12246 | I.S. | 0.43689 |
| 12247 | I.S. | 0.43687 | 12248 | I.S. | 0.43674 | 12249 | I.S. | 0.43669 |
| 12250 | AXIN2 | 0.43663 | 12251 | EP300 | 0.4366 | 12252 | PAK3 | 0.4366 |
| 12253 | PRKDC | 0.43657 | 12254 | EP300 | 0.43654 | 12255 | I.S. | 0.43651 |
| 12256 | KIT | 0.43651 | 12257 | ATRX | 0.43648 | 12258 | I.S. | 0.43647 |
| 12259 | I.S. | 0.43644 | 12260 | I.S. | 0.43644 | 12261 | ACVR1B | 0.43641 |
| 12262 | CUX1 | 0.43639 | 12263 | I.S. | 0.43636 | 12264 | I.S. | 0.4363 |
| 12265 | I.S. | 0.43627 | 12266 | CYLD | 0.43624 | 12267 | I.S. | 0.43624 |
| 12268 | KDM5C | 0.43619 | 12269 | I.S. | 0.43618 | 12270 | I.S. | 0.43616 |
| 12271 | I.S. | 0.43615 | 12272 | ATM | 0.43614 | 12273 | I.S. | 0.43609 |
| 12274 | I.S. | 0.43609 | 12275 | KDM6A | 0.43607 | 12276 | I.S. | 0.43607 |
| 12277 | I.S. | 0.43607 | 12278 | I.S. | 0.43603 | 12279 | I.S. | 0.43602 |
| 12280 | I.S. | 0.436 | 12281 | IL2RG | 0.43597 | 12282 | I.S. | 0.43593 |
| 12283 | I.S. | 0.43593 | 12284 | I.S. | 0.4359 | 12285 | I.S. | 0.4359 |
| 12286 | I.S. | 0.43584 | 12287 | I.S. | 0.43574 | 12288 | I.S. | 0.43573 |
| 12289 | FOXL2 | 0.4357 | 12290 | I.S. | 0.4357 | 12291 | STAG2 | 0.43565 |
| 12292 | I.S. | 0.43565 | 12293 | TRAF3 | 0.43564 | 12294 | DNMT3A | 0.43561 |
| 12295 | I.S. | 0.43558 | 12296 | PHF6 | 0.43556 | 12297 | I.S. | 0.43546 |
| 12298 | I.S. | 0.4354 | 12299 | I.S. | 0.43538 | 12300 | I.S. | 0.43532 |
| 12301 | I.S. | 0.43529 | 12302 | I.S. | 0.43523 | 12303 | I.S. | 0.43521 |
| 12304 | POLE | 0.4352 | 12305 | NOTCH1 | 0.43519 | 12306 | I.S. | 0.43517 |
| 12307 | I.S. | 0.43517 | 12308 | I.S. | 0.43517 | 12309 | I.S. | 0.43517 |
| 12310 | I.S. | 0.43516 | 12311 | MAGI2 | 0.43513 | 12312 | I.S. | 0.43509 |
| 12313 | I.S. | 0.43505 | 12314 | RTEL1 | 0.43502 | 12315 | I.S. | 0.43498 |
| 12316 | I.S. | 0.43493 | 12317 | I.S. | 0.4349 | 12318 | I.S. | 0.43484 |
| 12319 | FUBP1 | 0.43481 | 12320 | I.S. | 0.43479 | 12321 | I.S. | 0.43476 |
| 12322 | I.S. | 0.43475 | 12323 | RANBP2 | 0.43475 | 12324 | I.S. | 0.43473 |
| 12325 | I.S. | 0.43469 | 12326 | I.S. | 0.43467 | 12327 | ATR | 0.43464 |
| 12328 | I.S. | 0.43463 | 12329 | I.S. | 0.43458 | 12330 | I.S. | 0.43455 |
| 12331 | I.S. | 0.43454 | 12332 | I.S. | 0.43451 | 12333 | I.S. | 0.43451 |
| 12334 | ALK | 0.4345 | 12335 | I.S. | 0.43448 | 12336 | PIK3R1 | 0.43439 |
| 12337 | I.S. | 0.43439 | 12338 | PRKDC | 0.43433 | 12339 | I.S. | 0.43432 |
| 12340 | SETD2 | 0.43431 | 12341 | ARAF | 0.43428 | 12342 | PREX2 | 0.43425 |
| 12343 | EGFR | 0.43424 | 12344 | I.S. | 0.43424 | 12345 | I.S. | 0.43422 |
| 12346 | I.S. | 0.43422 | 12347 | I.S. | 0.43415 | 12348 | BAP1 | 0.43413 |
| 12349 | I.S. | 0.43413 | 12350 | I.S. | 0.43409 | 12351 | KDM6A | 0.43405 |
| 12352 | I.S. | 0.43404 | 12353 | FANCC | 0.43401 | 12354 | CUL3 | 0.43397 |
| 12355 | I.S. | 0.43397 | 12356 | I.S. | 0.43396 | 12357 | I.S. | 0.43392 |
| 12358 | I.S. | 0.43392 | 12359 | I.S. | 0.43391 | 12360 | SETD2 | 0.4339 |
| 12361 | RBM10 | 0.43387 | 12362 | GEN1 | 0.43386 | 12363 | I.S. | 0.43386 |
| 12364 | I.S. | 0.43385 | 12365 | I.S. | 0.43383 | 12366 | NOTCH2 | 0.43381 |
| 12367 | BAP1 | 0.4338 | 12368 | I.S. | 0.43377 | 12369 | I.S. | 0.43375 |
| 12370 | I.S. | 0.43366 | 12371 | GALNT12 | 0.43366 | 12372 | I.S. | 0.43365 |
| 12373 | I.S. | 0.43365 | 12374 | PAK3 | 0.43363 | 12375 | CREBBP | 0.43362 |
| 12376 | I.S. | 0.4336 | 12377 | MED12 | 0.4336 | 12378 | CXCR4 | 0.4336 |
| 12379 | I.S. | 0.43357 | 12380 | I.S. | 0.43351 | 12381 | I.S. | 0.43348 |
| 12382 | I.S. | 0.43348 | 12383 | PBRM1 | 0.43345 | 12384 | I.S. | 0.43344 |
| 12385 | INPP4B | 0.43342 | 12386 | I.S. | 0.43334 | 12387 | I.S. | 0.43332 |
| 12388 | WAS | 0.4333 | 12389 | I.S. | 0.43324 | 12390 | MAP2K4 | 0.43323 |
| 12391 | GATA1 | 0.43321 | 12392 | PIK3CG | 0.43318 | 12393 | ATRX | 0.43315 |
| 12394 | I.S. | 0.43315 | 12395 | I.S. | 0.43309 | 12396 | ERCC4 | 0.43309 |
| 12397 | I.S. | 0.43307 | 12398 | I.S. | 0.43306 | 12399 | AKT3 | 0.43303 |
| 12400 | I.S. | 0.43302 | 12401 | I.S. | 0.43299 | 12402 | CBLB | 0.43294 |
| 12403 | ATG2B | 0.43291 | 12404 | I.S. | 0.43287 | 12405 | BRAF | 0.43282 |
| 12406 | I.S. | 0.43282 | 12407 | I.S. | 0.43282 | 12408 | FANCC | 0.43281 |
| 12409 | I.S. | 0.4328 | 12410 | I.S. | 0.43277 | 12411 | I.S. | 0.43276 |
| 12412 | I.S. | 0.43271 | 12413 | SMAD2 | 0.43271 | 12414 | I.S. | 0.43267 |
| 12415 | TCIRG1 | 0.43267 | 12416 | I.S. | 0.43266 | 12417 | QKI | 0.43264 |
| 12418 | I.S. | 0.43262 | 12419 | I.S. | 0.43258 | 12420 | PIK3C2B | 0.43252 |
| 12421 | I.S. | 0.43252 | 12422 | I.S. | 0.4325 | 12423 | FUBP1 | 0.43247 |
| 12424 | I.S. | 0.43244 | 12425 | KDM5C | 0.43244 | 12426 | I.S. | 0.43244 |
| 12427 | CTC1 | 0.43244 | 12428 | GFI1B | 0.43241 | 12429 | ALK | 0.43241 |
| 12430 | I.S. | 0.43235 | 12431 | I.S. | 0.43232 | 12432 | I.S. | 0.43232 |
| 12433 | I.S. | 0.43232 | 12434 | I.S. | 0.4323 | 12435 | I.S. | 0.43229 |
| 12436 | I.S. | 0.43229 | 12437 | I.S. | 0.4322 | 12438 | NOTCH2 | 0.4322 |
| 12439 | I.S. | 0.43218 | 12440 | I.S. | 0.43217 | 12441 | I.S. | 0.43215 |
| 12442 | PDK1 | 0.43214 | 12443 | I.S. | 0.43214 | 12444 | PTEN | 0.43214 |
| 12445 | I.S. | 0.43213 | 12446 | I.S. | 0.43211 | 12447 | I.S. | 0.4321 |
| 12448 | I.S. | 0.43208 | 12449 | I.S. | 0.43208 | 12450 | I.S. | 0.43207 |
| 12451 | I.S. | 0.43202 | 12452 | I.S. | 0.432 | 12453 | I.S. | 0.43196 |
| 12454 | RANBP2 | 0.43195 | 12455 | I.S. | 0.43195 | 12456 | I.S. | 0.43184 |
| 12457 | I.S. | 0.43183 | 12458 | I.S. | 0.43181 | 12459 | GNAS | 0.43181 |
| 12460 | I.S. | 0.4318 | 12461 | MSH2 | 0.43179 | 12462 | I.S. | 0.43179 |
| 12463 | I.S. | 0.43169 | 12464 | SAMD9L | 0.43167 | 12465 | I.S. | 0.43167 |
| 12466 | I.S. | 0.43164 | 12467 | I.S. | 0.43163 | 12468 | EP300 | 0.43163 |
| 12469 | I.S. | 0.43159 | 12470 | I.S. | 0.43158 | 12471 | I.S. | 0.43157 |
| 12472 | RANBP2 | 0.43152 | 12473 | SPOP | 0.43146 | 12474 | I.S. | 0.43142 |
| 12475 | CDAN1 | 0.43141 | 12476 | ATR | 0.4314 | 12477 | DKC1 | 0.43137 |
| 12478 | NTRK1 | 0.43137 | 12479 | I.S. | 0.43134 | 12480 | TCF3 | 0.43134 |
| 12481 | I.S. | 0.43122 | 12482 | I.S. | 0.43121 | 12483 | MAP3K1 | 0.43118 |
| 12484 | I.S. | 0.43118 | 12485 | SLX4 | 0.43113 | 12486 | JESR1 | 0.4311 |
| 12487 | I.S. | 0.4311 | 12488 | I.S. | 0.4311 | 12489 | SMC1A | 0.43107 |
| 12490 | I.S. | 0.43106 | 12491 | I.S. | 0.43106 | 12492 | I.S. | 0.43106 |
| 12493 | I.S. | 0.43105 | 12494 | I.S. | 0.43104 | 12495 | I.S. | 0.43103 |
| 12496 | I.S. | 0.43102 | 12497 | I.S. | 0.43102 | 12498 | I.S. | 0.43101 |
| 12499 | I.S. | 0.43101 | 12500 | I.S. | 0.43101 | 12501 | GATA6 | 0.43101 |
| 12502 | I.S. | 0.43099 | 12503 | FH | 0.43098 | 12504 | I.S. | 0.43095 |
| 12505 | EP300 | 0.43093 | 12506 | I.S. | 0.43092 | 12507 | RB1 | 0.43089 |
| 12508 | I.S. | 0.43088 | 12509 | I.S. | 0.43086 | 12510 | MED12 | 0.43083 |
| 12511 | I.S. | 0.4308 | 12512 | I.S. | 0.4308 | 12513 | RPTOR | 0.4308 |
| 12514 | RNF43 | 0.43078 | 12515 | LRP1B | 0.43077 | 12516 | I.S. | 0.43077 |
| 12517 | I.S. | 0.43075 | 12518 | BCYRN1, | 0.43075 | 12519 | I.S. | 0.43073 |
| | | | | TAF1 | | | | |
| 12520 | FUBP1 | 0.43072 | 12521 | RAF1 | 0.43071 | 12522 | I.S. | 0.43068 |
| 12523 | KMT2D | 0.4306 | 12524 | I.S. | 0.4306 | 12525 | WAS | 0.43056 |
| 12526 | BCOR | 0.43053 | 12527 | MEN1 | 0.43051 | 12528 | DOT1L | 0.43048 |
| 12529 | I.S. | 0.43044 | 12530 | NF2 | 0.43044 | 12531 | I.S. | 0.43042 |
| 12532 | I.S. | 0.43036 | 12533 | EXO1 | 0.43035 | 12534 | I.S. | 0.43034 |
| 12535 | I.S. | 0.43033 | 12536 | I.S. | 0.43032 | 12537 | I.S. | 0.43031 |
| 12538 | I.S. | 0.4303 | 12539 | I.S. | 0.43027 | 12540 | I.S. | 0.43027 |
| 12541 | FUBP1 | 0.43024 | 12542 | I.S. | 0.43024 | 12543 | NFKBIA | 0.43021 |
| 12544 | MSH6 | 0.4302 | 12545 | I.S. | 0.43014 | 12546 | I.S. | 0.43012 |
| 12547 | BRAF | 0.43012 | 12548 | PALB2 | 0.4301 | 12549 | BCYRN1, | 0.43009 |
| | | | | | | | TAF1 | |
| 12550 | I.S. | 0.43009 | 12551 | AXIN2 | 0.43005 | 12552 | I.S. | 0.43001 |
| 12553 | I.S. | 0.42998 | 12554 | I.S. | 0.42998 | 12555 | DOT1L | 0.42994 |
| 12556 | I.S. | 0.42992 | 12557 | PDGFRA | 0.42983 | 12558 | I.S. | 0.42982 |
| 12559 | I.S. | 0.42979 | 12560 | I.S. | 0.42973 | 12561 | I.S. | 0.42968 |
| 12562 | I.S. | 0.42965 | 12563 | I.S. | 0.42961 | 12564 | CYLD | 0.42961 |
| 12565 | I.S. | 0.42961 | 12566 | I.S. | 0.42958 | 12567 | FUBP1 | 0.42956 |
| 12568 | I.S. | 0.42953 | 12569 | I.S. | 0.4295 | 12570 | I.S. | 0.42947 |
| 12571 | I.S. | 0.42947 | 12572 | I.S. | 0.42946 | 12573 | I.S. | 0.42944 |
| 12574 | PHF6 | 0.42941 | 12575 | I.S. | 0.4294 | 12576 | MSH6 | 0.4294 |
| 12577 | I.S. | 0.42938 | 12578 | I.S. | 0.42937 | 12579 | I.S. | 0.42936 |
| 12580 | RBM10 | 0.42935 | 12581 | I.S. | 0.42935 | 12582 | I.S. | 0.42932 |
| 12583 | I.S. | 0.42932 | 12584 | I.S. | 0.42932 | 12585 | I.S. | 0.42924 |
| 12586 | KRAS | 0.42924 | 12587 | I.S. | 0.4292 | 12588 | I.S. | 0.4292 |
| 12589 | I.S. | 0.42919 | 12590 | I.S. | 0.42917 | 12591 | KDM5C | 0.42914 |
| 12592 | I.S. | 0.42913 | 12593 | RBM10 | 0.4291 | 12594 | SMC1A | 0.4291 |
| 12595 | I.S. | 0.42908 | 12596 | I.S. | 0.42908 | 12597 | I.S. | 0.42908 |
| 12598 | I.S. | 0.42904 | 12599 | KDM6A | 0.42903 | 12600 | RBM10 | 0.42902 |
| 12601 | I.S. | 0.42901 | 12602 | KMT2D | 0.42901 | 12603 | I.S. | 0.42899 |
| 12604 | I.S. | 0.42898 | 12605 | CRLF2 | 0.42896 | 12606 | I.S. | 0.42896 |
| 12607 | I.S. | 0.42894 | 12608 | I.S. | 0.42894 | 12609 | BCR | 0.42894 |
| 12610 | I.S. | 0.42893 | 12611 | I.S. | 0.4289 | 12612 | I.S. | 0.42884 |
| 12613 | I.S. | 0.42883 | 12614 | RET | 0.42881 | 12615 | I.S. | 0.42881 |
| 12616 | MET | 0.42879 | 12617 | I.S. | 0.42878 | 12618 | I.S. | 0.42878 |
| 12619 | I.S. | 0.42876 | 12620 | I.S. | 0.42875 | 12621 | CTNNA1 | 0.42874 |
| 12622 | I.S. | 0.42872 | 12623 | I.S. | 0.42871 | 12624 | SMC3 | 0.42869 |
| 12625 | I.S. | 0.42867 | 12626 | ATR | 0.42863 | 12627 | I.S. | 0.42863 |
| 12628 | I.S. | 0.42863 | 12629 | I.S. | 0.42863 | 12630 | I.S. | 0.4286 |
| 12631 | I.S. | 0.4286 | 12632 | CUX1 | 0.42851 | 12633 | I.S. | 0.42851 |
| 12634 | I.S. | 0.42849 | 12635 | BCYRN1, | 0.42849 | 12636 | I.S. | 0.42842 |
| | | | | TAF1 | | | | |
| 12637 | I.S. | 0.42842 | 12638 | I.S. | 0.42842 | 12639 | I.S. | 0.42841 |
| 12640 | I.S. | 0.42837 | 12641 | I.S. | 0.42837 | 12642 | I.S. | 0.42837 |
| 12643 | I.S. | 0.42834 | 12644 | I.S. | 0.42833 | 12645 | I.S. | 0.42833 |
| 12646 | I.S. | 0.42831 | 12647 | DNM2 | 0.42826 | 12648 | I.S. | 0.42825 |
| 12649 | NOTCH3 | 0.42825 | 12650 | I.S. | 0.42822 | 12651 | I.S. | 0.42822 |
| 12652 | I.S. | 0.42819 | 12653 | I.S. | 0.42817 | 12654 | AURKC | 0.42816 |
| 12655 | I.S. | 0.42813 | 12656 | POT1 | 0.42813 | 12657 | I.S. | 0.4281 |
| 12658 | I.S. | 0.42809 | 12659 | KMT2A | 0.42805 | 12660 | I.S. | 0.42804 |
| 12661 | I.S. | 0.42803 | 12662 | MVK | 0.42801 | 12663 | I.S. | 0.42801 |
| 12664 | I.S. | 0.42801 | 12665 | I.S. | 0.42801 | 12666 | I.S. | 0.42795 |
| 12667 | I.S. | 0.42795 | 12668 | I.S. | 0.42792 | 12669 | I.S. | 0.42791 |
| 12670 | EPCAM | 0.4279 | 12671 | PIK3CG | 0.42788 | 12672 | I.S. | 0.42788 |
| 12673 | I.S. | 0.42786 | 12674 | EP300 | 0.42786 | 12675 | ARFRP1 | 0.42785 |
| 12676 | BCYRN1, | 0.42784 | 12677 | I.S. | 0.42781 | 12678 | FGFR3 | 0.4278 |
| | TAF1 | | | | | | | |
| 12679 | I.S. | 0.4278 | 12680 | PRKDC | 0.42777 | 12681 | I.S. | 0.42775 |
| 12682 | GFI1 | 0.42774 | 12683 | I.S. | 0.42772 | 12684 | FUBP1 | 0.42771 |
| 12685 | I.S. | 0.42769 | 12686 | I.S. | 0.42769 | 12687 | I.S. | 0.42763 |
| 12688 | I.S. | 0.42763 | 12689 | PRKDC | 0.42762 | 12690 | I.S. | 0.42759 |
| 12691 | I.S. | 0.42759 | 12692 | MVK | 0.42757 | 12693 | I.S. | 0.42756 |
| 12694 | I.S. | 0.42756 | 12695 | I.S. | 0.42756 | 12696 | I.S. | 0.42753 |
| 12697 | I.S. | 0.42751 | 12698 | I.S. | 0.42749 | 12699 | I.S. | 0.42748 |
| 12700 | I.S. | 0.42739 | 12701 | I.S. | 0.42735 | 12702 | I.S. | 0.42735 |
| 12703 | I.S. | 0.42729 | 12704 | I.S. | 0.42727 | 12705 | I.S. | 0.42723 |
| 12706 | SYK | 0.42722 | 12707 | I.S. | 0.42722 | 12708 | I.S. | 0.42718 |
| 12709 | — | 0.42714 | 12710 | I.S. | 0.42708 | 12711 | I.S. | 0.42707 |
| 12712 | I.S. | 0.42703 | 12713 | I.S. | 0.42698 | 12714 | I.S. | 0.42697 |
| 12715 | I.S. | 0.42694 | 12716 | I.S. | 0.42694 | 12717 | RBM10 | 0.42691 |
| 12718 | I.S. | 0.42691 | 12719 | I.S. | 0.42691 | 12720 | I.S. | 0.42691 |
| 12721 | I.S. | 0.4269 | 12722 | I.S. | 0.42682 | 12723 | I.S. | 0.42682 |
| 12724 | KMT2D | 0.42679 | 12725 | NSD2 | 0.42677 | 12726 | FANCD2 | 0.42677 |
| 12727 | I.S. | 0.42677 | 12728 | TP53 | 0.42677 | 12729 | I.S. | 0.42673 |
| 12730 | I.S. | 0.42673 | 12731 | AURKA | 0.4267 | 12732 | I.S. | 0.42667 |
| 12733 | I.S. | 0.42665 | 12734 | I.S. | 0.42664 | 12735 | I.S. | 0.42664 |
| 12736 | I.S. | 0.42661 | 12737 | I.S. | 0.42661 | 12738 | I.S. | 0.42661 |
| 12739 | I.S. | 0.42661 | 12740 | I.S. | 0.4266 | 12741 | I.S. | 0.42659 |
| 12742 | I.S. | 0.42659 | 12743 | NPM1 | 0.42658 | 12744 | I.S. | 0.42658 |
| 12745 | I.S. | 0.42658 | 12746 | I.S. | 0.42656 | 12747 | PRKDC | 0.42653 |
| 12748 | IL2RG | 0.4265 | 12749 | I.S. | 0.4265 | 12750 | ATG2B | 0.4265 |
| 12751 | SPOP | 0.4265 | 12752 | KIT | 0.42647 | 12753 | I.S. | 0.42646 |
| 12754 | I.S. | 0.42644 | 12755 | I.S. | 0.42644 | 12756 | I.S. | 0.42641 |
| 12757 | I.S. | 0.42641 | 12758 | I.S. | 0.42641 | 12759 | I.S. | 0.42641 |
| 12760 | I.S. | 0.4264 | 12761 | I.S. | 0.4264 | 12762 | KDM6A | 0.42638 |
| 12763 | I.S. | 0.42636 | 12764 | I.S. | 0.42628 | 12765 | I.S. | 0.42622 |
| 12766 | PRKDC | 0.4262 | 12767 | I.S. | 0.42618 | 12768 | I.S. | 0.42617 |
| 12769 | I.S. | 0.42617 | 12770 | BCYRN1, | 0.42617 | 12771 | DNM2 | 0.42614 |
| | | | | TAF1 | | | | |
| 12772 | RBM10 | 0.42614 | 12773 | I.S. | 0.42611 | 12774 | I.S. | 0.42609 |
| 12775 | I.S. | 0.42608 | 12776 | I.S. | 0.42606 | 12777 | I.S. | 0.42605 |
| 12778 | CBLB | 0.42602 | 12779 | FUBP1 | 0.42599 | 12780 | EPHA5 | 0.42596 |
| 12781 | SUFU | 0.42596 | 12782 | I.S. | 0.4259 | 12783 | I.S. | 0.42588 |
| 12784 | WAS | 0.42585 | 12785 | I.S. | 0.42584 | 12786 | I.S. | 0.42583 |
| 12787 | I.S. | 0.42582 | 12788 | I.S. | 0.42581 | 12789 | MED12 | 0.42581 |
| 12790 | I.S. | 0.42578 | 12791 | I.S. | 0.42578 | 12792 | I.S. | 0.42575 |
| 12793 | I.S. | 0.42575 | 12794 | I.S. | 0.42575 | 12795 | I.S. | 0.42573 |
| 12796 | I.S. | 0.42573 | 12797 | I.S. | 0.4257 | 12798 | I.S. | 0.4257 |
| 12799 | I.S. | 0.42569 | 12800 | HOXB13 | 0.42565 | 12801 | I.S. | 0.42563 |
| 12802 | I.S. | 0.4256 | 12803 | NOTCH1 | 0.42557 | 12804 | I.S. | 0.42555 |
| 12805 | I.S. | 0.42548 | 12806 | I.S. | 0.42546 | 12807 | I.S. | 0.42543 |
| 12808 | I.S. | 0.42542 | 12809 | I.S. | 0.42541 | 12810 | KDM5C | 0.4254 |
| 12811 | MSH2 | 0.42537 | 12812 | I.S. | 0.4253 | 12813 | I.S. | 0.4253 |
| 12814 | CDAN1 | 0.42529 | 12815 | HNF1A | 0.42527 | 12816 | I.S. | 0.42519 |
| 12817 | RANBP2 | 0.42516 | 12818 | I.S. | 0.42516 | 12819 | I.S. | 0.42513 |
| 12820 | I.S. | 0.42512 | 12821 | KMT2D | 0.42508 | 12822 | I.S. | 0.42508 |
| 12823 | STAG2 | 0.42508 | 12824 | BCORL1 | 0.42507 | 12825 | I.S. | 0.42506 |
| 12826 | FANCI | 0.42505 | 12827 | I.S. | 0.42504 | 12828 | I.S. | 0.42504 |
| 12829 | I.S. | 0.42504 | 12830 | EZH2 | 0.42504 | 12831 | I.S. | 0.42503 |
| 12832 | SDHD | 0.42501 | 12833 | MDM4 | 0.42501 | 12834 | IL2RG | 0.42499 |
| 12835 | I.S. | 0.42498 | 12836 | I.S. | 0.42498 | 12837 | I.S. | 0.42492 |
| 12838 | I.S. | 0.4249 | 12839 | I.S. | 0.42489 | 12840 | I.S. | 0.42486 |
| 12841 | I.S. | 0.42484 | 12842 | I.S. | 0.42483 | 12843 | I.S. | 0.42483 |
| 12844 | I.S. | 0.42477 | 12845 | I.S. | 0.42477 | 12846 | I.S. | 0.4247 |
| 12847 | I.S. | 0.42468 | 12848 | CUL3 | 0.42468 | 12849 | I.S. | 0.42462 |
| 12850 | I.S. | 0.42459 | 12851 | I.S. | 0.42459 | 12852 | I.S. | 0.42459 |
| 12853 | I.S. | 0.42456 | 12854 | I.S. | 0.42456 | 12855 | I.S. | 0.4245 |
| 12856 | GID4 | 0.42449 | 12857 | ATM | 0.42447 | 12858 | I.S. | 0.42442 |
| 12859 | I.S. | 0.42439 | 12860 | PTCH1 | 0.42439 | 12861 | ANKRD26 | 0.42439 |
| 12862 | I.S. | 0.42439 | 12863 | ATG2B | 0.42434 | 12864 | I.S. | 0.42427 |
| 12865 | ERBB4 | 0.42427 | 12866 | SMC3 | 0.42427 | 12867 | I.S. | 0.42423 |
| 12868 | I.S. | 0.42421 | 12869 | I.S. | 0.42416 | 12870 | MED12 | 0.42415 |
| 12871 | BARD1 | 0.42414 | 12872 | I.S. | 0.42414 | 12873 | I.S. | 0.42409 |
| 12874 | I.S. | 0.42409 | 12875 | I.S. | 0.42406 | 12876 | I.S. | 0.42406 |
| 12877 | I.S. | 0.42404 | 12878 | I.S. | 0.42403 | 12879 | I.S. | 0.42402 |
| 12880 | I.S. | 0.42401 | 12881 | I.S. | 0.42401 | 12882 | KDR | 0.424 |
| 12883 | I.S. | 0.42399 | 12884 | FOXL2 | 0.42398 | 12885 | CSF1R | 0.42397 |
| 12886 | I.S. | 0.42394 | 12887 | SMAD3 | 0.42393 | 12888 | I.S. | 0.42392 |
| 12889 | I.S. | 0.42391 | 12890 | I.S. | 0.4239 | 12891 | I.S. | 0.42385 |
| 12892 | I.S. | 0.42385 | 12893 | ELANE | 0.42385 | 12894 | I.S. | 0.42383 |
| 12895 | RUNX1T1 | 0.42382 | 12896 | I.S. | 0.42379 | 12897 | I.S. | 0.42377 |
| 12898 | I.S. | 0.42377 | 12899 | I.S. | 0.42376 | 12900 | I.S. | 0.42376 |
| 12901 | I.S. | 0.42376 | 12902 | I.S. | 0.42365 | 12903 | I.S. | 0.42365 |
| 12904 | I.S. | 0.4236 | 12905 | I.S. | 0.4236 | 12906 | I.S. | 0.42358 |
| 12907 | I.S. | 0.42357 | 12908 | I.S. | 0.42356 | 12909 | I.S. | 0.42353 |
| 12910 | I.S. | 0.42353 | 12911 | I.S. | 0.42353 | 12912 | I.S. | 0.42352 |
| 12913 | TOP2A | 0.42343 | 12914 | BRCA1 | 0.42341 | 12915 | I.S. | 0.42338 |
| 12916 | I.S. | 0.42337 | 12917 | MTOR | 0.42332 | 12918 | RNF43 | 0.42326 |
| 12919 | I.S. | 0.42326 | 12920 | NOTCH3 | 0.42326 | 12921 | I.S. | 0.42323 |
| 12922 | I.S. | 0.4232 | 12923 | I.S. | 0.42319 | 12924 | I.S. | 0.42319 |
| 12925 | I.S. | 0.42314 | 12926 | DOT1L | 0.42314 | 12927 | I.S. | 0.42313 |
| 12928 | SLX4 | 0.42313 | 12929 | I.S. | 0.4231 | 12930 | ROS1 | 0.42309 |
| 12931 | I.S. | 0.42309 | 12932 | I.S. | 0.42307 | 12933 | PRKDC | 0.42306 |
| 12934 | MTOR | 0.42299 | 12935 | I.S. | 0.42299 | 12936 | I.S. | 0.42299 |
| 12937 | I.S. | 0.42298 | 12938 | FGF4 | 0.42296 | 12939 | I.S. | 0.42294 |
| 12940 | BTK | 0.42293 | 12941 | I.S. | 0.42291 | 12942 | I.S. | 0.4229 |
| 12943 | EMSY | 0.42288 | 12944 | ERCC4 | 0.42288 | 12945 | I.S. | 0.42287 |
| 12946 | CDH1 | 0.42287 | 12947 | I.S. | 0.42283 | 12948 | I.S. | 0.42279 |
| 12949 | I.S. | 0.42277 | 12950 | I.S. | 0.42275 | 12951 | I.S. | 0.42272 |
| 12952 | I.S. | 0.42272 | 12953 | I.S. | 0.42269 | 12954 | I.S. | 0.42269 |
| 12955 | I.S. | 0.42269 | 12956 | I.S. | 0.42269 | 12957 | I.S. | 0.42267 |
| 12958 | I.S. | 0.42266 | 12959 | SLX4 | 0.42266 | 12960 | I.S. | 0.42263 |
| 12961 | I.S. | 0.42261 | 12962 | I.S. | 0.42261 | 12963 | I.S. | 0.42259 |
| 12964 | I.S. | 0.42258 | 12965 | I.S. | 0.42257 | 12966 | I.S. | 0.42257 |
| 12967 | I.S. | 0.42254 | 12968 | I.S. | 0.42254 | 12969 | I.S. | 0.42249 |
| 12970 | SDHA | 0.42249 | 12971 | I.S. | 0.42248 | 12972 | LRP1B | 0.42245 |
| 12973 | KEAP1 | 0.42245 | 12974 | ALK | 0.42239 | 12975 | I.S. | 0.42237 |
| 12976 | I.S. | 0.42237 | 12977 | I.S. | 0.42237 | 12978 | I.S. | 0.42236 |
| 12979 | I.S. | 0.42234 | 12980 | I.S. | 0.42233 | 12981 | I.S. | 0.42231 |
| 12982 | I.S. | 0.4223 | 12983 | I.S. | 0.42228 | 12984 | I.S. | 0.42228 |
| 12985 | I.S. | 0.42227 | 12986 | I.S. | 0.42225 | 12987 | I.S. | 0.42225 |
| 12988 | I.S. | 0.42224 | 12989 | I.S. | 0.42222 | 12990 | I.S. | 0.42219 |
| 12991 | I.S. | 0.42218 | 12992 | I.S. | 0.42215 | 12993 | FANCI | 0.42215 |
| 12994 | I.S. | 0.42212 | 12995 | CUL3 | 0.42212 | 12996 | I.S. | 0.42207 |
| 12997 | PTCH1 | 0.42207 | 12998 | I.S. | 0.42204 | 12999 | EMSY | 0.42204 |
| 13000 | I.S. | 0.42204 | 13001 | I.S. | 0.42204 | 13002 | I.S. | 0.42201 |
| 13003 | I.S. | 0.42201 | 13004 | I.S. | 0.42198 | 13005 | I.S. | 0.42196 |
| 13006 | NOTCH3 | 0.42196 | 13007 | I.S. | 0.42195 | 13008 | I.S. | 0.42195 |
| 13009 | I.S. | 0.42192 | 13010 | I.S. | 0.42192 | 13011 | FANCD2 | 0.4219 |
| 13012 | I.S. | 0.42189 | 13013 | BTK | 0.42187 | 13014 | I.S. | 0.42186 |
| 13015 | SPEN | 0.42186 | 13016 | I.S. | 0.42186 | 13017 | GATA2 | 0.42185 |
| 13018 | BMPR1A | 0.42184 | 13019 | I.S. | 0.42177 | 13020 | PRKDC | 0.42175 |
| 13021 | I.S. | 0.42174 | 13022 | I.S. | 0.42174 | 13023 | I.S. | 0.42168 |
| 13024 | RPTOR | 0.42166 | 13025 | AR | 0.42166 | 13026 | BCYRN1, | 0.42166 |
| | | | | | | | TAF1 | |
| 13027 | RANBP2 | 0.42163 | 13028 | STAT3 | 0.42163 | 13029 | I.S. | 0.42162 |
| 13030 | I.S. | 0.4216 | 13031 | BAP1 | 0.4216 | 13032 | I.S. | 0.42159 |
| 13033 | I.S. | 0.42154 | 13034 | AURKB | 0.4215 | 13035 | ANKRD26 | 0.42148 |
| 13036 | I.S. | 0.42138 | 13037 | CTNNA1 | 0.42138 | 13038 | I.S. | 0.42135 |
| 13039 | BRAF | 0.4213 | 13040 | I.S. | 0.4213 | 13041 | ERRFI1 | 0.4213 |
| 13042 | I.S. | 0.42127 | 13043 | PTPN11 | 0.42127 | 13044 | RHEB | 0.42127 |
| 13045 | ATRX | 0.42121 | 13046 | I.S. | 0.42121 | 13047 | I.S. | 0.42121 |
| 13048 | I.S. | 0.4212 | 13049 | I.S. | 0.42117 | 13050 | I.S. | 0.42117 |
| 13051 | I.S. | 0.42114 | 13052 | I.S. | 0.42114 | 13053 | I.S. | 0.42109 |
| 13054 | FLT4 | 0.42109 | 13055 | I.S. | 0.42109 | 13056 | I.S. | 0.42109 |
| 13057 | I.S. | 0.42108 | 13058 | I.S. | 0.42106 | 13059 | I.S. | 0.42105 |
| 13060 | I.S. | 0.42104 | 13061 | I.S. | 0.421 | 13062 | I.S. | 0.421 |
| 13063 | I.S. | 0.421 | 13064 | I.S. | 0.421 | 13065 | I.S. | 0.42097 |
| 13066 | I.S. | 0.42093 | 13067 | EZH2 | 0.42092 | 13068 | I.S. | 0.42088 |
| 13069 | I.S. | 0.42084 | 13070 | I.S. | 0.42082 | 13071 | I.S. | 0.4208 |
| 13072 | I.S. | 0.42079 | 13073 | KRAS | 0.42077 | 13074 | I.S. | 0.42076 |
| 13075 | I.S. | 0.42076 | 13076 | I.S. | 0.42074 | 13077 | I.S. | 0.4207 |
| 13078 | I.S. | 0.42068 | 13079 | I.S. | 0.42064 | 13080 | I.S. | 0.42063 |
| 13081 | MUTYH, | 0.42061 | 13082 | I.S. | 0.42058 | 13083 | I.S. | 0.42056 |
| | TOE1 | | | | | | | |
| 13084 | I.S. | 0.42056 | 13085 | I.S. | 0.42053 | 13086 | I.S. | 0.42052 |
| 13087 | I.S. | 0.42051 | 13088 | I.S. | 0.42045 | 13089 | PALB2 | 0.42043 |
| 13090 | BCOR | 0.42037 | 13091 | NBN | 0.42036 | 13092 | I.S. | 0.42035 |
| 13093 | EP300 | 0.42032 | 13094 | I.S. | 0.42032 | 13095 | I.S. | 0.42029 |
| 13096 | I.S. | 0.42028 | 13097 | I.S. | 0.42028 | 13098 | I.S. | 0.42027 |
| 13099 | I.S. | 0.42026 | 13100 | I.S. | 0.42023 | 13101 | I.S. | 0.42021 |
| 13102 | I.S. | 0.4202 | 13103 | RNF43 | 0.4202 | 13104 | I.S. | 0.4202 |
| 13105 | BLM | 0.42018 | 13106 | KDM6A | 0.42017 | 13107 | I.S. | 0.42017 |
| 13108 | KDR | 0.42016 | 13109 | I.S. | 0.42014 | 13110 | I.S. | 0.42014 |
| 13111 | I.S. | 0.42014 | 13112 | MITF | 0.42012 | 13113 | I.S. | 0.42011 |
| 13114 | ABCB7 | 0.42008 | 13115 | I.S. | 0.42002 | 13116 | I.S. | 0.41999 |
| 13117 | I.S. | 0.41999 | 13118 | I.S. | 0.41996 | 13119 | BRCA1 | 0.41995 |
| 13120 | I.S. | 0.41995 | 13121 | I.S. | 0.41995 | 13122 | I.S. | 0.41994 |
| 13123 | MAPK1 | 0.41994 | 13124 | I.S. | 0.41993 | 13125 | I.S. | 0.41993 |
| 13126 | RANBP2 | 0.4199 | 13127 | I.S. | 0.4199 | 13128 | PRKDC | 0.41988 |
| 13129 | I.S. | 0.41987 | 13130 | I.S. | 0.41985 | 13131 | PBRM1 | 0.41984 |
| 13132 | I.S. | 0.41982 | 13133 | I.S. | 0.4198 | 13134 | I.S. | 0.41979 |
| 13135 | I.S. | 0.41978 | 13136 | I.S. | 0.41978 | 13137 | I.S. | 0.41975 |
| 13138 | I.S. | 0.41975 | 13139 | I.S. | 0.41974 | 13140 | I.S. | 0.41973 |
| 13141 | TCF3 | 0.41969 | 13142 | I.S. | 0.41968 | 13143 | PALB2 | 0.41967 |
| 13144 | KMT2D | 0.41966 | 13145 | I.S. | 0.41965 | 13146 | I.S. | 0.41964 |
| 13147 | I.S. | 0.41963 | 13148 | I.S. | 0.4196 | 13149 | I.S. | 0.4196 |
| 13150 | I.S. | 0.41958 | 13151 | PTPN11 | 0.41954 | 13152 | I.S. | 0.41954 |
| 13153 | I.S. | 0.41954 | 13154 | I.S. | 0.41949 | 13155 | I.S. | 0.41946 |
| 13156 | I.S. | 0.41946 | 13157 | I.S. | 0.41946 | 13158 | I.S. | 0.41936 |
| 13159 | I.S. | 0.41936 | 13160 | I.S. | 0.41936 | 13161 | I.S. | 0.41936 |
| 13162 | I.S. | 0.41933 | 13163 | MEFV | 0.4193 | 13164 | I.S. | 0.4193 |
| 13165 | I.S. | 0.41925 | 13166 | I.S. | 0.41925 | 13167 | I.S. | 0.41922 |
| 13168 | I.S. | 0.41921 | 13169 | I.S. | 0.4192 | 13170 | I.S. | 0.41919 |
| 13171 | I.S. | 0.41919 | 13172 | I.S. | 0.41919 | 13173 | I.S. | 0.41919 |
| 13174 | I.S. | 0.41917 | 13175 | XPO1 | 0.41916 | 13176 | I.S. | 0.41916 |
| 13177 | I.S. | 0.41913 | 13178 | DKC1 | 0.41911 | 13179 | I.S. | 0.4191 |
| 13180 | I.S. | 0.41909 | 13181 | I.S. | 0.41907 | 13182 | I.S. | 0.41907 |
| 13183 | MTOR | 0.41907 | 13184 | I.S. | 0.41907 | 13185 | I.S. | 0.41901 |
| 13186 | CHD2 | 0.41899 | 13187 | I.S. | 0.41897 | 13188 | — | 0.41896 |
| 13189 | RB1 | 0.41895 | 13190 | MTOR | 0.41895 | 13191 | TCF3 | 0.41894 |
| 13192 | I.S. | 0.41892 | 13193 | I.S. | 0.41889 | 13194 | STAG2 | 0.41887 |
| 13195 | I.S. | 0.41886 | 13196 | I.S. | 0.41884 | 13197 | I.S. | 0.4188 |
| 13198 | I.S. | 0.41875 | 13199 | I.S. | 0.41875 | 13200 | I.S. | 0.41874 |
| 13201 | I.S. | 0.41874 | 13202 | I.S. | 0.4187 | 13203 | INPP4B | 0.41868 |
| 13204 | I.S. | 0.41868 | 13205 | BCR | 0.41868 | 13206 | BRD4 | 0.41865 |
| 13207 | I.S. | 0.41865 | 13208 | I.S. | 0.41863 | 13209 | KMT2B | 0.41862 |
| 13210 | I.S. | 0.41859 | 13211 | I.S. | 0.41859 | 13212 | I.S. | 0.41859 |
| 13213 | I.S. | 0.41855 | 13214 | I.S. | 0.41854 | 13215 | LRP1B | 0.41854 |
| 13216 | CHD2 | 0.41851 | 13217 | AXIN2 | 0.4185 | 13218 | CDKN2C | 0.4185 |
| 13219 | I.S. | 0.4185 | 13220 | KMT2B | 0.41848 | 13221 | I.S. | 0.41847 |
| 13222 | GNA11 | 0.41842 | 13223 | BCR | 0.41841 | 13224 | I.S. | 0.41839 |
| 13225 | I.S. | 0.41839 | 13226 | I.S. | 0.41838 | 13227 | PMS2 | 0.41838 |
| 13228 | I.S. | 0.41829 | 13229 | DDX11 | 0.41829 | 13230 | GRM3 | 0.41827 |
| 13231 | I.S. | 0.41826 | 13232 | I.S. | 0.41821 | 13233 | RICTOR | 0.41818 |
| 13234 | I.S. | 0.41818 | 13235 | SMARCA4 | 0.41817 | 13236 | I.S. | 0.41815 |
| 13237 | I.S. | 0.41813 | 13238 | I.S. | 0.41809 | 13239 | I.S. | 0.41809 |
| 13240 | I.S. | 0.41803 | 13241 | I.S. | 0.418 | 13242 | I.S. | 0.41797 |
| 13243 | KDM6A | 0.41791 | 13244 | I.S. | 0.41789 | 13245 | I.S. | 0.41788 |
| 13246 | CHD2 | 0.41788 | 13247 | I.S. | 0.41788 | 13248 | I.S. | 0.41786 |
| 13249 | GREM1 | 0.41786 | 13250 | I.S. | 0.41785 | 13251 | I.S. | 0.41782 |
| 13252 | I.S. | 0.41782 | 13253 | FANCE | 0.41777 | 13254 | RAD51 | 0.41777 |
| 13255 | I.S. | 0.41773 | 13256 | ANKRD26 | 0.41772 | 13257 | I.S. | 0.41769 |
| 13258 | I.S. | 0.41768 | 13259 | I.S. | 0.41768 | 13260 | I.S. | 0.41767 |
| 13261 | PRKDC | 0.41765 | 13262 | POT1 | 0.41764 | 13263 | I.S. | 0.41763 |
| 13264 | I.S. | 0.41762 | 13265 | I.S. | 0.41761 | 13266 | I.S. | 0.41759 |
| 13267 | I.S. | 0.41758 | 13268 | NUP93 | 0.4175 | 13269 | I.S. | 0.41747 |
| 13270 | I.S. | 0.41747 | 13271 | I.S. | 0.41747 | 13272 | SPEN | 0.41746 |
| 13273 | I.S. | 0.41744 | 13274 | I.S. | 0.41738 | 13275 | I.S. | 0.41735 |
| 13276 | I.S. | 0.41735 | 13277 | RARA | 0.41735 | 13278 | I.S. | 0.41734 |
| 13279 | I.S. | 0.41732 | 13280 | I.S. | 0.41729 | 13281 | I.S. | 0.41728 |
| 13282 | I.S. | 0.41728 | 13283 | I.S. | 0.41726 | 13284 | SBDS | 0.41726 |
| 13285 | I.S. | 0.41723 | 13286 | I.S. | 0.41722 | 13287 | I.S. | 0.41721 |
| 13288 | I.S. | 0.41719 | 13289 | I.S. | 0.41719 | 13290 | BCOR | 0.41717 |
| 13291 | BRD4 | 0.41714 | 13292 | I.S. | 0.41714 | 13293 | I.S. | 0.41714 |
| 13294 | ETV6 | 0.41714 | 13295 | RAD50 | 0.41712 | 13296 | I.S. | 0.41711 |
| 13297 | I.S. | 0.41707 | 13298 | I.S. | 0.41703 | 13299 | I.S. | 0.41703 |
| 13300 | I.S. | 0.41703 | 13301 | I.S. | 0.417 | 13302 | RBM10 | 0.41697 |
| 13303 | I.S. | 0.41694 | 13304 | I.S. | 0.4169 | 13305 | KMT2A | 0.4169 |
| 13306 | I.S. | 0.41689 | 13307 | TERT | 0.41689 | 13308 | I.S. | 0.41686 |
| 13309 | NOTCH1 | 0.41684 | 13310 | ARID2 | 0.41679 | 13311 | I.S. | 0.41678 |
| 13312 | I.S. | 0.41678 | 13313 | I.S. | 0.41678 | 13314 | I.S. | 0.41676 |
| 13315 | HGF | 0.41675 | 13316 | I.S. | 0.4167 | 13317 | I.S. | 0.41668 |
| 13318 | I.S. | 0.41666 | 13319 | I.S. | 0.41666 | 13320 | BCOR | 0.41664 |
| 13321 | I.S. | 0.41664 | 13322 | I.S. | 0.4166 | 13323 | I.S. | 0.41657 |
| 13324 | I.S. | 0.41655 | 13325 | I.S. | 0.41654 | 13326 | I.S. | 0.41652 |
| 13327 | LRP1B | 0.41648 | 13328 | I.S. | 0.41648 | 13329 | I.S. | 0.41645 |
| 13330 | I.S. | 0.41643 | 13331 | I.S. | 0.41637 | 13332 | EPAS1 | 0.41636 |
| 13333 | I.S. | 0.41634 | 13334 | I.S. | 0.41633 | 13335 | I.S. | 0.41633 |
| 13336 | I.S. | 0.41628 | 13337 | I.S. | 0.41627 | 13338 | PAX5 | 0.41624 |
| 13339 | I.S. | 0.41623 | 13340 | BCL6 | 0.41621 | 13341 | I.S. | 0.41612 |
| 13342 | I.S. | 0.41611 | 13343 | EP300 | 0.41611 | 13344 | KMT2B | 0.4161 |
| 13345 | BCYRN1, | 0.4161 | 13346 | I.S. | 0.41609 | 13347 | I.S. | 0.41607 |
| | TAF1 | | | | | | | |
| 13348 | NF1 | 0.41605 | 13349 | I.S. | 0.41604 | 13350 | I.S. | 0.41604 |
| 13351 | I.S. | 0.41604 | 13352 | I.S. | 0.41602 | 13353 | I.S. | 0.41599 |
| 13354 | I.S. | 0.41596 | 13355 | I.S. | 0.41592 | 13356 | JAPC | 0.41591 |
| 13357 | SAMD9L | 0.41586 | 13358 | I.S. | 0.41586 | 13359 | I.S. | 0.41586 |
| 13360 | I.S. | 0.41583 | 13361 | I.S. | 0.41583 | 13362 | I.S. | 0.41583 |
| 13363 | NOP10 | 0.41583 | 13364 | ANKRD26 | 0.41581 | 13365 | PRKDC | 0.4158 |
| 13366 | LRP1B | 0.41577 | 13367 | I.S. | 0.41577 | 13368 | I.S. | 0.41575 |
| 13369 | I.S. | 0.41575 | 13370 | PIK3R1 | 0.41575 | 13371 | TERF1 | 0.41575 |
| 13372 | NOTCH2 | 0.41574 | 13373 | I.S. | 0.41574 | 13374 | I.S. | 0.41572 |
| 13375 | I.S. | 0.41571 | 13376 | I.S. | 0.4157 | 13377 | NUP93 | 0.4157 |
| 13378 | I.S. | 0.41568 | 13379 | KMT2D | 0.41568 | 13380 | I.S. | 0.41567 |
| 13381 | I.S. | 0.41566 | 13382 | I.S. | 0.41563 | 13383 | I.S. | 0.41562 |
| 13384 | I.S. | 0.41562 | 13385 | I.S. | 0.41562 | 13386 | I.S. | 0.41561 |
| 13387 | I.S. | 0.41557 | 13388 | MAPK1 | 0.41557 | 13389 | I.S. | 0.41556 |
| 13390 | MAGI2 | 0.41555 | 13391 | I.S. | 0.4155 | 13392 | NBN | 0.41548 |
| 13393 | I.S. | 0.41548 | 13394 | I.S. | 0.41547 | 13395 | I.S. | 0.41545 |
| 13396 | I.S. | 0.41543 | 13397 | I.S. | 0.41543 | 13398 | I.S. | 0.41541 |
| 13399 | I.S. | 0.41541 | 13400 | I.S. | 0.41539 | 13401 | I.S. | 0.41536 |
| 13402 | I.S. | 0.41535 | 13403 | I.S. | 0.41533 | 13404 | I.S. | 0.41533 |
| 13405 | I.S. | 0.41532 | 13406 | I.S. | 0.41532 | 13407 | I.S. | 0.4153 |
| 13408 | I.S. | 0.41529 | 13409 | I.S. | 0.41526 | 13410 | I.S. | 0.41523 |
| 13411 | I.S. | 0.4152 | 13412 | JUN | 0.41517 | 13413 | I.S. | 0.41517 |
| 13414 | I.S. | 0.41514 | 13415 | I.S. | 0.41512 | 13416 | I.S. | 0.4151 |
| 13417 | I.S. | 0.4151 | 13418 | I.S. | 0.41509 | 13419 | — | 0.41509 |
| 13420 | I.S. | 0.41509 | 13421 | I.S. | 0.41503 | 13422 | I.S. | 0.41503 |
| 13423 | I.S. | 0.415 | 13424 | I.S. | 0.415 | 13425 | I.S. | 0.415 |
| 13426 | I.S. | 0.41497 | 13427 | DDX11 | 0.41496 | 13428 | I.S. | 0.41491 |
| 13429 | I.S. | 0.41491 | 13430 | I.S. | 0.4149 | 13431 | I.S. | 0.4149 |
| 13432 | I.S. | 0.4149 | 13433 | I.S. | 0.41489 | 13434 | I.S. | 0.41489 |
| 13435 | I.S. | 0.41487 | 13436 | I.S. | 0.41484 | 13437 | I.S. | 0.41484 |
| 13438 | I.S. | 0.41478 | 13439 | ANKRD26 | 0.41476 | 13440 | I.S. | 0.41476 |
| 13441 | I.S. | 0.41473 | 13442 | NOTCH1 | 0.41471 | 13443 | I.S. | 0.4147 |
| 13444 | I.S. | 0.41468 | 13445 | I.S. | 0.41468 | 13446 | I.S. | 0.41467 |
| 13447 | I.S. | 0.41466 | 13448 | BCORL1 | 0.41463 | 13449 | I.S. | 0.41461 |
| 13450 | I.S. | 0.41459 | 13451 | I.S. | 0.41457 | 13452 | I.S. | 0.41457 |
| 13453 | I.S. | 0.41455 | 13454 | I.S. | 0.41453 | 13455 | I.S. | 0.41452 |
| 13456 | I.S. | 0.41451 | 13457 | I.S. | 0.4145 | 13458 | ADA | 0.41447 |
| 13459 | I.S. | 0.41446 | 13460 | I.S. | 0.41444 | 13461 | I.S. | 0.41443 |
| 13462 | I.S. | 0.41441 | 13463 | I.S. | 0.41437 | 13464 | FANCM | 0.41435 |
| 13465 | I.S. | 0.41435 | 13466 | I.S. | 0.41435 | 13467 | I.S. | 0.41431 |
| 13468 | I.S. | 0.41431 | 13469 | I.S. | 0.41431 | 13470 | I.S. | 0.41423 |
| 13471 | KEL | 0.41423 | 13472 | RB1 | 0.41417 | 13473 | I.S. | 0.41416 |
| 13474 | I.S. | 0.41415 | 13475 | I.S. | 0.41411 | 13476 | I.S. | 0.41408 |
| 13477 | I.S. | 0.41408 | 13478 | I.S. | 0.41408 | 13479 | I.S. | 0.41407 |
| 13480 | I.S. | 0.41407 | 13481 | I.S. | 0.41405 | 13482 | I.S. | 0.41401 |
| 13483 | I.S. | 0.41399 | 13484 | I.S. | 0.41398 | 13485 | ANKRD26 | 0.41397 |
| 13486 | I.S. | 0.41396 | 13487 | LYST | 0.41396 | 13488 | I.S. | 0.41395 |
| 13489 | I.S. | 0.41393 | 13490 | FANCA | 0.41393 | 13491 | — | 0.4139 |
| 13492 | I.S. | 0.4139 | 13493 | I.S. | 0.41389 | 13494 | I.S. | 0.41388 |
| 13495 | I.S. | 0.41388 | 13496 | I.S. | 0.41387 | 13497 | SYK | 0.41387 |
| 13498 | I.S. | 0.41385 | 13499 | I.S. | 0.41385 | 13500 | I.S. | 0.41384 |
| 13501 | CTNNB1 | 0.41382 | 13502 | I.S. | 0.41381 | 13503 | ANKRD26 | 0.4138 |
| 13504 | I.S. | 0.4138 | 13505 | I.S. | 0.41379 | 13506 | I.S. | 0.41374 |
| 13507 | STAT4 | 0.41369 | 13508 | I.S. | 0.41369 | 13509 | I.S. | 0.41367 |
| 13510 | I.S. | 0.41366 | 13511 | I.S. | 0.41366 | 13512 | I.S. | 0.41363 |
| 13513 | SEC23B | 0.41361 | 13514 | I.S. | 0.41361 | 13515 | I.S. | 0.4136 |
| 13516 | I.S. | 0.41358 | 13517 | CUX1 | 0.41358 | 13518 | I.S. | 0.41354 |
| 13519 | EPHA7 | 0.41354 | 13520 | RET | 0.41353 | 13521 | KDM6A | 0.41352 |
| 13522 | FANCL | 0.41349 | 13523 | I.S. | 0.41348 | 13524 | I.S. | 0.41344 |
| 13525 | ATM | 0.41342 | 13526 | I.S. | 0.41341 | 13527 | I.S. | 0.4134 |
| 13528 | I.S. | 0.41339 | 13529 | I.S. | 0.41338 | 13530 | I.S. | 0.41334 |
| 13531 | I.S. | 0.41333 | 13532 | BCYRN1, | 0.41331 | 13533 | ATM | 0.41331 |
| | | | | TAF1 | | | | |
| 13534 | I.S. | 0.41328 | 13535 | I.S. | 0.41327 | 13536 | I.S. | 0.41325 |
| 13537 | BCYRN1, | 0.41325 | 13538 | I.S. | 0.41323 | 13539 | I.S. | 0.41322 |
| | TAF1 | | | | | | | |
| 13540 | I.S. | 0.41316 | 13541 | I.S. | 0.41316 | 13542 | MED12 | 0.41314 |
| 13543 | I.S. | 0.41313 | 13544 | I.S. | 0.41312 | 13545 | FGFR2 | 0.41311 |
| 13546 | RBBP6 | 0.4131 | 13547 | I.S. | 0.4131 | 13548 | I.S. | 0.41307 |
| 13549 | SMC3 | 0.41306 | 13550 | I.S. | 0.41306 | 13551 | I.S. | 0.41304 |
| 13552 | I.S. | 0.41301 | 13553 | I.S. | 0.41298 | 13554 | ATRX | 0.41298 |
| 13555 | I.S. | 0.41296 | 13556 | I.S. | 0.41295 | 13557 | I.S. | 0.41295 |
| 13558 | I.S. | 0.41292 | 13559 | I.S. | 0.41292 | 13560 | I.S. | 0.41292 |
| 13561 | I.S. | 0.41287 | 13562 | I.S. | 0.41287 | 13563 | I.S. | 0.41281 |
| 13564 | I.S. | 0.41279 | 13565 | I.S. | 0.41278 | 13566 | I.S. | 0.41278 |
| 13567 | SUZ12 | 0.41274 | 13568 | CIC | 0.41274 | 13569 | BAP1 | 0.41271 |
| 13570 | I.S. | 0.41271 | 13571 | TERF1 | 0.41269 | 13572 | I.S. | 0.41266 |
| 13573 | HSD3B1 | 0.41265 | 13574 | I.S. | 0.41265 | 13575 | I.S. | 0.41262 |
| 13576 | I.S. | 0.41262 | 13577 | I.S. | 0.41262 | 13578 | SMO | 0.41262 |
| 13579 | I.S. | 0.41261 | 13580 | I.S. | 0.4126 | 13581 | FAS | 0.41259 |
| 13582 | I.S. | 0.41258 | 13583 | GABRA6 | 0.41257 | 13584 | I.S. | 0.41255 |
| 13585 | I.S. | 0.41251 | 13586 | I.S. | 0.4125 | 13587 | I.S. | 0.41247 |
| 13588 | I.S. | 0.41246 | 13589 | I.S. | 0.4124 | 13590 | I.S. | 0.41236 |
| 13591 | JAK3 | 0.41236 | 13592 | I.S. | 0.41233 | 13593 | I.S. | 0.41233 |
| 13594 | I.S. | 0.41229 | 13595 | I.S. | 0.41229 | 13596 | GFI1B | 0.41227 |
| 13597 | I.S. | 0.41226 | 13598 | I.S. | 0.41224 | 13599 | SPTA1 | 0.41224 |
| 13600 | I.S. | 0.41218 | 13601 | I.S. | 0.41218 | 13602 | I.S. | 0.41217 |
| 13603 | I.S. | 0.41215 | 13604 | ARAF | 0.41215 | 13605 | I.S. | 0.41214 |
| 13606 | I.S. | 0.41212 | 13607 | SETD2 | 0.41212 | 13608 | RAB27A | 0.41209 |
| 13609 | LRP1B | 0.41208 | 13610 | I.S. | 0.41208 | 13611 | I.S. | 0.41205 |
| 13612 | I.S. | 0.41201 | 13613 | EP300 | 0.41201 | 13614 | I.S. | 0.41199 |
| 13615 | I.S. | 0.41197 | 13616 | PALB2 | 0.41197 | 13617 | I.S. | 0.41194 |
| 13618 | I.S. | 0.41191 | 13619 | GEN1 | 0.41188 | 13620 | I.S. | 0.41188 |
| 13621 | I.S. | 0.41188 | 13622 | I.S. | 0.41186 | 13623 | I.S. | 0.41183 |
| 13624 | I.S. | 0.41183 | 13625 | I.S. | 0.41182 | 13626 | I.S. | 0.41182 |
| 13627 | I.S. | 0.4118 | 13628 | I.S. | 0.4118 | 13629 | FANCC | 0.41179 |
| 13630 | I.S. | 0.41177 | 13631 | I.S. | 0.41177 | 13632 | I.S. | 0.41176 |
| 13633 | I.S. | 0.41174 | 13634 | RET | 0.4117 | 13635 | I.S. | 0.41168 |
| 13636 | I.S. | 0.41167 | 13637 | I.S. | 0.41167 | 13638 | I.S. | 0.41165 |
| 13639 | I.S. | 0.41164 | 13640 | KMT2D | 0.41162 | 13641 | I.S. | 0.41158 |
| 13642 | I.S. | 0.41158 | 13643 | I.S. | 0.4115 | 13644 | I.S. | 0.4115 |
| 13645 | I.S. | 0.41149 | 13646 | RAD54L | 0.41144 | 13647 | I.S. | 0.41144 |
| 13648 | I.S. | 0.4114 | 13649 | I.S. | 0.41138 | 13650 | PRKDC | 0.41135 |
| 13651 | I.S. | 0.41135 | 13652 | I.S. | 0.41133 | 13653 | I.S. | 0.41132 |
| 13654 | I.S. | 0.41131 | 13655 | ATRX | 0.4113 | 13656 | I.S. | 0.41129 |
| 13657 | MSH6 | 0.41128 | 13658 | I.S. | 0.41128 | 13659 | I.S. | 0.41128 |
| 13660 | LRP1B | 0.41126 | 13661 | I.S. | 0.41126 | 13662 | SPEN | 0.41122 |
| 13663 | I.S. | 0.41121 | 13664 | I.S. | 0.41121 | 13665 | PTEN | 0.4112 |
| 13666 | MTOR | 0.4112 | 13667 | I.S. | 0.41117 | 13668 | I.S. | 0.41108 |
| 13669 | I.S. | 0.41106 | 13670 | I.S. | 0.41102 | 13671 | I.S. | 0.41101 |
| 13672 | I.S. | 0.41099 | 13673 | I.S. | 0.41096 | 13674 | I.S. | 0.41095 |
| 13675 | XPO1 | 0.41093 | 13676 | SDHC | 0.41093 | 13677 | I.S. | 0.41092 |
| 13678 | I.S. | 0.41084 | 13679 | FANCD2 | 0.41084 | 13680 | I.S. | 0.41084 |
| 13681 | I.S. | 0.41084 | 13682 | I.S. | 0.41081 | 13683 | I.S. | 0.41081 |
| 13684 | I.S. | 0.41081 | 13685 | I.S. | 0.41081 | 13686 | PIK3CG | 0.41079 |
| 13687 | I.S. | 0.41076 | 13688 | I.S. | 0.41076 | 13689 | I.S. | 0.41075 |
| 13690 | BRD4 | 0.41075 | 13691 | CUX1 | 0.41074 | 13692 | I.S. | 0.41071 |
| 13693 | I.S. | 0.41069 | 13694 | I.S. | 0.41069 | 13695 | I.S. | 0.41069 |
| 13696 | I.S. | 0.41066 | 136971 | I.S. | 0.41065 | 13698 | I.S. | 0.41055 |
| 13699 | I.S. | 0.41054 | 13700 | I.S. | 0.41051 | 13701 | I.S. | 0.41051 |
| 13702 | ATR | 0.41048 | 13703 | KMT2C | 0.41048 | 13704 | I.S. | 0.41047 |
| 13705 | I.S. | 0.41045 | 13706 | I.S. | 0.41043 | 13707 | RANBP2 | 0.4104 |
| 13708 | I.S. | 0.4104 | 13709 | I.S. | 0.4104 | 13710 | TERF1 | 0.41037 |
| 13711 | I.S. | 0.41036 | 13712 | I.S. | 0.41036 | 13713 | I.S. | 0.41035 |
| 13714 | I.S. | 0.41033 | 13715 | I.S. | 0.41033 | 13716 | I.S. | 0.41031 |
| 13717 | I.S. | 0.4103 | 13718 | I.S. | 0.41027 | 13719 | ALK | 0.41027 |
| 13720 | I.S. | 0.41023 | 13721 | I.S. | 0.41021 | 13722 | CTCF | 0.41021 |
| 13723 | NOTCH3 | 0.41019 | 13724 | I.S. | 0.41018 | 13725 | CHEK2 | 0.41017 |
| 13726 | MSH2 | 0.41016 | 13727 | I.S. | 0.41014 | 13728 | I.S. | 0.41011 |
| 13729 | I.S. | 0.4101 | 13730 | G6PC3 | 0.4101 | 13731 | I.S. | 0.4101 |
| 13732 | I.S. | 0.41007 | 13733 | MSH6 | 0.41004 | 13734 | I.S. | 0.41004 |
| 13735 | I.S. | 0.41001 | 13736 | I.S. | 0.40998 | 13737 | NOTCH1 | 0.40996 |
| 13738 | I.S. | 0.40995 | 13739 | I.S. | 0.40992 | 13740 | BTK | 0.40992 |
| 13741 | I.S. | 0.40991 | 13742 | I.S. | 0.40991 | 13743 | I.S. | 0.40989 |
| 13744 | I.S. | 0.40988 | 13745 | I.S. | 0.40988 | 13746 | PRKDC | 0.40988 |
| 13747 | LRP1B | 0.40986 | 13748 | I.S. | 0.40986 | 13749 | I.S. | 0.40983 |
| 13750 | I.S. | 0.40983 | 13751 | I.S. | 0.4098 | 13752 | ERRFI1 | 0.40978 |
| 13753 | I.S. | 0.40976 | 13754 | I.S. | 0.40974 | 13755 | I.S. | 0.40973 |
| 13756 | I.S. | 0.4097 | 13757 | I.S. | 0.4097 | 13758 | I.S. | 0.40966 |
| 13759 | I.S. | 0.40965 | 13760 | I.S. | 0.40965 | 13761 | I.S. | 0.40963 |
| 13762 | I.S. | 0.40962 | 13763 | I.S. | 0.40962 | 13764 | KDM5C, | 0.40959 |
| | | | | | | | MIR6895 | |
| 13765 | I.S. | 0.40959 | 13766 | I.S. | 0.40959 | 13767 | I.S. | 0.40954 |
| 13768 | I.S. | 0.40953 | 13769 | TOP1 | 0.40953 | 13770 | I.S. | 0.40953 |
| 13771 | I.S. | 0.40953 | 13772 | I.S. | 0.4095 | 13773 | I.S. | 0.4095 |
| 13774 | PRKDC | 0.40948 | 13775 | KMT2D | 0.40948 | 13776 | I.S. | 0.40948 |
| 13777 | I.S. | 0.40947 | 13778 | I.S. | 0.40947 | 13779 | I.S. | 0.40945 |
| 13780 | I.S. | 0.40945 | 13781 | I.S. | 0.40942 | 13782 | I.S. | 0.40942 |
| 13783 | RANBP2 | 0.40941 | 13784 | I.S. | 0.40941 | 13785 | I.S. | 0.40941 |
| 13786 | I.S. | 0.4094 | 13787 | I.S. | 0.4094 | 13788 | I.S. | 0.40939 |
| 13789 | BCOR | 0.40939 | 13790 | I.S. | 0.40938 | 13791 | I.S. | 0.40938 |
| 13792 | I.S. | 0.40937 | 13793 | I.S. | 0.40936 | 13794 | I.S. | 0.40936 |
| 13795 | KDM6A | 0.40936 | 13796 | I.S. | 0.40936 | 13797 | I.S. | 0.40935 |
| 13798 | I.S. | 0.40934 | 13799 | I.S. | 0.4093 | 13800 | I.S. | 0.4093 |
| 13801 | I.S. | 0.40924 | 13802 | I.S. | 0.40923 | 13803 | I.S. | 0.40921 |
| 13804 | I.S. | 0.40917 | 13805 | I.S. | 0.40915 | 13806 | RANBP2 | 0.40914 |
| 13807 | I.S. | 0.40914 | 13808 | I.S. | 0.40912 | 13809 | MED12 | 0.40912 |
| 13810 | I.S. | 0.40909 | 13811 | I.S. | 0.40909 | 13812 | I.S. | 0.40904 |
| 13813 | I.S. | 0.409 | 13814 | I.S. | 0.409 | 13815 | I.S. | 0.40895 |
| 13816 | I.S. | 0.40895 | 13817 | WT1 | 0.40893 | 13818 | I.S. | 0.40892 |
| 13819 | I.S. | 0.40891 | 13820 | I.S. | 0.40888 | 13821 | I.S. | 0.40884 |
| 13822 | AXL | 0.40882 | 13823 | I.S. | 0.40879 | 13824 | I.S. | 0.40879 |
| 13825 | PALB2 | 0.40873 | 13826 | I.S. | 0.4087 | 13827 | I.S. | 0.4087 |
| 13828 | I.S. | 0.4087 | 13829 | I.S. | 0.40867 | 13830 | RPTOR | 0.40864 |
| 13831 | I.S. | 0.40863 | 13832 | SEC23B | 0.40863 | 13833 | I.S. | 0.40861 |
| 13834 | I.S. | 0.40861 | 13835 | TNFAIP3 | 0.40858 | 13836 | I.S. | 0.40856 |
| 13837 | PTCH1 | 0.40856 | 13838 | LRP1B | 0.40852 | 13839 | I.S. | 0.4085 |
| 13840 | I.S. | 0.4085 | 13841 | GATA3 | 0.40849 | 13842 | I.S. | 0.40849 |
| 13843 | I.S. | 0.40847 | 13844 | I.S. | 0.40847 | 13845 | RBM10 | 0.40844 |
| 13846 | I.S. | 0.40843 | 13847 | I.S. | 0.40843 | 13848 | SMC1A | 0.40841 |
| 13849 | I.S. | 0.40841 | 13850 | SEC23B | 0.4084 | 13851 | I.S. | 0.4084 |
| 13852 | I.S. | 0.40837 | 13853 | I.S. | 0.40836 | 13854 | I.S. | 0.40835 |
| 13855 | BAP1 | 0.40834 | 13856 | I.S. | 0.40831 | 13857 | I.S. | 0.40826 |
| 13858 | I.S. | 0.40825 | 13859 | I.S. | 0.40825 | 13860 | I.S. | 0.40823 |
| 13861 | I.S. | 0.40818 | 13862 | NTRK1 | 0.40815 | 13863 | I.S. | 0.40814 |
| 13864 | I.S. | 0.40814 | 13865 | RAD50 | 0.40814 | 13866 | I.S. | 0.40813 |
| 13867 | I.S. | 0.40811 | 13868 | I.S. | 0.4081 | 13869 | I.S. | 0.4081 |
| 13870 | ZNF703 | 0.40808 | 13871 | I.S. | 0.40805 | 13872 | I.S. | 0.40805 |
| 13873 | I.S. | 0.40805 | 13874 | I.S. | 0.40802 | 13875 | I.S. | 0.40802 |
| 13876 | PRKDC | 0.40802 | 13877 | I.S. | 0.408 | 13878 | ZRSR2 | 0.408 |
| 13879 | I.S. | 0.40799 | 13880 | I.S. | 0.40789 | 13881 | KMT2D | 0.40787 |
| 13882 | PTEN | 0.40785 | 13883 | XPO1 | 0.40784 | 13884 | I.S. | 0.40781 |
| 13885 | I.S. | 0.40781 | 13886 | I.S. | 0.4078 | 13887 | FANCC | 0.40778 |
| 13888 | I.S. | 0.40775 | 13889 | I.S. | 0.40773 | 13890 | I.S. | 0.40769 |
| 13891 | I.S. | 0.40767 | 13892 | I.S. | 0.40766 | 13893 | JAPC | 0.40765 |
| 13894 | I.S. | 0.40764 | 13895 | I.S. | 0.40764 | 13896 | PIK3R1 | 0.40754 |
| 13897 | FANCA | 0.40753 | 13898 | I.S. | 0.40752 | 13899 | EP300 | 0.40748 |
| 13900 | I.S. | 0.40748 | 13901 | I.S. | 0.40748 | 13902 | I.S. | 0.40748 |
| 13903 | I.S. | 0.40742 | 13904 | I.S. | 0.40739 | 13905 | I.S. | 0.40737 |
| 13906 | ARAF | 0.40734 | 13907 | I.S. | 0.40733 | 13908 | I.S. | 0.40731 |
| 13909 | I.S. | 0.40731 | 13910 | I.S. | 0.40728 | 13911 | I.S. | 0.40722 |
| 13912 | I.S. | 0.40722 | 13913 | I.S. | 0.40719 | 13914 | I.S. | 0.40719 |
| 13915 | I.S. | 0.40718 | 13916 | I.S. | 0.40718 | 13917 | I.S. | 0.40718 |
| 13918 | I.S. | 0.40717 | 13919 | I.S. | 0.40713 | 13920 | TOP1 | 0.40712 |
| 13921 | I.S. | 0.40711 | 13922 | I.S. | 0.4071 | 13923 | MAGI2 | 0.4071 |
| 13924 | I.S. | 0.4071 | 13925 | I.S. | 0.40709 | 13926 | GRIN2A | 0.40707 |
| 13927 | I.S. | 0.40705 | 13928 | I.S. | 0.40704 | 13929 | I.S. | 0.40703 |
| 13930 | I.S. | 0.40701 | 13931 | I.S. | 0.40701 | 13932 | I.S. | 0.407 |
| 13933 | I.S. | 0.407 | 13934 | I.S. | 0.407 | 13935 | MET | 0.40698 |
| 13936 | I.S. | 0.40695 | 13937 | I.S. | 0.40695 | 13938 | I.S. | 0.40694 |
| 13939 | I.S. | 0.40691 | 13940 | STAG2 | 0.40689 | 13941 | I.S. | 0.40688 |
| 13942 | I.S. | 0.40686 | 13943 | I.S. | 0.40684 | 13944 | EPHA5 | 0.40681 |
| 13945 | I.S. | 0.4068 | 13946 | PIK3CB | 0.40677 | 13947 | I.S. | 0.40677 |
| 13948 | I.S. | 0.40677 | 13949 | I.S. | 0.40676 | 13950 | I.S. | 0.40675 |
| 13951 | I.S. | 0.40674 | 13952 | MAP2K1, | 0.40671 | 13953 | BCYRN1, | 0.40668 |
| | | | | SNAPC5 | | | TAF1 | |
| 13954 | I.S. | 0.40665 | 13955 | I.S. | 0.40665 | 13956 | I.S. | 0.40665 |
| 13957 | I.S. | 0.40663 | 13958 | SF3B1 | 0.40662 | 13959 | I.S. | 0.40662 |
| 13960 | I.S. | 0.4066 | 13961 | I.S. | 0.40659 | 13962 | RHEB | 0.40657 |
| 13963 | I.S. | 0.40656 | 13964 | I.S. | 0.40656 | 13965 | I.S. | 0.40656 |
| 13966 | SETD2 | 0.40654 | 13967 | I.S. | 0.40654 | 13968 | KDR | 0.40654 |
| 13969 | I.S. | 0.40654 | 13970 | I.S. | 0.40653 | 13971 | I.S. | 0.40649 |
| 13972 | KMT2B | 0.40647 | 13973 | NF1 | 0.40646 | 13974 | I.S. | 0.40645 |
| 13975 | I.S. | 0.40644 | 13976 | EGFR | 0.40642 | 13977 | KIF23 | 0.40641 |
| 13978 | I.S. | 0.40641 | 13979 | I.S. | 0.40638 | 13980 | I.S. | 0.40638 |
| 13981 | I.S. | 0.40636 | 13982 | GALNT12 | 0.40636 | 13983 | I.S. | 0.40635 |
| 13984 | PREX2 | 0.40633 | 13985 | I.S. | 0.40633 | 13986 | FANCM | 0.4063 |
| 13987 | I.S. | 0.40627 | 13988 | BCYRN1, | 0.40627 | 13989 | I.S. | 0.40627 |
| | | | | TAF1 | | | | |
| 13990 | DNMT3A | 0.40626 | 13991 | I.S. | 0.40621 | 13992 | PMS1 | 0.40619 |
| 13993 | I.S. | 0.40618 | 13994 | I.S. | 0.40617 | 13995 | I.S. | 0.40616 |
| 13996 | I.S. | 0.40616 | 13997 | I.S. | 0.40614 | 13998 | I.S. | 0.40612 |
| 13999 | I.S. | 0.40612 | 14000 | I.S. | 0.40611 | 14001 | I.S. | 0.40608 |
| 14002 | I.S. | 0.40605 | 14003 | I.S. | 0.40605 | 14004 | DDX11 | 0.40603 |
| 14005 | I.S. | 0.40602 | 14006 | I.S. | 0.406 | 14007 | I.S. | 0.406 |
| 14008 | I.S. | 0.40599 | 14009 | I.S. | 0.40598 | 14010 | I.S. | 0.40597 |
| 14011 | I.S. | 0.40597 | 14012 | I.S. | 0.40594 | 14013 | FANCG | 0.40591 |
| 14014 | I.S. | 0.40588 | 14015 | I.S. | 0.40579 | 14016 | I.S. | 0.40576 |
| 14017 | I.S. | 0.40573 | 14018 | I.S. | 0.40572 | 14019 | I.S. | 0.40572 |
| 14020 | I.S. | 0.4057 | 14021 | I.S. | 0.4057 | 14022 | I.S. | 0.4057 |
| 14023 | I.S. | 0.40565 | 14024 | I.S. | 0.40565 | 14025 | I.S. | 0.40564 |
| 14026 | I.S. | 0.40564 | 14027 | I.S. | 0.40562 | 14028 | I.S. | 0.40561 |
| 14029 | I.S. | 0.40558 | 14030 | POLE | 0.40558 | 14031 | I.S. | 0.40558 |
| 14032 | I.S. | 0.40557 | 14033 | GID4 | 0.40556 | 14034 | I.S. | 0.40555 |
| 14035 | CHD4 | 0.40552 | 14036 | I.S. | 0.40551 | 14037 | MTOR | 0.4055 |
| 14038 | I.S. | 0.4055 | 14039 | I.S. | 0.40549 | 14040 | I.S. | 0.40543 |
| 14041 | DOT1L | 0.40542 | 14042 | I.S. | 0.4054 | 14043 | I.S. | 0.4054 |
| 14044 | I.S. | 0.40537 | 14045 | I.S. | 0.40537 | 14046 | CYLD | 0.40535 |
| 14047 | I.S. | 0.40534 | 14048 | I.S. | 0.40531 | 14049 | I.S. | 0.40531 |
| 14050 | I.S. | 0.4053 | 14051 | I.S. | 0.40529 | 14052 | I.S. | 0.40529 |
| 14053 | I.S. | 0.40529 | 14054 | I.S. | 0.40529 | 14055 | KIT | 0.40526 |
| 14056 | I.S. | 0.40525 | 14057 | I.S. | 0.40522 | 14058 | I.S. | 0.40521 |
| 14059 | I.S. | 0.4052 | 14060 | — | 0.40519 | 14061 | I.S. | 0.40519 |
| 14062 | CD79A | 0.40516 | 14063 | I.S. | 0.40514 | 14064 | I.S. | 0.40509 |
| 14065 | I.S. | 0.40508 | 14066 | CHEK2 | 0.40508 | 14067 | I.S. | 0.40508 |
| 14068 | I.S. | 0.40507 | 14069 | I.S. | 0.40506 | 14070 | I.S. | 0.40497 |
| 14071 | I.S. | 0.40493 | 14072 | I.S. | 0.40493 | 14073 | I.S. | 0.40492 |
| 14074 | STAG2 | 0.40491 | 14075 | FUBP1 | 0.4049 | 14076 | STAG2 | 0.4049 |
| 14077 | I.S. | 0.40489 | 14078 | JAK1 | 0.40488 | 14079 | I.S. | 0.40485 |
| 14080 | I.S. | 0.40484 | 14081 | SPEN | 0.40481 | 14082 | SYK | 0.40481 |
| 14083 | I.S. | 0.40481 | 14084 | I.S. | 0.40477 | 14085 | I.S. | 0.40476 |
| 14086 | I.S. | 0.40475 | 14087 | I.S. | 0.40475 | 14088 | I.S. | 0.40473 |
| 14089 | I.S. | 0.40468 | 14090 | I.S. | 0.40468 | 14091 | I.S. | 0.40466 |
| 14092 | LRP1B | 0.40464 | 14093 | I.S. | 0.40463 | 14094 | I.S. | 0.40463 |
| 14095 | I.S. | 0.4046 | 14096 | I.S. | 0.4046 | 14097 | I.S. | 0.4046 |
| 14098 | I.S. | 0.40458 | 14099 | CHD2 | 0.40456 | 14100 | I.S. | 0.40455 |
| 14101 | I.S. | 0.40454 | 14102 | ARID1A | 0.40449 | 14103 | I.S. | 0.40449 |
| 14104 | EZH2 | 0.40443 | 14105 | I.S. | 0.40442 | 14106 | I.S. | 0.40442 |
| 14107 | I.S. | 0.40442 | 14108 | I.S. | 0.40441 | 14109 | NPM1 | 0.40436 |
| 14110 | I.S. | 0.40436 | 14111 | I.S. | 0.40435 | 14112 | I.S. | 0.4043 |
| 14113 | BCYRN1, | 0.40429 | 14114 | I.S. | 0.40426 | 14115 | I.S. | 0.40425 |
| | TAF1 | | | | | | | |
| 14116 | MEFV | 0.40424 | 14117 | I.S. | 0.40422 | 14118 | TGFBR2 | 0.40422 |
| 14119 | I.S. | 0.40422 | 14120 | EPHA5 | 0.40415 | 14121 | CDK6 | 0.40413 |
| 14122 | I.S. | 0.40412 | 14123 | I.S. | 0.4041 | 14124 | ABL1 | 0.4041 |
| 14125 | I.S. | 0.40407 | 14126 | I.S. | 0.40406 | 14127 | I.S. | 0.40401 |
| 14128 | I.S. | 0.40401 | 14129 | I.S. | 0.40401 | 14130 | I.S. | 0.404 |
| 14131 | I.S. | 0.40398 | 14132 | I.S. | 0.40398 | 14133 | CUX1 | 0.40398 |
| 14134 | I.S. | 0.40397 | 14135 | I.S. | 0.40397 | 14136 | I.S. | 0.40397 |
| 14137 | I.S. | 0.40397 | 14138 | I.S. | 0.40396 | 14139 | I.S. | 0.40396 |
| 14140 | FANCB | 0.40392 | 14141 | ZRSR2 | 0.40392 | 14142 | PHF6 | 0.40389 |
| 14143 | I.S. | 0.40386 | 14144 | I.S. | 0.40386 | 14145 | I.S. | 0.40385 |
| 14146 | I.S. | 0.40382 | 14147 | I.S. | 0.40381 | 14148 | ROS1 | 0.40381 |
| 14149 | I.S. | 0.4038 | 14150 | I.S. | 0.40377 | 14151 | I.S. | 0.40377 |
| 14152 | SMC3 | 0.40375 | 14153 | I.S. | 0.40369 | 14154 | I.S. | 0.40366 |
| 14155 | PRKDC | 0.40365 | 14156 | NOTCH3 | 0.40362 | 14157 | I.S. | 0.4036 |
| 14158 | I.S. | 0.4036 | 14159 | I.S. | 0.40357 | 14160 | I.S. | 0.40356 |
| 14161 | I.S. | 0.40354 | 14162 | I.S. | 0.40353 | 14163 | RICTOR | 0.40353 |
| 14164 | I.S. | 0.40351 | 14165 | I.S. | 0.4035 | 14166 | I.S. | 0.4035 |
| 14167 | I.S. | 0.40347 | 14168 | RANBP2 | 0.40342 | 14169 | STAG2 | 0.40342 |
| 14170 | PRKDC | 0.40342 | 14171 | I.S. | 0.4034 | 14172 | I.S. | 0.4034 |
| 14173 | I.S. | 0.40336 | 14174 | EPHA3 | 0.40335 | 14175 | I.S. | 0.40332 |
| 14176 | I.S. | 0.40332 | 14177 | I.S. | 0.4033 | 14178 | I.S. | 0.40329 |
| 14179 | I.S. | 0.40327 | 14180 | MTOR | 0.40325 | 14181 | LRP1B | 0.40324 |
| 14182 | I.S. | 0.40324 | 14183 | I.S. | 0.40321 | 14184 | I.S. | 0.4032 |
| 14185 | I.S. | 0.40319 | 14186 | MED12 | 0.40318 | 14187 | I.S. | 0.40315 |
| 14188 | I.S. | 0.40312 | 14189 | I.S. | 0.40312 | 14190 | I.S. | 0.40312 |
| 14191 | I.S. | 0.40308 | 14192 | I.S. | 0.40307 | 14193 | I.S. | 0.40306 |
| 14194 | I.S. | 0.40306 | 14195 | I.S. | 0.40304 | 14196 | I.S. | 0.40304 |
| 14197 | I.S. | 0.40304 | 14198 | I.S. | 0.40303 | 14199 | I.S. | 0.40302 |
| 14200 | I.S. | 0.40299 | 14201 | I.S. | 0.40297 | 14202 | AR | 0.40297 |
| 14203 | I.S. | 0.40297 | 14204 | I.S. | 0.40294 | 14205 | I.S. | 0.40294 |
| 14206 | I.S. | 0.40294 | 14207 | I.S. | 0.40294 | 14208 | I.S. | 0.40293 |
| 14209 | I.S. | 0.40291 | 14210 | RTEL1 | 0.40291 | 14211 | I.S. | 0.40289 |
| 14212 | I.S. | 0.40287 | 14213 | I.S. | 0.40285 | 14214 | I.S. | 0.40283 |
| 14215 | I.S. | 0.40279 | 14216 | KEL | 0.40273 | 14217 | I.S. | 0.40268 |
| 14218 | NRAS | 0.40268 | 14219 | I.S. | 0.40266 | 14220 | I.S. | 0.40266 |
| 14221 | ATRX | 0.40261 | 14222 | I.S. | 0.40259 | 14223 | I.S. | 0.40258 |
| 14224 | I.S. | 0.40258 | 14225 | I.S. | 0.40258 | 14226 | I.S. | 0.40257 |
| 14227 | I.S. | 0.40255 | 14228 | I.S. | 0.40255 | 14229 | I.S. | 0.40255 |
| 14230 | I.S. | 0.40254 | 14231 | KDM6A | 0.40253 | 14232 | I.S. | 0.40252 |
| 14233 | I.S. | 0.40249 | 14234 | I.S. | 0.40249 | 14235 | I.S. | 0.40249 |
| 14236 | CBLB | 0.40247 | 14237 | I.S. | 0.40247 | 14238 | SETD2 | 0.40246 |
| 14239 | I.S. | 0.40246 | 14240 | I.S. | 0.40243 | 14241 | I.S. | 0.40241 |
| 14242 | STAG2 | 0.40241 | 14243 | I.S. | 0.40236 | 14244 | BTG1 | 0.40236 |
| 14245 | I.S. | 0.40232 | 14246 | I.S. | 0.40232 | 14247 | TSC2 | 0.40228 |
| 14248 | BRAF | 0.40228 | 14249 | GRIN2A | 0.40223 | 14250 | ARID1A | 0.40223 |
| 14251 | I.S. | 0.40222 | 14252 | I.S. | 0.40222 | 14253 | I.S. | 0.40221 |
| 14254 | I.S. | 0.4022 | 14255 | I.S. | 0.4022 | 14256 | NOTCH1 | 0.40217 |
| 14257 | I.S. | 0.40217 | 14258 | I.S. | 0.40217 | 14259 | I.S. | 0.40215 |
| 14260 | RICTOR | 0.40214 | 14261 | DNMT3A | 0.40213 | 14262 | KDR | 0.40211 |
| 14263 | INPP4B | 0.40211 | 14264 | I.S. | 0.4021 | 14265 | I.S. | 0.4021 |
| 14266 | I.S. | 0.4021 | 14267 | INPP4B | 0.40209 | 14268 | I.S. | 0.40208 |
| 14269 | I.S. | 0.40205 | 14270 | I.S. | 0.40205 | 14271 | I.S. | 0.40205 |
| 14272 | I.S. | 0.40205 | 14273 | I.S. | 0.40203 | 14274 | FANCC | 0.40196 |
| 14275 | I.S. | 0.40196 | 14276 | CREBBP | 0.40195 | 14277 | I.S. | 0.40187 |
| 14278 | I.S. | 0.40187 | 14279 | I.S. | 0.40184 | 14280 | I.S. | 0.40184 |
| 14281 | I.S. | 0.40184 | 14282 | I.S. | 0.40183 | 14283 | I.S. | 0.40182 |
| 14284 | I.S. | 0.40181 | 14285 | I.S. | 0.40179 | 14286 | I.S. | 0.40179 |
| 14287 | WAS | 0.40178 | 14288 | I.S. | 0.40178 | 14289 | I.S. | 0.40175 |
| 14290 | I.S. | 0.40175 | 14291 | I.S. | 0.40175 | 14292 | SMAD3 | 0.40175 |
| 14293 | I.S. | 0.40175 | 14294 | I.S. | 0.40174 | 14295 | I.S. | 0.40172 |
| 14296 | I.S. | 0.40169 | 14297 | I.S. | 0.40169 | 14298 | I.S. | 0.40167 |
| 14299 | I.S. | 0.40163 | 14300 | I.S. | 0.40162 | 14301 | I.S. | 0.4016 |
| 14302 | I.S. | 0.4016 | 14303 | I.S. | 0.40158 | 14304 | I.S. | 0.40154 |
| 14305 | I.S. | 0.40154 | 14306 | I.S. | 0.40151 | 14307 | I.S. | 0.40151 |
| 14308 | I.S. | 0.40151 | 14309 | I.S. | 0.40145 | 14310 | I.S. | 0.40145 |
| 14311 | NTRK2 | 0.40143 | 14312 | I.S. | 0.40143 | 14313 | RBBP6 | 0.40143 |
| 14314 | I.S. | 0.40139 | 14315 | I.S. | 0.40138 | 14316 | I.S. | 0.40136 |
| 14317 | I.S. | 0.40134 | 14318 | I.S. | 0.40134 | 14319 | I.S. | 0.40134 |
| 14320 | I.S. | 0.40133 | 14321 | CBLB | 0.40133 | 14322 | I.S. | 0.40129 |
| 14323 | I.S. | 0.40129 | 14324 | I.S. | 0.40128 | 14325 | I.S. | 0.40127 |
| 14326 | I.S. | 0.40127 | 14327 | I.S. | 0.40127 | 14328 | I.S. | 0.40124 |
| 14329 | PMS2 | 0.40122 | 14330 | NF2 | 0.40122 | 14331 | I.S. | 0.40118 |
| 14332 | I.S. | 0.40118 | 14333 | I.S. | 0.40118 | 14334 | I.S. | 0.40118 |
| 14335 | I.S. | 0.40115 | 14336 | I.S. | 0.40115 | 14337 | I.S. | 0.40112 |
| 14338 | PRKDC | 0.4011 | 14339 | I.S. | 0.4011 | 14340 | I.S. | 0.4011 |
| 14341 | I.S. | 0.40109 | 14342 | I.S. | 0.40107 | 14343 | I.S. | 0.40106 |
| 14344 | I.S. | 0.40103 | 14345 | I.S. | 0.40103 | 14346 | I.S. | 0.40103 |
| 14347 | FLT3 | 0.40102 | 14348 | CDK8 | 0.40102 | 14349 | I.S. | 0.40098 |
| 14350 | I.S. | 0.40097 | 14351 | PPM1D | 0.40097 | 14352 | I.S. | 0.40095 |
| 14353 | TCIRG1 | 0.40095 | 14354 | I.S. | 0.40094 | 14355 | I.S. | 0.40093 |
| 14356 | STK11 | 0.40092 | 14357 | I.S. | 0.4009 | 14358 | I.S. | 0.4009 |
| 14359 | CSF1R | 0.40089 | 14360 | CUX1 | 0.40089 | 14361 | PRKDC | 0.40089 |
| 14362 | I.S. | 0.40089 | 14363 | RNF43 | 0.40089 | 14364 | I.S. | 0.40088 |
| 14365 | I.S. | 0.40085 | 14366 | BCOR | 0.40083 | 14367 | I.S. | 0.40083 |
| 14368 | I.S. | 0.40081 | 14369 | RBBP6 | 0.40081 | 14370 | AXIN2 | 0.40077 |
| 14371 | I.S. | 0.40074 | 14372 | I.S. | 0.40073 | 14373 | I.S. | 0.40071 |
| 14374 | I.S. | 0.4007 | 14375 | I.S. | 0.40068 | 14376 | I.S. | 0.40067 |
| 14377 | SDHB | 0.40066 | 14378 | RAD51C | 0.40066 | 14379 | I.S. | 0.40065 |
| 14380 | PRKDC | 0.40059 | 14381 | I.S. | 0.40056 | 14382 | I.S. | 0.40055 |
| 14383 | I.S. | 0.40054 | 14384 | I.S. | 0.40053 | 14385 | I.S. | 0.40053 |
| 14386 | I.S. | 0.40053 | 14387 | I.S. | 0.40052 | 14388 | FANCI | 0.40051 |
| 14389 | I.S. | 0.4005 | 14390 | I.S. | 0.4005 | 14391 | I.S. | 0.40048 |
| 14392 | I.S. | 0.40047 | 14393 | I.S. | 0.40047 | 14394 | I.S. | 0.40047 |
| 14395 | CREBBP | 0.40047 | 14396 | I.S. | 0.40046 | 14397 | I.S. | 0.40044 |
| 14398 | I.S. | 0.40044 | 14399 | I.S. | 0.40042 | 14400 | I.S. | 0.40039 |
| 14401 | I.S. | 0.40038 | 14402 | I.S. | 0.40036 | 14403 | I.S. | 0.40034 |
| 14404 | I.S. | 0.40033 | 14405 | I.S. | 0.40031 | 14406 | I.S. | 0.4003 |
| 14407 | I.S. | 0.40029 | 14408 | I.S. | 0.40029 | 14409 | I.S. | 0.40024 |
| 14410 | I.S. | 0.40023 | 14411 | I.S. | 0.40023 | 14412 | I.S. | 0.40021 |
| 14413 | I.S. | 0.4002 | 14414 | I.S. | 0.4002 | 14415 | I.S. | 0.40019 |
| 14416 | STAG2 | 0.40018 | 14417 | I.S. | 0.40018 | 14418 | I.S. | 0.40015 |
| 14419 | I.S. | 0.40012 | 14420 | CYLD | 0.40012 | 14421 | I.S. | 0.40011 |
| 14422 | I.S. | 0.40011 | 14423 | I.S. | 0.40011 | 14424 | PHF6 | 0.4001 |
| 14425 | I.S. | 0.40006 | 14426 | FLT4 | 0.40004 | 14427 | PREX2 | 0.40004 |
| 14428 | I.S. | 0.40003 | 14429 | I.S. | 0.40002 | 14430 | I.S. | 0.4 |
| 14431 | I.S. | 0.4 | 14432 | SMC1A | 0.39998 | 14433 | PALB2 | 0.39998 |
| 14434 | I.S. | 0.39997 | 14435 | I.S. | 0.39996 | 14436 | I.S. | 0.39996 |
| 14437 | EPCAM | 0.39991 | 14438 | PALB2 | 0.39987 | 14439 | I.S. | 0.39985 |
| 14440 | TCIRG1 | 0.39985 | 14441 | I.S. | 0.39983 | 14442 | I.S. | 0.39982 |
| 14443 | AMER1 | 0.39979 | 14444 | I.S. | 0.39979 | 14445 | I.S. | 0.39979 |
| 14446 | I.S. | 0.39978 | 14447 | I.S. | 0.39978 | 14448 | I.S. | 0.39977 |
| 14449 | I.S. | 0.39973 | 14450 | DNM2, | 0.39973 | 14451 | ZNF217 | 0.39971 |
| | | | | MIR6793 | | | | |
| 14452 | I.S. | 0.3997 | 14453 | MED12 | 0.39968 | 14454 | I.S. | 0.39967 |
| 14455 | I.S. | 0.39965 | 14456 | I.S. | 0.39964 | 14457 | I.S. | 0.39964 |
| 14458 | I.S. | 0.39964 | 14459 | I.S. | 0.39962 | 14460 | I.S. | 0.39962 |
| 14461 | I.S. | 0.39962 | 14462 | I.S. | 0.3996 | 14463 | I.S. | 0.39958 |
| 14464 | I.S. | 0.39958 | 14465 | I.S. | 0.39957 | 14466 | I.S. | 0.39957 |
| 14467 | I.S. | 0.39954 | 14468 | NRAS | 0.39952 | 14469 | I.S. | 0.39949 |
| 14470 | KAT6A | 0.39949 | 14471 | I.S. | 0.39947 | 14472 | I.S. | 0.39946 |
| 14473 | I.S. | 0.39946 | 14474 | I.S. | 0.39942 | 14475 | MET | 0.39941 |
| 14476 | I.S. | 0.39941 | 14477 | KMT2A | 0.39937 | 14478 | I.S. | 0.39937 |
| 14479 | PRKDC | 0.39937 | 14480 | RB1 | 0.39937 | 14481 | I.S. | 0.39937 |
| 14482 | I.S. | 0.39934 | 14483 | I.S. | 0.39934 | 14484 | AXIN1 | 0.39932 |
| 14485 | I.S. | 0.39929 | 14486 | I.S. | 0.39929 | 14487 | JAK3 | 0.39929 |
| 14488 | I.S. | 0.39926 | 14489 | I.S. | 0.39926 | 14490 | I.S. | 0.39925 |
| 14491 | I.S. | 0.39922 | 14492 | FOXP1 | 0.39922 | 14493 | I.S. | 0.39922 |
| 14494 | EP300 | 0.39921 | 14495 | I.S. | 0.3992 | 14496 | I.S. | 0.3992 |
| 14497 | I.S. | 0.39917 | 14498 | I.S. | 0.39916 | 14499 | I.S. | 0.39916 |
| 14500 | I.S. | 0.39914 | 14501 | RTEL1 | 0.39914 | 14502 | I.S. | 0.39913 |
| 14503 | I.S. | 0.39908 | 14504 | I.S. | 0.39908 | 14505 | I.S. | 0.39907 |
| 14506 | I.S. | 0.39905 | 14507 | BLM | 0.39905 | 14508 | I.S. | 0.39902 |
| 14509 | GFI1B | 0.39902 | 14510 | I.S. | 0.39902 | 14511 | I.S. | 0.39902 |
| 14512 | JAK3 | 0.39902 | 14513 | LRP1B | 0.39901 | 14514 | I.S. | 0.39901 |
| 14515 | I.S. | 0.39899 | 14516 | I.S. | 0.39899 | 14517 | MAP3K1 | 0.39896 |
| 14518 | PRKDC | 0.39896 | 14519 | I.S. | 0.39896 | 14520 | I.S. | 0.39896 |
| 14521 | I.S. | 0.39896 | 14522 | I.S. | 0.39893 | 14523 | I.S. | 0.39893 |
| 14524 | I.S. | 0.39887 | 14525 | I.S. | 0.39884 | 14526 | I.S. | 0.39883 |
| 14527 | I.S. | 0.39881 | 14528 | I.S. | 0.39878 | 14529 | I.S. | 0.39875 |
| 14530 | CUX1 | 0.39875 | 14531 | I.S. | 0.39871 | 14532 | I.S. | 0.3987 |
| 14533 | I.S. | 0.3987 | 14534 | I.S. | 0.3987 | 14535 | I.S. | 0.39869 |
| 14536 | I.S. | 0.39867 | 14537 | I.S. | 0.39867 | 14538 | SPTA1 | 0.39866 |
| 14539 | I.S. | 0.39866 | 14540 | KMT2C | 0.39864 | 14541 | I.S. | 0.39863 |
| 14542 | I.S. | 0.39858 | 14543 | STAG2 | 0.39858 | 14544 | I.S. | 0.39857 |
| 14545 | I.S. | 0.39856 | 14546 | I.S. | 0.39855 | 14547 | FANCM | 0.39855 |
| 14548 | I.S. | 0.39853 | 14549 | I.S. | 0.39851 | 14550 | SF3B1 | 0.39843 |
| 14551 | I.S. | 0.39842 | 14552 | MED12 | 0.39842 | 14553 | I.S. | 0.39837 |
| 14554 | I.S. | 0.39837 | 14555 | APC | 0.39834 | 14556 | I.S. | 0.3983 |
| 14557 | I.S. | 0.39828 | 14558 | I.S. | 0.39826 | 14559 | I.S. | 0.39825 |
| 14560 | BCYRN1, | 0.39825 | 14561 | I.S. | 0.39825 | 14562 | I.S. | 0.39824 |
| | TAF1 | | | | | | | |
| 14563 | BCYRN1, | 0.39824 | 14564 | DAXX | 0.39823 | 14565 | ATM | 0.39822 |
| | TAF1 | | | | | | | |
| 14566 | I.S. | 0.39822 | 14567 | I.S. | 0.39819 | 14568 | I.S. | 0.39816 |
| 14569 | I.S. | 0.39809 | 14570 | I.S. | 0.39809 | 14571 | I.S. | 0.39809 |
| 14572 | I.S. | 0.39808 | 14573 | TNFAIP3 | 0.39806 | 14574 | I.S. | 0.39804 |
| 14575 | U2AF1, | 0.39804 | 14576 | I.S. | 0.39804 | 14577 | I.S. | 0.39804 |
| | U2AF1L5 | | | | | | | |
| 14578 | I.S. | 0.39803 | 14579 | I.S. | 0.39801 | 14580 | I.S. | 0.39801 |
| 14581 | I.S. | 0.39801 | 14582 | I.S. | 0.39801 | 14583 | I.S. | 0.39797 |
| 14584 | I.S. | 0.39796 | 14585 | I.S. | 0.39795 | 14586 | I.S. | 0.39795 |
| 14587 | I.S. | 0.39793 | 14588 | I.S. | 0.39793 | 14589 | I.S. | 0.39791 |
| 14590 | I.S. | 0.39789 | 14591 | KAT6A | 0.39789 | 14592 | SPOP | 0.39789 |
| 14593 | I.S. | 0.39789 | 14594 | APC | 0.39786 | 14595 | I.S. | 0.39786 |
| 14596 | CBLB | 0.39786 | 14597 | I.S. | 0.39786 | 14598 | I.S. | 0.39786 |
| 14599 | I.S. | 0.39785 | 14600 | I.S. | 0.39781 | 14601 | I.S. | 0.39779 |
| 14602 | I.S. | 0.39779 | 14603 | I.S. | 0.39776 | 14604 | I.S. | 0.39771 |
| 14605 | BARD1 | 0.39768 | 14606 | I.S. | 0.39767 | 14607 | LRP1B | 0.39765 |
| 14608 | I.S. | 0.39765 | 14609 | MED12 | 0.39763 | 14610 | I.S. | 0.39762 |
| 14611 | KDM6A | 0.39762 | 14612 | MED12 | 0.39762 | 14613 | I.S. | 0.3976 |
| 14614 | I.S. | 0.3976 | 14615 | I.S. | 0.39757 | 14616 | I.S. | 0.39756 |
| 14617 | KMT2C | 0.39756 | 14618 | NTRK2 | 0.39754 | 14619 | CHD4 | 0.39753 |
| 14620 | I.S. | 0.39751 | 14621 | POT1 | 0.39751 | 14622 | I.S. | 0.39751 |
| 14623 | I.S. | 0.3975 | 14624 | I.S. | 0.3975 | 14625 | I.S. | 0.39748 |
| 14626 | I.S. | 0.39748 | 14627 | DOT1L | 0.39747 | 14628 | I.S. | 0.39745 |
| 14629 | CHD2 | 0.39745 | 14630 | I.S. | 0.39741 | 14631 | I.S. | 0.39739 |
| 14632 | I.S. | 0.39738 | 14633 | I.S. | 0.39738 | 14634 | I.S. | 0.39737 |
| 14635 | I.S. | 0.39735 | 14636 | ATR | 0.39732 | 14637 | I.S. | 0.39731 |
| 14638 | I.S. | 0.39729 | 14639 | I.S. | 0.39729 | 14640 | I.S. | 0.39726 |
| 14641 | I.S. | 0.39724 | 14642 | I.S. | 0.39722 | 14643 | I.S. | 0.3972 |
| 14644 | I.S. | 0.39718 | 14645 | ABL1 | 0.39715 | 14646 | I.S. | 0.39715 |
| 14647 | I.S. | 0.39712 | 14648 | MAP3K1 | 0.39711 | 14649 | I.S. | 0.39711 |
| 14650 | PIK3CG | 0.39711 | 14651 | I.S. | 0.39709 | 14652 | TERF1 | 0.39709 |
| 14653 | DNM2 | 0.39706 | 14654 | I.S. | 0.39706 | 14655 | I.S. | 0.39705 |
| 14656 | I.S. | 0.39705 | 14657 | I.S. | 0.39705 | 14658 | I.S. | 0.39703 |
| 14659 | PRKDC | 0.39703 | 14660 | PRKCI | 0.39702 | 14661 | I.S. | 0.39699 |
| 14662 | I.S. | 0.39699 | 14663 | I.S. | 0.39696 | 14664 | I.S. | 0.39695 |
| 14665 | I.S. | 0.39694 | 14666 | I.S. | 0.39692 | 14667 | SETD2 | 0.39691 |
| 14668 | I.S. | 0.39689 | 14669 | I.S. | 0.39689 | 14670 | PSTPIP1 | 0.39688 |
| 14671 | I.S. | 0.39688 | 14672 | I.S. | 0.39684 | 14673 | MEF2B | 0.39684 |
| 14674 | JAK3 | 0.3968 | 14675 | I.S. | 0.39679 | 14676 | I.S. | 0.39679 |
| 14677 | I.S. | 0.39678 | 14678 | PRKDC | 0.39674 | 14679 | AXIN2 | 0.39673 |
| 14680 | LRP1B | 0.39673 | 14681 | I.S. | 0.39673 | 14682 | PRKDC | 0.3967 |
| 14683 | I.S. | 0.39669 | 14684 | I.S. | 0.39669 | 14685 | I.S. | 0.39665 |
| 14686 | I.S. | 0.39664 | 14687 | I.S. | 0.3966 | 14688 | I.S. | 0.3966 |
| 14689 | PTEN | 0.39658 | 14690 | I.S. | 0.39656 | 14691 | I.S. | 0.39655 |
| 14692 | I.S. | 0.39655 | 14693 | I.S. | 0.39655 | 14694 | NPM1 | 0.39655 |
| 14695 | IRF2 | 0.39653 | 14696 | I.S. | 0.39653 | 14697 | I.S. | 0.39653 |
| 14698 | I.S. | 0.39651 | 14699 | GRM3 | 0.3965 | 14700 | I.S. | 0.39649 |
| 14701 | I.S. | 0.39649 | 14702 | I.S. | 0.39644 | 14703 | I.S. | 0.39644 |
| 14704 | RANBP2 | 0.39643 | 14705 | I.S. | 0.39642 | 14706 | I.S. | 0.3964 |
| 14707 | BTK | 0.3964 | 14708 | I.S. | 0.3964 | 14709 | I.S. | 0.39639 |
| 14710 | PIK3CG | 0.39638 | 14711 | I.S. | 0.39638 | 14712 | KIF23 | 0.39637 |
| 14713 | SEC23B | 0.39636 | 14714 | MAGI2 | 0.39631 | 14715 | I.S. | 0.3963 |
| 14716 | I.S. | 0.39629 | 14717 | I.S. | 0.39629 | 14718 | I.S. | 0.39623 |
| 14719 | I.S. | 0.39623 | 14720 | I.S. | 0.39622 | 14721 | I.S. | 0.39622 |
| 14722 | I.S. | 0.3962 | 14723 | I.S. | 0.3962 | 14724 | I.S. | 0.39619 |
| 14725 | I.S. | 0.39619 | 14726 | CHD4 | 0.39606 | 14727 | CYLD | 0.39604 |
| 14728 | NOTCH3 | 0.39602 | 14729 | I.S. | 0.39599 | 14730 | I.S. | 0.39596 |
| 14731 | I.S. | 0.39593 | 14732 | I.S. | 0.39593 | 14733 | XPO1 | 0.39591 |
| 14734 | I.S. | 0.3959 | 14735 | MED12 | 0.3959 | 14736 | I.S. | 0.39589 |
| 14737 | I.S. | 0.39587 | 14738 | I.S. | 0.39586 | 14739 | I.S. | 0.39584 |
| 14740 | I.S. | 0.39584 | 14741 | I.S. | 0.39581 | 14742 | I.S. | 0.3958 |
| 14743 | I.S. | 0.39579 | 14744 | I.S. | 0.39576 | 14745 | I.S. | 0.39575 |
| 14746 | KDR | 0.39574 | 14747 | TERF1 | 0.39571 | 14748 | I.S. | 0.39571 |
| 14749 | I.S. | 0.39568 | 14750 | I.S. | 0.39566 | 14751 | I.S. | 0.39566 |
| 14752 | I.S. | 0.39566 | 14753 | I.S. | 0.39563 | 14754 | I.S. | 0.3956 |
| 14755 | I.S. | 0.39559 | 14756 | I.S. | 0.39554 | 14757 | I.S. | 0.39554 |
| 14758 | I.S. | 0.39554 | 14759 | I.S. | 0.39553 | 14760 | CSF3R | 0.39551 |
| 14761 | I.S. | 0.39549 | 14762 | I.S. | 0.39549 | 14763 | WAS | 0.39549 |
| 14764 | I.S. | 0.39549 | 14765 | I.S. | 0.39549 | 14766 | I.S. | 0.39547 |
| 14767 | I.S. | 0.39546 | 14768 | I.S. | 0.39546 | 14769 | I.S. | 0.39545 |
| 14770 | I.S. | 0.39543 | 14771 | I.S. | 0.39543 | 14772 | I.S. | 0.39541 |
| 14773 | I.S. | 0.3954 | 14774 | I.S. | 0.39539 | 14775 | I.S. | 0.39538 |
| 14776 | I.S. | 0.39536 | 14777 | I.S. | 0.39533 | 14778 | I.S. | 0.3953 |
| 14779 | I.S. | 0.39528 | 14780 | I.S. | 0.39527 | 14781 | RANBP2 | 0.39527 |
| 14782 | I.S. | 0.39527 | 14783 | ERBB4 | 0.39524 | 14784 | I.S. | 0.39524 |
| 14785 | I.S. | 0.39524 | 14786 | CHD4 | 0.39522 | 14787 | I.S. | 0.39522 |
| 14788 | I.S. | 0.39518 | 14789 | KIT | 0.39516 | 14790 | I.S. | 0.39513 |
| 14791 | PLCG2 | 0.39513 | 14792 | I.S. | 0.39513 | 14793 | I.S. | 0.39513 |
| 14794 | FANCM | 0.39513 | 14795 | I.S. | 0.39512 | 14796 | I.S. | 0.39509 |
| 14797 | PALB2 | 0.39509 | 14798 | I.S. | 0.39508 | 14799 | I.S. | 0.39506 |
| 14800 | I.S. | 0.39503 | 14801 | I.S. | 0.39502 | 14802 | I.S. | 0.395 |
| 14803 | I.S. | 0.39496 | 14804 | I.S. | 0.39495 | 14805 | I.S. | 0.39495 |
| 14806 | I.S. | 0.39494 | 14807 | I.S. | 0.39493 | 14808 | I.S. | 0.39492 |
| 14809 | I.S. | 0.39492 | 14810 | I.S. | 0.39488 | 14811 | I.S. | 0.39487 |
| 14812 | FAS | 0.39486 | 14813 | I.S. | 0.39482 | 14814 | I.S. | 0.39477 |
| 14815 | I.S. | 0.39476 | 14816 | I.S. | 0.39473 | 14817 | I.S. | 0.39472 |
| 14818 | I.S. | 0.39468 | 14819 | I.S. | 0.39468 | 14820 | I.S. | 0.39468 |
| 14821 | I.S. | 0.39467 | 14822 | I.S. | 0.39467 | 14823 | I.S. | 0.39466 |
| 14824 | I.S. | 0.39465 | 14825 | I.S. | 0.39465 | 14826 | I.S. | 0.39462 |
| 14827 | BCORL1 | 0.39462 | 14828 | PRKDC | 0.39457 | 14829 | I.S. | 0.39457 |
| 14830 | I.S. | 0.39457 | 14831 | I.S. | 0.39456 | 14832 | RANBP2 | 0.39456 |
| 14833 | BCR | 0.39456 | 14834 | I.S. | 0.39454 | 14835 | I.S. | 0.39452 |
| 14836 | KAT6A | 0.39451 | 14837 | I.S. | 0.39451 | 14838 | I.S. | 0.39451 |
| 14839 | I.S. | 0.39447 | 14840 | SMAD3 | 0.39447 | 14841 | I.S. | 0.39445 |
| 14842 | I.S. | 0.39445 | 14843 | I.S. | 0.39443 | 14844 | I.S. | 0.39442 |
| 14845 | I.S. | 0.39439 | 14846 | I.S. | 0.39439 | 14847 | I.S. | 0.39438 |
| 14848 | I.S. | 0.39438 | 14849 | I.S. | 0.39438 | 14850 | I.S. | 0.39435 |
| 14851 | NTRK1 | 0.39433 | 14852 | I.S. | 0.39432 | 14853 | I.S. | 0.39432 |
| 14854 | PBRM1 | 0.39429 | 14855 | I.S. | 0.39429 | 14856 | I.S. | 0.39429 |
| 14857 | I.S. | 0.39428 | 14858 | I.S. | 0.39428 | 14859 | I.S. | 0.39427 |
| 14860 | I.S. | 0.39427 | 14861 | I.S. | 0.39426 | 14862 | I.S. | 0.39425 |
| 14863 | I.S. | 0.39424 | 14864 | I.S. | 0.39424 | 14865 | DDX41 | 0.39424 |
| 14866 | I.S. | 0.39424 | 14867 | I.S. | 0.39423 | 14868 | BCORL1 | 0.39421 |
| 14869 | I.S. | 0.3942 | 14870 | I.S. | 0.39418 | 14871 | I.S. | 0.39415 |
| 14872 | I.S. | 0.39414 | 14873 | I.S. | 0.39412 | 14874 | I.S. | 0.39411 |
| 14875 | I.S. | 0.39407 | 14876 | I.S. | 0.39406 | 14877 | I.S. | 0.39406 |
| 14878 | I.S. | 0.39406 | 14879 | CHD4 | 0.39406 | 14880 | I.S. | 0.39406 |
| 14881 | I.S. | 0.39403 | 14882 | I.S. | 0.39403 | 14883 | I.S. | 0.39402 |
| 14884 | I.S. | 0.39401 | 14885 | NF2 | 0.394 | 14886 | I.S. | 0.39398 |
| 14887 | I.S. | 0.39395 | 14888 | I.S. | 0.39395 | 14889 | I.S. | 0.39394 |
| 14890 | I.S. | 0.39394 | 14891 | PRKDC | 0.39391 | 14892 | CHD2 | 0.3939 |
| 14893 | I.S. | 0.3939 | 14894 | SUZ12 | 0.3939 | 14895 | I.S. | 0.39389 |
| 14896 | I.S. | 0.39388 | 14897 | LRP1B | 0.39382 | 14898 | I.S. | 0.39382 |
| 14899 | I.S. | 0.39382 | 14900 | I.S. | 0.39379 | 14901 | I.S. | 0.39379 |
| 14902 | I.S. | 0.39378 | 14903 | I.S. | 0.39378 | 14904 | I.S. | 0.39377 |
| 14905 | I.S. | 0.39376 | 14906 | ERBB4 | 0.39373 | 14907 | I.S. | 0.39372 |
| 14908 | PRKDC | 0.3937 | 14909 | I.S. | 0.3937 | 14910 | ATRX | 0.39368 |
| 14911 | — | 0.39368 | 14912 | MLH1 | 0.39368 | 14913 | I.S. | 0.39365 |
| 14914 | I.S. | 0.39363 | 14915 | I.S. | 0.39361 | 14916 | I.S. | 0.39361 |
| 14917 | I.S. | 0.3936 | 14918 | I.S. | 0.3936 | 14919 | FAS | 0.3936 |
| 14920 | I.S. | 0.39359 | 14921 | I.S. | 0.39357 | 14922 | I.S. | 0.39357 |
| 14923 | I.S. | 0.39355 | 14924 | I.S. | 0.39355 | 14925 | KDM6A | 0.39355 |
| 14926 | I.S. | 0.39352 | 14927 | I.S. | 0.39352 | 14928 | CDAN1 | 0.39352 |
| 14929 | I.S. | 0.39352 | 14930 | I.S. | 0.39351 | 14931 | I.S. | 0.39349 |
| 14932 | I.S. | 0.39349 | 14933 | I.S. | 0.39348 | 14934 | I.S. | 0.39346 |
| 14935 | DOT1L | 0.39343 | 14936 | I.S. | 0.39341 | 14937 | ATM | 0.39341 |
| 14938 | I.S. | 0.39341 | 14939 | I.S. | 0.39338 | 14940 | I.S. | 0.39338 |
| 14941 | I.S. | 0.39337 | 14942 | I.S. | 0.39336 | 14943 | I.S. | 0.39335 |
| 14944 | I.S. | 0.39335 | 14945 | I.S. | 0.39335 | 14946 | I.S. | 0.39335 |
| 14947 | I.S. | 0.39332 | 14948 | I.S. | 0.39331 | 14949 | I.S. | 0.39329 |
| 14950 | POT1 | 0.39326 | 14951 | I.S. | 0.39322 | 14952 | I.S. | 0.39322 |
| 14953 | I.S. | 0.3932 | 14954 | I.S. | 0.39319 | 14955 | I.S. | 0.39314 |
| 14956 | I.S. | 0.39313 | 14957 | PRKDC | 0.39311 | 14958 | SMAD3 | 0.39311 |
| 14959 | I.S. | 0.39311 | 14960 | I.S. | 0.39311 | 14961 | I.S. | 0.3931 |
| 14962 | KDM5C | 0.39302 | 14963 | RICTOR | 0.39301 | 14964 | I.S. | 0.393 |
| 14965 | I.S. | 0.39299 | 14966 | I.S. | 0.39299 | 14967 | I.S. | 0.39293 |
| 14968 | ATRX | 0.3929 | 14969 | I.S. | 0.39288 | 14970 | I.S. | 0.39285 |
| 14971 | I.S. | 0.39281 | 14972 | I.S. | 0.3928 | 14973 | ATR | 0.39279 |
| 14974 | I.S. | 0.39278 | 14975 | KDM6A | 0.39277 | 14976 | I.S. | 0.39276 |
| 14977 | POT1 | 0.39275 | 14978 | I.S. | 0.39275 | 14979 | I.S. | 0.39275 |
| 14980 | I.S. | 0.39275 | 14981 | I.S. | 0.39275 | 14982 | I.S. | 0.39273 |
| 14983 | I.S. | 0.39272 | 14984 | I.S. | 0.39272 | 14985 | I.S. | 0.39272 |
| 14986 | I.S. | 0.3927 | 14987 | ATRX | 0.39268 | 14988 | I.S. | 0.39266 |
| 14989 | I.S. | 0.39266 | 14990 | I.S. | 0.39266 | 14991 | I.S. | 0.39264 |
| 14992 | — | 0.39263 | 14993 | I.S. | 0.3926 | 14994 | I.S. | 0.39259 |
| 14995 | I.S. | 0.39257 | 14996 | I.S. | 0.39257 | 14997 | I.S. | 0.39255 |
| 14998 | I.S. | 0.39254 | 14999 | I.S. | 0.39254 | 15000 | I.S. | 0.39254 |
| 15001 | I.S. | 0.39254 | 15002 | STAG2 | 0.39252 | 15003 | I.S. | 0.39251 |
| 15004 | I.S. | 0.3925 | 15005 | I.S. | 0.39245 | 15006 | I.S. | 0.39245 |
| 15007 | NOTCH1 | 0.39245 | 15008 | I.S. | 0.39244 | 15009 | I.S. | 0.39242 |
| 15010 | I.S. | 0.39242 | 15011 | I.S. | 0.39238 | 15012 | I.S. | 0.39237 |
| 15013 | I.S. | 0.39236 | 15014 | CUX1 | 0.39234 | 15015 | I.S. | 0.39233 |
| 15016 | I.S. | 0.39233 | 15017 | I.S. | 0.39233 | 15018 | I.S. | 0.3923 |
| 15019 | KMT2A | 0.39229 | 15020 | I.S. | 0.39229 | 15021 | I.S. | 0.39227 |
| 15022 | I.S. | 0.39225 | 15023 | MVK | 0.39222 | 15024 | I.S. | 0.39222 |
| 15025 | I.S. | 0.39222 | 15026 | I.S. | 0.39221 | 15027 | I.S. | 0.39221 |
| 15028 | I.S. | 0.39221 | 15029 | I.S. | 0.39219 | 15030 | TCIRG1 | 0.39216 |
| 15031 | I.S. | 0.39212 | 15032 | I.S. | 0.3921 | 15033 | I.S. | 0.39209 |
| 15034 | IRAIN, IGF1R | 0.39208 | 15035 | I.S. | 0.39207 | 15036 | I.S. | 0.39204 |
| 15037 | PMS2 | 0.39204 | 15038 | I.S. | 0.39204 | 15039 | I.S. | 0.39201 |
| 15040 | I.S. | 0.392 | 15041 | I.S. | 0.39198 | 15042 | I.S. | 0.39197 |
| 15043 | I.S. | 0.39192 | 15044 | I.S. | 0.39186 | 15045 | ATM | 0.39184 |
| 15046 | ACVR1B | 0.39183 | 15047 | I.S. | 0.39182 | 15048 | I.S. | 0.39182 |
| 15049 | I.S. | 0.3918 | 15050 | I.S. | 0.3918 | 15051 | I.S. | 0.3918 |
| 15052 | I.S. | 0.39179 | 15053 | I.S. | 0.39179 | 15054 | PHF6 | 0.39178 |
| 15055 | KIF23 | 0.39177 | 15056 | PRKDC | 0.39177 | 15057 | I.S. | 0.39173 |
| 15058 | I.S. | 0.39171 | 15059 | I.S. | 0.3917 | 15060 | I.S. | 0.39165 |
| 15061 | I.S. | 0.39165 | 15062 | I.S. | 0.39165 | 15063 | I.S. | 0.39164 |
| 15064 | I.S. | 0.3916 | 15065 | I.S. | 0.3916 | 15066 | I.S. | 0.39156 |
| 15067 | I.S. | 0.39155 | 15068 | I.S. | 0.39153 | 15069 | I.S. | 0.39153 |
| 15070 | I.S. | 0.39153 | 15071 | I.S. | 0.39153 | 15072 | I.S. | 0.39152 |
| 15073 | I.S. | 0.39152 | 15074 | I.S. | 0.3915 | 15075 | NUP93 | 0.39149 |
| 15076 | I.S. | 0.39145 | 15077 | AURKB | 0.39144 | 15078 | KIT | 0.39144 |
| 15079 | GNAQ | 0.39141 | 15080 | I.S. | 0.39141 | 15081 | I.S. | 0.39141 |
| 15082 | I.S. | 0.39139 | 15083 | I.S. | 0.39138 | 15084 | XRCC2 | 0.39135 |
| 15085 | I.S. | 0.39135 | 15086 | I.S. | 0.3913 | 15087 | I.S. | 0.3913 |
| 15088 | NOTCH1 | 0.3913 | 15089 | SF3B1 | 0.3913 | 15090 | KMT2A | 0.39129 |
| 15091 | I.S. | 0.39129 | 15092 | I.S. | 0.39129 | 15093 | I.S. | 0.39126 |
| 15094 | I.S. | 0.39121 | 15095 | ADGRA2 | 0.3912 | 15096 | I.S. | 0.39118 |
| 15097 | I.S. | 0.39117 | 15098 | EPCAM | 0.39114 | 15099 | RICTOR | 0.39114 |
| 15100 | I.S. | 0.39114 | 15101 | ATRX | 0.39114 | 15102 | I.S. | 0.39114 |
| 15103 | I.S. | 0.39112 | 15104 | I.S. | 0.39112 | 15105 | TERF1 | 0.39109 |
| 15106 | CYLD | 0.391 | 15107 | CUX1 | 0.39099 | 15108 | I.S. | 0.39097 |
| 15109 | SPEN | 0.39097 | 15110 | I.S. | 0.39097 | 15111 | I.S. | 0.39097 |
| 15112 | I.S. | 0.39097 | 15113 | U2AF2 | 0.39097 | 15114 | I.S. | 0.39096 |
| 15115 | I.S. | 0.39092 | 15116 | DICER1 | 0.39091 | 15117 | EPHA5 | 0.39089 |
| 15118 | I.S. | 0.39087 | 15119 | I.S. | 0.39085 | 15120 | I.S. | 0.39084 |
| 15121 | I.S. | 0.39082 | 15122 | I.S. | 0.39081 | 15123 | CHD2 | 0.39079 |
| 15124 | RAD51 | 0.39078 | 15125 | I.S. | 0.39076 | 15126 | I.S. | 0.39076 |
| 15127 | I.S. | 0.39076 | 15128 | PDK1 | 0.39073 | 15129 | I.S. | 0.39073 |
| 15130 | MED12 | 0.39067 | 15131 | NOTCH3 | 0.39067 | 15132 | NF2 | 0.39065 |
| 15133 | I.S. | 0.39064 | 15134 | CDK6 | 0.39064 | 15135 | I.S. | 0.39064 |
| 15136 | I.S. | 0.39061 | 15137 | GEN1 | 0.39058 | 15138 | ATM | 0.39057 |
| 15139 | I.S. | 0.39054 | 15140 | I.S. | 0.39053 | 15141 | NF2 | 0.39053 |
| 15142 | ANKRD26 | 0.39049 | 15143 | I.S. | 0.39045 | 15144 | I.S. | 0.39043 |
| 15145 | I.S. | 0.39043 | 15146 | NOTCH1 | 0.39043 | 15147 | ARID2 | 0.39043 |
| 15148 | I.S. | 0.3904 | 15149 | I.S. | 0.3904 | 15150 | I.S. | 0.39037 |
| 15151 | I.S. | 0.39037 | 15152 | I.S. | 0.39035 | 15153 | I.S. | 0.39032 |
| 15154 | TSC1 | 0.39032 | 15155 | I.S. | 0.39027 | 15156 | FANCC | 0.39027 |
| 15157 | I.S. | 0.39026 | 15158 | BLM | 0.39026 | 15159 | I.S. | 0.39025 |
| 15160 | I.S. | 0.39023 | 15161 | H3F3AP4 | 0.39023 | 15162 | KMT2B | 0.39023 |
| | | | | H3F3A, | | | | |
| 15163 | MRE11 | 0.39022 | 15164 | I.S. | 0.3902 | 15165 | IRF2 | 0.39019 |
| 15166 | I.S. | 0.39017 | 15167 | ABL1 | 0.39017 | 15168 | AURKB | 0.39016 |
| 15169 | I.S. | 0.39015 | 15170 | I.S. | 0.39013 | 15171 | BRCA1 | 0.3901 |
| 15172 | I.S. | 0.3901 | 15173 | I.S. | 0.3901 | 15174 | BCL2 | 0.39008 |
| 15175 | RANBP2 | 0.39007 | 15176 | I.S. | 0.39005 | 15177 | CCND1 | 0.39005 |
| 15178 | MSH2 | 0.39002 | 15179 | I.S. | 0.39002 | 15180 | I.S. | 0.38999 |
| 15181 | I.S. | 0.38999 | 15182 | BCR | 0.38996 | 15183 | KDM6A | 0.38993 |
| 15184 | I.S. | 0.38993 | 15185 | CYLD | 0.38987 | 15186 | I.S. | 0.38984 |
| 15187 | I.S. | 0.38984 | 15188 | XPO1 | 0.38983 | 15189 | I.S. | 0.38982 |
| 15190 | I.S. | 0.3898 | 15191 | I.S. | 0.38978 | 15192 | I.S. | 0.38976 |
| 15193 | I.S. | 0.38975 | 15194 | I.S. | 0.38975 | 15195 | I.S. | 0.38975 |
| 15196 | ATRX | 0.38969 | 15197 | I.S. | 0.38968 | 15198 | DNMT3A | 0.38966 |
| 15199 | I.S. | 0.38966 | 15200 | I.S. | 0.38966 | 15201 | I.S. | 0.38966 |
| 15202 | I.S. | 0.38959 | 15203 | I.S. | 0.38958 | 15204 | I.S. | 0.38957 |
| 15205 | I.S. | 0.38957 | 15206 | I.S. | 0.38956 | 15207 | I.S. | 0.38953 |
| 15208 | I.S. | 0.38952 | 15209 | I.S. | 0.3895 | 15210 | I.S. | 0.38948 |
| 15211 | I.S. | 0.38947 | 15212 | I.S. | 0.38946 | 15213 | I.S. | 0.38945 |
| 15214 | I.S. | 0.38943 | 15215 | I.S. | 0.38939 | 15216 | TSC2 | 0.38939 |
| 15217 | I.S. | 0.38938 | 15218 | EPHB1 | 0.38937 | 15219 | CBL | 0.38937 |
| 15220 | I.S. | 0.38933 | 15221 | I.S. | 0.38933 | 15222 | I.S. | 0.38933 |
| 15223 | I.S. | 0.38933 | 15224 | I.S. | 0.3893 | 15225 | I.S. | 0.38929 |
| 15226 | ERBB4 | 0.38925 | 15227 | I.S. | 0.38924 | 15228 | CHEK2 | 0.38924 |
| 15229 | MED12 | 0.38924 | 15230 | SBF2 | 0.38924 | 15231 | PRKDC | 0.38922 |
| 15232 | I.S. | 0.38922 | 15233 | I.S. | 0.38922 | 15234 | I.S. | 0.3892 |
| 15235 | I.S. | 0.3892 | 15236 | I.S. | 0.38919 | 15237 | I.S. | 0.38918 |
| 15238 | I.S. | 0.38916 | 15239 | I.S. | 0.38914 | 15240 | LRP1B | 0.38913 |
| 15241 | I.S. | 0.38913 | 15242 | I.S. | 0.38912 | 15243 | I.S. | 0.38908 |
| 15244 | TP53 | 0.38906 | 15245 | I.S. | 0.38903 | 15246 | I.S. | 0.38903 |
| 15247 | I.S. | 0.38903 | 15248 | I.S. | 0.38901 | 15249 | I.S. | 0.38901 |
| 15250 | I.S. | 0.389 | 15251 | I.S. | 0.389 | 15252 | I.S. | 0.38899 |
| 15253 | I.S. | 0.38897 | 15254 | FGF19 | 0.38897 | 15255 | I.S. | 0.38897 |
| 15256 | I.S. | 0.38895 | 15257 | I.S. | 0.38895 | 15258 | I.S. | 0.38895 |
| 15259 | I.S. | 0.38895 | 15260 | I.S. | 0.38894 | 15261 | I.S. | 0.38892 |
| 15262 | I.S. | 0.38892 | 15263 | I.S. | 0.38892 | 15264 | I.S. | 0.38892 |
| 15265 | I.S. | 0.38892 | 15266 | EPHA5 | 0.38889 | 15267 | I.S. | 0.38889 |
| 15268 | I.S. | 0.38887 | 15269 | I.S. | 0.38886 | 15270 | I.S. | 0.38886 |
| 15271 | PRKDC | 0.38877 | 15272 | I.S. | 0.38877 | 15273 | I.S. | 0.38876 |
| 15274 | I.S. | 0.38873 | 15275 | I.S. | 0.38873 | 15276 | I.S. | 0.38872 |
| 15277 | MED12 | 0.38871 | 15278 | STAT4 | 0.38871 | 15279 | I.S. | 0.38871 |
| 15280 | I.S. | 0.38868 | 15281 | ANKRD26 | 0.38868 | 15282 | I.S. | 0.38868 |
| 15283 | I.S. | 0.38867 | 15284 | — | 0.38866 | 15285 | I.S. | 0.38864 |
| 15286 | I.S. | 0.38862 | 15287 | I.S. | 0.38862 | 15288 | I.S. | 0.38862 |
| 15289 | I.S. | 0.38862 | 15290 | I.S. | 0.38862 | 15291 | I.S. | 0.38861 |
| 15292 | I.S. | 0.38859 | 15293 | I.S. | 0.38859 | 15294 | I.S. | 0.38856 |
| 15295 | I.S. | 0.38856 | 15296 | I.S. | 0.38856 | 15297 | NOTCH1 | 0.38855 |
| 15298 | I.S. | 0.38854 | 15299 | SLIT2 | 0.38854 | 15300 | I.S. | 0.38851 |
| 15301 | FANCI | 0.38851 | 15302 | I.S. | 0.38851 | 15303 | I.S. | 0.38851 |
| 15304 | I.S. | 0.3885 | 15305 | I.S. | 0.3885 | 15306 | I.S. | 0.3885 |
| 15307 | I.S. | 0.38849 | 15308 | I.S. | 0.38847 | 15309 | I.S. | 0.38845 |
| 15310 | I.S. | 0.38845 | 15311 | I.S. | 0.38845 | 15312 | I.S. | 0.38843 |
| 15313 | MAGI2 | 0.38841 | 15314 | I.S. | 0.38838 | 15315 | I.S. | 0.38837 |
| 15316 | I.S. | 0.3883 | 15317 | MAGI2 | 0.38826 | 15318 | I.S. | 0.38826 |
| 15319 | I.S. | 0.38826 | 15320 | I.S. | 0.38826 | 15321 | I.S. | 0.38823 |
| 15322 | GRM3 | 0.38821 | 15323 | I.S. | 0.38821 | 15324 | I.S. | 0.3882 |
| 15325 | I.S. | 0.3882 | 15326 | I.S. | 0.38818 | 15327 | I.S. | 0.38818 |
| 15328 | I.S. | 0.38818 | 15329 | I.S. | 0.38817 | 15330 | I.S. | 0.38816 |
| 15331 | TSC1 | 0.38815 | 15332 | I.S. | 0.38812 | 15333 | I.S. | 0.38811 |
| 15334 | GRM3 | 0.3881 | 15335 | I.S. | 0.38809 | 15336 | I.S. | 0.38809 |
| 15337 | I.S. | 0.38808 | 15338 | I.S. | 0.38806 | 15339 | I.S. | 0.38806 |
| 15340 | I.S. | 0.38806 | 15341 | I.S. | 0.38806 | 15342 | TAL1 | 0.38806 |
| 15343 | I.S. | 0.38805 | 15344 | I.S. | 0.38805 | 15345 | I.S. | 0.38805 |
| 15346 | PBRM1 | 0.38803 | 15347 | ANKRD26 | 0.38802 | 15348 | I.S. | 0.38802 |
| 15349 | I.S. | 0.38802 | 15350 | IL2RG | 0.388 | 15351 | I.S. | 0.38799 |
| 15352 | I.S. | 0.38798 | 15353 | I.S. | 0.38797 | 15354 | I.S. | 0.38797 |
| 15355 | I.S. | 0.38795 | 15356 | I.S. | 0.38794 | 15357 | MSH2 | 0.38794 |
| 15358 | I.S. | 0.38794 | 15359 | I.S. | 0.38794 | 15360 | I.S. | 0.38792 |
| 15361 | I.S. | 0.38791 | 15362 | FANCB | 0.3879 | 15363 | I.S. | 0.38788 |
| 15364 | I.S. | 0.38785 | 15365 | I.S. | 0.38785 | 15366 | I.S. | 0.38785 |
| 15367 | I.S. | 0.38785 | 15368 | I.S. | 0.38785 | 15369 | PAK3 | 0.38782 |
| 15370 | I.S. | 0.38782 | 15371 | I.S. | 0.38782 | 15372 | I.S. | 0.38782 |
| 15373 | I.S. | 0.3878 | 15374 | EPHA7 | 0.38779 | 15375 | I.S. | 0.38779 |
| 15376 | I.S. | 0.38778 | 15377 | I.S. | 0.38777 | 15378 | I.S. | 0.38777 |
| 15379 | I.S. | 0.38776 | 15380 | I.S. | 0.38776 | 15381 | ANKRD26 | 0.38774 |
| 15382 | I.S. | 0.38774 | 15383 | I.S. | 0.38773 | 15384 | I.S. | 0.38773 |
| 15385 | I.S. | 0.38772 | 15386 | I.S. | 0.38772 | 15387 | I.S. | 0.38772 |
| 15388 | I.S. | 0.38771 | 15389 | I.S. | 0.3877 | 15390 | I.S. | 0.38769 |
| 15391 | I.S. | 0.38767 | 15392 | I.S. | 0.38767 | 15393 | I.S. | 0.38766 |
| 15394 | MTOR | 0.38764 | 15395 | I.S. | 0.38764 | 15396 | I.S. | 0.38764 |
| 15397 | I.S. | 0.38764 | 15398 | I.S. | 0.38764 | 15399 | I.S. | 0.38761 |
| 15400 | I.S. | 0.38761 | 15401 | I.S. | 0.38761 | 15402 | I.S. | 0.38761 |
| 15403 | FAT1 | 0.38758 | 15404 | I.S. | 0.38758 | 15405 | I.S. | 0.38757 |
| 15406 | I.S. | 0.38757 | 15407 | I.S. | 0.38756 | 15408 | I.S. | 0.38753 |
| 15409 | I.S. | 0.38753 | 15410 | I.S. | 0.38749 | 15411 | BTG1 | 0.38748 |
| 15412 | I.S. | 0.38746 | 15413 | I.S. | 0.38746 | 15414 | APC | 0.38744 |
| 15415 | ATRX | 0.38744 | 15416 | I.S. | 0.38743 | 15417 | I.S. | 0.38743 |
| 15418 | I.S. | 0.38743 | 15419 | I.S. | 0.38741 | 15420 | I.S. | 0.3874 |
| 15421 | I.S. | 0.3874 | 15422 | I.S. | 0.38736 | 15423 | LRP1B | 0.38731 |
| 15424 | I.S. | 0.38731 | 15425 | I.S. | 0.38729 | 15426 | I.S. | 0.38728 |
| 15427 | I.S. | 0.38728 | 15428 | I.S. | 0.38728 | 15429 | I.S. | 0.38727 |
| 15430 | PRKDC | 0.38726 | 15431 | I.S. | 0.38725 | 15432 | I.S. | 0.38723 |
| 15433 | I.S. | 0.38723 | 15434 | I.S. | 0.38722 | 15435 | I.S. | 0.38722 |
| 15436 | I.S. | 0.38722 | 15437 | RB1 | 0.38722 | 15438 | I.S. | 0.38719 |
| 15439 | I.S. | 0.38719 | 15440 | I.S. | 0.38719 | 15441 | I.S. | 0.38716 |
| 15442 | I.S. | 0.38714 | 15443 | I.S. | 0.38714 | 15444 | I.S. | 0.38711 |
| 15445 | I.S. | 0.38711 | 15446 | SDHB | 0.38708 | 15447 | I.S. | 0.38708 |
| 15448 | I.S. | 0.38699 | 15449 | EPCAM | 0.38699 | 15450 | KMT2C | 0.38698 |
| 15451 | I.S. | 0.38698 | 15452 | I.S. | 0.38698 | 15453 | ATRX | 0.38696 |
| 15454 | I.S. | 0.38695 | 15455 | I.S. | 0.38693 | 15456 | PTCH1 | 0.3869 |
| 15457 | I.S. | 0.3869 | 15458 | I.S. | 0.38688 | 15459 | MAP3K1 | 0.38687 |
| 15460 | I.S. | 0.38686 | 15461 | I.S. | 0.38681 | 15462 | I.S. | 0.38681 |
| 15463 | I.S. | 0.38681 | 15464 | I.S. | 0.38681 | 15465 | I.S. | 0.3868 |
| 15466 | STAT3 | 0.3868 | 15467 | I.S. | 0.3868 | 15468 | I.S. | 0.38678 |
| 15469 | I.S. | 0.38678 | 15470 | I.S. | 0.38678 | 15471 | I.S. | 0.38675 |
| 15472 | I.S. | 0.3867 | 15473 | I.S. | 0.3867 | 15474 | I.S. | 0.3867 |
| 15475 | I.S. | 0.38669 | 15476 | I.S. | 0.38665 | 15477 | I.S. | 0.38665 |
| 15478 | I.S. | 0.38665 | 15479 | I.S. | 0.38663 | 15480 | I.S. | 0.38663 |
| 15481 | I.S. | 0.38663 | 15482 | I.S. | 0.3866 | 15483 | I.S. | 0.3866 |
| 15484 | NOTCH1 | 0.3866 | 15485 | I.S. | 0.3866 | 15486 | I.S. | 0.38658 |
| 15487 | CRLF2 | 0.38657 | 15488 | I.S. | 0.38657 | 15489 | ARID2 | 0.38657 |
| 15490 | INPP4B | 0.38651 | 15491 | I.S. | 0.38651 | 15492 | I.S. | 0.38651 |
| 15493 | RANBP2 | 0.3865 | 15494 | I.S. | 0.38649 | 15495 | FANCL | 0.38645 |
| 15496 | I.S. | 0.38645 | 15497 | I.S. | 0.38645 | 15498 | I.S. | 0.38644 |
| 15499 | MAP3K1 | 0.38642 | 15500 | I.S. | 0.38637 | 15501 | I.S. | 0.38637 |
| 15502 | I.S. | 0.38634 | 15503 | I.S. | 0.38633 | 15504 | I.S. | 0.38631 |
| 15505 | I.S. | 0.38627 | 15506 | I.S. | 0.38624 | 15507 | I.S. | 0.38624 |
| 15508 | I.S. | 0.38624 | 15509 | I.S. | 0.38622 | 15510 | RAD54L | 0.38621 |
| 15511 | I.S. | 0.38621 | 15512 | I.S. | 0.38616 | 15513 | I.S. | 0.38616 |
| 15514 | I.S. | 0.38616 | 15515 | I.S. | 0.38616 | 15516 | I.S. | 0.38615 |
| 15517 | CUX1 | 0.38615 | 15518 | PIK3CG | 0.38613 | 15519 | I.S. | 0.38613 |
| 15520 | I.S. | 0.38613 | 15521 | I.S. | 0.38612 | 15522 | I.S. | 0.38612 |
| 15523 | I.S. | 0.38611 | 15524 | I.S. | 0.3861 | 15525 | I.S. | 0.3861 |
| 15526 | I.S. | 0.38609 | 15527 | I.S. | 0.38607 | 15528 | I.S. | 0.38604 |
| 15529 | NBN | 0.38603 | 15530 | PIK3CB | 0.38602 | 15531 | I.S. | 0.38601 |
| 15532 | I.S. | 0.38601 | 15533 | XPO1 | 0.38601 | 15534 | I.S. | 0.38599 |
| 15535 | I.S. | 0.38598 | 15536 | MET | 0.38595 | 15537 | I.S. | 0.38595 |
| 15538 | I.S. | 0.38595 | 15539 | I.S. | 0.38595 | 15540 | I.S. | 0.38594 |
| 15541 | I.S. | 0.38594 | 15542 | I.S. | 0.38594 | 15543 | I.S. | 0.38592 |
| 15544 | I.S. | 0.38589 | 15545 | I.S. | 0.38588 | 15546 | ETV6 | 0.38586 |
| 15547 | NFKBIA | 0.38585 | 15548 | BAP1 | 0.38583 | 15549 | I.S. | 0.38583 |
| 15550 | I.S. | 0.38583 | 15551 | I.S. | 0.38582 | 15552 | FANCM | 0.3858 |
| 15553 | I.S. | 0.3858 | 15554 | I.S. | 0.38577 | 15555 | I.S. | 0.38576 |
| 15556 | I.S. | 0.38576 | 15557 | XPO1 | 0.38576 | 15558 | EPAS1 | 0.38568 |
| 15559 | I.S. | 0.38568 | 15560 | PTCH1 | 0.38567 | 15561 | FLT4 | 0.38567 |
| 15562 | I.S. | 0.38566 | 15563 | STAT3 | 0.38566 | 15564 | I.S. | 0.38565 |
| 15565 | I.S. | 0.38563 | 15566 | I.S. | 0.38562 | 15567 | I.S. | 0.38562 |
| 15568 | I.S. | 0.3856 | 15569 | I.S. | 0.38559 | 15570 | I.S. | 0.38558 |
| 15571 | I.S. | 0.38558 | 15572 | I.S. | 0.38558 | 15573 | BCOR | 0.38556 |
| 15574 | PIK3R1 | 0.38553 | 15575 | I.S. | 0.3855 | 15576 | I.S. | 0.3855 |
| 15577 | I.S. | 0.3855 | 15578 | PHF6 | 0.38548 | 15579 | I.S. | 0.38548 |
| 15580 | FANCC | 0.38547 | 15581 | I.S. | 0.38547 | 15582 | I.S. | 0.38541 |
| 15583 | I.S. | 0.3854 | 15584 | I.S. | 0.3854 | 15585 | I.S. | 0.38539 |
| 15586 | I.S. | 0.38539 | 15587 | MAGI2 | 0.38538 | 15588 | I.S. | 0.38537 |
| 15589 | I.S. | 0.38536 | 15590 | I.S. | 0.38536 | 15591 | I.S. | 0.38533 |
| 15592 | I.S. | 0.38531 | 15593 | I.S. | 0.3853 | 15594 | I.S. | 0.38529 |
| 15595 | I.S. | 0.38529 | 15596 | I.S. | 0.38527 | 15597 | I.S. | 0.38527 |
| 15598 | I.S. | 0.38525 | 15599 | I.S. | 0.38522 | 15600 | I.S. | 0.38521 |
| 15601 | IRF4 | 0.38521 | 15602 | I.S. | 0.3852 | 15603 | I.S. | 0.3852 |
| 15604 | I.S. | 0.38517 | 15605 | NSD2 | 0.38514 | 15606 | MED12 | 0.38509 |
| 15607 | I.S. | 0.38509 | 15608 | I.S. | 0.38509 | 15609 | I.S. | 0.38508 |
| 15610 | SMO | 0.38507 | 15611 | I.S. | 0.38506 | 15612 | I.S. | 0.38506 |
| 15613 | SETD2 | 0.38505 | 15614 | I.S. | 0.38504 | 15615 | NOTCH3 | 0.38503 |
| 15616 | I.S. | 0.38503 | 15617 | I.S. | 0.385 | 15618 | I.S. | 0.38497 |
| 15619 | I.S. | 0.38496 | 15620 | I.S. | 0.38493 | 15621 | I.S. | 0.38491 |
| 15622 | FUBP1 | 0.38489 | 15623 | I.S. | 0.38488 | 15624 | I.S. | 0.38488 |
| 15625 | DOT1L | 0.38487 | 15626 | PRKDC | 0.38485 | 15627 | I.S. | 0.38484 |
| 15628 | I.S. | 0.38484 | 15629 | I.S. | 0.38482 | 15630 | I.S. | 0.38481 |
| 15631 | ATRX | 0.38479 | 15632 | I.S. | 0.38476 | 15633 | I.S. | 0.38476 |
| 15634 | I.S. | 0.38476 | 15635 | I.S. | 0.38476 | 15636 | I.S. | 0.38476 |
| 15637 | BTK | 0.38476 | 15638 | I.S. | 0.38475 | 15639 | I.S. | 0.38473 |
| 15640 | I.S. | 0.38473 | 15641 | I.S. | 0.38473 | 15642 | I.S. | 0.38472 |
| 15643 | I.S. | 0.38471 | 15644 | I.S. | 0.38471 | 15645 | I.S. | 0.38471 |
| 15646 | I.S. | 0.3847 | 15647 | I.S. | 0.38469 | 15648 | I.S. | 0.38469 |
| 15649 | I.S. | 0.38466 | 15650 | I.S. | 0.38464 | 15651 | I.S. | 0.38464 |
| 15652 | I.S. | 0.38463 | 15653 | I.S. | 0.38463 | 15654 | I.S. | 0.3846 |
| 15655 | I.S. | 0.38458 | 15656 | I.S. | 0.38455 | 15657 | I.S. | 0.38454 |
| 15658 | I.S. | 0.38453 | 15659 | CHD2 | 0.38448 | 15660 | I.S. | 0.38446 |
| 15661 | I.S. | 0.38446 | 15662 | I.S. | 0.38443 | 15663 | I.S. | 0.38443 |
| 15664 | I.S. | 0.38443 | 15665 | NPM1 | 0.3844 | 15666 | ANKRD26 | 0.38438 |
| 15667 | CREBBP | 0.38437 | 15668 | I.S. | 0.38435 | 15669 | I.S. | 0.38435 |
| 15670 | I.S. | 0.38435 | 15671 | I.S. | 0.38432 | 15672 | I.S. | 0.38432 |
| 15673 | I.S. | 0.38431 | 15674 | I.S. | 0.38428 | 15675 | I.S. | 0.38428 |
| 15676 | I.S. | 0.38427 | 15677 | I.S. | 0.38426 | 15678 | SMARCA4 | 0.38426 |
| 15679 | BCOR | 0.38425 | 15680 | I.S. | 0.38425 | 15681 | PDGFRA | 0.38423 |
| 15682 | PAX5 | 0.38423 | 15683 | I.S. | 0.38422 | 15684 | I.S. | 0.38422 |
| 15685 | I.S. | 0.38421 | 15686 | MTOR | 0.3842 | 15687 | I.S. | 0.38419 |
| 15688 | I.S. | 0.38419 | 15689 | I.S. | 0.38417 | 15690 | I.S. | 0.38414 |
| 15691 | I.S. | 0.38413 | 15692 | I.S. | 0.3841 | 15693 | I.S. | 0.38408 |
| 15694 | I.S. | 0.38407 | 15695 | PMS1 | 0.38407 | 15696 | I.S. | 0.38402 |
| 15697 | I.S. | 0.38402 | 15698 | I.S. | 0.38402 | 15699 | I.S. | 0.38402 |
| 15700 | TGFBR2 | 0.38401 | 15701 | I.S. | 0.38401 | 15702 | I.S. | 0.38399 |
| 15703 | SF3B1 | 0.38398 | 15704 | GALNT12 | 0.38397 | 15705 | FANCB | 0.38396 |
| 15706 | I.S. | 0.38392 | 15707 | GATA6 | 0.38389 | 15708 | I.S. | 0.38389 |
| 15709 | GRIN2A | 0.38385 | 15710 | I.S. | 0.38384 | 15711 | I.S. | 0.38382 |
| 15712 | FANCD2 | 0.38381 | 15713 | I.S. | 0.38378 | 15714 | I.S. | 0.38375 |
| 15715 | I.S. | 0.38374 | 15716 | I.S. | 0.38372 | 15717 | SMARCA4 | 0.38372 |
| 15718 | I.S. | 0.38372 | 15719 | I.S. | 0.3837 | 15720 | PRKDC | 0.3837 |
| 15721 | I.S. | 0.38369 | 15722 | I.S. | 0.38369 | 15723 | I.S. | 0.38369 |
| 15724 | I.S. | 0.38369 | 15725 | I.S. | 0.38365 | 15726 | PDGFRA | 0.38364 |
| 15727 | I.S. | 0.38364 | 15728 | I.S. | 0.38363 | 15729 | I.S. | 0.3836 |
| 15730 | PDK1 | 0.38358 | 15731 | I.S. | 0.38357 | 15732 | I.S. | 0.38348 |
| 15733 | I.S. | 0.38348 | 15734 | RB1 | 0.38348 | 15735 | I.S. | 0.38348 |
| 15736 | I.S. | 0.38348 | 15737 | NPM1 | 0.38345 | 15738 | I.S. | 0.38344 |
| 15739 | MVK | 0.38343 | 15740 | EPAS1 | 0.38342 | 15741 | I.S. | 0.38342 |
| 15742 | I.S. | 0.3834 | 15743 | I.S. | 0.38336 | 15744 | I.S. | 0.38335 |
| 15745 | I.S. | 0.38335 | 15746 | PHF6 | 0.38334 | 15747 | I.S. | 0.38333 |
| 15748 | I.S. | 0.38332 | 15749 | I.S. | 0.3833 | 15750 | I.S. | 0.38329 |
| 15751 | I.S. | 0.38328 | 15752 | I.S. | 0.38328 | 15753 | I.S. | 0.38328 |
| 15754 | I.S. | 0.38328 | 15755 | I.S. | 0.38327 | 15756 | I.S. | 0.38327 |
| 15757 | I.S. | 0.38327 | 15758 | I.S. | 0.38324 | 15759 | I.S. | 0.38324 |
| 15760 | I.S. | 0.38323 | 15761 | I.S. | 0.38323 | 15762 | I.S. | 0.38321 |
| 15763 | I.S. | 0.38315 | 15764 | I.S. | 0.38315 | 15765 | I.S. | 0.38312 |
| 15766 | I.S. | 0.38312 | 15767 | I.S. | 0.38312 | 15768 | I.S. | 0.38312 |
| 15769 | TERF1 | 0.38312 | 15770 | NUP93 | 0.38312 | 15771 | I.S. | 0.3831 |
| 15772 | I.S. | 0.3831 | 15773 | EPAS1 | 0.3831 | 15774 | CTNNA1 | 0.38309 |
| 15775 | I.S. | 0.38307 | 15776 | I.S. | 0.38304 | 15777 | NBN | 0.38304 |
| 15778 | AKT3 | 0.38304 | 15779 | I.S. | 0.38303 | 15780 | I.S. | 0.38301 |
| 15781 | I.S. | 0.383 | 15782 | I.S. | 0.383 | 15783 | I.S. | 0.383 |
| 15784 | I.S. | 0.38297 | 15785 | I.S. | 0.38295 | 15786 | EMSY | 0.38295 |
| 15787 | I.S. | 0.38295 | 15788 | ABL1 | 0.38295 | 15789 | NLRP3 | 0.38295 |
| 15790 | I.S. | 0.38294 | 15791 | I.S. | 0.38294 | 15792 | I.S. | 0.38292 |
| 15793 | I.S. | 0.38291 | 15794 | I.S. | 0.38289 | 15795 | I.S. | 0.38287 |
| 15796 | I.S. | 0.38286 | 15797 | I.S. | 0.38285 | 15798 | I.S. | 0.38282 |
| 15799 | I.S. | 0.38282 | 15800 | I.S. | 0.38282 | 15801 | FUBP1 | 0.38279 |
| 15802 | BCOR | 0.38277 | 15803 | I.S. | 0.38276 | 15804 | I.S. | 0.38276 |
| 15805 | I.S. | 0.38274 | 15806 | I.S. | 0.38273 | 15807 | I.S. | 0.38271 |
| 15808 | I.S. | 0.38271 | 15809 | I.S. | 0.38271 | 15810 | I.S. | 0.38271 |
| 15811 | I.S. | 0.38271 | 15812 | EP300 | 0.38271 | 15813 | I.S. | 0.38269 |
| 15814 | I.S. | 0.38269 | 15815 | I.S. | 0.38268 | 15816 | I.S. | 0.38268 |
| 15817 | I.S. | 0.38267 | 15818 | I.S. | 0.38265 | 15819 | I.S. | 0.38265 |
| 15820 | I.S. | 0.38265 | 15821 | I.S. | 0.38265 | 15822 | I.S. | 0.38265 |
| 15823 | I.S. | 0.38265 | 15824 | I.S. | 0.38265 | 15825 | I.S. | 0.38264 |
| 15826 | I.S. | 0.38262 | 15827 | I.S. | 0.38259 | 15828 | I.S. | 0.38259 |
| 15829 | I.S. | 0.38259 | 15830 | I.S. | 0.38257 | 15831 | SAMD9L | 0.38257 |
| 15832 | SETD2 | 0.38256 | 15833 | I.S. | 0.38255 | 15834 | I.S. | 0.38253 |
| 15835 | RBBP6 | 0.38252 | 15836 | I.S. | 0.38251 | 15837 | RAF1 | 0.38251 |
| 15838 | I.S. | 0.38251 | 15839 | I.S. | 0.38251 | 15840 | I.S. | 0.3825 |
| 15841 | I.S. | 0.3825 | 15842 | I.S. | 0.38248 | 15843 | I.S. | 0.38247 |
| 15844 | I.S. | 0.38245 | 15845 | LRP1B | 0.38245 | 15846 | I.S. | 0.38244 |
| 15847 | I.S. | 0.38242 | 15848 | I.S. | 0.38241 | 15849 | I.S. | 0.38238 |
| 15850 | I.S. | 0.38238 | 15851 | I.S. | 0.38238 | 15852 | I.S. | 0.38238 |
| 15853 | I.S. | 0.38236 | 15854 | I.S. | 0.38236 | 15855 | I.S. | 0.38235 |
| 15856 | I.S. | 0.38232 | 15857 | I.S. | 0.3823 | 15858 | I.S. | 0.38229 |
| 15859 | NFKBIA | 0.38229 | 15860 | I.S. | 0.38227 | 15861 | CTNNA1 | 0.38226 |
| 15862 | I.S. | 0.38224 | 15863 | EPHA7 | 0.38224 | 15864 | I.S. | 0.38224 |
| 15865 | I.S. | 0.38223 | 15866 | ATRX | 0.38221 | 15867 | I.S. | 0.3822 |
| 15868 | I.S. | 0.38218 | 15869 | RAF1 | 0.38218 | 15870 | I.S. | 0.38215 |
| 15871 | I.S. | 0.38214 | 15872 | I.S. | 0.38212 | 15873 | GRIN2A | 0.38211 |
| 15874 | STAT3 | 0.38209 | 15875 | I.S. | 0.38208 | 15876 | I.S. | 0.38205 |
| 15877 | I.S. | 0.38202 | 15878 | I.S. | 0.38202 | 15879 | I.S. | 0.38202 |
| 15880 | I.S. | 0.382 | 15881 | I.S. | 0.382 | 15882 | PBRM1 | 0.38197 |
| 15883 | I.S. | 0.38197 | 15884 | I.S. | 0.38196 | 15885 | SMC1A | 0.38195 |
| 15886 | I.S. | 0.38194 | 15887 | I.S. | 0.38194 | 15888 | I.S. | 0.38191 |
| 15889 | I.S. | 0.38191 | 15890 | I.S. | 0.38191 | 15891 | I.S. | 0.38188 |
| 15892 | I.S. | 0.38188 | 15893 | SMAD2 | 0.38187 | 15894 | PREX2 | 0.38185 |
| 15895 | I.S. | 0.38184 | 15896 | I.S. | 0.38184 | 15897 | I.S. | 0.38184 |
| 15898 | I.S. | 0.3818 | 15899 | ARID2 | 0.3818 | 15900 | I.S. | 0.38179 |
| 15901 | I.S. | 0.38179 | 15902 | I.S. | 0.38179 | 15903 | I.S. | 0.38176 |
| 15904 | I.S. | 0.38175 | 15905 | RB1 | 0.38175 | 15906 | I.S. | 0.38172 |
| 15907 | I.S. | 0.38171 | 15908 | ATM | 0.3817 | 15909 | I.S. | 0.38169 |
| 15910 | PRKDC | 0.38167 | 15911 | I.S. | 0.38167 | 15912 | I.S. | 0.38167 |
| 15913 | I.S. | 0.38167 | 15914 | I.S. | 0.38167 | 15915 | I.S. | 0.38166 |
| 15916 | I.S. | 0.38164 | 15917 | I.S. | 0.38163 | 15918 | I.S. | 0.38161 |
| 15919 | I.S. | 0.38159 | 15920 | STAG2 | 0.38159 | 15921 | I.S. | 0.38155 |
| 15922 | I.S. | 0.38155 | 15923 | I.S. | 0.38155 | 15924 | I.S. | 0.3815 |
| 15925 | I.S. | 0.38149 | 15926 | I.S. | 0.38149 | 15927 | I.S. | 0.38147 |
| 15928 | CUX1 | 0.38147 | 15929 | I.S. | 0.38146 | 15930 | I.S. | 0.38146 |
| 15931 | ATRX | 0.38146 | 15932 | I.S. | 0.38144 | 15933 | ERG | 0.38144 |
| 15934 | I.S. | 0.38144 | 15935 | EPHA7 | 0.38142 | 15936 | I.S. | 0.3814 |
| 15937 | I.S. | 0.38138 | 15938 | I.S. | 0.38138 | 15939 | FLT4 | 0.38137 |
| 15940 | I.S. | 0.38134 | 15941 | I.S. | 0.38134 | 15942 | I.S. | 0.38134 |
| 15943 | I.S. | 0.38134 | 15944 | IGF2 | 0.38134 | 15945 | EPHA5 | 0.38131 |
| 15946 | I.S. | 0.38131 | 15947 | I.S. | 0.3813 | 15948 | I.S. | 0.38129 |
| 15949 | I.S. | 0.38128 | 15950 | I.S. | 0.38128 | 15951 | I.S. | 0.38126 |
| 15952 | I.S. | 0.38126 | 15953 | I.S. | 0.38126 | 15954 | BTK | 0.38125 |
| 15955 | I.S. | 0.38125 | 15956 | I.S. | 0.38123 | 15957 | I.S. | 0.38123 |
| 15958 | I.S. | 0.38123 | 15959 | I.S. | 0.38123 | 15960 | I.S. | 0.38122 |
| 15961 | I.S. | 0.38118 | 15962 | I.S. | 0.38115 | 15963 | I.S. | 0.38114 |
| 15964 | I.S. | 0.38114 | 15965 | I.S. | 0.38113 | 15966 | I.S. | 0.38112 |
| 15967 | MPL | 0.38111 | 15968 | CHD2 | 0.38107 | 15969 | I.S. | 0.38106 |
| 15970 | I.S. | 0.38106 | 15971 | I.S. | 0.38104 | 15972 | I.S. | 0.38104 |
| 15973 | I.S. | 0.38104 | 15974 | I.S. | 0.38102 | 15975 | I.S. | 0.38101 |
| 15976 | PRKDC | 0.38101 | 15977 | EP300 | 0.38101 | 15978 | I.S. | 0.38099 |
| 15979 | I.S. | 0.38097 | 15980 | I.S. | 0.38096 | 15981 | I.S. | 0.38096 |
| 15982 | I.S. | 0.38095 | 15983 | I.S. | 0.38095 | 15984 | I.S. | 0.38094 |
| 15985 | I.S. | 0.38093 | 15986 | MRE11 | 0.38093 | 15987 | I.S. | 0.38093 |
| 15988 | I.S. | 0.38092 | 15989 | SMO | 0.3809 | 15990 | TSC1 | 0.38087 |
| 15991 | I.S. | 0.38085 | 15992 | I.S. | 0.38084 | 15993 | I.S. | 0.38083 |
| 15994 | I.S. | 0.38081 | 15995 | I.S. | 0.38081 | 15996 | I.S. | 0.38081 |
| 15997 | I.S. | 0.3808 | 15998 | MED12 | 0.38078 | 15999 | I.S. | 0.38078 |
| 16000 | I.S. | 0.38075 | 16001 | I.S. | 0.38074 | 16002 | I.S. | 0.38074 |
| 16003 | MTOR | 0.38073 | 16004 | I.S. | 0.38071 | 16005 | I.S. | 0.38069 |
| 16006 | I.S. | 0.38069 | 16007 | I.S. | 0.38068 | 16008 | ABCB7 | 0.38063 |
| 16009 | I.S. | 0.38062 | 16010 | I.S. | 0.3806 | 16011 | I.S. | 0.3806 |
| 16012 | I.S. | 0.3806 | 16013 | I.S. | 0.3806 | 16014 | I.S. | 0.3806 |
| 16015 | I.S. | 0.38058 | 16016 | SPEN | 0.38057 | 16017 | I.S. | 0.38057 |
| 16018 | I.S. | 0.38057 | 16019 | I.S. | 0.38057 | 16020 | I.S. | 0.38057 |
| 16021 | SPEN | 0.38054 | 16022 | I.S. | 0.38052 | 16023 | I.S. | 0.38051 |
| 16024 | I.S. | 0.38051 | 16025 | I.S. | 0.38049 | 16026 | MITF | 0.38049 |
| 16027 | I.S. | 0.38049 | 16028 | I.S. | 0.38048 | 16029 | I.S. | 0.38046 |
| 16030 | NOTCH3 | 0.38045 | 16031 | I.S. | 0.38044 | 16032 | I.S. | 0.38043 |
| 16033 | I.S. | 0.38042 | 16034 | I.S. | 0.38042 | 16035 | ARID2 | 0.3804 |
| 16036 | I.S. | 0.38039 | 16037 | I.S. | 0.38039 | 16038 | I.S. | 0.38037 |
| 16039 | I.S. | 0.38036 | 16040 | I.S. | 0.38034 | 16041 | I.S. | 0.38033 |
| 16042 | I.S. | 0.3803 | 16043 | RICTOR | 0.3803 | 16044 | DOT1L | 0.3803 |
| 16045 | I.S. | 0.38027 | 16046 | I.S. | 0.38027 | 16047 | I.S. | 0.38027 |
| 16048 | I.S. | 0.38024 | 16049 | NSD2 | 0.38024 | 16050 | I.S. | 0.38024 |
| 16051 | I.S. | 0.38024 | 16052 | I.S. | 0.38022 | 16053 | FLT4 | 0.38021 |
| 16054 | I.S. | 0.38019 | 16055 | I.S. | 0.38019 | 16056 | FANCG | 0.38015 |
| 16057 | I.S. | 0.38014 | 16058 | I.S. | 0.38013 | 16059 | I.S. | 0.38013 |
| 16060 | I.S. | 0.3801 | 16061 | I.S. | 0.38009 | 16062 | I.S. | 0.38009 |
| 16063 | I.S. | 0.38007 | 16064 | I.S. | 0.38007 | 16065 | I.S. | 0.38006 |
| 16066 | TCF3 | 0.38006 | 16067 | I.S. | 0.38 | 16068 | I.S. | 0.38 |
| 16069 | SPOP | 0.38 | 16070 | I.S. | 0.38 | 16071 | I.S. | 0.37998 |
| 16072 | I.S. | 0.37998 | 16073 | ANKRD26 | 0.37998 | 16074 | I.S. | 0.37998 |
| 16075 | I.S. | 0.37998 | 16076 | I.S. | 0.37996 | 16077 | TNFAIP3 | 0.37995 |
| 16078 | I.S. | 0.37994 | 16079 | I.S. | 0.37994 | 16080 | I.S. | 0.37992 |
| 16081 | I.S. | 0.37992 | 16082 | I.S. | 0.37991 | 16083 | I.S. | 0.37988 |
| 16084 | I.S. | 0.37987 | 16085 | I.S. | 0.37987 | 16086 | I.S. | 0.37986 |
| 16087 | TERF2IP | 0.37985 | 16088 | I.S. | 0.37983 | 16089 | I.S. | 0.37982 |
| 16090 | I.S. | 0.37982 | 16091 | KDM6A | 0.37978 | 16092 | I.S. | 0.37977 |
| 16093 | I.S. | 0.37976 | 16094 | I.S. | 0.37974 | 16095 | I.S. | 0.37974 |
| 16096 | I.S. | 0.37973 | 16097 | CHD2 | 0.37973 | 16098 | BRAF | 0.37973 |
| 16099 | I.S. | 0.37971 | 16100 | I.S. | 0.37971 | 16101 | I.S. | 0.37971 |
| 16102 | I.S. | 0.37971 | 16103 | I.S. | 0.37967 | 16104 | I.S. | 0.37966 |
| 16105 | I.S. | 0.37965 | 16106 | I.S. | 0.37965 | 16107 | I.S. | 0.37965 |
| 16108 | I.S. | 0.37964 | 16109 | RAD54L | 0.37963 | 16110 | I.S. | 0.37963 |
| 16111 | I.S. | 0.37963 | 16112 | I.S. | 0.37963 | 16113 | I.S. | 0.37962 |
| 16114 | I.S. | 0.37961 | 16115 | I.S. | 0.37961 | 16116 | I.S. | 0.37959 |
| 16117 | I.S. | 0.37959 | 16118 | I.S. | 0.37959 | 16119 | I.S. | 0.37958 |
| 16120 | I.S. | 0.37957 | 16121 | KIF23 | 0.37957 | 16122 | I.S. | 0.37956 |
| 16123 | ATRX | 0.37956 | 16124 | I.S. | 0.37956 | 16125 | I.S. | 0.37953 |
| 16126 | I.S. | 0.37953 | 16127 | I.S. | 0.37953 | 16128 | I.S. | 0.3795 |
| 16129 | I.S. | 0.37947 | 16130 | MDM2 | 0.37946 | 16131 | I.S. | 0.37946 |
| 16132 | I.S. | 0.37945 | 16133 | I.S. | 0.37945 | 16134 | I.S. | 0.37945 |
| 16135 | I.S. | 0.37945 | 16136 | SPEN | 0.37944 | 16137 | I.S. | 0.37944 |
| 16138 | I.S. | 0.37944 | 16139 | I.S. | 0.37943 | 16140 | SETD2 | 0.37942 |
| 16141 | I.S. | 0.37942 | 16142 | I.S. | 0.37942 | 16143 | I.S. | 0.3794 |
| 16144 | GNAS | 0.37939 | 16145 | I.S. | 0.37939 | 16146 | I.S. | 0.37938 |
| 16147 | I.S. | 0.37938 | 16148 | I.S. | 0.37937 | 16149 | LRP1B | 0.37936 |
| 16150 | I.S. | 0.37935 | 16151 | RANBP2 | 0.37935 | 16152 | ARID2 | 0.3793 |
| 16153 | CSF3R | 0.3793 | 16154 | I.S. | 0.37929 | 16155 | I.S. | 0.37925 |
| 16156 | I.S. | 0.37923 | 16157 | I.S. | 0.3792 | 16158 | I.S. | 0.3792 |
| 16159 | I.S. | 0.3792 | 16160 | KAT6A | 0.3792 | 16161 | I.S. | 0.3792 |
| 16162 | I.S. | 0.37917 | 16163 | I.S. | 0.37917 | 16164 | I.S. | 0.37917 |
| 16165 | I.S. | 0.37915 | 16166 | I.S. | 0.37915 | 16167 | I.S. | 0.37914 |
| 16168 | IRS2 | 0.37914 | 16169 | I.S. | 0.37914 | 16170 | I.S. | 0.37912 |
| 16171 | I.S. | 0.37912 | 16172 | I.S. | 0.37912 | 16173 | NF1 | 0.37912 |
| 16174 | EPCAM | 0.37912 | 16175 | I.S. | 0.37911 | 16176 | CBLC | 0.37911 |
| 16177 | I.S. | 0.37909 | 16178 | I.S. | 0.37908 | 16179 | I.S. | 0.37906 |
| 16180 | BCOR | 0.37906 | 16181 | I.S. | 0.37905 | 16182 | I.S. | 0.37905 |
| 16183 | I.S. | 0.37903 | 16184 | SDCCAG8, | 0.37903 | 16185 | I.S. | 0.37902 |
| | | | | AKT3 | | | | |
| 16186 | KIF23 | 0.37902 | 16187 | I.S. | 0.37901 | 16188 | I.S. | 0.37901 |
| 16189 | I.S. | 0.379 | 16190 | DNM2 | 0.37899 | 16191 | I.S. | 0.37899 |
| 16192 | KMT2D | 0.37899 | 16193 | I.S. | 0.37896 | 16194 | I.S. | 0.37896 |
| 16195 | CUX1 | 0.37893 | 16196 | I.S. | 0.37892 | 16197 | I.S. | 0.37891 |
| 16198 | I.S. | 0.37891 | 16199 | I.S. | 0.3789 | 16200 | I.S. | 0.37888 |
| 16201 | I.S. | 0.37888 | 16202 | I.S. | 0.37888 | 16203 | I.S. | 0.37888 |
| 16204 | I.S. | 0.37888 | 16205 | IDH2 | 0.37885 | 16206 | I.S. | 0.37884 |
| 16207 | I.S. | 0.37884 | 16208 | I.S. | 0.37882 | 16209 | I.S. | 0.37882 |
| 16210 | I.S. | 0.37881 | 16211 | I.S. | 0.37881 | 16212 | I.S. | 0.37879 |
| 16213 | I.S. | 0.37879 | 16214 | SPEN | 0.37879 | 16215 | I.S. | 0.37877 |
| 16216 | I.S. | 0.37876 | 16217 | I.S. | 0.37876 | 16218 | STK11 | 0.37873 |
| 16219 | I.S. | 0.37873 | 16220 | I.S. | 0.37871 | 16221 | I.S. | 0.3787 |
| 16222 | I.S. | 0.37868 | 16223 | I.S. | 0.37867 | 16224 | I.S. | 0.37867 |
| 16225 | I.S. | 0.37867 | 16226 | I.S. | 0.37865 | 16227 | I.S. | 0.37864 |
| 16228 | I.S. | 0.37864 | 16229 | I.S. | 0.37863 | 16230 | I.S. | 0.37863 |
| 16231 | STAT6 | 0.37863 | 16232 | I.S. | 0.37859 | 16233 | BRAF | 0.37858 |
| 16234 | I.S. | 0.37858 | 16235 | I.S. | 0.37858 | 16236 | I.S. | 0.37858 |
| 16237 | I.S. | 0.37858 | 16238 | I.S. | 0.37858 | 16239 | I.S. | 0.37855 |
| 16240 | I.S. | 0.37854 | 16241 | I.S. | 0.37854 | 16242 | FANCA | 0.37852 |
| | | | | | | | ZNF276, | |
| 16243 | I.S. | 0.37852 | 16244 | I.S. | 0.37851 | 16245 | I.S. | 0.37847 |
| 16246 | I.S. | 0.37846 | 16247 | I.S. | 0.37843 | 16248 | I.S. | 0.37843 |
| 16249 | FOXP1 | 0.37843 | 16250 | I.S. | 0.37842 | 16251 | I.S. | 0.37839 |
| 16252 | I.S. | 0.37838 | 16253 | I.S. | 0.37838 | 16254 | I.S. | 0.37838 |
| 16255 | I.S. | 0.37837 | 16256 | BCYRN1, | 0.37837 | 16257 | I.S. | 0.37836 |
| | | | | TAF1 | | | | |
| 16258 | I.S. | 0.37835 | 16259 | I.S. | 0.37835 | 16260 | I.S. | 0.37833 |
| 16261 | I.S. | 0.37833 | 16262 | I.S. | 0.37832 | 16263 | I.S. | 0.37829 |
| 16264 | I.S. | 0.37828 | 16265 | I.S. | 0.37828 | 16266 | I.S. | 0.37825 |
| 16267 | I.S. | 0.37825 | 16268 | I.S. | 0.37823 | 16269 | I.S. | 0.37823 |
| 16270 | I.S. | 0.37822 | 16271 | I.S. | 0.37822 | 16272 | I.S. | 0.37819 |
| 16273 | I.S. | 0.37817 | 16274 | GRIN2A | 0.37817 | 16275 | I.S. | 0.37817 |
| 16276 | I.S. | 0.37816 | 16277 | I.S. | 0.37814 | 16278 | I.S. | 0.37813 |
| 16279 | I.S. | 0.37813 | 16280 | I.S. | 0.37811 | 16281 | EPHA5 | 0.3781 |
| 16282 | I.S. | 0.37809 | 16283 | I.S. | 0.37808 | 16284 | I.S. | 0.37807 |
| 16285 | I.S. | 0.37806 | 16286 | ROS1 | 0.37805 | 16287 | I.S. | 0.37805 |
| 16288 | I.S. | 0.37804 | 16289 | I.S. | 0.37804 | 16290 | I.S. | 0.37802 |
| 16291 | I.S. | 0.37802 | 16292 | I.S. | 0.37802 | 16293 | DICER1 | 0.37802 |
| 16294 | I.S. | 0.37801 | 16295 | INPP4B | 0.37799 | 16296 | I.S. | 0.37796 |
| 16297 | RHEB | 0.37793 | 16298 | I.S. | 0.37793 | 16299 | I.S. | 0.37793 |
| 16300 | I.S. | 0.37792 | 16301 | I.S. | 0.37792 | 16302 | EMSY | 0.3779 |
| 16303 | I.S. | 0.3779 | 16304 | I.S. | 0.37789 | 16305 | XPO1 | 0.37786 |
| 16306 | I.S. | 0.37785 | 16307 | I.S. | 0.37781 | 16308 | I.S. | 0.37781 |
| 16309 | I.S. | 0.37781 | 16310 | I.S. | 0.37778 | 16311 | I.S. | 0.37776 |
| 16312 | I.S. | 0.37776 | 16313 | SUFU | 0.37776 | 16314 | I.S. | 0.37776 |
| 16315 | I.S. | 0.37774 | 16316 | KMT2A | 0.37772 | 16317 | I.S. | 0.37772 |
| 16318 | DDX41 | 0.37772 | 16319 | I.S. | 0.37772 | 16320 | I.S. | 0.37772 |
| 16321 | I.S. | 0.37772 | 16322 | I.S. | 0.37769 | 16323 | I.S. | 0.37769 |
| 16324 | EPHB1 | 0.37763 | 16325 | I.S. | 0.37763 | 16326 | MED12 | 0.37763 |
| 16327 | I.S. | 0.37762 | 16328 | I.S. | 0.37762 | 16329 | I.S. | 0.37762 |
| 16330 | NTRK1 | 0.37761 | 16331 | I.S. | 0.37761 | 16332 | I.S. | 0.3776 |
| 16333 | INPP4B | 0.3776 | 16334 | I.S. | 0.3776 | 16335 | SEC23B | 0.3776 |
| 16336 | I.S. | 0.37757 | 16337 | I.S. | 0.37756 | 16338 | I.S. | 0.37756 |
| 16339 | CBFB | 0.37756 | 16340 | I.S. | 0.37755 | 16341 | RB1 | 0.37755 |
| 16342 | I.S. | 0.37751 | 16343 | I.S. | 0.37748 | 16344 | I.S. | 0.37748 |
| 16345 | I.S. | 0.37746 | 16346 | I.S. | 0.37745 | 16347 | I.S. | 0.37743 |
| 16348 | I.S. | 0.37742 | 16349 | I.S. | 0.37741 | 16350 | I.S. | 0.37741 |
| 16351 | I.S. | 0.3774 | 16352 | I.S. | 0.3774 | 16353 | I.S. | 0.3774 |
| 16354 | I.S. | 0.3774 | 16355 | I.S. | 0.37736 | 16356 | SMARCA4 | 0.37736 |
| 16357 | I.S. | 0.37736 | 16358 | I.S. | 0.37736 | 16359 | I.S. | 0.37734 |
| 16360 | I.S. | 0.37733 | 16361 | I.S. | 0.37732 | 16362 | I.S. | 0.3773 |
| 16363 | EP300 | 0.3773 | 16364 | I.S. | 0.37728 | 16365 | RAD51 | 0.37728 |
| 16366 | I.S. | 0.37727 | 16367 | I.S. | 0.37727 | 16368 | I.S. | 0.37724 |
| 16369 | I.S. | 0.37724 | 16370 | I.S. | 0.37724 | 16371 | I.S. | 0.37724 |
| 16372 | I.S. | 0.37721 | 16373 | I.S. | 0.37721 | 16374 | TERT | 0.37721 |
| 16375 | I.S. | 0.3772 | 16376 | I.S. | 0.37718 | 16377 | I.S. | 0.37718 |
| 16378 | I.S. | 0.37717 | 16379 | I.S. | 0.37715 | 16380 | I.S. | 0.37712 |
| 16381 | I.S. | 0.3771 | 16382 | KDM6A | 0.3771 | 16383 | BCYRN1, | 0.3771 |
| | | | | | | | TAF1 | |

The classification system was trained with a selected subset of the most relevant candidate segments of the genes. The Geometric Mean Naïve Bayesian (GMNB) was applied as the classifier to predict specific cancer. GMNB is a generalized naïve Bayesian classifier that applies a geometric mean to the likelihood product, which eliminate the underflow problem commonly associated with the standard naïve Bayesian classifiers with high dimensionality. The effectiveness of the trained GMNB classifier for the selected subset of the most relevant candidate segments, referred to herein as the current relevant subset of segments, was determined. The selected subset of the most relevant candidate segments was modified by removing the least relevant candidate segment forming a new current relevant subset of candidate segments. A GMNB classifier was trained on the new current relevant subset resulting in a new current trained GMNB classifier whose effectiveness was determined. The effectiveness of the new current GMNB classifier was compared to the immediately prior GMNB classifier. The comparison determined whether to iteratively continue modifying the selected subset of most relevant candidate segments, training a new GMNB classifier, evaluating the effectiveness of the new current GMNB classifier, and comparing it to that of the immediately prior GMNB classifier, or to identify the immediately prior subset of the most relevant candidate segments as the set of target segments relevant to the specific cancer of interest.

The iterative process for selection of target segments is described above with respect to FIGS. 2A and 2B. This iterative process results in identification of which segments in the set of target genes are most useful for effective classification of a sample as having a BRCA1/2 mutation or HRD without overfitting and a classifier trained with the most useful segments.

Once the set of target segments relevant to the specific cancer of interest is identified, a new GMNB classifier can be trained using training data for the set of target segments to generate the trained GMNB classifier for classification of new samples. In some embodiments, the training data for training the new GMNB classifier for classification of new samples can include all of the training data that was used to determine the set of target segments. In some embodiments, the training data for training the new GMNB classifier for classification of new samples can include at least some of the training data used to determine the target segments. In some embodiments, the training data for training the new GMNB classifier for classification of new samples can include at least some of the training data used to determine the target segments and at least some new training data. In some embodiments, the training data for training the new GMNB classifier for classification of new samples can include new training data.

Results:

High Sensitivity and Specificity in Predicting the Presence of BRCA] 2 Mutations

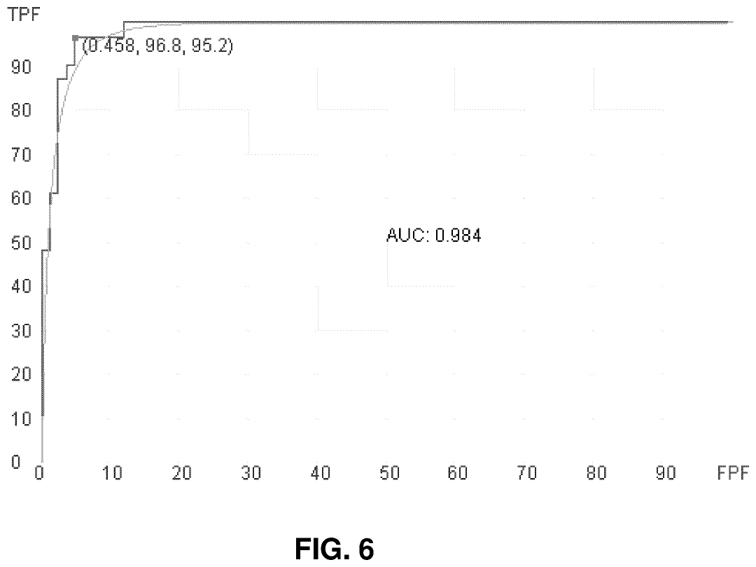

Using log 2 normalized copy number of the 26,940 segments (bins) of the 434 sequenced genes in the machine learning classifier, the inventors demonstrated the ability to distinguish between BRCA1/2 mutated samples and BRCA1/2 unmutated samples. The samples employed to train the classifier included 31 samples with confirmed BRCA1/2 mutation and 84 samples confirmed negative for mutations in BRCA1/2 or any of the genes implicated in DSB repair. The automated machine learning system developed by the inventors selected 16,383 segments (e.g., markers) (bins), also referred to herein as target segments, for best separation between BRCA1/2 positive and negative samples. The receiver operating characteristic curve showed AUC (area under the curve) of 0.984 (FIG. 6). Using smaller numbers of segments (e.g., markers) showed significantly less prediction, while using a larger number of segments (e.g., markers) did not change the prediction significantly. Based on using a cut-off point of 0.486, the sensitivity was 90% and specificity was 98% (Table 4). High specificity was selected over sensitivity to improve prediction of response to therapy. The actual log 2 copy ratio by CNVkit of one positive and one negative example is shown in FIG. 7.

TABLE 4
Sensitivity and specificity of detecting the presence
of HRD associated with BRCA1/2 using copy number
variation (CNV) and machine learning algorithm.

| | | 95% Confidence Interval |
|---|---|---|
| Sensitivity | 90% | 73%-97.5% |
| Specificity | 98% | 90%-100% |
| PPV | 93% | 76.5%-99% |
| NPV | 96% | 89%-99% |

PPV, positive predictive value; NPV, negative predictive value.

Predicting HRD in Cancers with Mutations in Various DSB Repair Genes as Compared with DSB-Null Cancers

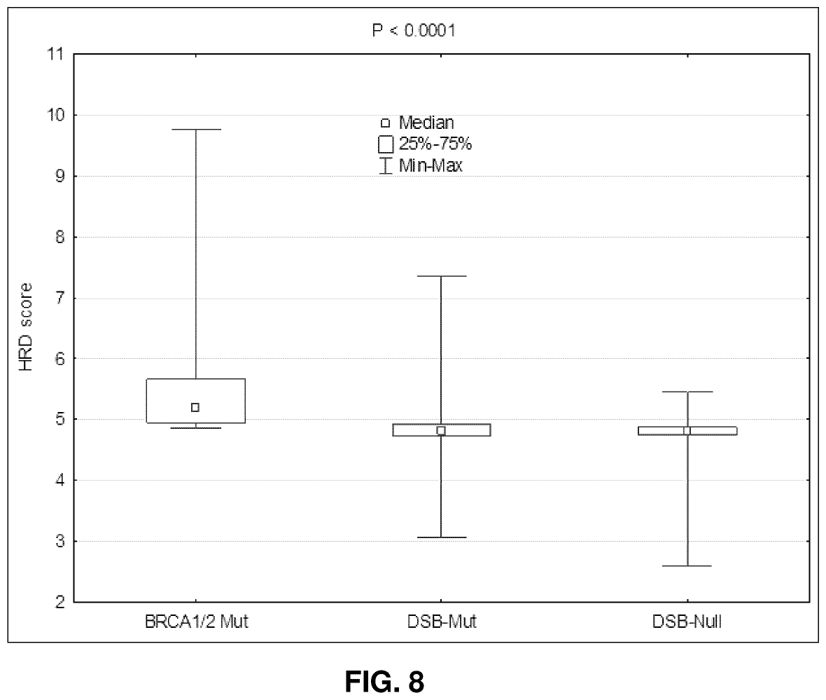

To measure the value of the developed machine learning classifier after training to predict BRCA1/2 positive tumors, samples from 213 cancers without mutations in any of the DSB genes and 114 cancers with mutations in one of the genes involved in DSB repair were tested. These DSB genes-positive samples included cancers with mutations in ATM (a gene consisting of 66 exons that encode a 350 kDa protein kinase enzyme; see Moslemi, M. et al. BMC Cancer (2021) 21: article 27) (N=36), ATR (ATR serine/threonine kinase, a gene that encodes a protein that is a member of the P13/P14-kinase family that functions in the phosphorylation of checkpoint kinase CHK1, RAD17, RAD9 and tumor suppressor protein BRCA1; see Kamitz, L M and Zou, L. Clin. Cancer Res. (2015) 21 (21): 4780-85) (N=17), CDK12 (cyclin dependent kinase 12) (N=14), Fanconi anemia genes (N=16), NBN (provides instructions for making a protein called nibrin, which participates in DNA repair) (N=12), RAD50 (a gene that encodes a protein that functions in double-strand break repair) (N=9), RAD50 L (a highly conserved DNA double-strand break repair gene; see Fan, C. et al. Intl J. Cancer (2018) 143 (8): 1935-42)(N=1), RAD51 (a homolog of bacterial RecA protein, which interacts with BRCA1 and BRCA2 for homologous recombination repair; see Sekhar, D. et al. Scientific Reports (2015) 5: article 11588) (N=6), and RAD54 L (a gene involved in homologous recombination) (N=4). The trained classifier classified 44 of the 114 samples with mutations in one of the genes involved in DSB repair (39%) as having structural genomic abnormalities similar to those detected in BRCA1/2-positive cases, indicating high positivity for HRD. These HRD-positive cases had mutations in ATM (N=13), CDK12 (N=5), Fanconi genes (6), ATR (N=26), NBN (N=6), RAD51 (N=3), RAD54 L (N=1) and RAD50 (N=4). Testing 213 random cancer samples without DSB mutations showed 68 cancer (32%) positive scores similar to those seen in BRCA1/2 samples (FIG. 8). There was a significant difference in score between DSB-positive other than BRCA1/2 tumors and BRCA1/2-positive cases (P<0.0001), but there was no difference between DSB-positive cases and DSB-null cancers (P=0.47). This suggests that cancers with mutations in DSB genes other than BRCA1/2 are heterogeneous and overall more similar to DSB-null cases than to BRCA1/2-mutant cancers.

Using 31 cancers with BRCA1/2 mutations and 84 tumors without mutations in any of the DSB repair genes (DSB-null), the machine learning algorithm demonstrated high sensitivity (90%, 95% CI: 73%-97.5%) and specificity (98%, 95% CI:90%-100%) in distinguishing between these two groups of tumors. Testing of 114 tumors with mutations in DSB repair genes other than BRCA1/2 showed 39% positivity for HRD similar to that seen in BRCA1/2. Testing 213 additional DSB-null cancers showed HRD positivity similar to BRCA1/2 in 32% of cases.

### DISCUSSION

Clinical studies have suggested that clinical response to DSB-inducing agents is best in cancers that have genomic abnormalities dictated by BRCA1/2 mutations. These genomic abnormalities are typically manifested by structural chromosomal abnormalities resulting from homologous recombination deficiency including copy number variations, translocations, and LOH. Tumors with BRCA1/2 are considered the gold standard for these abnormalities.

Tumors with abnormalities similar to those seen in cases with BRCA1/2 are currently classified as eligible for treatment with DSB-inducer agents. The chromosomal structural abnormalities historically were measured using a complicated approach involving evaluation of LOH, TAI, and LST. The results of these measurements are combined giving a score that is used to determine which tumor might respond to DSB-inducer therapy. Multiple studies demonstrated that this approach might be useful but not robust enough to accurately select patients.

With the advances in NGS, chromosomal structural abnormalities can be measured and quantified. Kim, S J, et al. measured HRD using the same approach (LOH, TAI, and LST) using NGS and whole exome sequencing and demonstrated that HRD-high tumors had significantly (P=0.003) higher pathologic complete response (pCR) rates and higher near-pCR rates (P=0.049) compared with those of the HRD-low tumors. [See Determining homologous recombination deficiency scores with whole exome sequencing and their association with responses to neoadjuvant chemotherapy in breast cancer. Translational Oncology. 2021 February; 14(2): 100986]. High score was detected in tumors with germline mutations in DSB-related genes, but not in somatic mutations. Eeckhoutt, A, et al. used a shallow whole genome sequencing for measuring LGA and predicting HRD. [See ShallowHRD: detection of homologous recombination deficiency from shallow whole genome sequencing. Bioinformatics. 2020 June 15; 36(12):3888-3889]. Whole genome sequencing and mutation profile was also used by Davies, H, et al. for detecting HRD using a supervised lasso logistic regression model. [See HRDetect is a predictor of BRCA1 and BRCA2 deficiency based on mutational signatures. Nat Med. 2017 April; 23(4):517-525].

This Example presents data for predicting or classifying a sample having a BRCA1/2 mutation or a level of HRD similar to that caused by a BRCA1/2 mutation based on using routinely generated sequencing data from targeted molecular profiling used for the detection of various clinically relevant mutations in solid tumors, measuring tumor mutation burden and microsatellite instability (MSI). The assay is specifically designed to be relatively simple, cost-effective and amenable for adaptation in routine clinical laboratories. The panel included targeted coding regions of 434 genes. The normalized log 2 ratio of sequenced fragments (bins) generated by CNVkit software was used in a machine learning approach to develop a model for classifying tumors with BRCA1/2 mutations vs. tumors without BRCA1/2 mutations. Practically, this method quantified gains and losses of various DNA fragments in the genomic areas covered by these 434 genes, then used a machine learning approach for classifying cases. As shown in FIG. 6, the method distinguished between BRCA1/2-positive and BRCA1/2-negative cases with high sensitivity and specificity (AUC=98.4%). When this method was used in testing samples with mutations in one of the other DSB genes (other than BRCA1/2) and cancers without mutations in any of the DSB genes, the trained classifier predicted that 39% of the cases with mutations in one of the DSB genes other than BRCA1/2 can be classified as having genomic structural abnormalities similar to those seen in BRCA1/2-positive cases, while 32% of DSB-null cancers had HRD similar to that seen in the BRCA1/2 cases. There was a significant difference (P<0.0001) between BRCA1/2-positive cancers and both other groups, specifically, the DSB-positive cases (no BRCA1/2 mutations) and DSB-null cases. There was no significant difference (P=0.47) between DSB-positive (no BRCA1/2 mutations) and DSB-null cases (FIG. 8). This suggests that mutations in DSB other than BRCA1/2 are associated with increased tendency to have HRD, but not significantly higher than DSB-null cases. This may explain the reason that clinical trials failed to show significant improvement in outcome in cancers with mutations in DSB genes other than BRCA1/2 when treated with PARPi. [Laden, M. M. et al., Homologous Recombination Deficiency Testing for BRCA-Like Tumors: The Road to Clinical Validation. Cancers (Basel). 2021 Feb. 28; 13(5):1004].

In summary, using this method, cancers can be classified into two groups: (a) High HRD score including BRCA1/2-positive and cases with score similar to BRCA1/2-positive irrespective of if they have mutations in DSB genes or not; (b) Negative HRD score including cancers with HRD score lower than that seen in cases with BRCA1/2 genes. This indicates that high score cancers can be considered eligible for treatment with DSB-inducing drugs. However, the demonstrated high sensitivity and specificity of this approach in predicting BRCA1/2-associated genomic abnormalities indicates that this approach has high potential for predicting response to PARPi. Furthermore, this functional approach can predict increased susceptibility to homologous recombination due to causes other than mutations, such as methylation, deletion in DSB-repair genes, multigenic factors, and others.

### CONCLUSIONS

This data demonstrated that CNV when combined with a machine learning approach can reliably predict the presence of BRCA1/2 level HRD with high specificity. Using BRCA1/2 mutant cases as gold standard, this method can be used to predict HRD in cancers with mutations in other DSB genes as well as in DSB-null tumors. Because best response to DSB-inducing agents is associated with CNV changes similar to those seen in BRCA1/2 mutations, the demonstration of the presence of such changes in a tumor using this method approach implies response to DSB-inducers.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The cited references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

1. A method comprising:
a) accessing or obtaining a sample from a subject who has a cancer;
b) determining sequences of segments and copy number for the sequences of a plurality of target genes in the sample using next generation sequencing;
c) determining copy number variation for each of a plurality of target segments from the determined sequences and copy number for the sequences of the plurality of target genes; and
d) at least one of:
(1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or
(2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or
(3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene by applying the trained classifier and using the copy number variation for the plurality of target segments as input attributes for the trained classifier, and
wherein the cancer is a breast cancer; or an ovarian cancer; or one or more of lymphoma, leukemia, or a solid tumor.

2. The method claim 1, further comprising e) at least one of
(1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or
(2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; or
(3) where the sample is classified as having a mutation of the BRCA1 or the BRCA2 gene or a genomic structural abnormality biologically similar to a mutation of the BRCA1 or the BRCA2 gene indicating a similar level of HRD as that caused by a mutation of the BRCA1 or the BRCA2 gene irrespective of the mutated gene involved, identifying the subject as a candidate for treatment with a double strand break-inducing agent.

3. The method of claim 2, further comprising f) at least one of:
(1) where the sample is classified as not having a high level of HRD and therefore not having the same or similar structural abnormality as a level of HRD associated with a mutation of the BRCA1 or the BRCA2 gene irrespective of the mutated gene involved, identifying the subject as not a candidate for treatment with a double strand break-inducing agent; or
(2) where the sample is classified as not having a mutation of the BRCA1 or the BRCA2 gene or a level of HRD similar to that caused by a mutation of the BRCA1 or the BRCA2 gene irrespective of the mutated gene involved, identifying the subject as not a candidate for treatment with a double strand break-inducing agent; or
(3) where the sample is classified as not having a mutation of the BRCA1 or the BRCA2 gene or a genomic structural abnormality biologically similar to a mutation of the BRCA1 gene or the BRCA2 gene indicating a similar level of HRD as that caused by a mutation of the BRCA1 gene or the BRCA2 gene irrespective of the mutated gene involved, identifying the subject as not a candidate for treatment with a double strand break-inducing agent.

4. (canceled)

5. The method of claim 1, wherein the training data for the trained classifier comprises a first group of samples with confirmed mutations in the BRCA1 gene and/or the BRCA2 gene, and a second group of samples that are confirmed negative for mutations in the BRCA1 gene and the BRCA2 gene and confirmed negative for mutations in any double strand break repair genes; and/or
wherein the training data for the trained classifier further comprises a third group of samples with mutations in one double strand break repair gene.

6. (canceled)

7. The method of claim 1, wherein the trained classifier is a geometric mean naïve Bayesian classifier, or wherein the method further comprises training a classifier to produce the trained classifier.

8. (canceled)

9. The method of claim 1, further comprising determining the plurality of target segments whose copy number variation is used for classification by steps including:
(A) accessing or obtaining training samples including a first group of samples with confirmed mutations in the BRCA1 gene and/or the BRCA2 gene, and a second group of samples that are confirmed negative for mutations in the BRCA1 gene and the BRCA2 gene and confirmed negative for mutations in any double strand break repair genes;
(B) for each training sample,
(i) determining sequences and copy number of a plurality of candidate genes in the training sample using next generation sequencing; and
(ii) determining copy number variation for each of a plurality of candidate segments from the determined sequences and copy number of the plurality of candidate genes;
(C) dividing the copy number variation data from all training samples for all candidate segments into k subgroups, where k is a preselected number of folds;
(D) for each candidate segment, determining a mean classification error for the candidate segment, the determining including:
(i) for each of the k folds:
(1) designating a new one of the k-subgroups as an excluded testing subgroup and designate the remaining k-1 subgroups as training subgroups;
(2) training a naïve Bayesian (NB) classifier for the fold using the copy number variation data for the candidate segment in the k-1 training subgroups and testing the trained NB classifier using the copy number variation data for the one testing subgroup; and
(3) determining a classification error for the fold based on the results of testing;
(ii) determining the mean classification error for the candidate segment across the folds based on the classification error for each fold;
(E) selecting a current most relevant subset of the candidate segments based on the mean classification error for each candidate segment with the lowest mean classification error corresponding to the most relevant candidate segment;
(F) dividing the copy number variation data from all training samples for the selected most current relevant subset of the candidate segments subset of top scoring candidate segments into m subgroups, where m is a preselected number of folds, or using the same k folds and k subgroups as above for subsequent steps regarding m subgroups and m folds;
(G) training a geometric mean naïve Bayesian (GMNB) classifier based on the current most relevant subset of the candidate segments, and determining a mean measure of effectiveness based on an Area under the ROC curve (AUC) for the trained GMNB classifier across the m folds for the current most relevant subset of the candidate segments, including:
(i) for each of the m folds:
(1) designating a new one of the m-subgroups as an excluded testing subgroup and designating the remaining m-1 subgroups as training subgroups;
(2) training a GMNB classifier for the fold using the copy number variation data for the candidate segment in the m-1 training subgroups; and
(3) testing the trained GMNB classifier for the fold using the copy number variation data for the excluded testing subgroup resulting in a measure of effectiveness of the trained GMNB classifier for the fold; and
(ii) determining a mean measure of effectiveness of the trained GMNB classifier across the folds for the current most relevant subset of the candidate segments, which is referred to as the current measure of effectiveness for the current most relevant subset of the candidate segments;
(H) removing one or more of the least relevant candidate segments from the current most relevant subset of the candidate segments changing it into an immediately prior most relevant subset of candidate segments and forming a new current most relevant subset of the candidate segments, and labeling the current measure of effectiveness as the immediately prior measure of effectiveness for the immediately prior most relevant set of candidate segments; and
(I) repeating (G) for the new current most relevant subset of the candidate segments to determine a current measure of effectiveness for the current most relevant subset of the candidate segments;
where the current measure of effectiveness for the current most relevant subset of the candidate segments is statistically worse than the immediately prior measure of effectiveness for the immediately prior most relevant set of candidate segments, select the immediately prior most relevant set of candidate segments as the plurality of target segments;
where the current measure of effectiveness for the current most relevant subset of the candidate segments is better than or statistically the same as the immediately prior measure of effectiveness for the immediately prior most relevant set of candidate segments, performing (H) and (I) until the current measure of effectiveness for the current most relevant subset of the candidate segments is worse than the immediately prior measure of effectiveness for the immediately prior most relevant set of candidate segments, and
further comprising training a GMNB classifier on the plurality of target segments for some of the training data, for all of the training data, or for new training data to produce the trained classifier.

10. (canceled)

11. The method of claim 1, wherein the plurality of target genes are selected from Table 2; or
wherein at least some of the plurality of genes are selected from Table 2.

12. (canceled)

13. The method of claim 1, wherein the sample
(1) is a tumor sample or a solid tumor sample; and/or
(2) is a tissue biopsy of the cancer or a liquid biopsy; and/or
(3) comprises one or more of a tissue sample, a body fluid, or cell-free DNA or a tissue sample including surgical resection tissue or biopsy tissue from a tumor; and/or
(4) comprises a body fluid, and wherein the body fluid includes one or more of amniotic fluid, aqueous humor, bile, blood, blood plasma, a component of blood, cerebrospinal fluid, cerumen earwax cower's fluid pre-ejaculatory fluid, chyle, chyme stool, female ejaculate, interstitial fluid, intracellular fluid, lymph, menses, breast milk, mucus pleural fluid, peritoneal fluid, pus, saliva, sebum, semen, semen, sweat, synovial fluid, tears, urine, vaginal lubrication, vitreous humor, or vomit; and/or
(5) comprises bone marrow cells or peripheral blood cells.

14. (canceled)

15. (canceled)

16. (canceled)

17. (canceled)

18. (canceled)

19. (canceled)

20. (canceled)

21. (canceled)

22. (canceled)

23. The method of claim 1, wherein identifying the subject as a candidate for treatment with a double strand break-inducing agent comprises one or more of:
displaying on a graphical user interface an identification of the subject as a candidate for treatment with a double strand break-inducing agent;
storing data identifying the subject as a candidate for treatment with a double strand break-inducing agent;
sending an electronic communication including an identification of the subject as a candidate for treatment with a double strand break-inducing agent;
displaying on a graphical user interface a recommendation of treatment with a double strand break-inducing agent chemotherapy or immunotherapy for the subject;
storing data including a recommendation of treatment with a double strand break-inducing agent chemotherapy or immunotherapy for the subject; or
sending an electronic communication including a recommendation of treatment with a double strand break-inducing agent chemotherapy or immunotherapy for the subject.

24. A non-transitory computer-readable medium storing instructions that, when executed by one or more processors of a computing system, perform steps including:
a) determining sequences of segments and copy number for the sequences of a plurality of target genes in a sample obtained from a subject who has cancer using next generation sequencing;
b) determining copy number variation for each of a plurality of target segments from the determined sequences and copy number for the sequences of the plurality of target genes; and
c) at least one of:
(1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or
(2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or
(3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 gene or the BRCA2 gene by applying the trained classifier and using the copy number variation for the plurality of target segments as input attributes for the trained classifier, and
wherein the cancer is a breast cancer; or an ovarian cancer; or one or more of lymphoma, leukemia, or a solid tumor.

25. The non-transitory computer-readable medium of claim 24, wherein the steps further comprise, at least one of:
(1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or
(2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; or
(3) where the sample is classified as having a mutation of the BRCA1 or the BRCA2 gene or a genomic structural abnormality biologically similar to a mutation of the BRCA1 or the BRCA2 gene indicating a similar level of HRD as that caused by a mutation of the BRCA1 or the BRCA2 gene irrespective of the mutated gene involved, identifying the subject as a candidate for treatment with a double strand break-inducing agent.

26. The non-transitory computer-readable medium of claim 24, wherein the steps further comprise, at least one of:
(1) where the sample is classified as not having a high level of HRD and therefore not having the same or similar structural abnormality as a level of HRD associated with a mutation of the BRCA1 or the BRCA2 gene irrespective of the mutated gene involved, identifying the subject as not a candidate for treatment with a double strand break-inducing agent; or
(2) where the sample is classified as not having a mutation of the BRCA1 or the BRCA2 gene or a level of HRD similar to that caused by a mutation of the BRCA1 or the BRCA2 gene irrespective of the mutated gene involved, identifying the subject as not a candidate for treatment with a double strand break-inducing agent; or
(3) where the sample is classified as not having a mutation of the BRCA1 or the BRCA2 gene or a genomic structural abnormality biologically similar to a mutation of the BRCA1 gene or the BRCA2 gene indicating a similar level of HRD as that caused by a mutation of the BRCA1 gene or the BRCA2 gene irrespective of the mutated gene involved, identifying the subject as not a candidate for treatment with a double strand break-inducing agent, and/or
wherein the training data for the trained classifier further comprises a third group of samples with mutations in one double strand break repair gene.

27. The non-transitory computer-readable medium of claim 24, wherein the training data for the trained classifier comprises a first group of samples with confirmed mutations in the BRCA1 and/or the BRCA2 genes, and a second group of samples that are confirmed negative for mutations in the BRCA1 and the BRCA2 genes and confirmed negative for mutations in any double strand break repair genes.

28. (canceled)

29. The non-transitory computer-readable medium of claim 24, wherein the trained classifier is a geometric mean naïve Bayesian classifier, or wherein the steps further comprise training a classifier to produce the trained classifier.

30. (canceled)

31. The non-transitory computer-readable medium of any one of claim 24, wherein the steps further comprise determining the plurality of target segments whose copy number variation is used for classification by steps including:
(A) accessing or obtaining training samples including a first group of samples with confirmed mutations in the BRCA1 and/or the BRCA2 genes, and a second group of samples that are confirmed negative for mutations in the BRCA1 and the BRCA2 genes and confirmed negative for mutations in any double strand break repair genes;
(B) for each training sample,
(i) determining sequences and copy number of a plurality of candidate genes in the training sample using next generation sequencing; and
(ii) determining copy number variation for each of a plurality of candidate segments from the determined sequences and copy number of the plurality of candidate genes;
(C) dividing the copy number variation data from all training samples for all candidate segments into k subgroups, where k is a preselected number of folds;
(D) for each candidate segment, determining a mean classification error for the candidate segment, the determining including:
(i) for each of the k folds:
(1) designating a new one of the k-subgroups as an excluded testing subgroup and designate the remaining k-1 subgroups as training subgroups;
(2) training a naïve Bayesian (NB) classifier for the fold using the copy number variation data for the candidate segment in the k-1 training subgroups and testing the trained NB classifier using the copy number variation data for the one testing subgroup; and
(3) determining a classification error for the fold based on the results of testing;
(ii) determining the mean classification error for the candidate segment across the folds based on the classification error for each fold;
(E) selecting a current most relevant subset of the candidate segments based on the mean classification error for each candidate segment with the lowest mean classification error corresponding to the most relevant candidate segment;
(F) dividing the copy number variation data from all training samples for the selected most current relevant subset of the candidate segments subset of top scoring candidate segments into m subgroups, where m is a preselected number of folds, or using the same k folds and k subgroups as above for subsequent steps regarding m subgroups and m folds;
(G) training a geometric mean naïve Bayesian (GMNB) classifier based on the current most relevant subset of the candidate segments, and determining a mean measure of effectiveness based on an Area under the ROC curve (AUC) for the trained GMNB classifier across the m folds for the current most relevant subset of the candidate segments, including:
(i) for each of the m folds:
(1) designating a new one of the m-subgroups as an excluded testing subgroup and designating the remaining m-1 subgroups as training subgroups;
(2) training a GMNB classifier for the fold using the copy number variation data for the candidate segment in the m-1 training subgroups; and
(3) testing the trained GMNB classifier for the fold using the copy number variation data for the excluded testing subgroup resulting in a measure of effectiveness of the trained GMNB classifier for the fold; and
(ii) determining a mean measure of effectiveness of the trained GMNB classifier across the folds for the current most relevant subset of the candidate segments, which is referred to as the current measure of effectiveness for the current most relevant subset of the candidate segments;
(H) removing one or more of the least relevant candidate segments from the current most relevant subset of the candidate segments changing it into an immediately prior most relevant subset of candidate segments and forming a new current most relevant subset of the candidate segments, and labeling the current measure of effectiveness as the immediately prior measure of effectiveness for the immediately prior most relevant set of candidate segments; and
(I) repeating (G) for the new current most relevant subset of the candidate segments to determine a current measure of effectiveness for the current most relevant subset of the candidate segments;
where the current measure of effectiveness for the current most relevant subset of the candidate segments is statistically worse than the immediately prior measure of effectiveness for the immediately prior most relevant set of candidate segments, select the immediately prior most relevant set of candidate segments as the plurality of target segments;
where the current measure of effectiveness for the current most relevant subset of the candidate segments is statistically better than or statistically the same as the immediately prior measure of effectiveness for the immediately prior most relevant set of candidate segments, performing (H) and (I) until the current measure of effectiveness for the current most relevant subset of the candidate segments is worse than the immediately prior measure of effectiveness for the immediately prior most relevant set of candidate segments, and wherein the steps further comprise training a GMNB classifier on the plurality of target segments for some of the training data, for all of the training data, or for new training data to produce the trained classifier.

32. (canceled)

33. The non-transitory computer-readable medium of claim 24, wherein the plurality of target genes are selected from Table 2; or
wherein at least some of the plurality of genes are selected from Table 2.

34. (canceled)

35. The non-transitory computer-readable medium of claim 24, wherein the sample
(1) is a tumor sample or a solid tumor sample; and/or
(2) is a tissue biopsy of the cancer or a liquid biopsy; and/or
(3) comprises one or more of a tissue sample, a body fluid, or cell-free DNA or a tissue sample including surgical resection tissue or biopsy tissue from a tumor; and/or
(4) comprises a body fluid, and wherein the body fluid includes one or more of amniotic fluid, aqueous humor, bile blood, blood plasma, a component of blood, cerebrospinal fluid, cerumen, earwax, cowper's fluid, pre-Ejaculatory fluid, chyle, chyme, stool, female ejaculate interstitial fluid intracellular fluid, lymph, menses, breast milk, mucus, pleural fluid, peritoneal fluid, pus, saliva, sebum semen, serum sweat, synovial fluid, tears, urine, vaginal lubrication vitreous humor or vomit; and/or
(5) corn rises bone marrow cells or peripheral blood cells.

36. (canceled)

37. (canceled)

38. (canceled)

39. (canceled)

40. (canceled)

41. (canceled)

42. (canceled)

43. (canceled)

44. (canceled)

45. The non-transitory computer-readable medium of claim 24, wherein identifying the subject as a candidate for treatment with a double strand break-inducing agent comprises one or more of:
displaying on a graphical user interface an identification of the subject as a candidate for treatment with a double strand break-inducing agent;
storing data identifying the subject as a candidate for treatment with a double strand break-inducing agent;
sending an electronic communication including an identification of the subject as a candidate for treatment with a double strand break-inducing agent;
displaying on a graphical user interface a recommendation of treatment with a double strand break-inducing agent chemotherapy or immunotherapy for the subject;
storing data including a recommendation of treatment with a double strand break-inducing agent chemotherapy or immunotherapy for the subject; and
sending an electronic communication including a recommendation of treatment with a double strand break-inducing agent chemotherapy or immunotherapy for the subject.

46. A system comprising:
storage;
one or more processors in communication with the storage and configured to execute instructions from the storage, that, when executed, provide one or more modules including:
a sequencing and copy number variation (CNV) module configured to determine sequences and copy number for the sequences for a plurality of target genes in a sample from a subject who has a cancer using next generation sequencing; and
a classification module configured to:
obtain CNV data for a plurality of target sequences from the sequencing and CNV module; and
at least one of:
(1) classifying the sample as having a high level of homologous recombination deficiency (HRD) and therefore having a same biological abnormality as a level of HRD associated with a mutation of a BRCA1 or a BRCA2 gene irrespective of the mutated gene involved, or as not having a high level of HRD and therefore not having the same biological abnormality as the level of HRD associated with a mutation of the BRCA1 or the BRCA2 gene, irrespective of the mutated gene involved, by applying a trained classifier and using the copy number variation for the plurality of target segments as input attributes for the trained classifier; or
(2) classifying the sample as having a mutation of the BRCA1 or the BRCA2 gene or a level of homologous recombination deficiency (HRD) similar to that caused by a mutation of the BRCA1 or the BRCA2 gene, irrespective of the mutated gene involved, or as not having a mutation of the BRCA1 gene or the BRCA2 gene or a level of HRD similar to that caused by a mutation of the BRCA1 or the BRCA2 gene, irrespective of the mutated gene involved, by applying the trained classifier and using the copy number variation for the plurality of target segments as input attributes for the trained classifier; or
(3) classifying the sample as having a mutation of the BRCA1 or the BRCA2 gene or genomic structural abnormalities similar to a mutation of the BRCA1 or the BRCA2 gene, irrespective of the mutated gene involved, indicating a similar level of homologous recombination deficiency (HRD) as that caused by a mutation of the BRCA1 or the BRCA2 gene, or as not having a mutation of the BRCA1 or the BRCA2 gene or genomic structural abnormalities similar to a mutation of the BRCA1 or the BRCA2 gene by applying the trained classifier and using the copy number variation for the plurality of target segments as input attributes for the trained classifier, and
wherein the cancer is a breast cancer; or an ovarian cancer; or one or more of lymphoma, leukemia, or a solid tumor.

47. The system of claim 46, wherein the classification module is further configured to perform one or more of:
(1) where the sample is classified as having a high level of HRD and therefore the same or similar structural abnormality as a level of HRD associated with a mutation of the BRCA1 gene or the BRCA2 gene irrespective of the mutated gene involved, identifying the subject as a candidate for treatment with a double strand break-inducing agent; or
(2) where the sample is classified as having a mutation of the BRCA1 gene or the BRCA2 gene or a level of HRD similar to that caused by a mutation of the BRCA1 or the BRCA2 gene irrespective of the mutated gene involved, identifying the subject as a candidate for treatment with a double strand break-inducing agent; or
(3) where the sample is classified as having a mutation of the BRCA1 or the BRCA2 gene or a genomic structural abnormality biologically similar to a mutation of the BRCA1 or the BRCA2 gene indicating a similar level of HRD as that caused by a mutation of the BRCA1 or the BRCA2 gene irrespective of the mutated gene involved, identifying the subject as a candidate for treatment with a double strand break-inducing agent.

48. The system of claim 46, wherein the classification module is further configured to perform at least one of:
(1) where the sample is classified as not having a high level of HRD and therefore not having the same or similar structural abnormality as a level of HRD associated with a mutation of the BRCA1 or the BRCA2 gene irrespective of the mutated gene involved, identifying the subject as not a candidate for treatment with a double strand break-inducing agent; or
(2) where the sample is classified as not having a mutation of the BRCA1 or the BRCA2 gene or a level of HRD similar to that caused by a mutation of the BRCA1 or the BRCA2 gene irrespective of the mutated gene involved, identifying the subject as not a candidate for treatment with a double strand break-inducing agent; or
(3) where the sample is classified as not having a mutation of the BRCA1 or the BRCA2 gene or a genomic structural abnormality biologically similar to a mutation of the BRCA1 gene or the BRCA2 gene indicating a similar level of HRD as that caused by a mutation of the BRCA1 gene or the BRCA2 gene irrespective of the mutated gene involved, identifying the subject as not a candidate for treatment with a double strand break-inducing agent.

49. The system of claim 46, wherein
the training data for the trained classifier comprises a first group of samples with confirmed mutations in the BRCA1 and/or the BRCA2 gene, and a second group of samples that are confirmed negative for mutations in the BRCA1 and the BRCA2 gene and confirmed negative for mutations in any double strand break repair genes, and/or
wherein the training data for the trained classifier further comprises a third group of samples with mutations in one double strand break repair gene.

50. (canceled)

51. The system of any one of claim 46, wherein the trained classifier is a geometric mean naïve Bayesian classifier.

52. The system of any one of claim 46, further comprising a classifier training module configured to produce the trained classifier, and/or wherein producing the trained classifier comprises determining the plurality of target segments whose copy number variation is used for classification by steps including:
(A) accessing or obtaining training samples including a first group of samples with confirmed mutations in the BRCA1 and/or the BRCA2 gene, and a second group of samples that are confirmed negative for mutations in the BRCA1 and the BRCA2 gene and confirmed negative for mutations in any double strand break repair genes:
(B) for each training sample,
(i) determining sequences and copy number of a plurality of candidate genes in the training sample using next generation sequencing; and
(ii) determining copy number variation for each of a plurality of candidate segments from the determined sequences and copy number of the plurality of candidate genes:
(C) dividing the copy number variation data from all training samples for all candidate segments into k subgroups, where k is a preselected number of folds;
(D) for each candidate segment, determining a mean classification error for the candidate segment, the determining including:
(i) for each of the k folds:
(1) designating a new one of the k-subgroups as an excluded testing subgroup and designate the remaining k-1 subgroups as training subgroups;
(2) training a naïve Bayesian (NB) classifier for the fold using the copy number variation data for the candidate segment in the k-1 training subgroups and testing the trained NB classifier using the copy number variation data for the one testing subgroup; and
(3) determining a classification error for the fold based on the results of testing:
(ii) determining the mean classification error for the candidate segment across the folds based on the classification error for each fold:
(E) selecting a current most relevant subset of the candidate segments based on the mean classification error for each candidate segment with the lowest mean classification error corresponding to the most relevant candidate segment:
(F) dividing the copy number variation data from all training samples for the selected most current relevant subset of the candidate segments subset of top scoring candidate segments into m subgroups, where m is a preselected number of folds, or using the same k folds and k subgroups as above for subsequent steps regarding m subgroups and m folds:
(G) training a geometric mean naïve Bayesian (GMNB) classifier based on the current most relevant subset of the candidate segments, and determining a mean measure of effectiveness based on an Area under the ROC curve (AUC) for the trained GMNB classifier across the m folds for the current most relevant subset of the candidate segments, including:
(i) for each of the m folds:
(1) designating a new one of the m-subgroups as an excluded testing subgroup and designating the remaining m-1 subgroups as training subgroups:
(2) training a GMNB classifier for the fold using the copy number variation data for the candidate segment in the m-1 training subgroups; and
(3) testing the trained GMNB classifier for the fold using the copy number variation data for the excluded testing subgroup resulting in a measure of effectiveness of the trained GMNB classifier for the fold; and
(ii) determining a mean measure of effectiveness of the trained GMNB classifier across the folds for the current most relevant subset of the candidate segments, which is referred to as the current measure of effectiveness for the current most relevant subset of the candidate segments:
(H) removing one or more of the least relevant candidate segments from the current most relevant subset of the candidate segments changing it into an immediately prior most relevant subset of candidate segments and forming a new current most relevant subset of the candidate segments, and labeling the current measure of effectiveness as the immediately prior measure of effectiveness for the immediately prior most relevant set of candidate segments; and
(I) repeating (G) for the new current most relevant subset of the candidate segments to determine a current measure of effectiveness for the current most relevant subset of the candidate segments:
where the current measure of effectiveness for the current most relevant subset of the candidate segments is statistically worse than the immediately prior measure of effectiveness for the immediately prior most relevant set of candidate segments, select the immediately prior most relevant set of candidate segments as the plurality of target segments:
where the current measure of effectiveness for the current most relevant subset of the candidate segments is statistically better than or statistically the same as the immediately prior measure of effectiveness for the immediately prior most relevant set of candidate segments, performing (H) and (I) until the current measure of effectiveness for the current most relevant subset of the candidate segments is worse than the immediately prior measure of effectiveness for the immediately prior most relevant set of candidate segments.

53. (canceled)

54. The system of any one of claim 46, wherein the plurality of target genes are selected from Table 2; or
wherein at least some of the plurality of genes are selected from Table 2.

55. (canceled)

56. The system of claim 46, wherein the sample
(1)_is a tumor sample or a solid tumor sample; and/or
(2) is a tissue biopsy of the cancer or a liquid biopsy; and/or
(3) comprises one or more of a tissue sample, a body fluid, or cell-free DNA or a tissue sample including surgical resection tissue or biopsy tissue from a tumor; and/or
(4) comprises a body fluid, and wherein the body fluid includes one or more of amniotic fluid, aqueous humor, bile, blood, blood plasma, a component of blood, cerebrospinal fluid cerumen earwax cower's fluid re-ejaculatory fluid, chyle, chyme, stool, female ejaculate, interstitial fluid, intracellular fluid, lymph, menses, breast milk, mucus pleural fluid, peritoneal fluid, pus saliva, sebum, semen, serum sweat, synovial fluid, tears, urine, vaginal lubrication, vitreous humor, or vomit; and/or
(5) comprises bone marrow cells or peripheral blood cells.

57. (canceled)

58. (canceled)

59. (canceled)

60. (canceled)

61. (canceled)

62. (canceled)

63. (canceled)

64. (canceled)

65. (canceled)

66. The system of claim 46, further comprising one or more of:
a graphical user interface configured to display an identification of the subject as a candidate for treatment with a double strand break-inducing agent, to display a recommendation of treatment with a double strand break-inducing agent chemotherapy or immunotherapy for the subject, or both;
storage configured to store data identifying the subject as a candidate for treatment with a double strand break-inducing agent, to store data including a recommendation of treatment with a double strand break-inducing agent chemotherapy or immunotherapy for the subject, or both; or
a communication module configured to send an electronic communication including an identification of the subject as a candidate for treatment with a double strand break-inducing agent, configured to send an electronic communication including a recommendation of treatment with a double strand break-inducing agent chemotherapy or immunotherapy for the subject, or both.
